(19) United States
(12) Patent Application Publication
(10) Pub. No.: US 2025/0135035 A1
(43) Pub. Date: May 1, 2025

(54) MRNA REGULON THERAPY FOR THE TREATMENT OF HAPLOINSUFFICIENCY DISORDERS

(71) Applicant: The Johns Hopkins University, Baltimore, MD (US)

(72) Inventor: Jeffery Coller, Frederick, MD (US)

(21) Appl. No.: 18/693,636

(22) PCT Filed: Sep. 20, 2022
(86) PCT No.: PCT/US2022/076723

§ 371 Date: Mar. 20, 2024

Related U.S. Application Data
(60) Provisional application No. 63/352,530, filed on Jun. 15, 2022.
(60) Provisional application No. 63/246,689, filed on Sep. 21, 2021.

(51) Int. Cl. A61K 48/00; A61K 38/17; A61K 38/46; C07K 14/47; C12N 9/22

(52) U.S. Cl. CPC A61K 48/0058; A61K 38/1709; A61K 38/465; C07K 14/47; C12N 9/22

(57) ABSTRACT

Described herein are compositions and methods for treatment of haploinsufficiency disorders by mRNA regulation. For example, provided herein are fusion proteins that include an RNA effector protein that targets mRNA(s) of an active allele of a gene associated with a haploinsufficiency disorder; and a regulon moiety that stimulates and/or stabilizes the mRNA(s).

### CROSS-REFERENCE TO RELATED APPLICATIONS

This application claims priority to U.S. Provisional Patent Application No. 63/246,689, filed on Sep. 21, 2021, and U.S. Provisional Patent Application No. 63/352,530, filed on Jun. 15, 2022, which are incorporated herein by reference in their entireties.

### SEQUENCE LISTING

This application contains a Sequence Listing that has been submitted electronically as an XML file named “44807-0408WO1.XML.” The XML file, created on Sep. 20, 2022, is 468,769 bytes in size. The material in the XML file is hereby incorporated by reference in its entirety.

### TECHNICAL FIELD

Described herein are compositions and methods for treatment of haploinsufficiency disorders by mRNA regulation.

### BACKGROUND

Haploinsufficiency occurs when one gene allele is inactivated and the amount of gene product expressed from the remaining active allele is insufficient for proper gene function. A number of disorders are associated with, or are caused by haploinsufficiency. An example of a haploinsufficiency disorder is Dravet Syndrome.

Dravet Syndrome is a rare and catastrophic form of intractable epilepsy that begins in infancy. Initially, patients experience prolonged seizures. In their second year, additional types of seizure begin to occur, which typically coincide with a developmental decline, possibly due to repeated cerebral hypoxia. This leads to poor development of language and motor skills. Mutations in SCNIA (encoding the voltage-gated sodium channel a subunit), SCNIB (encoding the voltage-gated sodium channel ˜I subunit), SCN2A, SCN3A, SCN9A, GABRG2 (encoding the y-aminobutyric acid receptor y2 subunit), GABRD (encoding the y-aminobutyric acid receptor 11 subunit) and/or PCDH19 genes have been linked to Dravet Syndrome.

SCN1A encodes the neuronal voltage-gated sodium channel Nav1.1 that is expressed prominently in inhibitory GABAergic neurons. Loss-of-function (LOF) mutations in SCN1A including missense and premature termination codons (PTC) are the most frequently discovered cause of Dravet Syndrome [1, 2].

LOF premature termination codons (PTCs) arise from single nucleotide mutations that convert a canonical triplet nucleotide codon into one of three stop codons (TAG, TGA, or TAA). PTCs can be more deleterious than missense mutations because they result in the complete loss of protein expression [7]. There are ten amino acid codons vulnerable to PTC conversion by a single nucleotide substitution (arginine, tryptophan, tyrosine, cysteine, glutamic acid, lysine, glutamine, serine, leucine, and glycine). PTC mutations in SCN1A are a prevalent class of mutations associated with Dravet Syndrome (FIG. 1) with more than 180 known PTC mutations.

SCN1A LOF mutations lead to neuronal network hyperactivity as a consequence of impaired inhibitory GABAergic neurotransmission. While frequent unprovoked seizures are among the presenting feature of Dravet Syndrome, patients also suffer from a range of comorbidities affecting the areas of cognition, locomotion, speech, and behavior [3]. Dravet Syndrome patients often have disrupted sleep and circadian rhythms, neurodevelopmental delay and intellectual disabilities, oculomotor deficits, and psychomotor regression. Sudden unexpected death associated with epilepsy (SUDEP) is also prevalent in this population [4-6]. Due to the severity of these comorbidities, effective treatments that can address both seizures and the range of comorbidities associated with Dravet Syndrome are urgently needed. Dravet Syndrome has available animal models and has been amenable to clinical trials with successful outcomes.

There are limited therapeutic options for repair of mutations in SCN1A. Small molecules, such as aminoglycosides [9], dipeptides [10], and oxadiazoles promote “read-through” but this approach results in the encoding of a near-cognate amino acid [12], effectively generating a missense mutation in place of the PTC. Sodium channels, such as SCN1A, are poorly tolerant of missense mutations, which can cause gain-of-function and loss-of-function neurological disorders [13, 14]. Furthermore, aminoglycosides are ototoxic and nephrotoxic [15], and the first-in-class oxadiazole (Ataluren) displayed unexpectedly low efficacy in patient populations (ACT DMD Phase 3 clinical trial, NCT01826487; ACT CF, NCT02139306). Furthermore, most previous therapeutic initiatives for Dravet Syndrome were aimed only at seizure reduction. True disease modifying therapies for developmental and epileptic encephalopathies (DEE) are lacking.

Recent and ongoing advances in CRISPR/Cas9-mediated genome editing provides a potentially permanent solution for monogenic diseases. However, LOF missense mutations occur throughout the SCN1A coding region, making gene-editing approaches highly challenging [8]. Furthermore, aspects of this technology impart hurdles for its rapid use as a therapeutic [16, 17] including cell type specific delivery, the efficiency of homologous recombination, and the frequency of on-target repair errors and off-target editing. Moreover, at >6 kb per transcript, the sodium channel coding region of SCN1A exceeds the capacity of AAV thus limiting the utility of the rapid pace of advancing AAV therapies for tissue specific gene replacement. Thus, there are limited clinical options for SCN1A LOF associated with Dravet Syndrome, and the therapeutic pipeline remains thin. The compositions and methods described herein address this and other deficiencies in the art.

### SUMMARY

The compositions and methods described herein have a broad range of applications, providing potential disease modifying therapies for a range of haploinsufficiency disorders. The key advantage of this mRNA regulon approach is that it is 100% agnostic to mutation type and location by stabilizing the mRNA of the wild-type (WT) allele. The potential to reengineer these approaches for other indications is high and facile.

Thus, provided herein are fusion proteins comprising: an RNA effector protein that targets mRNA(s) of an active allele of a gene associated with a haploinsufficiency disorder; and a regulon moiety that stimulates and/or stabilizes the mRNA(s).

In some embodiments, the RNA effector protein is a Cas effector protein selected from the group consisting of Cas9, Cas12, Cas13, and Cas14. In some embodiments, the RNA effector protein is Cas13b. In some embodiments, the Cas effector protein is a catalytically inactive Cas protein.

In some embodiments, the regulon moiety is PABPC1 or NAT10.

In some embodiments, the fusion proteins further comprise a linker and/or a spacer.

In some embodiments, the fusion proteins further comprise a nuclear export signal and/or an epitope tag.

In some embodiments, the RNA effector protein is N terminal to the regulon moiety. In some embodiments, the RNA effector protein is C terminal to the regulon moiety.

In some embodiments, the fusion proteins comprise or consist of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

Also provided herein are polynucleotides encoding any one of the fusion proteins described herein. Also provided herein are vectors comprising any one of the polynucleotides described herein. Also provided herein are cells comprising any one of the vectors described herein.

Also provided herein are systems comprising: a fusion protein comprising: an RNA effector protein that targets mRNA(s) of an active allele of a gene associated with a haploinsufficiency disorder; and a regulon moiety that stimulates and/or stabilizes the mRNA(s); and a gRNA that forms a complex with the RNA effector protein and comprises a complementarity region that hybridizes with the mRNA(s) of the active allele.

In some embodiments, the RNA effector protein is a Cas effector protein selected from the group consisting of Cas9, Cas12, Cas13, and Cas14. In some embodiments, the Cas effector protein is Cas13b. In some embodiments, the Cas effector protein is a catalytically inactive Cas effector protein.

In some embodiments, the regulon moiety is PABPC1 or NAT10.

In some embodiments, the fusion protein further comprises a linker and/or a spacer. In some embodiments, the fusion protein further comprises a nuclear export signal and/or an epitope tag.

In some embodiments, the RNA effector protein is N terminal to the regulon moiety. In some embodiments, the RNA effector protein is C terminal to the regulon moiety.

In some embodiments, the fusion protein comprises or consists of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

In some embodiments, the gRNA targets an mRNA encoding MeCP2, SCN1A, SYNGAP1, SHANK3, CHD2, or PTEN. In some embodiments, the gRNA comprises a complementarity region designed to hybridize to an mRNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof. In some embodiments, the gRNA comprises a complementarity region designed to hybridize to an mRNA selected from the group consisting of SEQ ID NO: 4, SEQ ID NO: 6, SEQ ID NO: 10, SEQ ID NO: 15, SEQ ID NO: 17, and combinations thereof.

In some embodiments, the gRNA comprises a complementarity region designed to hybridize to an mRNA encoding an amino acid selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

In some embodiments, the gRNA comprises a complementarity region designed to hybridize to an mRNA selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

In some embodiments, the gRNA comprises a complementarity region designed to hybridize to an mRNA encoding an amino acid selected from the group consisting of SEQ ID NO: 37, SEQ ID NO: 38, SEQ ID NO: 39, SEQ ID NO: 40, SEQ ID NO: 41, and combinations thereof.

In some embodiments, the gRNA is selected from the group consisting of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and SEQ ID NO: 72.

Also provided herein are one or more polynucleotide(s) encoding any one of the systems described herein. Also provided herein are one or more vector(s) comprising any one of the polynucleotide(s) described herein. Also provided herein are cells comprising any one of the vector(s) described herein.

Also provided herein are complexes comprising: a fusion protein comprising: an RNA effector protein that targets mRNA(s) of an active allele of a gene associated with a haploinsufficiency disorder; and a regulon moiety that stimulates and/or stabilizes the mRNA(s), bound to a gRNA comprising a complementarity region that hybridizes with the mRNA(s) of the active allele.

In some embodiments, the RNA effector protein is dCas13b and the regulon moiety is PABP1 or NAT10.

In some embodiments, the gRNA comprises a complementarity region designed to hybridize to an mRNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof. In some embodiments, the gRNA comprises a complementarity region designed to hybridize to an mRNA selected from the group consisting of SEQ ID NO: 4, SEQ ID NO: 6, SEQ ID NO: 10, SEQ ID NO: 15, SEQ ID NO: 17, and combinations thereof.

In some embodiments, the gRNA comprises a complementarity region designed to hybridize to an mRNA encoding an amino acid selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

In some embodiments, the gRNA comprises a complementarity region designed to hybridize to an mRNA selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

In some embodiments, the gRNA comprises a complementarity region designed to hybridize to an mRNA encoding SEQ ID NO: 37, SEQ ID NO: 38, SEQ ID NO: 39, SEQ ID NO: 40, SEQ ID NO: 41, and combinations thereof.

Also provided herein are complexes comprising: a fusion protein comprising: an RNA effector protein that targets mRNA(s) of an active allele of a gene associated with a haploinsufficiency disorder; and a regulon moiety that stimulates and/or stabilizes the mRNA(s), bound to a gRNA and the mRNA.

In some embodiments, the RNA effector protein is dCas13b and the regulon moiety is PABP1 or NAT10.

In some embodiments, the mRNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof. In some embodiments, the mRNA encodes an amino acid selected from the group consisting of SEQ ID NO: 4, SEQ ID NO: 6, SEQ ID NO: 10, SEQ ID NO: 15, SEQ ID NO: 17, and combinations thereof.

In some embodiments, the mRNA is selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

In some embodiments, the mRNA is selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

In some embodiments, the mRNA encodes an amino acid selected from the group consisting of is selected from the group consisting of SEQ ID NO: 37, SEQ ID NO: 38, SEQ ID NO: 39, SEQ ID NO: 40, SEQ ID NO: 41, and combinations thereof.

Also provided herein are pharmaceutical compositions comprising: any one of the fusion proteins or systems described herein. In some embodiments, the pharmaceutical compositions further comprise a pharmaceutically acceptable carrier.

Also provided herein are pharmaceutical compositions comprising: one or more nucleic acids encoding any one of the fusion proteins or systems described herein.

Also provided herein are viral vectors comprising one or more nucleic acids encoding any one of the fusion proteins or systems described herein. In some embodiments, the viral vector is an adeno-associated viral vector.

Also provided herein are nanoparticles or liposomes comprising any one of the fusion proteins or systems described herein or one or more nucleic acids encoding any one of the fusion proteins or systems described herein.

Also provided herein are methods of stimulating or stabilizing mRNA(s), the method comprising: contacting the mRNA(s) with any one of the fusion proteins or systems described herein. In some embodiments, the method is carried out, in vitro, in vivo, or ex vivo.

Also provided herein are methods of treating or preventing a haploinsufficiency disorder in a subject, the method comprising: administering to the subject a fusion protein or a nucleic acid encoding a fusion protein comprising: an RNA effector protein that targets mRNA(s) of an active allele of a gene associated with a haploinsufficiency disorder; and a regulon moiety that stimulates and/or stabilizes the mRNA(s); and a gRNA or a nucleic acid encoding a gRNA designed to form a complex with the RNA effector protein and comprising a complementarity region designed to hybridize with the mRNA of the active allele.

In some embodiments, the RNA effector protein is a Cas effector protein selected from the group consisting of a Cas9, Cas12, Cas13, and Cas14. In some embodiments, the Cas effector protein is Cas13b. In some embodiments, the Cas effector protein is a catalytically inactive Cas protein.

In some embodiments, the regulon moiety is PABPC1 or NAT10.

In some embodiments, the fusion protein further comprises a linker and/or a spacer. In some embodiments, the fusion protein further comprises a nuclear export signal and/or an epitope tag.

In some embodiments, the RNA effector protein is N terminal to the regulon moiety. In some embodiments, the RNA effector protein is C terminal to the regulon moiety.

In some embodiments, the fusion protein comprises or consists of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

In some embodiments, the haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A,Brain malformations with or without urinary tract defects (BRMUTD), Carney complex type 1,CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, adrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrome type 4C.

In some embodiments, the haploinsufficiency disorder is a CNS haploinsufficiency disorder. In some embodiments, the CNS haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof. In some embodiments, the haploinsufficiency disorder is Dravet Syndrome or Rett syndrome.

In some embodiments, the subject has a haploinsufficiency in a gene selected from the group consisting of AGGFI, ARHGAP31, BMPR2, CHD7, COL2Al, COL3Al, CTLA4, CTNNBI, DLL4, EHMTI, ELN, ENG, FAS, FBNI, FOXGI, GATA3, GLI3, GRN, IRF6, JAGI, KCNQ4, LMXIB, MBD5, MED13L, MITF, MNXI, MYCN, NFIA, NFIX, NOTCH!, NSDI, PAX3, PHIP, PRKARIA, RAil, RBPJ, RPS14, RUNX2, SALL4, SCN1A, SETBPI, SHANK3, SHH, SHOX, SLC2AI/GLUT1, SOXI0, SYNGAPI, TBXI, TBX3, TBX5, TCF4, TCOFI, TGIFI, TNXB, TRPSI, WTI, ZIC2, and combinations thereof. In some embodiments, the subject has a haploinsufficiency in a gene selected from the group consisting of SCN1A, SCN2A, SCN8A, SCN12A5, SPTAN1, CDKL5, CHD2, FOXG1, KCNQ2, PCDH19, SLC6A1, STXBP1, SYNGAP1, CACNA1A, DEPDC5, MECP2, TSC1, TSC2, and combinations thereof.

In some embodiments, the subject has mutation selected from the list in Table 4 and combinations thereof. In some embodiments, the subject has a mutation selected from the list in Table 6 and combinations thereof. In some embodiments, the subject is a mammal. In some embodiments, the subject is a human.

In some embodiments, the fusion protein and gRNA are administered as part of a pharmaceutical composition. In some embodiments, administering comprises administering a viral vector comprising nucleic acid sequence(s) encoding the fusion protein and gRNA to the subject. In some embodiments, administering comprises administering a nanoparticle or liposome comprising the fusion protein and gRNA or nucleic acid sequence(s) encoding the fusion protein and gRNA to the subject.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic showing SCN1A topology and premature-termination codons (PTC). Each of the 182 SCN1A PTCs is show by color.

FIG. 2 is a schematic representing the tethered function assay. The fused functional protein to the tethered protein bind to the mRNA of interest to enhance or stabilize the mRNA.

FIG. 3A is a schematic showing the experimental approach applied for this assay. Briefly, PABPC1 was fused to dCas13b tethering protein and guided to 3′UTR of the Luciferase gene via designed guide RNAs (gRNA) to enhance its translation.

FIG. 3B shows a firefly luciferase assessment depicting about 2-folds increase in PAB-tethered luciferase targeting over non-targeting in HEK-293 cells.

FIG. 4 shows a map of a plasmid pJC1211 encoding a Cas13b-PABPC1 fusion protein.

FIGS. 5A-5AB show the sequence and features of the plasmid pJC1211 (SEQ ID NO: 50). The nucleic acid sequence is shown in both 5′→3′ and the reverse complement (3′→5′).

FIG. 6 is a map of plasmid pJC1212 encoding a luciferase targeting Cas13b gRNA.

FIGS. 7A-7G show the sequence and features of plasmid pJC1212 (SEQ ID NO: 52). The nucleic acid sequence is shown in both 5′→3′ and the reverse complement (3′→5′).

FIG. 8 is a map of plasmid pJC1213 encoding a luciferase targeting Cas13b gRNA.

FIGS. 9A-9F show the sequence and features of plasmid pJC1213 (SEQ ID NO: 53).

FIG. 10 is a map of Addgene plasmid 103854 (empty vector as compared to pJC1212 and pJC1213).

FIGS. 11A-11H show the sequence and features of Addgene plasmid 103854 (empty vector as compared to pJC1212 and pJC1213) (SEQ ID NO: 54).

FIGS. 12A-12G shows the fusion protein approach enhances mRNA expression in multiple cell types. FIG. 12A is an exemplary schematic representing the tethered mRNA amplifier approach. Briefly, PABPC1 is fused to dCas13b. This fusion is recruited to specific mRNAs via a guide RNA (gRNA) targeted to the 3′UTR. In HEK293 cells, the Tethered mRNA Amplifier stimulates gRNA-dependent luciferase reporter (FIG. 12B) and endogenous MeCP2 mRNA (FIG. 12C) expression, using either a luciferase activity assay or western blot, respectively. Red bars in the corresponding schematics represent the approximate positions of two gRNAs used in each experiment. The effect of the Tethered mRNA Amplifier requires PABPC1 (FIG. 12D). The Tethered mRNA Amplifier also enhances MeCP2 mRNA levels (FIG. 12E). A similar stimulatory effect on MeCP2 can also be seen in SH-SY5Y and HepG2 cells (FIG. 12F). FIG. 12G shows that the position of the gRNA along the MeCP2 3′UTR alters the stimulatory effect. (p-values: *<0.05, **<0.005, ***<0.0005).

FIGS. 13A-13D show the fusion protein enhances the expression of haploinsufficiency disorder-associated transcripts; SYNGAP1 (FIG. 13A), SHANK3 (FIG. 13B), PTEN (FIG. 13C), and CHD2 (FIG. 13D) mRNAs were targeted by the Tethered mRNA Amplifier in a gRNA-dependent manner. The result of protein and mRNA analysis in SH-SY5Y cells are shown. All the protein assays were performed with four distinct biological replicates and at least two biological repeats for the RNA analysis. (p-values: *<0.05, **<0.005, ***<0.0005).

FIGS. 14A-14F show a minimal fusion protein functions in cells. FIG. 14A is an exemplary schematic of PABPC1 and its functional motifs. RRM1-4 are RNA Recognition Motifs. MLLE is the Mademoiselle domain. FIG. 14B shows AlphaFold predicted model of dCas13b fusion with the MLLE domain of PABPC1 (amino acids 545-636). FIG. 14C shows a predicted model of full-length PABPC1-all residues except amino acids 545-636 have been hidden post-prediction. FIG. 14D shows a predicted model of full-length dCas13b alone. FIG. 14E shows PyMol alignment of FIGS. 14B-14D. FIG. 14F shows Western blots comparing tethering of full-length PABPC1 and MLLE domain alone targeting MeCP2 transcripts in HEK293 cells.

FIG. 15 is an exemplary schematic showing haploinsufficiency disorders result when one copy of a gene is mutated while the other is normal. This mutation ultimately reduces protein expression by one half, causing a disease state. The mRNA Amplifier targets the messenger RNA from the normal gene theoretically doubling protein expression to normal amounts.

### DETAILED DESCRIPTION

The compositions and methods described herein have a broad range of applications, providing potential disease modifying therapies for a range of haploinsufficiency disorders. Haploinsufficiency occurs when one gene allele is inactivated and the amount of gene product expressed from the remaining active allele is insufficient for proper gene function. A number of disorders are associated with, or are caused by haploinsufficiency. An example of a haploinsufficiency disorder is Dravet Syndrome.

In the treatment of haploinsufficiencies, targeting messenger RNA metabolism offers a novel therapeutic window. Though changes in gene expression are commonly considered to reflect programmed transcriptional variability. Less commonly considered is that extensive regulation of messenger RNA expression also occurs during translation. For example, in the early oocyte, large changes in protein expression occur via coordinated activation/deactivation of maternally derived mRNA. Likewise, translationally quiescent transcripts are activated upon synaptic stimulation in neurons. A strong discordance between mRNA levels and protein levels is also observed in somatic cells, highlighting the prevalence and physiological significance of a post-transcriptional regulon.

Transcript stability and translation rate are a key feature defining the post-transcriptional regulon. All transcripts degrade and are translated at unique rates. mRNA stability and translation are often proportional to gene function; housekeeping genes typically produce long lived mRNAs that persist in cells for over 24 hours, whereas transiently expressed genes generate short-lived transcripts that survive on the order of minutes. In part, the cell achieves this vast repertoire of transcript stabilities and expression though sequence and/or structural elements that recruit specific positive or negative acting factors. The compositions and methods described herein, among other things, utilize this mRNA/post transcriptional regulon as a novel disease modifying treatment for haploinsufficiency disorders such as Dravet Syndrome.

mRNA Regulation Therapeutics

Described herein are novel molecular therapeutic strategies using posttranscriptional regulation of mRNA, e.g., SCN1A mRNA, to overcome haploinsufficiency in genetic brain disorders. The theory of the technology is based on what is termed “A Tethered Function Approach” or a “Tethered mRNA Amplifier Approach”. In brief, this technology tethers a specific factor to the 3′ untranslated region (3′UTR) of a target mRNA, thereby changing its stability and/or translational rate. The approach is commonly used to determine the function of RNA binding proteins [18, 19].

Proteins and protein complexes that regulate mRNA metabolism possess two activities. They bind an mRNA specifically, and then elicit some function, that is, regulate mRNA splicing, transport, localization, translation, or stability. These two activities can often reside in different proteins in a complex, or in different regions of a single polypeptide. In the majority of instances involving mRNAs, specific RNA binding activity and function are distinct. In these cases, the separation of the two activities from one another provides a powerful inroad for a therapeutic modality. In essence, a functional activity (stripped of its native RNA binding activity) can be tethered to a new mRNA via a unique RNA binding activity.

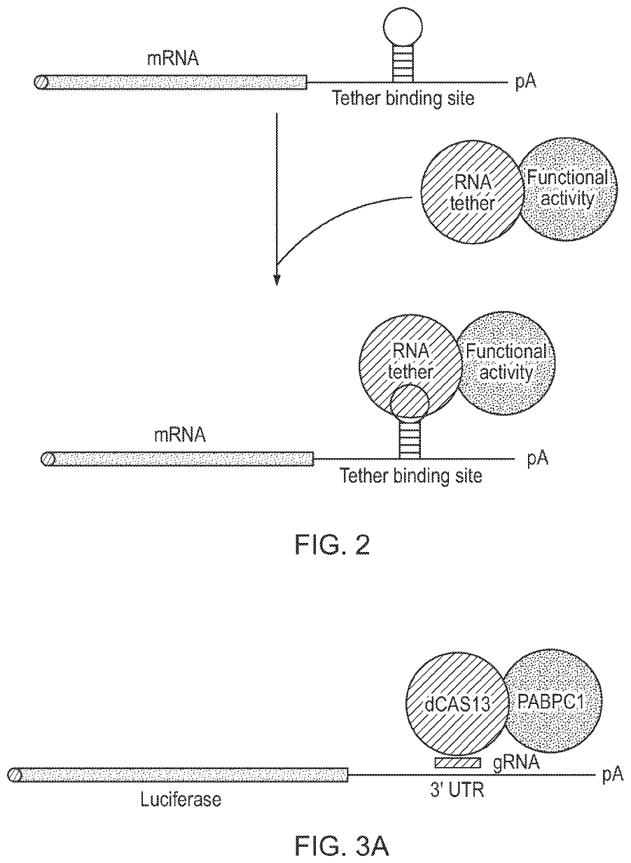

In the tethered function assay (e.g., as shown in FIG. 2), to determine the effects of a protein X on mRNA metabolism, a chimeric protein is expressed in vivo in which protein X is continuous with a tethering polypeptide (see FIG. 2). The tethering protein is an RNA-binding protein that recognizes an RNA tag sequence with high specificity and affinity. The effect of the fusion protein on mRNA metabolism is determined by co-expressing the chimera with an mRNA reporter (such as lacZ or luciferase) into which a tag RNA sequence has been embedded. The fusion protein's effects on mRNA metabolism are assayed by conventional means [e.g., Western blot, Northern blot, reverse transcriptase polymerase chain reaction (RT-PCR), etc.]. The assay, however, until now, has only been utilized in model organisms/cell culture to evaluate the contribution of RNA binding proteins to the post-transcriptional regulation of mRNA. Described herein, is a novel therapeutic modality, based on tethering, for disease modification, e.g. for modification of Dravet Syndrome.

Tethered function assays were developed as a tool to dissect the function of unknown RNA binding protein in the posttranscriptional control of mRNA. The contrived system took advantage of exogenous RNA binding activities, often of bacteriophage origin (MS2 Coat Protein and Lamda N-peptide being the most common). The obvious limitation to this approach was that a corresponding RNA binding element had to be cloned and expressed in a reporter mRNA. The novel Protein/RNA binding was utilized to “tether” an activity to an mRNA and test its function. Critically, having the ability to tether a functional activity to ANY mRNA of interest would be powerful in changing that mRNAs expression pattern. And this approach could be leveraged in the treatment of haploinsufficiency.

CRISPR-based research tools established a system capable of selective recognition of RNA molecules. This novel RNA targeting system has enabled the detecting and manipulation of specific RNA using different approaches, such as RNA-knock-down, site-specific RNA editing, RNA localization, and destruction of toxic RNAs that lead to human neurodegenerative disorders [20, 21].

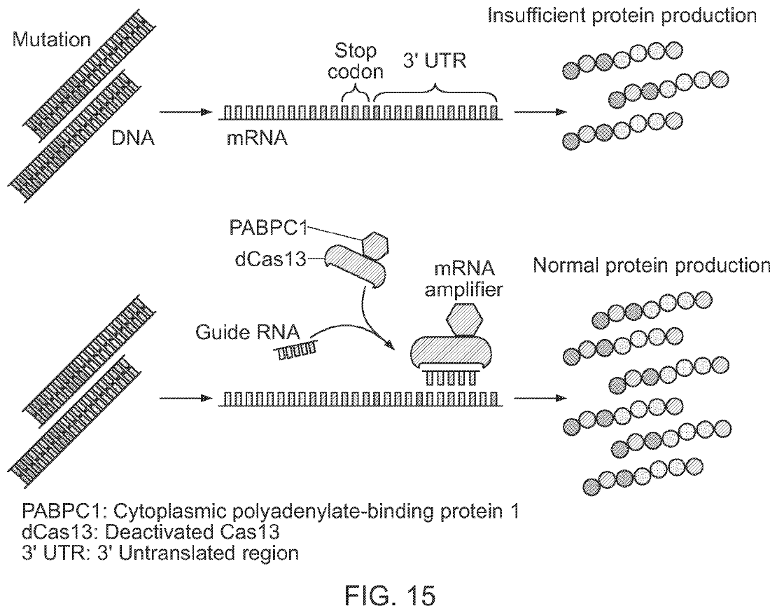

In some cases, the mRNA regulon therapy described herein utilize the CRISPR-based RNA binding approach, providing a precise and advanced technology to target genetic disorders at the transcript level. The RNA targeting CRISPR-Cas13 has RNA strand specificity and binds with high affinity; enabling efficient and precise delivery to relevant utilize the mRNA-specific targeting capacity of Cas13b to tether known factors that enhance mRNA expression to ameliorate haploinsufficiency (FIG. 15).

### Haploinsufficiency Disorders and Genes

In some cases, the haploinsufficiency disorder is selected from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A,Brain malformations with or without urinary tract defects (BRMUTD), Carney complex type 1,CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, adrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrometype 4C. In some cases, the haploinsufficient disorder is Dravet syndrome.

In some cases, the haploinsufficient gene is selected from AGGFI, ARHGAP 31, BMPR2, CHD7, COL2Al, COL3Al, CTLA4, CTNNBI, DLL4, EHMTI, ELN, ENG, FAS, FBNI, FOXG1, GATA3, GLI3, GRN, IRF6, JAGI, KCNQ4, LMXIB, MBD5, MED13L, MITF, MNXI, MYCN, NFIA, NFIX, NOTCH!, NSDI, PAX3, PHIP, PRKARIA, RAil, RBPJ, RPS14, RUNX2, SALL4, SCN1A, SETBPI, SHANK3, SHH, SHOX, SLC2Al/GLUT1, SOXI0, SYNGAPI, TBXI, TBX3, TBX5, TCF4, TCOFI, TGIFI, TNXB, TRPSI, WTI, ZIC2, and combinations thereof. In some cases embodiments, the haploinsufficient gene is SCN1A.

In some cases, the combination of haploinsufficiency disorder and haploinsufficient gene combination is a combination shown in Table 1.

TABLE 1
Haploinsufficiency disorders and genes.

| | Haploinsufficient |
|---|---|
| Haploinsufficiency Disorder | Gene |
| 5q-syndrome | RPS14 |
| Adams-Oliver syndrome I | ARHGAP31 |
| Adams-Oliver syndrome 3 | RBPJ |
| Adams-Oliver syndrome 5 | NOTCH1 |
| Adams-Oliver syndrome 6 | DLL4 |
| Alagille syndrome I | JAG1 |
| Autoimmune lymphoproliferative syndrome type IA | FAS |
| Autoimmune lymphoproliferative syndrome type V | CTLA4 |
| Autosomal dominant deafness-2A | KCNQ4 |
| Brain malformations with or without urinary tract defects (BRMUTD) | NFIA |
| Carney complex type 1 | PRKARlA |
| CHARGE syndrome | CHD7 |
| Cleidocranial dysplasia | RUNX2 |
| Currarino syndrome | MNXl |
| Denys-Drash syndrome/Frasier syndrome | WTl |
| Developmental delay, intellectual | PHIP |
| disability, obesity, and dysmorphic features (DIDOD) | |
| DiGeorge syndrome (TBXl-associated) | TBXl |
| Dravet syndrome | SCNlA |
| Duane-radial ray syndrome | SALL4 |
| Ehlers-Danlos syndrome (classic-like) | TNXB |
| Ehlers Danlos syndrome (vascular type) | COL3Al |
| Feingold syndrome 1 | MYCN |
| Frontotemporal lobar degeneration with TDP43 inclusions (FTLD- | GRN |
| TDP), GRN-related | |
| GLUT1 deficiency syndrome | SLC2Al |
| Greig cephalopolysyndacty1y syndrome | GLI3 |
| Hereditary hemorrhagic telangiectasia type 1 | ENG |
| Holoprosencephaly 3 | SHH |
| Holoprosencephaly 4 | TGIFl |
| Holoprosencephaly 5 | ZIC2 |
| Holt-Oram syndrome | TBX5 |
| Hypoparathyroidism, sensorineural deafness, and renal disease (HDR) | GATA3 |
| Kleefstra syndrome 1 | EHMTl |
| Klippel-Trenaunay syndrome (AAGF-related) | AGGFl |
| Leri-Weill dyschondrosteosis | SHOX |
| Marfan syndrome | FBNl |
| Mental retardation and distinctive facial features with or without | MED13L |
| cardiac defects (MRFACD) | |
| Mental retardation, autosomal dominant 1 | MBD5 |
| Mental retardation, autosomal dominant 19 | CTNNBl |
| Mental retardation, autosomal dominant 29 | SETBPl |
| Nail-patella syndrome (NPS) | LMXlB |
| Phelan-McDermid syndrome | SHANK3 |
| Pitt-Hopkins syndrome | TCF4 |
| Primary pulmonary hypertension 1 | BMPR2 |
| Rett syndrome (congenital variant) | FOXGl |
| Smith-Magenis syndrome (RAIl-associated) | RAI1 |
| Sotos syndrome 1 | NSDl |
| Sotos syndrome 2 | NFIX |
| Stickler syndrome type I | COL2Al |
| Supravalvular aortic stenosis | ELN |
| SYNGAPl-related intellectual disability | SYNGAPl |
| Treacher Collins syndrome | TCOFl |
| Trichorhinophalangeal syndrome type I | TRPSl |
| Ulnar-mammary syndrome | TBX3 |
| van der Woude syndrome 1 | IRF6 |
| Waardenburg syndrome type 1 | PAX3 |
| Waardenburg syndrome type 2A | MITF |
| Waardenburg syndrome type 4C | SOXlO |

### CNS Haploinsufficiency Disorders

In some cases, the haploinsufficiency disorder is a CNS haploinsufficiency disorder. In some cases, the haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof.

In some cases, the haploinsufficiency gene is selected from the group consisting of SCN1A, SCN2A, SCN8A, SCN12A5, SPTAN1, CDKL5, CHD2, FOXG1, KCNQ2, PCDH19, SLC6A1, STXBP1, SYNGAP1, CACNA1A, DEPDC5, MECP2, TSC1, TSC2, and combinations thereof.

In some cases, the combination of haploinsufficiency disorder and haploinsufficient gene combination is a combination shown in Table 2

TABLE 2
CNS haploinsufficiency disorders and genes

| Haploinsufficiency Disorder | Haploinsufficient Gene |
|---|---|
| Episodic ataxia, Familial hemiplegia migraine | CACNA1A |
| CDKL5 deficiency disorder | CDKL5 |
| CHD2 myoclonic encephalopathy | CHD2 |
| Lennox-Gastaut Syndrome | CHD2 |
| Familial focal epilepsy with variable loci | DEPDC5 |
| FOXG1 syndrome | FOXG1 |
| Benign familial neonatal seizures | KCNQ2 |
| Rett syndrome | MECP2 |
| Dravet syndrome | SCN1A |
| SCN2A-epileptic encephalopathy | SCN2A |
| SCN2A-developmental encephalopathy | SCN2A |
| SCN8A-epileptic encephalopathy | SCN8A |
| SCN8A familial infantile epilepsy | SCN8A |
| Early infantile epileptic encephalopathy | SLC12A5 |
| Myoclonic-atonic epiplepsy | SLC6A1 |
| Early infantile epileptic encephalopathy | SPTAN1 |
| SYNGAP1-related intellectual disability | SYNGAP1 |
| Tuberous sclerosis | TSC1 |
| Tuberous sclerosis | TSC2 |
| KCNQ2-related epileptic encephalopathy | KCNQ2 |
| SLC6A1-related myoclonic-astatic epilepsy | SLC6A1 |
| STXBP1-related epileptic encephalopathy | STXBP1 |
| SYNGAP1 syndrome | SYNGAP1 |

### SCN1A

SCN1A (NCBI Gene ID: 6323; RefSeq NG_011906.1) encodes a sodium voltage-gated channel alpha subunit 1. Its transcripts and protein isoforms shown in Table 3.

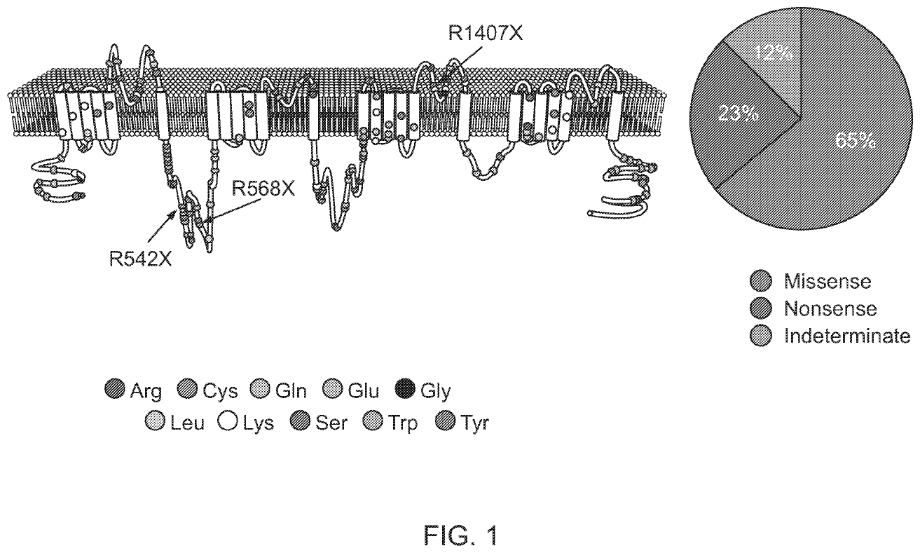

FIG. 1 is a schematic showing SCN1A topology and premature-termination codons (PTC).

TABLE 3
SCN1A transcripts and isoforms

| NCBI transcript | Tran- | SEQ ID | NCBI protein | Isoform | SEQ ID |
|---|---|---|---|---|---|
| accession | script # | NO: | accession | # | NO: |
| NR_148667.2 | 16 | SEQ ID | | | |
| | | NO: 1 | | | |
| XR_001738884.1 | X2 | SEQ ID | | | |
| | | NO: 2 | | | |
| XR_001738883.1 | X1 | SEQ ID | | | |
| | | NO: 3 | | | |
| NM_001353949.2 | 6 | SEQ ID | NP_ | 2 | SEQ ID |
| | | NO: 4 | 001340878.1 | | NO: 21 |
| NM_001353958.2 | 13 | SEQ ID | NP_ | 3 | SEQ ID |
| | | NO: 5 | 001340887.1 | | NO: 20 |
| NM_001353950.2 | 7 | SEQ ID | NP_ | 2 | SEQ ID |
| | | NO: 6 | 001340879.1 | | NO: 21 |
| NM_001202435.3 | 4 | SEQ ID | NP_ | 1 | SEQ ID |
| | | NO: 7 | 001189364.1 | | NO: 19 |
| NM_001353955.2 | 11 | SEQ ID | NP_ | 4 | SEQ ID |
| | | NO: 8 | 001340884.1 | | NO: 22 |
| NM_001353957.2 | 12 | SEQ ID | NP_ | 3 | SEQ ID |
| | | NO: 9 | 001340886.1 | | NO: 20 |
| NM_001353951.2 | 8 | SEQ ID | NP_ | 2 | SEQ ID |
| | | NO: 10 | 001340880.1 | | NO: 21 |
| NM_001353948.2 | 5 | SEQ ID | NP_ | 1 | SEQ ID |
| | | NO: 11 | 001340877.1 | | NO: 19 |
| NM_001353960.2 | 14 | SEQ ID | NP_ | 5 | SEQ ID |
| | | NO: 12 | 001340889.1 | | NO: 23 |
| NM_001353954.2 | 10 | SEQ ID | NP_ | 4 | SEQ ID |
| | | NO: 13 | 001340883.1 | | NO: 22 |
| NM_001165964.3 | 3 | SEQ ID | NP_ | 3 | SEQ ID |
| | | NO: 14 | 001159436.1 | | NO: 20 |
| NM_001353952.2 | 9 | SEQ ID | NP_ | 2 | SEQ ID |
| | | NO: 15 | 001340881.1 | | NO: 21 |
| NM_001353961.2 | 15 | SEQ ID | NP_ | 6 | SEQ ID |
| | | NO: 16 | 001340890.1 | | NO: 24 |
| NM_006920.6 | 2 | SEQ ID | NP_ | 2 | SEQ ID |
| | | NO: 17 | 008851.3 | | NO: 21 |
| NM_001165963.4 | 1 | SEQ ID | NP_ | 1 | SEQ ID |
| | | NO: 18 | 001159435.1 | | NO: 19 |

Mutations in SCN1A that are associated with Dravat Syndrome are shown in Table 4.

TABLE 4
Mutations in SCN1A associated with Dravat Syndrome

| Position(s) in | |
|---|---|
| SEQ ID NO: 19 | Description |
| 17 | Missing in DRVT. |
| 58 | G → V in DRVT. |
| 61 | L → F in DRVT. |
| 63 | F → L in DRVT. Corresponds to variant dbSNP:rs121917907 |
| 68 | I → T in DRVT; borderline phenotype. Corresponds to |
| | variant dbSNP:rs758871507 |
| 78 | E → D in DRVT. Corresponds to variant dbSNP:rs121917933 |
| 79 | D → H in DRVT; borderline phenotype. Corresponds to |
| | variant dbSNP:rs121917982 |
| 79 | D → N in DRVT. Corresponds to variant dbSNP:rs121917982 |
| 84 | Y → C in DRVT. 5 Publications Corresponds to |
| | variant dbSNP:rs121917964 |
| 90 | F → S in DRVT and ICEGTC. Corresponds to |
| | variant dbSNP:rs121918733 |
| 91 | I → T in DRVT. Corresponds to variant dbSNP:rs121918734 |
| 98 | A → P in DRVT. |
| 101 | R → Q in DRVT and ICEGTC. 8 Publications Corresponds to |
| | variant dbSNP:rs121917918 |
| 101 | R → W in DRVT. Corresponds to variant dbSNP:rs121917965. |
| 103 | S → G in DRVT. Corresponds to variant dbSNP:rs121918743 |
| 105 | T → I in DRVT. Corresponds to variant dbSNP:rs796053089 |
| 108 | L → R in DRVT. |
| 112 | T → I in DRVT. Corresponds to variant dbSNP:rs121918745 |
| 113 | P → T in DRVT. Corresponds to variant dbSNP:rs794726711 |
| 118 | R → S in DRVT. Corresponds to variant dbSNP:rs121917959 |
| 124 | I → N in DRVT. Corresponds to variant dbSNP:rs121918761 |
| 127 | H → D in DRVT; borderline phenotype. Corresponds to |
| | variant dbSNP:rs148442069 |
| 162 | T → P in DRVT. Corresponds to variant dbSNP:rs121917934 |
| 171 | I → K in DRVT. Corresponds to variant dbSNP:rs121918766 |
| 171 | I → R in DRVT. |
| 175 | A → T in DRVT. Corresponds to variant dbSNP:rs121918767 |
| 175 | A → V in DRVT. |
| 177 | G → E in DRVT; results in a non-functional channel. Corresponds to |
| | variant dbSNP:rs121918770 |
| 179 | C → R in DRVT. |
| 190 | W → R in DRVT. Corresponds to variant dbSNP:rs121918773 |
| 191 | N → K in DRVT. |
| 191 | N → Y in DRVT. Corresponds to variant dbSNP:rs121918762 |
| 194 | D → G in DRVT. |
| 194 | D → N in DRVT. Corresponds to variant dbSNP:rs121917935 |
| 199 | T → R in DRVT; borderline phenotype with spike wave |
| | activity. Corresponds to variant dbSNP:rs121917983 |
| 217 | T → K in DRVT. Corresponds to variant dbSNP:rs121917936 |
| 223 | A → E in DRVT. |
| 226 | T → M in DRVT; borderline phenotype; also found in a patient with |
| | cryptogenic generalized epilepsy. Corresponds to |
| | variant dbSNP:rs121917984 |
| 226 | T → R in DRVT. Corresponds to variant dbSNP:rs121917984 |
| 227 | I → S in DRVT; borderline phenotype with spike wave activity in some |
| | patients; results in a non-functional channel. Corresponds to |
| | variant dbSNP:rs121917937 |
| 227 | I → T in DRVT. |
| 228 | S → P in DRVT. Corresponds to variant dbSNP:rs1057519530 |
| 232 | G → S in DRVT. |
| 233 | L → R in DRVT. |
| 239 | A → T in DRVT; borderline phenotype with spike wave activity in |
| | some patients. Corresponds to variant dbSNP:rs121917985 |
| 239 | A → V in DRVT. Corresponds to variant dbSNP:rs121917909 |
| 243 | S → Y in DRVT. Corresponds to variant dbSNP:rs794726755 |
| 252 | I → N in DRVT. Corresponds to variant dbSNP:rs121918780 |
| 259 | S → R in DRVT. Corresponds to variant dbSNP:rs121918735 |
| 265 | G → W in DRVT. Corresponds to variant dbSNP:rs121918749 |
| 277 | C → R in DRVT. |
| 280 | W → C in DRVT. |
| 280 | W → R in DRVT. Corresponds to variant dbSNP:rs121917938 |
| 281 | P → A in DRVT. |
| 281 | P → L in DRVT. Corresponds to variant dbSNP:rs796052964 |
| 281 | P → S in DRVT. |
| 289 | E → V in DRVT. |
| 297 | T → I in DRVT. Corresponds to variant dbSNP:rs121918771 |
| 322 | R → I in DRVT. Corresponds to variant dbSNP:rs121917928 |
| 340 | S → F in DRVT. |
| 342 | A → V in DRVT. Corresponds to variant dbSNP:rs794726797 |
| 343 | G → D in DRVT. Corresponds to variant dbSNP:rs121918753 |
| 345 | C → R in DRVT. Corresponds to variant dbSNP:rs794726782 |
| 351 | C → W in DRVT. |
| 355 | G → D in DRVT. |
| 356 | R → G in DRVT. Corresponds to variant dbSNP:rs121917920 |
| 357 | N → I in DRVT. |
| 358 | P → T in DRVT. Corresponds to variant dbSNP:rs121917923 |
| 359 | N → S in DRVT and ICEGTC. Corresponds to |
| | variant dbSNP:rs794726713 |
| 363 | T → P in DRVT. Corresponds to variant dbSNP:rs1131691465 |
| 363 | T → R in DRVT. |
| 366 | D → E in DRVT. Corresponds to variant dbSNP:rs121917958 |
| 378 | L → Q in DRVT. |
| 379 | M → R in DRVT. |
| 383 | F → L in DRVT. Corresponds to variant dbSNP:rs121917939 |
| 384 | W → R in DRVT; borderline phenotype. Corresponds to |
| | variant dbSNP:rs1057523858 |
| 393 | R → C in DRVT; also in a patient with myoclonic astatic |
| | epilepsy. Corresponds to variant dbSNP:rs121917929 |
| 393 | R → H in DRVT and ICEGTC; results in a non-functional |
| | channel. Corresponds to variant dbSNP:rs121917927 |
| 393 | R → S in DRVT. Corresponds to variant dbSNP:rs121917929 |
| 400 | M → V in DRVT. |
| 400 | Missing in DRVT. |
| 403 | F → L in DRVT. Corresponds to variant dbSNP:rs121917966 |
| 403 | F → V in DRVT. |
| 406 | V → F in DRVT. Corresponds to variant dbSNP:rs121918768 |
| 409 | L → W in DRVT. |
| 413 | Y → N in DRVT. Corresponds to variant dbSNP:rs121917967 |
| 426 | Y → C in DRVT. Corresponds to variant dbSNP:rs796052973 |
| 426 | Y → N in DRVT; results in decreased peak current densities; causes a |
| | negative shift in the half-maximal steady-state inactivation and delayed |
| | recovery from fast inactivation. Corresponds to |
| | variant dbSNP:rs121917940 |
| 450-2009 | Missing in DRVT. |
| 525 | S → F in DRVT. |
| 626 | S → G in DRVT; also found in a patient with cryptogenic generalized |
| | epilepsy. Corresponds to variant dbSNP:rs121917990 |
| 674 | D → G in DRVT. |
| 762 | N → D in DRVT. |
| 783 | L → P in DRVT. Corresponds to variant dbSNP:rs121917968 |
| 785 | M → T in DRVT. Corresponds to variant dbSNP:rs796053095 |
| 812 | T → I in DRVT; borderline phenotype. |
| 812 | T → R in DRVT. Corresponds to variant dbSNP:rs121917941 |
| 842 | L → R in DRVT. |
| 843 | S → R in DRVT. |
| 846 | E → K in DRVT. Corresponds to variant dbSNP:rs121917942 |
| 854-855 | Missing in DRVT. |
| 859 | R → C in GEFS + 2 and DRVT; causes a positive shift in the voltage |
| | dependence of channel activation, slower recovery from slow inactivation |
| | and lower levels of current compared with the wild-type |
| | channel. Corresponds to variant dbSNP:rs121918784 |
| 862 | R → Q in DRVT. Corresponds to variant dbSNP:rs121918785 |
| 865 | R → G in DRVT; results in impaired channel fast inactivation and |
| | significantly increased persistent current. |
| 875 | T → K in DRVT. Corresponds to variant dbSNP:rs121918623 |
| 875 | T → M in GEFS + 2 and DRVT; borderline phenotype. Corresponds to |
| | variant dbSNP:rs121918623 |
| 876 | L → I in DRVT. |
| 890 | L → P in DRVT. Corresponds to variant dbSNP:rs1553541473 |
| 896 | V → F in DRVT; borderline phenotype. |
| 896 | V → L in DRVT. |
| 902 | F → C in DRVT. Corresponds to variant dbSNP:rs121918787 |
| 927 | C → F in DRVT. Corresponds to variant dbSNP:rs794726811 |
| 931 | R → C in DRVT. Corresponds to variant dbSNP:rs121918788 |
| 932 | W → C in DRVT. |
| 933 | H → P in DRVT. |
| 934 | M → I in DRVT. Corresponds to variant dbSNP:rs121918774 |
| 939 | H → P in DRVT; unknown pathological significance. |
| 939 | H → Q in DRVT; results in a non-functional channel. Corresponds to |
| | variant dbSNP:rs121918795 |
| 939 | H → Y in DRVT. Corresponds to variant dbSNP:rs121918736 |
| 940 | S → F in DRVT. Corresponds to variant dbSNP:rs1057521080 |
| 942 | L → P in DRVT. Corresponds to variant dbSNP:rs121917943 |
| 943 | I → N in DRVT. |
| 944 | V → A in DRVT and ICEGTC. Corresponds to |
| | variant dbSNP:rs121917969 |
| 944 | V → E in DRVT. |
| 945 | F → L in DRVT. Corresponds to variant dbSNP:rs121917970 |
| 946 | R → C in DRVT; loss-of-function mutation resulting in complete absence |
| | of sodium current. Corresponds to variant dbSNP:rs121918775 |
| 946 | R → H in DRVT and GEFS + 2; GEFS + 2 phenotype consists of partial |
| | epilepsy with antecedent febrile seizures and seizure aggravation by |
| | antiepileptic drugs; loss-of-function mutation resulting in complete absence |
| | of sodium current. Corresponds to variant dbSNP:rs121917971 |
| 946 | R → S in DRVT. Corresponds to variant dbSNP:rs121918775 |
| 949 | C → S in DRVT. |
| 949 | C → Y in DRVT. |
| 950 | G → E in DRVT. Corresponds to variant dbSNP:rs121917972 |
| 950 | G → R in DRVT. |
| 952 | W → G in DRVT. Corresponds to variant dbSNP:rs121918737 |
| 954 | E → K in DRVT. Corresponds to variant dbSNP:rs121918786 |
| 956 | M → K in DRVT. |
| 957 | W → L in DRVT. Corresponds to variant dbSNP:rs121917917 |
| 959 | C → R in DRVT; results in a non-functional channel. Corresponds to |
| | variant dbSNP:rs121918796 |
| 960 | M → V in DRVT. Corresponds to variant dbSNP:rs121918750 |
| 973 | M → K in DRVT. |
| 976 | M → I in DRVT and GEFS + 2. |
| 979 | G → V in DRVT. |
| 985 | N → I in DRVT. Corresponds to variant dbSNP:rs121918747 |
| 986 | L → F in DRVT; complete loss of function. Corresponds to |
| | variant dbSNP:rs121918625 |
| 986 | L → P in DRVT. |
| 987 | F → L in DRVT. |
| 993 | S → R in DRVT; borderline phenotype. |
| 998 | D → G in DRVT. Corresponds to variant dbSNP:rs1484321812 |
| 999-1000 | NL → LIS in DRVT. |
| 1068 | E → K in DRVT. |
| 1207 | L → P in DRVT. Corresponds to variant dbSNP:rs121917963 |
| 1208 | R → K in DRVT. Corresponds to variant dbSNP:rs1559149025 |
| 1210 | T → K in DRVT. Corresponds to variant dbSNP:rs121918738 |
| 1221 | E → K in DRVT. |
| 1230 | L → F in DRVT and GEFS + 2. |
| 1231 | S → R in DRVT. Corresponds to variant dbSNP:rs121918746 |
| 1231 | S → T in DRVT. Corresponds to variant dbSNP:rs121918800 |
| 1233 | G → R in DRVT. Corresponds to variant dbSNP:rs121917911 |
| 1238 | E → D in DRVT. Corresponds to variant dbSNP:rs121917973 |
| 1239 | D → G in DRVT. |
| 1239 | D → Y in DRVT. |
| 1245 | R → Q in DRVT. Corresponds to variant dbSNP:rs121917912 |
| 1255 | A → D in DRVT. |
| 1260 | T → P in DRVT. Corresponds to variant dbSNP:rs121918739 |
| 1263 | F → L in DRVT. Corresponds to variant dbSNP:rs121918752 |
| 1265 | L → P in DRVT. Corresponds to variant dbSNP:rs121918794 |
| 1266 | E → A in DRVT. |
| 1275 | G → V in DRVT. |
| 1284-2009 | Missing in DRVT. Add BLAST |
| 1284 | W → S in DRVT. |
| 1287 | L → P in DRVT. Corresponds to variant dbSNP:rs121918740 |
| 1288 | D → N in DRVT. |
| 1289 | Missing in DRVT; results in a non-functional channel. 3 Publications |
| 1316 | R → G in DRVT. |
| 1316 | R → S in DRVT. |
| 1320 | A → V in DRVT; borderline phenotype. |
| 1326 | A → P in DRVT. Corresponds to variant dbSNP:rs121918803 |
| 1328 | S → P in ICEGTC and DRVT. |
| 1335 | V → M in DRVT. Corresponds to variant dbSNP:rs121917960 |
| 1339 | A → V in DRVT. Corresponds to variant dbSNP:rs794726789 |
| 1344 | I → M in DRVT. |
| 1345 | P → L in DRVT. |
| 1350 | V → G in DRVT. |
| 1355 | L → P in DRVT. Corresponds to variant dbSNP:rs121918776 |
| 1358 | W → R in DRVT. |
| 1358 | W → S in DRVT. Corresponds to variant dbSNP:rs121917961 |
| 1367 | N → K in DRVT. Corresponds to variant dbSNP:rs121918760 |
| 1370 | A → P in DRVT; borderline phenotype. |
| 1378 | N → H in DRVT. |
| 1378 | N → T in DRVT. Corresponds to variant dbSNP:rs1131691775 |
| 1385 | F → V in DRVT. |
| 1390 | V → M in DRVT; some patients have a borderline DRVT |
| | phenotype. Corresponds to variant dbSNP:rs121917986 |
| 1391 | N → S in DRVT. Corresponds to variant dbSNP:rs1553525062 |
| 1393 | H → P in DRVT; borderline phenotype. |
| 1394 | T → I in DRVT. |
| 1396 | C → G in DRVT; some patients have a borderline DRVT |
| | phenotype. Corresponds to variant dbSNP:rs121917987 |
| 1396 | C → Y in DRVT. |
| 1414 | N → Y in DRVT. Corresponds to variant dbSNP:rs121917925 |
| 1416 | D → G in DRVT. |
| 1417 | N → S in DRVT. |
| 1418 | V → G in DRVT. |
| 1422 | Y → C in DRVT. Corresponds to variant dbSNP:rs121917913 |
| 1423 | L → F in DRVT. |
| 1426 | L → R in DRVT. Corresponds to variant dbSNP:rs121917944 |
| 1427 | Q → P in DRVT. |
| 1429 | Missing in DRVT. |
| 1431 | F → I in DRVT. |
| 1433 | G → E in DRVT. Corresponds to variant dbSNP:rs121918741 |
| 1433 | G → R in DRVT. Corresponds to variant dbSNP:rs121917908 |
| 1433 | G → V in DRVT. |
| 1434 | W → R in DRVT. Corresponds to variant dbSNP:rs121918789 |
| 1437 | I → M in DRVT. |
| 1441 | A → P in DRVT. Corresponds to variant dbSNP:rs121917974 |
| 1450 | Q → K in DRVT. Corresponds to variant dbSNP:rs121918806 |
| 1450 | Q → R in DRVT. Corresponds to variant dbSNP:rs121918790 |
| 1451 | P → L in DRVT. Corresponds to variant dbSNP:rs121917945 |
| 1451 | P → S in DRVT. |
| 1453 | Y → C in DRVT; borderline phenotype. |
| 1454 | E → K in DRVT. Corresponds to variant dbSNP:rs1553522472 |
| 1461 | L → I in DRVT. Corresponds to variant dbSNP:rs121918772 |
| 1462 | Y → C in DRVT. Corresponds to variant dbSNP:rs121917962 |
| 1462 | Y → H in DRVT and ICEGTC; borderline DRVT |
| | phenotype. Corresponds to variant dbSNP:rs1559119345 |
| 1463 | F → S in DRVT. Corresponds to variant dbSNP:rs121917946 |
| 1470 | G → W in DRVT. Corresponds to variant dbSNP:rs121917924 |
| 1472 | F → S in DRVT; borderline phenotype. |
| 1473 | Missing in DRVT. |
| 1475 | L → S in DRVT. Corresponds to variant dbSNP:rs121917947 |
| 1476 | N → K in DRVT; borderline phenotype. |
| 1483 | Missing in DRVT. |
| 1484 | D → G in DRVT. |
| 1485 | N → Y in DRVT. |
| 1503 | E → K in DRVT; borderline phenotype. 3 Publications |
| 1503 | Missing in DRVT; borderline phenotype. |
| 1514 | L → S in DRVT. Corresponds to variant dbSNP:rs121918764 |
| 1538 | V → I in DRVT. Corresponds to variant dbSNP:rs780360360 |
| 1544 | D → A in DRVT. |
| 1544 | D → G in DRVT. |
| 1545 | I → V in DRVT. Corresponds to variant dbSNP:rs121917975 |
| 1555 | M → R in DRVT. |
| 1559 | Missing in DRVT. |
| 1561 | E → K in DRVT. |
| 1579 | V → E in DRVT. |
| 1586 | G → E in DRVT. Corresponds to variant dbSNP:rs121918742 |
| 1588 | C → R in DRVT. Corresponds to variant dbSNP:rs121917919 |
| 1592 | L → H in DRVT; borderline phenotype. |
| 1592 | L → P in DRVT. |
| 1596 | R → C in DRVT; also found in a patient with cryptogenic focal |
| | epilepsy. Corresponds to variant dbSNP:rs121917993 |
| 1596 | R → L in DRVT. |
| 1605 | N → I in DRVT. Corresponds to variant dbSNP:rs1057519533 |
| 1605 | N → S in DRVT; borderline phenotype. |
| 1608 | D → G in DRVT. |
| 1608 | D → Y in DRVT. Corresponds to variant dbSNP:rs121917915 |
| 1612 | V → I in DRVT. Corresponds to variant dbSNP:rs121918808 |
| 1630 | V → L in DRVT; borderline phenotype in some patients. Corresponds to |
| | variant dbSNP:rs121917914 |
| 1630 | V → M in DRVT. Corresponds to variant dbSNP:rs121917914 |
| 1637 | V → E in DRVT; also found in a child with febrile status epilepticus who |
| | developed liver failure. Corresponds to variant dbSNP:rs121918810 |
| 1638 | I → N in DRVT. Corresponds to variant dbSNP:rs1057521079 |
| 1638 | I → T in DRVT; also found in a patient with an unclassified form of |
| | epilepsy. |
| 1639 | R → G in DRVT. |
| 1642 | R → S in DRVT. Corresponds to variant dbSNP:rs1131691581 |
| 1645 | R → Q in DRVT. Corresponds to variant dbSNP:rs121917976 |
| 1648 | R → C in DRVT. Corresponds to variant dbSNP:rs121918791 |
| 1648 | R → H in GEFS + 2 and DRVT. Corresponds to |
| | variant dbSNP:rs121918622 |
| 1653 | A → E in DRVT; borderline phenotype. |
| 1658 | T → M in DRVT. Corresponds to variant dbSNP:rs121917922 |
| 1658 | T → R in DRVT. Corresponds to variant dbSNP:rs121917922 |
| 1660 | L → P in DRVT. Corresponds to variant dbSNP:rs1131691675 |
| 1661 | F → S in DRVT. Corresponds to variant dbSNP:rs121918797 |
| 1662 | A → V in DRVT; borderline phenotype. Corresponds to |
| | variant dbSNP:rs794726839 |
| 1664 | M → K in DRVT. Corresponds to variant dbSNP:rs121918765 |
| 1667 | L → P in DRVT. Corresponds to variant dbSNP:rs1131691774 |
| 1668 | P → A in DRVT. Corresponds to variant dbSNP:rs121917948 |
| 1668 | P → L in DRVT; borderline phenotype. |
| 1672 | N → I in DRVT. |
| 1673 | I → T in DRVT. |
| 1674 | G → R in DRVT. Corresponds to variant dbSNP:rs121918792 |
| 1675 | L → R in DRVT. |
| 1677 | L → F in DRVT. |
| 1683 | I → T in DRVT; borderline phenotype. |
| 1684 | Y → D in DRVT. |
| 1685 | A → D in DRVT. Corresponds to variant dbSNP:rs121918744 |
| 1688 | G → W in DRVT. |
| 1692 | F → S in DRVT. Corresponds to variant dbSNP:rs121918778 |
| 1694 | Y → C in DRVT. Corresponds to variant dbSNP:rs121918777 |
| 1707 | F → V in DRVT. Corresponds to variant dbSNP:rs121917977 |
| 1713 | S → N in DRVT. Corresponds to variant dbSNP:rs121918816 |
| 1714 | M → K in DRVT. |
| 1714 | M → R in DRVT. Corresponds to variant dbSNP:rs121917949 |
| 1716 | C → R in DRVT. Corresponds to variant dbSNP:rs121917926 |
| 1721 | T → R in DRVT. Corresponds to variant dbSNP:rs121917978 |
| 1725 | G → C in DRVT. |
| 1726 | W → R in DRVT. Corresponds to variant dbSNP:rs121917979 |
| 1727 | D → G in DRVT. |
| 1741 | C → R in DRVT. |
| 1749 | G → E in DRVT. Corresponds to variant dbSNP:rs121918798 |
| 1756 | C → G in DRVT. Corresponds to variant dbSNP:rs121918809 |
| 1762 | G → E in DRVT. Corresponds to variant dbSNP:rs121917950 |
| 1763 | I → N in DRVT. |
| 1766 | Missing in DRVT. |
| 1770 | I → F in DRVT. |
| 1770 | I → N in DRVT. |
| 1770 | I → T in DRVT; borderline phenotype. |
| 1771 | I → F in DRVT; borderline phenotype; also found in a patient with focal |
| | epilepsy. |
| 1771 | I → N in DRVT. |
| 1773 | S → F in DRVT. Corresponds to variant dbSNP:rs121917951 |
| 1780 | M → T in DRVT. Corresponds to variant dbSNP:rs121917952 |
| 1781 | Y → C in DRVT and ICEGTC. Corresponds to |
| | variant dbSNP:rs121918779 |
| 1781 | Y → H in DRVT. |
| 1782 | I → M in DRVT. Corresponds to variant dbSNP:rs121918763 |
| 1782 | I → S in DRVT. |
| 1783 | A → T in DRVT. Corresponds to variant dbSNP:rs121917980 |
| 1783 | A → V in DRVT. Corresponds to variant dbSNP:rs121917921 |
| 1787 | E → K in DRVT. Corresponds to variant dbSNP:rs121917916 |
| 1788 | N → K in DRVT; unknown pathological significance. Corresponds to |
| | variant dbSNP:rs1381184010 |
| 1792 | A → T in DRVT. |
| 1807-1810 | Missing in DRVT. |
| 1808 | F → I in DRVT. |
| 1812-1815 | WEKF → C in DRVT. |
| 1812 | W → G in DRVT. Corresponds to variant dbSNP:rs121918751 |
| 1812 | W → S in DRVT. |
| 1813-1815 | Missing in DRVT. |
| 1831 | F → S in DRVT. Corresponds to variant dbSNP:rs121918748 |
| 1832 | A → P in DRVT. |
| 1835 | L → F in DRVT. |
| 1852 | M → K in DRVT. |
| 1855 | P → L in DRVT. Corresponds to variant dbSNP:rs1057517958 |
| 1880 | G → E in DRVT. Corresponds to variant dbSNP:rs201905405 |
| 1881 | E → D in DRVT. Corresponds to variant dbSNP:rs121918804 |
| 1909 | T → I in DRVT; functional channel displaying decreased peak current |
| | densities but increased persistent current. Corresponds to |
| | variant dbSNP:rs121918793 |
| 1909 | Missing in DRVT. Corresponds to variant dbSNP:rs1553519902 |
| 1922 | I → T in DRVT. Corresponds to variant dbSNP:rs121917981 |
| 1927 | R → IIQ in DRVT. |

### MECP2

MECP2 (NCBI Gene ID: 4204; RefSeq NG_007107.3) encodes a methyl-CpG binding protein 2. Its transcripts and protein isoforms are shown in Table 5.

TABLE 5
MECP2 transcripts and isoforms

| NCBI transcript | Tran- | SEQ ID | NCBI protein | Isoform | SEQ ID |
|---|---|---|---|---|---|
| accession | script # | NO: | accession | # | NO: |
| NM_001386139.1 | 10 | SEQ ID | NP_ | 4 | SEQ ID |
| | | NO: 25 | 001373068.1 | | NO: 39 |
| NM_001110792.2 | 2 | SEQ ID | NP_ | 2 | SEQ ID |
| | | NO: 26 | 001104262.1 | | NO: 37 |
| NM_001369393.2 | 6 | SEQ ID | NP_ | 3 | SEQ ID |
| | | NO: 27 | 001356322.1 | | NO: 38 |
| NM_001386138.1 | 9 | SEQ ID | NP_ | 4 | SEQ ID |
| | | NO: 28 | 001373067.1 | | NO: 39 |
| NM_004992.4 | 1 | SEQ ID | NP_ | 1 | SEQ ID |
| | | NO: 29 | 004983.1 | | NO: 40 |
| NM_001386137.1 | 8 | SEQ ID | NP_ | 4 | SEQ ID |
| | | NO: 30 | 001373066.1 | | NO: 39 |
| NM_001369392.2 | 5 | SEQ ID | NP_ | 3 | SEQ ID |
| | | NO: 31 | 001356321.1 | | NO: 38 |
| NM_001369391.2 | 4 | SEQ ID | NP_ | 3 | SEQ ID |
| | | NO: 32 | 001356320.1 | | NO: 38 |
| NM_001316337.2 | 3 | SEQ ID | NP_ | 3 | SEQ ID |
| | | NO: 33 | 001303266.1 | | NO: 38 |
| NM_001369394.2 | 7 | SEQ ID | NP_ | 3 | SEQ ID |
| | | NO: 34 | 001356323.1 | | NO: 38 |
| XM_024452383.1 | X1 | SEQ ID | XP_ | X1 | SEQ ID |
| | | NO: 35 | 024308151.1 | | NO: 41 |
| XM_011531166.2 | X3 | SEQ ID | XP_ | X1 | |
| | | NO: 36 | 011529468.1 | | |

Mutations in MECP2 associated with Rett disease are shown in Table 6.

TABLE 6
Mutations in MECP2 associated with Rett disease.

| Position(s) in | |
|---|---|
| SEQ ID NO: | |
| 40 | Description |
| 10 | E → Q in RTT. Corresponds to variant dbSNP:rs61754421. |
| 97 | D → E in RTT. Corresponds to variant dbSNP:rs61754449. |
| 97 | D → Y in RTT. Corresponds to variant dbSNP:rs61754448. |
| 100 | L → R in RTT. Corresponds to variant dbSNP:rs61754451. |
| 100 | L → V in RTT. Corresponds to variant dbSNP:rs28935168. |
| 101 | P → H in RTT. Corresponds to variant dbSNP:rs61754453. |
| 101 | P → L in RTT. Corresponds to variant dbSNP:rs61754453. |
| 101 | P → R in RTT; also in a patient with Angelman syndrome and some typical |
| | RTT features. Corresponds to variant dbSNP:rs61754453. |
| 101 | P → S in RTT. Corresponds to variant dbSNP:rs61754452. |
| 101 | P → T in RTT. |
| 106 | R → Q in RTT. Corresponds to variant dbSNP:rs61754457. |
| 106 | R → W in RTT. 1 Corresponds to variant dbSNP:rs28934907. |
| 111 | R → G in RTT. Corresponds to variant dbSNP:rs61754459. |
| 120 | Y → D in RTT. Corresponds to variant dbSNP:rs267608454. |
| 124 | L → F in RTT. Corresponds to variant dbSNP:rs61755763. |
| 128 | Q → P in RTT. Corresponds to variant dbSNP:rs61748383. |
| 133 | R → C in RTT; impairs interaction with ATRX and abolishes ATRX |
| | recruitment to heterochromatin. Corresponds to variant dbSNP:rs28934904. |
| 133 | R → H in RTT. Corresponds to variant dbSNP:rs61748389. |
| 134 | S → C in RTT. Corresponds to variant dbSNP:rs61748390. |
| 135 | K → E in RTT. Corresponds to variant dbSNP:rs61748391. |
| 152 | P → R in RTT. Corresponds to variant dbSNP:rs61748404. |
| 155 | F → I in RTT. Corresponds to variant dbSNP:rs61748406. |
| 155 | F → S in RTT. Corresponds to variant dbSNP:rs28934905. |
| 156 | D → G in RTT. Corresponds to variant dbSNP:rs61748407. |
| 158 | T → A in RTT. Corresponds to variant dbSNP:rs61748411. |
| 158 | T → M in RTT. Corresponds to variant dbSNP:rs28934906. |
| 161 | G → V in RTT. Corresponds to variant dbSNP:rs61748417. |
| 210 | K → I in RTT. Corresponds to variant dbSNP:rs61749730. |
| 225 | P → R in RTT. Corresponds to variant dbSNP:rs61749715. |
| 270-486 | Missing in RTT. Add BLAST |
| 302 | P → A in RTT. Corresponds to variant dbSNP:rs61751373. |
| 302 | P → H in RTT. Corresponds to variant dbSNP:rs61749723. |
| 302 | P → L in RTT. Corresponds to variant dbSNP:rs61749723. |
| 302 | P → R in RTT. Corresponds to variant dbSNP:rs61749723. |
| 305 | K → R in RTT; abolishes interaction with TBL1X. Corresponds to |
| | variant dbSNP:rs61751441. |
| 306 | R → C in RTT; abolishes interaction with TBL1X and |
| | TBL1XR1. Corresponds to variant dbSNP:rs28935468. |
| 306 | R → H in RTT. Corresponds to variant dbSNP:rs61751443. |
| 322 | P → A in RTT. Corresponds to variant dbSNP: rs61751449. |
| 322 | P → L in RTT. Corresponds to variant dbSNP:rs61751450. |
| 344 | R → W in RTT. Corresponds to variant dbSNP:rs61752361. |
| 388 | P → S in RTT; unknown pathological significance. Corresponds to |
| | variant dbSNP:rs61753000. |

### Fusion Proteins

Described herein are non-naturally occurring or engineered fusion proteins comprising an RNA effector protein and a regulation moiety, as well as variants and mutants thereof.

In some cases, the RNA effector protein amino acid sequence is N-terminal to the regulation moiety amino acid sequence. In some cases, the RNA effector protein amino acid sequence is C-terminal to the regulation protein amino acid sequence. In some cases, the RNA effector protein amino acid sequence is inserted within the regulation protein amino acid sequence. In some cases, the regulation protein amino acid sequence is inserted within the RNA effector protein amino acid sequence.

In some cases, the fusion protein comprises a linker and/or a spacer between the RNA effector protein and the regulation moiety.

In some cases, the fusion protein further comprises a nuclear export signal. In some cases, the nuclear export signal is from the HIV Rev protein (LPPLERLTL, SEQ ID NO: 51). In some cases, the nuclear export signal is between the RNA effector protein amino acid sequence and the regulation moiety amino acid sequence.

In some cases, the RNA effector protein is dCas13b and the regulon moiety is PABPC1. Thus in some cases, the fusion protein comprises or consists of SEQ ID NO: 48. In some cases, the fusion protein comprises or consists of SEQ ID NO: 49.

In some cases, the RNA effector protein is dCas13b and the regulon moiety is NAT10.

In some cases, the RNA effector protein and/or regulation moiety is a functionally active portion of an effector protein and/or regulation moiety. Therefore, for example, if the RNA effector protein is C terminal to the regulation moiety, one or more N terminal amino acids of the regulation moiety may be substituted or deleted (e.g., the N terminal methionine). Likewise, if the regulation moiety is C terminal to the RNA effector protein, one or more N terminal amino acids of the RNA effector moiety may be substituted or deleted (e.g., the N terminal methionine).

Also described herein are polynucleotide sequence(s) encoding the fusion protein(s) described herein, vectors comprising the polynucleotide sequence(s), and cells comprising and/or expressing the vector(s).

In some cases, the nucleic acid sequence(s) are codon optimized.

In some cases, the vector(s) comprise a promoter sequence that drives the expression of the fusion protein(s) and/or gRNA(s).

### RNA Effector Proteins

The fusion proteins described herein comprise RNA effector proteins. In some cases, the RNA effector protein is a Cas effector protein or variant or mutant thereof.

In some cases, the RNA effector protein is a Cas9 effector protein (e.g., SEQ ID NO: 46) or a mutant or variant thereof. In some cases, the RNA effector protein is a catalytically inactive Cas9 effector protein, e.g., a Cas9 effector protein with eliminated cleavage activity (e.g., dCas9; e.g., SEQ ID NO: 46 with mutations D10A and H840A).

In some cases, the RNA effector protein is a Cas12 effector protein, e.g., Cas12a (Cpf1), e.g., LbCas12a or a mutant or variant thereof, e.g., a mutant or variant with eliminated cleavage activity. In some cases, the RNA effector protein is a Cas12b effector protein, e.g., AapCas12b or AacCas12b, e.g., a mutant or variant with eliminated cleavage activity.

In some cases, the RNA effector protein is Cas14 effector protein or a mutant or variant thereof. See, e.g., Harrington et al., “Programmed DNA Destruction by Miniature CRISPR-Cas13 Enzymes,” Science 362 (6146): 839-42 (2018); see also Karvelis et al., “PAM Recognition by Miniature CRISPR-Cas12f Nucleases Triggers Programmable Double-Stranded DNA Target Cleavage,” Nucleic Acids Res 48 (9): 5016-23 (2020).

Type VI CRISPR-Cas systems contain the programmable single-effector RNA-guided RNases Cas13. See, e.g., Cox et al., “RNA Editing with CRISPR-Cas13,” Science 358 (6366): 1019-27 (2017). The Cas13 family contains at least four known subtypes, including Cas13a (formerly C2c2), Cas13b, Cas13c, and Cas13d.

In some cases, the RNA effector protein is a Cas13 effector protein, e.g., Cas13a, Cas13b, Cas13c, or Cas13d. In some cases, the RNA effector protein is a Cas13b effector protein. See, e.g., Smargon et al. (2017), “Cas13b Is a Type VI-B CRISPR-Associated RNA-Guided RNase Differentially Regulated by Accessory Proteins Csx27 and Csx28,” Molecular Cell 65, 618-630 (Feb. 16, 2017); see also Smargon et al., “RNA-Targeting CRISPR Systems from Metagenomic Discovery to Transcriptome Engineering,” Nat Cell Biol 22 (2): 143-50 (2020).

In some cases, the RNA effector protein is a catalytically inactive RNA-effector protein, e.g., a Cas13 effector protein with eliminated cleavage activity (dCas13), e.g., dCas13b (SEQ ID NO: 47)).

In some cases, the RNA effector protein has at least 80%, e.g., at least 85%, 90%, 95%, 98%, or 100% sequence identity compared to SEQ ID NO: 47.

In some cases, the Cas13 effector protein is a Cas13bt. See, e.g., Kannan et al., “Compact RNA Editors with Small Cas13 Proteins,” Nature Biotechnology 18:499-560 (2021). In some cases, the Cas13 effector protein is a catalytically inactive Cas13bt effector protein (dCas13bt). In some cases, the Cas13 effector protein is a Cas13bt with mutations corresponding to H133A and H1058 of the dCas13bt.

In some cases, the Cas13 effector protein is a Cas13 effector protein described in WO2018170333A1 (e.g., in Table 1A of WO2018170333A1). In some cases, the Cas13 is a catalytically inactive mutant of a Cas13 described in WO2018170333Al (e.g., a mutant of a Cas13 shown in Table 1A of WO2018170333A1).

### Regulon Moieties

The fusion proteins described herein comprise regulation moieties. In some cases, the regulation moiety that stimulates and/or stabilizes an mRNA, e.g., an mRNA of an active allele of a gene associated with a haplotype insufficiency disorder. In some cases, the regulation moiety stimulates activity of the mRNA. In some cases, the regulation moiety stabilizes the mRNA.

In some cases, the regulation moiety can be a translational stimulator protein. In some cases, the regulation moiety can be a wild-type protein. In some cases, the regulation moiety can be a truncated variant of a wild-type protein.

### PABPC1

In some cases, the regulation moiety is PABPC1. Polyadenylate-binding protein 1 PABPC1 is a highly conserved RNA binding protein in eukaryotes. This protein has four N-terminal RNA recognition motif (RRM) domains, which bind poly(A) RNA with a nanomolar affinity [23, 24]. The RRMs are followed by a proline-rich linker and a C-terminal MLLE domain. The MLLE domain recognizes a peptide motif called poly(A)-interacting motif 2 (PAM2), which is found in a number of PABPC partner proteins that regulate mRNA metabolism (stability and translation). The presence of PABPC1 on mRNA is known to stimulate their activity, enhancing translation and mRNA stability [19].

Thus, in some cases, the regulation moiety comprises or consists of SEQ ID NO: 42 or SEQ ID NO: 43.

In some cases, the regulation moiety comprises or consists of polypeptide sequence having at least 80%, e.g., at least 85%, at least 90%, at least 95%, or at least 99% sequence identity to SEQ ID NO: 42 or SEQ ID NO: 43.

In some cases, the regulation moiety comprises a wild-type PABPC1 protein. In some cases, the regulation moiety comprises a truncated variant of the wild-type PABPC1 protein. In some embodiments, the regulation moiety comprises the RRM domains and the MLLE domains. In some case, the regulation moiety comprises the MLLE domain.

### NAT10

In some cases, the regulation moiety is NAT10. RNA cytidine acetyltransferase NAT10 is a highly conserved enzyme that catalyzes the conversion of cytidine to N4-acetylcytidine (ac4C) [25]. Generation of the “epitranscriptome” through post-transcriptional ribonucleoside modification embeds a layer of regulatory complexity into RNA structure and function. N4-acetylcytidine mRNA modification has been established to have robust stimulatory effect on mRNA stability and translation in human cells [25].

Thus, in some cases, the regulation moiety comprises or consists of SEQ ID NO: 44 or SEQ ID NO: 45. In some cases, the regulation moiety comprises or consists of a polypeptide sequence having at least 80%, e.g., at least 85%, at least 90%, at least 95%, or at least 99% sequence identity to SEQ ID NO: 44 or SEQ ID NO: 45.

### Pharmaceutical Compositions and Methods of Treatment

### Pharmaceutical Compositions

Also described herein are pharmaceutical compositions comprising a fusion protein described herein, pharmaceutical compositions comprising a nucleic acid encoding the fusion proteins described herein, and pharmaceutical compositions comprising a vector comprising a nucleic acid encoding a fusion protein described herein.

In some cases, the pharmaceutical composition further comprises a gRNA, e.g., as described herein. In some cases, the pharmaceutical composition further comprises a nucleic acid encoding a gRNA, e.g., as described herein. In some cases, the pharmaceutical composition further comprises a vector comprising a nucleic acid encoding a gRNA, e.g., as described herein.

In some cases, the pharmaceutical composition is formulated for gene therapy, e.g., as described herein.

### Fusion Proteins

In some cases, the pharmaceutical compositions described herein comprise a fusion protein, e.g., a fusion protein described herein. In some cases, the pharmaceutical compositions described herein comprise a nucleic acid encoding a fusion protein, e.g., a fusion protein described herein. In some cases, the pharmaceutical composition comprises the nucleic acid encoding a fusion protein, e.g., a fusion protein described herein.

gRNAS

In some cases, e.g., when the fusion protein comprises a Cas effector protein, e.g., as described herein, the pharmaceutical composition further comprises a guide RNA (gRNA) comprising a complementarity region designed to complex with the Cas protein and hybridize to a nucleic acid, e.g., an mRNA of an active allele of a gene associated with a haploinsufficiency disorder, e.g., the 3′ UTR of an mRNA of an active allele of a gene associated with a haploinsufficiency disorder.

In some cases, the gRNA is designed to hybridize to the 3′ UTR of an mRNA. In some cases, the gRNA can hybridize to a 3′ end of the 3′ UTR transcript. In some cases, the gRNA can hybridize to a position within the 3′ UTR transcript.

In some cases, the gRNA comprises a CRISPR RNA (crRNA). In some cases, the gRNA comprises a trans-activating CRISPR RNA (tracrRNA). In some cases, the gRNA is a single guide RNA (sgRNA). In some cases, the gRNA does not comprise a tracrRNA.

In some cases, the gRNA is designed to complex with Cas13b and comprises a complementary region designed to hybridize to an mRNA of an active allele of a gene associated with a haploinsufficiency disorder, e.g., the 3′ UTR of an mRNA of an active allele of a gene associated with a haploinsufficiency disorder.

In some cases, the Cas13b is dCas13b (SEQ ID NO: 47).

In some cases, the gRNA comprises a complementarity region designed to hybridize to an mRNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof. In some cases, the gRNA comprises a complementarity region designed to hybridize to an mRNA selected from the group consisting of SEQ ID NO: 4, SEQ ID NO: 6, SEQ ID NO: 10, SEQ ID NO: 15, SEQ ID NO: 17, and combinations thereof.

In some cases, the gRNA comprises a complementarity region designed to hybridize to an mRNA encoding an amino acid selected from the group consisting of SEQ ID NO: 19, SEQ ID NO: 20, SEQ ID NO: 21, SEQ ID NO: 22, SEQ ID NO: 23, SEQ ID NO: 24, and combinations thereof. In some cases, the gRNA comprises a complementarity region designed to hybridize to an mRNA encoding SEQ ID NO: 21.

In some cases, the gRNA comprises a complementarity region designed to hybridize to an mRNA selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof. In some cases, the gRNA comprises a complementarity region designed to hybridize to SEQ ID NO: 26.

In some cases, the gRNA comprises a complementarity region designed to hybridize to an mRNA encoding SEQ ID NO: 37, SEQ ID NO: 38, SEQ ID NO: 39, SEQ ID NO: 40, SEQ ID NO: 41, and combinations thereof. In some cases, the gRNA comprises a complementarity region designed to hybridize to an mRNA encoding SEQ ID NO: 37.

In some cases, the gRNA targets an mRNA encoding Mecp2. In some cases, the gRNA targets an mRNA encoding SynGAP. In some cases, the gRNA targets an mRNA encoding SHANK3. In some cases, the gRNA targets an mRNA encoding PTEN. In some cases, the gRNA targets an mRNA encoding CHD2. In some cases, the gRNA comprises or consists of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, or SEQ ID NO: 72.

A list of gRNAs to target the 3′ UTR of genes of interest is shown in Table 7.

TABLE 7
List of guide RNAs to target the 3′UTR of the genes of interest.

| | | | SEQ ID |
|---|---|---|---|
| Name | Target Gene | Sequence | NO: |
| oJC5003 | pJC889- | CACCGCACTATAGAATAGGGCCCTCTAG | 55 |
| | gRNA1 | | |
| oJC5005 | pJC889- | CACCGGAGCTCTAGCATTTAGGTGACAC | 56 |
| | gRNA2 | | |
| oJC5051 | Mecp2-G1 | CACCGAAACACTTAGAGTTTCGGAGCTT | 57 |
| oJC5053 | Mecp2-G2 | CACCGTGCCATTCAAGAAGAACTCCGAG | 58 |
| oJC5055 | Mecp2-G3 | CACCGATTACACCATAGTAAGGAAGCCAGG | 59 |
| oJC5057 | Mecp2-G4 | CACCGAAATTCACTGAAAGAGGCCCCCA | 60 |
| oJC5059 | Mecp2-G5 | CACCGACAGAAGATTGTCTGCACCCGCA | 61 |
| oJC5061 | Mecp2-G6 | CACCGGCTAAAAATGTATATGCCCAAAG | 62 |
| oJC5063 | Mecp2-G7 | CACCGCCAAAAGACAGGTTCAGAAGCCA | 63 |
| oJC5241 | SynGAP-G1 | CACCGCACCGAAGTTGAAAGTTTGGAGGTGCCA | 64 |
| oJC5243 | SynGAP-G2 | CACCGCACCGACCCCAGATAATTACAACAGCCA | 65 |
| oJC5556 | SHANK3-G1 | CACCGCACACACATAAAGGGCAGGACAG | 66 |
| oJC5558 | SHANK3-G2 | CACCGAAACCCAAATTGAACGGAACCAA | 67 |
| oJC5560 | PTEN-G1 | CACCGAAGACAACTGATAAGCCAACAGC | 68 |
| oJC5562 | PTEN-G2 | CACCGAGCCAGTAAATGATAGGCTACAG | 69 |
| oJC5564 | CHD2-G1 | CACCGGAATAAAACTGAAGGCCCCAGCA | 70 |
| oJC5566 | CHD2-G2 | CACCGCAGTAGAAACTTGTTCACCACAG | 71 |
| oJC5568 | CHD2-G3 | CACCGTACAAGAATCAAAAGTACAGCAG | 72 |

Also provided herein are polynucleotide sequences encoding the gRNA(s) described herein, vectors comprising the polynucleotide sequence(s) encoding the gRNA(s) described herein, and cells comprising the vector(s) encoding the gRNA(s) described herein.

In some cases, the polynucleotide encoding the fusion protein and the polynucleotide encoding the gRNA are on the same vector. In some cases, the polynucleotide encoding the fusion protein and the polynucleotide encoding the gRNA are on different vectors.

### Gene Therapy

The nucleic acids described herein, e.g., nucleic acids encoding fusion protein(s) and/or gRNA(s) described herein, can be incorporated into a gene construct to be used as a part of a gene therapy protocol. Thus, also provided herein are targeted expression vectors for in vivo transfection and expression of a polynucleotide that encode fusion protein(s) and/or gRNA(s) described herein. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In addition to viral transfer methods, such as those illustrated above, non-viral methods can also be employed to cause expression of a nucleic acid compound described herein, e.g., nucleic acid compound(s) encoding the fusion protein(s) and/or gRNA(s) described herein. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, lipid nanoparticles and artificial viral envelopes. Other embodiments include plasmid injection systems such as are described in Meuli et al., J. Invest. Dermatol. 116 (1): 131-135 (2001); Cohen et al., Gene Ther. 7 (22): 1896-905 (2000); or Tam et al., Gene Ther. 7 (21): 1867-74 (2000).

In some embodiments, nucleic acid compound described herein, e.g., nucleic acid compound(s) encoding the fusion protein(s) and/or gRNA(s), is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91:3054-3057 (1994)).

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Treatment

The methods described herein include methods for the treatment of disorders associated with haploinsufficiency, e.g., as described herein. In Generally, the methods include administering a therapeutically effective amount of a pharmaceutical composition as described herein, to a subject who is in need of, or who has been determined to be in need of, such treatment, e.g., but a gene therapy method described herein.

In some cases, the methods of treatment provided herein may be used to treat a subject (e.g., human, monkey, dog, cat, mouse) who has been diagnosed with or is suspected of having a haploinsufficiency disorder, e.g., as described herein. In some embodiments, the subject is a mammal. In some embodiments, the subject is a human.

In some cases, the subject may be a human who exhibits one or more symptoms associated with a haploinsufficiency disorder, e.g., as described herein. Any of the methods of treatment provided herein may be used to treat haploinsufficiency disorders at various stages.

In some cases, the disorder is Dravet Syndrome. In some cases, the subject has a mutation selected from the list in Table 4.

In some cases, the disorder is Rett Syndrome. In some cases, the subject has a mutation selected from the list in Table 6.

As used in this context, to “treat” means to ameliorate at least one symptom of the disorder associated with a haploinsufficiency disorder. Often, a haploinsufficiency disorder results in the amount of gene product expressed from mRNA(s) of the active allele being insufficient for proper gene function; thus, a treatment can result in an increase in the amount of gene product expressed from mRNA(s) of an active allele as compared to, e.g., an untreated age-matched subject.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Variants

In some cases, the fusion protein(s) described herein, or the nucleic acid sequences encoding them, have at least 80%, e.g., at least 85%, 90%, 95%, 98%, or 100% identity to the amino acid sequence of a sequence provided herein, e.g., has differences at up to 1%, 2%, 5%, 10%, 15%, or 20% of the residues of the sequence provided herein replaced, e.g., with conservative mutations, e.g., including or in addition to the mutations described herein. In preferred embodiments, the variant retains desired activity of the parent, e.g., the binding activity of the RNA-effector protein and the regulation activity of the regulon moiety.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215:403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### EXAMPLES

The invention is further described in the following examples, which do not limit the scope of the invention described in the claims.

### Example 1: PAB Tethered Targeting

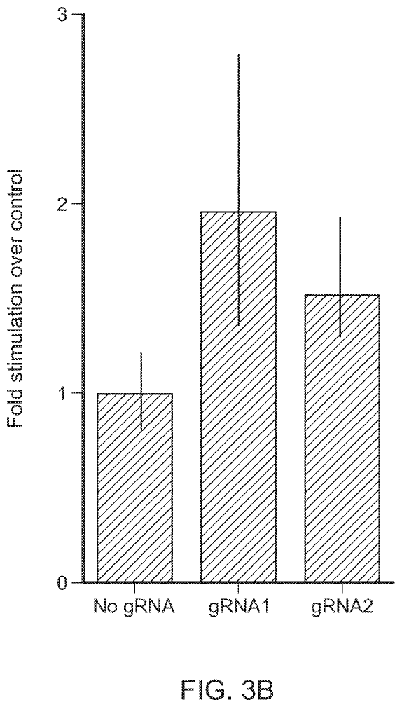

FIG. 3A is a schematic showing the experimental approach applied for this assay. Briefly, PABPC1 was fused to dCas13b tethering protein and guided to 3′UTR of the Luciferase gene via designed guide RNAs (gRNA) to enhance its translation.

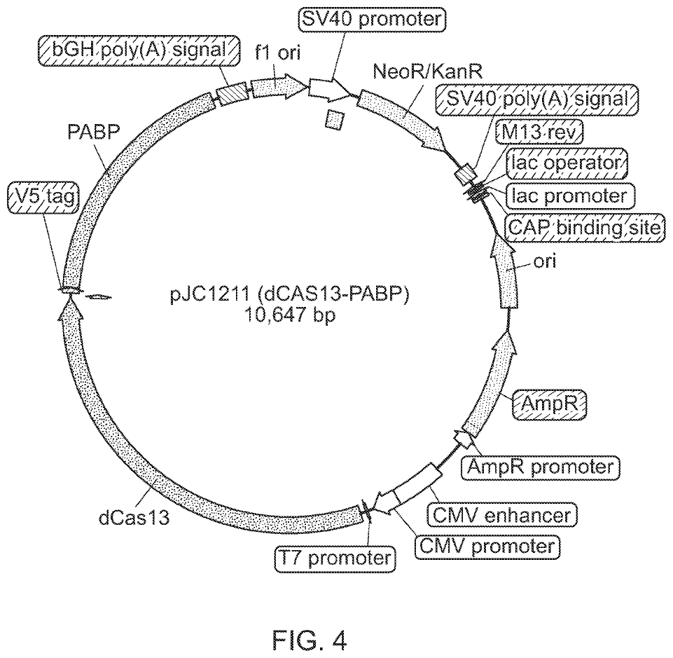

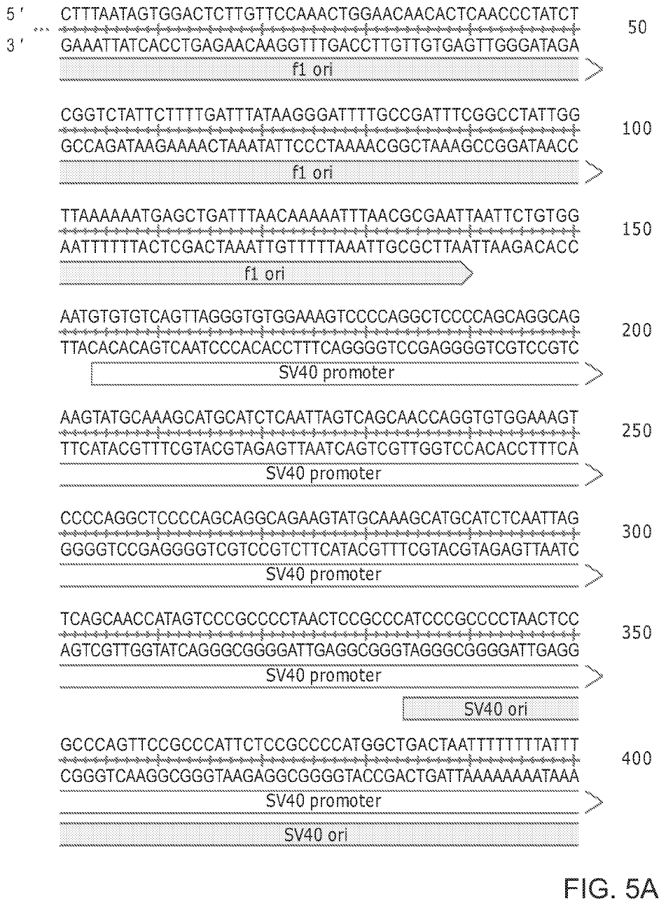

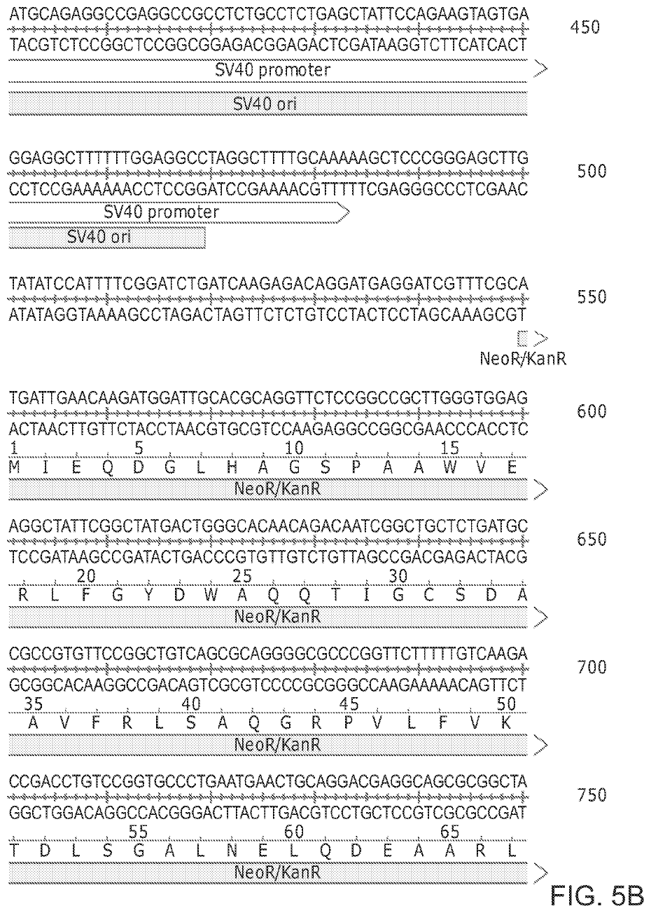

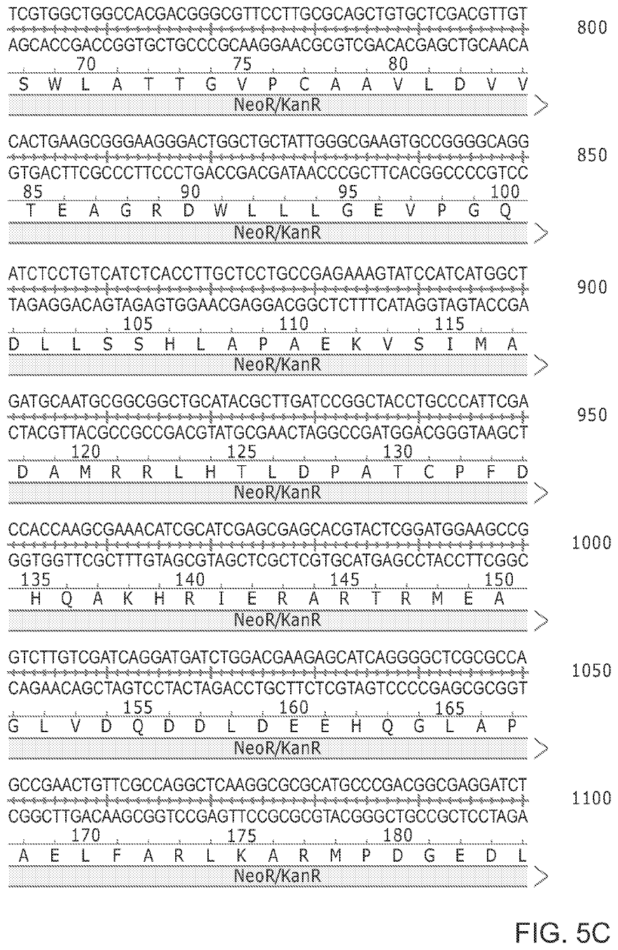

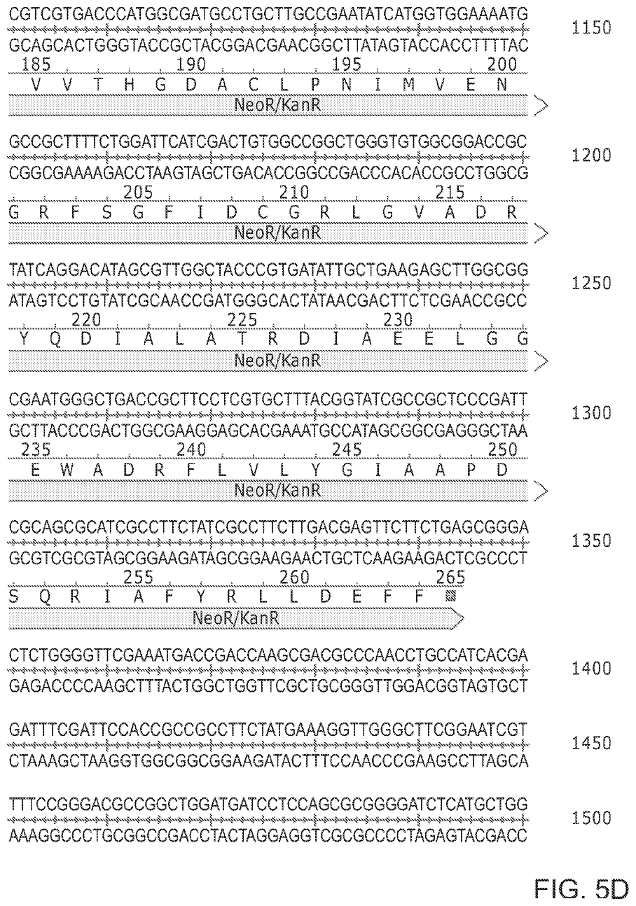

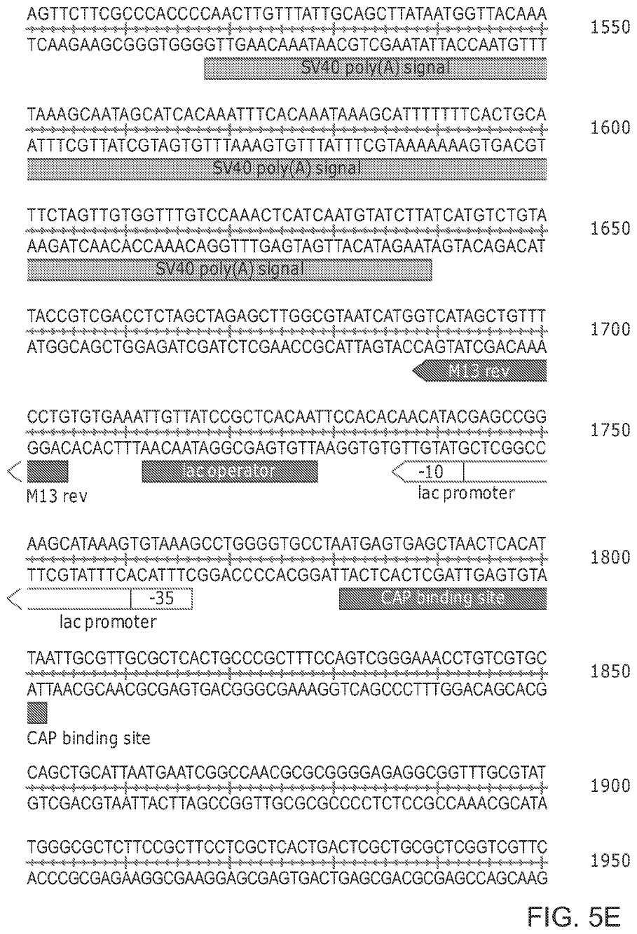

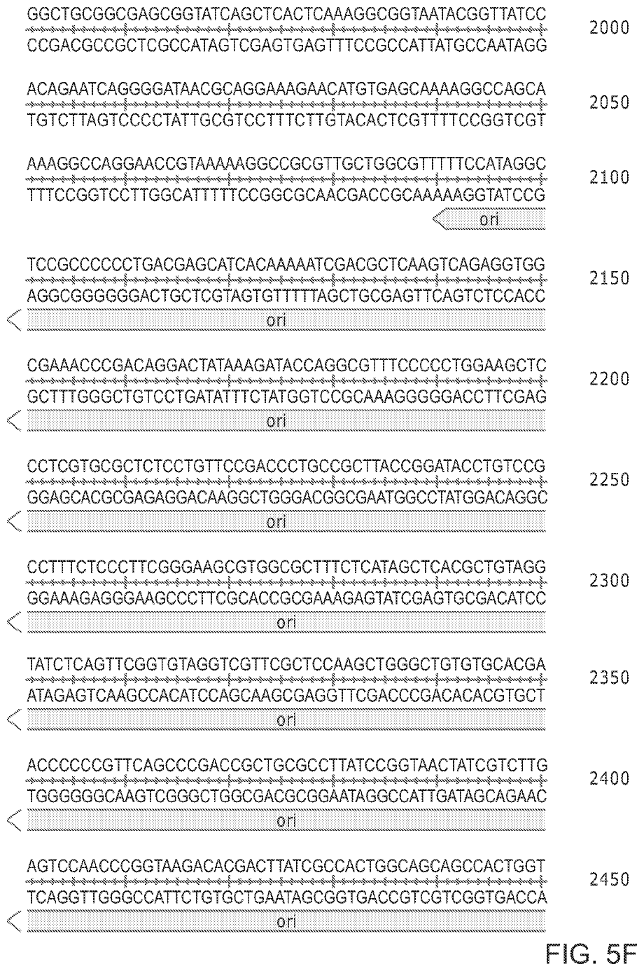

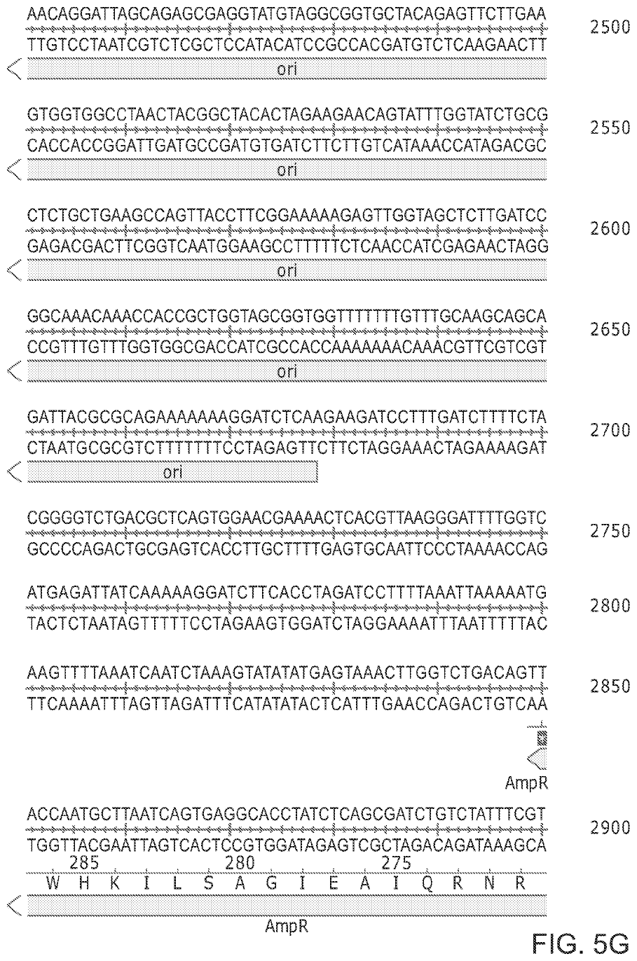

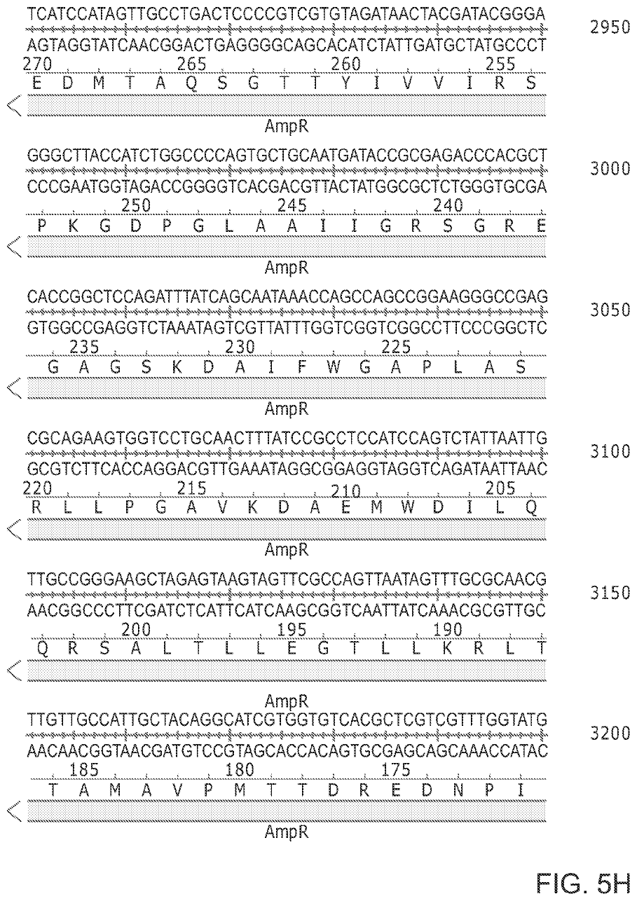

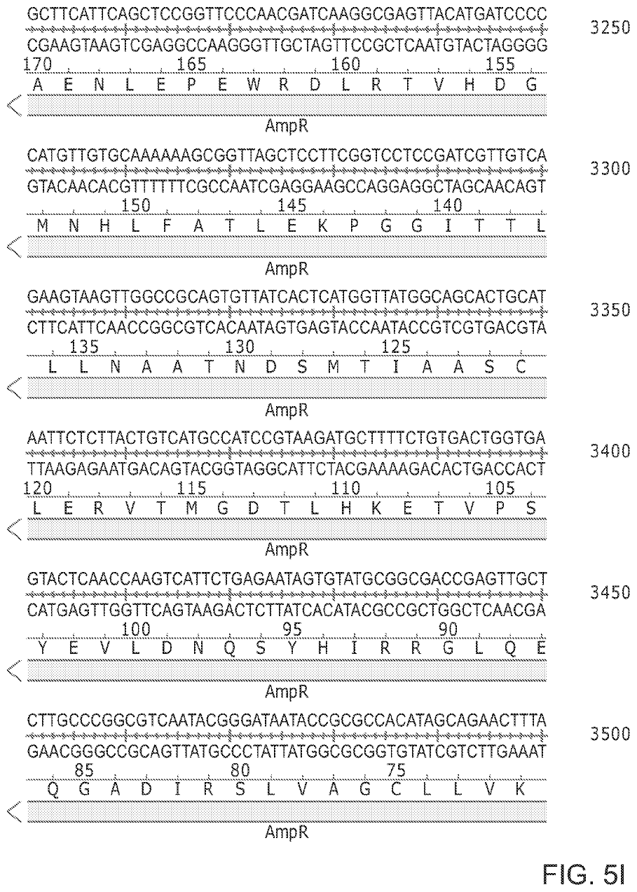

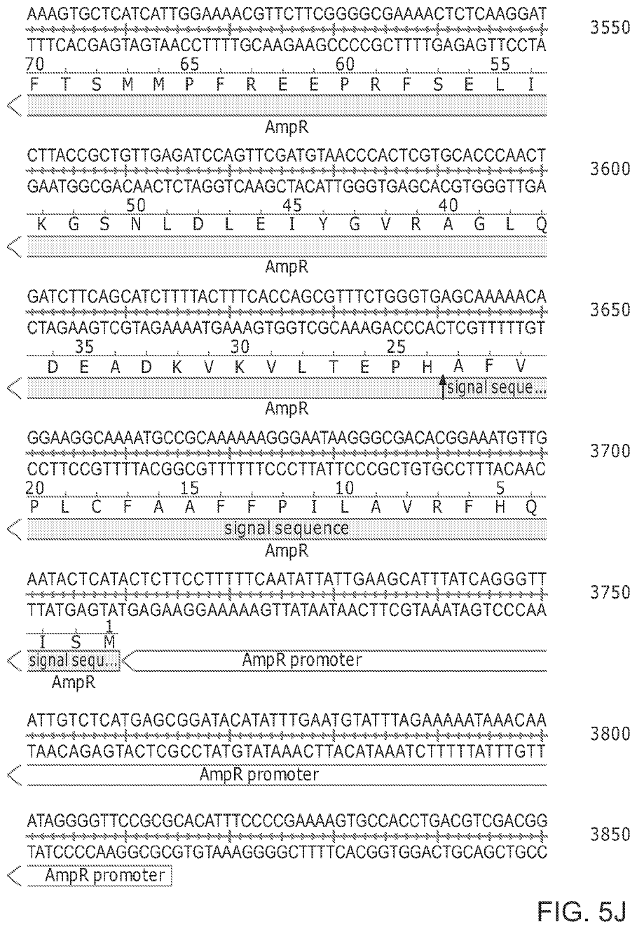

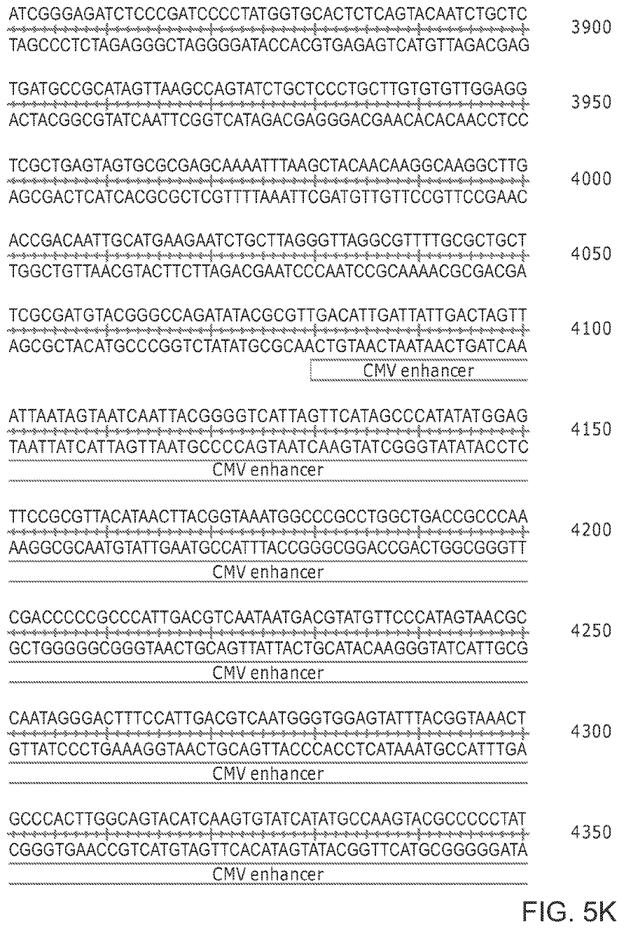

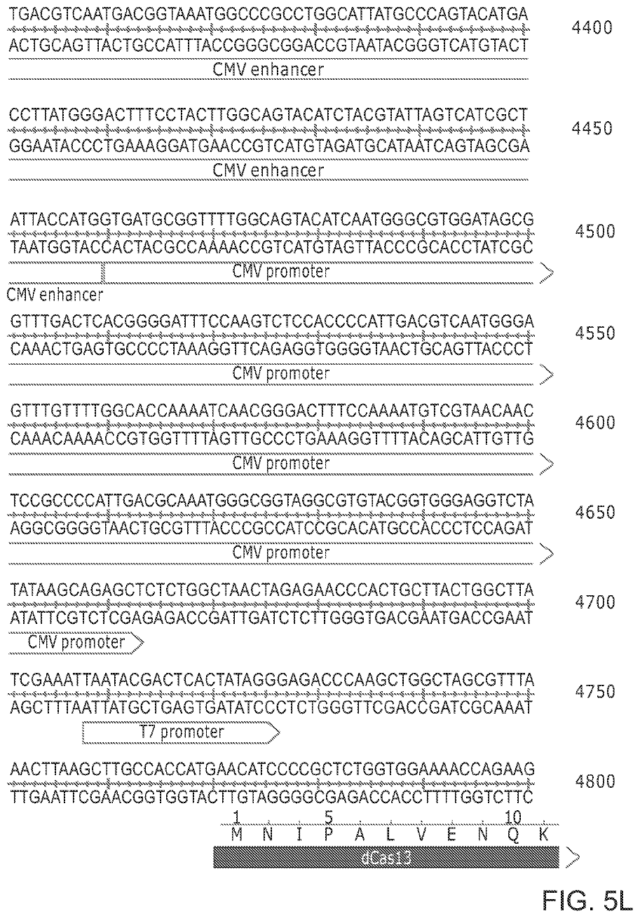

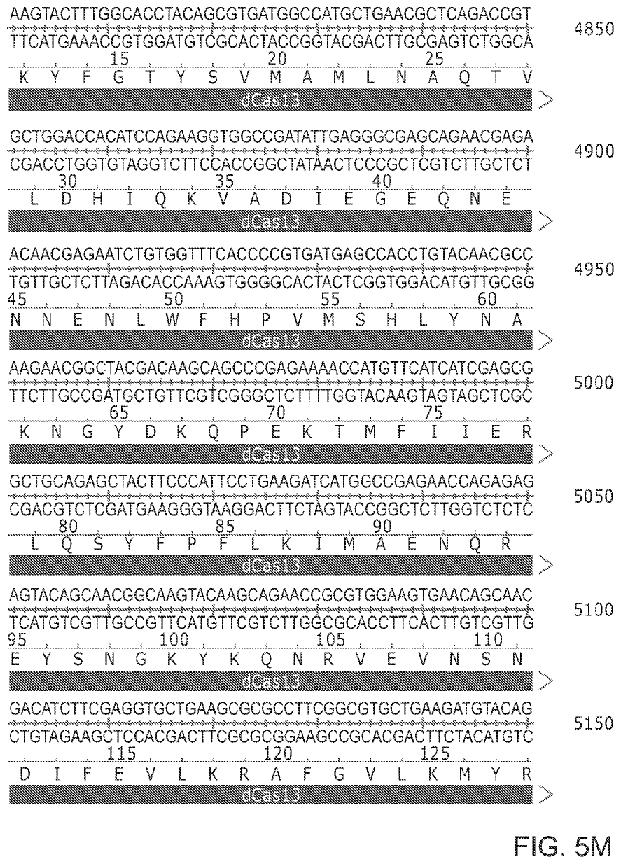

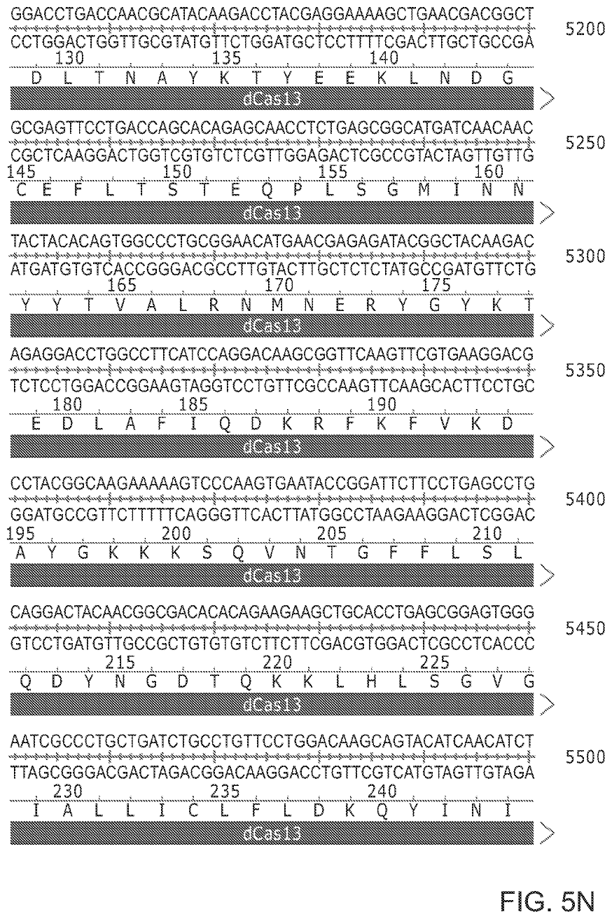

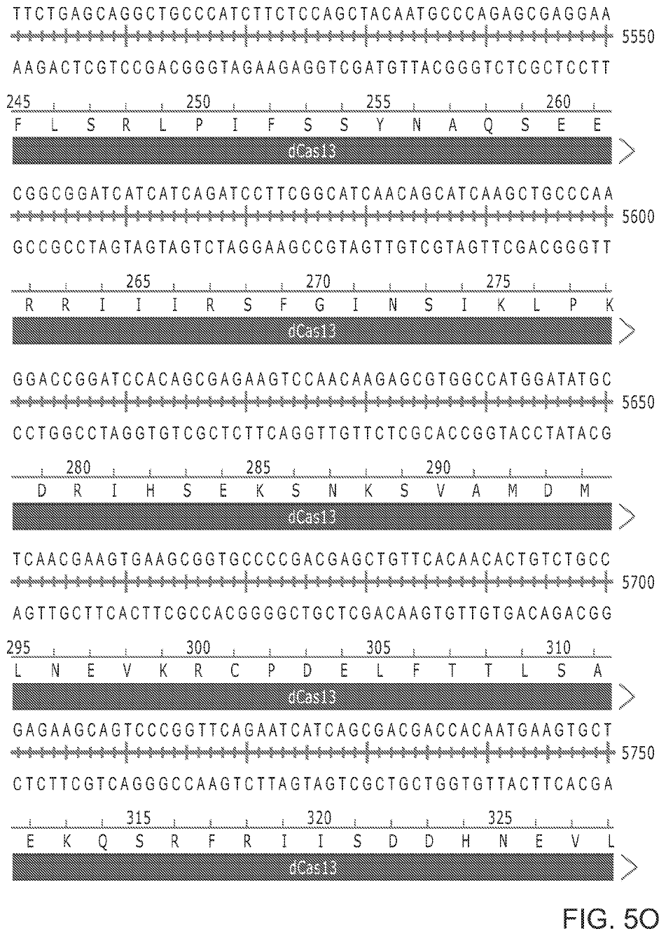

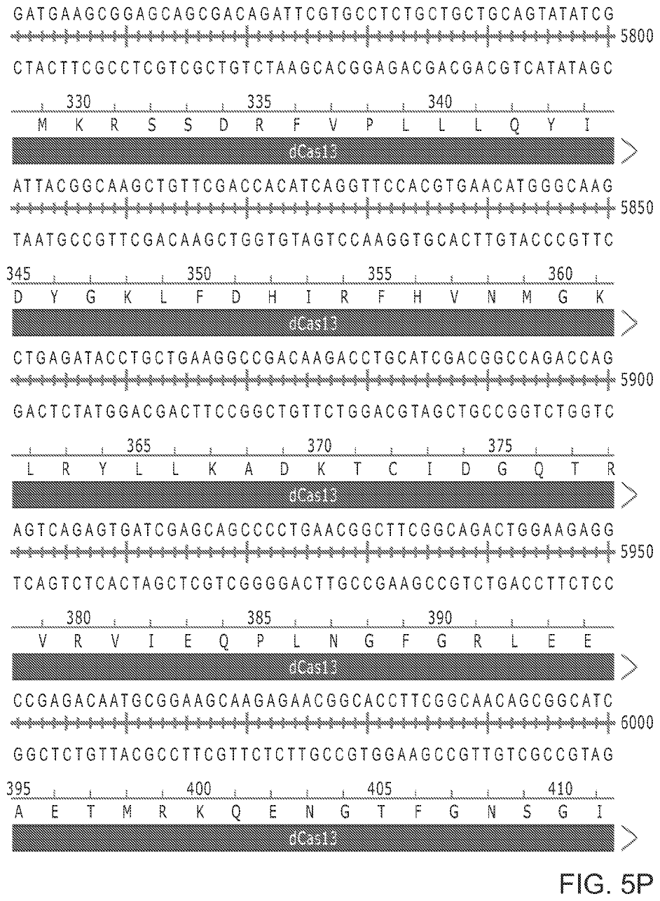

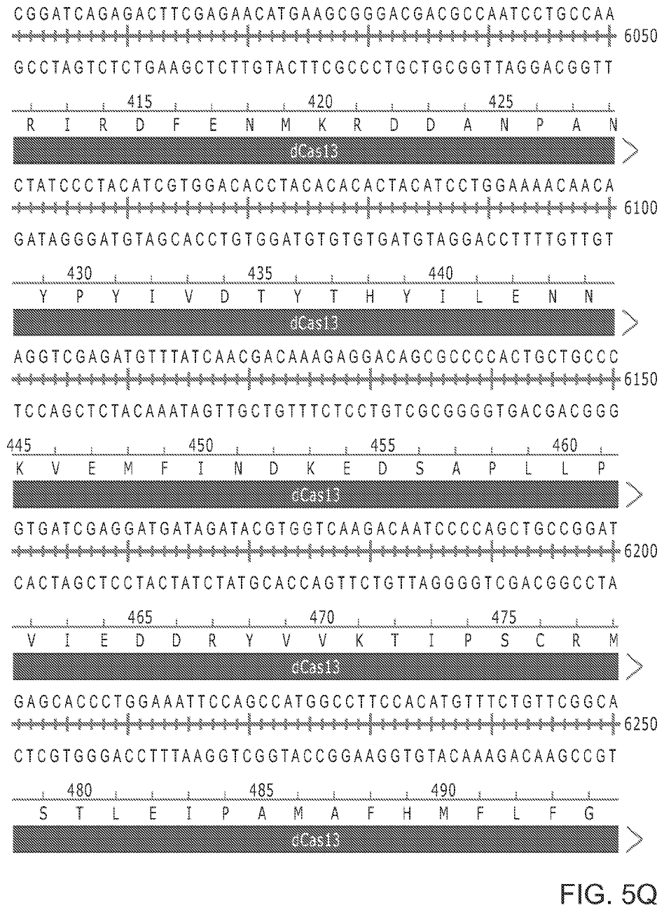

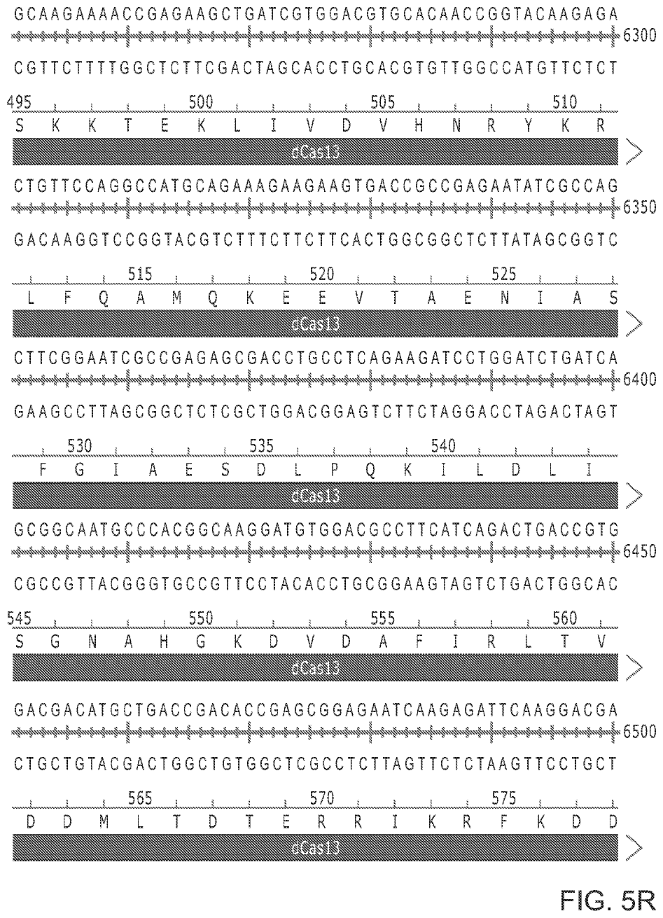

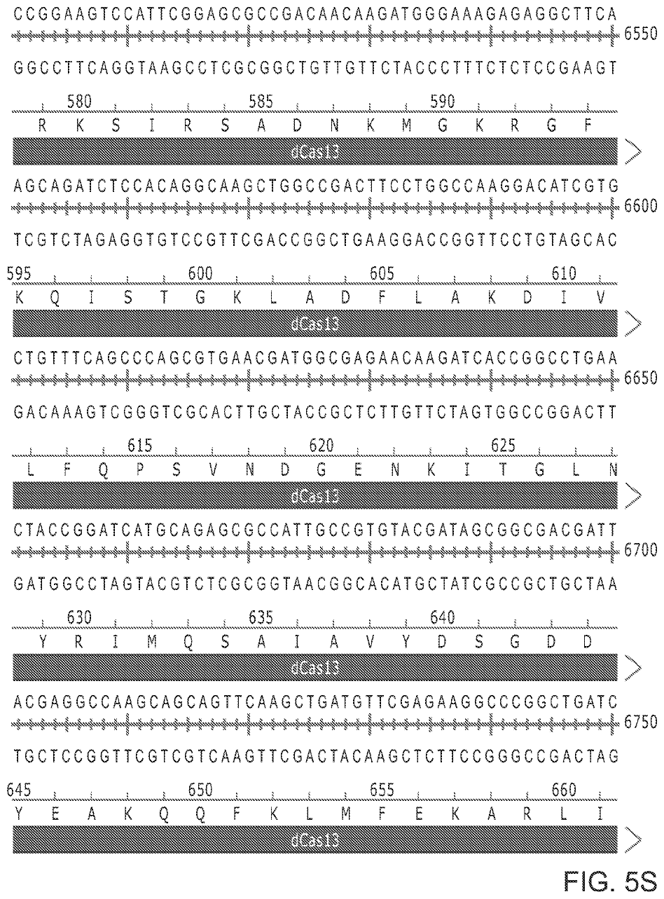

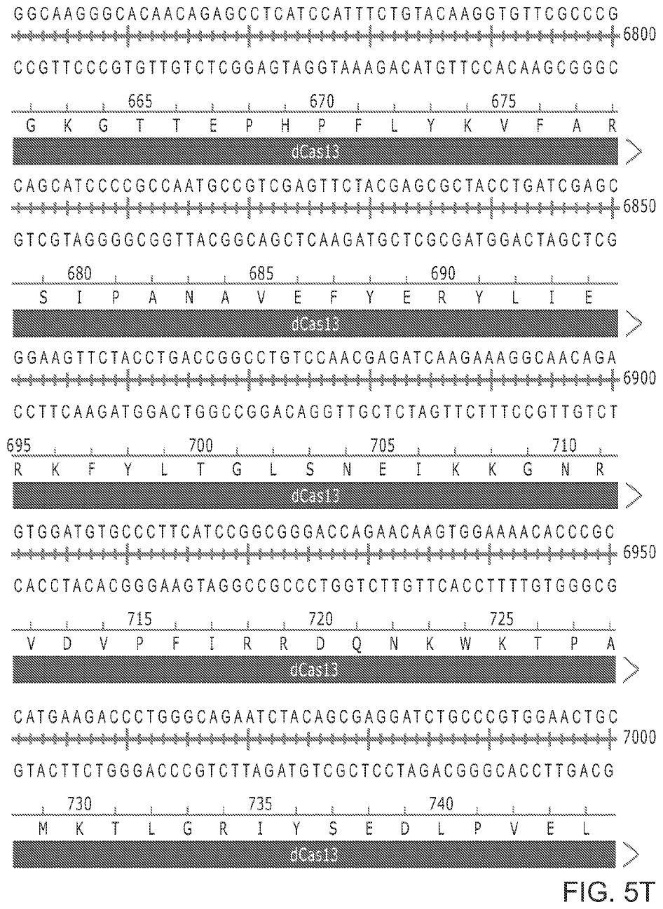

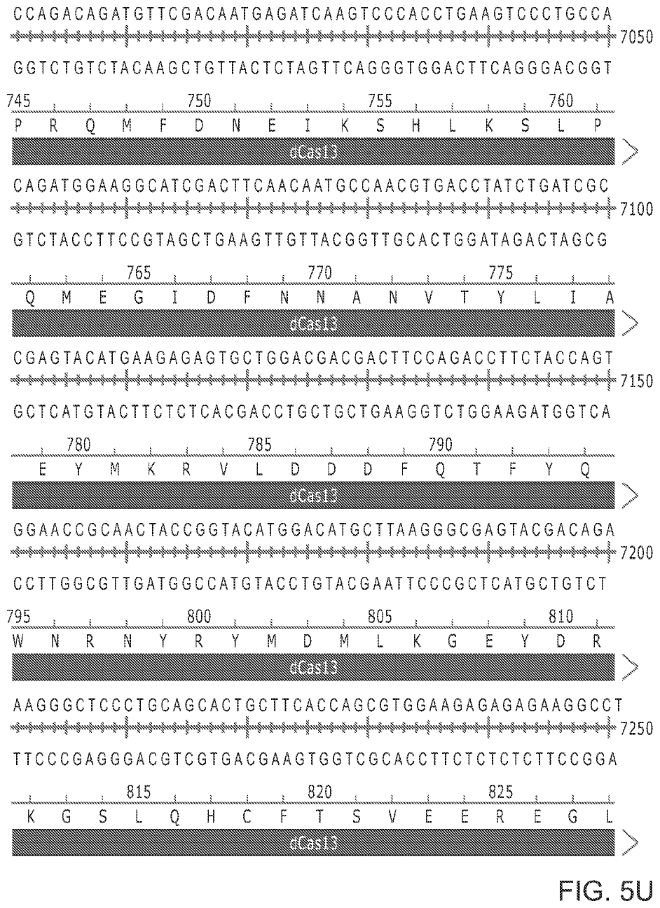

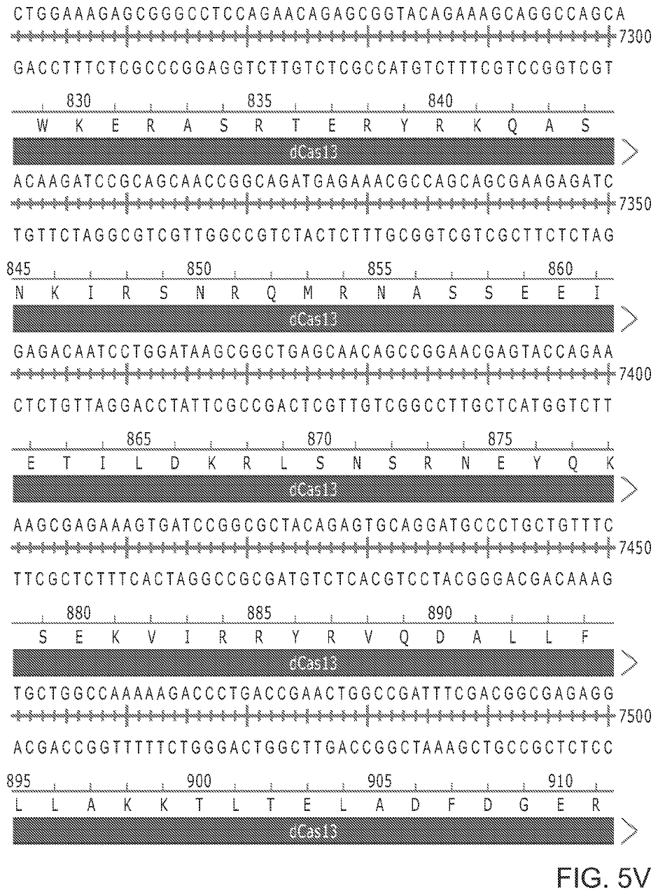

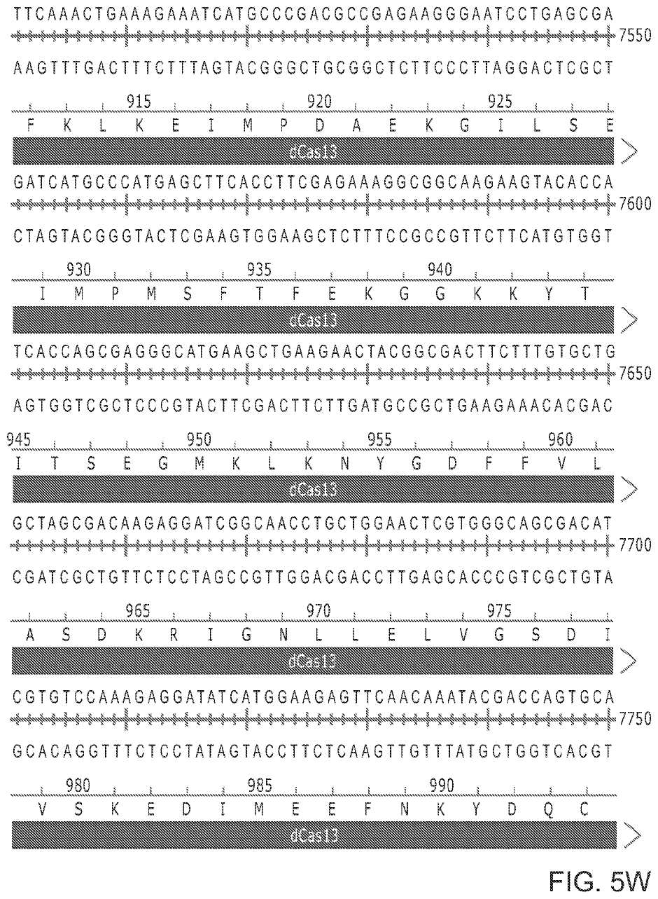

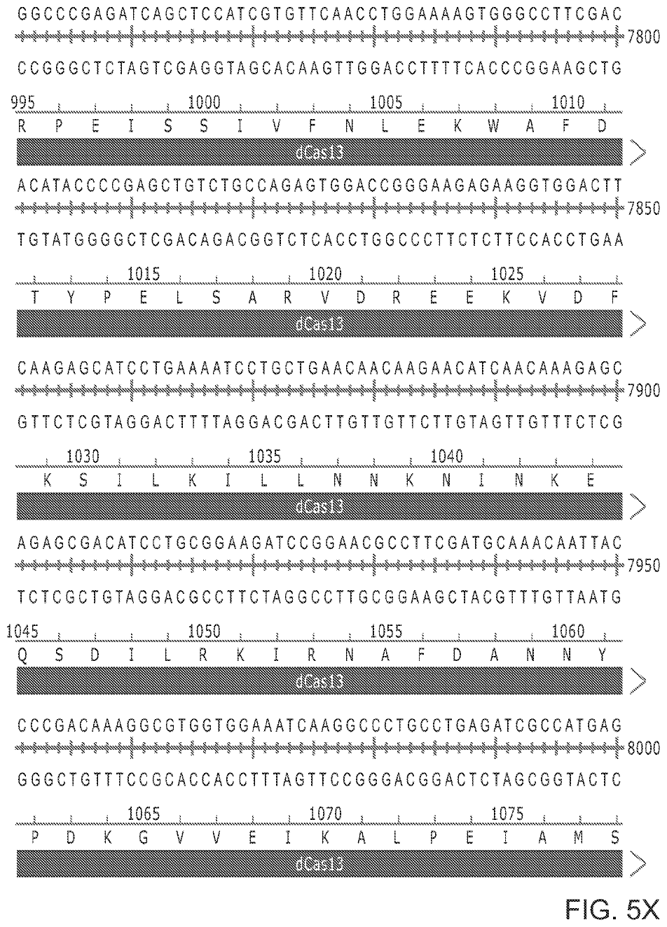

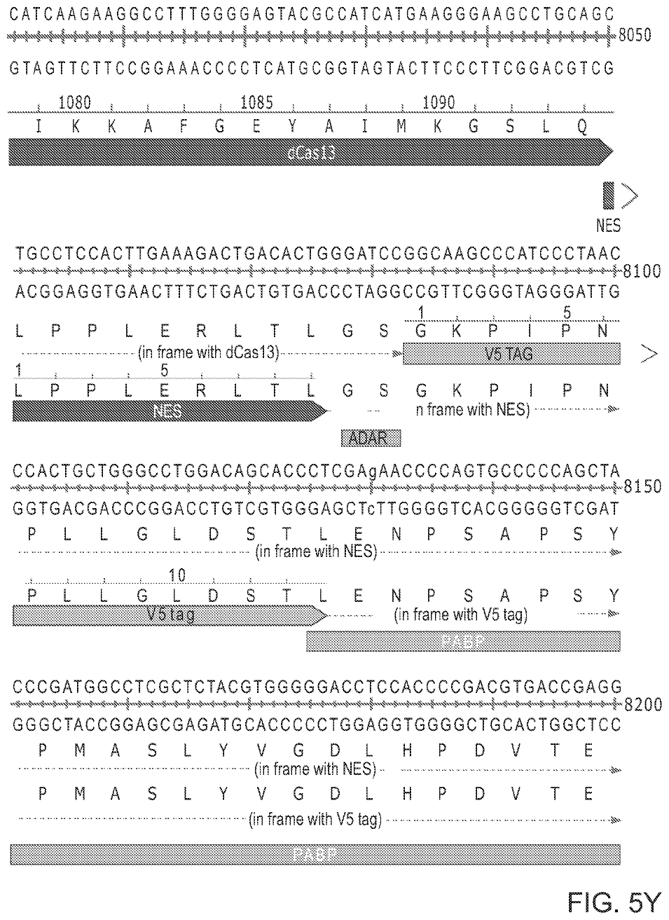

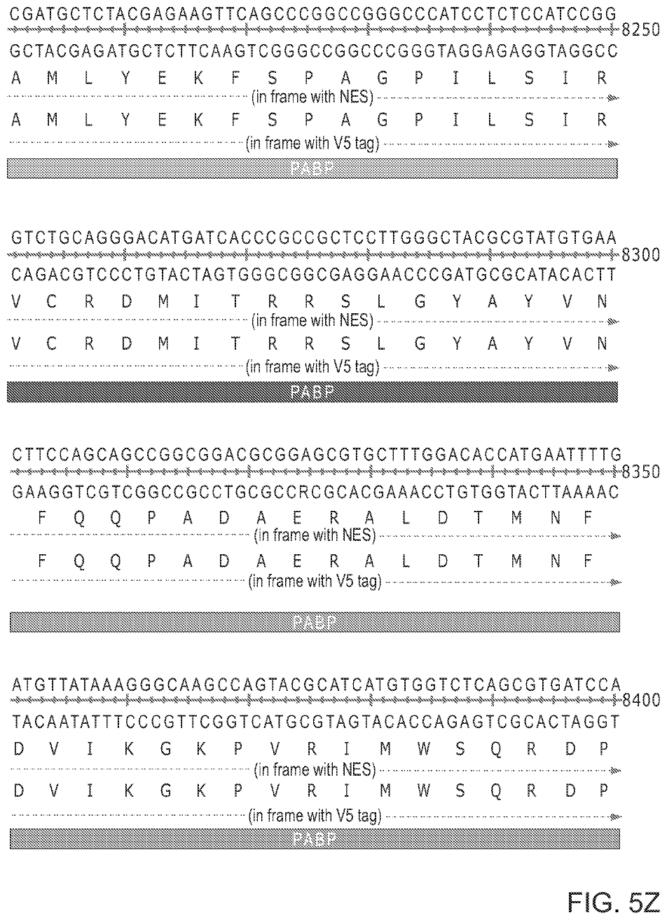

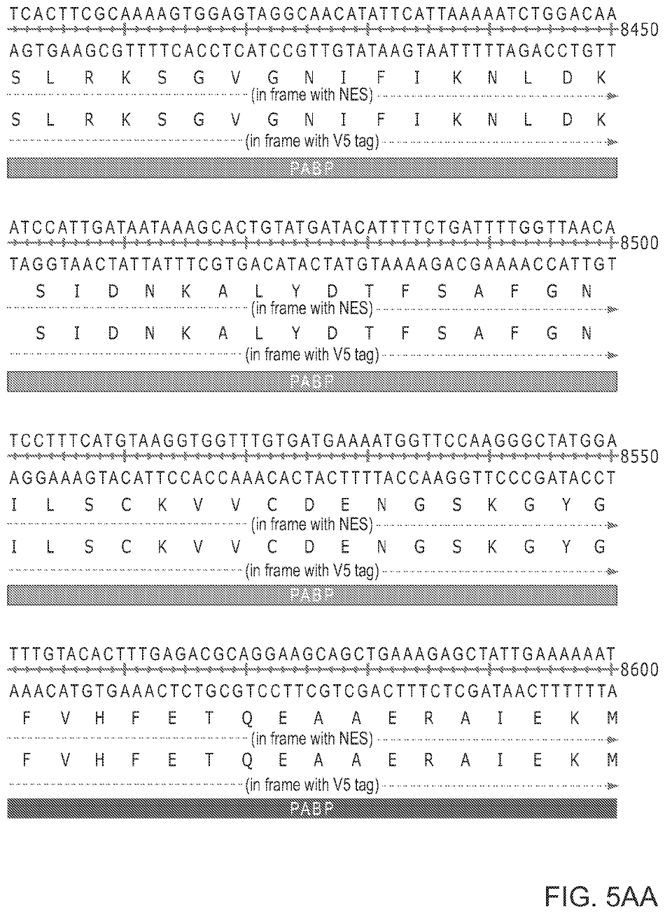

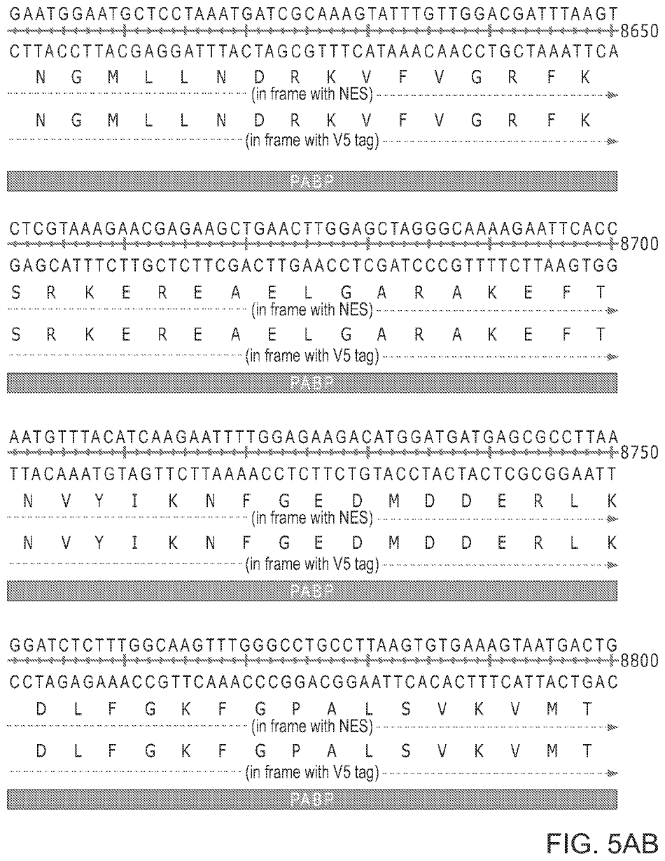

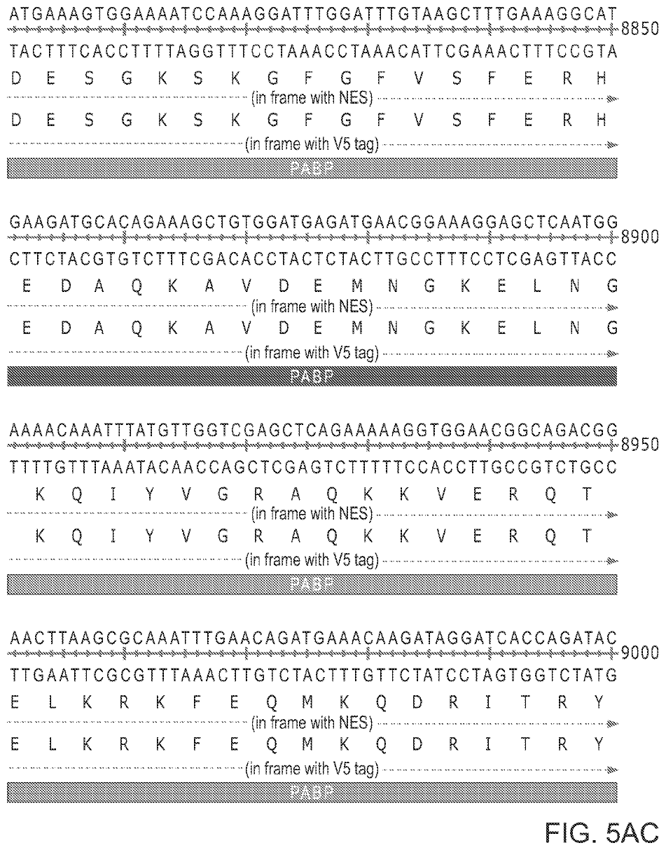

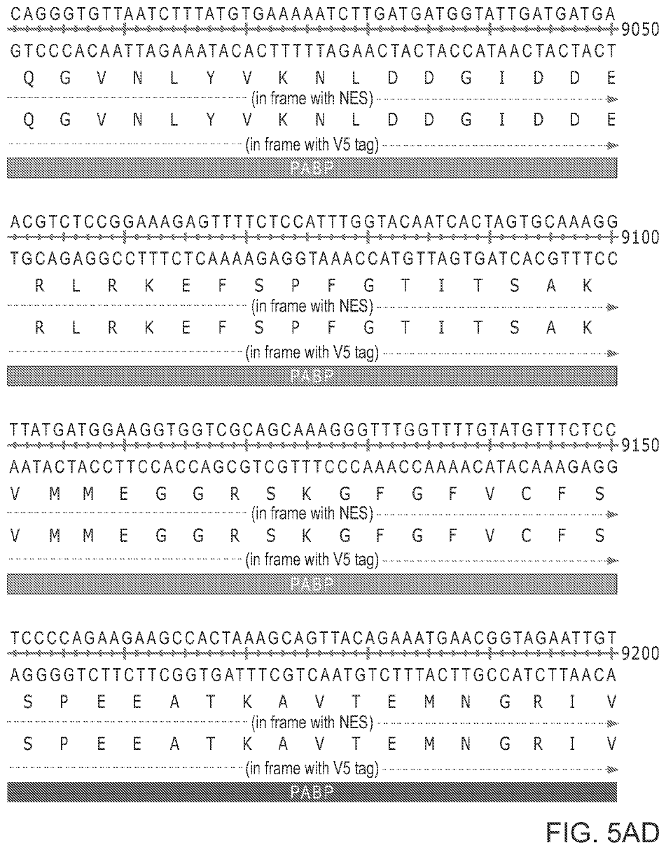

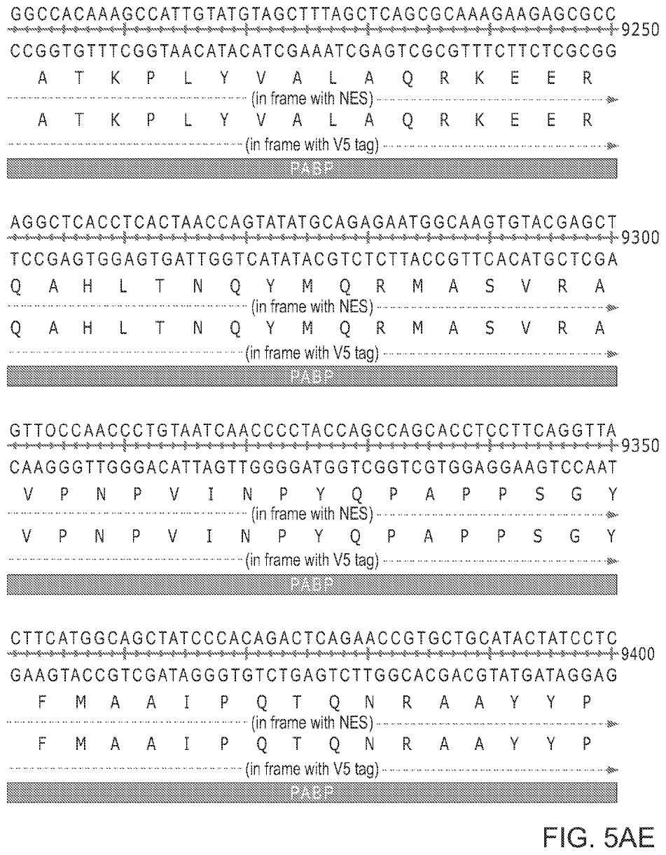

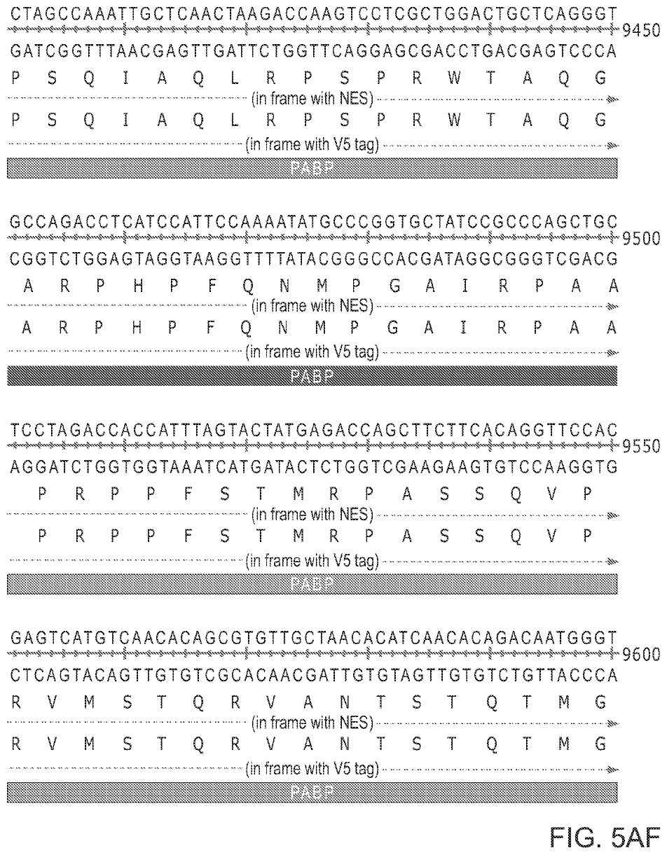

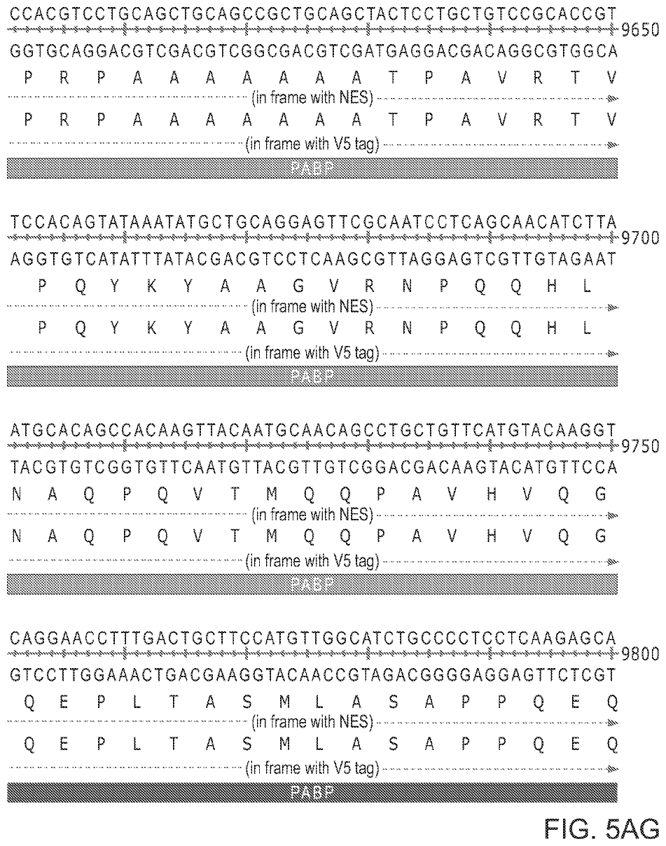

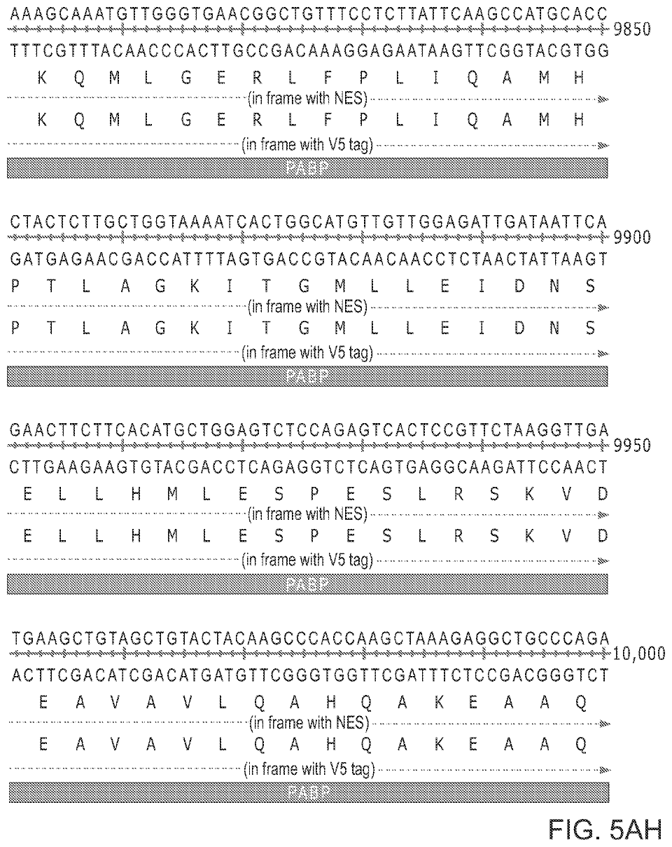

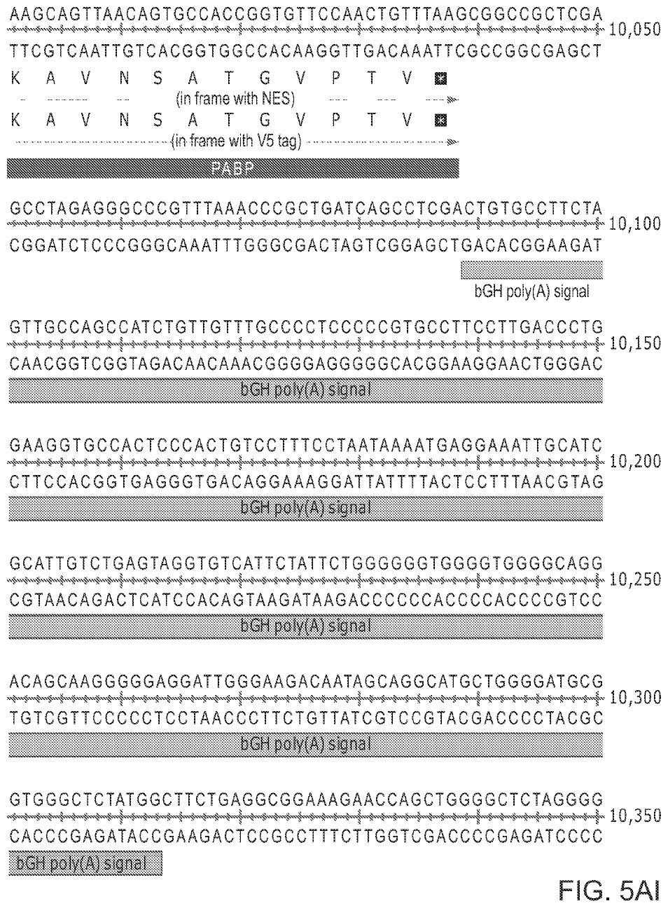

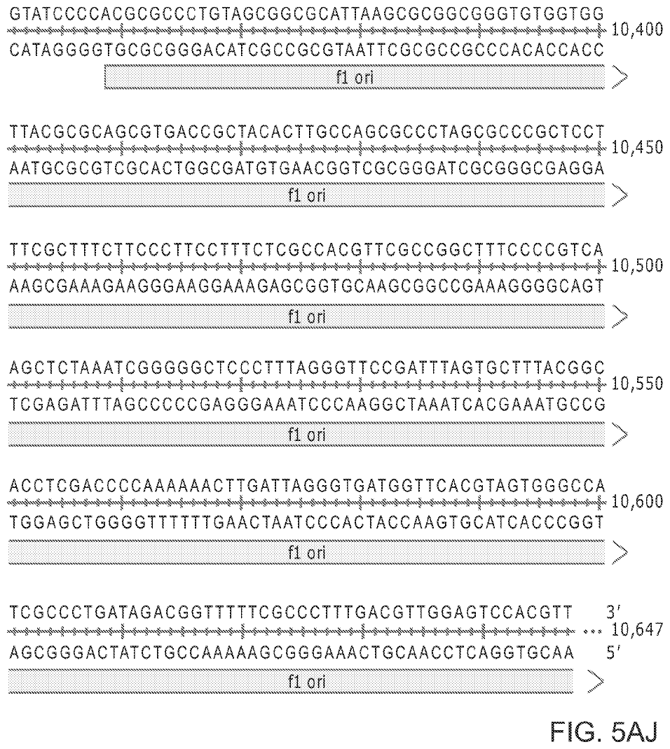

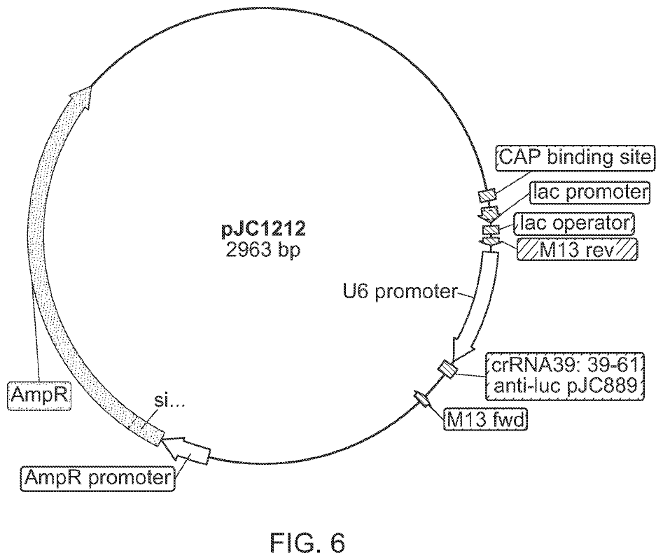

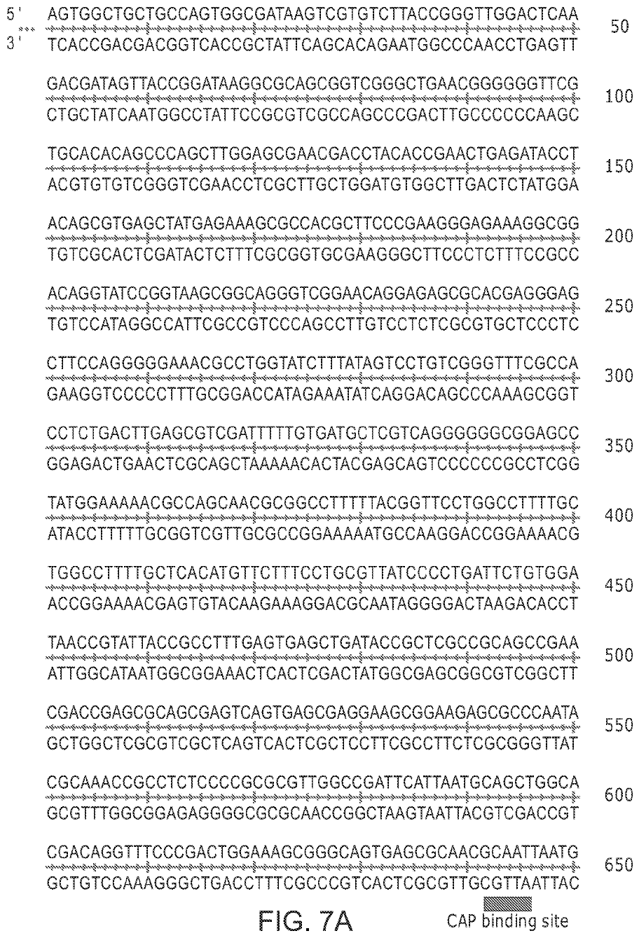

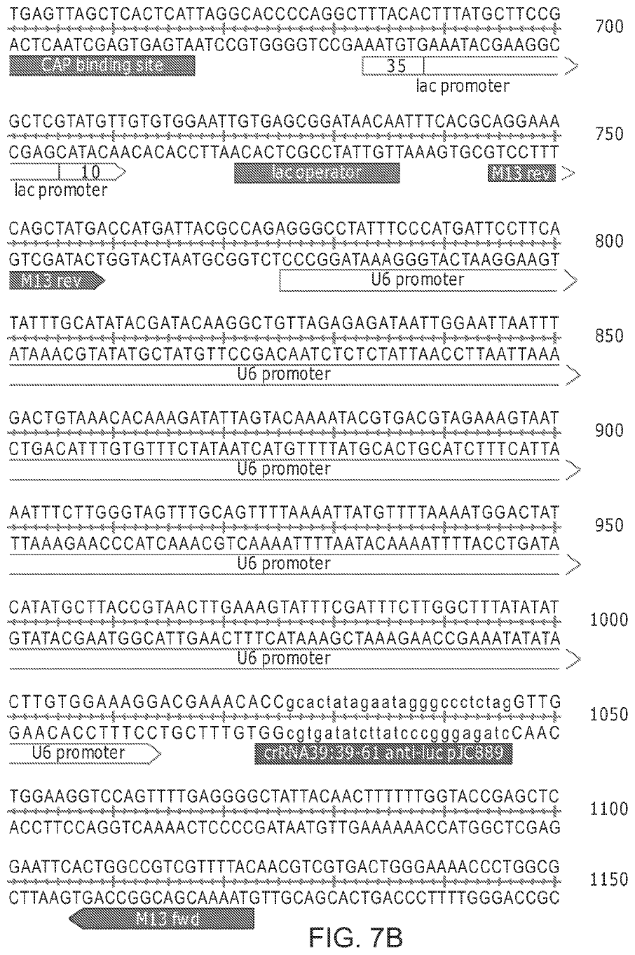

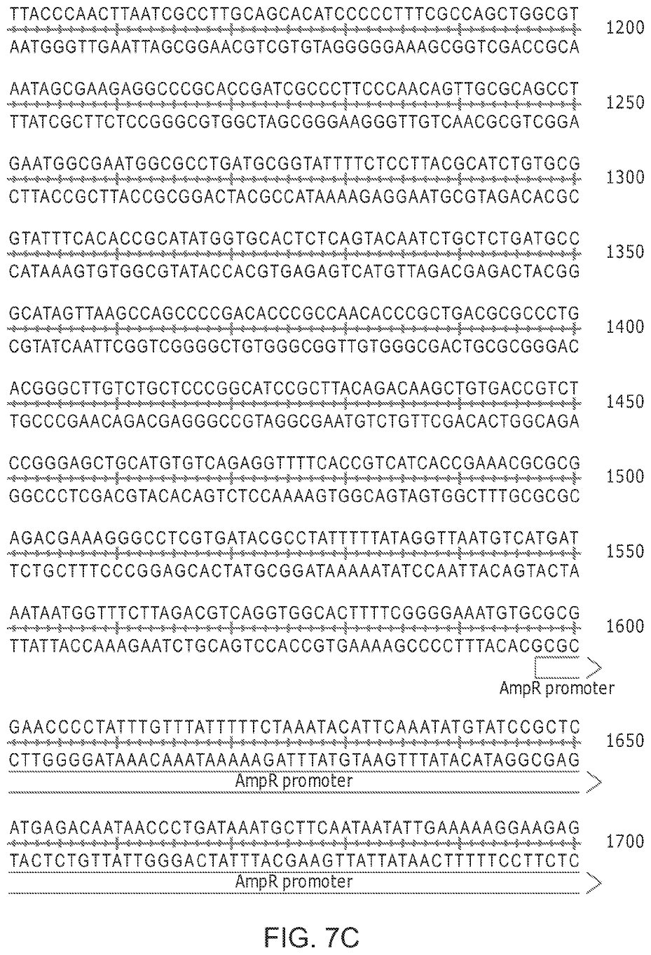

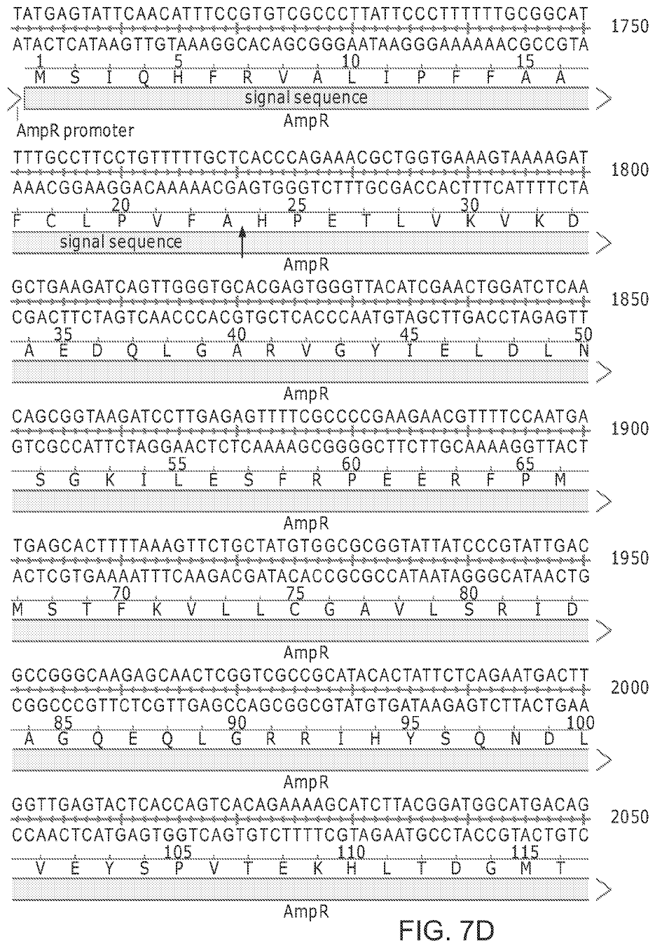

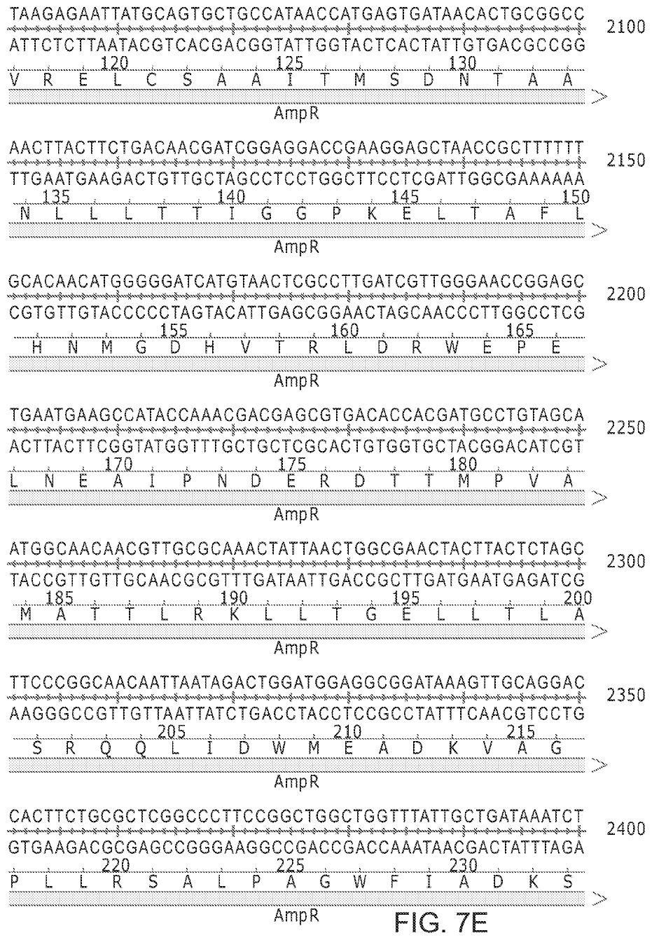

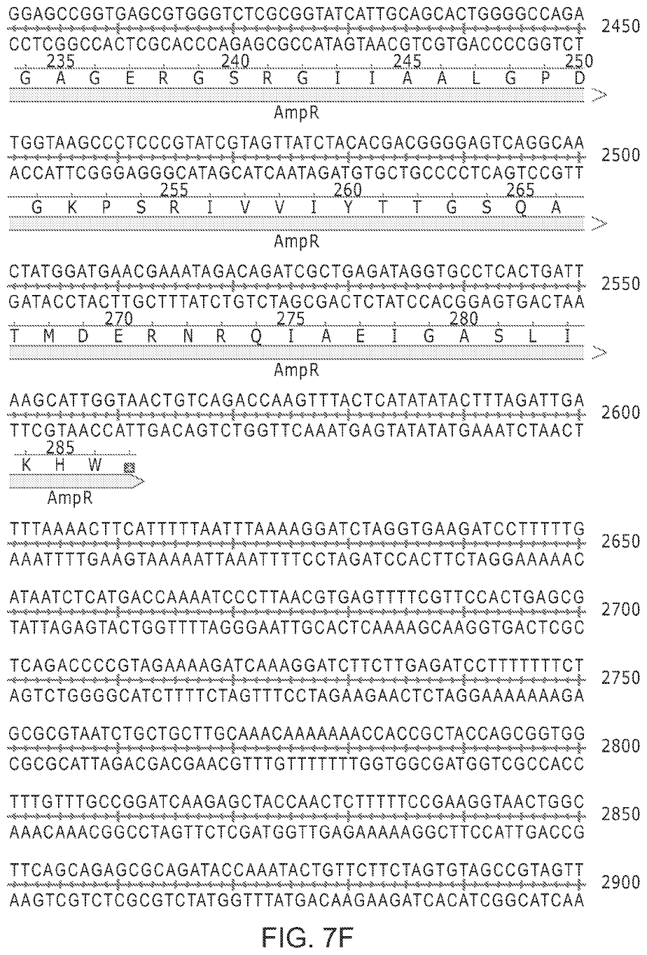

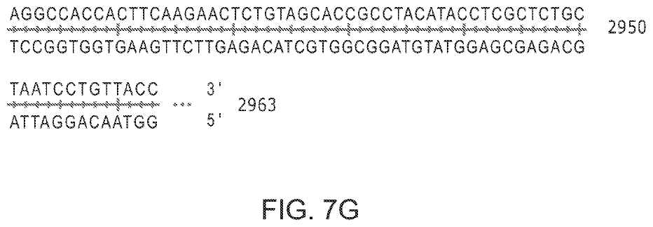

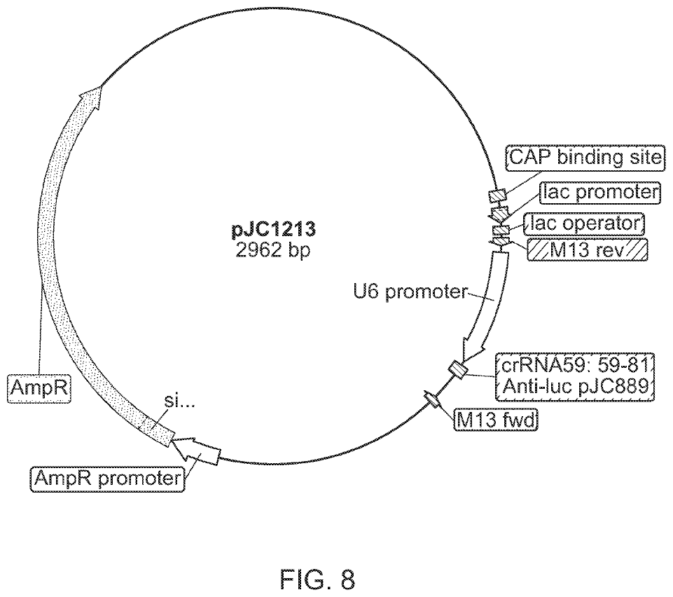

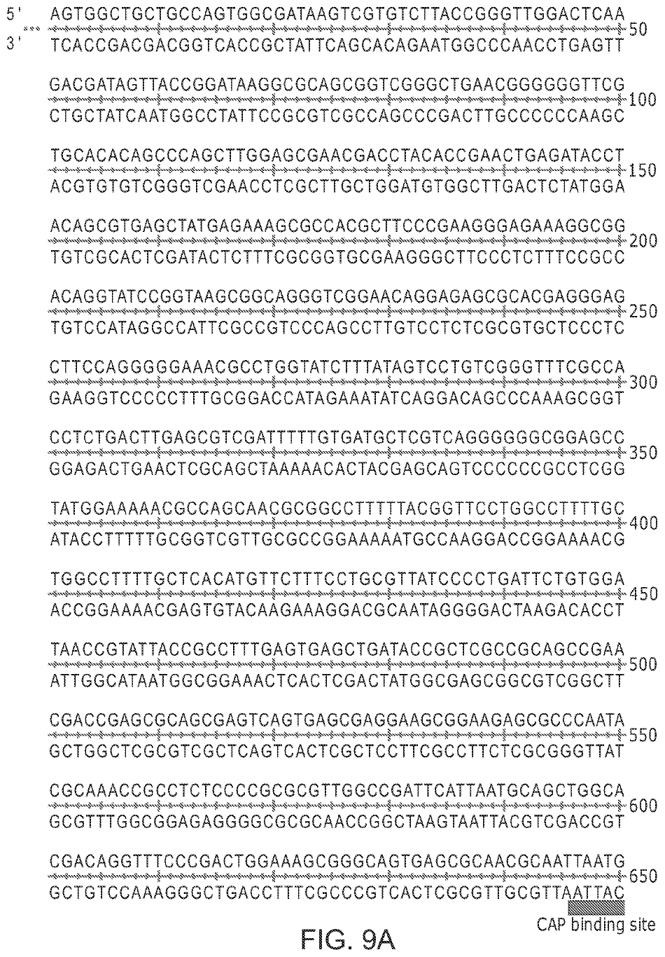

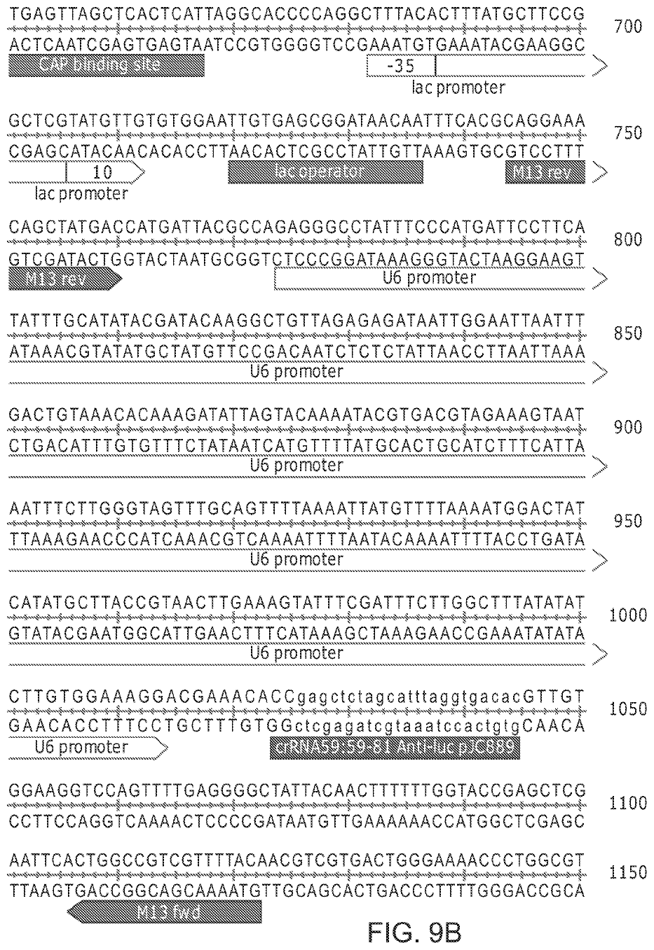

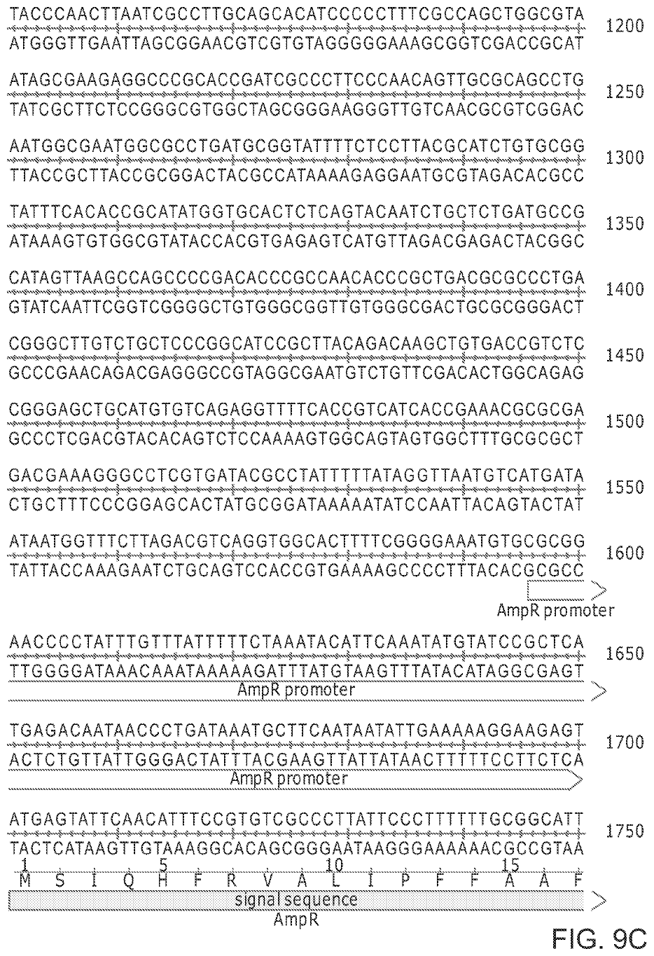

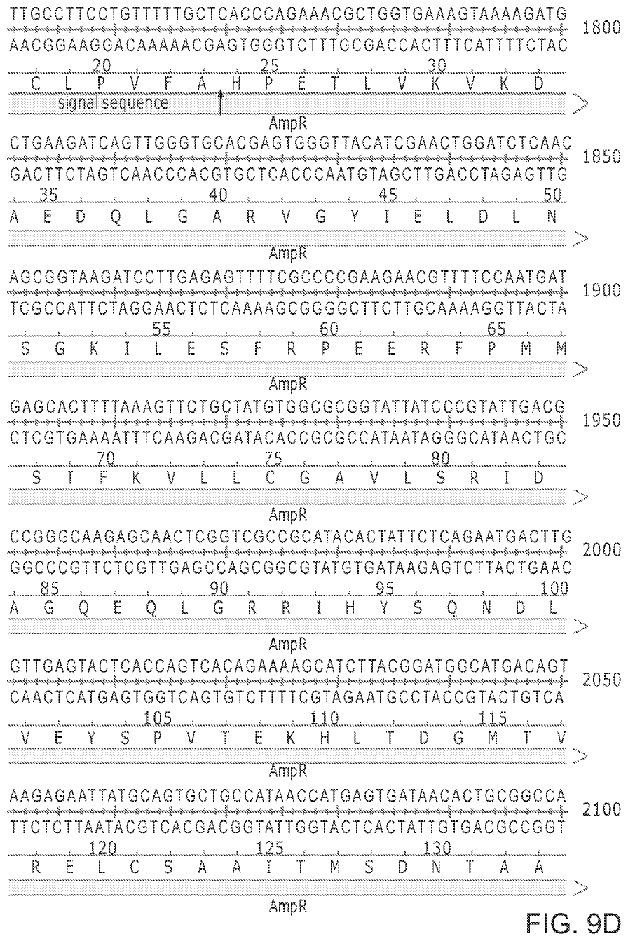

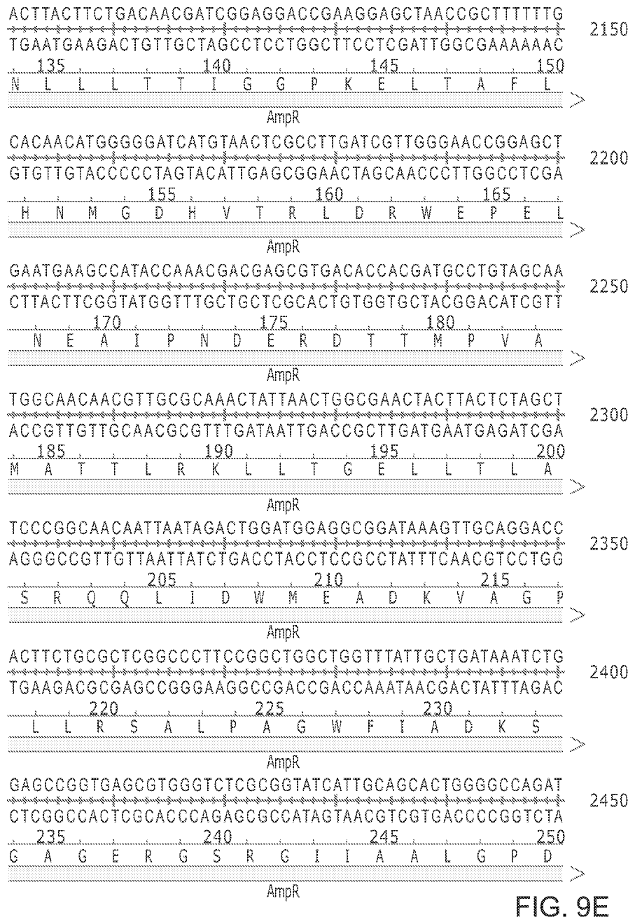

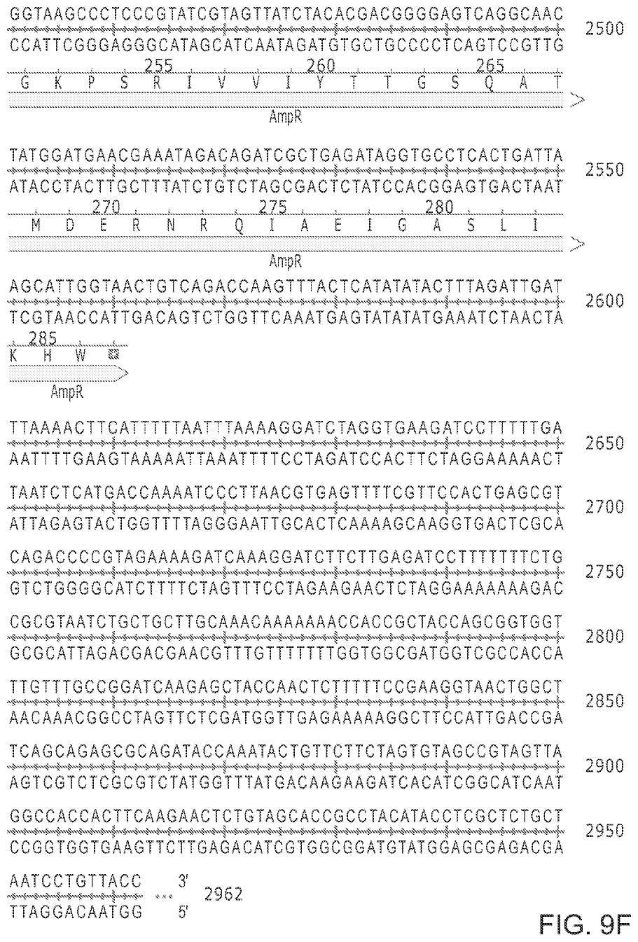

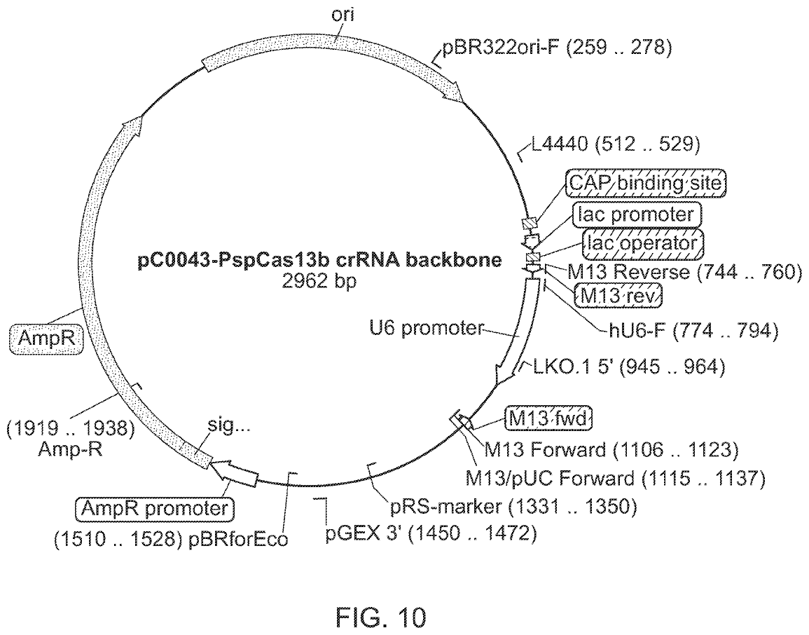

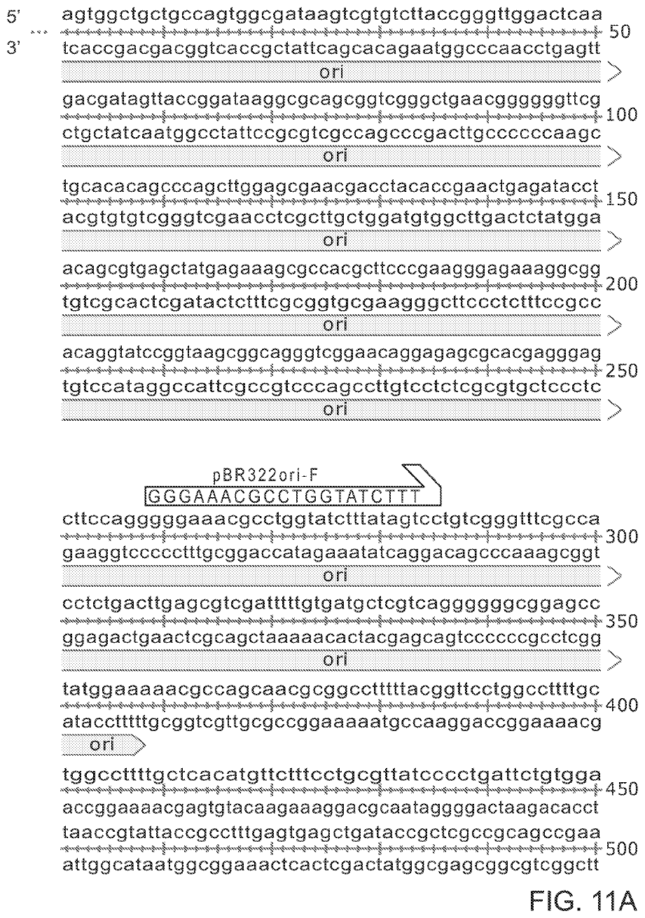

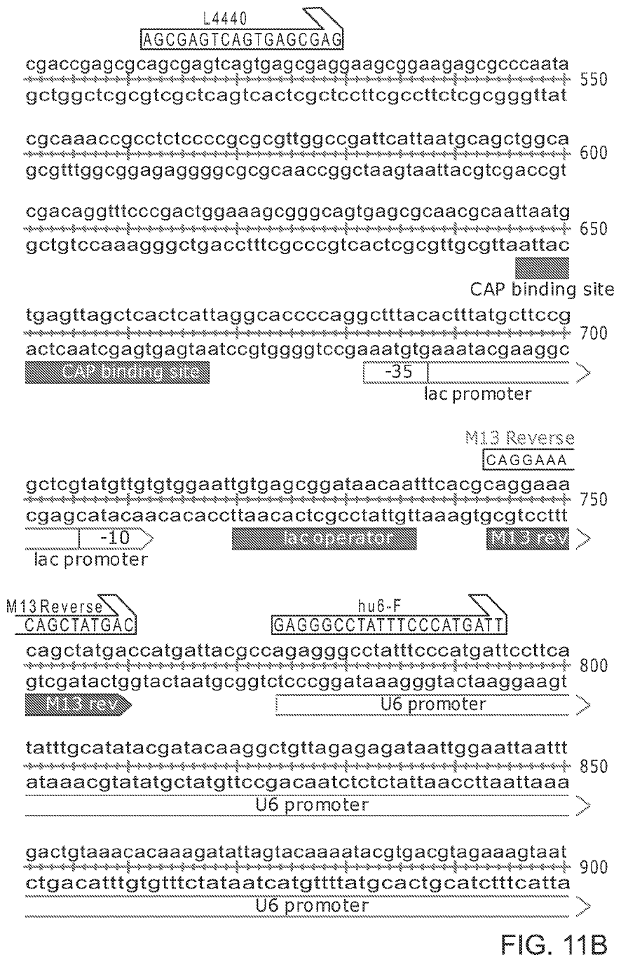

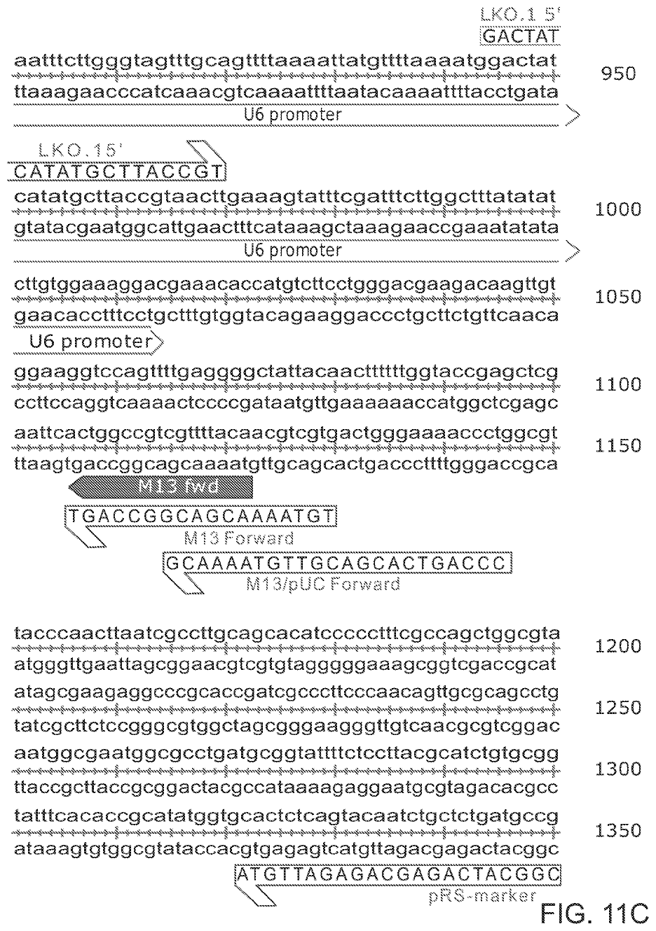

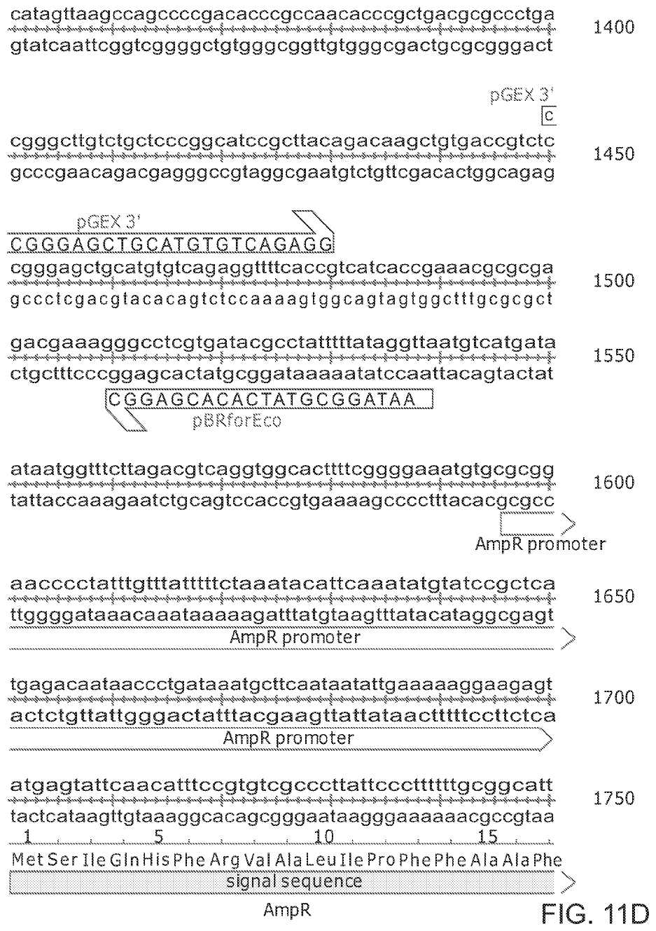

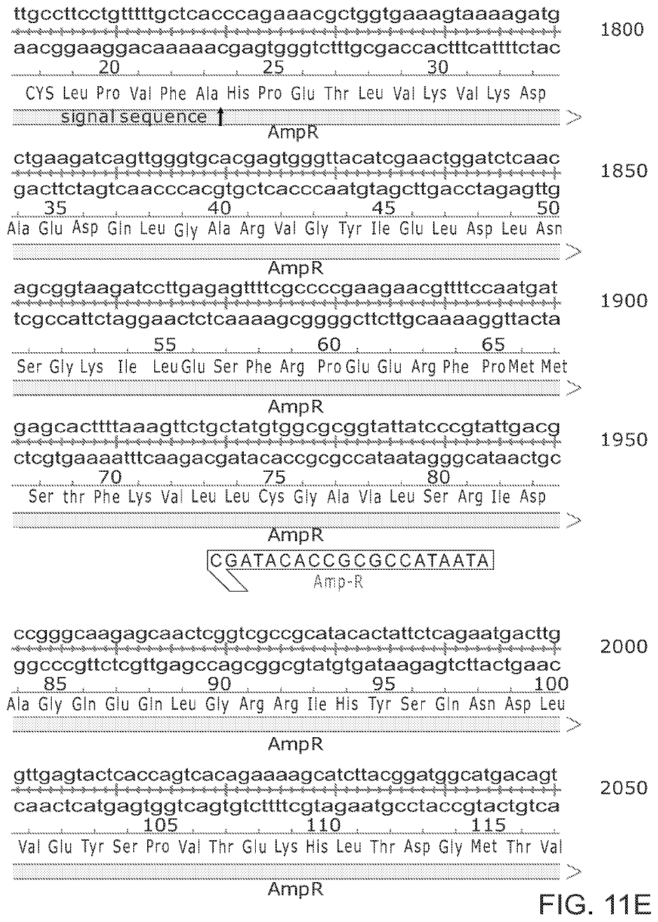

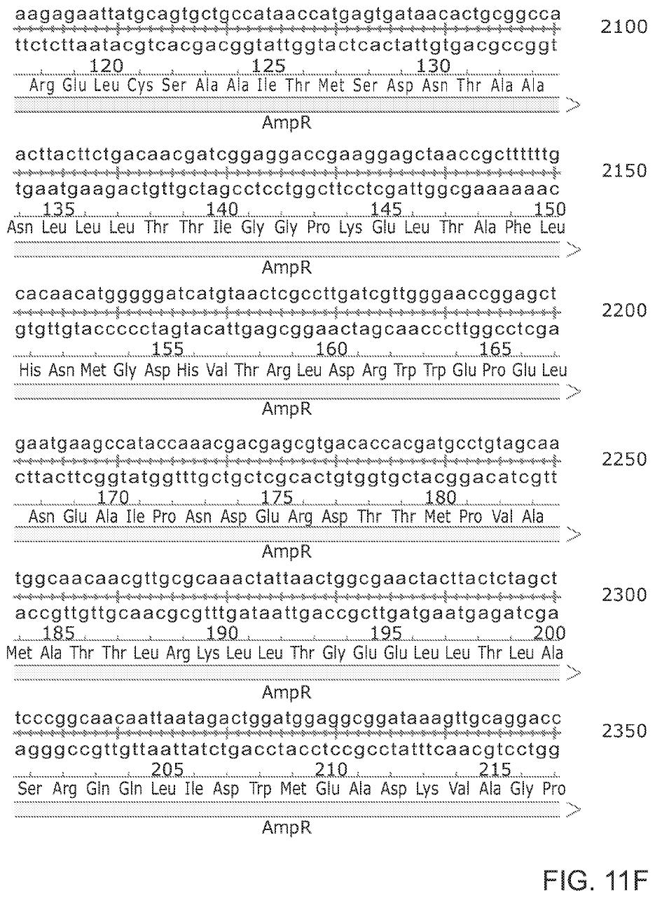

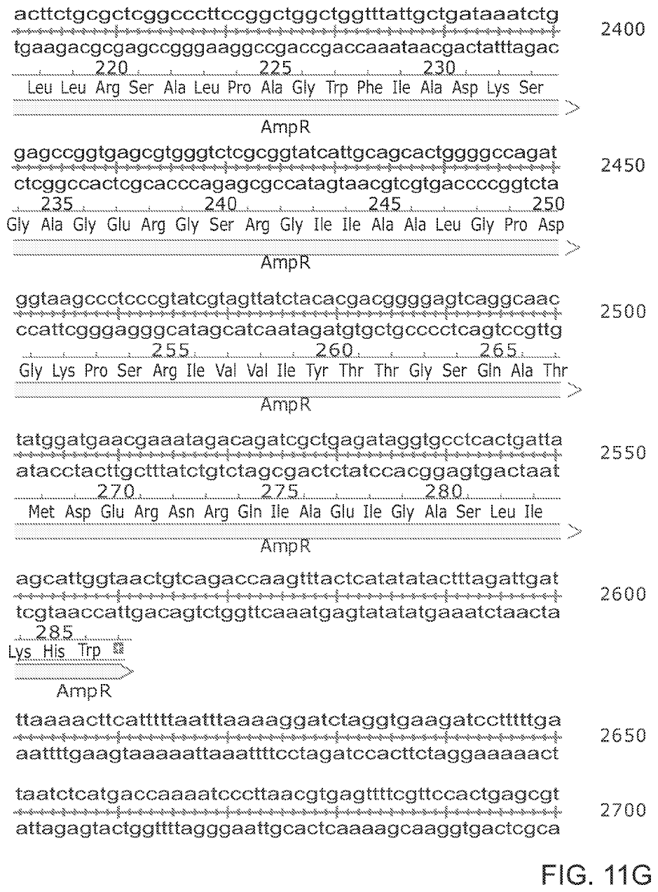

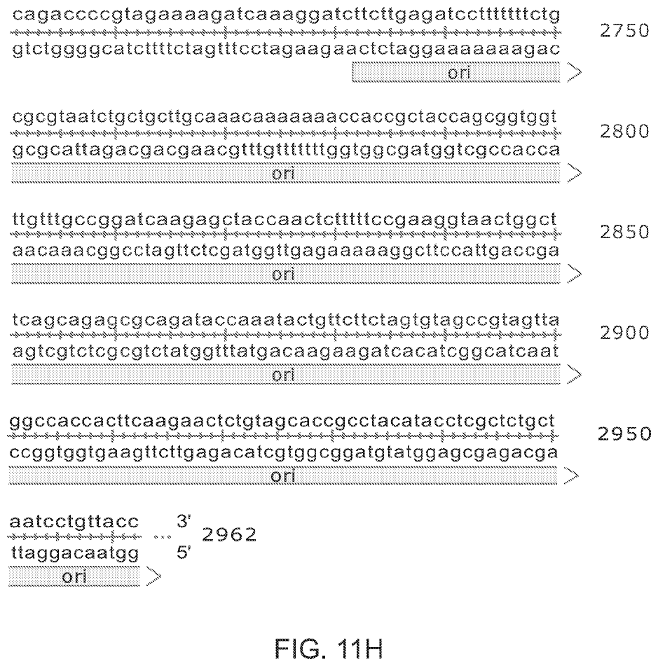

HEK-293 cells were co-transfected with pJC1211 expressing dCas13b plasmid (pJC1211; SEQ ID NO: 50; FIG. 4; FIGS. 5A-5AB) encoding the PABPC1 tethered dCas13b (SEQ ID NO: 49); a plasmid expressing the luc reporter (pJC 889; Addgene plasmid #18964); and one of either pJC1212 (expressing gRNA1 (FIG. 6; FIGS. 7A-7G; SEQ ID NO: 52) pJC1313 (expressing gRNA1 (FIG. 8; FIGS. 9A-9F; SEQ ID NO: 53) or the empty vector Addgene plasmid 103854 (pC0043-PspCas13b crRNA backbone Addgene plasmid #103854; FIG. 10; FIGS. 11A-11H; SEQ ID NO: 54).

Cells were lysed 48 hours after transfection for luciferase detection, and the Promega Luciferase Assay System was applied according to the manufacturer's protocol (Promega E1500). The results show roughly 2-3 fold stimulation over control using two guide RNAs targeting 3′UTR of the luciferase construct (FIG. 3B).

### Example 2: In Vitro and In Vivo Posttranscriptional Regulation of mRNA with PAB1-dCas13b and NAT10-dCas13b Fusion Proteins

Various mRNA regulon therapies will be used as disease modifying therapies in the treatment of monogenic neurological disorders with haploinsufficiency such as Dravet Syndrome and Rett Syndrome. The proteins PABPC1 and NAT10 will each be tethered to an RNA effector protein, e.g., dCas13b and expressed in vitro and in vivo with a gRNA targeting an mRNA of an active allele of a gene associated with Dravet Syndrome (e.g., SCN1A) or a gRNA targeting an mRNA of an active allele of a gene associated with Rett Syndrome (e.g., MECP2).

### Example 3: Enhancing mRNA Expression In Vitro

### Cas13b In Vitro Optimization

A Cas13b/PABPC1 construct was generated by cloning the PCR amplified human PABPC1 in pC0054-CMV-dPspCas13b-longlinker-ADAR2DD (Addgene 103870) (pJC1206) (Table 8). The PC1-MS2V5-PABPC1 plasmid (Addgene #65807) was used as a template for PABPC1 amplification (primers are listed in Table 9). Briefly, pJC1206 was site mutated at nucleotide 5606 to make a unique BamHI site; this construct is hereafter referred to as pJC1210. The ADAR2DD sequence was then removed from using BamHI+NotI and replaced with PCR amplified PABPC1. The generated construct is pJC1211, which includes the full human PABPC1 sequence (Table 8). pJC1246 was created by digesting pJC1211 with BamH1/Not1 and inserting PCR product amplified from oJC5001/oJC5240. pJC1280 was created by cutting pJC1211 with BamH1/Not1 and re-ligating after blunting the ends.

Multiple sgRNAs targeting the 3′UTR of the genes of interest were designed using the ‘nygenome’ online tool for the prediction dCas13b guide (Cas13design (nygenome.org) (Table 7). These guides were individually cloned into PC0043-Cas3b-crRNA backbone (Addgene # #103854) (pJC1208) at BbsI sites. The reaction mix, including forward and reverse oligos in 1×NEB buffer 3.1 was incubated for 5 and 10 minutes at 95 and 100 degrees, then cooled down in the room temperature for 2 hours. Prepared oligos ligated into pJC1208 using T4 DNA ligase (NEB) following the GreenGate protocol.

The RNA amplifier technology was tested in HEK293, HepG2, and SH-SY5Y (ATCC CRL 2266) cell lines. HEK293 and HepG2 cell lines were grown in Dulbecco's Modified Eagle's Medium (DMEM) with 10% FBS, and SH-SY5Y cell lines were grown in Eagle's Minimum Essential Medium (EMEM) with 10% FBS following ATCC guidelines.

Cells were transiently co-transfected with individual sgRNAs along with the dCas13b-PABPC1 fusion plasmid for 48 hours using Opti-MEM Reduced Serum Medium (Gibco) and GeneXPlus Transfection Reagent (ATCC ACS-4004). RNA was isolated using the direct-zol RNA Kit (Zymo Research) following the manufacturer's protocol. cDNA was synthesized using SuperScript III First-Strand Synthesis System (Invitrogen), and qPCR was performed in Applied Biosystems Real-Time PCR instrument using PowerUp™ SYBR Green master mix (Applied Biosystems) and designed primers (Table 9). The data were analyzed using the CT value compared to a no sgRNA transfection and normalized to ACTB as a housekeeping gene.

Protein was isolated using RIPA buffer, and the western blotting was performed using Mini-protean TGX 4-15% gels (BIO-RAD). The following antibodies were used for immunoblotting according to the manufacturer's suggested concentrations; anti-GAPDH (6C5) (Santa Cruz Biotechnology), anti-MeCP2 (D4F3) (Cell Signaling Technology), anti-CHD2 (cat #4170) (Cell Signaling Technology), anti-PTEN (cat #9552) (Cell Signaling Technology), anti-SynGAP (cat #3200) (Cell Signaling Technology), and anti-pan-Shank, clone N23B/49 (Cat #MABN24) (Millipore).

### Luciferase Assay

HEK293 cells were transfected with different ratios of pJC889 (Luciferase-pcDNA3 Addgene #18964), pJc1211, and individually two distinct guide RNAs targeting 3′UTR of firefly luciferase transcripts. The optimal ratio (0.2:1:1.5) of plasmid was chosen for this experiment. For luminescence detection, cells were lysed in 100 μL 1× Passive Lysis Buffer (Promega). The lysate was mixed with ONE-Glo EX Reagent (Promega) following the manufacture protocol, and Luminescence was measured using a Lumat LB9507 Luminometer (Berthold Technologies).

### AlphaFold Method

Structural predictions of fusion proteins and native Cas13b and PABP were generated using AlphaFold v2.0 as pulled from the github repository located at the URL github.com/deepmind/alphafold from commit ‘1d43aaff941c84dc56311076b58795797e49107b’ (ref15). Both native and customized fusion FASTAs were processed according to the AlphaFold documentation using the provided Docker script with the following parameters: ‘—max_template_date=2020 May 14—preset=reduced_dbs’. Relaxed predicted structures with the highest pLDDT scores were used for interpretation of the corresponding input FASTAs.

### Quantifications and Statistical Analysis

All data are shown in figures as bar charts were quantified as mean±standard error. Results were considered significant at p<0.05 as noted throughout figure legends. All experiments, unless otherwise indicated, have been run based on the common practice at least for three biological repeats.

TABLE 8
List of backbone plasmids and generated construct

| Plasmid number | Description |
|---|---|
| pJC1204 | PC1-MS2V5-PABPC1 plasmid Addgene#65807 |
| pJC1206 | pC0054-CMV-dPspCas13b-longlinker-ADAR2DD |
| | Addgene 103870 |
| pJC1208 | PC0043-Cas3b-crRNA backbone Addgene# #103854 |
| pJC1210 | pJC1204 mutated at nucleotide 5606 |
| pJC1211 | pJC1210 digested to remove ADAR2DD and replace |
| | with human PABPC1 |
| pJC1246 | dCas13b-MLLE |
| pJC1280 | dCas13b |
| pJC889 | Luciferase-pcDNA3 Addgene #18964 |

TABLE 9
List of the primers used

| | | | SEQ |
|---|---|---|---|
| | | | ID |
| | Primer | Sequence | NO: |
| oJC4996 | Site mutation | TCAAGCTGCCCAAGGACCGCATCCACAGCGAGAAGTCCAAC | 73 |
| | oligoA | | |
| oJC4996 | Site mutation | GTTGGACTTCTCGCTGTGGATGCGGTCCTTGGGCAGCTTGA | 74 |
| | oligoB | | |
| oJC5001 | PABPC1 F | AATGGGATCCTGGCAAGCCCATC | 75 |
| oJC5001 | PABPC1 R | CTAAAGGGAAGCGGCCGCTTAAA | 76 |
| oJC5065 | MeCP2-R | CAGTTCCTGGAGCTTTGGGAGATT | 77 |
| oJC5065 | MeCP2-F | GTGTATTTGATCAATCCCCAG GGAA | 78 |
| oJC5067 | Actb-F | CACCATTGGCAATGAGCGGTTC | 79 |
| oJC5068 | Actb-R | AGGTCTTTGCGGATGTCCACGT | 80 |
| oJC5240 | MLL-F | GGGATATCGTTCGCAATCCTCAG | 81 |
| oJC5245 | SynGAP-F | TCTAGGCGCTATAGAGCAAACAGG | 82 |
| oJC5246 | SynGAP-R | CAGAGACCCTCAGCTTCCAG | 83 |
| oJC5546 | SHANK3-F | AGGATCACACCCGCCGAGATTA | 84 |
| oJC5547 | SHANK3-R | CTACAGACTTGGTCCGTGGAATC | 85 |
| oJC5548 | PTEN-F | TGAGTTCCCTCAGCCGTTACCT | 86 |
| oJC5549 | PTEN-R | GAGGTTTCCTCTGGTCCTGGTA | 87 |
| oJC5550 | CHD2-F | CGAAAACAGGCACTGGACCACT | 88 |
| OJC5551 | CHD2-R | GATGACGACTGTGTCCGCTGAA | 89 |

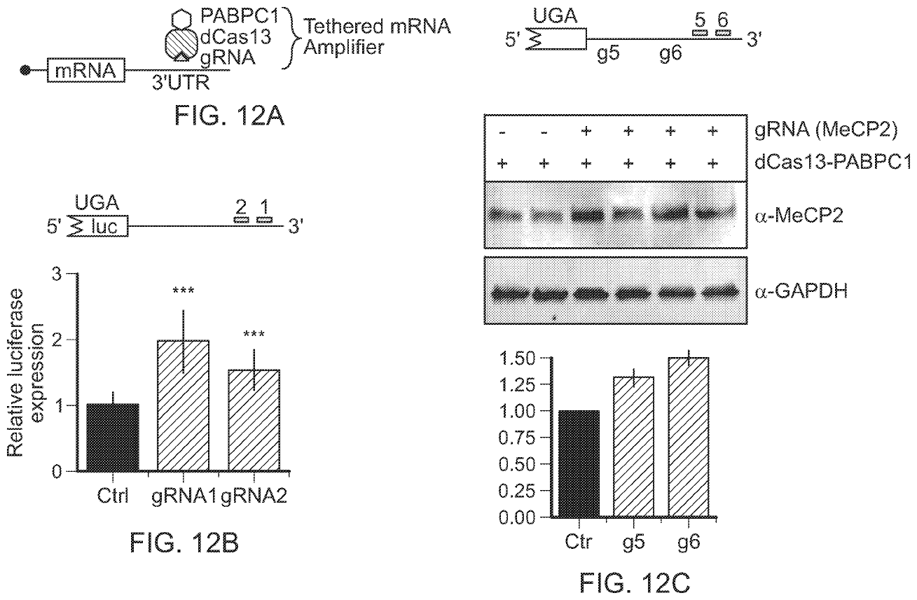

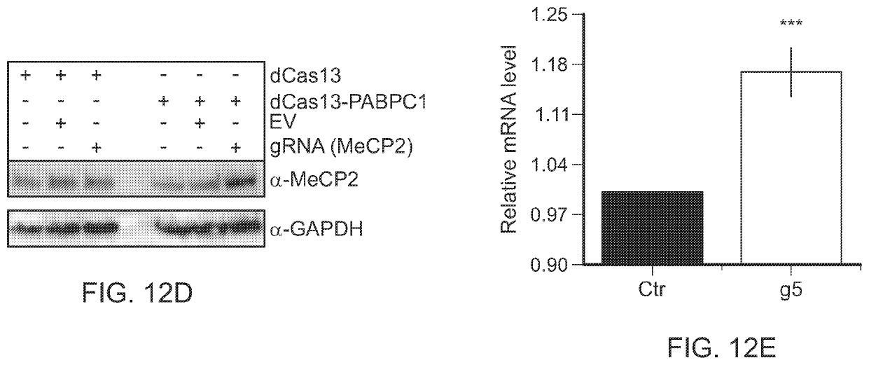

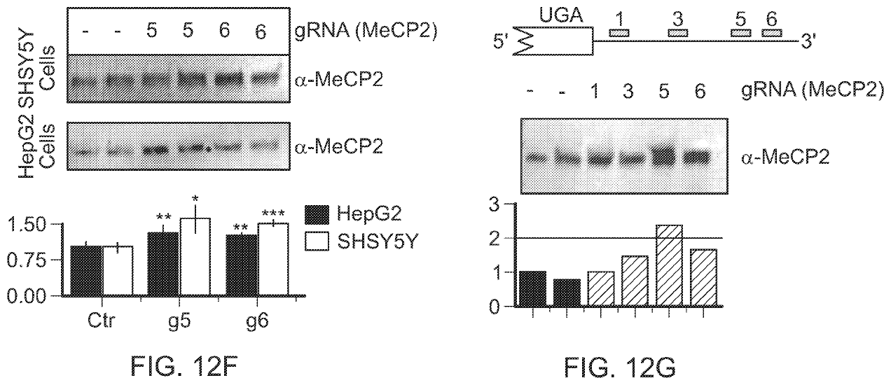

mRNA expression was increased by tethering a known translational stimulator, PABPC1, to the 3′UTR of a target mRNA. Tethering was achieved by fusing PABPC1 to the RNA binding protein dCas13b and co-expressing a guide RNA (gRNA). The gRNA is critical in that it has anti-sense homology to specific mRNAs and a short hairpin required for dCas13b binding (FIG. 12A). It was demonstrated that this gRNA-targeted tethering enhances both reporter and endogenous mRNAs in a gRNA-dependent manner. First, using HEK293 cells the Tethered mRNA Amplifier was co-transfected alongside a luciferase reporter construct. An approximate 1.5 to 2-fold increase in reporter protein amount was seen when gRNAs directed against the 3′UTR of the luciferase reporter are present. No stimulation occurred when the dCas13b-PABPC1 fusion is expressed alone (FIG. 12B; Ctrl). Continuing in HEK293 cells, the translation of an endogenous mRNA, MeCP2 was stimulated. An approximate 1.5-fold stimulation of translation was seen using two distinct gRNAs directed against the endogenous MeCP2 transcript's 3′UTR (FIG. 12C). A mild increase of 15% in MeCP2 transcript steady-state levels was observed when in the presence of the mRNA Amplifier (FIG. 12D). These data suggest that the stimulatory role of the Tethered mRNA Amplifier is through both mRNA stability and mRNA translation-known roles for PABPC1 in regulating mRNA metabolism.

### Example 4: Enhancing mRNA Expression in Multiple Cell Types

It was further shown that the Tethered mRNA Amplifier enhances mRNA expression in multiple cell types; a stimulatory effect on MeCP2 protein expression is seen in SH-SY5Y (a neuronal cell line) and HepG2 (a liver cell line; FIG. 12E). Finally, the effect of the Tethered mRNA amplifier was shown to be tunable by moving the gRNA to distinct positions within the 3′UTR. In the case of MeCP2 the strongest stimulatory effect was observed as the gRNA was moved closer to the 3′ end of the transcript (FIG. 12F).

### Example 5: Enhancing mRNA Expression on Different mRNA Transcripts

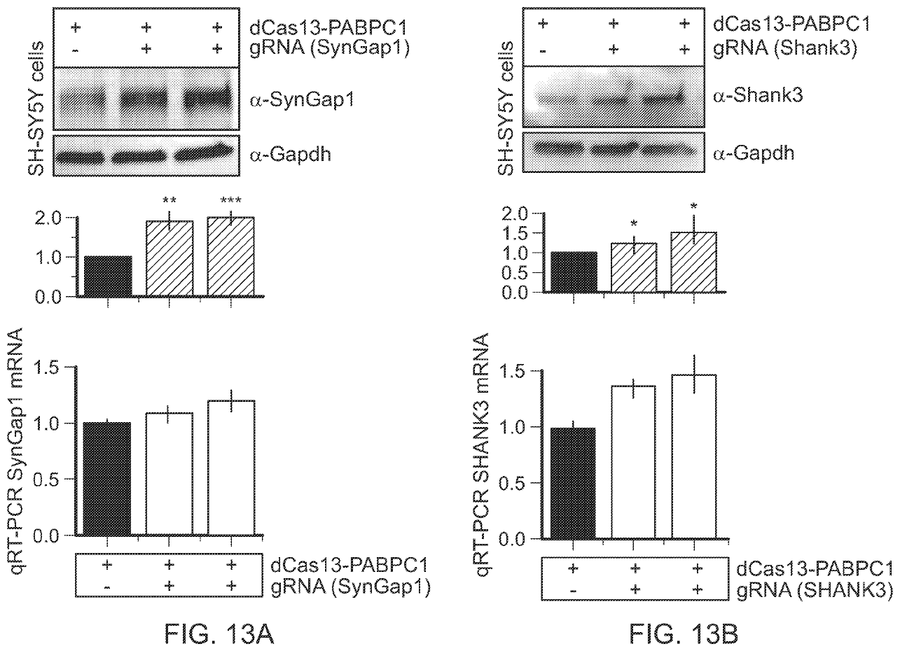

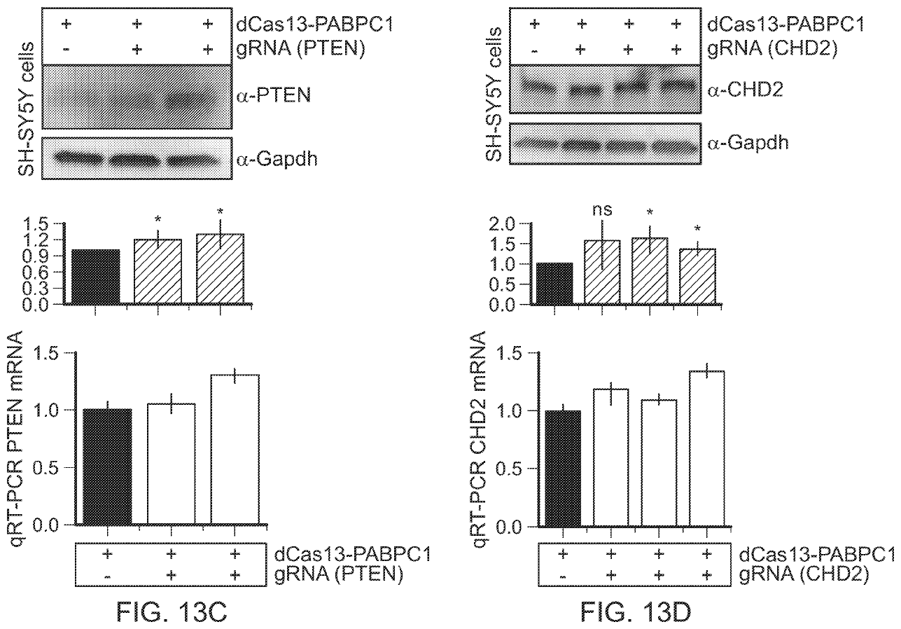

Next, the Tethered mRNA Amplifier was tested on other transcripts associated with haploinsufficiency disorders. Using SH-SY5Y cells, a model for neurodegenerative disorders, a gRNA-dependent translational stimulation of SYNGAP115, SHANK316, CHD217, and PTEN18 mRNAs was observed (FIG. 13A-13D). The loss of function of one allele for each of these genes is associated with autism spectrum disorders. In all cases, the stimulatory effect seen was between 1.2 to 2.0-fold for protein expression with an approximately 15-20% increase in mRNA levels. These data demonstrate that the Tethered mRNA Amplifier can be used as a gene therapy candidate for haploinsufficiency and is portable across multiple transcripts of clinical relevance.

### Example 6: Minimizing the Size of the Tethered mRNA Amplifier Fusion Protein

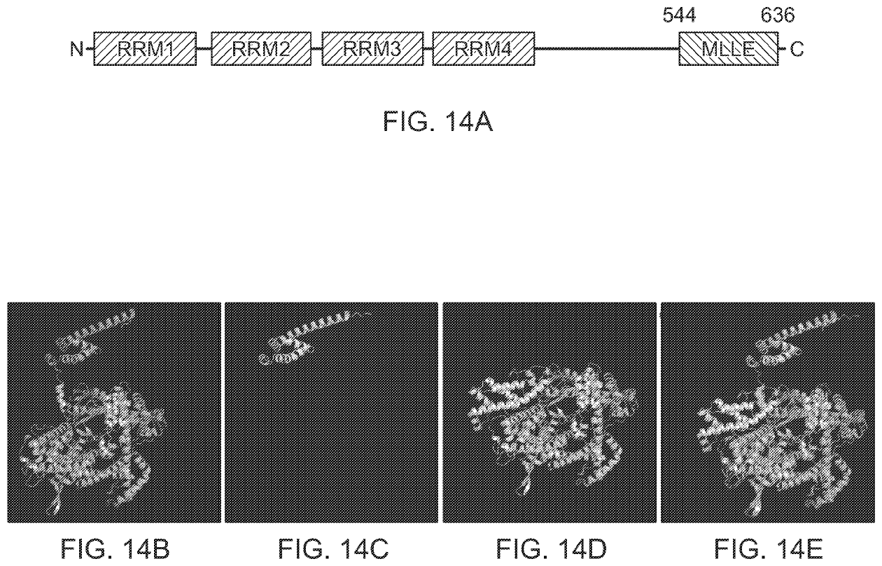

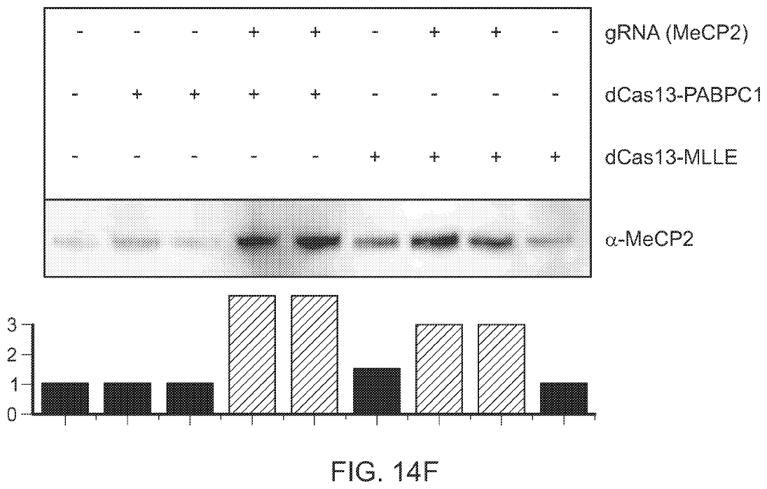

Finally, it is known that gene therapy vectors such as AAV have payload size limitations of approximately 4.5 kb. To minimize the Tethered mRNA Amplifier (5.2 kb), specific truncations of PABPC1 were made and their efficacy on MeCP2 expression was tested. PABPC1 contains four RNA-recognition motifs (RRM1-4) at its N-terminus followed by a linker and a Mademoiselle (MLLE) domain at the C-terminus (FIG. 14A). The RRM domains bind to poly(A) tails while the MLLE domain is known to regulate its stimulatory role in translation. Since PABPC1 was artificially and specifically tethered to mRNAs independent of PABPC1 poly(A)-binding capacity, it was reasoned the RRMs would be dispensable for the Tethered mRNA Amplifier's function. Therefore, just the MLLE domain was fused to dCas13b (3.2 KB). As a first test, the putative folding pattern of this new fusion was analyzed with an in silico approach using AlphaFold v2.0. As seen in FIGS. 14B-14E, removal of the RRM domains had minimal impact on the folding of either the MLLE domain or dCas13b itself. In HEK293 cells it was observed that this minimal construct also stimulated MeCP2 mRNA expression in a gRNA-dependent fashion to a similar extent as the full-length construct (FIG. 14F). Thus, the Tethered mRNA Amplifier approach was shown to be adaptable for use in current clinically efficacious gene therapy vectors.

### REFERENCES

1. Escayg, A., et al., Mutations of SCN1A, encoding a neuronal sodium channel, in two families with GEFS+2. Nature genetics, 2000. 24 (4): p. 343-345.
2. Claes, L., et al., De novo mutations in the sodium-channel gene SCN1A cause severe myoclonic epilepsy of infancy. The American Journal of Human Genetics, 2001. 68 (6): p. 1327-1332.
3. Dravet, C., The core Dravet syndrome phenotype. Epilepsia, 2011. 52 (s2): p. 3-9.
4. Genton, P., R. Velizarova, and C. Dravet, Dravet syndrome: The long-term outcome. Epilepsia, 2011. 52 (s2): p. 44-49.
5. Dravet, C. and H. Oguni, Chapter 65—Dravet syndrome (severe myoclonic epilepsy in infancy), in Handbook of Clinical Neurology, O. Dulac, M. Lassonde, and H. B. Sarnat, Editors. 2013, Elsevier. p. 627-633.
6. Cooper, M. S., et al., Mortality in Dravet syndrome. Epilepsy Research, 2016. 128: p. 43-47.
7. Higurashi, N., V. Broccoli, and S. Hirose, Genetics and gene therapy in Dravet syndrome. Epilepsy & Behavior, 2021: p. 108043.
8. Lueck, J. D., et al., Engineered transfer RNAs for suppression of premature termination codons. Nature Communications, 2019. 10 (1): p. 822.
9. Howard, M., R. A. Frizzell, and D. M. Bedwell, Aminoglycoside antibiotics restore CFTR function by overcoming premature stop mutations. Nat Med, 1996. 2 (4): p. 467-9.
10. Arakawa, M., et al., Negamycin restores dystrophin expression in skeletal and cardiac muscles of mdx mice. J Biochem, 2003. 134 (5): p. 751-8.
11. Welch, E. M., et al., PTC124 targets genetic disorders caused by nonsense mutations. Nature, 2007. 447 (7140): p. 87-91.
12. Roy, B., et al., Ataluren stimulates ribosomal selection of near-cognate tRNAs to promote nonsense suppression. Proc Natl Acad Sci USA, 2016. 113 (44): p. 12508-12513.
13. Berecki, G., et al., SCN1A gain of function in early infantile encephalopathy. Ann Neurol, 2019. 85 (4): p. 514-525.
14. Dhifallah, S., et al., Gain of Function for the SCN1A/hNav1.1-L1670W Mutation Responsible for Familial Hemiplegic Migraine. Front Mol Neurosci, 2018. 11: p. 232.
15. Kotecha, B. and G. P. Richardson, Ototoxicity in vitro: effects of neomycin, gentamicin, dihydrostreptomycin, amikacin, spectinomycin, neamine, spermine and poly-L-lysine. Hear Res, 1994. 73 (2): p. 173-84.
16. Dai, W. J., et al., CRISPR-Cas9 for in vivo Gene Therapy: Promise and Hurdles. Mol Ther Nucleic Acids, 2016. 5: p. e349.
17. Peng, R., G. Lin, and J. Li, Potential pitfalls of CRISPR/Cas9-mediated genome editing. FEBS J, 2016. 283 (7): p. 1218-31.
18. Coller, J. and M. Wickens, Chapter Fourteen—Tethered Function Assays: An Adaptable Approach to Study RNA Regulatory Proteins, in Methods in Enzymology, J. Lorsch, Editor. 2007, Academic Press. p. 299-321.
19. Coller, J. M., N. K. Gray, and M. P. Wickens, mRNA stabilization by poly (A) binding protein is independent of poly (A) and requires translation. Genes & development, 1998. 12 (20): p. 3226-3235.
20. Cox, D. B., et al., RNA editing with CRISPR-Cas13. Science, 2017. 358 (6366): p. 1019-1027.
21. Gootenberg, J. S., et al., Nucleic acid detection with CRISPR-Cas13a/C2c2. Science, 2017. 356 (6336): p. 438-442.
22. Terns, M. P., CRISPR-Based Technologies: Impact of RNA-Targeting Systems. Molecular Cell, 2018. 72 (3): p. 404-412.
23. Nietfeld, W., H. Mentzel, and T. Pieler, The Xenopus laevis poly (A) binding protein is composed of multiple functionally independent RNA binding domains. The EMBO Journal, 1990. 9 (11): p. 3699-3705.
24. Deardorff, J. A. and A. B. Sachs, Differential effects of aromatic and charged residue substitutions in the RNA binding domains of the yeast Poly (A)-binding protein11Edited by K. Nagai. Journal of Molecular Biology, 1997. 269 (1): p. 67-81.
25. Arango, D., et al., Acetylation of Cytidine in mRNA Promotes Translation Efficiency. Cell, 2018. 175 (7): p. 1872-1886.e24.
26. Yang, N., et al., Generation of pure GABAergic neurons by transcription factor programming. Nat Methods, 2017. 14 (6): p. 621-628.

SEQUENCES

SEQ ID NO: 1 >NR_148667.2 SCN1A [organism = Homo sapiens] [GeneID-6323]
[transcript = 16]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGAACAGCATAAGAATTATTTCTGAGTGGAGGTGAGGCTTGTCCAAATGTCTTTGCTATCATGG
ATTTCCTGACTCCTACCTGTTTGAGGAATTTCATATGCAGAATAAATGGTAATTAAAATGTGCAGGATGACAAGATGG
AGCAAACAGTGCTTGTACCACCAGGACCTGACAGCTTCAACTTCTTCACCAGAGAATCTCTTGCGGCTATTGAAAGAC
GCATTGCAGAAGAAAAGGCAAAGAATCCCAAACCAGACAAAAAAGATGACGACGAAAATGGCCCAAAGCCAAATAGTG
ACTTGGAAGCTGGAAAGAACCTTCCATTTATTTATGGAGACATTCCTCCAGAGATGGTGTCAGAGCCCCTGGAGGACC
TGGACCCCTACTATATCAATAAGAAAACTTTTATAGTATTGAATAAAGGGAAGGCCATCTTCCGGTTCAGTGCCACCT
CTGCCCTGTACATTTTAACTCCCTTCAATCCTCTTAGGAAAATAGCTATTAAGATTTTGGTACATTCATTATTCAGCA
TGCTAATTATGTGCACTATTTTGACAAACTGTGTGTTTATGACAATGAGTAACCCTCCTGATTGGACAAAGAATGTAG
AATACACCTTCACAGGAATATATACTTTTGAATCACTTATAAAAATTATTGCAAGGGGATTCTGTTTAGAAGATTTTA
CTTTCCTTCGGGATCCATGGAACTGGCTCGATTTCACTGTCATTACATTTGCGTACGTCACAGAGTTTGTGGACCTGG
GCAATGTCTCGGCATTGAGAACATTCAGAGTTCTCCGAGCATTGAAGACGATTTCAGTCATTCCAGGCCTGAAAACCA
TTGTGGGAGCCCTGATCCAGTCTGTGAAGAAGCTCTCAGATGTAATGATCCTGACTGTGTTCTGTCTGAGCGTATTTG
CTCTAATTGGGCTGCAGCTGTTCATGGGCAACCTGAGGAATAAATGTATACAATGGCCTCCCACCAATGCTTCCTTGG
AGGAACATAGTATAGAAAAGAATATAACTGTGAATTATAATGGTACACTTATAAATGAAACTGTCTTTGAGTTTGACT
GGAAGTCATATATTCAAGATTCAAGATATCATTATTTCCTGGAGGGTTTTTTAGATGCACTACTATGTGGAAATAGCT
CTGATGCAGGCCAATGTCCAGAGGGATATATGTGTGTGAAAGCTGGTAGAAATCCCAATTATGGCTACACAAGCTTTG
ATACCTTCAGTTGGGCTTTTTTGTCCTTGTTTCGACTAATGACTCAGGACTTCTGGGAAAATCTTTATCAACTGACAT
TACGTGCTGCTGGGAAAACGTACATGATATTTTTTGTATTGGTCATTTTCTTGGGCTCATTCTACCTAATAAATTTGA
TCCTGGCTGTGGTGGCCATGGCCTACGAGGAACAGAATCAGGCCACCTTGGAAGAAGCAGAACAGAAAGAGGCCGAAT
TTCAGCAGATGATTGAACAGCTTAAAAAGCAACAGGAGGCAGCTCAGCAGGCAGCAACGGCAACTGCCTCAGAACATT
CCAGAGAGCCCAGTGCAGCAGGCAGGCTCTCAGACAGCTCATCTGAAGCCTCTAAGTTGAGTTCCAAGAGTGCTAAGG
AAAGAAGAAATCGGAGGAAGAAAAGAAAACAGAAAGAGCAGTCTGGTGGGGAAGAGAAAGATGAGGATGAATTCCAAA
AATCTGAATCTGAGGACAGCATCAGGAGGAAAGGTTTTCGCTTCTCCATTGAAGGGAACCGATTGACATATGAAAAGA
GGTACTCCTCCCCACACCAGTCTTTGTTGAGCATCCGTGGCTCCCTATTTTCACCAAGGCGAAATAGCAGAACAAGCC
TTTTCAGCTTTAGAGGGCGAGCAAAGGATGTGGGATCTGAGAACGACTTCGCAGATGATGAGCACAGCACCTTTGAGG
ATAACGAGAGCCGTAGAGATTCCTTGTTTGTGCCCCGACGACACGGAGAGAGACGCAACAGCAACCTGAGTCAGACCA
GTAGGTCATCCCGGATGCTGGCAGTGTTTCCAGCGAATGGGAAGATGCACAGCACTGTGGATTGCAATGGTGTGGTTT
CCTTGGTTGGTGGACCTTCAGTTCCTACATCGCCTGTTGGACAGCTTCTGCCAGAGGGAACAACCACTGAAACTGAAA
TGAGAAAGAGAAGGTCAAGTTCTTTCCACGTTTCCATGGACTTTCTAGAAGATCCTTCCCAAAGGCAACGAGCAATGA
GTATAGCCAGCATTCTAACAAATACAGTAGAAGAACTTGAAGAATCCAGGCAGAAATGCCCACCCTGTTGGTATAAAT
TTTCCAACATATTCTTAATCTGGGACTGTTCTCCATATTGGTTAAAAGTGAAACATGTTGTCAACCTGGTTGTGATGG
ACCCATTTGTTGACCTGGCCATCACCATCTGTATTGTCTTAAATACTCTTTTCATGGCCATGGAGCACTATCCAATGA
CGGACCATTTCAATAATGTGCTTACAGTAGGAAACTTGGTTTTCACTGGGATCTTTACAGCAGAAATGTTTCTGAAAA
TTATTGCCATGGATCCTTACTATTATTTCCAAGAAGGCTGGAATATCTTTGACGGTTTTATTGTGACGCTTAGCCTGG
TAGAACTTGGACTCGCCAATGTGGAAGGATTATCTGTTCTCCGTTCATTTCGATTGCTGCGAGTTTTCAAGTTGGCAA
AATCTTGGCCAACGTTAAATATGCTAATAAAGATCATCGGCAATTCCGTGGGGGCTCTGGGAAATTTAACCCTCGTCT
TGGCCATCATCGTCTTCATTTTTGCCGTGGTCGGCATGCAGCTCTTTGGTAAAAGCTACAAAGATTGTGTCTGCAAGA
TCGCCAGTGATTGTCAACTCCCACGCTGGCACATGAATGACTTCTTCCACTCCTTCCTGATTGTGTTCCGCGTGCTGT
GTGGGGAGTGGATAGAGACCATGTGGGACTGTATGGAGGTTGCTGGTCAAGCCATGTGCCTTACTGTCTTCATGATGG
TCATGGTGATTGGAAACCTAGTGGTCCTGAATCTCTTTCTGGCCTTGCTTCTGAGCTCATTTAGTGCAGACAACCTTG
CAGCCACTGATGATGATAATGAAATGAATAATCTCCAAATTGCTGTGGATAGGATGCACAAAGGAGTAGCTTATGTGA
AAAGAAAAATATATGAATTTATTCAACAGTCCTTCATTAGGAAACAAAAGATTTTAGATGAAATTAAACCACTTGATG
ATCTAAACAACAAGAAAGACAGTTGTATGTCCAATCATACAGCAGAAATTGGGAAAGATCTTGACTATCTTAAAGATG
TAAATGGAACTACAAGTGGTATAGGAACTGGCAGCAGTGTTGAAAAATACATTATTGATGAAAGTGATTACATGTCAT
TCATAAACAACCCCAGTCTTACTGTGACTGTACCAATTGCTGTAGGAGAATCTGACTTTGAAAATTTAAACACGGAAG
ACTTTAGTAGTGAATCGGATCTGGAAGAAAGCAAAGAGAAACTGAATGAAAGCAGTAGCTCATCAGAAGGTAGCACTG
TGGACATCGGCGCACCTGTAGAAGAACAGCCCGTAGTGGAACCTGAAGAAACTCTTGAACCAGAAGCTTGTTTCACTG
AAGGCTGTGTACAAAGATTCAAGTGTTGTCAAATCAATGTGGAAGAAGGCAGAGGAAAACAATGGTGGAACCTGAGAA
GGACGTGTTTCCGAATAGTTGAACATAACTGGTTTGAGACCTTCATTGTTTTCATGATTCTCCTTAGTAGTGGTGCTC
TGGCATTTGAAGATATATATATTGATCAGCGAAAGACGATTAAGACGATGTTGGAATATGCTGACAAGGTTTTCACTT
ACATTTTCATTCTGGAAATGCTTCTAAAATGGGTGGCATATGGCTATCAAACATATTTCACCAATGCCTGGTGTTGGC
TGGACTTCTTAATTGTTGATGTTTCATTGGTCAGTTTAACAGCAAATGCCTTGGGTTACTCAGAACTTGGAGCCATCA
AATCTCTCAGGACACTAAGAGCTCTGAGACCTCTAAGAGCCTTATCTCGATTTGAAGGGATGAGGGATAATCTTGCTC
CAACTTGGATGGGGTGGAGCGCTGGTTCCTCCCCTGAGCCCTTTATTATGGGTGGTTGTGAATGCCCTTTTAGGAGCA
ATTCCATCCATCATGAATGTGCTTCTGGTTTGTCTTATATTCTGGCTAATTTTCAGCATCATGGGCGTAAATTTGTTT
GCTGGCAAATTCTACCACTGTATTAACACCACAACTGGTGACAGGTTTGACATCGAAGACGTGAATAATCATACTGAT
TGCCTAAAACTAATAGAAAGAAATGAGACTGCTCGATGGAAAAATGTGAAAGTAAACTTTGATAATGTAGGATTTGGG
TATCTCTCTTTGCTTCAAGTTGCCACATTCAAAGGATGGATGGATATAATGTATGCAGCAGTTGATTCCAGAAATGTG
GAACTCCAGCCTAAGTATGAAGAAAGTCTGTACATGTATCTTTACTTTGTTATTTTCATCATCTTTGGGTCCTTCTTC
ACCTTGAACCTGTTTATTGGTGTCATCATAGATAATTTCAACCAGCAGAAAAAGAAGTTTGGAGGTCAAGACATCTTT
ATGACAGAAGAACAGAAGAAATACTATAATGCAATGAAAAAATTAGGATCGAAAAAACCGCAAAAGCCTATACCTCGA
CCAGGAAACAAATTTCAAGGAATGGTCTTTGACTTCGTAACCAGACAAGTTTTTGACATAAGCATCATGATTCTCATC
TGTCTTAACATGGTCACAATGATGGTGGAAACAGATGACCAGAGTGAATATGTGACTACCATTTTGTCACGCATCAAT
CTGGTGTTCATTGTGCTATTTACTGGAGAGTGTGTACTGAAACTCATCTCTCTACGCCATTATTATTTTACCATTGGA
TGGAATATTTTTGATTTTGTGGTTGTCATTCTCTCCATTGTAGGTATGTTTCTTGCCGAGCTGATAGAAAAGTATTTC
GTGTCCCCTACCCTGTTCCGAGTGATCCGTCTTGCTAGGATTGGCCGAATCCTACGTCTGATCAAAGGAGCAAAGGGG
ATCCGCACGCTGCTCTTTGCTTTGATGATGTCCCTTCCTGCGTTGTTTAACATCGGCCTCCTACTCTTCCTAGTCATG
TTCATCTACGCCATCTTTGGGATGTCCAACTTTGCCTATGTTAAGAGGGAAGTTGGGATCGATGACATGTTCAACTTT
GAGACCTTTGGCAACAGCATGATCTGCCTATTCCAAATTACAACCTCTGCTGGCTGGGATGGATTGCTAGCACCCATT
CTCAACAGTAAGCCACCCGACTGTGACCCTAATAAAGTTAACCCTGGAAGCTCAGTTAAGGGAGACTGTGGGAACCCA
TCTGTTGGAATTTTCTTTTTTGTCAGTTACATCATCATATCCTTCCTGGTTGTGGTGAACATGTACATCGCGGTCATC
CTGGAGAACTTCAGTGTTGCTACTGAAGAAAGTGCAGAGCCTCTGAGTGAGGATGACTTTGAGATGTTCTATGAGGTT
TGGGAGAAGTTTGATCCCGATGCAACTCAGTTCATGGAATTTGAAAAATTATCTCAGTTTGCAGCTGCGCTTGAACCG
CCTCTCAATCTGCCACAACCAAACAAACTCCAGCTCATTGCCATGGATTTGCCCATGGTGAGTGGTGACCGGATCCAC
TGTCTTGATATCTTATTTGCTTTTACAAAGCGGGTTCTAGGAGAGAGTGGAGAGATGGATGCTCTACGAATACAGATG
GAAGAGCGATTCATGGCTTCCAATCCTTCCAAGGTCTCCTATCAGCCAATCACTACTACTTTAAAACGAAAACAAGAG
GAAGTATCTGCTGTCATTATTCAGCGTGCTTACAGACGCCACCTTTTAAAGCGAACTGTAAAACAAGCTTCCTTTACG
TACAATAAAAACAAAATCAAAGGTGGGGCTAATCTTCTTATAAAAGAAGACATGATAATTGACAGAATAAATGAAAAC
TCTATTACAGAAAAAACTGATCTGACCATGTCCACTGCAGCTTGTCCACCTTCCTATGACCGGGTGACAAAGCCAATT
GTGGAAAAACATGAGCAAGAAGGCAAAGATGAAAAAGCCAAAGGGAAATAAATGAAAATAAATAAAAATAATTGGGTG
ACAAATTGTTTACAGCCTGTGAAGGTGATGTATTTTTATCAACAGGACTCCTTTAGGAGGTCAATGCCAAACTGACTG
TTTTTACACAAATCTCCTTAAGGTCAGTGCCTACAATAAGACAGTGACCCCTTGTCAGCAAACTGTGACTCTGTGTAA
AGGGGAGATGACCTTGACAGGAGGTTACTGTTCTCACTACCAGCTGACACTGCTGAAGATAAGATGCACAATGGCTAG
TCAGACTGTAGGGACCAGTTTCAAGGGGTGCAAACCTGTGATTTTGGGGTTGTTTAACATGAAACACTTTAGTGTAGT
AATTGTATCCACTGTTTGCATTTCAACTGCCACATTTGTCACATTTTTATGGAATCTGTTAGTGGATTCATCTTTTTG
TTAATCCATGTGTTTATTATATGTGACTATTTTTGTAAACGAAGTTTCTGTTGAGAAATAGGCTAAGGACCTCTATAA
CAGGTATGCCACCTGGGGGGTATGGCAACCACATGGCCCTCCCAGCTACACAAAGTCGTGGTTTGCATGAGGGCATGC
TGCACTTAGAGATCATGCATGAGAAAAAGTCACAAGAAAAACAAATTCTTAAATTTCACCATATTTCTGGGAGGGGTA
ATTGGGTGATAAGTGGAGGTGCTTTGTTGATCTTGTTTTGCGAAATCCAGCCCCTAGACCAAGTAGATTATTTGTGGG
TAGGCCAGTAAATCTTAGCAGGTGCAAACTTCATTCAAATGTTTGGAGTCATAAATGTTATGTTTCTTTTTGTTGTAT
TAAAAAAAAAACCTGAATAGTGAATATTGCCCCTCACCCTCCACCGCCAGAAGACTGAATTGACCAAAATTACTCTTT
ATAAATTTCTGCTTTTTCCTGCACTTTGTTTAGCCATCTTCGGCTCTCAGCAAGGTTGACACTGTATATGTTAATGAA
ATGCTATTTATTATGTAAATAGTCATTTTACCCTGTGGTGCACGTTTGAGCAAACAAATAATGACCTAAGCACAGTAT
TTATTGCATCAAATATGTACCACAAGAAATGTAGAGTGCAAGCTTTACACAGGTAATAAAATGTATTCTGTACCATTT
ATAGATAGTTTGGATGCTATCAATGCATGTTTATATTACCATGCTGCTGTATCTGGTTTCTCTCACTGCTCAGAATCT
CATTTATGAGAAACCATATGTCAGTGGTAAAGTCAAGGAAATTGTTCAACAGATCTCATTTATTTAAGTCATTAAGCA
ATAGTTTGCAGCACTTTAACAGCTTTTTGGTTATTTTTACATTTTAAGTGGATAACATATGGTATATAGCCAGACTGT
ACAGACATGTTTAAAAAAACACACTGCTTAACCTATTAAATATGTGTTTAGAATTTTATAAGCAAATATAAATACTGT
AAAAAGTCACTTTATTTTATTTTTCAGCATTATGTACATAAATATGAAGAGGAAATTATCTTCAGGTTGATATCACAA
TCACTTTTCTTACTTTCTGTCCATAGTACTTTTTCATGAAAGAAATTTGCTAAATAAGACATGAAAACAAGACTGGGT
AGTTGTAGATTTCTGCTTTTTAAATTACATTTGCTAATTTTAGATTATTTCACAATTTTAAGGAGCAAAATAGGTTCA
CGATTCATATCCAAATTATGCTTTGCAATTGGAAAAGGGTTTAAAATTTTATTTATATTTCTGGTAGTACCTGCACTA
ACTGAATTGAAGGTAGTGCTTATGTTATTTTTGTTCTTTTTTTCTGACTTCGGTTTATGTTTTCATTTCTTTGGAGTA
ATGCTGCTCTAGATTGTTCTAAATAGAATGTGGGCTTCATAATTTTTTTTTCCACAAAAACAGAGTAGTCAACTTATA
TAGTCAATTACATCAGGACATTTTGTGTTTCTTACAGAAGCAAACCATAGGCTCCTCTTTTCCTTAAAACTACTTAGA
TAAACTGTATTCGTGAACTGCATGCTGGAAAATGCTACTATTATGCTAAATAATGCTAACCAACATTTAAAATGTGCA
AAACTAATAAAGATTACATTTTTTATTTTATTGTTTGCCCAGTCACTTTTTGTTAACAGAATATTCTAATGATATGGA
GATTTTTTACATTACAAATTGGGGGAGAAGGGGAGCGCGCGCGCACACACACACACACACACACACACACACACACAC
ACACAGAGGCATACCCACGTTGACAACAAAACCTAGGGTAGATATGTCACTGGAGGTAGGGGGTAATGACCTCCCAGA
ATTACAAGCAGCAGGTGTGTTCTCTGTTAGGAGGAAGAACTGGTGTCAGAGGATAGCTAGTGATTCTAGGAGGAAGAG
AAGTATGGAAGCCAGAGTGATGGTGGATGACCCCTTGAGCTATGAAAAGAAACCCTTAAATCATCATTTAAAAATTTA
GAATTGCCATGTGTGTAGGATACTGTGTTTGCTCCTCCAGAGCCACTCTCTCTGCTTCTGCATCATTCTGTGTGTCCC
AGAAGGGTGACTTCTACACATTGCAAAAATGGGCTCTCCTACCTTTGAGCTCCCAATTGGTTTGGCCAATGAGAAGCA
CCAGTGGGAAAGCACCAGAGAGAGAAGATTGACATAGGAATATTTCTTCTCCAATTCCTTCTTTGCTGGGTTGGCACT
GGACTCATTCCTCCCCGAAAAGTCATACTCCAATCAGACTGCCCCTCATACAACTGAAGCTACTTTCTCTGGGGTCAG
GTAATCACTCCTCCCCTTGCTCCTTCAGGTCTGCTGCTGCATTGAGAGTGCTTTTGTATTCCTTGTAGCTTTCTCCTA
ACATTGCTGACACTTTTGTAAATGTCCCCTTCATGAAATTCTTCTATATGCCTCATTTCAGCATGCCATCTGTCTCCT
GCCTGGCTGACACAAGGTGATTCAACAGCTCATGAAAGTCAGCAGGAAGCAAAGATGTGCCTTGCTTCAGCTTGGGGT
CTTAATCTTGCTAACTTTTGCAGATAAAGAAAAACAGTAACTGGGGGAACCACAGTGAAGTCCAGTGCAGAATTCACA
GATATCATGGAAAGGTTACTCGGGTGGTCCAGATAGTAAAATTAACAGTCTAAATTAATCTATCTAAATTTCTGAGGA
ACGAGAAGCCTTCCCTTGTCATCAGGTGAAGCCAGAAGAGGGAATATAGCCTCAACCAGAAAAGGGACAGTAATTAAA
AGGCTTTTCCCATCCTTGTACAATGGACTGACTTTGCCTCTTCATAACATCACAATCCTAAAGCAACACAACAATTAA
TTCTGATATATTAGTAGCTGAAAAAAATTCCCATTTCCAACTAAGGTAGGTCAGAATTATAGGATAAACCCTGCAGAC
TTTTTATACTACCCATCCACGCCATTACTCACTGTTACCTTTCCAAATACAAAGAGAAGAACTGGTAAAACATAATCA
TATAAATCTCCATATTCATTTTGAAATATTTGGCATGATATTTTCTGTGCTAAAAAGTAATTATTCTTCAAAGAATGA
TGAGGTCATGTCAGTAAGACACAGGAACCAACTAGAAGGGGCTTCCCACTGGCCAAATCTGGGGCAAGTTGAGCATCA
AAATAAATGATAGTAAAAGATTATAATTCATTGAATAAGAATCAGCAAATACATACTGATGTAAGTAAATAAGGAAAA
GTACAAATCTGTTTCTTGCAGTTGAATGTTAATTAACAATTGTAGAAGAAATAACGGAGTTAGAAAAATCACTATTTG
GCAATCACCCTAATGACAATTGATTCATACAAGAATCATCAATGAGTATTAAAACTCATGGGTGAAAGTTTGATGAGG
AATAGGGTATTTATAGCATCTTAAAGTATCTCTTCTCTATTAAGTAGAAAATTTAAACAGAAGAAAGTATACTTTGGA
GAAATACAGCAGACAATACCTTCAAAGATATCATCAATTATGAGACCAACTGATACTATGTGCCTCCTGATAAGATAT
ACTGAAAGGGCCACATTACTTCTTGGTACACAGTCAAAATTTTAAAACCAGAATCTAACTACAAGGAAAATCAAATTG
AGGACACTCTATAAAATAAGTGGACTGAACTCCTTAAAAATGTCAATGTCATGAAAGACAAAGAAAGGCTAAAGAATT
CCATGAGGTCAAAGAACTATGACAACTAAACACAATTCTGGATGGAATATCAAATTAAAAAATAACAGATAAATAATA
TTATTGGGAAAGTTGAATAAATTTGAATATGGACTGTTTATTAGTTATTAGTATTATAATAGTGTTAATTTTCCTAAT
TTTGTTAAGACTAGTGTGCCTGTTCCATGAAAATAGAAAATGTTCTTATTCTCTGAAAATGCATGCTAAAGTATTTAG
GGGTGAATGCAACAATGTCTGCAGCTCATTCTTGAATCAGTTCAAAGAAAAATGAGTTACATTTATATATATATGTAT
GTATGTAAACAGACATAGATAAAAGTATAGATGTGTGTGTGTCTTTAGAAAGGGGAGGATTTTTTTTTTTTTTTTGCT
GTGTGTTACTGAAGTGCCTATGTCTGCGTGTTCACACTATCATATTTTGTATGCCCTGGACTTTATAATTTCTACCTT
CAAAATTAGATCTACTGTTGGTAATTAATTCAATATATACTGGTTTTTTAACTACTATTCTCATTTCCTAGCAGTAAT
CTTCCTGAAAAGTCACAGAAATGATTACATTCCTTGTTCTTCATAATAATCACTGTTTAATTAAAATAAGAATATTTT
AGAAAAGATCTGCGGCATAGTGGTTAAGACCCCAGTATTTGATGCTAAACAGATCTGATTTGGATAACAGAAGGTGGC
ACTTTGCTGTTTAAGCTGGGGACCAGACACTGTGGGTATAAATAGTAATTCCAAACACAGCTCCACAGAGCAGCACCC
TTATGACAAGGTTTTCATATGTCTATAGTTAAGCCAGAAAATTAAGAATAATGCCATAAATATTTATAAAGCTGAACA
TATCCAAGTTAAAGACCTTTATCCTGAAATTGTATCTTTTAGATTATTTTCTAAAGACTAATACCATTTAATGTTTAA
ATGTTCTTTGGAAATGATGGTGAGAATACGTGATAATGGGTCATTGGTTTTAATATTTTATTTAGCCAAGTGGAAAAT
TGGCAACCTGGTGTCGGTCCTCCCATTTGTATTTTACTGGTGCATGAAATCCAAAAGTCTAGTAACCATTGGGACAGA
CAACTCTACTGCATAAGTTTGTATGTTTGTATATCTGTATCACAAAGCCCAGACACTCGAACTATATAAACTTGTCGC
ACTAAAGACAGCAAATATGTCTGGTAATTGCATATTCTTCATGTGTGCACTGGAATTTCTTATTATATAAGAAAATAA
ATGTGTTTCTAAACCACCATGAATTGGGTACTGCTGATAGTACTATTCTTCCTGGCACATGGAAATATCCCACTGAGG
TTGTCAATCAATATTGATTTAATCAGTGTGGCAGCAAAGGCACTAGAGGAGGAAAATACTCTAGACAATCAAGTATAG
ATGGGAAAATTGCATCACCAAGAAATTCTTAAAATCACAAGTGGATAATTTGCAATATCAATTGATTTTACTCAACTG
CTTGAGTCCTTTATCATTCCGTTAGAACACTCCTCATATTTGCAGATAACTCAGCCCCTCACCTCAGCAACAAGCAAG
AGGACATCATTATAAACTTCCTCAATTTCTTTCCTATCCAGTGAAAAATATCTAAGTCCCTTTTCCCTTCCCTTTTCC
TTCCCTTCCCTTCCTTTTTCTTTCCTTCCATTCTCTTCCTTTTTCCTTCCTTTTTCTTTCCTTCCATTCTCTTCCTTT
TTCCTTCCTTTTTCTTACTTTACCTCCCCCTCCTACCTTTCCCTTTCTCCCTTCCTCCCTCTCTCCCTTCCTTCCTCT
CTCCCTCTCCTTCCTTCCTTCCTTCAATCCATACTTTTATATCTTTTCAGAGATCAATTTTTCCCATTCATTTCTACA
TTCTCAGATACCTTGCTCCATGACTTGTTCTACTTCTCTTCTATCTTTATTCTTTCCCTCTTGACTGATCCTTTTTGT
CTGCCTTTGCATTTTCAAAATCAGTTTTTCTAATCTTAAAAAATAACTTTGCTCAACCCTGATGCATCTTTATTCACT
GTGTCATTATCTCATTATTTTTAAAGGTTAGTTTCTAAAGCCAGGGCTCTATACTCACTACCTTCACTCTTGGTTAAA
ATTAAAAGAAATATAGGTCCCAGTGATATTTATGGGAAAATTTGACTGTGAATAATCTTGTTGATATAAACACTTTGG
GTTTTCAAAATGTTATGCTAGCTGTTAACTTTCCCAGTAACATTATGAGGGAAGGGCTGGAAAATACAAAAGGTAGAC
ATAGGAACCGAGAACACCACCCCTATGGAGCCAAGCTATACATACTCTGTCTCTGGTTATCATAAATACATCTCCTAT
ATTCAATGCTTGCTTAGAAAGGCATGATGTCCACGGAGCAATTCAATTGTTCACTGTGATGGGTTTTTTTGTTTTCTC
CTCATGGAATGCAACTTTTATTTAT
SEQ ID NO: 2 >XR_001738884.1 SCN1A [organism = Homo sapiens] [GeneID = 6323]
[transcript = X2]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGCAGGCAGCAACGGCAACTGCCTCAGA
ACATTCCAGAGAGCCCAGTGCAGCAGGCAGGCTCTCAGACAGCTCATCTGAAGCCTCTAAGTTGAGTTCCAAGAGTGC
TAAGGAAAGAAGAAATCGGAGGAAGAAAAGAAAACAGAAAGAGCAGTCTGGTGGGGAAGAGAAAGATGAGGATGAATT
CCAAAAATCTGAATCTGAGGACAGCATCAGGAGGAAAGGTTTTCGCTTCTCCATTGAAGGGAACCGATTGACATATGA
AAAGAGGTACTCCTCCCCACACCAGTCTTTGTTGAGCATCCGTGGCTCCCTATTTTCACCAAGGCGAAATAGCAGAAC
AAGCCTTTTCAGCTTTAGAGGGCGAGCAAAGGATGTGGGATCTGAGAACGACTTCGCAGATGATGAGCACAGCACCTT
TGAGGATAACGAGAGCCGTAGAGATTCCTTGTTTGTGCCCCGACGACACGGAGAGAGACGCAACAGCAACCTGAGTCA
GACCAGTAGGTCATCCCGGATGCTGGCAGTGTTTCCAGCGAATGGGAAGATGCACAGCACTGTGGATTGCAATGGTGT
GGTTTCCTTGGTTGGTGGACCTTCAGTTCCTACATCGCCTGTTGGACAGCTTCTGCCAGAGGGAACAACCACTGAAAC
TGAAATGAGAAAGAGAAGGTCAAGTTCTTTCCACGTTTCCATGGACTTTCTAGAAGATCCTTCCCAAAGGCAACGAGC
AATGAGTATAGCCAGCATTCTAACAAATACAGTAGAAGAACTTGAAGAATCCAGGCAGAAATGCCCACCCTGTTGGTA
TAAATTTTCCAACATATTCTTAATCTGGGACTGTTCTCCATATTGGTTAAAAGTGAAACATGTTGTCAACCTGGTTGT
GATGGACCCATTTGTTGACCTGGCCATCACCATCTGTATTGTCTTAAATACTCTTTTCATGGCCATGGAGCACTATCC
AATGACGGACCATTTCAATAATGTGCTTACAGTAGGAAACTTGGTTTTCACTGGGATCTTTACAGCAGAAATGTTTCT
GAAAATTATTGCCATGGATCCTTACTATTATTTCCAAGAAGGCTGGAATATCTTTGACGGTTTTATTGTGACGCTTAG
CCTGGTAGAACTTGGACTCGCCAATGTGGAAGGATTATCTGTTCTCCGTTCATTTCGATTGCTGCGAGTTTTCAAGTT
GGCAAAATCTTGGCCAACGTTAAATATGCTAATAAAGATCATCGGCAATTCCGTGGGGGCTCTGGGAAATTTAACCCT
CGTCTTGGCCATCATCGTCTTCATTTTTGCCGTGGTCGGCATGCAGCTCTTTGGTAAAAGCTACAAAGATTGTGTCTG
CAAGATCGCCAGTGATTGTCAACTCCCACGCTGGCACATGAATGACTTCTTCCACTCCTTCCTGATTGTGTTCCGCGT
GCTGTGTGGGGAGTGGATAGAGACCATGTGGGACTGTATGGAGGTTGCTGGTCAAGCCATGTGCCTTACTGTCTTCAT
GATGGTCATGGTGATTGGAAACCTAGTGGTCCTGAATCTCTTTCTGGCCTTGCTTCTGAGCTCATTTAGTGCAGACAA
CCTTGCAGCCACTGATGATGATAATGAAATGAATAATCTCCAAATTGCTGTGGATAGGATGCACAAAGGAGTAGCTTA
TGTGAAAAGAAAAATATATGAATTTATTCAACAGTCCTTCATTAGGAAACAAAAGATTTTAGATGAAATTAAACCACT
TGATGATCTAAACAACAAGAAAGACAGTTGTATGTCCAATCATACAGCAGAAATTGGGAAAGATCTTGACTATCTTAA
AGATGTAAATGGAACTACAAGTGGTATAGGAACTGGCAGCAGTGTTGAAAAATACATTATTGATGAAAGTGATTACAT
GTCATTCATAAACAACCCCAGTCTTACTGTGACTGTACCAATTGCTGTAGGAGAATCTGACTTTGAAAATTTAAACAC
GGAAGACTTTAGTAGTGAATCGGATCTGGAAGAAAGCAAAGAGAAACTGAATGAAAGCAGTAGCTCATCAGAAGGTAG
CACTGTGGACATCGGCGCACCTGTAGAAGAACAGCCCGTAGTGGAACCTGAAGAAACTCTTGAACCAGAAGCTTGTTT
CACTGAAGGCTGTGTACAAAGATTCAAGTGTTGTCAAATCAATGTGGAAGAAGGCAGAGGAAAACAATGGTGGAACCT
GAGAAGGACGTGTTTCCGAATAGTTGAACATAACTGGTTTGAGACCTTCATTGTTTTCATGATTCTCCTTAGTAGTGG
TGCTCTGGCATTTGAAGATATATATATTGATCAGCGAAAGACGATTAAGACGATGTTGGAATATGCTGACAAGGTTTT
CACTTACATTTTCATTCTGGAAATGCTTCTAAAATGGGTGGCATATGGCTATCAAACATATTTCACCAATGCCTGGTG
TTGGCTGGACTTCTTAATTGTTGATGTTTCATTGGTCAGTTTAACAGCAAATGCCTTGGGTTACTCAGAACTTGGAGC
CATCAAATCTCTCAGGACACTAAGAGCTCTGAGACCTCTAAGAGCCTTATCTCGATTTGAAGGGATGAGGGATAATCT
TGCTCCAACTTGGATGGGGTGGAGCGCTGGTTCCTCCCCTGAGCCCTTTATTATGGGTGGTTGTGAATGCCCTTTTAG
GAGCAATTCCATCCATCATGAATGTGCTTCTGGTTTGTCTTATATTCTGGCTAATTTTCAGCATCATGGGCGTAAATT
TGTTTGCTGGCAAATTCTACCACTGTATTAACACCACAACTGGTGACAGGTTTGACATCGAAGACGTGAATAATCATA
CTGATTGCCTAAAACTAATAGAAAGAAATGAGACTGCTCGATGGAAAAATGTGAAAGTAAACTTTGATAATGTAGGAT
TTGGGTATCTCTCTTTGCTTCAAGTTGCCACATTCAAAGGATGGATGGATATAATGTATGCAGCAGTTGATTCCAGAA
ATGTGGAACTCCAGCCTAAGTATGAAGAAAGTCTGTACATGTATCTTTACTTTGTTATTTTCATCATCTTTGGGTCCT
TCTTCACCTTGAACCTGTTTATTGGTGTCATCATAGATAATTTCAACCAGCAGAAAAAGAAGTTTGGAGGTCAAGACA
TCTTTATGACAGAAGAACAGAAGAAATACTATAATGCAATGAAAAAATTAGGATCGAAAAAACCGCAAAAGCCTATAC
CTCGACCAGGAAACAAATTTCAAGGAATGGTCTTTGACTTCGTAACCAGACAAGTTTTTGACATAAGCATCATGATTC
TCATCTGTCTTAACATGGTCACAATGATGGTGGAAACAGATGACCAGAGTGAATATGTGACTACCATTTTGTCACGCA
TCAATCTGGTGTTCATTGTGCTATTTACTGGAGAGTGTGTACTGAAACTCATCTCTCTACGCCATTATTATTTTACCA
TTGGATGGAATATTTTTGATTTTGTGGTTGTCATTCTCTCCATTGTAGGTATGTTTCTTGCCGAGCTGATAGAAAAGT
ATTTCGTGTCCCCTACCCTGTTCCGAGTGATCCGTCTTGCTAGGATTGGCCGAATCCTACGTCTGATCAAAGGAGCAA
AGGGGATCCGCACGCTGCTCTTTGCTTTGATGATGTCCCTTCCTGCGTTGTTTAACATCGGCCTCCTACTCTTCCTAG
TCATGTTCATCTACGCCATCTTTGGGATGTCCAACTTTGCCTATGTTAAGAGGGAAGTTGGGATCGATGACATGTTCA
ACTTTGAGACCTTTGGCAACAGCATGATCTGCCTATTCCAAATTACAACCTCTGCTGGCTGGGATGGATTGCTAGCAC
CCATTCTCAACAGTAAGCCACCCGACTGTGACCCTAATAAAGTTAACCCTGGAAGCTCAGTTAAGGGAGACTGTGGGA
ACCCATCTGTTGGAATTTTCTTTTTTGTCAGTTACATCATCATATCCTTCCTGGTTGTGGTGAACATGTACATCGCGG
TCATCCTGGAGAACTTCAGTGTTGCTACTGAAGAAAGTGCAGAGCCTCTGAGTGAGGATGACTTTGAGATGTTCTATG
AGGTTTGGGAGAAGTTTGATCCCGATGCAACTCAGTTCATGGAATTTGAAAAATTATCTCAGTTTGCAGCTGCGCTTG
AACCGCCTCTCAATCTGCCACAACCAAACAAACTCCAGCTCATTGCCATGGATTTGCCCATGGTGAGTGGTGACCGGA
TCCACTGTCTTGATATCTTATTTGCTTTTACAAAGCGGGTTCTAGGAGAGAGTGGAGAGATGGATGCTCTACGAATAC
AGATGGAAGAGCGATTCATGGCTTCCAATCCTTCCAAGGTCTCCTATCAGCCAATCACTACTACTTTAAAACGAAAAC
AAGAGGAAGTATCTGCTGTCATTATTCAGCGTGCTTACAGACGCCACCTTTTAAAGCGAACTGTAAAACAAGCTTCCT
TTACGTACAATAAAAACAAAATCAAAGGTGGGGCTAATCTTCTTATAAAAGAAGACATGATAATTGACAGAATAAATG
AAAACTCTATTACAGAAAAAACTGATCTGACCATGTCCACTGCAGCTTGTCCACCTTCCTATGACCGGGTGACAAAGC
CAATTGTGGAAAAACATGAGCAAGAAGGCAAAGATGAAAAAGCCAAAGGGAAATAAATGAAAATAAATAAAAATAATT
GGGTGACAAATTGTTTACAGCCTGTGAAGGTGATGTATTTTTATCAACAGGACTCCTTTAGGAGGTCAATGCCAAACT
GACTGTTTTTACACAAATCTCCTTAAGGTCAGTGCCTACAATAAGACAGTGACCCCTTGTCAGCAAACTGTGACTCTG
TGTAAAGGGGAGATGACCTTGACAGGAGGTTACTGTTCTCACTACCAGCTGACACTGCTGAAGATAAGATGCACAATG
GCTAGTCAGACTGTAGGGACCAGTTTCAAGGGGTGCAAACCTGTGATTTTGGGGTTGTTTAACATGAAACACTTTAGT
GTAGTAATTGTATCCACTGTTTGCATTTCAACTGCCACATTTGTCACATTTTTATGGAATCTGTTAGTGGATTCATCT
TTTTGTTAATCCATGTGTTTATTATATGTGACTATTTTTGTAAACGAAGTTTCTGTTGAGAAATAGGCTAAGGACCTC
TATAACAGGTATGCCACCTGGGGGGTATGGCAACCACATGGCCCTCCCAGCTACACAAAGTCGTGGTTTGCATGAGGG
CATGCTGCACTTAGAGATCATGCATGAGAAAAAGTCACAAGAAAAACAAATTCTTAAATTTCACCATATTTCTGGGAG
GGGTAATTGGGTGATAAGTGGAGGTGCTTTGTTGATCTTGTTTTGCGAAATCCAGCCCCTAGACCAAGTAGATTATTT
GTGGGTAGGCCAGTAAATCTTAGCAGGTGCAAACTTCATTCAAATGTTTGGAGTCATAAATGTTATGTTTCTTTTTGT
TGTATTAAAAAAAAAACCTGAATAGTGAATATTGCCCCTCACCCTCCACCGCCAGAAGACTGAATTGACCAAAATTAC
TCTTTATAAATTTCTGCTTTTTCCTGCACTTTGTTTAGCCATCTTCGGCTCTCAGCAAGGTTGACACTGTATATGTTA
ATGAAATGCTATTTATTATGTAAATAGTCATTTTACCCTGTGGTGCACGTTTGAGCAAACAAATAATGACCTAAGCAC
AGTATTTATTGCATCAAATATGTACCACAAGAAATGTAGAGTGCAAGCTTTACACAGGTAATAAAATGTATTCTGTAC
CATTTATAGATAGTTTGGATGCTATCAATGCATGTTTATATTACCATGCTGCTGTATCTGGTTTCTCTCACTGCTCAG
AATCTCATTTATGAGAAACCATATGTCAGTGGTAAAGTCAAGGAAATTGTTCAACAGATCTCATTTATTTAAGTCATT
AAGCAATAGTTTGCAGCACTTTAACAGCTTTTTGGTTATTTTTACATTTTAAGTGGATAACATATGGTATATAGCCAG
ACTGTACAGACATGTTTAAAAAAACACACTGCTTAACCTATTAAATATGTGTTTAGAATTTTATAAGCAAATATAAAT
ACTGTAAAAAGTCACTTTATTTTATTTTTCAGCATTATGTACATAAATATGAAGAGGAAATTATCTTCAGGTTGATAT
CACAATCACTTTTCTTACTTTCTGTCCATAGTACTTTTTCATGAAAGAAATTTGCTAAATAAGACATGAAAACAAGAC
TGGGTAGTTGTAGATTTCTGCTTTTTAAATTACATTTGCTAATTTTAGATTATTTCACAATTTTAAGGAGCAAAATAG
GTTCACGATTCATATCCAAATTATGCTTTGCAATTGGAAAAGGGTTTAAAATTTTATTTATATTTCTGGTAGTACCTG
CACTAACTGAATTGAAGGTAGTGCTTATGTTATTTTTGTTCTTTTTTTCTGACTTCGGTTTATGTTTTCATTTCTTTG
GAGTAATGCTGCTCTAGATTGTTCTAAATAGAATGTGGGCTTCATAATTTTTTTTTCCACAAAAACAGAGTAGTCAAC
TTATATAGTCAATTACATCAGGACATTTTGTGTTTCTTACAGAAGCAAACCATAGGCTCCTCTTTTCCTTAAAACTAC
TTAGATAAACTGTATTCGTGAACTGCATGCTGGAAAATGCTACTATTATGCTAAATAATGCTAACCAACATTTAAAAT
GTGCAAAACTAATAAAGATTACATTTTTTATTTTA
SEQ ID NO: 3 >XR_001738883.1 SCN1A [organism = Homo sapiens] [GeneID = 6323]
[transcript = X1]
AGAGTGAGGCGAGGATGAAGCCGAGAGGATACTGCAGAGGTCTCTGGTGCATGTGTGTATGTGTGCGTTTGTGTGTGT
TTGTGTGTCTGTGTGTTCTGCCCCAGTGAGACTGCAGCCCTTGTAAATACTTTGACACCTTTTGCAAGAAGGAATCTG
AACAATTGCAACTGAAGGCACATTGTTATCATCTCGTCTTTGGGTGATGCTGTTCCTCACTGCAGATGGATAATTTTC
CTTTTAATCAGGTTTGGGCAATTATGAATAAGGCTGCTGTATACATCCGTGTGCAGGATTTTGTGTGGACATAAGTTT
TCAACTCCTTTGGTTAAATCCTAAGGAATTTCATATGCAGAATAAATGGTAATTAAAATGTGCAGGATGACAAGATGG
AGCAAACAGTGCTTGTACCACCAGGACCTGACAGCTTCAACTTCTTCACCAGAGAATCTCTTGCGGCTATTGAAAGAC
GCATTGCAGAAGAAAAGGCAAAGAATCCCAAACCAGACAAAAAAGATGACGACGAAAATGGCCCAAAGCCAAATAGTG
ACTTGGAAGCTGGAAAGAACCTTCCATTTATTTATGGAGACATTCCTCCAGAGATGGTGTCAGAGCCCCTGGAGGACC
TGGACCCCTACTATATCAATAAGAAAACTTTTATAGTATTGAATAAAGGGAAGGCCATCTTCCGGTTCAGTGCCACCT
CTGCCCTGTACATTTTAACTCCCTTCAATCCTCTTAGGAAAATAGCTATTAAGATTTTGGTACATTCATTATTCAGCA
TGCTAATTATGTGCACTATTTTGACAAACTGTGTGTTTATGACAATGAGTAACCCTCCTGATTGGACAAAGAATGTAG
AATACACCTTCACAGGAATATATACTTTTGAATCACTTATAAAAATTATTGCAAGGGGATTCTGTTTAGAAGATTTTA
CTTTCCTTCGGGATCCATGGAACTGGCTCGATTTCACTGTCATTACATTTGCGTACGTCACAGAGTTTGTGGACCTGG
GCAATGTCTCGGCATTGAGAACATTCAGAGTTCTCCGAGCATTGAAGACGATTTCAGTCATTCCAGGCCTGAAAACCA
TTGTGGGAGCCCTGATCCAGTCTGTGAAGAAGCTCTCAGATGTAATGATCCTGACTGTGTTCTGTCTGAGCGTATTTG
CTCTAATTGGGCTGCAGCTGTTCATGGGCAACCTGAGGAATAAATGTATACAATGGCCTCCCACCAATGCTTCCTTGG
AGGAACATAGTATAGAAAAGAATATAACTGTGAATTATAATGGTACACTTATAAATGAAACTGTCTTTGAGTTTGACT
GGAAGTCATATATTCAAGATTCAAGATATCATTATTTCCTGGAGGGTTTTTTAGATGCACTACTATGTGGAAATAGCT
CTGATGCAGGCCAATGTCCAGAGGGATATATGTGTGTGAAAGCTGGTAGAAATCCCAATTATGGCTACACAAGCTTTG
ATACCTTCAGTTGGGCTTTTTTGTCCTTGTTTCGACTAATGACTCAGGACTTCTGGGAAAATCTTTATCAACTGACAT
TACGTGCTGCTGGGAAAACGTACATGATATTTTTTGTATTGGTCATTTTCTTGGGCTCATTCTACCTAATAAATTTGA
TCCTGGCTGTGGTGGCCATGGCCTACGAGGAACAGAATCAGGCCACCTTGGAAGAAGCAGAACAGAAAGAGGCCGAAT
TTCAGCAGATGATTGAACAGCTTAAAAAGCAACAGGAGGCAGCTCAGCAGGCAGCAACGGCAACTGCCTCAGAACATT
CCAGAGAGCCCAGTGCAGCAGGCAGGCTCTCAGACAGCTCATCTGAAGCCTCTAAGTTGAGTTCCAAGAGTGCTAAGG
AAAGAAGAAATCGGAGGAAGAAAAGAAAACAGAAAGAGCAGTCTGGTGGGGAAGAGAAAGATGAGGATGAATTCCAAA
AATCTGAATCTGAGGACAGCATCAGGAGGAAAGGTTTTCGCTTCTCCATTGAAGGGAACCGATTGACATATGAAAAGA
GGTACTCCTCCCCACACCAGTCTTTGTTGAGCATCCGTGGCTCCCTATTTTCACCAAGGCGAAATAGCAGAACAAGCC
TTTTCAGCTTTAGAGGGCGAGCAAAGGATGTGGGATCTGAGAACGACTTCGCAGATGATGAGCACAGCACCTTTGAGG
ATAACGAGAGCCGTAGAGATTCCTTGTTTGTGCCCCGACGACACGGAGAGAGACGCAACAGCAACCTGAGTCAGACCA
GTAGGTCATCCCGGATGCTGGCAGTGTTTCCAGCGAATGGGAAGATGCACAGCACTGTGGATTGCAATGGTGTGGTTT
CCTTGGTTGGTGGACCTTCAGTTCCTACATCGCCTGTTGGACAGCTTCTGCCAGAGGTGATAATAGATAAGCCAGCTA
CTGATGACAATGGAACAACCACTGAAACTGAAATGAGAAAGAGAAGGTCAAGTTCTTTCCACGTTTCCATGGACTTTC
TAGAAGATCCTTCCCAAAGGCAACGAGCAATGAGTATAGCCAGCATTCTAACAAATACAGTAGAAGAACTTGAAGAAT
CCAGGCAGAAATGCCCACCCTGTTGGTATAAATTTTCCAACATATTCTTAATCTGGGACTGTTCTCCATATTGGTTAA
AAGTGAAACATGTTGTCAACCTGGTTGTGATGGACCCATTTGTTGACCTGGCCATCACCATCTGTATTGTCTTAAATA
CTCTTTTCATGGCCATGGAGCACTATCCAATGACGGACCATTTCAATAATGTGCTTACAGTAGGAAACTTGGTTTTCA
CTGGGATCTTTACAGCAGAAATGTTTCTGAAAATTATTGCCATGGATCCTTACTATTATTTCCAAGAAGGCTGGAATA
TCTTTGACGGTTTTATTGTGACGCTTAGCCTGGTAGAACTTGGACTCGCCAATGTGGAAGGATTATCTGTTCTCCGTT
CATTTCGATTGCTGCGAGTTTTCAAGTTGGCAAAATCTTGGCCAACGTTAAATATGCTAATAAAGATCATCGGCAATT
CCGTGGGGGCTCTGGGAAATTTAACCCTCGTCTTGGCCATCATCGTCTTCATTTTTGCCGTGGTCGGCATGCAGCTCT
TTGGTAAAAGCTACAAAGATTGTGTCTGCAAGATCGCCAGTGATTGTCAACTCCCACGCTGGCACATGAATGACTTCT
TCCACTCCTTCCTGATTGTGTTCCGCGTGCTGTGTGGGGAGTGGATAGAGACCATGTGGGACTGTATGGAGGTTGCTG
GTCAAGCCATGTGCCTTACTGTCTTCATGATGGTCATGGTGATTGGAAACCTAGTGGTCCTGAATCTCTTTCTGGCCT
TGCTTCTGAGCTCATTTAGTGCAGACAACCTTGCAGCCACTGATGATGATAATGAAATGAATAATCTCCAAATTGCTG
TGGATAGGATGCACAAAGGAGTAGCTTATGTGAAAAGAAAAATATATGAATTTATTCAACAGTCCTTCATTAGGAAAC
AAAAGATTTTAGATGAAATTAAACCACTTGATGATCTAAACAACAAGAAAGACAGTTGTATGTCCAATCATACAGCAG
AAATTGGGAAAGATCTTGACTATCTTAAAGATGTAAATGGAACTACAAGTGGTATAGGAACTGGCAGCAGTGTTGAAA
AATACATTATTGATGAAAGTGATTACATGTCATTCATAAACAACCCCAGTCTTACTGTGACTGTACCAATTGCTGTAG
GAGAATCTGACTTTGAAAATTTAAACACGGAAGACTTTAGTAGTGAATCGGATCTGGAAGAAAGCAAAGAGAAACTGA
ATGAAAGCAGTAGCTCATCAGAAGGTAGCACTGTGGACATCGGCGCACCTGTAGAAGAACAGCCCGTAGTGGAACCTG
AAGAAACTCTTGAACCAGAAGCTTGTTTCACTGAAGGCTGTGTACAAAGATTCAAGTGTTGTCAAATCAATGTGGAAG
AAGGCAGAGGAAAACAATGGTGGAACCTGAGAAGGACGTGTTTCCGAATAGTTGAACATAACTGGTTTGAGACCTTCA
TTGTTTTCATGATTCTCCTTAGTAGTGGTGCTCTGGCATTTGAAGATATATATATTGATCAGCGAAAGACGATTAAGA
CGATGTTGGAATATGCTGACAAGGTTTTCACTTACATTTTCATTCTGGAAATGCTTCTAAAATGGGTGGCATATGGCT
ATCAAACATATTTCACCAATGCCTGGTGTTGGCTGGACTTCTTAATTGTTGATGTTTCATTGGTCAGTTTAACAGCAA
ATGCCTTGGGTTACTCAGAACTTGGAGCCATCAAATCTCTCAGGACACTAAGAGCTCTGAGACCTCTAAGAGCCTTAT
CTCGATTTGAAGGGATGAGGGATAATCTTGCTCCAACTTGGATGGGGTGGAGCGCTGGTTCCTCCCCTGAGCCCTTTA
TTATGGGTGGTTGTGAATGCCCTTTTAGGAGCAATTCCATCCATCATGAATGTGCTTCTGGTTTGTCTTATATTCTGG
CTAATTTTCAGCATCATGGGCGTAAATTTGTTTGCTGGCAAATTCTACCACTGTATTAACACCACAACTGGTGACAGG
TTTGACATCGAAGACGTGAATAATCATACTGATTGCCTAAAACTAATAGAAAGAAATGAGACTGCTCGATGGAAAAAT
GTGAAAGTAAACTTTGATAATGTAGGATTTGGGTATCTCTCTTTGCTTCAAGTTGCCACATTCAAAGGATGGATGGAT
ATAATGTATGCAGCAGTTGATTCCAGAAATGTGGAACTCCAGCCTAAGTATGAAGAAAGTCTGTACATGTATCTTTAC
TTTGTTATTTTCATCATCTTTGGGTCCTTCTTCACCTTGAACCTGTTTATTGGTGTCATCATAGATAATTTCAACCAG
CAGAAAAAGAAGTTTGGAGGTCAAGACATCTTTATGACAGAAGAACAGAAGAAATACTATAATGCAATGAAAAAATTA
GGATCGAAAAAACCGCAAAAGCCTATACCTCGACCAGGAAACAAATTTCAAGGAATGGTCTTTGACTTCGTAACCAGA
CAAGTTTTTGACATAAGCATCATGATTCTCATCTGTCTTAACATGGTCACAATGATGGTGGAAACAGATGACCAGAGT
GAATATGTGACTACCATTTTGTCACGCATCAATCTGGTGTTCATTGTGCTATTTACTGGAGAGTGTGTACTGAAACTC
ATCTCTCTACGCCATTATTATTTTACCATTGGATGGAATATTTTTGATTTTGTGGTTGTCATTCTCTCCATTGTAGGT
ATGTTTCTTGCCGAGCTGATAGAAAAGTATTTCGTGTCCCCTACCCTGTTCCGAGTGATCCGTCTTGCTAGGATTGGC
CGAATCCTACGTCTGATCAAAGGAGCAAAGGGGATCCGCACGCTGCTCTTTGCTTTGATGATGTCCCTTCCTGCGTTG
TTTAACATCGGCCTCCTACTCTTCCTAGTCATGTTCATCTACGCCATCTTTGGGATGTCCAACTTTGCCTATGTTAAG
AGGGAAGTTGGGATCGATGACATGTTCAACTTTGAGACCTTTGGCAACAGCATGATCTGCCTATTCCAAATTACAACC
TCTGCTGGCTGGGATGGATTGCTAGCACCCATTCTCAACAGTAAGCCACCCGACTGTGACCCTAATAAAGTTAACCCT
GGAAGCTCAGTTAAGGGAGACTGTGGGAACCCATCTGTTGGAATTTTCTTTTTTGTCAGTTACATCATCATATCCTTC
CTGGTTGTGGTGAACATGTACATCGCGGTCATCCTGGAGAACTTCAGTGTTGCTACTGAAGAAAGTGCAGAGCCTCTG
AGTGAGGATGACTTTGAGATGTTCTATGAGGTTTGGGAGAAGTTTGATCCCGATGCAACTCAGTTCATGGAATTTGAA
AAATTATCTCAGTTTGCAGCTGCGCTTGAACCGCCTCTCAATCTGCCACAACCAAACAAACTCCAGCTCATTGCCATG
GATTTGCCCATGGTGAGTGGTGACCGGATCCACTGTCTTGATATCTTATTTGCTTTTACAAAGCGGGTTCTAGGAGAG
AGTGGAGAGATGGATGCTCTACGAATACAGATGGAAGAGCGATTCATGGCTTCCAATCCTTCCAAGGTCTCCTATCAG
CCAATCACTACTACTTTAAAACGAAAACAAGAGGAAGTATCTGCTGTCATTATTCAGCGTGCTTACAGACGCCACCTT
TTAAAGCGAACTGTAAAACAAGCTTCCTTTACGTACAATAAAAACAAAATCAAAGGTGGGGCTAATCTTCTTATAAAA
GAAGACATGATAATTGACAGAATAAATGAAAACTCTATTACAGAAAAAACTGATCTGACCATGTCCACTGCAGCTTGT
CCACCTTCCTATGACCGGGTGACAAAGCCAATTGTGGAAAAACATGAGCAAGAAGGCAAAGATGAAAAAGCCAAAGGG
AAATAAATGAAAATAAATAAAAATAATTGGGTGACAAATTGTTTACAGCCTGTGAAGGTGATGTATTTTTATCAACAG
GACTCCTTTAGGAGGTCAATGCCAAACTGACTGTTTTTACACAAATCTCCTTAAGGTCAGTGCCTACAATAAGACAGT
GACCCCTTGTCAGCAAACTGTGACTCTGTGTAAAGGGGAGATGACCTTGACAGGAGGTTACTGTTCTCACTACCAGCT
GACACTGCTGAAGATAAGATGCACAATGGCTAGTCAGACTGTAGGGACCAGTTTCAAGGGGTGCAAACCTGTGATTTT
GGGGTTGTTTAACATGAAACACTTTAGTGTAGTAATTGTATCCACTGTTTGCATTTCAACTGCCACATTTGTCACATT
TTTATGGAATCTGTTAGTGGATTCATCTTTTTGTTAATCCATGTGTTTATTATATGTGACTATTTTTGTAAACGAAGT
TTCTGTTGAGAAATAGGCTAAGGACCTCTATAACAGGTATGCCACCTGGGGGGTATGGCAACCACATGGCCCTCCCAG
CTACACAAAGTCGTGGTTTGCATGAGGGCATGCTGCACTTAGAGATCATGCATGAGAAAAAGTCACAAGAAAAACAAA
TTCTTAAATTTCACCATATTTCTGGGAGGGGTAATTGGGTGATAAGTGGAGGTGCTTTGTTGATCTTGTTTTGCGAAA
TCCAGCCCCTAGACCAAGTAGATTATTTGTGGGTAGGCCAGTAAATCTTAGCAGGTGCAAACTTCATTCAAATGTTTG
GAGTCATAAATGTTATGTTTCTTTTTGTTGTATTAAAAAAAAAACCTGAATAGTGAATATTGCCCCTCACCCTCCACC
GCCAGAAGACTGAATTGACCAAAATTACTCTTTATAAATTTCTGCTTTTTCCTGCACTTTGTTTAGCCATCTTCGGCT
CTCAGCAAGGTTGACACTGTATATGTTAATGAAATGCTATTTATTATGTAAATAGTCATTTTACCCTGTGGTGCACGT
TTGAGCAAACAAATAATGACCTAAGCACAGTATTTATTGCATCAAATATGTACCACAAGAAATGTAGAGTGCAAGCTT
TACACAGGTAATAAAATGTATTCTGTACCATTTATAGATAGTTTGGATGCTATCAATGCATGTTTATATTACCATGCT
GCTGTATCTGGTTTCTCTCACTGCTCAGAATCTCATTTATGAGAAACCATATGTCAGTGGTAAAGTCAAGGAAATTGT
TCAACAGATCTCATTTATTTAAGTCATTAAGCAATAGTTTGCAGCACTTTAACAGCTTTTTGGTTATTTTTACATTTT
AAGTGGATAACATATGGTATATAGCCAGACTGTACAGACATGTTTAAAAAAACACACTGCTTAACCTATTAAATATGT
GTTTAGAATTTTATAAGCAAATATAAATACTGTAAAAAGTCACTTTATTTTATTTTTCAGCATTATGTACATAAATAT
GAAGAGGAAATTATCTTCAGGTTGATATCACAATCACTTTTCTTACTTTCTGTCCATAGTACTTTTTCATGAAAGAAA
TTTGCTAAATAAGACATGAAAACAAGACTGGGTAGTTGTAGATTTCTGCTTTTTAAATTACATTTGCTAATTTTAGAT
TATTTCACAATTTTAAGGAGCAAAATAGGTTCACGATTCATATCCAAATTATGCTTTGCAATTGGAAAAGGGTTTAAA
ATTTTATTTATATTTCTGGTAGTACCTGCACTAACTGAATTGAAGGTAGTGCTTATGTTATTTTTGTTCTTTTTTTCT
GACTTCGGTTTATGTTTTCATTTCTTTGGAGTAATGCTGCTCTAGATTGTTCTAAATAGAATGTGGGCTTCATAATTT
TTTTTTCCACAAAAACAGAGTAGTCAACTTATATAGTCAATTACATCAGGACATTTTGTGTTTCTTACAGAAGCAAAC
CATAGGCTCCTCTTTTCCTTAAAACTACTTAGATAAACTGTATTCGTGAACTGCATGCTGGAAAATGCTACTATTATG
CTAAATAATGCTAACCAACATTTAAAATGTGCAAAACTAATAAAGATTACATTTTTTATTTTA
SEQ ID NO: 4 >NM_001353949.2 SCN1A [organism = Homo sapiens] [GeneID = 6323]
[transcript = 6]
AAGTGTAGGAGACACACTGCTGGCCTGTGGAAACTCATGGAACTGTTCCTCCAGATTAACACTTCAGGGGTTATGGAA
GCTGGAGGAAGCTGAGCTTTTACTACATCTTTTGGGGGAATTTCATATGCAGAATAAATGGTAATTAAAATGTGCAGG
ATGACAAGATGGAGCAAACAGTGCTTGTACCACCAGGACCTGACAGCTTCAACTTCTTCACCAGAGAATCTCTTGCGG
CTATTGAAAGACGCATTGCAGAAGAAAAGGCAAAGAATCCCAAACCAGACAAAAAAGATGACGACGAAAATGGCCCAA
AGCCAAATAGTGACTTGGAAGCTGGAAAGAACCTTCCATTTATTTATGGAGACATTCCTCCAGAGATGGTGTCAGAGC
CCCTGGAGGACCTGGACCCCTACTATATCAATAAGAAAACTTTTATAGTATTGAATAAAGGGAAGGCCATCTTCCGGT
TCAGTGCCACCTCTGCCCTGTACATTTTAACTCCCTTCAATCCTCTTAGGAAAATAGCTATTAAGATTTTGGTACATT
CATTATTCAGCATGCTAATTATGTGCACTATTTTGACAAACTGTGTGTTTATGACAATGAGTAACCCTCCTGATTGGA
CAAAGAATGTAGAATACACCTTCACAGGAATATATACTTTTGAATCACTTATAAAAATTATTGCAAGGGGATTCTGTT
TAGAAGATTTTACTTTCCTTCGGGATCCATGGAACTGGCTCGATTTCACTGTCATTACATTTGCGTACGTCACAGAGT
TTGTGGACCTGGGCAATGTCTCGGCATTGAGAACATTCAGAGTTCTCCGAGCATTGAAGACGATTTCAGTCATTCCAG
GCCTGAAAACCATTGTGGGAGCCCTGATCCAGTCTGTGAAGAAGCTCTCAGATGTAATGATCCTGACTGTGTTCTGTC
TGAGCGTATTTGCTCTAATTGGGCTGCAGCTGTTCATGGGCAACCTGAGGAATAAATGTATACAATGGCCTCCCACCA
ATGCTTCCTTGGAGGAACATAGTATAGAAAAGAATATAACTGTGAATTATAATGGTACACTTATAAATGAAACTGTCT
TTGAGTTTGACTGGAAGTCATATATTCAAGATTCAAGATATCATTATTTCCTGGAGGGTTTTTTAGATGCACTACTAT
GTGGAAATAGCTCTGATGCAGGCCAATGTCCAGAGGGATATATGTGTGTGAAAGCTGGTAGAAATCCCAATTATGGCT
ACACAAGCTTTGATACCTTCAGTTGGGCTTTTTTGTCCTTGTTTCGACTAATGACTCAGGACTTCTGGGAAAATCTTT
ATCAACTGACATTACGTGCTGCTGGGAAAACGTACATGATATTTTTTGTATTGGTCATTTTCTTGGGCTCATTCTACC
TAATAAATTTGATCCTGGCTGTGGTGGCCATGGCCTACGAGGAACAGAATCAGGCCACCTTGGAAGAAGCAGAACAGA
AAGAGGCCGAATTTCAGCAGATGATTGAACAGCTTAAAAAGCAACAGGAGGCAGCTCAGCAGGCAGCAACGGCAACTG
CCTCAGAACATTCCAGAGAGCCCAGTGCAGCAGGCAGGCTCTCAGACAGCTCATCTGAAGCCTCTAAGTTGAGTTCCA
AGAGTGCTAAGGAAAGAAGAAATCGGAGGAAGAAAAGAAAACAGAAAGAGCAGTCTGGTGGGGAAGAGAAAGATGAGG
ATGAATTCCAAAAATCTGAATCTGAGGACAGCATCAGGAGGAAAGGTTTTCGCTTCTCCATTGAAGGGAACCGATTGA
CATATGAAAAGAGGTACTCCTCCCCACACCAGTCTTTGTTGAGCATCCGTGGCTCCCTATTTTCACCAAGGCGAAATA
GCAGAACAAGCCTTTTCAGCTTTAGAGGGCGAGCAAAGGATGTGGGATCTGAGAACGACTTCGCAGATGATGAGCACA
GCACCTTTGAGGATAACGAGAGCCGTAGAGATTCCTTGTTTGTGCCCCGACGACACGGAGAGAGACGCAACAGCAACC
TGAGTCAGACCAGTAGGTCATCCCGGATGCTGGCAGTGTTTCCAGCGAATGGGAAGATGCACAGCACTGTGGATTGCA
ATGGTGTGGTTTCCTTGGTTGGTGGACCTTCAGTTCCTACATCGCCTGTTGGACAGCTTCTGCCAGAGGGAACAACCA
CTGAAACTGAAATGAGAAAGAGAAGGTCAAGTTCTTTCCACGTTTCCATGGACTTTCTAGAAGATCCTTCCCAAAGGC
AACGAGCAATGAGTATAGCCAGCATTCTAACAAATACAGTAGAAGAACTTGAAGAATCCAGGCAGAAATGCCCACCCT
GTTGGTATAAATTTTCCAACATATTCTTAATCTGGGACTGTTCTCCATATTGGTTAAAAGTGAAACATGTTGTCAACC
TGGTTGTGATGGACCCATTTGTTGACCTGGCCATCACCATCTGTATTGTCTTAAATACTCTTTTCATGGCCATGGAGC
ACTATCCAATGACGGACCATTTCAATAATGTGCTTACAGTAGGAAACTTGGTTTTCACTGGGATCTTTACAGCAGAAA
TGTTTCTGAAAATTATTGCCATGGATCCTTACTATTATTTCCAAGAAGGCTGGAATATCTTTGACGGTTTTATTGTGA
CGCTTAGCCTGGTAGAACTTGGACTCGCCAATGTGGAAGGATTATCTGTTCTCCGTTCATTTCGATTGCTGCGAGTTT
TCAAGTTGGCAAAATCTTGGCCAACGTTAAATATGCTAATAAAGATCATCGGCAATTCCGTGGGGGCTCTGGGAAATT
TAACCCTCGTCTTGGCCATCATCGTCTTCATTTTTGCCGTGGTCGGCATGCAGCTCTTTGGTAAAAGCTACAAAGATT
GTGTCTGCAAGATCGCCAGTGATTGTCAACTCCCACGCTGGCACATGAATGACTTCTTCCACTCCTTCCTGATTGTGT
TCCGCGTGCTGTGTGGGGAGTGGATAGAGACCATGTGGGACTGTATGGAGGTTGCTGGTCAAGCCATGTGCCTTACTG
TCTTCATGATGGTCATGGTGATTGGAAACCTAGTGGTCCTGAATCTCTTTCTGGCCTTGCTTCTGAGCTCATTTAGTG
CAGACAACCTTGCAGCCACTGATGATGATAATGAAATGAATAATCTCCAAATTGCTGTGGATAGGATGCACAAAGGAG
TAGCTTATGTGAAAAGAAAAATATATGAATTTATTCAACAGTCCTTCATTAGGAAACAAAAGATTTTAGATGAAATTA
AACCACTTGATGATCTAAACAACAAGAAAGACAGTTGTATGTCCAATCATACAGCAGAAATTGGGAAAGATCTTGACT
ATCTTAAAGATGTAAATGGAACTACAAGTGGTATAGGAACTGGCAGCAGTGTTGAAAAATACATTATTGATGAAAGTG
ATTACATGTCATTCATAAACAACCCCAGTCTTACTGTGACTGTACCAATTGCTGTAGGAGAATCTGACTTTGAAAATT
TAAACACGGAAGACTTTAGTAGTGAATCGGATCTGGAAGAAAGCAAAGAGAAACTGAATGAAAGCAGTAGCTCATCAG
AAGGTAGCACTGTGGACATCGGCGCACCTGTAGAAGAACAGCCCGTAGTGGAACCTGAAGAAACTCTTGAACCAGAAG
CTTGTTTCACTGAAGGCTGTGTACAAAGATTCAAGTGTTGTCAAATCAATGTGGAAGAAGGCAGAGGAAAACAATGGT
GGAACCTGAGAAGGACGTGTTTCCGAATAGTTGAACATAACTGGTTTGAGACCTTCATTGTTTTCATGATTCTCCTTA
GTAGTGGTGCTCTGGCATTTGAAGATATATATATTGATCAGCGAAAGACGATTAAGACGATGTTGGAATATGCTGACA
AGGTTTTCACTTACATTTTCATTCTGGAAATGCTTCTAAAATGGGTGGCATATGGCTATCAAACATATTTCACCAATG
CCTGGTGTTGGCTGGACTTCTTAATTGTTGATGTTTCATTGGTCAGTTTAACAGCAAATGCCTTGGGTTACTCAGAAC
TTGGAGCCATCAAATCTCTCAGGACACTAAGAGCTCTGAGACCTCTAAGAGCCTTATCTCGATTTGAAGGGATGAGGG
TGGTTGTGAATGCCCTTTTAGGAGCAATTCCATCCATCATGAATGTGCTTCTGGTTTGTCTTATATTCTGGCTAATTT
TCAGCATCATGGGCGTAAATTTGTTTGCTGGCAAATTCTACCACTGTATTAACACCACAACTGGTGACAGGTTTGACA
TCGAAGACGTGAATAATCATACTGATTGCCTAAAACTAATAGAAAGAAATGAGACTGCTCGATGGAAAAATGTGAAAG
TAAACTTTGATAATGTAGGATTTGGGTATCTCTCTTTGCTTCAAGTTGCCACATTCAAAGGATGGATGGATATAATGT
ATGCAGCAGTTGATTCCAGAAATGTGGAACTCCAGCCTAAGTATGAAGAAAGTCTGTACATGTATCTTTACTTTGTTA
TTTTCATCATCTTTGGGTCCTTCTTCACCTTGAACCTGTTTATTGGTGTCATCATAGATAATTTCAACCAGCAGAAAA
AGAAGTTTGGAGGTCAAGACATCTTTATGACAGAAGAACAGAAGAAATACTATAATGCAATGAAAAAATTAGGATCGA
AAAAACCGCAAAAGCCTATACCTCGACCAGGAAACAAATTTCAAGGAATGGTCTTTGACTTCGTAACCAGACAAGTTT
TTGACATAAGCATCATGATTCTCATCTGTCTTAACATGGTCACAATGATGGTGGAAACAGATGACCAGAGTGAATATG
TGACTACCATTTTGTCACGCATCAATCTGGTGTTCATTGTGCTATTTACTGGAGAGTGTGTACTGAAACTCATCTCTC
TACGCCATTATTATTTTACCATTGGATGGAATATTTTTGATTTTGTGGTTGTCATTCTCTCCATTGTAGGTATGTTTC
TTGCCGAGCTGATAGAAAAGTATTTCGTGTCCCCTACCCTGTTCCGAGTGATCCGTCTTGCTAGGATTGGCCGAATCC
TACGTCTGATCAAAGGAGCAAAGGGGATCCGCACGCTGCTCTTTGCTTTGATGATGTCCCTTCCTGCGTTGTTTAACA
TCGGCCTCCTACTCTTCCTAGTCATGTTCATCTACGCCATCTTTGGGATGTCCAACTTTGCCTATGTTAAGAGGGAAG
TTGGGATCGATGACATGTTCAACTTTGAGACCTTTGGCAACAGCATGATCTGCCTATTCCAAATTACAACCTCTGCTG
GCTGGGATGGATTGCTAGCACCCATTCTCAACAGTAAGCCACCCGACTGTGACCCTAATAAAGTTAACCCTGGAAGCT
CAGTTAAGGGAGACTGTGGGAACCCATCTGTTGGAATTTTCTTTTTTGTCAGTTACATCATCATATCCTTCCTGGTTG
TGGTGAACATGTACATCGCGGTCATCCTGGAGAACTTCAGTGTTGCTACTGAAGAAAGTGCAGAGCCTCTGAGTGAGG
ATGACTTTGAGATGTTCTATGAGGTTTGGGAGAAGTTTGATCCCGATGCAACTCAGTTCATGGAATTTGAAAAATTAT
CTCAGTTTGCAGCTGCGCTTGAACCGCCTCTCAATCTGCCACAACCAAACAAACTCCAGCTCATTGCCATGGATTTGC
CCATGGTGAGTGGTGACCGGATCCACTGTCTTGATATCTTATTTGCTTTTACAAAGCGGGTTCTAGGAGAGAGTGGAG
AGATGGATGCTCTACGAATACAGATGGAAGAGCGATTCATGGCTTCCAATCCTTCCAAGGTCTCCTATCAGCCAATCA
CTACTACTTTAAAACGAAAACAAGAGGAAGTATCTGCTGTCATTATTCAGCGTGCTTACAGACGCCACCTTTTAAAGC
GAACTGTAAAACAAGCTTCCTTTACGTACAATAAAAACAAAATCAAAGGTGGGGCTAATCTTCTTATAAAAGAAGACA
TGATAATTGACAGAATAAATGAAAACTCTATTACAGAAAAAACTGATCTGACCATGTCCACTGCAGCTTGTCCACCTT
CCTATGACCGGGTGACAAAGCCAATTGTGGAAAAACATGAGCAAGAAGGCAAAGATGAAAAAGCCAAAGGGAAATAAA
TGAAAATAAATAAAAATAATTGGGTGACAAATTGTTTACAGCCTGTGAAGGTGATGTATTTTTATCAACAGGACTCCT
TTAGGAGGTCAATGCCAAACTGACTGTTTTTACACAAATCTCCTTAAGGTCAGTGCCTACAATAAGACAGTGACCCCT
TGTCAGCAAACTGTGACTCTGTGTAAAGGGGAGATGACCTTGACAGGAGGTTACTGTTCTCACTACCAGCTGACACTG
CTGAAGATAAGATGCACAATGGCTAGTCAGACTGTAGGGACCAGTTTCAAGGGGTGCAAACCTGTGATTTTGGGGTTG
TTTAACATGAAACACTTTAGTGTAGTAATTGTATCCACTGTTTGCATTTCAACTGCCACATTTGTCACATTTTTATGG
AATCTGTTAGTGGATTCATCTTTTTGTTAATCCATGTGTTTATTATATGTGACTATTTTTGTAAACGAAGTTTCTGTT
GAGAAATAGGCTAAGGACCTCTATAACAGGTATGCCACCTGGGGGGTATGGCAACCACATGGCCCTCCCAGCTACACA
AAGTCGTGGTTTGCATGAGGGCATGCTGCACTTAGAGATCATGCATGAGAAAAAGTCACAAGAAAAACAAATTCTTAA
ATTTCACCATATTTCTGGGAGGGGTAATTGGGTGATAAGTGGAGGTGCTTTGTTGATCTTGTTTTGCGAAATCCAGCC
CCTAGACCAAGTAGATTATTTGTGGGTAGGCCAGTAAATCTTAGCAGGTGCAAACTTCATTCAAATGTTTGGAGTCAT
AAATGTTATGTTTCTTTTTGTTGTATTAAAAAAAAAACCTGAATAGTGAATATTGCCCCTCACCCTCCACCGCCAGAA
GACTGAATTGACCAAAATTACTCTTTATAAATTTCTGCTTTTTCCTGCACTTTGTTTAGCCATCTTCGGCTCTCAGCA
AGGTTGACACTGTATATGTTAATGAAATGCTATTTATTATGTAAATAGTCATTTTACCCTGTGGTGCACGTTTGAGCA
AACAAATAATGACCTAAGCACAGTATTTATTGCATCAAATATGTACCACAAGAAATGTAGAGTGCAAGCTTTACACAG
GTAATAAAATGTATTCTGTACCATTTATAGATAGTTTGGATGCTATCAATGCATGTTTATATTACCATGCTGCTGTAT
CTGGTTTCTCTCACTGCTCAGAATCTCATTTATGAGAAACCATATGTCAGTGGTAAAGTCAAGGAAATTGTTCAACAG
ATCTCATTTATTTAAGTCATTAAGCAATAGTTTGCAGCACTTTAACAGCTTTTTGGTTATTTTTACATTTTAAGTGGA
TAACATATGGTATATAGCCAGACTGTACAGACATGTTTAAAAAAACACACTGCTTAACCTATTAAATATGTGTTTAGA
ATTTTATAAGCAAATATAAATACTGTAAAAAGTCACTTTATTTTATTTTTCAGCATTATGTACATAAATATGAAGAGG
AAATTATCTTCAGGTTGATATCACAATCACTTTTCTTACTTTCTGTCCATAGTACTTTTTCATGAAAGAAATTTGCTA
AATAAGACATGAAAACAAGACTGGGTAGTTGTAGATTTCTGCTTTTTAAATTACATTTGCTAATTTTAGATTATTTCA
CAATTTTAAGGAGCAAAATAGGTTCACGATTCATATCCAAATTATGCTTTGCAATTGGAAAAGGGTTTAAAATTTTAT
TTATATTTCTGGTAGTACCTGCACTAACTGAATTGAAGGTAGTGCTTATGTTATTTTTGTTCTTTTTTTCTGACTTCG
GTTTATGTTTTCATTTCTTTGGAGTAATGCTGCTCTAGATTGTTCTAAATAGAATGTGGGCTTCATAATTTTTTTTTC
CACAAAAACAGAGTAGTCAACTTATATAGTCAATTACATCAGGACATTTTGTGTTTCTTACAGAAGCAAACCATAGGC
TCCTCTTTTCCTTAAAACTACTTAGATAAACTGTATTCGTGAACTGCATGCTGGAAAATGCTACTATTATGCTAAATA
ATGCTAACCAACATTTAAAATGTGCAAAACTAATAAAGATTACATTTTTTATTTTATTGTTTGCCCAGTCACTTTTTG
TTAACAGAATATTCTAATGATATGGAGATTTTTTACATTACAAATTGGGGGAGAAGGGGAGCGCGCGCGCACACACAC
ACACACACACACACACACACACACACACACAGAGGCATACCCACGTTGACAACAAAACCTAGGGTAGATATGTCACTG
GAGGTAGGGGGTAATGACCTCCCAGAATTACAAGCAGCAGGTGTGTTCTCTGTTAGGAGGAAGAACTGGTGTCAGAGG
ATAGCTAGTGATTCTAGGAGGAAGAGAAGTATGGAAGCCAGAGTGATGGTGGATGACCCCTTGAGCTATGAAAAGAAA
CCCTTAAATCATCATTTAAAAATTTAGAATTGCCATGTGTGTAGGATACTGTGTTTGCTCCTCCAGAGCCACTCTCTC
TGCTTCTGCATCATTCTGTGTGTCCCAGAAGGGTGACTTCTACACATTGCAAAAATGGGCTCTCCTACCTTTGAGCTC
CCAATTGGTTTGGCCAATGAGAAGCACCAGTGGGAAAGCACCAGAGAGAGAAGATTGACATAGGAATATTTCTTCTCC
AATTCCTTCTTTGCTGGGTTGGCACTGGACTCATTCCTCCCCGAAAAGTCATACTCCAATCAGACTGCCCCTCATACA
ACTGAAGCTACTTTCTCTGGGGTCAGGTAATCACTCCTCCCCTTGCTCCTTCAGGTCTGCTGCTGCATTGAGAGTGCT
TTTGTATTCCTTGTAGCTTTCTCCTAACATTGCTGACACTTTTGTAAATGTCCCCTTCATGAAATTCTTCTATATGCC
TCATTTCAGCATGCCATCTGTCTCCTGCCTGGCTGACACAAGGTGATTCAACAGCTCATGAAAGTCAGCAGGAAGCAA
AGATGTGCCTTGCTTCAGCTTGGGGTCTTAATCTTGCTAACTTTTGCAGATAAAGAAAAACAGTAACTGGGGGAACCA
CAGTGAAGTCCAGTGCAGAATTCACAGATATCATGGAAAGGTTACTCGGGTGGTCCAGATAGTAAAATTAACAGTCTA
AATTAATCTATCTAAATTTCTGAGGAACGAGAAGCCTTCCCTTGTCATCAGGTGAAGCCAGAAGAGGGAATATAGCCT
CAACCAGAAAAGGGACAGTAATTAAAAGGCTTTTCCCATCCTTGTACAATGGACTGACTTTGCCTCTTCATAACATCA
CAATCCTAAAGCAACACAACAATTAATTCTGATATATTAGTAGCTGAAAAAAATTCCCATTTCCAACTAAGGTAGGTC
AGAATTATAGGATAAACCCTGCAGACTTTTTATACTACCCATCCACGCCATTACTCACTGTTACCTTTCCAAATACAA
AGAGAAGAACTGGTAAAACATAATCATATAAATCTCCATATTCATTTTGAAATATTTGGCATGATATTTTCTGTGCTA
AAAAGTAATTATTCTTCAAAGAATGATGAGGTCATGTCAGTAAGACACAGGAACCAACTAGAAGGGGCTTCCCACTGG
CCAAATCTGGGGCAAGTTGAGCATCAAAATAAATGATAGTAAAAGATTATAATTCATTGAATAAGAATCAGCAAATAC
ATACTGATGTAAGTAAATAAGGAAAAGTACAAATCTGTTTCTTGCAGTTGAATGTTAATTAACAATTGTAGAAGAAAT
AACGGAGTTAGAAAAATCACTATTTGGCAATCACCCTAATGACAATTGATTCATACAAGAATCATCAATGAGTATTAA
AACTCATGGGTGAAAGTTTGATGAGGAATAGGGTATTTATAGCATCTTAAAGTATCTCTTCTCTATTAAGTAGAAAAT
TTAAACAGAAGAAAGTATACTTTGGAGAAATACAGCAGACAATACCTTCAAAGATATCATCAATTATGAGACCAACTG
ATACTATGTGCCTCCTGATAAGATATACTGAAAGGGCCACATTACTTCTTGGTACACAGTCAAAATTTTAAAACCAGA
ATCTAACTACAAGGAAAATCAAATTGAGGACACTCTATAAAATAAGTGGACTGAACTCCTTAAAAATGTCAATGTCAT
GAAAGACAAAGAAAGGCTAAAGAATTCCATGAGGTCAAAGAACTATGACAACTAAACACAATTCTGGATGGAATATCA
AATTAAAAAATAACAGATAAATAATATTATTGGGAAAGTTGAATAAATTTGAATATGGACTGTTTATTAGTTATTAGT
ATTATAATAGTGTTAATTTTCCTAATTTTGTTAAGACTAGTGTGCCTGTTCCATGAAAATAGAAAATGTTCTTATTCT
CTGAAAATGCATGCTAAAGTATTTAGGGGTGAATGCAACAATGTCTGCAGCTCATTCTTGAATCAGTTCAAAGAAAAA
TGAGTTACATTTATATATATATGTATGTATGTAAACAGACATAGATAAAAGTATAGATGTGTGTGTGTCTTTAGAAAG
GGGAGGATTTTTTTTTTTTTTTTGCTGTGTGTTACTGAAGTGCCTATGTCTGCGTGTTCACACTATCATATTTTGTAT
GCCCTGGACTTTATAATTTCTACCTTCAAAATTAGATCTACTGTTGGTAATTAATTCAATATATACTGGTTTTTTAAC
TACTATTCTCATTTCCTAGCAGTAATCTTCCTGAAAAGTCACAGAAATGATTACATTCCTTGTTCTTCATAATAATCA
CTGTTTAATTAAAATAAGAATATTTTAGAAAAGATCTGCGGCATAGTGGTTAAGACCCCAGTATTTGATGCTAAACAG
ATCTGATTTGGATAACAGAAGGTGGCACTTTGCTGTTTAAGCTGGGGACCAGACACTGTGGGTATAAATAGTAATTCC
AAACACAGCTCCACAGAGCAGCACCCTTATGACAAGGTTTTCATATGTCTATAGTTAAGCCAGAAAATTAAGAATAAT
GCCATAAATATTTATAAAGCTGAACATATCCAAGTTAAAGACCTTTATCCTGAAATTGTATCTTTTAGATTATTTTCT
AAAGACTAATACCATTTAATGTTTAAATGTTCTTTGGAAATGATGGTGAGAATACGTGATAATGGGTCATTGGTTTTA
ATATTTTATTTAGCCAAGTGGAAAATTGGCAACCTGGTGTCGGTCCTCCCATTTGTATTTTACTGGTGCATGAAATCC
AAAAGTCTAGTAACCATTGGGACAGACAACTCTACTGCATAAGTTTGTATGTTTGTATATCTGTATCACAAAGCCCAG
ACACTCGAACTATATAAACTTGTCGCACTAAAGACAGCAAATATGTCTGGTAATTGCATATTCTTCATGTGTGCACTG
GAATTTCTTATTATATAAGAAAATAAATGTGTTTCTAAACCACCATGAATTGGGTACTGCTGATAGTACTATTCTTCC
TGGCACATGGAAATATCCCACTGAGGTTGTCAATCAATATTGATTTAATCAGTGTGGCAGCAAAGGCACTAGAGGAGG
AAAATACTCTAGACAATCAAGTATAGATGGGAAAATTGCATCACCAAGAAATTCTTAAAATCACAAGTGGATAATTTG
CAATATCAATTGATTTTACTCAACTGCTTGAGTCCTTTATCATTCCGTTAGAACACTCCTCATATTTGCAGATAACTC
AGCCCCTCACCTCAGCAACAAGCAAGAGGACATCATTATAAACTTCCTCAATTTCTTTCCTATCCAGTGAAAAATATC
TAAGTCCCTTTTCCCTTCCCTTTTCCTTCCCTTCCCTTCCTTTTTCTTTCCTTCCATTCTCTTCCTTTTTCCTTCCTT
TTTCTTTCCTTCCATTCTCTTCCTTTTTCCTTCCTTTTTCTTACTTTACCTCCCCCTCCTACCTTTCCCTTTCTCCCT
TCCTCCCTCTCTCCCTTCCTTCCTCTCTCCCTCTCCTTCCTTCCTTCCTTCAATCCATACTTTTATATCTTTTCAGAG
ATCAATTTTTCCCATTCATTTCTACATTCTCAGATACCTTGCTCCATGACTTGTTCTACTTCTCTTCTATCTTTATTC
TTTCCCTCTTGACTGATCCTTTTTGTCTGCCTTTGCATTTTCAAAATCAGTTTTTCTAATCTTAAAAAATAACTTTGC
TCAACCCTGATGCATCTTTATTCACTGTGTCATTATCTCATTATTTTTAAAGGTTAGTTTCTAAAGCCAGGGCTCTAT
ACTCACTACCTTCACTCTTGGTTAAAATTAAAAGAAATATAGGTCCCAGTGATATTTATGGGAAAATTTGACTGTGAA
TAATCTTGTTGATATAAACACTTTGGGTTTTCAAAATGTTATGCTAGCTGTTAACTTTCCCAGTAACATTATGAGGGA
AGGGCTGGAAAATACAAAAGGTAGACATAGGAACCGAGAACACCACCCCTATGGAGCCAAGCTATACATACTCTGTCT
CTGGTTATCATAAATACATCTCCTATATTCAATGCTTGCTTAGAAAGGCATGATGTCCACGGAGCAATTCAATTGTTC
ACTGTGATGGGTTTTTTTGTTTTCTCCTCATGGAATGCAACTTTTATTTAT
SEQ ID NO: 5 >NM_001353958.2 SCN1A [organism = Homo sapiens] [GeneID = 6323]
[transcript = 13]
AAGTGTAGGAGACACACTGCTGGCCTGTGGAAACTCATGGAACTGTTCCTCCAGATTAACACTTCAGGGGTTATGGAA
GCTGGAGGAAGCTGAGCTTTTACTACATCTTTTGGGGGTTTGGGCAATTATGAATAAGGCTGCTGTATACATCCGTGT
GCAGGATTTTGTGTGGACATAAGTTTTCAACTCCTTTGGTTAAATCCTAAGGAATTTCATATGCAGAATAAATGGTAA
TTAAAATGTGCAGGATGACAAGATGGAGCAAACAGTGCTTGTACCACCAGGACCTGACAGCTTCAACTTCTTCACCAG
AGAATCTCTTGCGGCTATTGAAAGACGCATTGCAGAAGAAAAGGCAAAGAATCCCAAACCAGACAAAAAAGATGACGA
CGAAAATGGCCCAAAGCCAAATAGTGACTTGGAAGCTGGAAAGAACCTTCCATTTATTTATGGAGACATTCCTCCAGA
GATGGTGTCAGAGCCCCTGGAGGACCTGGACCCCTACTATATCAATAAGAAAACTTTTATAGTATTGAATAAAGGGAA
GGCCATCTTCCGGTTCAGTGCCACCTCTGCCCTGTACATTTTAACTCCCTTCAATCCTCTTAGGAAAATAGCTATTAA
GATTTTGGTACATTCATTATTCAGCATGCTAATTATGTGCACTATTTTGACAAACTGTGTGTTTATGACAATGAGTAA
CCCTCCTGATTGGACAAAGAATGTAGAATACACCTTCACAGGAATATATACTTTTGAATCACTTATAAAAATTATTGC
AAGGGGATTCTGTTTAGAAGATTTTACTTTCCTTCGGGATCCATGGAACTGGCTCGATTTCACTGTCATTACATTTGC
GTACGTCACAGAGTTTGTGGACCTGGGCAATGTCTCGGCATTGAGAACATTCAGAGTTCTCCGAGCATTGAAGACGAT
TTCAGTCATTCCAGGCCTGAAAACCATTGTGGGAGCCCTGATCCAGTCTGTGAAGAAGCTCTCAGATGTAATGATCCT
GACTGTGTTCTGTCTGAGCGTATTTGCTCTAATTGGGCTGCAGCTGTTCATGGGCAACCTGAGGAATAAATGTATACA
ATGGCCTCCCACCAATGCTTCCTTGGAGGAACATAGTATAGAAAAGAATATAACTGTGAATTATAATGGTACACTTAT
AAATGAAACTGTCTTTGAGTTTGACTGGAAGTCATATATTCAAGATTCAAGATATCATTATTTCCTGGAGGGTTTTTT
AGATGCACTACTATGTGGAAATAGCTCTGATGCAGGCCAATGTCCAGAGGGATATATGTGTGTGAAAGCTGGTAGAAA
TCCCAATTATGGCTACACAAGCTTTGATACCTTCAGTTGGGCTTTTTTGTCCTTGTTTCGACTAATGACTCAGGACTT
CTGGGAAAATCTTTATCAACTGACATTACGTGCTGCTGGGAAAACGTACATGATATTTTTTGTATTGGTCATTTTCTT
GGGCTCATTCTACCTAATAAATTTGATCCTGGCTGTGGTGGCCATGGCCTACGAGGAACAGAATCAGGCCACCTTGGA
AGAAGCAGAACAGAAAGAGGCCGAATTTCAGCAGATGATTGAACAGCTTAAAAAGCAACAGGAGGCAGCTCAGCAGGC
AGCAACGGCAACTGCCTCAGAACATTCCAGAGAGCCCAGTGCAGCAGGCAGGCTCTCAGACAGCTCATCTGAAGCCTC
TAAGTTGAGTTCCAAGAGTGCTAAGGAAAGAAGAAATCGGAGGAAGAAAAGAAAACAGAAAGAGCAGTCTGGTGGGGA
AGAGAAAGATGAGGATGAATTCCAAAAATCTGAATCTGAGGACAGCATCAGGAGGAAAGGTTTTCGCTTCTCCATTGA
AGGGAACCGATTGACATATGAAAAGAGGTACTCCTCCCCACACCAGTCTTTGTTGAGCATCCGTGGCTCCCTATTTTC
ACCAAGGCGAAATAGCAGAACAAGCCTTTTCAGCTTTAGAGGGCGAGCAAAGGATGTGGGATCTGAGAACGACTTCGC
AGATGATGAGCACAGCACCTTTGAGGATAACGAGAGCCGTAGAGATTCCTTGTTTGTGCCCCGACGACACGGAGAGAG
ACGCAACAGCAACCTGAGTCAGACCAGTAGGTCATCCCGGATGCTGGCAGTGTTTCCAGCGAATGGGAAGATGCACAG
CACTGTGGATTGCAATGGTGTGGTTTCCTTGGGAACAACCACTGAAACTGAAATGAGAAAGAGAAGGTCAAGTTCTTT
CCACGTTTCCATGGACTTTCTAGAAGATCCTTCCCAAAGGCAACGAGCAATGAGTATAGCCAGCATTCTAACAAATAC
AGTAGAAGAACTTGAAGAATCCAGGCAGAAATGCCCACCCTGTTGGTATAAATTTTCCAACATATTCTTAATCTGGGA
CTGTTCTCCATATTGGTTAAAAGTGAAACATGTTGTCAACCTGGTTGTGATGGACCCATTTGTTGACCTGGCCATCAC
CATCTGTATTGTCTTAAATACTCTTTTCATGGCCATGGAGCACTATCCAATGACGGACCATTTCAATAATGTGCTTAC
AGTAGGAAACTTGGTTTTCACTGGGATCTTTACAGCAGAAATGTTTCTGAAAATTATTGCCATGGATCCTTACTATTA
TTTCCAAGAAGGCTGGAATATCTTTGACGGTTTTATTGTGACGCTTAGCCTGGTAGAACTTGGACTCGCCAATGTGGA
AGGATTATCTGTTCTCCGTTCATTTCGATTGCTGCGAGTTTTCAAGTTGGCAAAATCTTGGCCAACGTTAAATATGCT
AATAAAGATCATCGGCAATTCCGTGGGGGCTCTGGGAAATTTAACCCTCGTCTTGGCCATCATCGTCTTCATTTTTGC
CGTGGTCGGCATGCAGCTCTTTGGTAAAAGCTACAAAGATTGTGTCTGCAAGATCGCCAGTGATTGTCAACTCCCACG
CTGGCACATGAATGACTTCTTCCACTCCTTCCTGATTGTGTTCCGCGTGCTGTGTGGGGAGTGGATAGAGACCATGTG
GGACTGTATGGAGGTTGCTGGTCAAGCCATGTGCCTTACTGTCTTCATGATGGTCATGGTGATTGGAAACCTAGTGGT
CCTGAATCTCTTTCTGGCCTTGCTTCTGAGCTCATTTAGTGCAGACAACCTTGCAGCCACTGATGATGATAATGAAAT
GAATAATCTCCAAATTGCTGTGGATAGGATGCACAAAGGAGTAGCTTATGTGAAAAGAAAAATATATGAATTTATTCA
ACAGTCCTTCATTAGGAAACAAAAGATTTTAGATGAAATTAAACCACTTGATGATCTAAACAACAAGAAAGACAGTTG
TATGTCCAATCATACAGCAGAAATTGGGAAAGATCTTGACTATCTTAAAGATGTAAATGGAACTACAAGTGGTATAGG
AACTGGCAGCAGTGTTGAAAAATACATTATTGATGAAAGTGATTACATGTCATTCATAAACAACCCCAGTCTTACTGT
GACTGTACCAATTGCTGTAGGAGAATCTGACTTTGAAAATTTAAACACGGAAGACTTTAGTAGTGAATCGGATCTGGA
AGAAAGCAAAGAGAAACTGAATGAAAGCAGTAGCTCATCAGAAGGTAGCACTGTGGACATCGGCGCACCTGTAGAAGA
ACAGCCCGTAGTGGAACCTGAAGAAACTCTTGAACCAGAAGCTTGTTTCACTGAAGGCTGTGTACAAAGATTCAAGTG
TTGTCAAATCAATGTGGAAGAAGGCAGAGGAAAACAATGGTGGAACCTGAGAAGGACGTGTTTCCGAATAGTTGAACA
TAACTGGTTTGAGACCTTCATTGTTTTCATGATTCTCCTTAGTAGTGGTGCTCTGGCATTTGAAGATATATATATTGA
TCAGCGAAAGACGATTAAGACGATGTTGGAATATGCTGACAAGGTTTTCACTTACATTTTCATTCTGGAAATGCTTCT
AAAATGGGTGGCATATGGCTATCAAACATATTTCACCAATGCCTGGTGTTGGCTGGACTTCTTAATTGTTGATGTTTC
ATTGGTCAGTTTAACAGCAAATGCCTTGGGTTACTCAGAACTTGGAGCCATCAAATCTCTCAGGACACTAAGAGCTCT
GAGACCTCTAAGAGCCTTATCTCGATTTGAAGGGATGAGGGTGGTTGTGAATGCCCTTTTAGGAGCAATTCCATCCAT
CATGAATGTGCTTCTGGTTTGTCTTATATTCTGGCTAATTTTCAGCATCATGGGCGTAAATTTGTTTGCTGGCAAATT
CTACCACTGTATTAACACCACAACTGGTGACAGGTTTGACATCGAAGACGTGAATAATCATACTGATTGCCTAAAACT
AATAGAAAGAAATGAGACTGCTCGATGGAAAAATGTGAAAGTAAACTTTGATAATGTAGGATTTGGGTATCTCTCTTT
GCTTCAAGTTGCCACATTCAAAGGATGGATGGATATAATGTATGCAGCAGTTGATTCCAGAAATGTGGAACTCCAGCC
TAAGTATGAAGAAAGTCTGTACATGTATCTTTACTTTGTTATTTTCATCATCTTTGGGTCCTTCTTCACCTTGAACCT
GTTTATTGGTGTCATCATAGATAATTTCAACCAGCAGAAAAAGAAGTTTGGAGGTCAAGACATCTTTATGACAGAAGA
ACAGAAGAAATACTATAATGCAATGAAAAAATTAGGATCGAAAAAACCGCAAAAGCCTATACCTCGACCAGGAAACAA
ATTTCAAGGAATGGTCTTTGACTTCGTAACCAGACAAGTTTTTGACATAAGCATCATGATTCTCATCTGTCTTAACAT
GGTCACAATGATGGTGGAAACAGATGACCAGAGTGAATATGTGACTACCATTTTGTCACGCATCAATCTGGTGTTCAT
TGTGCTATTTACTGGAGAGTGTGTACTGAAACTCATCTCTCTACGCCATTATTATTTTACCATTGGATGGAATATTTT
TGATTTTGTGGTTGTCATTCTCTCCATTGTAGGTATGTTTCTTGCCGAGCTGATAGAAAAGTATTTCGTGTCCCCTAC
CCTGTTCCGAGTGATCCGTCTTGCTAGGATTGGCCGAATCCTACGTCTGATCAAAGGAGCAAAGGGGATCCGCACGCT
GCTCTTTGCTTTGATGATGTCCCTTCCTGCGTTGTTTAACATCGGCCTCCTACTCTTCCTAGTCATGTTCATCTACGC
CATCTTTGGGATGTCCAACTTTGCCTATGTTAAGAGGGAAGTTGGGATCGATGACATGTTCAACTTTGAGACCTTTGG
CAACAGCATGATCTGCCTATTCCAAATTACAACCTCTGCTGGCTGGGATGGATTGCTAGCACCCATTCTCAACAGTAA
GCCACCCGACTGTGACCCTAATAAAGTTAACCCTGGAAGCTCAGTTAAGGGAGACTGTGGGAACCCATCTGTTGGAAT
TTTCTTTTTTGTCAGTTACATCATCATATCCTTCCTGGTTGTGGTGAACATGTACATCGCGGTCATCCTGGAGAACTT
CAGTGTTGCTACTGAAGAAAGTGCAGAGCCTCTGAGTGAGGATGACTTTGAGATGTTCTATGAGGTTTGGGAGAAGTT
TGATCCCGATGCAACTCAGTTCATGGAATTTGAAAAATTATCTCAGTTTGCAGCTGCGCTTGAACCGCCTCTCAATCT
GCCACAACCAAACAAACTCCAGCTCATTGCCATGGATTTGCCCATGGTGAGTGGTGACCGGATCCACTGTCTTGATAT
CTTATTTGCTTTTACAAAGCGGGTTCTAGGAGAGAGTGGAGAGATGGATGCTCTACGAATACAGATGGAAGAGCGATT
CATGGCTTCCAATCCTTCCAAGGTCTCCTATCAGCCAATCACTACTACTTTAAAACGAAAACAAGAGGAAGTATCTGC
TGTCATTATTCAGCGTGCTTACAGACGCCACCTTTTAAAGCGAACTGTAAAACAAGCTTCCTTTACGTACAATAAAAA
CAAAATCAAAGGTGGGGCTAATCTTCTTATAAAAGAAGACATGATAATTGACAGAATAAATGAAAACTCTATTACAGA
AAAAACTGATCTGACCATGTCCACTGCAGCTTGTCCACCTTCCTATGACCGGGTGACAAAGCCAATTGTGGAAAAACA
TGAGCAAGAAGGCAAAGATGAAAAAGCCAAAGGGAAATAAATGAAAATAAATAAAAATAATTGGGTGACAAATTGTTT
ACAGCCTGTGAAGGTGATGTATTTTTATCAACAGGACTCCTTTAGGAGGTCAATGCCAAACTGACTGTTTTTACACAA
ATCTCCTTAAGGTCAGTGCCTACAATAAGACAGTGACCCCTTGTCAGCAAACTGTGACTCTGTGTAAAGGGGAGATGA
CCTTGACAGGAGGTTACTGTTCTCACTACCAGCTGACACTGCTGAAGATAAGATGCACAATGGCTAGTCAGACTGTAG
GGACCAGTTTCAAGGGGTGCAAACCTGTGATTTTGGGGTTGTTTAACATGAAACACTTTAGTGTAGTAATTGTATCCA
CTGTTTGCATTTCAACTGCCACATTTGTCACATTTTTATGGAATCTGTTAGTGGATTCATCTTTTTGTTAATCCATGT
GTTTATTATATGTGACTATTTTTGTAAACGAAGTTTCTGTTGAGAAATAGGCTAAGGACCTCTATAACAGGTATGCCA
CCTGGGGGGTATGGCAACCACATGGCCCTCCCAGCTACACAAAGTCGTGGTTTGCATGAGGGCATGCTGCACTTAGAG
ATCATGCATGAGAAAAAGTCACAAGAAAAACAAATTCTTAAATTTCACCATATTTCTGGGAGGGGTAATTGGGTGATA
AGTGGAGGTGCTTTGTTGATCTTGTTTTGCGAAATCCAGCCCCTAGACCAAGTAGATTATTTGTGGGTAGGCCAGTAA
ATCTTAGCAGGTGCAAACTTCATTCAAATGTTTGGAGTCATAAATGTTATGTTTCTTTTTGTTGTATTAAAAAAAAAA
CCTGAATAGTGAATATTGCCCCTCACCCTCCACCGCCAGAAGACTGAATTGACCAAAATTACTCTTTATAAATTTCTG
CTTTTTCCTGCACTTTGTTTAGCCATCTTCGGCTCTCAGCAAGGTTGACACTGTATATGTTAATGAAATGCTATTTAT
TATGTAAATAGTCATTTTACCCTGTGGTGCACGTTTGAGCAAACAAATAATGACCTAAGCACAGTATTTATTGCATCA
AATATGTACCACAAGAAATGTAGAGTGCAAGCTTTACACAGGTAATAAAATGTATTCTGTACCATTTATAGATAGTTT
GGATGCTATCAATGCATGTTTATATTACCATGCTGCTGTATCTGGTTTCTCTCACTGCTCAGAATCTCATTTATGAGA
AACCATATGTCAGTGGTAAAGTCAAGGAAATTGTTCAACAGATCTCATTTATTTAAGTCATTAAGCAATAGTTTGCAG
CACTTTAACAGCTTTTTGGTTATTTTTACATTTTAAGTGGATAACATATGGTATATAGCCAGACTGTACAGACATGTT
TAAAAAAACACACTGCTTAACCTATTAAATATGTGTTTAGAATTTTATAAGCAAATATAAATACTGTAAAAAGTCACT
TTATTTTATTTTTCAGCATTATGTACATAAATATGAAGAGGAAATTATCTTCAGGTTGATATCACAATCACTTTTCTT
ACTTTCTGTCCATAGTACTTTTTCATGAAAGAAATTTGCTAAATAAGACATGAAAACAAGACTGGGTAGTTGTAGATT
TCTGCTTTTTAAATTACATTTGCTAATTTTAGATTATTTCACAATTTTAAGGAGCAAAATAGGTTCACGATTCATATC
CAAATTATGCTTTGCAATTGGAAAAGGGTTTAAAATTTTATTTATATTTCTGGTAGTACCTGCACTAACTGAATTGAA
GGTAGTGCTTATGTTATTTTTGTTCTTTTTTTCTGACTTCGGTTTATGTTTTCATTTCTTTGGAGTAATGCTGCTCTA
GATTGTTCTAAATAGAATGTGGGCTTCATAATTTTTTTTTCCACAAAAACAGAGTAGTCAACTTATATAGTCAATTAC
ATCAGGACATTTTGTGTTTCTTACAGAAGCAAACCATAGGCTCCTCTTTTCCTTAAAACTACTTAGATAAACTGTATT
CGTGAACTGCATGCTGGAAAATGCTACTATTATGCTAAATAATGCTAACCAACATTTAAAATGTGCAAAACTAATAAA
GATTACATTTTTTATTTTATTGTTTGCCCAGTCACTTTTTGTTAACAGAATATTCTAATGATATGGAGATTTTTTACA
TTACAAATTGGGGGAGAAGGGGAGCGCGCGCGCACACACACACACACACACACACACACACACACACACACAGAGGCA
TACCCACGTTGACAACAAAACCTAGGGTAGATATGTCACTGGAGGTAGGGGGTAATGACCTCCCAGAATTACAAGCAG
CAGGTGTGTTCTCTGTTAGGAGGAAGAACTGGTGTCAGAGGATAGCTAGTGATTCTAGGAGGAAGAGAAGTATGGAAG
CCAGAGTGATGGTGGATGACCCCTTGAGCTATGAAAAGAAACCCTTAAATCATCATTTAAAAATTTAGAATTGCCATG
TGTGTAGGATACTGTGTTTGCTCCTCCAGAGCCACTCTCTCTGCTTCTGCATCATTCTGTGTGTCCCAGAAGGGTGAC
TTCTACACATTGCAAAAATGGGCTCTCCTACCTTTGAGCTCCCAATTGGTTTGGCCAATGAGAAGCACCAGTGGGAAA
GCACCAGAGAGAGAAGATTGACATAGGAATATTTCTTCTCCAATTCCTTCTTTGCTGGGTTGGCACTGGACTCATTCC
TCCCCGAAAAGTCATACTCCAATCAGACTGCCCCTCATACAACTGAAGCTACTTTCTCTGGGGTCAGGTAATCACTCC
TCCCCTTGCTCCTTCAGGTCTGCTGCTGCATTGAGAGTGCTTTTGTATTCCTTGTAGCTTTCTCCTAACATTGCTGAC
ACTTTTGTAAATGTCCCCTTCATGAAATTCTTCTATATGCCTCATTTCAGCATGCCATCTGTCTCCTGCCTGGCTGAC
ACAAGGTGATTCAACAGCTCATGAAAGTCAGCAGGAAGCAAAGATGTGCCTTGCTTCAGCTTGGGGTCTTAATCTTGC
TAACTTTTGCAGATAAAGAAAAACAGTAACTGGGGGAACCACAGTGAAGTCCAGTGCAGAATTCACAGATATCATGGA
AAGGTTACTCGGGTGGTCCAGATAGTAAAATTAACAGTCTAAATTAATCTATCTAAATTTCTGAGGAACGAGAAGCCT
TCCCTTGTCATCAGGTGAAGCCAGAAGAGGGAATATAGCCTCAACCAGAAAAGGGACAGTAATTAAAAGGCTTTTCCC
ATCCTTGTACAATGGACTGACTTTGCCTCTTCATAACATCACAATCCTAAAGCAACACAACAATTAATTCTGATATAT
TAGTAGCTGAAAAAAATTCCCATTTCCAACTAAGGTAGGTCAGAATTATAGGATAAACCCTGCAGACTTTTTATACTA
CCCATCCACGCCATTACTCACTGTTACCTTTCCAAATACAAAGAGAAGAACTGGTAAAACATAATCATATAAATCTCC
ATATTCATTTTGAAATATTTGGCATGATATTTTCTGTGCTAAAAAGTAATTATTCTTCAAAGAATGATGAGGTCATGT
CAGTAAGACACAGGAACCAACTAGAAGGGGCTTCCCACTGGCCAAATCTGGGGCAAGTTGAGCATCAAAATAAATGAT
AGTAAAAGATTATAATTCATTGAATAAGAATCAGCAAATACATACTGATGTAAGTAAATAAGGAAAAGTACAAATCTG
TTTCTTGCAGTTGAATGTTAATTAACAATTGTAGAAGAAATAACGGAGTTAGAAAAATCACTATTTGGCAATCACCCT
AATGACAATTGATTCATACAAGAATCATCAATGAGTATTAAAACTCATGGGTGAAAGTTTGATGAGGAATAGGGTATT
TATAGCATCTTAAAGTATCTCTTCTCTATTAAGTAGAAAATTTAAACAGAAGAAAGTATACTTTGGAGAAATACAGCA
GACAATACCTTCAAAGATATCATCAATTATGAGACCAACTGATACTATGTGCCTCCTGATAAGATATACTGAAAGGGC
CACATTACTTCTTGGTACACAGTCAAAATTTTAAAACCAGAATCTAACTACAAGGAAAATCAAATTGAGGACACTCTA
TAAAATAAGTGGACTGAACTCCTTAAAAATGTCAATGTCATGAAAGACAAAGAAAGGCTAAAGAATTCCATGAGGTCA
AAGAACTATGACAACTAAACACAATTCTGGATGGAATATCAAATTAAAAAATAACAGATAAATAATATTATTGGGAAA
GTTGAATAAATTTGAATATGGACTGTTTATTAGTTATTAGTATTATAATAGTGTTAATTTTCCTAATTTTGTTAAGAC
TAGTGTGCCTGTTCCATGAAAATAGAAAATGTTCTTATTCTCTGAAAATGCATGCTAAAGTATTTAGGGGTGAATGCA
ACAATGTCTGCAGCTCATTCTTGAATCAGTTCAAAGAAAAATGAGTTACATTTATATATATATGTATGTATGTAAACA
GACATAGATAAAAGTATAGATGTGTGTGTGTCTTTAGAAAGGGGAGGATTTTTTTTTTTTTTTTGCTGTGTGTTACTG
AAGTGCCTATGTCTGCGTGTTCACACTATCATATTTTGTATGCCCTGGACTTTATAATTTCTACCTTCAAAATTAGAT
CTACTGTTGGTAATTAATTCAATATATACTGGTTTTTTAACTACTATTCTCATTTCCTAGCAGTAATCTTCCTGAAAA
GTCACAGAAATGATTACATTCCTTGTTCTTCATAATAATCACTGTTTAATTAAAATAAGAATATTTTAGAAAAGATCT
GCGGCATAGTGGTTAAGACCCCAGTATTTGATGCTAAACAGATCTGATTTGGATAACAGAAGGTGGCACTTTGCTGTT
TAAGCTGGGGACCAGACACTGTGGGTATAAATAGTAATTCCAAACACAGCTCCACAGAGCAGCACCCTTATGACAAGG
TTTTCATATGTCTATAGTTAAGCCAGAAAATTAAGAATAATGCCATAAATATTTATAAAGCTGAACATATCCAAGTTA
AAGACCTTTATCCTGAAATTGTATCTTTTAGATTATTTTCTAAAGACTAATACCATTTAATGTTTAAATGTTCTTTGG
AAATGATGGTGAGAATACGTGATAATGGGTCATTGGTTTTAATATTTTATTTAGCCAAGTGGAAAATTGGCAACCTGG
TGTCGGTCCTCCCATTTGTATTTTACTGGTGCATGAAATCCAAAAGTCTAGTAACCATTGGGACAGACAACTCTACTG
CATAAGTTTGTATGTTTGTATATCTGTATCACAAAGCCCAGACACTCGAACTATATAAACTTGTCGCACTAAAGACAG
CAAATATGTCTGGTAATTGCATATTCTTCATGTGTGCACTGGAATTTCTTATTATATAAGAAAATAAATGTGTTTCTA
AACCACCATGAATTGGGTACTGCTGATAGTACTATTCTTCCTGGCACATGGAAATATCCCACTGAGGTTGTCAATCAA
TATTGATTTAATCAGTGTGGCAGCAAAGGCACTAGAGGAGGAAAATACTCTAGACAATCAAGTATAGATGGGAAAATT
GCATCACCAAGAAATTCTTAAAATCACAAGTGGATAATTTGCAATATCAATTGATTTTACTCAACTGCTTGAGTCCTT
TATCATTCCGTTAGAACACTCCTCATATTTGCAGATAACTCAGCCCCTCACCTCAGCAACAAGCAAGAGGACATCATT
ATAAACTTCCTCAATTTCTTTCCTATCCAGTGAAAAATATCTAAGTCCCTTTTCCCTTCCCTTTTCCTTCCCTTCCCT
TCCTTTTTCTTTCCTTCCATTCTCTTCCTTTTTCCTTCCTTTTTCTTTCCTTCCATTCTCTTCCTTTTTCCTTCCTTT
TTCTTACTTTACCTCCCCCTCCTACCTTTCCCTTTCTCCCTTCCTCCCTCTCTCCCTTCCTTCCTCTCTCCCTCTCCT
TCCTTCCTTCCTTCAATCCATACTTTTATATCTTTTCAGAGATCAATTTTTCCCATTCATTTCTACATTCTCAGATAC
CTTGCTCCATGACTTGTTCTACTTCTCTTCTATCTTTATTCTTTCCCTCTTGACTGATCCTTTTTGTCTGCCTTTGCA
TTTTCAAAATCAGTTTTTCTAATCTTAAAAAATAACTTTGCTCAACCCTGATGCATCTTTATTCACTGTGTCATTATC
TCATTATTTTTAAAGGTTAGTTTCTAAAGCCAGGGCTCTATACTCACTACCTTCACTCTTGGTTAAAATTAAAAGAAA
TATAGGTCCCAGTGATATTTATGGGAAAATTTGACTGTGAATAATCTTGTTGATATAAACACTTTGGGTTTTCAAAAT
GTTATGCTAGCTGTTAACTTTCCCAGTAACATTATGAGGGAAGGGCTGGAAAATACAAAAGGTAGACATAGGAACCGA
GAACACCACCCCTATGGAGCCAAGCTATACATACTCTGTCTCTGGTTATCATAAATACATCTCCTATATTCAATGCTT
GCTTAGAAAGGCATGATGTCCACGGAGCAATTCAATTGTTCACTGTGATGGGTTTTTTTGTTTTCTCCTCATGGAATG
CAACTTTTATTTAT
SEQ ID NO: 6 >NM_001353950.2 SCN1A [organism = Homo sapiens] [GeneID = 6323]
[transcript = 7]
AAGTGTAGGAGACACACTGCTGGCCTGTGGAAACTCATGGAACTGTTCCTCCAGATTAACACTTCAGGGGTTATGGAA
GCTGGAGGAAGCTGAGCTTTTACTACATCTTTTGGGGGTTTGGGCAATTATGAATAAGGCTGCTGTATACATCCGTGT
GCAGGATTTTGTGTGGACATAAGTTTTCAACTCCTTTGGTTAAATCCTAAGGAATTTCATATGCAGAATAAATGGTAA
TTAAAATGTGCAGGATGACAAGATGGAGCAAACAGTGCTTGTACCACCAGGACCTGACAGCTTCAACTTCTTCACCAG
AGAATCTCTTGCGGCTATTGAAAGACGCATTGCAGAAGAAAAGGCAAAGAATCCCAAACCAGACAAAAAAGATGACGA
CGAAAATGGCCCAAAGCCAAATAGTGACTTGGAAGCTGGAAAGAACCTTCCATTTATTTATGGAGACATTCCTCCAGA
GATGGTGTCAGAGCCCCTGGAGGACCTGGACCCCTACTATATCAATAAGAAAACTTTTATAGTATTGAATAAAGGGAA
GGCCATCTTCCGGTTCAGTGCCACCTCTGCCCTGTACATTTTAACTCCCTTCAATCCTCTTAGGAAAATAGCTATTAA
GATTTTGGTACATTCATTATTCAGCATGCTAATTATGTGCACTATTTTGACAAACTGTGTGTTTATGACAATGAGTAA
CCCTCCTGATTGGACAAAGAATGTAGAATACACCTTCACAGGAATATATACTTTTGAATCACTTATAAAAATTATTGC
AAGGGGATTCTGTTTAGAAGATTTTACTTTCCTTCGGGATCCATGGAACTGGCTCGATTTCACTGTCATTACATTTGC
GTACGTCACAGAGTTTGTGGACCTGGGCAATGTCTCGGCATTGAGAACATTCAGAGTTCTCCGAGCATTGAAGACGAT
TTCAGTCATTCCAGGCCTGAAAACCATTGTGGGAGCCCTGATCCAGTCTGTGAAGAAGCTCTCAGATGTAATGATCCT
GACTGTGTTCTGTCTGAGCGTATTTGCTCTAATTGGGCTGCAGCTGTTCATGGGCAACCTGAGGAATAAATGTATACA
ATGGCCTCCCACCAATGCTTCCTTGGAGGAACATAGTATAGAAAAGAATATAACTGTGAATTATAATGGTACACTTAT
AAATGAAACTGTCTTTGAGTTTGACTGGAAGTCATATATTCAAGATTCAAGATATCATTATTTCCTGGAGGGTTTTTT
AGATGCACTACTATGTGGAAATAGCTCTGATGCAGGCCAATGTCCAGAGGGATATATGTGTGTGAAAGCTGGTAGAAA
TCCCAATTATGGCTACACAAGCTTTGATACCTTCAGTTGGGCTTTTTTGTCCTTGTTTCGACTAATGACTCAGGACTT
CTGGGAAAATCTTTATCAACTGACATTACGTGCTGCTGGGAAAACGTACATGATATTTTTTGTATTGGTCATTTTCTT
GGGCTCATTCTACCTAATAAATTTGATCCTGGCTGTGGTGGCCATGGCCTACGAGGAACAGAATCAGGCCACCTTGGA
AGAAGCAGAACAGAAAGAGGCCGAATTTCAGCAGATGATTGAACAGCTTAAAAAGCAACAGGAGGCAGCTCAGCAGGC
AGCAACGGCAACTGCCTCAGAACATTCCAGAGAGCCCAGTGCAGCAGGCAGGCTCTCAGACAGCTCATCTGAAGCCTC
TAAGTTGAGTTCCAAGAGTGCTAAGGAAAGAAGAAATCGGAGGAAGAAAAGAAAACAGAAAGAGCAGTCTGGTGGGGA
AGAGAAAGATGAGGATGAATTCCAAAAATCTGAATCTGAGGACAGCATCAGGAGGAAAGGTTTTCGCTTCTCCATTGA
AGGGAACCGATTGACATATGAAAAGAGGTACTCCTCCCCACACCAGTCTTTGTTGAGCATCCGTGGCTCCCTATTTTC
ACCAAGGCGAAATAGCAGAACAAGCCTTTTCAGCTTTAGAGGGCGAGCAAAGGATGTGGGATCTGAGAACGACTTCGC
AGATGATGAGCACAGCACCTTTGAGGATAACGAGAGCCGTAGAGATTCCTTGTTTGTGCCCCGACGACACGGAGAGAG
ACGCAACAGCAACCTGAGTCAGACCAGTAGGTCATCCCGGATGCTGGCAGTGTTTCCAGCGAATGGGAAGATGCACAG
CACTGTGGATTGCAATGGTGTGGTTTCCTTGGTTGGTGGACCTTCAGTTCCTACATCGCCTGTTGGACAGCTTCTGCC
AGAGGGAACAACCACTGAAACTGAAATGAGAAAGAGAAGGTCAAGTTCTTTCCACGTTTCCATGGACTTTCTAGAAGA
TCCTTCCCAAAGGCAACGAGCAATGAGTATAGCCAGCATTCTAACAAATACAGTAGAAGAACTTGAAGAATCCAGGCA
GAAATGCCCACCCTGTTGGTATAAATTTTCCAACATATTCTTAATCTGGGACTGTTCTCCATATTGGTTAAAAGTGAA
ACATGTTGTCAACCTGGTTGTGATGGACCCATTTGTTGACCTGGCCATCACCATCTGTATTGTCTTAAATACTCTTTT
CATGGCCATGGAGCACTATCCAATGACGGACCATTTCAATAATGTGCTTACAGTAGGAAACTTGGTTTTCACTGGGAT
CTTTACAGCAGAAATGTTTCTGAAAATTATTGCCATGGATCCTTACTATTATTTCCAAGAAGGCTGGAATATCTTTGA
CGGTTTTATTGTGACGCTTAGCCTGGTAGAACTTGGACTCGCCAATGTGGAAGGATTATCTGTTCTCCGTTCATTTCG
ATTGCTGCGAGTTTTCAAGTTGGCAAAATCTTGGCCAACGTTAAATATGCTAATAAAGATCATCGGCAATTCCGTGGG
GGCTCTGGGAAATTTAACCCTCGTCTTGGCCATCATCGTCTTCATTTTTGCCGTGGTCGGCATGCAGCTCTTTGGTAA
AAGCTACAAAGATTGTGTCTGCAAGATCGCCAGTGATTGTCAACTCCCACGCTGGCACATGAATGACTTCTTCCACTC
CTTCCTGATTGTGTTCCGCGTGCTGTGTGGGGAGTGGATAGAGACCATGTGGGACTGTATGGAGGTTGCTGGTCAAGC
CATGTGCCTTACTGTCTTCATGATGGTCATGGTGATTGGAAACCTAGTGGTCCTGAATCTCTTTCTGGCCTTGCTTCT
GAGCTCATTTAGTGCAGACAACCTTGCAGCCACTGATGATGATAATGAAATGAATAATCTCCAAATTGCTGTGGATAG
GATGCACAAAGGAGTAGCTTATGTGAAAAGAAAAATATATGAATTTATTCAACAGTCCTTCATTAGGAAACAAAAGAT
TTTAGATGAAATTAAACCACTTGATGATCTAAACAACAAGAAAGACAGTTGTATGTCCAATCATACAGCAGAAATTGG
GAAAGATCTTGACTATCTTAAAGATGTAAATGGAACTACAAGTGGTATAGGAACTGGCAGCAGTGTTGAAAAATACAT
TATTGATGAAAGTGATTACATGTCATTCATAAACAACCCCAGTCTTACTGTGACTGTACCAATTGCTGTAGGAGAATC
TGACTTTGAAAATTTAAACACGGAAGACTTTAGTAGTGAATCGGATCTGGAAGAAAGCAAAGAGAAACTGAATGAAAG
CAGTAGCTCATCAGAAGGTAGCACTGTGGACATCGGCGCACCTGTAGAAGAACAGCCCGTAGTGGAACCTGAAGAAAC
TCTTGAACCAGAAGCTTGTTTCACTGAAGGCTGTGTACAAAGATTCAAGTGTTGTCAAATCAATGTGGAAGAAGGCAG
AGGAAAACAATGGTGGAACCTGAGAAGGACGTGTTTCCGAATAGTTGAACATAACTGGTTTGAGACCTTCATTGTTTT
CATGATTCTCCTTAGTAGTGGTGCTCTGGCATTTGAAGATATATATATTGATCAGCGAAAGACGATTAAGACGATGTT
GGAATATGCTGACAAGGTTTTCACTTACATTTTCATTCTGGAAATGCTTCTAAAATGGGTGGCATATGGCTATCAAAC
ATATTTCACCAATGCCTGGTGTTGGCTGGACTTCTTAATTGTTGATGTTTCATTGGTCAGTTTAACAGCAAATGCCTT
GGGTTACTCAGAACTTGGAGCCATCAAATCTCTCAGGACACTAAGAGCTCTGAGACCTCTAAGAGCCTTATCTCGATT
TGAAGGGATGAGGGTGGTTGTGAATGCCCTTTTAGGAGCAATTCCATCCATCATGAATGTGCTTCTGGTTTGTCTTAT
ATTCTGGCTAATTTTCAGCATCATGGGCGTAAATTTGTTTGCTGGCAAATTCTACCACTGTATTAACACCACAACTGG
TGACAGGTTTGACATCGAAGACGTGAATAATCATACTGATTGCCTAAAACTAATAGAAAGAAATGAGACTGCTCGATG
GAAAAATGTGAAAGTAAACTTTGATAATGTAGGATTTGGGTATCTCTCTTTGCTTCAAGTTGCCACATTCAAAGGATG
GATGGATATAATGTATGCAGCAGTTGATTCCAGAAATGTGGAACTCCAGCCTAAGTATGAAGAAAGTCTGTACATGTA
TCTTTACTTTGTTATTTTCATCATCTTTGGGTCCTTCTTCACCTTGAACCTGTTTATTGGTGTCATCATAGATAATTT
CAACCAGCAGAAAAAGAAGTTTGGAGGTCAAGACATCTTTATGACAGAAGAACAGAAGAAATACTATAATGCAATGAA
AAAATTAGGATCGAAAAAACCGCAAAAGCCTATACCTCGACCAGGAAACAAATTTCAAGGAATGGTCTTTGACTTCGT
AACCAGACAAGTTTTTGACATAAGCATCATGATTCTCATCTGTCTTAACATGGTCACAATGATGGTGGAAACAGATGA
CCAGAGTGAATATGTGACTACCATTTTGTCACGCATCAATCTGGTGTTCATTGTGCTATTTACTGGAGAGTGTGTACT
GAAACTCATCTCTCTACGCCATTATTATTTTACCATTGGATGGAATATTTTTGATTTTGTGGTTGTCATTCTCTCCAT
TGTAGGTATGTTTCTTGCCGAGCTGATAGAAAAGTATTTCGTGTCCCCTACCCTGTTCCGAGTGATCCGTCTTGCTAG
GATTGGCCGAATCCTACGTCTGATCAAAGGAGCAAAGGGGATCCGCACGCTGCTCTTTGCTTTGATGATGTCCCTTCC
TGCGTTGTTTAACATCGGCCTCCTACTCTTCCTAGTCATGTTCATCTACGCCATCTTTGGGATGTCCAACTTTGCCTA
TGTTAAGAGGGAAGTTGGGATCGATGACATGTTCAACTTTGAGACCTTTGGCAACAGCATGATCTGCCTATTCCAAAT
TACAACCTCTGCTGGCTGGGATGGATTGCTAGCACCCATTCTCAACAGTAAGCCACCCGACTGTGACCCTAATAAAGT
TAACCCTGGAAGCTCAGTTAAGGGAGACTGTGGGAACCCATCTGTTGGAATTTTCTTTTTTGTCAGTTACATCATCAT
ATCCTTCCTGGTTGTGGTGAACATGTACATCGCGGTCATCCTGGAGAACTTCAGTGTTGCTACTGAAGAAAGTGCAGA
GCCTCTGAGTGAGGATGACTTTGAGATGTTCTATGAGGTTTGGGAGAAGTTTGATCCCGATGCAACTCAGTTCATGGA
ATTTGAAAAATTATCTCAGTTTGCAGCTGCGCTTGAACCGCCTCTCAATCTGCCACAACCAAACAAACTCCAGCTCAT
TGCCATGGATTTGCCCATGGTGAGTGGTGACCGGATCCACTGTCTTGATATCTTATTTGCTTTTACAAAGCGGGTTCT
AGGAGAGAGTGGAGAGATGGATGCTCTACGAATACAGATGGAAGAGCGATTCATGGCTTCCAATCCTTCCAAGGTCTC
CTATCAGCCAATCACTACTACTTTAAAACGAAAACAAGAGGAAGTATCTGCTGTCATTATTCAGCGTGCTTACAGACG
CCACCTTTTAAAGCGAACTGTAAAACAAGCTTCCTTTACGTACAATAAAAACAAAATCAAAGGTGGGGCTAATCTTCT
TATAAAAGAAGACATGATAATTGACAGAATAAATGAAAACTCTATTACAGAAAAAACTGATCTGACCATGTCCACTGC
AGCTTGTCCACCTTCCTATGACCGGGTGACAAAGCCAATTGTGGAAAAACATGAGCAAGAAGGCAAAGATGAAAAAGC
CAAAGGGAAATAAATGAAAATAAATAAAAATAATTGGGTGACAAATTGTTTACAGCCTGTGAAGGTGATGTATTTTTA
TCAACAGGACTCCTTTAGGAGGTCAATGCCAAACTGACTGTTTTTACACAAATCTCCTTAAGGTCAGTGCCTACAATA
AGACAGTGACCCCTTGTCAGCAAACTGTGACTCTGTGTAAAGGGGAGATGACCTTGACAGGAGGTTACTGTTCTCACT
ACCAGCTGACACTGCTGAAGATAAGATGCACAATGGCTAGTCAGACTGTAGGGACCAGTTTCAAGGGGTGCAAACCTG
TGATTTTGGGGTTGTTTAACATGAAACACTTTAGTGTAGTAATTGTATCCACTGTTTGCATTTCAACTGCCACATTTG
TCACATTTTTATGGAATCTGTTAGTGGATTCATCTTTTTGTTAATCCATGTGTTTATTATATGTGACTATTTTTGTAA
ACGAAGTTTCTGTTGAGAAATAGGCTAAGGACCTCTATAACAGGTATGCCACCTGGGGGGTATGGCAACCACATGGCC
CTCCCAGCTACACAAAGTCGTGGTTTGCATGAGGGCATGCTGCACTTAGAGATCATGCATGAGAAAAAGTCACAAGAA
AAACAAATTCTTAAATTTCACCATATTTCTGGGAGGGGTAATTGGGTGATAAGTGGAGGTGCTTTGTTGATCTTGTTT
TGCGAAATCCAGCCCCTAGACCAAGTAGATTATTTGTGGGTAGGCCAGTAAATCTTAGCAGGTGCAAACTTCATTCAA
ATGTTTGGAGTCATAAATGTTATGTTTCTTTTTGTTGTATTAAAAAAAAAACCTGAATAGTGAATATTGCCCCTCACC
CTCCACCGCCAGAAGACTGAATTGACCAAAATTACTCTTTATAAATTTCTGCTTTTTCCTGCACTTTGTTTAGCCATC
TTCGGCTCTCAGCAAGGTTGACACTGTATATGTTAATGAAATGCTATTTATTATGTAAATAGTCATTTTACCCTGTGG
TGCACGTTTGAGCAAACAAATAATGACCTAAGCACAGTATTTATTGCATCAAATATGTACCACAAGAAATGTAGAGTG
CAAGCTTTACACAGGTAATAAAATGTATTCTGTACCATTTATAGATAGTTTGGATGCTATCAATGCATGTTTATATTA
CCATGCTGCTGTATCTGGTTTCTCTCACTGCTCAGAATCTCATTTATGAGAAACCATATGTCAGTGGTAAAGTCAAGG
AAATTGTTCAACAGATCTCATTTATTTAAGTCATTAAGCAATAGTTTGCAGCACTTTAACAGCTTTTTGGTTATTTTT
ACATTTTAAGTGGATAACATATGGTATATAGCCAGACTGTACAGACATGTTTAAAAAAACACACTGCTTAACCTATTA
AATATGTGTTTAGAATTTTATAAGCAAATATAAATACTGTAAAAAGTCACTTTATTTTATTTTTCAGCATTATGTACA
TAAATATGAAGAGGAAATTATCTTCAGGTTGATATCACAATCACTTTTCTTACTTTCTGTCCATAGTACTTTTTCATG
AAAGAAATTTGCTAAATAAGACATGAAAACAAGACTGGGTAGTTGTAGATTTCTGCTTTTTAAATTACATTTGCTAAT
TTTAGATTATTTCACAATTTTAAGGAGCAAAATAGGTTCACGATTCATATCCAAATTATGCTTTGCAATTGGAAAAGG
GTTTAAAATTTTATTTATATTTCTGGTAGTACCTGCACTAACTGAATTGAAGGTAGTGCTTATGTTATTTTTGTTCTT
TTTTTCTGACTTCGGTTTATGTTTTCATTTCTTTGGAGTAATGCTGCTCTAGATTGTTCTAAATAGAATGTGGGCTTC
ATAATTTTTTTTTCCACAAAAACAGAGTAGTCAACTTATATAGTCAATTACATCAGGACATTTTGTGTTTCTTACAGA
AGCAAACCATAGGCTCCTCTTTTCCTTAAAACTACTTAGATAAACTGTATTCGTGAACTGCATGCTGGAAAATGCTAC
TATTATGCTAAATAATGCTAACCAACATTTAAAATGTGCAAAACTAATAAAGATTACATTTTTTATTTTATTGTTTGC
CCAGTCACTTTTTGTTAACAGAATATTCTAATGATATGGAGATTTTTTACATTACAAATTGGGGGAGAAGGGGAGCGC
GCGCGCACACACACACACACACACACACACACACACACACACACAGAGGCATACCCACGTTGACAACAAAACCTAGGG
TAGATATGTCACTGGAGGTAGGGGGTAATGACCTCCCAGAATTACAAGCAGCAGGTGTGTTCTCTGTTAGGAGGAAGA
ACTGGTGTCAGAGGATAGCTAGTGATTCTAGGAGGAAGAGAAGTATGGAAGCCAGAGTGATGGTGGATGACCCCTTGA
GCTATGAAAAGAAACCCTTAAATCATCATTTAAAAATTTAGAATTGCCATGTGTGTAGGATACTGTGTTTGCTCCTCC
AGAGCCACTCTCTCTGCTTCTGCATCATTCTGTGTGTCCCAGAAGGGTGACTTCTACACATTGCAAAAATGGGCTCTC
CTACCTTTGAGCTCCCAATTGGTTTGGCCAATGAGAAGCACCAGTGGGAAAGCACCAGAGAGAGAAGATTGACATAGG
AATATTTCTTCTCCAATTCCTTCTTTGCTGGGTTGGCACTGGACTCATTCCTCCCCGAAAAGTCATACTCCAATCAGA
CTGCCCCTCATACAACTGAAGCTACTTTCTCTGGGGTCAGGTAATCACTCCTCCCCTTGCTCCTTCAGGTCTGCTGCT
GCATTGAGAGTGCTTTTGTATTCCTTGTAGCTTTCTCCTAACATTGCTGACACTTTTGTAAATGTCCCCTTCATGAAA
TTCTTCTATATGCCTCATTTCAGCATGCCATCTGTCTCCTGCCTGGCTGACACAAGGTGATTCAACAGCTCATGAAAG
TCAGCAGGAAGCAAAGATGTGCCTTGCTTCAGCTTGGGGTCTTAATCTTGCTAACTTTTGCAGATAAAGAAAAACAGT
AACTGGGGGAACCACAGTGAAGTCCAGTGCAGAATTCACAGATATCATGGAAAGGTTACTCGGGTGGTCCAGATAGTA
AAATTAACAGTCTAAATTAATCTATCTAAATTTCTGAGGAACGAGAAGCCTTCCCTTGTCATCAGGTGAAGCCAGAAG
AGGGAATATAGCCTCAACCAGAAAAGGGACAGTAATTAAAAGGCTTTTCCCATCCTTGTACAATGGACTGACTTTGCC
TCTTCATAACATCACAATCCTAAAGCAACACAACAATTAATTCTGATATATTAGTAGCTGAAAAAAATTCCCATTTCC
AACTAAGGTAGGTCAGAATTATAGGATAAACCCTGCAGACTTTTTATACTACCCATCCACGCCATTACTCACTGTTAC
CTTTCCAAATACAAAGAGAAGAACTGGTAAAACATAATCATATAAATCTCCATATTCATTTTGAAATATTTGGCATGA
TATTTTCTGTGCTAAAAAGTAATTATTCTTCAAAGAATGATGAGGTCATGTCAGTAAGACACAGGAACCAACTAGAAG
GGGCTTCCCACTGGCCAAATCTGGGGCAAGTTGAGCATCAAAATAAATGATAGTAAAAGATTATAATTCATTGAATAA
GAATCAGCAAATACATACTGATGTAAGTAAATAAGGAAAAGTACAAATCTGTTTCTTGCAGTTGAATGTTAATTAACA
ATTGTAGAAGAAATAACGGAGTTAGAAAAATCACTATTTGGCAATCACCCTAATGACAATTGATTCATACAAGAATCA
TCAATGAGTATTAAAACTCATGGGTGAAAGTTTGATGAGGAATAGGGTATTTATAGCATCTTAAAGTATCTCTTCTCT
ATTAAGTAGAAAATTTAAACAGAAGAAAGTATACTTTGGAGAAATACAGCAGACAATACCTTCAAAGATATCATCAAT
TATGAGACCAACTGATACTATGTGCCTCCTGATAAGATATACTGAAAGGGCCACATTACTTCTTGGTACACAGTCAAA
ATTTTAAAACCAGAATCTAACTACAAGGAAAATCAAATTGAGGACACTCTATAAAATAAGTGGACTGAACTCCTTAAA
AATGTCAATGTCATGAAAGACAAAGAAAGGCTAAAGAATTCCATGAGGTCAAAGAACTATGACAACTAAACACAATTC
TGGATGGAATATCAAATTAAAAAATAACAGATAAATAATATTATTGGGAAAGTTGAATAAATTTGAATATGGACTGTT
TATTAGTTATTAGTATTATAATAGTGTTAATTTTCCTAATTTTGTTAAGACTAGTGTGCCTGTTCCATGAAAATAGAA
AATGTTCTTATTCTCTGAAAATGCATGCTAAAGTATTTAGGGGTGAATGCAACAATGTCTGCAGCTCATTCTTGAATC
AGTTCAAAGAAAAATGAGTTACATTTATATATATATGTATGTATGTAAACAGACATAGATAAAAGTATAGATGTGTGT
GTGTCTTTAGAAAGGGGAGGATTTTTTTTTTTTTTTTGCTGTGTGTTACTGAAGTGCCTATGTCTGCGTGTTCACACT
ATCATATTTTGTATGCCCTGGACTTTATAATTTCTACCTTCAAAATTAGATCTACTGTTGGTAATTAATTCAATATAT
ACTGGTTTTTTAACTACTATTCTCATTTCCTAGCAGTAATCTTCCTGAAAAGTCACAGAAATGATTACATTCCTTGTT
CTTCATAATAATCACTGTTTAATTAAAATAAGAATATTTTAGAAAAGATCTGCGGCATAGTGGTTAAGACCCCAGTAT
TTGATGCTAAACAGATCTGATTTGGATAACAGAAGGTGGCACTTTGCTGTTTAAGCTGGGGACCAGACACTGTGGGTA
TAAATAGTAATTCCAAACACAGCTCCACAGAGCAGCACCCTTATGACAAGGTTTTCATATGTCTATAGTTAAGCCAGA
AAATTAAGAATAATGCCATAAATATTTATAAAGCTGAACATATCCAAGTTAAAGACCTTTATCCTGAAATTGTATCTT
TTAGATTATTTTCTAAAGACTAATACCATTTAATGTTTAAATGTTCTTTGGAAATGATGGTGAGAATACGTGATAATG
GGTCATTGGTTTTAATATTTTATTTAGCCAAGTGGAAAATTGGCAACCTGGTGTCGGTCCTCCCATTTGTATTTTACT
GGTGCATGAAATCCAAAAGTCTAGTAACCATTGGGACAGACAACTCTACTGCATAAGTTTGTATGTTTGTATATCTGT
ATCACAAAGCCCAGACACTCGAACTATATAAACTTGTCGCACTAAAGACAGCAAATATGTCTGGTAATTGCATATTCT
TCATGTGTGCACTGGAATTTCTTATTATATAAGAAAATAAATGTGTTTCTAAACCACCATGAATTGGGTACTGCTGAT
AGTACTATTCTTCCTGGCACATGGAAATATCCCACTGAGGTTGTCAATCAATATTGATTTAATCAGTGTGGCAGCAAA
GGCACTAGAGGAGGAAAATACTCTAGACAATCAAGTATAGATGGGAAAATTGCATCACCAAGAAATTCTTAAAATCAC
AAGTGGATAATTTGCAATATCAATTGATTTTACTCAACTGCTTGAGTCCTTTATCATTCCGTTAGAACACTCCTCATA
TTTGCAGATAACTCAGCCCCTCACCTCAGCAACAAGCAAGAGGACATCATTATAAACTTCCTCAATTTCTTTCCTATC
CAGTGAAAAATATCTAAGTCCCTTTTCCCTTCCCTTTTCCTTCCCTTCCCTTCCTTTTTCTTTCCTTCCATTCTCTTC
CTTTTTCCTTCCTTTTTCTTTCCTTCCATTCTCTTCCTTTTTCCTTCCTTTTTCTTACTTTACCTCCCCCTCCTACCT
TTCCCTTTCTCCCTTCCTCCCTCTCTCCCTTCCTTCCTCTCTCCCTCTCCTTCCTTCCTTCCTTCAATCCATACTTTT
ATATCTTTTCAGAGATCAATTTTTCCCATTCATTTCTACATTCTCAGATACCTTGCTCCATGACTTGTTCTACTTCTC
TTCTATCTTTATTCTTTCCCTCTTGACTGATCCTTTTTGTCTGCCTTTGCATTTTCAAAATCAGTTTTTCTAATCTTA
AAAAATAACTTTGCTCAACCCTGATGCATCTTTATTCACTGTGTCATTATCTCATTATTTTTAAAGGTTAGTTTCTAA
AGCCAGGGCTCTATACTCACTACCTTCACTCTTGGTTAAAATTAAAAGAAATATAGGTCCCAGTGATATTTATGGGAA
AATTTGACTGTGAATAATCTTGTTGATATAAACACTTTGGGTTTTCAAAATGTTATGCTAGCTGTTAACTTTCCCAGT
AACATTATGAGGGAAGGGCTGGAAAATACAAAAGGTAGACATAGGAACCGAGAACACCACCCCTATGGAGCCAAGCTA
TACATACTCTGTCTCTGGTTATCATAAATACATCTCCTATATTCAATGCTTGCTTAGAAAGGCATGATGTCCACGGAG
CAATTCAATTGTTCACTGTGATGGGTTTTTTTGTTTTCTCCTCATGGAATGCAACTTTTATTTAT
SEQ ID NO: 7 >NM_001202435.3 SCN1A [organism = Homo sapiens] [GeneID = 6323]
[transcript = 4]
AAGTGTAGGAGACACACTGCTGGCCTGTGGAAACTCATGGAACTGTTCCTCCAGATTAACACTTCAGGGGTTATGGAA
GCTGGAGGAAGCTGAGCTTTTACTACATCTTTTGGGGGTTTGGGCAATTATGAATAAGGCTGCTGTATACATCCGTGT
GCAGGATTTTGTGTGGACATAAGTTTTCAACTCCTTTGGTTAAATCCTAAGGAATTTCATATGCAGAATAAATGGTAA
TTAAAATGTGCAGGATGACAAGATGGAGCAAACAGTGCTTGTACCACCAGGACCTGACAGCTTCAACTTCTTCACCAG
AGAATCTCTTGCGGCTATTGAAAGACGCATTGCAGAAGAAAAGGCAAAGAATCCCAAACCAGACAAAAAAGATGACGA
CGAAAATGGCCCAAAGCCAAATAGTGACTTGGAAGCTGGAAAGAACCTTCCATTTATTTATGGAGACATTCCTCCAGA
GATGGTGTCAGAGCCCCTGGAGGACCTGGACCCCTACTATATCAATAAGAAAACTTTTATAGTATTGAATAAAGGGAA
GGCCATCTTCCGGTTCAGTGCCACCTCTGCCCTGTACATTTTAACTCCCTTCAATCCTCTTAGGAAAATAGCTATTAA
GATTTTGGTACATTCATTATTCAGCATGCTAATTATGTGCACTATTTTGACAAACTGTGTGTTTATGACAATGAGTAA
CCCTCCTGATTGGACAAAGAATGTAGAATACACCTTCACAGGAATATATACTTTTGAATCACTTATAAAAATTATTGC
AAGGGGATTCTGTTTAGAAGATTTTACTTTCCTTCGGGATCCATGGAACTGGCTCGATTTCACTGTCATTACATTTGC
GTACGTCACAGAGTTTGTGGACCTGGGCAATGTCTCGGCATTGAGAACATTCAGAGTTCTCCGAGCATTGAAGACGAT
TTCAGTCATTCCAGGCCTGAAAACCATTGTGGGAGCCCTGATCCAGTCTGTGAAGAAGCTCTCAGATGTAATGATCCT
GACTGTGTTCTGTCTGAGCGTATTTGCTCTAATTGGGCTGCAGCTGTTCATGGGCAACCTGAGGAATAAATGTATACA
ATGGCCTCCCACCAATGCTTCCTTGGAGGAACATAGTATAGAAAAGAATATAACTGTGAATTATAATGGTACACTTAT
AAATGAAACTGTCTTTGAGTTTGACTGGAAGTCATATATTCAAGATTCAAGATATCATTATTTCCTGGAGGGTTTTTT
AGATGCACTACTATGTGGAAATAGCTCTGATGCAGGCCAATGTCCAGAGGGATATATGTGTGTGAAAGCTGGTAGAAA
TCCCAATTATGGCTACACAAGCTTTGATACCTTCAGTTGGGCTTTTTTGTCCTTGTTTCGACTAATGACTCAGGACTT
CTGGGAAAATCTTTATCAACTGACATTACGTGCTGCTGGGAAAACGTACATGATATTTTTTGTATTGGTCATTTTCTT
GGGCTCATTCTACCTAATAAATTTGATCCTGGCTGTGGTGGCCATGGCCTACGAGGAACAGAATCAGGCCACCTTGGA
AGAAGCAGAACAGAAAGAGGCCGAATTTCAGCAGATGATTGAACAGCTTAAAAAGCAACAGGAGGCAGCTCAGCAGGC
AGCAACGGCAACTGCCTCAGAACATTCCAGAGAGCCCAGTGCAGCAGGCAGGCTCTCAGACAGCTCATCTGAAGCCTC
TAAGTTGAGTTCCAAGAGTGCTAAGGAAAGAAGAAATCGGAGGAAGAAAAGAAAACAGAAAGAGCAGTCTGGTGGGGA
AGAGAAAGATGAGGATGAATTCCAAAAATCTGAATCTGAGGACAGCATCAGGAGGAAAGGTTTTCGCTTCTCCATTGA
AGGGAACCGATTGACATATGAAAAGAGGTACTCCTCCCCACACCAGTCTTTGTTGAGCATCCGTGGCTCCCTATTTTC
ACCAAGGCGAAATAGCAGAACAAGCCTTTTCAGCTTTAGAGGGCGAGCAAAGGATGTGGGATCTGAGAACGACTTCGC
AGATGATGAGCACAGCACCTTTGAGGATAACGAGAGCCGTAGAGATTCCTTGTTTGTGCCCCGACGACACGGAGAGAG
ACGCAACAGCAACCTGAGTCAGACCAGTAGGTCATCCCGGATGCTGGCAGTGTTTCCAGCGAATGGGAAGATGCACAG
CACTGTGGATTGCAATGGTGTGGTTTCCTTGGTTGGTGGACCTTCAGTTCCTACATCGCCTGTTGGACAGCTTCTGCC
AGAGGTGATAATAGATAAGCCAGCTACTGATGACAATGGAACAACCACTGAAACTGAAATGAGAAAGAGAAGGTCAAG
TTCTTTCCACGTTTCCATGGACTTTCTAGAAGATCCTTCCCAAAGGCAACGAGCAATGAGTATAGCCAGCATTCTAAC
AAATACAGTAGAAGAACTTGAAGAATCCAGGCAGAAATGCCCACCCTGTTGGTATAAATTTTCCAACATATTCTTAAT
CTGGGACTGTTCTCCATATTGGTTAAAAGTGAAACATGTTGTCAACCTGGTTGTGATGGACCCATTTGTTGACCTGGC
CATCACCATCTGTATTGTCTTAAATACTCTTTTCATGGCCATGGAGCACTATCCAATGACGGACCATTTCAATAATGT
GCTTACAGTAGGAAACTTGGTTTTCACTGGGATCTTTACAGCAGAAATGTTTCTGAAAATTATTGCCATGGATCCTTA
CTATTATTTCCAAGAAGGCTGGAATATCTTTGACGGTTTTATTGTGACGCTTAGCCTGGTAGAACTTGGACTCGCCAA
TGTGGAAGGATTATCTGTTCTCCGTTCATTTCGATTGCTGCGAGTTTTCAAGTTGGCAAAATCTTGGCCAACGTTAAA
TATGCTAATAAAGATCATCGGCAATTCCGTGGGGGCTCTGGGAAATTTAACCCTCGTCTTGGCCATCATCGTCTTCAT
TTTTGCCGTGGTCGGCATGCAGCTCTTTGGTAAAAGCTACAAAGATTGTGTCTGCAAGATCGCCAGTGATTGTCAACT
CCCACGCTGGCACATGAATGACTTCTTCCACTCCTTCCTGATTGTGTTCCGCGTGCTGTGTGGGGAGTGGATAGAGAC
CATGTGGGACTGTATGGAGGTTGCTGGTCAAGCCATGTGCCTTACTGTCTTCATGATGGTCATGGTGATTGGAAACCT
AGTGGTCCTGAATCTCTTTCTGGCCTTGCTTCTGAGCTCATTTAGTGCAGACAACCTTGCAGCCACTGATGATGATAA
TGAAATGAATAATCTCCAAATTGCTGTGGATAGGATGCACAAAGGAGTAGCTTATGTGAAAAGAAAAATATATGAATT
TATTCAACAGTCCTTCATTAGGAAACAAAAGATTTTAGATGAAATTAAACCACTTGATGATCTAAACAACAAGAAAGA
CAGTTGTATGTCCAATCATACAGCAGAAATTGGGAAAGATCTTGACTATCTTAAAGATGTAAATGGAACTACAAGTGG
TATAGGAACTGGCAGCAGTGTTGAAAAATACATTATTGATGAAAGTGATTACATGTCATTCATAAACAACCCCAGTCT
TACTGTGACTGTACCAATTGCTGTAGGAGAATCTGACTTTGAAAATTTAAACACGGAAGACTTTAGTAGTGAATCGGA
TCTGGAAGAAAGCAAAGAGAAACTGAATGAAAGCAGTAGCTCATCAGAAGGTAGCACTGTGGACATCGGCGCACCTGT
AGAAGAACAGCCCGTAGTGGAACCTGAAGAAACTCTTGAACCAGAAGCTTGTTTCACTGAAGGCTGTGTACAAAGATT
CAAGTGTTGTCAAATCAATGTGGAAGAAGGCAGAGGAAAACAATGGTGGAACCTGAGAAGGACGTGTTTCCGAATAGT
TGAACATAACTGGTTTGAGACCTTCATTGTTTTCATGATTCTCCTTAGTAGTGGTGCTCTGGCATTTGAAGATATATA
TATTGATCAGCGAAAGACGATTAAGACGATGTTGGAATATGCTGACAAGGTTTTCACTTACATTTTCATTCTGGAAAT
GCTTCTAAAATGGGTGGCATATGGCTATCAAACATATTTCACCAATGCCTGGTGTTGGCTGGACTTCTTAATTGTTGA
TGTTTCATTGGTCAGTTTAACAGCAAATGCCTTGGGTTACTCAGAACTTGGAGCCATCAAATCTCTCAGGACACTAAG
AGCTCTGAGACCTCTAAGAGCCTTATCTCGATTTGAAGGGATGAGGGTGGTTGTGAATGCCCTTTTAGGAGCAATTCC
ATCCATCATGAATGTGCTTCTGGTTTGTCTTATATTCTGGCTAATTTTCAGCATCATGGGCGTAAATTTGTTTGCTGG
CAAATTCTACCACTGTATTAACACCACAACTGGTGACAGGTTTGACATCGAAGACGTGAATAATCATACTGATTGCCT
AAAACTAATAGAAAGAAATGAGACTGCTCGATGGAAAAATGTGAAAGTAAACTTTGATAATGTAGGATTTGGGTATCT
CTCTTTGCTTCAAGTTGCCACATTCAAAGGATGGATGGATATAATGTATGCAGCAGTTGATTCCAGAAATGTGGAACT
CCAGCCTAAGTATGAAGAAAGTCTGTACATGTATCTTTACTTTGTTATTTTCATCATCTTTGGGTCCTTCTTCACCTT
GAACCTGTTTATTGGTGTCATCATAGATAATTTCAACCAGCAGAAAAAGAAGTTTGGAGGTCAAGACATCTTTATGAC
AGAAGAACAGAAGAAATACTATAATGCAATGAAAAAATTAGGATCGAAAAAACCGCAAAAGCCTATACCTCGACCAGG
AAACAAATTTCAAGGAATGGTCTTTGACTTCGTAACCAGACAAGTTTTTGACATAAGCATCATGATTCTCATCTGTCT
TAACATGGTCACAATGATGGTGGAAACAGATGACCAGAGTGAATATGTGACTACCATTTTGTCACGCATCAATCTGGT
GTTCATTGTGCTATTTACTGGAGAGTGTGTACTGAAACTCATCTCTCTACGCCATTATTATTTTACCATTGGATGGAA
TATTTTTGATTTTGTGGTTGTCATTCTCTCCATTGTAGGTATGTTTCTTGCCGAGCTGATAGAAAAGTATTTCGTGTC
CCCTACCCTGTTCCGAGTGATCCGTCTTGCTAGGATTGGCCGAATCCTACGTCTGATCAAAGGAGCAAAGGGGATCCG
CACGCTGCTCTTTGCTTTGATGATGTCCCTTCCTGCGTTGTTTAACATCGGCCTCCTACTCTTCCTAGTCATGTTCAT
CTACGCCATCTTTGGGATGTCCAACTTTGCCTATGTTAAGAGGGAAGTTGGGATCGATGACATGTTCAACTTTGAGAC
CTTTGGCAACAGCATGATCTGCCTATTCCAAATTACAACCTCTGCTGGCTGGGATGGATTGCTAGCACCCATTCTCAA
CAGTAAGCCACCCGACTGTGACCCTAATAAAGTTAACCCTGGAAGCTCAGTTAAGGGAGACTGTGGGAACCCATCTGT
TGGAATTTTCTTTTTTGTCAGTTACATCATCATATCCTTCCTGGTTGTGGTGAACATGTACATCGCGGTCATCCTGGA
GAACTTCAGTGTTGCTACTGAAGAAAGTGCAGAGCCTCTGAGTGAGGATGACTTTGAGATGTTCTATGAGGTTTGGGA
GAAGTTTGATCCCGATGCAACTCAGTTCATGGAATTTGAAAAATTATCTCAGTTTGCAGCTGCGCTTGAACCGCCTCT
CAATCTGCCACAACCAAACAAACTCCAGCTCATTGCCATGGATTTGCCCATGGTGAGTGGTGACCGGATCCACTGTCT
TGATATCTTATTTGCTTTTACAAAGCGGGTTCTAGGAGAGAGTGGAGAGATGGATGCTCTACGAATACAGATGGAAGA
GCGATTCATGGCTTCCAATCCTTCCAAGGTCTCCTATCAGCCAATCACTACTACTTTAAAACGAAAACAAGAGGAAGT
ATCTGCTGTCATTATTCAGCGTGCTTACAGACGCCACCTTTTAAAGCGAACTGTAAAACAAGCTTCCTTTACGTACAA
TAAAAACAAAATCAAAGGTGGGGCTAATCTTCTTATAAAAGAAGACATGATAATTGACAGAATAAATGAAAACTCTAT
TACAGAAAAAACTGATCTGACCATGTCCACTGCAGCTTGTCCACCTTCCTATGACCGGGTGACAAAGCCAATTGTGGA
AAAACATGAGCAAGAAGGCAAAGATGAAAAAGCCAAAGGGAAATAAATGAAAATAAATAAAAATAATTGGGTGACAAA
TTGTTTACAGCCTGTGAAGGTGATGTATTTTTATCAACAGGACTCCTTTAGGAGGTCAATGCCAAACTGACTGTTTTT
ACACAAATCTCCTTAAGGTCAGTGCCTACAATAAGACAGTGACCCCTTGTCAGCAAACTGTGACTCTGTGTAAAGGGG
AGATGACCTTGACAGGAGGTTACTGTTCTCACTACCAGCTGACACTGCTGAAGATAAGATGCACAATGGCTAGTCAGA
CTGTAGGGACCAGTTTCAAGGGGTGCAAACCTGTGATTTTGGGGTTGTTTAACATGAAACACTTTAGTGTAGTAATTG
TATCCACTGTTTGCATTTCAACTGCCACATTTGTCACATTTTTATGGAATCTGTTAGTGGATTCATCTTTTTGTTAAT
CCATGTGTTTATTATATGTGACTATTTTTGTAAACGAAGTTTCTGTTGAGAAATAGGCTAAGGACCTCTATAACAGGT
ATGCCACCTGGGGGGTATGGCAACCACATGGCCCTCCCAGCTACACAAAGTCGTGGTTTGCATGAGGGCATGCTGCAC
TTAGAGATCATGCATGAGAAAAAGTCACAAGAAAAACAAATTCTTAAATTTCACCATATTTCTGGGAGGGGTAATTGG
GTGATAAGTGGAGGTGCTTTGTTGATCTTGTTTTGCGAAATCCAGCCCCTAGACCAAGTAGATTATTTGTGGGTAGGC
CAGTAAATCTTAGCAGGTGCAAACTTCATTCAAATGTTTGGAGTCATAAATGTTATGTTTCTTTTTGTTGTATTAAAA
AAAAAACCTGAATAGTGAATATTGCCCCTCACCCTCCACCGCCAGAAGACTGAATTGACCAAAATTACTCTTTATAAA
TTTCTGCTTTTTCCTGCACTTTGTTTAGCCATCTTCGGCTCTCAGCAAGGTTGACACTGTATATGTTAATGAAATGCT
ATTTATTATGTAAATAGTCATTTTACCCTGTGGTGCACGTTTGAGCAAACAAATAATGACCTAAGCACAGTATTTATT
GCATCAAATATGTACCACAAGAAATGTAGAGTGCAAGCTTTACACAGGTAATAAAATGTATTCTGTACCATTTATAGA
TAGTTTGGATGCTATCAATGCATGTTTATATTACCATGCTGCTGTATCTGGTTTCTCTCACTGCTCAGAATCTCATTT
ATGAGAAACCATATGTCAGTGGTAAAGTCAAGGAAATTGTTCAACAGATCTCATTTATTTAAGTCATTAAGCAATAGT
TTGCAGCACTTTAACAGCTTTTTGGTTATTTTTACATTTTAAGTGGATAACATATGGTATATAGCCAGACTGTACAGA
CATGTTTAAAAAAACACACTGCTTAACCTATTAAATATGTGTTTAGAATTTTATAAGCAAATATAAATACTGTAAAAA
GTCACTTTATTTTATTTTTCAGCATTATGTACATAAATATGAAGAGGAAATTATCTTCAGGTTGATATCACAATCACT
TTTCTTACTTTCTGTCCATAGTACTTTTTCATGAAAGAAATTTGCTAAATAAGACATGAAAACAAGACTGGGTAGTTG
TAGATTTCTGCTTTTTAAATTACATTTGCTAATTTTAGATTATTTCACAATTTTAAGGAGCAAAATAGGTTCACGATT
CATATCCAAATTATGCTTTGCAATTGGAAAAGGGTTTAAAATTTTATTTATATTTCTGGTAGTACCTGCACTAACTGA
ATTGAAGGTAGTGCTTATGTTATTTTTGTTCTTTTTTTCTGACTTCGGTTTATGTTTTCATTTCTTTGGAGTAATGCT
GCTCTAGATTGTTCTAAATAGAATGTGGGCTTCATAATTTTTTTTTCCACAAAAACAGAGTAGTCAACTTATATAGTC
AATTACATCAGGACATTTTGTGTTTCTTACAGAAGCAAACCATAGGCTCCTCTTTTCCTTAAAACTACTTAGATAAAC
TGTATTCGTGAACTGCATGCTGGAAAATGCTACTATTATGCTAAATAATGCTAACCAACATTTAAAATGTGCAAAACT
AATAAAGATTACATTTTTTATTTTATTGTTTGCCCAGTCACTTTTTGTTAACAGAATATTCTAATGATATGGAGATTT
TTTACATTACAAATTGGGGGAGAAGGGGAGCGCGCGCGCACACACACACACACACACACACACACACACACACACACA
GAGGCATACCCACGTTGACAACAAAACCTAGGGTAGATATGTCACTGGAGGTAGGGGGTAATGACCTCCCAGAATTAC
AAGCAGCAGGTGTGTTCTCTGTTAGGAGGAAGAACTGGTGTCAGAGGATAGCTAGTGATTCTAGGAGGAAGAGAAGTA
TGGAAGCCAGAGTGATGGTGGATGACCCCTTGAGCTATGAAAAGAAACCCTTAAATCATCATTTAAAAATTTAGAATT
GCCATGTGTGTAGGATACTGTGTTTGCTCCTCCAGAGCCACTCTCTCTGCTTCTGCATCATTCTGTGTGTCCCAGAAG
GGTGACTTCTACACATTGCAAAAATGGGCTCTCCTACCTTTGAGCTCCCAATTGGTTTGGCCAATGAGAAGCACCAGT
GGGAAAGCACCAGAGAGAGAAGATTGACATAGGAATATTTCTTCTCCAATTCCTTCTTTGCTGGGTTGGCACTGGACT
CATTCCTCCCCGAAAAGTCATACTCCAATCAGACTGCCCCTCATACAACTGAAGCTACTTTCTCTGGGGTCAGGTAAT
CACTCCTCCCCTTGCTCCTTCAGGTCTGCTGCTGCATTGAGAGTGCTTTTGTATTCCTTGTAGCTTTCTCCTAACATT
GCTGACACTTTTGTAAATGTCCCCTTCATGAAATTCTTCTATATGCCTCATTTCAGCATGCCATCTGTCTCCTGCCTG
GCTGACACAAGGTGATTCAACAGCTCATGAAAGTCAGCAGGAAGCAAAGATGTGCCTTGCTTCAGCTTGGGGTCTTAA
TCTTGCTAACTTTTGCAGATAAAGAAAAACAGTAACTGGGGGAACCACAGTGAAGTCCAGTGCAGAATTCACAGATAT
CATGGAAAGGTTACTCGGGTGGTCCAGATAGTAAAATTAACAGTCTAAATTAATCTATCTAAATTTCTGAGGAACGAG
AAGCCTTCCCTTGTCATCAGGTGAAGCCAGAAGAGGGAATATAGCCTCAACCAGAAAAGGGACAGTAATTAAAAGGCT
TTTCCCATCCTTGTACAATGGACTGACTTTGCCTCTTCATAACATCACAATCCTAAAGCAACACAACAATTAATTCTG
ATATATTAGTAGCTGAAAAAAATTCCCATTTCCAACTAAGGTAGGTCAGAATTATAGGATAAACCCTGCAGACTTTTT
ATACTACCCATCCACGCCATTACTCACTGTTACCTTTCCAAATACAAAGAGAAGAACTGGTAAAACATAATCATATAA
ATCTCCATATTCATTTTGAAATATTTGGCATGATATTTTCTGTGCTAAAAAGTAATTATTCTTCAAAGAATGATGAGG
TCATGTCAGTAAGACACAGGAACCAACTAGAAGGGGCTTCCCACTGGCCAAATCTGGGGCAAGTTGAGCATCAAAATA
AATGATAGTAAAAGATTATAATTCATTGAATAAGAATCAGCAAATACATACTGATGTAAGTAAATAAGGAAAAGTACA
AATCTGTTTCTTGCAGTTGAATGTTAATTAACAATTGTAGAAGAAATAACGGAGTTAGAAAAATCACTATTTGGCAAT
CACCCTAATGACAATTGATTCATACAAGAATCATCAATGAGTATTAAAACTCATGGGTGAAAGTTTGATGAGGAATAG
GGTATTTATAGCATCTTAAAGTATCTCTTCTCTATTAAGTAGAAAATTTAAACAGAAGAAAGTATACTTTGGAGAAAT
ACAGCAGACAATACCTTCAAAGATATCATCAATTATGAGACCAACTGATACTATGTGCCTCCTGATAAGATATACTGA
AAGGGCCACATTACTTCTTGGTACACAGTCAAAATTTTAAAACCAGAATCTAACTACAAGGAAAATCAAATTGAGGAC
ACTCTATAAAATAAGTGGACTGAACTCCTTAAAAATGTCAATGTCATGAAAGACAAAGAAAGGCTAAAGAATTCCATG
AGGTCAAAGAACTATGACAACTAAACACAATTCTGGATGGAATATCAAATTAAAAAATAACAGATAAATAATATTATT
GGGAAAGTTGAATAAATTTGAATATGGACTGTTTATTAGTTATTAGTATTATAATAGTGTTAATTTTCCTAATTTTGT
TAAGACTAGTGTGCCTGTTCCATGAAAATAGAAAATGTTCTTATTCTCTGAAAATGCATGCTAAAGTATTTAGGGGTG
AATGCAACAATGTCTGCAGCTCATTCTTGAATCAGTTCAAAGAAAAATGAGTTACATTTATATATATATGTATGTATG
TAAACAGACATAGATAAAAGTATAGATGTGTGTGTGTCTTTAGAAAGGGGAGGATTTTTTTTTTTTTTTTGCTGTGTG
TTACTGAAGTGCCTATGTCTGCGTGTTCACACTATCATATTTTGTATGCCCTGGACTTTATAATTTCTACCTTCAAAA
TTAGATCTACTGTTGGTAATTAATTCAATATATACTGGTTTTTTAACTACTATTCTCATTTCCTAGCAGTAATCTTCC
TGAAAAGTCACAGAAATGATTACATTCCTTGTTCTTCATAATAATCACTGTTTAATTAAAATAAGAATATTTTAGAAA
AGATCTGCGGCATAGTGGTTAAGACCCCAGTATTTGATGCTAAACAGATCTGATTTGGATAACAGAAGGTGGCACTTT
GCTGTTTAAGCTGGGGACCAGACACTGTGGGTATAAATAGTAATTCCAAACACAGCTCCACAGAGCAGCACCCTTATG
ACAAGGTTTTCATATGTCTATAGTTAAGCCAGAAAATTAAGAATAATGCCATAAATATTTATAAAGCTGAACATATCC
AAGTTAAAGACCTTTATCCTGAAATTGTATCTTTTAGATTATTTTCTAAAGACTAATACCATTTAATGTTTAAATGTT
CTTTGGAAATGATGGTGAGAATACGTGATAATGGGTCATTGGTTTTAATATTTTATTTAGCCAAGTGGAAAATTGGCA
ACCTGGTGTCGGTCCTCCCATTTGTATTTTACTGGTGCATGAAATCCAAAAGTCTAGTAACCATTGGGACAGACAACT
CTACTGCATAAGTTTGTATGTTTGTATATCTGTATCACAAAGCCCAGACACTCGAACTATATAAACTTGTCGCACTAA
AGACAGCAAATATGTCTGGTAATTGCATATTCTTCATGTGTGCACTGGAATTTCTTATTATATAAGAAAATAAATGTG
TTTCTAAACCACCATGAATTGGGTACTGCTGATAGTACTATTCTTCCTGGCACATGGAAATATCCCACTGAGGTTGTC
AATCAATATTGATTTAATCAGTGTGGCAGCAAAGGCACTAGAGGAGGAAAATACTCTAGACAATCAAGTATAGATGGG
AAAATTGCATCACCAAGAAATTCTTAAAATCACAAGTGGATAATTTGCAATATCAATTGATTTTACTCAACTGCTTGA
GTCCTTTATCATTCCGTTAGAACACTCCTCATATTTGCAGATAACTCAGCCCCTCACCTCAGCAACAAGCAAGAGGAC
ATCATTATAAACTTCCTCAATTTCTTTCCTATCCAGTGAAAAATATCTAAGTCCCTTTTCCCTTCCCTTTTCCTTCCC
TTCCCTTCCTTTTTCTTTCCTTCCATTCTCTTCCTTTTTCCTTCCTTTTTCTTTCCTTCCATTCTCTTCCTTTTTCCT
TCCTTTTTCTTACTTTACCTCCCCCTCCTACCTTTCCCTTTCTCCCTTCCTCCCTCTCTCCCTTCCTTCCTCTCTCCC
TCTCCTTCCTTCCTTCCTTCAATCCATACTTTTATATCTTTTCAGAGATCAATTTTTCCCATTCATTTCTACATTCTC
AGATACCTTGCTCCATGACTTGTTCTACTTCTCTTCTATCTTTATTCTTTCCCTCTTGACTGATCCTTTTTGTCTGCC
TTTGCATTTTCAAAATCAGTTTTTCTAATCTTAAAAAATAACTTTGCTCAACCCTGATGCATCTTTATTCACTGTGTC
ATTATCTCATTATTTTTAAAGGTTAGTTTCTAAAGCCAGGGCTCTATACTCACTACCTTCACTCTTGGTTAAAATTAA
AAGAAATATAGGTCCCAGTGATATTTATGGGAAAATTTGACTGTGAATAATCTTGTTGATATAAACACTTTGGGTTTT
CAAAATGTTATGCTAGCTGTTAACTTTCCCAGTAACATTATGAGGGAAGGGCTGGAAAATACAAAAGGTAGACATAGG
AACCGAGAACACCACCCCTATGGAGCCAAGCTATACATACTCTGTCTCTGGTTATCATAAATACATCTCCTATATTCA
ATGCTTGCTTAGAAAGGCATGATGTCCACGGAGCAATTCAATTGTTCACTGTGATGGGTTTTTTTGTTTTCTCCTCAT
GGAATGCAACTTTTATTTAT
SEQ ID NO: 8 >NM_001353955.2 SCN1A [organism = Homo sapiens] [GeneID = 6323]
[transcript = 11]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGAATTTCATATGCAGAATAAATGGTAATTAAAATGTGCAGGATGACAAGATGGAGCAAACAG
TGCTTGTACCACCAGGACCTGACAGCTTCAACTTCTTCACCAGAGAATCTCTTGCGGCTATTGAAAGACGCATTGCAG
AAGAAAAGGCAAAGAATCCCAAACCAGACAAAAAAGATGACGACGAAAATGGCCCAAAGCCAAATAGTGACTTGGAAG
CTGGAAAGAACCTTCCATTTATTTATGGAGACATTCCTCCAGAGATGGTGTCAGAGCCCCTGGAGGACCTGGACCCCT
ACTATATCAATAAGAAAACTTTTATAGTATTGAATAAAGGGAAGGCCATCTTCCGGTTCAGTGCCACCTCTGCCCTGT
ACATTTTAACTCCCTTCAATCCTCTTAGGAAAATAGCTATTAAGATTTTGGTACATTCATTATTCAGCATGCTAATTA
TGTGCACTATTTTGACAAACTGTGTGTTTATGACAATGAGTAACCCTCCTGATTGGACAAAGAATGTAGAATACACCT
TCACAGGAATATATACTTTTGAATCACTTATAAAAATTATTGCAAGGGGATTCTGTTTAGAAGATTTTACTTTCCTTC
GGGATCCATGGAACTGGCTCGATTTCACTGTCATTACATTTGCGTACGTCACAGAGTTTGTGGACCTGGGCAATGTCT
CGGCATTGAGAACATTCAGAGTTCTCCGAGCATTGAAGACGATTTCAGTCATTCCAGGCCTGAAAACCATTGTGGGAG
CCCTGATCCAGTCTGTGAAGAAGCTCTCAGATGTAATGATCCTGACTGTGTTCTGTCTGAGCGTATTTGCTCTAATTG
GGCTGCAGCTGTTCATGGGCAACCTGAGGAATAAATGTATACAATGGCCTCCCACCAATGCTTCCTTGGAGGAACATA
GTATAGAAAAGAATATAACTGTGAATTATAATGGTACACTTATAAATGAAACTGTCTTTGAGTTTGACTGGAAGTCAT
ATATTCAAGATTCAAGATATCATTATTTCCTGGAGGGTTTTTTAGATGCACTACTATGTGGAAATAGCTCTGATGCAG
GCCAATGTCCAGAGGGATATATGTGTGTGAAAGCTGGTAGAAATCCCAATTATGGCTACACAAGCTTTGATACCTTCA
GTTGGGCTTTTTTGTCCTTGTTTCGACTAATGACTCAGGACTTCTGGGAAAATCTTTATCAACTGACATTACGTGCTG
CTGGGAAAACGTACATGATATTTTTTGTATTGGTCATTTTCTTGGGCTCATTCTACCTAATAAATTTGATCCTGGCTG
TGGTGGCCATGGCCTACGAGGAACAGAATCAGGCCACCTTGGAAGAAGCAGAACAGAAAGAGGCCGAATTTCAGCAGA
TGATTGAACAGCTTAAAAAGCAACAGGAGGCAGCTCAGGCAGCAACGGCAACTGCCTCAGAACATTCCAGAGAGCCCA
GTGCAGCAGGCAGGCTCTCAGACAGCTCATCTGAAGCCTCTAAGTTGAGTTCCAAGAGTGCTAAGGAAAGAAGAAATC
GGAGGAAGAAAAGAAAACAGAAAGAGCAGTCTGGTGGGGAAGAGAAAGATGAGGATGAATTCCAAAAATCTGAATCTG
AGGACAGCATCAGGAGGAAAGGTTTTCGCTTCTCCATTGAAGGGAACCGATTGACATATGAAAAGAGGTACTCCTCCC
CACACCAGTCTTTGTTGAGCATCCGTGGCTCCCTATTTTCACCAAGGCGAAATAGCAGAACAAGCCTTTTCAGCTTTA
GAGGGCGAGCAAAGGATGTGGGATCTGAGAACGACTTCGCAGATGATGAGCACAGCACCTTTGAGGATAACGAGAGCC
GTAGAGATTCCTTGTTTGTGCCCCGACGACACGGAGAGAGACGCAACAGCAACCTGAGTCAGACCAGTAGGTCATCCC
GGATGCTGGCAGTGTTTCCAGCGAATGGGAAGATGCACAGCACTGTGGATTGCAATGGTGTGGTTTCCTTGGTTGGTG
GACCTTCAGTTCCTACATCGCCTGTTGGACAGCTTCTGCCAGAGGGAACAACCACTGAAACTGAAATGAGAAAGAGAA
GGTCAAGTTCTTTCCACGTTTCCATGGACTTTCTAGAAGATCCTTCCCAAAGGCAACGAGCAATGAGTATAGCCAGCA
TTCTAACAAATACAGTAGAAGAACTTGAAGAATCCAGGCAGAAATGCCCACCCTGTTGGTATAAATTTTCCAACATAT
TCTTAATCTGGGACTGTTCTCCATATTGGTTAAAAGTGAAACATGTTGTCAACCTGGTTGTGATGGACCCATTTGTTG
ACCTGGCCATCACCATCTGTATTGTCTTAAATACTCTTTTCATGGCCATGGAGCACTATCCAATGACGGACCATTTCA
ATAATGTGCTTACAGTAGGAAACTTGGTTTTCACTGGGATCTTTACAGCAGAAATGTTTCTGAAAATTATTGCCATGG
ATCCTTACTATTATTTCCAAGAAGGCTGGAATATCTTTGACGGTTTTATTGTGACGCTTAGCCTGGTAGAACTTGGAC
TCGCCAATGTGGAAGGATTATCTGTTCTCCGTTCATTTCGATTGCTGCGAGTTTTCAAGTTGGCAAAATCTTGGCCAA
CGTTAAATATGCTAATAAAGATCATCGGCAATTCCGTGGGGGCTCTGGGAAATTTAACCCTCGTCTTGGCCATCATCG
TCTTCATTTTTGCCGTGGTCGGCATGCAGCTCTTTGGTAAAAGCTACAAAGATTGTGTCTGCAAGATCGCCAGTGATT
GTCAACTCCCACGCTGGCACATGAATGACTTCTTCCACTCCTTCCTGATTGTGTTCCGCGTGCTGTGTGGGGAGTGGA
TAGAGACCATGTGGGACTGTATGGAGGTTGCTGGTCAAGCCATGTGCCTTACTGTCTTCATGATGGTCATGGTGATTG
GAAACCTAGTGGTCCTGAATCTCTTTCTGGCCTTGCTTCTGAGCTCATTTAGTGCAGACAACCTTGCAGCCACTGATG
ATGATAATGAAATGAATAATCTCCAAATTGCTGTGGATAGGATGCACAAAGGAGTAGCTTATGTGAAAAGAAAAATAT
ATGAATTTATTCAACAGTCCTTCATTAGGAAACAAAAGATTTTAGATGAAATTAAACCACTTGATGATCTAAACAACA
AGAAAGACAGTTGTATGTCCAATCATACAGCAGAAATTGGGAAAGATCTTGACTATCTTAAAGATGTAAATGGAACTA
CAAGTGGTATAGGAACTGGCAGCAGTGTTGAAAAATACATTATTGATGAAAGTGATTACATGTCATTCATAAACAACC
CCAGTCTTACTGTGACTGTACCAATTGCTGTAGGAGAATCTGACTTTGAAAATTTAAACACGGAAGACTTTAGTAGTG
AATCGGATCTGGAAGAAAGCAAAGAGAAACTGAATGAAAGCAGTAGCTCATCAGAAGGTAGCACTGTGGACATCGGCG
CACCTGTAGAAGAACAGCCCGTAGTGGAACCTGAAGAAACTCTTGAACCAGAAGCTTGTTTCACTGAAGGCTGTGTAC
AAAGATTCAAGTGTTGTCAAATCAATGTGGAAGAAGGCAGAGGAAAACAATGGTGGAACCTGAGAAGGACGTGTTTCC
GAATAGTTGAACATAACTGGTTTGAGACCTTCATTGTTTTCATGATTCTCCTTAGTAGTGGTGCTCTGGCATTTGAAG
ATATATATATTGATCAGCGAAAGACGATTAAGACGATGTTGGAATATGCTGACAAGGTTTTCACTTACATTTTCATTC
TGGAAATGCTTCTAAAATGGGTGGCATATGGCTATCAAACATATTTCACCAATGCCTGGTGTTGGCTGGACTTCTTAA
TTGTTGATGTTTCATTGGTCAGTTTAACAGCAAATGCCTTGGGTTACTCAGAACTTGGAGCCATCAAATCTCTCAGGA
CACTAAGAGCTCTGAGACCTCTAAGAGCCTTATCTCGATTTGAAGGGATGAGGGTGGTTGTGAATGCCCTTTTAGGAG
CAATTCCATCCATCATGAATGTGCTTCTGGTTTGTCTTATATTCTGGCTAATTTTCAGCATCATGGGCGTAAATTTGT
TTGCTGGCAAATTCTACCACTGTATTAACACCACAACTGGTGACAGGTTTGACATCGAAGACGTGAATAATCATACTG
ATTGCCTAAAACTAATAGAAAGAAATGAGACTGCTCGATGGAAAAATGTGAAAGTAAACTTTGATAATGTAGGATTTG
GGTATCTCTCTTTGCTTCAAGTTGCCACATTCAAAGGATGGATGGATATAATGTATGCAGCAGTTGATTCCAGAAATG
TGGAACTCCAGCCTAAGTATGAAGAAAGTCTGTACATGTATCTTTACTTTGTTATTTTCATCATCTTTGGGTCCTTCT
TCACCTTGAACCTGTTTATTGGTGTCATCATAGATAATTTCAACCAGCAGAAAAAGAAGTTTGGAGGTCAAGACATCT
TTATGACAGAAGAACAGAAGAAATACTATAATGCAATGAAAAAATTAGGATCGAAAAAACCGCAAAAGCCTATACCTC
GACCAGGAAACAAATTTCAAGGAATGGTCTTTGACTTCGTAACCAGACAAGTTTTTGACATAAGCATCATGATTCTCA
TCTGTCTTAACATGGTCACAATGATGGTGGAAACAGATGACCAGAGTGAATATGTGACTACCATTTTGTCACGCATCA
ATCTGGTGTTCATTGTGCTATTTACTGGAGAGTGTGTACTGAAACTCATCTCTCTACGCCATTATTATTTTACCATTG
GATGGAATATTTTTGATTTTGTGGTTGTCATTCTCTCCATTGTAGGTATGTTTCTTGCCGAGCTGATAGAAAAGTATT
TCGTGTCCCCTACCCTGTTCCGAGTGATCCGTCTTGCTAGGATTGGCCGAATCCTACGTCTGATCAAAGGAGCAAAGG
GGATCCGCACGCTGCTCTTTGCTTTGATGATGTCCCTTCCTGCGTTGTTTAACATCGGCCTCCTACTCTTCCTAGTCA
TGTTCATCTACGCCATCTTTGGGATGTCCAACTTTGCCTATGTTAAGAGGGAAGTTGGGATCGATGACATGTTCAACT
TTGAGACCTTTGGCAACAGCATGATCTGCCTATTCCAAATTACAACCTCTGCTGGCTGGGATGGATTGCTAGCACCCA
TTCTCAACAGTAAGCCACCCGACTGTGACCCTAATAAAGTTAACCCTGGAAGCTCAGTTAAGGGAGACTGTGGGAACC
CATCTGTTGGAATTTTCTTTTTTGTCAGTTACATCATCATATCCTTCCTGGTTGTGGTGAACATGTACATCGCGGTCA
TCCTGGAGAACTTCAGTGTTGCTACTGAAGAAAGTGCAGAGCCTCTGAGTGAGGATGACTTTGAGATGTTCTATGAGG
TTTGGGAGAAGTTTGATCCCGATGCAACTCAGTTCATGGAATTTGAAAAATTATCTCAGTTTGCAGCTGCGCTTGAAC
CGCCTCTCAATCTGCCACAACCAAACAAACTCCAGCTCATTGCCATGGATTTGCCCATGGTGAGTGGTGACCGGATCC
ACTGTCTTGATATCTTATTTGCTTTTACAAAGCGGGTTCTAGGAGAGAGTGGAGAGATGGATGCTCTACGAATACAGA
TGGAAGAGCGATTCATGGCTTCCAATCCTTCCAAGGTCTCCTATCAGCCAATCACTACTACTTTAAAACGAAAACAAG
AGGAAGTATCTGCTGTCATTATTCAGCGTGCTTACAGACGCCACCTTTTAAAGCGAACTGTAAAACAAGCTTCCTTTA
CGTACAATAAAAACAAAATCAAAGGTGGGGCTAATCTTCTTATAAAAGAAGACATGATAATTGACAGAATAAATGAAA
ACTCTATTACAGAAAAAACTGATCTGACCATGTCCACTGCAGCTTGTCCACCTTCCTATGACCGGGTGACAAAGCCAA
TTGTGGAAAAACATGAGCAAGAAGGCAAAGATGAAAAAGCCAAAGGGAAATAAATGAAAATAAATAAAAATAATTGGG
TGACAAATTGTTTACAGCCTGTGAAGGTGATGTATTTTTATCAACAGGACTCCTTTAGGAGGTCAATGCCAAACTGAC
TGTTTTTACACAAATCTCCTTAAGGTCAGTGCCTACAATAAGACAGTGACCCCTTGTCAGCAAACTGTGACTCTGTGT
AAAGGGGAGATGACCTTGACAGGAGGTTACTGTTCTCACTACCAGCTGACACTGCTGAAGATAAGATGCACAATGGCT
AGTCAGACTGTAGGGACCAGTTTCAAGGGGTGCAAACCTGTGATTTTGGGGTTGTTTAACATGAAACACTTTAGTGTA
GTAATTGTATCCACTGTTTGCATTTCAACTGCCACATTTGTCACATTTTTATGGAATCTGTTAGTGGATTCATCTTTT
TGTTAATCCATGTGTTTATTATATGTGACTATTTTTGTAAACGAAGTTTCTGTTGAGAAATAGGCTAAGGACCTCTAT
AACAGGTATGCCACCTGGGGGGTATGGCAACCACATGGCCCTCCCAGCTACACAAAGTCGTGGTTTGCATGAGGGCAT
GCTGCACTTAGAGATCATGCATGAGAAAAAGTCACAAGAAAAACAAATTCTTAAATTTCACCATATTTCTGGGAGGGG
TAATTGGGTGATAAGTGGAGGTGCTTTGTTGATCTTGTTTTGCGAAATCCAGCCCCTAGACCAAGTAGATTATTTGTG
GGTAGGCCAGTAAATCTTAGCAGGTGCAAACTTCATTCAAATGTTTGGAGTCATAAATGTTATGTTTCTTTTTGTTGT
ATTAAAAAAAAAACCTGAATAGTGAATATTGCCCCTCACCCTCCACCGCCAGAAGACTGAATTGACCAAAATTACTCT
TTATAAATTTCTGCTTTTTCCTGCACTTTGTTTAGCCATCTTCGGCTCTCAGCAAGGTTGACACTGTATATGTTAATG
AAATGCTATTTATTATGTAAATAGTCATTTTACCCTGTGGTGCACGTTTGAGCAAACAAATAATGACCTAAGCACAGT
ATTTATTGCATCAAATATGTACCACAAGAAATGTAGAGTGCAAGCTTTACACAGGTAATAAAATGTATTCTGTACCAT
TTATAGATAGTTTGGATGCTATCAATGCATGTTTATATTACCATGCTGCTGTATCTGGTTTCTCTCACTGCTCAGAAT
CTCATTTATGAGAAACCATATGTCAGTGGTAAAGTCAAGGAAATTGTTCAACAGATCTCATTTATTTAAGTCATTAAG
CAATAGTTTGCAGCACTTTAACAGCTTTTTGGTTATTTTTACATTTTAAGTGGATAACATATGGTATATAGCCAGACT
GTACAGACATGTTTAAAAAAACACACTGCTTAACCTATTAAATATGTGTTTAGAATTTTATAAGCAAATATAAATACT
GTAAAAAGTCACTTTATTTTATTTTTCAGCATTATGTACATAAATATGAAGAGGAAATTATCTTCAGGTTGATATCAC
AATCACTTTTCTTACTTTCTGTCCATAGTACTTTTTCATGAAAGAAATTTGCTAAATAAGACATGAAAACAAGACTGG
GTAGTTGTAGATTTCTGCTTTTTAAATTACATTTGCTAATTTTAGATTATTTCACAATTTTAAGGAGCAAAATAGGTT
CACGATTCATATCCAAATTATGCTTTGCAATTGGAAAAGGGTTTAAAATTTTATTTATATTTCTGGTAGTACCTGCAC
TAACTGAATTGAAGGTAGTGCTTATGTTATTTTTGTTCTTTTTTTCTGACTTCGGTTTATGTTTTCATTTCTTTGGAG
TAATGCTGCTCTAGATTGTTCTAAATAGAATGTGGGCTTCATAATTTTTTTTTCCACAAAAACAGAGTAGTCAACTTA
TATAGTCAATTACATCAGGACATTTTGTGTTTCTTACAGAAGCAAACCATAGGCTCCTCTTTTCCTTAAAACTACTTA
GATAAACTGTATTCGTGAACTGCATGCTGGAAAATGCTACTATTATGCTAAATAATGCTAACCAACATTTAAAATGTG
CAAAACTAATAAAGATTACATTTTTTATTTTATTGTTTGCCCAGTCACTTTTTGTTAACAGAATATTCTAATGATATG
GAGATTTTTTACATTACAAATTGGGGGAGAAGGGGAGCGCGCGCGCACACACACACACACACACACACACACACACAC
ACACACAGAGGCATACCCACGTTGACAACAAAACCTAGGGTAGATATGTCACTGGAGGTAGGGGGTAATGACCTCCCA
GAATTACAAGCAGCAGGTGTGTTCTCTGTTAGGAGGAAGAACTGGTGTCAGAGGATAGCTAGTGATTCTAGGAGGAAG
AGAAGTATGGAAGCCAGAGTGATGGTGGATGACCCCTTGAGCTATGAAAAGAAACCCTTAAATCATCATTTAAAAATT
TAGAATTGCCATGTGTGTAGGATACTGTGTTTGCTCCTCCAGAGCCACTCTCTCTGCTTCTGCATCATTCTGTGTGTC
CCAGAAGGGTGACTTCTACACATTGCAAAAATGGGCTCTCCTACCTTTGAGCTCCCAATTGGTTTGGCCAATGAGAAG
CACCAGTGGGAAAGCACCAGAGAGAGAAGATTGACATAGGAATATTTCTTCTCCAATTCCTTCTTTGCTGGGTTGGCA
CTGGACTCATTCCTCCCCGAAAAGTCATACTCCAATCAGACTGCCCCTCATACAACTGAAGCTACTTTCTCTGGGGTC
AGGTAATCACTCCTCCCCTTGCTCCTTCAGGTCTGCTGCTGCATTGAGAGTGCTTTTGTATTCCTTGTAGCTTTCTCC
TAACATTGCTGACACTTTTGTAAATGTCCCCTTCATGAAATTCTTCTATATGCCTCATTTCAGCATGCCATCTGTCTC
CTGCCTGGCTGACACAAGGTGATTCAACAGCTCATGAAAGTCAGCAGGAAGCAAAGATGTGCCTTGCTTCAGCTTGGG
GTCTTAATCTTGCTAACTTTTGCAGATAAAGAAAAACAGTAACTGGGGGAACCACAGTGAAGTCCAGTGCAGAATTCA
CAGATATCATGGAAAGGTTACTCGGGTGGTCCAGATAGTAAAATTAACAGTCTAAATTAATCTATCTAAATTTCTGAG
GAACGAGAAGCCTTCCCTTGTCATCAGGTGAAGCCAGAAGAGGGAATATAGCCTCAACCAGAAAAGGGACAGTAATTA
AAAGGCTTTTCCCATCCTTGTACAATGGACTGACTTTGCCTCTTCATAACATCACAATCCTAAAGCAACACAACAATT
AATTCTGATATATTAGTAGCTGAAAAAAATTCCCATTTCCAACTAAGGTAGGTCAGAATTATAGGATAAACCCTGCAG
ACTTTTTATACTACCCATCCACGCCATTACTCACTGTTACCTTTCCAAATACAAAGAGAAGAACTGGTAAAACATAAT
CATATAAATCTCCATATTCATTTTGAAATATTTGGCATGATATTTTCTGTGCTAAAAAGTAATTATTCTTCAAAGAAT
GATGAGGTCATGTCAGTAAGACACAGGAACCAACTAGAAGGGGCTTCCCACTGGCCAAATCTGGGGCAAGTTGAGCAT
CAAAATAAATGATAGTAAAAGATTATAATTCATTGAATAAGAATCAGCAAATACATACTGATGTAAGTAAATAAGGAA
AAGTACAAATCTGTTTCTTGCAGTTGAATGTTAATTAACAATTGTAGAAGAAATAACGGAGTTAGAAAAATCACTATT
TGGCAATCACCCTAATGACAATTGATTCATACAAGAATCATCAATGAGTATTAAAACTCATGGGTGAAAGTTTGATGA
GGAATAGGGTATTTATAGCATCTTAAAGTATCTCTTCTCTATTAAGTAGAAAATTTAAACAGAAGAAAGTATACTTTG
GAGAAATACAGCAGACAATACCTTCAAAGATATCATCAATTATGAGACCAACTGATACTATGTGCCTCCTGATAAGAT
ATACTGAAAGGGCCACATTACTTCTTGGTACACAGTCAAAATTTTAAAACCAGAATCTAACTACAAGGAAAATCAAAT
TGAGGACACTCTATAAAATAAGTGGACTGAACTCCTTAAAAATGTCAATGTCATGAAAGACAAAGAAAGGCTAAAGAA
TTCCATGAGGTCAAAGAACTATGACAACTAAACACAATTCTGGATGGAATATCAAATTAAAAAATAACAGATAAATAA
TATTATTGGGAAAGTTGAATAAATTTGAATATGGACTGTTTATTAGTTATTAGTATTATAATAGTGTTAATTTTCCTA
ATTTTGTTAAGACTAGTGTGCCTGTTCCATGAAAATAGAAAATGTTCTTATTCTCTGAAAATGCATGCTAAAGTATTT
AGGGGTGAATGCAACAATGTCTGCAGCTCATTCTTGAATCAGTTCAAAGAAAAATGAGTTACATTTATATATATATGT
ATGTATGTAAACAGACATAGATAAAAGTATAGATGTGTGTGTGTCTTTAGAAAGGGGAGGATTTTTTTTTTTTTTTTG
CTGTGTGTTACTGAAGTGCCTATGTCTGCGTGTTCACACTATCATATTTTGTATGCCCTGGACTTTATAATTTCTACC
TTCAAAATTAGATCTACTGTTGGTAATTAATTCAATATATACTGGTTTTTTAACTACTATTCTCATTTCCTAGCAGTA
ATCTTCCTGAAAAGTCACAGAAATGATTACATTCCTTGTTCTTCATAATAATCACTGTTTAATTAAAATAAGAATATT
TTAGAAAAGATCTGCGGCATAGTGGTTAAGACCCCAGTATTTGATGCTAAACAGATCTGATTTGGATAACAGAAGGTG
GCACTTTGCTGTTTAAGCTGGGGACCAGACACTGTGGGTATAAATAGTAATTCCAAACACAGCTCCACAGAGCAGCAC
CCTTATGACAAGGTTTTCATATGTCTATAGTTAAGCCAGAAAATTAAGAATAATGCCATAAATATTTATAAAGCTGAA
CATATCCAAGTTAAAGACCTTTATCCTGAAATTGTATCTTTTAGATTATTTTCTAAAGACTAATACCATTTAATGTTT
AAATGTTCTTTGGAAATGATGGTGAGAATACGTGATAATGGGTCATTGGTTTTAATATTTTATTTAGCCAAGTGGAAA
ATTGGCAACCTGGTGTCGGTCCTCCCATTTGTATTTTACTGGTGCATGAAATCCAAAAGTCTAGTAACCATTGGGACA
GACAACTCTACTGCATAAGTTTGTATGTTTGTATATCTGTATCACAAAGCCCAGACACTCGAACTATATAAACTTGTC
GCACTAAAGACAGCAAATATGTCTGGTAATTGCATATTCTTCATGTGTGCACTGGAATTTCTTATTATATAAGAAAAT
AAATGTGTTTCTAAACCACCATGAATTGGGTACTGCTGATAGTACTATTCTTCCTGGCACATGGAAATATCCCACTGA
GGTTGTCAATCAATATTGATTTAATCAGTGTGGCAGCAAAGGCACTAGAGGAGGAAAATACTCTAGACAATCAAGTAT
AGATGGGAAAATTGCATCACCAAGAAATTCTTAAAATCACAAGTGGATAATTTGCAATATCAATTGATTTTACTCAAC
TGCTTGAGTCCTTTATCATTCCGTTAGAACACTCCTCATATTTGCAGATAACTCAGCCCCTCACCTCAGCAACAAGCA
AGAGGACATCATTATAAACTTCCTCAATTTCTTTCCTATCCAGTGAAAAATATCTAAGTCCCTTTTCCCTTCCCTTTT
CCTTCCCTTCCCTTCCTTTTTCTTTCCTTCCATTCTCTTCCTTTTTCCTTCCTTTTTCTTTCCTTCCATTCTCTTCCT
TTTTCCTTCCTTTTTCTTACTTTACCTCCCCCTCCTACCTTTCCCTTTCTCCCTTCCTCCCTCTCTCCCTTCCTTCCT
CTCTCCCTCTCCTTCCTTCCTTCCTTCAATCCATACTTTTATATCTTTTCAGAGATCAATTTTTCCCATTCATTTCTA
CATTCTCAGATACCTTGCTCCATGACTTGTTCTACTTCTCTTCTATCTTTATTCTTTCCCTCTTGACTGATCCTTTTT
GTCTGCCTTTGCATTTTCAAAATCAGTTTTTCTAATCTTAAAAAATAACTTTGCTCAACCCTGATGCATCTTTATTCA
CTGTGTCATTATCTCATTATTTTTAAAGGTTAGTTTCTAAAGCCAGGGCTCTATACTCACTACCTTCACTCTTGGTTA
AAATTAAAAGAAATATAGGTCCCAGTGATATTTATGGGAAAATTTGACTGTGAATAATCTTGTTGATATAAACACTTT
GGGTTTTCAAAATGTTATGCTAGCTGTTAACTTTCCCAGTAACATTATGAGGGAAGGGCTGGAAAATACAAAAGGTAG
ACATAGGAACCGAGAACACCACCCCTATGGAGCCAAGCTATACATACTCTGTCTCTGGTTATCATAAATACATCTCCT
ATATTCAATGCTTGCTTAGAAAGGCATGATGTCCACGGAGCAATTCAATTGTTCACTGTGATGGGTTTTTTTGTTTTC
TCCTCATGGAATGCAACTTTTATTTAT
SEQ ID NO: 9 >NM_001353957.2 SCN1A [organism = Homo sapiens] [GeneID = 6323]
[transcript = 12]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGAATTTCATATGCAGAATAAATGGTAATTAAAATGTGCAGGATGACAAGATGGAGCAAACAG
TGCTTGTACCACCAGGACCTGACAGCTTCAACTTCTTCACCAGAGAATCTCTTGCGGCTATTGAAAGACGCATTGCAG
AAGAAAAGGCAAAGAATCCCAAACCAGACAAAAAAGATGACGACGAAAATGGCCCAAAGCCAAATAGTGACTTGGAAG
CTGGAAAGAACCTTCCATTTATTTATGGAGACATTCCTCCAGAGATGGTGTCAGAGCCCCTGGAGGACCTGGACCCCT
ACTATATCAATAAGAAAACTTTTATAGTATTGAATAAAGGGAAGGCCATCTTCCGGTTCAGTGCCACCTCTGCCCTGT
ACATTTTAACTCCCTTCAATCCTCTTAGGAAAATAGCTATTAAGATTTTGGTACATTCATTATTCAGCATGCTAATTA
TGTGCACTATTTTGACAAACTGTGTGTTTATGACAATGAGTAACCCTCCTGATTGGACAAAGAATGTAGAATACACCT
TCACAGGAATATATACTTTTGAATCACTTATAAAAATTATTGCAAGGGGATTCTGTTTAGAAGATTTTACTTTCCTTC
GGGATCCATGGAACTGGCTCGATTTCACTGTCATTACATTTGCGTACGTCACAGAGTTTGTGGACCTGGGCAATGTCT
CGGCATTGAGAACATTCAGAGTTCTCCGAGCATTGAAGACGATTTCAGTCATTCCAGGCCTGAAAACCATTGTGGGAG
CCCTGATCCAGTCTGTGAAGAAGCTCTCAGATGTAATGATCCTGACTGTGTTCTGTCTGAGCGTATTTGCTCTAATTG
GGCTGCAGCTGTTCATGGGCAACCTGAGGAATAAATGTATACAATGGCCTCCCACCAATGCTTCCTTGGAGGAACATA
GTATAGAAAAGAATATAACTGTGAATTATAATGGTACACTTATAAATGAAACTGTCTTTGAGTTTGACTGGAAGTCAT
ATATTCAAGATTCAAGATATCATTATTTCCTGGAGGGTTTTTTAGATGCACTACTATGTGGAAATAGCTCTGATGCAG
GCCAATGTCCAGAGGGATATATGTGTGTGAAAGCTGGTAGAAATCCCAATTATGGCTACACAAGCTTTGATACCTTCA
GTTGGGCTTTTTTGTCCTTGTTTCGACTAATGACTCAGGACTTCTGGGAAAATCTTTATCAACTGACATTACGTGCTG
CTGGGAAAACGTACATGATATTTTTTGTATTGGTCATTTTCTTGGGCTCATTCTACCTAATAAATTTGATCCTGGCTG
TGGTGGCCATGGCCTACGAGGAACAGAATCAGGCCACCTTGGAAGAAGCAGAACAGAAAGAGGCCGAATTTCAGCAGA
TGATTGAACAGCTTAAAAAGCAACAGGAGGCAGCTCAGCAGGCAGCAACGGCAACTGCCTCAGAACATTCCAGAGAGC
CCAGTGCAGCAGGCAGGCTCTCAGACAGCTCATCTGAAGCCTCTAAGTTGAGTTCCAAGAGTGCTAAGGAAAGAAGAA
ATCGGAGGAAGAAAAGAAAACAGAAAGAGCAGTCTGGTGGGGAAGAGAAAGATGAGGATGAATTCCAAAAATCTGAAT
CTGAGGACAGCATCAGGAGGAAAGGTTTTCGCTTCTCCATTGAAGGGAACCGATTGACATATGAAAAGAGGTACTCCT
CCCCACACCAGTCTTTGTTGAGCATCCGTGGCTCCCTATTTTCACCAAGGCGAAATAGCAGAACAAGCCTTTTCAGCT
TTAGAGGGCGAGCAAAGGATGTGGGATCTGAGAACGACTTCGCAGATGATGAGCACAGCACCTTTGAGGATAACGAGA
GCCGTAGAGATTCCTTGTTTGTGCCCCGACGACACGGAGAGAGACGCAACAGCAACCTGAGTCAGACCAGTAGGTCAT
CCCGGATGCTGGCAGTGTTTCCAGCGAATGGGAAGATGCACAGCACTGTGGATTGCAATGGTGTGGTTTCCTTGGGAA
CAACCACTGAAACTGAAATGAGAAAGAGAAGGTCAAGTTCTTTCCACGTTTCCATGGACTTTCTAGAAGATCCTTCCC
AAAGGCAACGAGCAATGAGTATAGCCAGCATTCTAACAAATACAGTAGAAGAACTTGAAGAATCCAGGCAGAAATGCC
CACCCTGTTGGTATAAATTTTCCAACATATTCTTAATCTGGGACTGTTCTCCATATTGGTTAAAAGTGAAACATGTTG
TCAACCTGGTTGTGATGGACCCATTTGTTGACCTGGCCATCACCATCTGTATTGTCTTAAATACTCTTTTCATGGCCA
TGGAGCACTATCCAATGACGGACCATTTCAATAATGTGCTTACAGTAGGAAACTTGGTTTTCACTGGGATCTTTACAG
CAGAAATGTTTCTGAAAATTATTGCCATGGATCCTTACTATTATTTCCAAGAAGGCTGGAATATCTTTGACGGTTTTA
TTGTGACGCTTAGCCTGGTAGAACTTGGACTCGCCAATGTGGAAGGATTATCTGTTCTCCGTTCATTTCGATTGCTGC
GAGTTTTCAAGTTGGCAAAATCTTGGCCAACGTTAAATATGCTAATAAAGATCATCGGCAATTCCGTGGGGGCTCTGG
GAAATTTAACCCTCGTCTTGGCCATCATCGTCTTCATTTTTGCCGTGGTCGGCATGCAGCTCTTTGGTAAAAGCTACA
AAGATTGTGTCTGCAAGATCGCCAGTGATTGTCAACTCCCACGCTGGCACATGAATGACTTCTTCCACTCCTTCCTGA
TTGTGTTCCGCGTGCTGTGTGGGGAGTGGATAGAGACCATGTGGGACTGTATGGAGGTTGCTGGTCAAGCCATGTGCC
TTACTGTCTTCATGATGGTCATGGTGATTGGAAACCTAGTGGTCCTGAATCTCTTTCTGGCCTTGCTTCTGAGCTCAT
TTAGTGCAGACAACCTTGCAGCCACTGATGATGATAATGAAATGAATAATCTCCAAATTGCTGTGGATAGGATGCACA
AAGGAGTAGCTTATGTGAAAAGAAAAATATATGAATTTATTCAACAGTCCTTCATTAGGAAACAAAAGATTTTAGATG
AAATTAAACCACTTGATGATCTAAACAACAAGAAAGACAGTTGTATGTCCAATCATACAGCAGAAATTGGGAAAGATC
TTGACTATCTTAAAGATGTAAATGGAACTACAAGTGGTATAGGAACTGGCAGCAGTGTTGAAAAATACATTATTGATG
AAAGTGATTACATGTCATTCATAAACAACCCCAGTCTTACTGTGACTGTACCAATTGCTGTAGGAGAATCTGACTTTG
AAAATTTAAACACGGAAGACTTTAGTAGTGAATCGGATCTGGAAGAAAGCAAAGAGAAACTGAATGAAAGCAGTAGCT
CATCAGAAGGTAGCACTGTGGACATCGGCGCACCTGTAGAAGAACAGCCCGTAGTGGAACCTGAAGAAACTCTTGAAC
CAGAAGCTTGTTTCACTGAAGGCTGTGTACAAAGATTCAAGTGTTGTCAAATCAATGTGGAAGAAGGCAGAGGAAAAC
AATGGTGGAACCTGAGAAGGACGTGTTTCCGAATAGTTGAACATAACTGGTTTGAGACCTTCATTGTTTTCATGATTC
TCCTTAGTAGTGGTGCTCTGGCATTTGAAGATATATATATTGATCAGCGAAAGACGATTAAGACGATGTTGGAATATG
CTGACAAGGTTTTCACTTACATTTTCATTCTGGAAATGCTTCTAAAATGGGTGGCATATGGCTATCAAACATATTTCA
CCAATGCCTGGTGTTGGCTGGACTTCTTAATTGTTGATGTTTCATTGGTCAGTTTAACAGCAAATGCCTTGGGTTACT
CAGAACTTGGAGCCATCAAATCTCTCAGGACACTAAGAGCTCTGAGACCTCTAAGAGCCTTATCTCGATTTGAAGGGA
TGAGGGTGGTTGTGAATGCCCTTTTAGGAGCAATTCCATCCATCATGAATGTGCTTCTGGTTTGTCTTATATTCTGGC
TAATTTTCAGCATCATGGGCGTAAATTTGTTTGCTGGCAAATTCTACCACTGTATTAACACCACAACTGGTGACAGGT
TTGACATCGAAGACGTGAATAATCATACTGATTGCCTAAAACTAATAGAAAGAAATGAGACTGCTCGATGGAAAAATG
TGAAAGTAAACTTTGATAATGTAGGATTTGGGTATCTCTCTTTGCTTCAAGTTGCCACATTCAAAGGATGGATGGATA
TAATGTATGCAGCAGTTGATTCCAGAAATGTGGAACTCCAGCCTAAGTATGAAGAAAGTCTGTACATGTATCTTTACT
TTGTTATTTTCATCATCTTTGGGTCCTTCTTCACCTTGAACCTGTTTATTGGTGTCATCATAGATAATTTCAACCAGC
AGAAAAAGAAGTTTGGAGGTCAAGACATCTTTATGACAGAAGAACAGAAGAAATACTATAATGCAATGAAAAAATTAG
GATCGAAAAAACCGCAAAAGCCTATACCTCGACCAGGAAACAAATTTCAAGGAATGGTCTTTGACTTCGTAACCAGAC
AAGTTTTTGACATAAGCATCATGATTCTCATCTGTCTTAACATGGTCACAATGATGGTGGAAACAGATGACCAGAGTG
AATATGTGACTACCATTTTGTCACGCATCAATCTGGTGTTCATTGTGCTATTTACTGGAGAGTGTGTACTGAAACTCA
TCTCTCTACGCCATTATTATTTTACCATTGGATGGAATATTTTTGATTTTGTGGTTGTCATTCTCTCCATTGTAGGTA
TGTTTCTTGCCGAGCTGATAGAAAAGTATTTCGTGTCCCCTACCCTGTTCCGAGTGATCCGTCTTGCTAGGATTGGCC
GAATCCTACGTCTGATCAAAGGAGCAAAGGGGATCCGCACGCTGCTCTTTGCTTTGATGATGTCCCTTCCTGCGTTGT
TTAACATCGGCCTCCTACTCTTCCTAGTCATGTTCATCTACGCCATCTTTGGGATGTCCAACTTTGCCTATGTTAAGA
GGGAAGTTGGGATCGATGACATGTTCAACTTTGAGACCTTTGGCAACAGCATGATCTGCCTATTCCAAATTACAACCT
CTGCTGGCTGGGATGGATTGCTAGCACCCATTCTCAACAGTAAGCCACCCGACTGTGACCCTAATAAAGTTAACCCTG
GAAGCTCAGTTAAGGGAGACTGTGGGAACCCATCTGTTGGAATTTTCTTTTTTGTCAGTTACATCATCATATCCTTCC
TGGTTGTGGTGAACATGTACATCGCGGTCATCCTGGAGAACTTCAGTGTTGCTACTGAAGAAAGTGCAGAGCCTCTGA
GTGAGGATGACTTTGAGATGTTCTATGAGGTTTGGGAGAAGTTTGATCCCGATGCAACTCAGTTCATGGAATTTGAAA
AATTATCTCAGTTTGCAGCTGCGCTTGAACCGCCTCTCAATCTGCCACAACCAAACAAACTCCAGCTCATTGCCATGG
ATTTGCCCATGGTGAGTGGTGACCGGATCCACTGTCTTGATATCTTATTTGCTTTTACAAAGCGGGTTCTAGGAGAGA
GTGGAGAGATGGATGCTCTACGAATACAGATGGAAGAGCGATTCATGGCTTCCAATCCTTCCAAGGTCTCCTATCAGC
CAATCACTACTACTTTAAAACGAAAACAAGAGGAAGTATCTGCTGTCATTATTCAGCGTGCTTACAGACGCCACCTTT
TAAAGCGAACTGTAAAACAAGCTTCCTTTACGTACAATAAAAACAAAATCAAAGGTGGGGCTAATCTTCTTATAAAAG
AAGACATGATAATTGACAGAATAAATGAAAACTCTATTACAGAAAAAACTGATCTGACCATGTCCACTGCAGCTTGTC
CACCTTCCTATGACCGGGTGACAAAGCCAATTGTGGAAAAACATGAGCAAGAAGGCAAAGATGAAAAAGCCAAAGGGA
AATAAATGAAAATAAATAAAAATAATTGGGTGACAAATTGTTTACAGCCTGTGAAGGTGATGTATTTTTATCAACAGG
ACTCCTTTAGGAGGTCAATGCCAAACTGACTGTTTTTACACAAATCTCCTTAAGGTCAGTGCCTACAATAAGACAGTG
ACCCCTTGTCAGCAAACTGTGACTCTGTGTAAAGGGGAGATGACCTTGACAGGAGGTTACTGTTCTCACTACCAGCTG
ACACTGCTGAAGATAAGATGCACAATGGCTAGTCAGACTGTAGGGACCAGTTTCAAGGGGTGCAAACCTGTGATTTTG
GGGTTGTTTAACATGAAACACTTTAGTGTAGTAATTGTATCCACTGTTTGCATTTCAACTGCCACATTTGTCACATTT
TTATGGAATCTGTTAGTGGATTCATCTTTTTGTTAATCCATGTGTTTATTATATGTGACTATTTTTGTAAACGAAGTT
TCTGTTGAGAAATAGGCTAAGGACCTCTATAACAGGTATGCCACCTGGGGGGTATGGCAACCACATGGCCCTCCCAGC
TACACAAAGTCGTGGTTTGCATGAGGGCATGCTGCACTTAGAGATCATGCATGAGAAAAAGTCACAAGAAAAACAAAT
TCTTAAATTTCACCATATTTCTGGGAGGGGTAATTGGGTGATAAGTGGAGGTGCTTTGTTGATCTTGTTTTGCGAAAT
CCAGCCCCTAGACCAAGTAGATTATTTGTGGGTAGGCCAGTAAATCTTAGCAGGTGCAAACTTCATTCAAATGTTTGG
AGTCATAAATGTTATGTTTCTTTTTGTTGTATTAAAAAAAAAACCTGAATAGTGAATATTGCCCCTCACCCTCCACCG
CCAGAAGACTGAATTGACCAAAATTACTCTTTATAAATTTCTGCTTTTTCCTGCACTTTGTTTAGCCATCTTCGGCTC
TCAGCAAGGTTGACACTGTATATGTTAATGAAATGCTATTTATTATGTAAATAGTCATTTTACCCTGTGGTGCACGTT
TGAGCAAACAAATAATGACCTAAGCACAGTATTTATTGCATCAAATATGTACCACAAGAAATGTAGAGTGCAAGCTTT
ACACAGGTAATAAAATGTATTCTGTACCATTTATAGATAGTTTGGATGCTATCAATGCATGTTTATATTACCATGCTG
CTGTATCTGGTTTCTCTCACTGCTCAGAATCTCATTTATGAGAAACCATATGTCAGTGGTAAAGTCAAGGAAATTGTT
CAACAGATCTCATTTATTTAAGTCATTAAGCAATAGTTTGCAGCACTTTAACAGCTTTTTGGTTATTTTTACATTTTA
AGTGGATAACATATGGTATATAGCCAGACTGTACAGACATGTTTAAAAAAACACACTGCTTAACCTATTAAATATGTG
TTTAGAATTTTATAAGCAAATATAAATACTGTAAAAAGTCACTTTATTTTATTTTTCAGCATTATGTACATAAATATG
AAGAGGAAATTATCTTCAGGTTGATATCACAATCACTTTTCTTACTTTCTGTCCATAGTACTTTTTCATGAAAGAAAT
TTGCTAAATAAGACATGAAAACAAGACTGGGTAGTTGTAGATTTCTGCTTTTTAAATTACATTTGCTAATTTTAGATT
ATTTCACAATTTTAAGGAGCAAAATAGGTTCACGATTCATATCCAAATTATGCTTTGCAATTGGAAAAGGGTTTAAAA
TTTTATTTATATTTCTGGTAGTACCTGCACTAACTGAATTGAAGGTAGTGCTTATGTTATTTTTGTTCTTTTTTTCTG
ACTTCGGTTTATGTTTTCATTTCTTTGGAGTAATGCTGCTCTAGATTGTTCTAAATAGAATGTGGGCTTCATAATTTT
TTTTTCCACAAAAACAGAGTAGTCAACTTATATAGTCAATTACATCAGGACATTTTGTGTTTCTTACAGAAGCAAACC
ATAGGCTCCTCTTTTCCTTAAAACTACTTAGATAAACTGTATTCGTGAACTGCATGCTGGAAAATGCTACTATTATGC
TAAATAATGCTAACCAACATTTAAAATGTGCAAAACTAATAAAGATTACATTTTTTATTTTATTGTTTGCCCAGTCAC
TTTTTGTTAACAGAATATTCTAATGATATGGAGATTTTTTACATTACAAATTGGGGGAGAAGGGGAGCGCGCGCGCAC
ACACACACACACACACACACACACACACACACACACAGAGGCATACCCACGTTGACAACAAAACCTAGGGTAGATATG
TCACTGGAGGTAGGGGGTAATGACCTCCCAGAATTACAAGCAGCAGGTGTGTTCTCTGTTAGGAGGAAGAACTGGTGT
CAGAGGATAGCTAGTGATTCTAGGAGGAAGAGAAGTATGGAAGCCAGAGTGATGGTGGATGACCCCTTGAGCTATGAA
AAGAAACCCTTAAATCATCATTTAAAAATTTAGAATTGCCATGTGTGTAGGATACTGTGTTTGCTCCTCCAGAGCCAC
TCTCTCTGCTTCTGCATCATTCTGTGTGTCCCAGAAGGGTGACTTCTACACATTGCAAAAATGGGCTCTCCTACCTTT
GAGCTCCCAATTGGTTTGGCCAATGAGAAGCACCAGTGGGAAAGCACCAGAGAGAGAAGATTGACATAGGAATATTTC
TTCTCCAATTCCTTCTTTGCTGGGTTGGCACTGGACTCATTCCTCCCCGAAAAGTCATACTCCAATCAGACTGCCCCT
CATACAACTGAAGCTACTTTCTCTGGGGTCAGGTAATCACTCCTCCCCTTGCTCCTTCAGGTCTGCTGCTGCATTGAG
AGTGCTTTTGTATTCCTTGTAGCTTTCTCCTAACATTGCTGACACTTTTGTAAATGTCCCCTTCATGAAATTCTTCTA
TATGCCTCATTTCAGCATGCCATCTGTCTCCTGCCTGGCTGACACAAGGTGATTCAACAGCTCATGAAAGTCAGCAGG
AAGCAAAGATGTGCCTTGCTTCAGCTTGGGGTCTTAATCTTGCTAACTTTTGCAGATAAAGAAAAACAGTAACTGGGG
GAACCACAGTGAAGTCCAGTGCAGAATTCACAGATATCATGGAAAGGTTACTCGGGTGGTCCAGATAGTAAAATTAAC
AGTCTAAATTAATCTATCTAAATTTCTGAGGAACGAGAAGCCTTCCCTTGTCATCAGGTGAAGCCAGAAGAGGGAATA
TAGCCTCAACCAGAAAAGGGACAGTAATTAAAAGGCTTTTCCCATCCTTGTACAATGGACTGACTTTGCCTCTTCATA
ACATCACAATCCTAAAGCAACACAACAATTAATTCTGATATATTAGTAGCTGAAAAAAATTCCCATTTCCAACTAAGG
TAGGTCAGAATTATAGGATAAACCCTGCAGACTTTTTATACTACCCATCCACGCCATTACTCACTGTTACCTTTCCAA
ATACAAAGAGAAGAACTGGTAAAACATAATCATATAAATCTCCATATTCATTTTGAAATATTTGGCATGATATTTTCT
GTGCTAAAAAGTAATTATTCTTCAAAGAATGATGAGGTCATGTCAGTAAGACACAGGAACCAACTAGAAGGGGCTTCC
CACTGGCCAAATCTGGGGCAAGTTGAGCATCAAAATAAATGATAGTAAAAGATTATAATTCATTGAATAAGAATCAGC
AAATACATACTGATGTAAGTAAATAAGGAAAAGTACAAATCTGTTTCTTGCAGTTGAATGTTAATTAACAATTGTAGA
AGAAATAACGGAGTTAGAAAAATCACTATTTGGCAATCACCCTAATGACAATTGATTCATACAAGAATCATCAATGAG
TATTAAAACTCATGGGTGAAAGTTTGATGAGGAATAGGGTATTTATAGCATCTTAAAGTATCTCTTCTCTATTAAGTA
GAAAATTTAAACAGAAGAAAGTATACTTTGGAGAAATACAGCAGACAATACCTTCAAAGATATCATCAATTATGAGAC
CAACTGATACTATGTGCCTCCTGATAAGATATACTGAAAGGGCCACATTACTTCTTGGTACACAGTCAAAATTTTAAA
ACCAGAATCTAACTACAAGGAAAATCAAATTGAGGACACTCTATAAAATAAGTGGACTGAACTCCTTAAAAATGTCAA
TGTCATGAAAGACAAAGAAAGGCTAAAGAATTCCATGAGGTCAAAGAACTATGACAACTAAACACAATTCTGGATGGA
ATATCAAATTAAAAAATAACAGATAAATAATATTATTGGGAAAGTTGAATAAATTTGAATATGGACTGTTTATTAGTT
ATTAGTATTATAATAGTGTTAATTTTCCTAATTTTGTTAAGACTAGTGTGCCTGTTCCATGAAAATAGAAAATGTTCT
TATTCTCTGAAAATGCATGCTAAAGTATTTAGGGGTGAATGCAACAATGTCTGCAGCTCATTCTTGAATCAGTTCAAA
GAAAAATGAGTTACATTTATATATATATGTATGTATGTAAACAGACATAGATAAAAGTATAGATGTGTGTGTGTCTTT
AGAAAGGGGAGGATTTTTTTTTTTTTTTTGCTGTGTGTTACTGAAGTGCCTATGTCTGCGTGTTCACACTATCATATT
TTGTATGCCCTGGACTTTATAATTTCTACCTTCAAAATTAGATCTACTGTTGGTAATTAATTCAATATATACTGGTTT
TTTAACTACTATTCTCATTTCCTAGCAGTAATCTTCCTGAAAAGTCACAGAAATGATTACATTCCTTGTTCTTCATAA
TAATCACTGTTTAATTAAAATAAGAATATTTTAGAAAAGATCTGCGGCATAGTGGTTAAGACCCCAGTATTTGATGCT
AAACAGATCTGATTTGGATAACAGAAGGTGGCACTTTGCTGTTTAAGCTGGGGACCAGACACTGTGGGTATAAATAGT
AATTCCAAACACAGCTCCACAGAGCAGCACCCTTATGACAAGGTTTTCATATGTCTATAGTTAAGCCAGAAAATTAAG
AATAATGCCATAAATATTTATAAAGCTGAACATATCCAAGTTAAAGACCTTTATCCTGAAATTGTATCTTTTAGATTA
TTTTCTAAAGACTAATACCATTTAATGTTTAAATGTTCTTTGGAAATGATGGTGAGAATACGTGATAATGGGTCATTG
GTTTTAATATTTTATTTAGCCAAGTGGAAAATTGGCAACCTGGTGTCGGTCCTCCCATTTGTATTTTACTGGTGCATG
AAATCCAAAAGTCTAGTAACCATTGGGACAGACAACTCTACTGCATAAGTTTGTATGTTTGTATATCTGTATCACAAA
GCCCAGACACTCGAACTATATAAACTTGTCGCACTAAAGACAGCAAATATGTCTGGTAATTGCATATTCTTCATGTGT
GCACTGGAATTTCTTATTATATAAGAAAATAAATGTGTTTCTAAACCACCATGAATTGGGTACTGCTGATAGTACTAT
TCTTCCTGGCACATGGAAATATCCCACTGAGGTTGTCAATCAATATTGATTTAATCAGTGTGGCAGCAAAGGCACTAG
AGGAGGAAAATACTCTAGACAATCAAGTATAGATGGGAAAATTGCATCACCAAGAAATTCTTAAAATCACAAGTGGAT
AATTTGCAATATCAATTGATTTTACTCAACTGCTTGAGTCCTTTATCATTCCGTTAGAACACTCCTCATATTTGCAGA
TAACTCAGCCCCTCACCTCAGCAACAAGCAAGAGGACATCATTATAAACTTCCTCAATTTCTTTCCTATCCAGTGAAA
AATATCTAAGTCCCTTTTCCCTTCCCTTTTCCTTCCCTTCCCTTCCTTTTTCTTTCCTTCCATTCTCTTCCTTTTTCC
TTCCTTTTTCTTTCCTTCCATTCTCTTCCTTTTTCCTTCCTTTTTCTTACTTTACCTCCCCCTCCTACCTTTCCCTTT
CTCCCTTCCTCCCTCTCTCCCTTCCTTCCTCTCTCCCTCTCCTTCCTTCCTTCCTTCAATCCATACTTTTATATCTTT
TCAGAGATCAATTTTTCCCATTCATTTCTACATTCTCAGATACCTTGCTCCATGACTTGTTCTACTTCTCTTCTATCT
TTATTCTTTCCCTCTTGACTGATCCTTTTTGTCTGCCTTTGCATTTTCAAAATCAGTTTTTCTAATCTTAAAAAATAA
CTTTGCTCAACCCTGATGCATCTTTATTCACTGTGTCATTATCTCATTATTTTTAAAGGTTAGTTTCTAAAGCCAGGG
CTCTATACTCACTACCTTCACTCTTGGTTAAAATTAAAAGAAATATAGGTCCCAGTGATATTTATGGGAAAATTTGAC
TGTGAATAATCTTGTTGATATAAACACTTTGGGTTTTCAAAATGTTATGCTAGCTGTTAACTTTCCCAGTAACATTAT
GAGGGAAGGGCTGGAAAATACAAAAGGTAGACATAGGAACCGAGAACACCACCCCTATGGAGCCAAGCTATACATACT
CTGTCTCTGGTTATCATAAATACATCTCCTATATTCAATGCTTGCTTAGAAAGGCATGATGTCCACGGAGCAATTCAA
TTGTTCACTGTGATGGGTTTTTTTGTTTTCTCCTCATGGAATGCAACTTTTATTTAT
SEQ ID NO: 10 >NM_001353951.2 SCN1A [organism = Homo sapiens] [GeneID = 6323]
[transcript = 8]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGAATTTCATATGCAGAATAAATGGTAATTAAAATGTGCAGGATGACAAGATGGAGCAAACAG
TGCTTGTACCACCAGGACCTGACAGCTTCAACTTCTTCACCAGAGAATCTCTTGCGGCTATTGAAAGACGCATTGCAG
AAGAAAAGGCAAAGAATCCCAAACCAGACAAAAAAGATGACGACGAAAATGGCCCAAAGCCAAATAGTGACTTGGAAG
CTGGAAAGAACCTTCCATTTATTTATGGAGACATTCCTCCAGAGATGGTGTCAGAGCCCCTGGAGGACCTGGACCCCT
ACTATATCAATAAGAAAACTTTTATAGTATTGAATAAAGGGAAGGCCATCTTCCGGTTCAGTGCCACCTCTGCCCTGT
ACATTTTAACTCCCTTCAATCCTCTTAGGAAAATAGCTATTAAGATTTTGGTACATTCATTATTCAGCATGCTAATTA
TGTGCACTATTTTGACAAACTGTGTGTTTATGACAATGAGTAACCCTCCTGATTGGACAAAGAATGTAGAATACACCT
TCACAGGAATATATACTTTTGAATCACTTATAAAAATTATTGCAAGGGGATTCTGTTTAGAAGATTTTACTTTCCTTC
GGGATCCATGGAACTGGCTCGATTTCACTGTCATTACATTTGCGTACGTCACAGAGTTTGTGGACCTGGGCAATGTCT
CGGCATTGAGAACATTCAGAGTTCTCCGAGCATTGAAGACGATTTCAGTCATTCCAGGCCTGAAAACCATTGTGGGAG
CCCTGATCCAGTCTGTGAAGAAGCTCTCAGATGTAATGATCCTGACTGTGTTCTGTCTGAGCGTATTTGCTCTAATTG
GGCTGCAGCTGTTCATGGGCAACCTGAGGAATAAATGTATACAATGGCCTCCCACCAATGCTTCCTTGGAGGAACATA
GTATAGAAAAGAATATAACTGTGAATTATAATGGTACACTTATAAATGAAACTGTCTTTGAGTTTGACTGGAAGTCAT
ATATTCAAGATTCAAGATATCATTATTTCCTGGAGGGTTTTTTAGATGCACTACTATGTGGAAATAGCTCTGATGCAG
GCCAATGTCCAGAGGGATATATGTGTGTGAAAGCTGGTAGAAATCCCAATTATGGCTACACAAGCTTTGATACCTTCA
GTTGGGCTTTTTTGTCCTTGTTTCGACTAATGACTCAGGACTTCTGGGAAAATCTTTATCAACTGACATTACGTGCTG
CTGGGAAAACGTACATGATATTTTTTGTATTGGTCATTTTCTTGGGCTCATTCTACCTAATAAATTTGATCCTGGCTG
TGGTGGCCATGGCCTACGAGGAACAGAATCAGGCCACCTTGGAAGAAGCAGAACAGAAAGAGGCCGAATTTCAGCAGA
TGATTGAACAGCTTAAAAAGCAACAGGAGGCAGCTCAGCAGGCAGCAACGGCAACTGCCTCAGAACATTCCAGAGAGC
CCAGTGCAGCAGGCAGGCTCTCAGACAGCTCATCTGAAGCCTCTAAGTTGAGTTCCAAGAGTGCTAAGGAAAGAAGAA
ATCGGAGGAAGAAAAGAAAACAGAAAGAGCAGTCTGGTGGGGAAGAGAAAGATGAGGATGAATTCCAAAAATCTGAAT
CTGAGGACAGCATCAGGAGGAAAGGTTTTCGCTTCTCCATTGAAGGGAACCGATTGACATATGAAAAGAGGTACTCCT
CCCCACACCAGTCTTTGTTGAGCATCCGTGGCTCCCTATTTTCACCAAGGCGAAATAGCAGAACAAGCCTTTTCAGCT
TTAGAGGGCGAGCAAAGGATGTGGGATCTGAGAACGACTTCGCAGATGATGAGCACAGCACCTTTGAGGATAACGAGA
GCCGTAGAGATTCCTTGTTTGTGCCCCGACGACACGGAGAGAGACGCAACAGCAACCTGAGTCAGACCAGTAGGTCAT
CCCGGATGCTGGCAGTGTTTCCAGCGAATGGGAAGATGCACAGCACTGTGGATTGCAATGGTGTGGTTTCCTTGGTTG
GTGGACCTTCAGTTCCTACATCGCCTGTTGGACAGCTTCTGCCAGAGGGAACAACCACTGAAACTGAAATGAGAAAGA
GAAGGTCAAGTTCTTTCCACGTTTCCATGGACTTTCTAGAAGATCCTTCCCAAAGGCAACGAGCAATGAGTATAGCCA
GCATTCTAACAAATACAGTAGAAGAACTTGAAGAATCCAGGCAGAAATGCCCACCCTGTTGGTATAAATTTTCCAACA
TATTCTTAATCTGGGACTGTTCTCCATATTGGTTAAAAGTGAAACATGTTGTCAACCTGGTTGTGATGGACCCATTTG
TTGACCTGGCCATCACCATCTGTATTGTCTTAAATACTCTTTTCATGGCCATGGAGCACTATCCAATGACGGACCATT
TCAATAATGTGCTTACAGTAGGAAACTTGGTTTTCACTGGGATCTTTACAGCAGAAATGTTTCTGAAAATTATTGCCA
TGGATCCTTACTATTATTTCCAAGAAGGCTGGAATATCTTTGACGGTTTTATTGTGACGCTTAGCCTGGTAGAACTTG
GACTCGCCAATGTGGAAGGATTATCTGTTCTCCGTTCATTTCGATTGCTGCGAGTTTTCAAGTTGGCAAAATCTTGGC
CAACGTTAAATATGCTAATAAAGATCATCGGCAATTCCGTGGGGGCTCTGGGAAATTTAACCCTCGTCTTGGCCATCA
TCGTCTTCATTTTTGCCGTGGTCGGCATGCAGCTCTTTGGTAAAAGCTACAAAGATTGTGTCTGCAAGATCGCCAGTG
ATTGTCAACTCCCACGCTGGCACATGAATGACTTCTTCCACTCCTTCCTGATTGTGTTCCGCGTGCTGTGTGGGGAGT
GGATAGAGACCATGTGGGACTGTATGGAGGTTGCTGGTCAAGCCATGTGCCTTACTGTCTTCATGATGGTCATGGTGA
TTGGAAACCTAGTGGTCCTGAATCTCTTTCTGGCCTTGCTTCTGAGCTCATTTAGTGCAGACAACCTTGCAGCCACTG
ATGATGATAATGAAATGAATAATCTCCAAATTGCTGTGGATAGGATGCACAAAGGAGTAGCTTATGTGAAAAGAAAAA
TATATGAATTTATTCAACAGTCCTTCATTAGGAAACAAAAGATTTTAGATGAAATTAAACCACTTGATGATCTAAACA
ACAAGAAAGACAGTTGTATGTCCAATCATACAGCAGAAATTGGGAAAGATCTTGACTATCTTAAAGATGTAAATGGAA
CTACAAGTGGTATAGGAACTGGCAGCAGTGTTGAAAAATACATTATTGATGAAAGTGATTACATGTCATTCATAAACA
ACCCCAGTCTTACTGTGACTGTACCAATTGCTGTAGGAGAATCTGACTTTGAAAATTTAAACACGGAAGACTTTAGTA
GTGAATCGGATCTGGAAGAAAGCAAAGAGAAACTGAATGAAAGCAGTAGCTCATCAGAAGGTAGCACTGTGGACATCG
GCGCACCTGTAGAAGAACAGCCCGTAGTGGAACCTGAAGAAACTCTTGAACCAGAAGCTTGTTTCACTGAAGGCTGTG
TACAAAGATTCAAGTGTTGTCAAATCAATGTGGAAGAAGGCAGAGGAAAACAATGGTGGAACCTGAGAAGGACGTGTT
TCCGAATAGTTGAACATAACTGGTTTGAGACCTTCATTGTTTTCATGATTCTCCTTAGTAGTGGTGCTCTGGCATTTG
AAGATATATATATTGATCAGCGAAAGACGATTAAGACGATGTTGGAATATGCTGACAAGGTTTTCACTTACATTTTCA
TTCTGGAAATGCTTCTAAAATGGGTGGCATATGGCTATCAAACATATTTCACCAATGCCTGGTGTTGGCTGGACTTCT
TAATTGTTGATGTTTCATTGGTCAGTTTAACAGCAAATGCCTTGGGTTACTCAGAACTTGGAGCCATCAAATCTCTCA
GGACACTAAGAGCTCTGAGACCTCTAAGAGCCTTATCTCGATTTGAAGGGATGAGGGTGGTTGTGAATGCCCTTTTAG
GAGCAATTCCATCCATCATGAATGTGCTTCTGGTTTGTCTTATATTCTGGCTAATTTTCAGCATCATGGGCGTAAATT
TGTTTGCTGGCAAATTCTACCACTGTATTAACACCACAACTGGTGACAGGTTTGACATCGAAGACGTGAATAATCATA
CTGATTGCCTAAAACTAATAGAAAGAAATGAGACTGCTCGATGGAAAAATGTGAAAGTAAACTTTGATAATGTAGGAT
TTGGGTATCTCTCTTTGCTTCAAGTTGCCACATTCAAAGGATGGATGGATATAATGTATGCAGCAGTTGATTCCAGAA
ATGTGGAACTCCAGCCTAAGTATGAAGAAAGTCTGTACATGTATCTTTACTTTGTTATTTTCATCATCTTTGGGTCCT
TCTTCACCTTGAACCTGTTTATTGGTGTCATCATAGATAATTTCAACCAGCAGAAAAAGAAGTTTGGAGGTCAAGACA
TCTTTATGACAGAAGAACAGAAGAAATACTATAATGCAATGAAAAAATTAGGATCGAAAAAACCGCAAAAGCCTATAC
CTCGACCAGGAAACAAATTTCAAGGAATGGTCTTTGACTTCGTAACCAGACAAGTTTTTGACATAAGCATCATGATTC
TCATCTGTCTTAACATGGTCACAATGATGGTGGAAACAGATGACCAGAGTGAATATGTGACTACCATTTTGTCACGCA
TCAATCTGGTGTTCATTGTGCTATTTACTGGAGAGTGTGTACTGAAACTCATCTCTCTACGCCATTATTATTTTACCA
TTGGATGGAATATTTTTGATTTTGTGGTTGTCATTCTCTCCATTGTAGGTATGTTTCTTGCCGAGCTGATAGAAAAGT
ATTTCGTGTCCCCTACCCTGTTCCGAGTGATCCGTCTTGCTAGGATTGGCCGAATCCTACGTCTGATCAAAGGAGCAA
AGGGGATCCGCACGCTGCTCTTTGCTTTGATGATGTCCCTTCCTGCGTTGTTTAACATCGGCCTCCTACTCTTCCTAG
TCATGTTCATCTACGCCATCTTTGGGATGTCCAACTTTGCCTATGTTAAGAGGGAAGTTGGGATCGATGACATGTTCA
ACTTTGAGACCTTTGGCAACAGCATGATCTGCCTATTCCAAATTACAACCTCTGCTGGCTGGGATGGATTGCTAGCAC
CCATTCTCAACAGTAAGCCACCCGACTGTGACCCTAATAAAGTTAACCCTGGAAGCTCAGTTAAGGGAGACTGTGGGA
ACCCATCTGTTGGAATTTTCTTTTTTGTCAGTTACATCATCATATCCTTCCTGGTTGTGGTGAACATGTACATCGCGG
TCATCCTGGAGAACTTCAGTGTTGCTACTGAAGAAAGTGCAGAGCCTCTGAGTGAGGATGACTTTGAGATGTTCTATG
AGGTTTGGGAGAAGTTTGATCCCGATGCAACTCAGTTCATGGAATTTGAAAAATTATCTCAGTTTGCAGCTGCGCTTG
AACCGCCTCTCAATCTGCCACAACCAAACAAACTCCAGCTCATTGCCATGGATTTGCCCATGGTGAGTGGTGACCGGA
TCCACTGTCTTGATATCTTATTTGCTTTTACAAAGCGGGTTCTAGGAGAGAGTGGAGAGATGGATGCTCTACGAATAC
AGATGGAAGAGCGATTCATGGCTTCCAATCCTTCCAAGGTCTCCTATCAGCCAATCACTACTACTTTAAAACGAAAAC
AAGAGGAAGTATCTGCTGTCATTATTCAGCGTGCTTACAGACGCCACCTTTTAAAGCGAACTGTAAAACAAGCTTCCT
TTACGTACAATAAAAACAAAATCAAAGGTGGGGCTAATCTTCTTATAAAAGAAGACATGATAATTGACAGAATAAATG
AAAACTCTATTACAGAAAAAACTGATCTGACCATGTCCACTGCAGCTTGTCCACCTTCCTATGACCGGGTGACAAAGC
CAATTGTGGAAAAACATGAGCAAGAAGGCAAAGATGAAAAAGCCAAAGGGAAATAAATGAAAATAAATAAAAATAATT
GGGTGACAAATTGTTTACAGCCTGTGAAGGTGATGTATTTTTATCAACAGGACTCCTTTAGGAGGTCAATGCCAAACT
GACTGTTTTTACACAAATCTCCTTAAGGTCAGTGCCTACAATAAGACAGTGACCCCTTGTCAGCAAACTGTGACTCTG
TGTAAAGGGGAGATGACCTTGACAGGAGGTTACTGTTCTCACTACCAGCTGACACTGCTGAAGATAAGATGCACAATG
GCTAGTCAGACTGTAGGGACCAGTTTCAAGGGGTGCAAACCTGTGATTTTGGGGTTGTTTAACATGAAACACTTTAGT
GTAGTAATTGTATCCACTGTTTGCATTTCAACTGCCACATTTGTCACATTTTTATGGAATCTGTTAGTGGATTCATCT
TTTTGTTAATCCATGTGTTTATTATATGTGACTATTTTTGTAAACGAAGTTTCTGTTGAGAAATAGGCTAAGGACCTC
TATAACAGGTATGCCACCTGGGGGGTATGGCAACCACATGGCCCTCCCAGCTACACAAAGTCGTGGTTTGCATGAGGG
CATGCTGCACTTAGAGATCATGCATGAGAAAAAGTCACAAGAAAAACAAATTCTTAAATTTCACCATATTTCTGGGAG
GGGTAATTGGGTGATAAGTGGAGGTGCTTTGTTGATCTTGTTTTGCGAAATCCAGCCCCTAGACCAAGTAGATTATTT
GTGGGTAGGCCAGTAAATCTTAGCAGGTGCAAACTTCATTCAAATGTTTGGAGTCATAAATGTTATGTTTCTTTTTGT
TGTATTAAAAAAAAAACCTGAATAGTGAATATTGCCCCTCACCCTCCACCGCCAGAAGACTGAATTGACCAAAATTAC
TCTTTATAAATTTCTGCTTTTTCCTGCACTTTGTTTAGCCATCTTCGGCTCTCAGCAAGGTTGACACTGTATATGTTA
ATGAAATGCTATTTATTATGTAAATAGTCATTTTACCCTGTGGTGCACGTTTGAGCAAACAAATAATGACCTAAGCAC
AGTATTTATTGCATCAAATATGTACCACAAGAAATGTAGAGTGCAAGCTTTACACAGGTAATAAAATGTATTCTGTAC
CATTTATAGATAGTTTGGATGCTATCAATGCATGTTTATATTACCATGCTGCTGTATCTGGTTTCTCTCACTGCTCAG
AATCTCATTTATGAGAAACCATATGTCAGTGGTAAAGTCAAGGAAATTGTTCAACAGATCTCATTTATTTAAGTCATT
AAGCAATAGTTTGCAGCACTTTAACAGCTTTTTGGTTATTTTTACATTTTAAGTGGATAACATATGGTATATAGCCAG
ACTGTACAGACATGTTTAAAAAAACACACTGCTTAACCTATTAAATATGTGTTTAGAATTTTATAAGCAAATATAAAT
ACTGTAAAAAGTCACTTTATTTTATTTTTCAGCATTATGTACATAAATATGAAGAGGAAATTATCTTCAGGTTGATAT
CACAATCACTTTTCTTACTTTCTGTCCATAGTACTTTTTCATGAAAGAAATTTGCTAAATAAGACATGAAAACAAGAC
TGGGTAGTTGTAGATTTCTGCTTTTTAAATTACATTTGCTAATTTTAGATTATTTCACAATTTTAAGGAGCAAAATAG
GTTCACGATTCATATCCAAATTATGCTTTGCAATTGGAAAAGGGTTTAAAATTTTATTTATATTTCTGGTAGTACCTG
CACTAACTGAATTGAAGGTAGTGCTTATGTTATTTTTGTTCTTTTTTTCTGACTTCGGTTTATGTTTTCATTTCTTTG
GAGTAATGCTGCTCTAGATTGTTCTAAATAGAATGTGGGCTTCATAATTTTTTTTTCCACAAAAACAGAGTAGTCAAC
TTATATAGTCAATTACATCAGGACATTTTGTGTTTCTTACAGAAGCAAACCATAGGCTCCTCTTTTCCTTAAAACTAC
TTAGATAAACTGTATTCGTGAACTGCATGCTGGAAAATGCTACTATTATGCTAAATAATGCTAACCAACATTTAAAAT
GTGCAAAACTAATAAAGATTACATTTTTTATTTTATTGTTTGCCCAGTCACTTTTTGTTAACAGAATATTCTAATGAT
ATGGAGATTTTTTACATTACAAATTGGGGGAGAAGGGGAGCGCGCGCGCACACACACACACACACACACACACACACA
CACACACACAGAGGCATACCCACGTTGACAACAAAACCTAGGGTAGATATGTCACTGGAGGTAGGGGGTAATGACCTC
CCAGAATTACAAGCAGCAGGTGTGTTCTCTGTTAGGAGGAAGAACTGGTGTCAGAGGATAGCTAGTGATTCTAGGAGG
AAGAGAAGTATGGAAGCCAGAGTGATGGTGGATGACCCCTTGAGCTATGAAAAGAAACCCTTAAATCATCATTTAAAA
ATTTAGAATTGCCATGTGTGTAGGATACTGTGTTTGCTCCTCCAGAGCCACTCTCTCTGCTTCTGCATCATTCTGTGT
GTCCCAGAAGGGTGACTTCTACACATTGCAAAAATGGGCTCTCCTACCTTTGAGCTCCCAATTGGTTTGGCCAATGAG
AAGCACCAGTGGGAAAGCACCAGAGAGAGAAGATTGACATAGGAATATTTCTTCTCCAATTCCTTCTTTGCTGGGTTG
GCACTGGACTCATTCCTCCCCGAAAAGTCATACTCCAATCAGACTGCCCCTCATACAACTGAAGCTACTTTCTCTGGG
GTCAGGTAATCACTCCTCCCCTTGCTCCTTCAGGTCTGCTGCTGCATTGAGAGTGCTTTTGTATTCCTTGTAGCTTTC
TCCTAACATTGCTGACACTTTTGTAAATGTCCCCTTCATGAAATTCTTCTATATGCCTCATTTCAGCATGCCATCTGT
CTCCTGCCTGGCTGACACAAGGTGATTCAACAGCTCATGAAAGTCAGCAGGAAGCAAAGATGTGCCTTGCTTCAGCTT
GGGGTCTTAATCTTGCTAACTTTTGCAGATAAAGAAAAACAGTAACTGGGGGAACCACAGTGAAGTCCAGTGCAGAAT
TCACAGATATCATGGAAAGGTTACTCGGGTGGTCCAGATAGTAAAATTAACAGTCTAAATTAATCTATCTAAATTTCT
GAGGAACGAGAAGCCTTCCCTTGTCATCAGGTGAAGCCAGAAGAGGGAATATAGCCTCAACCAGAAAAGGGACAGTAA
TTAAAAGGCTTTTCCCATCCTTGTACAATGGACTGACTTTGCCTCTTCATAACATCACAATCCTAAAGCAACACAACA
ATTAATTCTGATATATTAGTAGCTGAAAAAAATTCCCATTTCCAACTAAGGTAGGTCAGAATTATAGGATAAACCCTG
CAGACTTTTTATACTACCCATCCACGCCATTACTCACTGTTACCTTTCCAAATACAAAGAGAAGAACTGGTAAAACAT
AATCATATAAATCTCCATATTCATTTTGAAATATTTGGCATGATATTTTCTGTGCTAAAAAGTAATTATTCTTCAAAG
AATGATGAGGTCATGTCAGTAAGACACAGGAACCAACTAGAAGGGGCTTCCCACTGGCCAAATCTGGGGCAAGTTGAG
CATCAAAATAAATGATAGTAAAAGATTATAATTCATTGAATAAGAATCAGCAAATACATACTGATGTAAGTAAATAAG
GAAAAGTACAAATCTGTTTCTTGCAGTTGAATGTTAATTAACAATTGTAGAAGAAATAACGGAGTTAGAAAAATCACT
ATTTGGCAATCACCCTAATGACAATTGATTCATACAAGAATCATCAATGAGTATTAAAACTCATGGGTGAAAGTTTGA
TGAGGAATAGGGTATTTATAGCATCTTAAAGTATCTCTTCTCTATTAAGTAGAAAATTTAAACAGAAGAAAGTATACT
TTGGAGAAATACAGCAGACAATACCTTCAAAGATATCATCAATTATGAGACCAACTGATACTATGTGCCTCCTGATAA
GATATACTGAAAGGGCCACATTACTTCTTGGTACACAGTCAAAATTTTAAAACCAGAATCTAACTACAAGGAAAATCA
AATTGAGGACACTCTATAAAATAAGTGGACTGAACTCCTTAAAAATGTCAATGTCATGAAAGACAAAGAAAGGCTAAA
GAATTCCATGAGGTCAAAGAACTATGACAACTAAACACAATTCTGGATGGAATATCAAATTAAAAAATAACAGATAAA
TAATATTATTGGGAAAGTTGAATAAATTTGAATATGGACTGTTTATTAGTTATTAGTATTATAATAGTGTTAATTTTC
CTAATTTTGTTAAGACTAGTGTGCCTGTTCCATGAAAATAGAAAATGTTCTTATTCTCTGAAAATGCATGCTAAAGTA
TTTAGGGGTGAATGCAACAATGTCTGCAGCTCATTCTTGAATCAGTTCAAAGAAAAATGAGTTACATTTATATATATA
TGTATGTATGTAAACAGACATAGATAAAAGTATAGATGTGTGTGTGTCTTTAGAAAGGGGAGGATTTTTTTTTTTTTT
TTGCTGTGTGTTACTGAAGTGCCTATGTCTGCGTGTTCACACTATCATATTTTGTATGCCCTGGACTTTATAATTTCT
ACCTTCAAAATTAGATCTACTGTTGGTAATTAATTCAATATATACTGGTTTTTTAACTACTATTCTCATTTCCTAGCA
GTAATCTTCCTGAAAAGTCACAGAAATGATTACATTCCTTGTTCTTCATAATAATCACTGTTTAATTAAAATAAGAAT
ATTTTAGAAAAGATCTGCGGCATAGTGGTTAAGACCCCAGTATTTGATGCTAAACAGATCTGATTTGGATAACAGAAG
GTGGCACTTTGCTGTTTAAGCTGGGGACCAGACACTGTGGGTATAAATAGTAATTCCAAACACAGCTCCACAGAGCAG
CACCCTTATGACAAGGTTTTCATATGTCTATAGTTAAGCCAGAAAATTAAGAATAATGCCATAAATATTTATAAAGCT
GAACATATCCAAGTTAAAGACCTTTATCCTGAAATTGTATCTTTTAGATTATTTTCTAAAGACTAATACCATTTAATG
TTTAAATGTTCTTTGGAAATGATGGTGAGAATACGTGATAATGGGTCATTGGTTTTAATATTTTATTTAGCCAAGTGG
AAAATTGGCAACCTGGTGTCGGTCCTCCCATTTGTATTTTACTGGTGCATGAAATCCAAAAGTCTAGTAACCATTGGG
ACAGACAACTCTACTGCATAAGTTTGTATGTTTGTATATCTGTATCACAAAGCCCAGACACTCGAACTATATAAACTT
GTCGCACTAAAGACAGCAAATATGTCTGGTAATTGCATATTCTTCATGTGTGCACTGGAATTTCTTATTATATAAGAA
AATAAATGTGTTTCTAAACCACCATGAATTGGGTACTGCTGATAGTACTATTCTTCCTGGCACATGGAAATATCCCAC
TGAGGTTGTCAATCAATATTGATTTAATCAGTGTGGCAGCAAAGGCACTAGAGGAGGAAAATACTCTAGACAATCAAG
TATAGATGGGAAAATTGCATCACCAAGAAATTCTTAAAATCACAAGTGGATAATTTGCAATATCAATTGATTTTACTC
AACTGCTTGAGTCCTTTATCATTCCGTTAGAACACTCCTCATATTTGCAGATAACTCAGCCCCTCACCTCAGCAACAA
GCAAGAGGACATCATTATAAACTTCCTCAATTTCTTTCCTATCCAGTGAAAAATATCTAAGTCCCTTTTCCCTTCCCT
TTTCCTTCCCTTCCCTTCCTTTTTCTTTCCTTCCATTCTCTTCCTTTTTCCTTCCTTTTTCTTTCCTTCCATTCTCTT
CCTTTTTCCTTCCTTTTTCTTACTTTACCTCCCCCTCCTACCTTTCCCTTTCTCCCTTCCTCCCTCTCTCCCTTCCTT
CCTCTCTCCCTCTCCTTCCTTCCTTCCTTCAATCCATACTTTTATATCTTTTCAGAGATCAATTTTTCCCATTCATTT
CTACATTCTCAGATACCTTGCTCCATGACTTGTTCTACTTCTCTTCTATCTTTATTCTTTCCCTCTTGACTGATCCTT
TTTGTCTGCCTTTGCATTTTCAAAATCAGTTTTTCTAATCTTAAAAAATAACTTTGCTCAACCCTGATGCATCTTTAT
TCACTGTGTCATTATCTCATTATTTTTAAAGGTTAGTTTCTAAAGCCAGGGCTCTATACTCACTACCTTCACTCTTGG
TTAAAATTAAAAGAAATATAGGTCCCAGTGATATTTATGGGAAAATTTGACTGTGAATAATCTTGTTGATATAAACAC
TTTGGGTTTTCAAAATGTTATGCTAGCTGTTAACTTTCCCAGTAACATTATGAGGGAAGGGCTGGAAAATACAAAAGG
TAGACATAGGAACCGAGAACACCACCCCTATGGAGCCAAGCTATACATACTCTGTCTCTGGTTATCATAAATACATCT
CCTATATTCAATGCTTGCTTAGAAAGGCATGATGTCCACGGAGCAATTCAATTGTTCACTGTGATGGGTTTTTTTGTT
TTCTCCTCATGGAATGCAACTTTTATTTAT
SEQ ID NO: 11 >NM_001353948.2 SCN1A [organism = Homo sapiens] [GeneID = 6323]
[transcript = 5]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGAATTTCATATGCAGAATAAATGGTAATTAAAATGTGCAGGATGACAAGATGGAGCAAACAG
TGCTTGTACCACCAGGACCTGACAGCTTCAACTTCTTCACCAGAGAATCTCTTGCGGCTATTGAAAGACGCATTGCAG
AAGAAAAGGCAAAGAATCCCAAACCAGACAAAAAAGATGACGACGAAAATGGCCCAAAGCCAAATAGTGACTTGGAAG
CTGGAAAGAACCTTCCATTTATTTATGGAGACATTCCTCCAGAGATGGTGTCAGAGCCCCTGGAGGACCTGGACCCCT
ACTATATCAATAAGAAAACTTTTATAGTATTGAATAAAGGGAAGGCCATCTTCCGGTTCAGTGCCACCTCTGCCCTGT
ACATTTTAACTCCCTTCAATCCTCTTAGGAAAATAGCTATTAAGATTTTGGTACATTCATTATTCAGCATGCTAATTA
TGTGCACTATTTTGACAAACTGTGTGTTTATGACAATGAGTAACCCTCCTGATTGGACAAAGAATGTAGAATACACCT
TCACAGGAATATATACTTTTGAATCACTTATAAAAATTATTGCAAGGGGATTCTGTTTAGAAGATTTTACTTTCCTTC
GGGATCCATGGAACTGGCTCGATTTCACTGTCATTACATTTGCGTACGTCACAGAGTTTGTGGACCTGGGCAATGTCT
CGGCATTGAGAACATTCAGAGTTCTCCGAGCATTGAAGACGATTTCAGTCATTCCAGGCCTGAAAACCATTGTGGGAG
CCCTGATCCAGTCTGTGAAGAAGCTCTCAGATGTAATGATCCTGACTGTGTTCTGTCTGAGCGTATTTGCTCTAATTG
GGCTGCAGCTGTTCATGGGCAACCTGAGGAATAAATGTATACAATGGCCTCCCACCAATGCTTCCTTGGAGGAACATA
GTATAGAAAAGAATATAACTGTGAATTATAATGGTACACTTATAAATGAAACTGTCTTTGAGTTTGACTGGAAGTCAT
ATATTCAAGATTCAAGATATCATTATTTCCTGGAGGGTTTTTTAGATGCACTACTATGTGGAAATAGCTCTGATGCAG
GCCAATGTCCAGAGGGATATATGTGTGTGAAAGCTGGTAGAAATCCCAATTATGGCTACACAAGCTTTGATACCTTCA
GTTGGGCTTTTTTGTCCTTGTTTCGACTAATGACTCAGGACTTCTGGGAAAATCTTTATCAACTGACATTACGTGCTG
CTGGGAAAACGTACATGATATTTTTTGTATTGGTCATTTTCTTGGGCTCATTCTACCTAATAAATTTGATCCTGGCTG
TGGTGGCCATGGCCTACGAGGAACAGAATCAGGCCACCTTGGAAGAAGCAGAACAGAAAGAGGCCGAATTTCAGCAGA
TGATTGAACAGCTTAAAAAGCAACAGGAGGCAGCTCAGCAGGCAGCAACGGCAACTGCCTCAGAACATTCCAGAGAGC
CCAGTGCAGCAGGCAGGCTCTCAGACAGCTCATCTGAAGCCTCTAAGTTGAGTTCCAAGAGTGCTAAGGAAAGAAGAA
ATCGGAGGAAGAAAAGAAAACAGAAAGAGCAGTCTGGTGGGGAAGAGAAAGATGAGGATGAATTCCAAAAATCTGAAT
CTGAGGACAGCATCAGGAGGAAAGGTTTTCGCTTCTCCATTGAAGGGAACCGATTGACATATGAAAAGAGGTACTCCT
CCCCACACCAGTCTTTGTTGAGCATCCGTGGCTCCCTATTTTCACCAAGGCGAAATAGCAGAACAAGCCTTTTCAGCT
TTAGAGGGCGAGCAAAGGATGTGGGATCTGAGAACGACTTCGCAGATGATGAGCACAGCACCTTTGAGGATAACGAGA
GCCGTAGAGATTCCTTGTTTGTGCCCCGACGACACGGAGAGAGACGCAACAGCAACCTGAGTCAGACCAGTAGGTCAT
CCCGGATGCTGGCAGTGTTTCCAGCGAATGGGAAGATGCACAGCACTGTGGATTGCAATGGTGTGGTTTCCTTGGTTG
GTGGACCTTCAGTTCCTACATCGCCTGTTGGACAGCTTCTGCCAGAGGTGATAATAGATAAGCCAGCTACTGATGACA
ATGGAACAACCACTGAAACTGAAATGAGAAAGAGAAGGTCAAGTTCTTTCCACGTTTCCATGGACTTTCTAGAAGATC
CTTCCCAAAGGCAACGAGCAATGAGTATAGCCAGCATTCTAACAAATACAGTAGAAGAACTTGAAGAATCCAGGCAGA
AATGCCCACCCTGTTGGTATAAATTTTCCAACATATTCTTAATCTGGGACTGTTCTCCATATTGGTTAAAAGTGAAAC
ATGTTGTCAACCTGGTTGTGATGGACCCATTTGTTGACCTGGCCATCACCATCTGTATTGTCTTAAATACTCTTTTCA
TGGCCATGGAGCACTATCCAATGACGGACCATTTCAATAATGTGCTTACAGTAGGAAACTTGGTTTTCACTGGGATCT
TTACAGCAGAAATGTTTCTGAAAATTATTGCCATGGATCCTTACTATTATTTCCAAGAAGGCTGGAATATCTTTGACG
GTTTTATTGTGACGCTTAGCCTGGTAGAACTTGGACTCGCCAATGTGGAAGGATTATCTGTTCTCCGTTCATTTCGAT
TGCTGCGAGTTTTCAAGTTGGCAAAATCTTGGCCAACGTTAAATATGCTAATAAAGATCATCGGCAATTCCGTGGGGG
CTCTGGGAAATTTAACCCTCGTCTTGGCCATCATCGTCTTCATTTTTGCCGTGGTCGGCATGCAGCTCTTTGGTAAAA
GCTACAAAGATTGTGTCTGCAAGATCGCCAGTGATTGTCAACTCCCACGCTGGCACATGAATGACTTCTTCCACTCCT
TCCTGATTGTGTTCCGCGTGCTGTGTGGGGAGTGGATAGAGACCATGTGGGACTGTATGGAGGTTGCTGGTCAAGCCA
TGTGCCTTACTGTCTTCATGATGGTCATGGTGATTGGAAACCTAGTGGTCCTGAATCTCTTTCTGGCCTTGCTTCTGA
GCTCATTTAGTGCAGACAACCTTGCAGCCACTGATGATGATAATGAAATGAATAATCTCCAAATTGCTGTGGATAGGA
TGCACAAAGGAGTAGCTTATGTGAAAAGAAAAATATATGAATTTATTCAACAGTCCTTCATTAGGAAACAAAAGATTT
TAGATGAAATTAAACCACTTGATGATCTAAACAACAAGAAAGACAGTTGTATGTCCAATCATACAGCAGAAATTGGGA
AAGATCTTGACTATCTTAAAGATGTAAATGGAACTACAAGTGGTATAGGAACTGGCAGCAGTGTTGAAAAATACATTA
TTGATGAAAGTGATTACATGTCATTCATAAACAACCCCAGTCTTACTGTGACTGTACCAATTGCTGTAGGAGAATCTG
ACTTTGAAAATTTAAACACGGAAGACTTTAGTAGTGAATCGGATCTGGAAGAAAGCAAAGAGAAACTGAATGAAAGCA
GTAGCTCATCAGAAGGTAGCACTGTGGACATCGGCGCACCTGTAGAAGAACAGCCCGTAGTGGAACCTGAAGAAACTC
TTGAACCAGAAGCTTGTTTCACTGAAGGCTGTGTACAAAGATTCAAGTGTTGTCAAATCAATGTGGAAGAAGGCAGAG
GAAAACAATGGTGGAACCTGAGAAGGACGTGTTTCCGAATAGTTGAACATAACTGGTTTGAGACCTTCATTGTTTTCA
TGATTCTCCTTAGTAGTGGTGCTCTGGCATTTGAAGATATATATATTGATCAGCGAAAGACGATTAAGACGATGTTGG
AATATGCTGACAAGGTTTTCACTTACATTTTCATTCTGGAAATGCTTCTAAAATGGGTGGCATATGGCTATCAAACAT
ATTTCACCAATGCCTGGTGTTGGCTGGACTTCTTAATTGTTGATGTTTCATTGGTCAGTTTAACAGCAAATGCCTTGG
GTTACTCAGAACTTGGAGCCATCAAATCTCTCAGGACACTAAGAGCTCTGAGACCTCTAAGAGCCTTATCTCGATTTG
AAGGGATGAGGGTGGTTGTGAATGCCCTTTTAGGAGCAATTCCATCCATCATGAATGTGCTTCTGGTTTGTCTTATAT
TCTGGCTAATTTTCAGCATCATGGGCGTAAATTTGTTTGCTGGCAAATTCTACCACTGTATTAACACCACAACTGGTG
ACAGGTTTGACATCGAAGACGTGAATAATCATACTGATTGCCTAAAACTAATAGAAAGAAATGAGACTGCTCGATGGA
AAAATGTGAAAGTAAACTTTGATAATGTAGGATTTGGGTATCTCTCTTTGCTTCAAGTTGCCACATTCAAAGGATGGA
TGGATATAATGTATGCAGCAGTTGATTCCAGAAATGTGGAACTCCAGCCTAAGTATGAAGAAAGTCTGTACATGTATC
TTTACTTTGTTATTTTCATCATCTTTGGGTCCTTCTTCACCTTGAACCTGTTTATTGGTGTCATCATAGATAATTTCA
ACCAGCAGAAAAAGAAGTTTGGAGGTCAAGACATCTTTATGACAGAAGAACAGAAGAAATACTATAATGCAATGAAAA
AATTAGGATCGAAAAAACCGCAAAAGCCTATACCTCGACCAGGAAACAAATTTCAAGGAATGGTCTTTGACTTCGTAA
CCAGACAAGTTTTTGACATAAGCATCATGATTCTCATCTGTCTTAACATGGTCACAATGATGGTGGAAACAGATGACC
AGAGTGAATATGTGACTACCATTTTGTCACGCATCAATCTGGTGTTCATTGTGCTATTTACTGGAGAGTGTGTACTGA
AACTCATCTCTCTACGCCATTATTATTTTACCATTGGATGGAATATTTTTGATTTTGTGGTTGTCATTCTCTCCATTG
TAGGTATGTTTCTTGCCGAGCTGATAGAAAAGTATTTCGTGTCCCCTACCCTGTTCCGAGTGATCCGTCTTGCTAGGA
TTGGCCGAATCCTACGTCTGATCAAAGGAGCAAAGGGGATCCGCACGCTGCTCTTTGCTTTGATGATGTCCCTTCCTG
CGTTGTTTAACATCGGCCTCCTACTCTTCCTAGTCATGTTCATCTACGCCATCTTTGGGATGTCCAACTTTGCCTATG
TTAAGAGGGAAGTTGGGATCGATGACATGTTCAACTTTGAGACCTTTGGCAACAGCATGATCTGCCTATTCCAAATTA
CAACCTCTGCTGGCTGGGATGGATTGCTAGCACCCATTCTCAACAGTAAGCCACCCGACTGTGACCCTAATAAAGTTA
ACCCTGGAAGCTCAGTTAAGGGAGACTGTGGGAACCCATCTGTTGGAATTTTCTTTTTTGTCAGTTACATCATCATAT
CCTTCCTGGTTGTGGTGAACATGTACATCGCGGTCATCCTGGAGAACTTCAGTGTTGCTACTGAAGAAAGTGCAGAGC
CTCTGAGTGAGGATGACTTTGAGATGTTCTATGAGGTTTGGGAGAAGTTTGATCCCGATGCAACTCAGTTCATGGAAT
TTGAAAAATTATCTCAGTTTGCAGCTGCGCTTGAACCGCCTCTCAATCTGCCACAACCAAACAAACTCCAGCTCATTG
CCATGGATTTGCCCATGGTGAGTGGTGACCGGATCCACTGTCTTGATATCTTATTTGCTTTTACAAAGCGGGTTCTAG
GAGAGAGTGGAGAGATGGATGCTCTACGAATACAGATGGAAGAGCGATTCATGGCTTCCAATCCTTCCAAGGTCTCCT
ATCAGCCAATCACTACTACTTTAAAACGAAAACAAGAGGAAGTATCTGCTGTCATTATTCAGCGTGCTTACAGACGCC
ACCTTTTAAAGCGAACTGTAAAACAAGCTTCCTTTACGTACAATAAAAACAAAATCAAAGGTGGGGCTAATCTTCTTA
TAAAAGAAGACATGATAATTGACAGAATAAATGAAAACTCTATTACAGAAAAAACTGATCTGACCATGTCCACTGCAG
CTTGTCCACCTTCCTATGACCGGGTGACAAAGCCAATTGTGGAAAAACATGAGCAAGAAGGCAAAGATGAAAAAGCCA
AAGGGAAATAAATGAAAATAAATAAAAATAATTGGGTGACAAATTGTTTACAGCCTGTGAAGGTGATGTATTTTTATC
AACAGGACTCCTTTAGGAGGTCAATGCCAAACTGACTGTTTTTACACAAATCTCCTTAAGGTCAGTGCCTACAATAAG
ACAGTGACCCCTTGTCAGCAAACTGTGACTCTGTGTAAAGGGGAGATGACCTTGACAGGAGGTTACTGTTCTCACTAC
CAGCTGACACTGCTGAAGATAAGATGCACAATGGCTAGTCAGACTGTAGGGACCAGTTTCAAGGGGTGCAAACCTGTG
ATTTTGGGGTTGTTTAACATGAAACACTTTAGTGTAGTAATTGTATCCACTGTTTGCATTTCAACTGCCACATTTGTC
ACATTTTTATGGAATCTGTTAGTGGATTCATCTTTTTGTTAATCCATGTGTTTATTATATGTGACTATTTTTGTAAAC
GAAGTTTCTGTTGAGAAATAGGCTAAGGACCTCTATAACAGGTATGCCACCTGGGGGGTATGGCAACCACATGGCCCT
CCCAGCTACACAAAGTCGTGGTTTGCATGAGGGCATGCTGCACTTAGAGATCATGCATGAGAAAAAGTCACAAGAAAA
ACAAATTCTTAAATTTCACCATATTTCTGGGAGGGGTAATTGGGTGATAAGTGGAGGTGCTTTGTTGATCTTGTTTTG
CGAAATCCAGCCCCTAGACCAAGTAGATTATTTGTGGGTAGGCCAGTAAATCTTAGCAGGTGCAAACTTCATTCAAAT
GTTTGGAGTCATAAATGTTATGTTTCTTTTTGTTGTATTAAAAAAAAAACCTGAATAGTGAATATTGCCCCTCACCCT
CCACCGCCAGAAGACTGAATTGACCAAAATTACTCTTTATAAATTTCTGCTTTTTCCTGCACTTTGTTTAGCCATCTT
CGGCTCTCAGCAAGGTTGACACTGTATATGTTAATGAAATGCTATTTATTATGTAAATAGTCATTTTACCCTGTGGTG
CACGTTTGAGCAAACAAATAATGACCTAAGCACAGTATTTATTGCATCAAATATGTACCACAAGAAATGTAGAGTGCA
AGCTTTACACAGGTAATAAAATGTATTCTGTACCATTTATAGATAGTTTGGATGCTATCAATGCATGTTTATATTACC
ATGCTGCTGTATCTGGTTTCTCTCACTGCTCAGAATCTCATTTATGAGAAACCATATGTCAGTGGTAAAGTCAAGGAA
ATTGTTCAACAGATCTCATTTATTTAAGTCATTAAGCAATAGTTTGCAGCACTTTAACAGCTTTTTGGTTATTTTTAC
ATTTTAAGTGGATAACATATGGTATATAGCCAGACTGTACAGACATGTTTAAAAAAACACACTGCTTAACCTATTAAA
TATGTGTTTAGAATTTTATAAGCAAATATAAATACTGTAAAAAGTCACTTTATTTTATTTTTCAGCATTATGTACATA
AATATGAAGAGGAAATTATCTTCAGGTTGATATCACAATCACTTTTCTTACTTTCTGTCCATAGTACTTTTTCATGAA
AGAAATTTGCTAAATAAGACATGAAAACAAGACTGGGTAGTTGTAGATTTCTGCTTTTTAAATTACATTTGCTAATTT
TAGATTATTTCACAATTTTAAGGAGCAAAATAGGTTCACGATTCATATCCAAATTATGCTTTGCAATTGGAAAAGGGT
TTAAAATTTTATTTATATTTCTGGTAGTACCTGCACTAACTGAATTGAAGGTAGTGCTTATGTTATTTTTGTTCTTTT
TTTCTGACTTCGGTTTATGTTTTCATTTCTTTGGAGTAATGCTGCTCTAGATTGTTCTAAATAGAATGTGGGCTTCAT
AATTTTTTTTTCCACAAAAACAGAGTAGTCAACTTATATAGTCAATTACATCAGGACATTTTGTGTTTCTTACAGAAG
CAAACCATAGGCTCCTCTTTTCCTTAAAACTACTTAGATAAACTGTATTCGTGAACTGCATGCTGGAAAATGCTACTA
TTATGCTAAATAATGCTAACCAACATTTAAAATGTGCAAAACTAATAAAGATTACATTTTTTATTTTATTGTTTGCCC
AGTCACTTTTTGTTAACAGAATATTCTAATGATATGGAGATTTTTTACATTACAAATTGGGGGAGAAGGGGAGCGCGC
GCGCACACACACACACACACACACACACACACACACACACACAGAGGCATACCCACGTTGACAACAAAACCTAGGGTA
GATATGTCACTGGAGGTAGGGGGTAATGACCTCCCAGAATTACAAGCAGCAGGTGTGTTCTCTGTTAGGAGGAAGAAC
TGGTGTCAGAGGATAGCTAGTGATTCTAGGAGGAAGAGAAGTATGGAAGCCAGAGTGATGGTGGATGACCCCTTGAGC
TATGAAAAGAAACCCTTAAATCATCATTTAAAAATTTAGAATTGCCATGTGTGTAGGATACTGTGTTTGCTCCTCCAG
AGCCACTCTCTCTGCTTCTGCATCATTCTGTGTGTCCCAGAAGGGTGACTTCTACACATTGCAAAAATGGGCTCTCCT
ACCTTTGAGCTCCCAATTGGTTTGGCCAATGAGAAGCACCAGTGGGAAAGCACCAGAGAGAGAAGATTGACATAGGAA
TATTTCTTCTCCAATTCCTTCTTTGCTGGGTTGGCACTGGACTCATTCCTCCCCGAAAAGTCATACTCCAATCAGACT
GCCCCTCATACAACTGAAGCTACTTTCTCTGGGGTCAGGTAATCACTCCTCCCCTTGCTCCTTCAGGTCTGCTGCTGC
ATTGAGAGTGCTTTTGTATTCCTTGTAGCTTTCTCCTAACATTGCTGACACTTTTGTAAATGTCCCCTTCATGAAATT
CTTCTATATGCCTCATTTCAGCATGCCATCTGTCTCCTGCCTGGCTGACACAAGGTGATTCAACAGCTCATGAAAGTC
AGCAGGAAGCAAAGATGTGCCTTGCTTCAGCTTGGGGTCTTAATCTTGCTAACTTTTGCAGATAAAGAAAAACAGTAA
CTGGGGGAACCACAGTGAAGTCCAGTGCAGAATTCACAGATATCATGGAAAGGTTACTCGGGTGGTCCAGATAGTAAA
ATTAACAGTCTAAATTAATCTATCTAAATTTCTGAGGAACGAGAAGCCTTCCCTTGTCATCAGGTGAAGCCAGAAGAG
GGAATATAGCCTCAACCAGAAAAGGGACAGTAATTAAAAGGCTTTTCCCATCCTTGTACAATGGACTGACTTTGCCTC
TTCATAACATCACAATCCTAAAGCAACACAACAATTAATTCTGATATATTAGTAGCTGAAAAAAATTCCCATTTCCAA
CTAAGGTAGGTCAGAATTATAGGATAAACCCTGCAGACTTTTTATACTACCCATCCACGCCATTACTCACTGTTACCT
TTCCAAATACAAAGAGAAGAACTGGTAAAACATAATCATATAAATCTCCATATTCATTTTGAAATATTTGGCATGATA
TTTTCTGTGCTAAAAAGTAATTATTCTTCAAAGAATGATGAGGTCATGTCAGTAAGACACAGGAACCAACTAGAAGGG
GCTTCCCACTGGCCAAATCTGGGGCAAGTTGAGCATCAAAATAAATGATAGTAAAAGATTATAATTCATTGAATAAGA
ATCAGCAAATACATACTGATGTAAGTAAATAAGGAAAAGTACAAATCTGTTTCTTGCAGTTGAATGTTAATTAACAAT
TGTAGAAGAAATAACGGAGTTAGAAAAATCACTATTTGGCAATCACCCTAATGACAATTGATTCATACAAGAATCATC
AATGAGTATTAAAACTCATGGGTGAAAGTTTGATGAGGAATAGGGTATTTATAGCATCTTAAAGTATCTCTTCTCTAT
TAAGTAGAAAATTTAAACAGAAGAAAGTATACTTTGGAGAAATACAGCAGACAATACCTTCAAAGATATCATCAATTA
TGAGACCAACTGATACTATGTGCCTCCTGATAAGATATACTGAAAGGGCCACATTACTTCTTGGTACACAGTCAAAAT
TTTAAAACCAGAATCTAACTACAAGGAAAATCAAATTGAGGACACTCTATAAAATAAGTGGACTGAACTCCTTAAAAA
TGTCAATGTCATGAAAGACAAAGAAAGGCTAAAGAATTCCATGAGGTCAAAGAACTATGACAACTAAACACAATTCTG
GATGGAATATCAAATTAAAAAATAACAGATAAATAATATTATTGGGAAAGTTGAATAAATTTGAATATGGACTGTTTA
TTAGTTATTAGTATTATAATAGTGTTAATTTTCCTAATTTTGTTAAGACTAGTGTGCCTGTTCCATGAAAATAGAAAA
TGTTCTTATTCTCTGAAAATGCATGCTAAAGTATTTAGGGGTGAATGCAACAATGTCTGCAGCTCATTCTTGAATCAG
TTCAAAGAAAAATGAGTTACATTTATATATATATGTATGTATGTAAACAGACATAGATAAAAGTATAGATGTGTGTGT
GTCTTTAGAAAGGGGAGGATTTTTTTTTTTTTTTTGCTGTGTGTTACTGAAGTGCCTATGTCTGCGTGTTCACACTAT
CATATTTTGTATGCCCTGGACTTTATAATTTCTACCTTCAAAATTAGATCTACTGTTGGTAATTAATTCAATATATAC
TGGTTTTTTAACTACTATTCTCATTTCCTAGCAGTAATCTTCCTGAAAAGTCACAGAAATGATTACATTCCTTGTTCT
TCATAATAATCACTGTTTAATTAAAATAAGAATATTTTAGAAAAGATCTGCGGCATAGTGGTTAAGACCCCAGTATTT
GATGCTAAACAGATCTGATTTGGATAACAGAAGGTGGCACTTTGCTGTTTAAGCTGGGGACCAGACACTGTGGGTATA
AATAGTAATTCCAAACACAGCTCCACAGAGCAGCACCCTTATGACAAGGTTTTCATATGTCTATAGTTAAGCCAGAAA
ATTAAGAATAATGCCATAAATATTTATAAAGCTGAACATATCCAAGTTAAAGACCTTTATCCTGAAATTGTATCTTTT
AGATTATTTTCTAAAGACTAATACCATTTAATGTTTAAATGTTCTTTGGAAATGATGGTGAGAATACGTGATAATGGG
TCATTGGTTTTAATATTTTATTTAGCCAAGTGGAAAATTGGCAACCTGGTGTCGGTCCTCCCATTTGTATTTTACTGG
TGCATGAAATCCAAAAGTCTAGTAACCATTGGGACAGACAACTCTACTGCATAAGTTTGTATGTTTGTATATCTGTAT
CACAAAGCCCAGACACTCGAACTATATAAACTTGTCGCACTAAAGACAGCAAATATGTCTGGTAATTGCATATTCTTC
ATGTGTGCACTGGAATTTCTTATTATATAAGAAAATAAATGTGTTTCTAAACCACCATGAATTGGGTACTGCTGATAG
TACTATTCTTCCTGGCACATGGAAATATCCCACTGAGGTTGTCAATCAATATTGATTTAATCAGTGTGGCAGCAAAGG
CACTAGAGGAGGAAAATACTCTAGACAATCAAGTATAGATGGGAAAATTGCATCACCAAGAAATTCTTAAAATCACAA
GTGGATAATTTGCAATATCAATTGATTTTACTCAACTGCTTGAGTCCTTTATCATTCCGTTAGAACACTCCTCATATT
TGCAGATAACTCAGCCCCTCACCTCAGCAACAAGCAAGAGGACATCATTATAAACTTCCTCAATTTCTTTCCTATCCA
GTGAAAAATATCTAAGTCCCTTTTCCCTTCCCTTTTCCTTCCCTTCCCTTCCTTTTTCTTTCCTTCCATTCTCTTCCT
TTTTCCTTCCTTTTTCTTTCCTTCCATTCTCTTCCTTTTTCCTTCCTTTTTCTTACTTTACCTCCCCCTCCTACCTTT
CCCTTTCTCCCTTCCTCCCTCTCTCCCTTCCTTCCTCTCTCCCTCTCCTTCCTTCCTTCCTTCAATCCATACTTTTAT
ATCTTTTCAGAGATCAATTTTTCCCATTCATTTCTACATTCTCAGATACCTTGCTCCATGACTTGTTCTACTTCTCTT
CTATCTTTATTCTTTCCCTCTTGACTGATCCTTTTTGTCTGCCTTTGCATTTTCAAAATCAGTTTTTCTAATCTTAAA
AAATAACTTTGCTCAACCCTGATGCATCTTTATTCACTGTGTCATTATCTCATTATTTTTAAAGGTTAGTTTCTAAAG
CCAGGGCTCTATACTCACTACCTTCACTCTTGGTTAAAATTAAAAGAAATATAGGTCCCAGTGATATTTATGGGAAAA
TTTGACTGTGAATAATCTTGTTGATATAAACACTTTGGGTTTTCAAAATGTTATGCTAGCTGTTAACTTTCCCAGTAA
CATTATGAGGGAAGGGCTGGAAAATACAAAAGGTAGACATAGGAACCGAGAACACCACCCCTATGGAGCCAAGCTATA
CATACTCTGTCTCTGGTTATCATAAATACATCTCCTATATTCAATGCTTGCTTAGAAAGGCATGATGTCCACGGAGCA
ATTCAATTGTTCACTGTGATGGGTTTTTTTGTTTTCTCCTCATGGAATGCAACTTTTATTTAT
SEQ ID NO: 12 >NM_001353960.2 SCN1A [organism = Homo sapiens] [GeneID = 6323]
[transcript = 14]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGGCAGCAACGGCAACTGCCTCAGAACA
TTCCAGAGAGCCCAGTGCAGCAGGCAGGCTCTCAGACAGCTCATCTGAAGCCTCTAAGTTGAGTTCCAAGAGTGCTAA
GGAAAGAAGAAATCGGAGGAAGAAAAGAAAACAGAAAGAGCAGTCTGGTGGGGAAGAGAAAGATGAGGATGAATTCCA
AAAATCTGAATCTGAGGACAGCATCAGGAGGAAAGGTTTTCGCTTCTCCATTGAAGGGAACCGATTGACATATGAAAA
GAGGTACTCCTCCCCACACCAGTCTTTGTTGAGCATCCGTGGCTCCCTATTTTCACCAAGGCGAAATAGCAGAACAAG
CCTTTTCAGCTTTAGAGGGCGAGCAAAGGATGTGGGATCTGAGAACGACTTCGCAGATGATGAGCACAGCACCTTTGA
GGATAACGAGAGCCGTAGAGATTCCTTGTTTGTGCCCCGACGACACGGAGAGAGACGCAACAGCAACCTGAGTCAGAC
CAGTAGGTCATCCCGGATGCTGGCAGTGTTTCCAGCGAATGGGAAGATGCACAGCACTGTGGATTGCAATGGTGTGGT
TTCCTTGGGAACAACCACTGAAACTGAAATGAGAAAGAGAAGGTCAAGTTCTTTCCACGTTTCCATGGACTTTCTAGA
AGATCCTTCCCAAAGGCAACGAGCAATGAGTATAGCCAGCATTCTAACAAATACAGTAGAAGAACTTGAAGAATCCAG
GCAGAAATGCCCACCCTGTTGGTATAAATTTTCCAACATATTCTTAATCTGGGACTGTTCTCCATATTGGTTAAAAGT
GAAACATGTTGTCAACCTGGTTGTGATGGACCCATTTGTTGACCTGGCCATCACCATCTGTATTGTCTTAAATACTCT
TTTCATGGCCATGGAGCACTATCCAATGACGGACCATTTCAATAATGTGCTTACAGTAGGAAACTTGGTTTTCACTGG
GATCTTTACAGCAGAAATGTTTCTGAAAATTATTGCCATGGATCCTTACTATTATTTCCAAGAAGGCTGGAATATCTT
TGACGGTTTTATTGTGACGCTTAGCCTGGTAGAACTTGGACTCGCCAATGTGGAAGGATTATCTGTTCTCCGTTCATT
TCGATTGCTGCGAGTTTTCAAGTTGGCAAAATCTTGGCCAACGTTAAATATGCTAATAAAGATCATCGGCAATTCCGT
GGGGGCTCTGGGAAATTTAACCCTCGTCTTGGCCATCATCGTCTTCATTTTTGCCGTGGTCGGCATGCAGCTCTTTGG
TAAAAGCTACAAAGATTGTGTCTGCAAGATCGCCAGTGATTGTCAACTCCCACGCTGGCACATGAATGACTTCTTCCA
CTCCTTCCTGATTGTGTTCCGCGTGCTGTGTGGGGAGTGGATAGAGACCATGTGGGACTGTATGGAGGTTGCTGGTCA
AGCCATGTGCCTTACTGTCTTCATGATGGTCATGGTGATTGGAAACCTAGTGGTCCTGAATCTCTTTCTGGCCTTGCT
TCTGAGCTCATTTAGTGCAGACAACCTTGCAGCCACTGATGATGATAATGAAATGAATAATCTCCAAATTGCTGTGGA
TAGGATGCACAAAGGAGTAGCTTATGTGAAAAGAAAAATATATGAATTTATTCAACAGTCCTTCATTAGGAAACAAAA
GATTTTAGATGAAATTAAACCACTTGATGATCTAAACAACAAGAAAGACAGTTGTATGTCCAATCATACAGCAGAAAT
TGGGAAAGATCTTGACTATCTTAAAGATGTAAATGGAACTACAAGTGGTATAGGAACTGGCAGCAGTGTTGAAAAATA
CATTATTGATGAAAGTGATTACATGTCATTCATAAACAACCCCAGTCTTACTGTGACTGTACCAATTGCTGTAGGAGA
ATCTGACTTTGAAAATTTAAACACGGAAGACTTTAGTAGTGAATCGGATCTGGAAGAAAGCAAAGAGAAACTGAATGA
AAGCAGTAGCTCATCAGAAGGTAGCACTGTGGACATCGGCGCACCTGTAGAAGAACAGCCCGTAGTGGAACCTGAAGA
AACTCTTGAACCAGAAGCTTGTTTCACTGAAGGCTGTGTACAAAGATTCAAGTGTTGTCAAATCAATGTGGAAGAAGG
CAGAGGAAAACAATGGTGGAACCTGAGAAGGACGTGTTTCCGAATAGTTGAACATAACTGGTTTGAGACCTTCATTGT
TTTCATGATTCTCCTTAGTAGTGGTGCTCTGGCATTTGAAGATATATATATTGATCAGCGAAAGACGATTAAGACGAT
GTTGGAATATGCTGACAAGGTTTTCACTTACATTTTCATTCTGGAAATGCTTCTAAAATGGGTGGCATATGGCTATCA
AACATATTTCACCAATGCCTGGTGTTGGCTGGACTTCTTAATTGTTGATGTTTCATTGGTCAGTTTAACAGCAAATGC
CTTGGGTTACTCAGAACTTGGAGCCATCAAATCTCTCAGGACACTAAGAGCTCTGAGACCTCTAAGAGCCTTATCTCG
ATTTGAAGGGATGAGGGTGGTTGTGAATGCCCTTTTAGGAGCAATTCCATCCATCATGAATGTGCTTCTGGTTTGTCT
TATATTCTGGCTAATTTTCAGCATCATGGGCGTAAATTTGTTTGCTGGCAAATTCTACCACTGTATTAACACCACAAC
TGGTGACAGGTTTGACATCGAAGACGTGAATAATCATACTGATTGCCTAAAACTAATAGAAAGAAATGAGACTGCTCG
ATGGAAAAATGTGAAAGTAAACTTTGATAATGTAGGATTTGGGTATCTCTCTTTGCTTCAAGTTGCCACATTCAAAGG
ATGGATGGATATAATGTATGCAGCAGTTGATTCCAGAAATGTGGAACTCCAGCCTAAGTATGAAGAAAGTCTGTACAT
GTATCTTTACTTTGTTATTTTCATCATCTTTGGGTCCTTCTTCACCTTGAACCTGTTTATTGGTGTCATCATAGATAA
TTTCAACCAGCAGAAAAAGAAGTTTGGAGGTCAAGACATCTTTATGACAGAAGAACAGAAGAAATACTATAATGCAAT
GAAAAAATTAGGATCGAAAAAACCGCAAAAGCCTATACCTCGACCAGGAAACAAATTTCAAGGAATGGTCTTTGACTT
CGTAACCAGACAAGTTTTTGACATAAGCATCATGATTCTCATCTGTCTTAACATGGTCACAATGATGGTGGAAACAGA
TGACCAGAGTGAATATGTGACTACCATTTTGTCACGCATCAATCTGGTGTTCATTGTGCTATTTACTGGAGAGTGTGT
ACTGAAACTCATCTCTCTACGCCATTATTATTTTACCATTGGATGGAATATTTTTGATTTTGTGGTTGTCATTCTCTC
CATTGTAGGTATGTTTCTTGCCGAGCTGATAGAAAAGTATTTCGTGTCCCCTACCCTGTTCCGAGTGATCCGTCTTGC
TAGGATTGGCCGAATCCTACGTCTGATCAAAGGAGCAAAGGGGATCCGCACGCTGCTCTTTGCTTTGATGATGTCCCT
TCCTGCGTTGTTTAACATCGGCCTCCTACTCTTCCTAGTCATGTTCATCTACGCCATCTTTGGGATGTCCAACTTTGC
CTATGTTAAGAGGGAAGTTGGGATCGATGACATGTTCAACTTTGAGACCTTTGGCAACAGCATGATCTGCCTATTCCA
AATTACAACCTCTGCTGGCTGGGATGGATTGCTAGCACCCATTCTCAACAGTAAGCCACCCGACTGTGACCCTAATAA
AGTTAACCCTGGAAGCTCAGTTAAGGGAGACTGTGGGAACCCATCTGTTGGAATTTTCTTTTTTGTCAGTTACATCAT
CATATCCTTCCTGGTTGTGGTGAACATGTACATCGCGGTCATCCTGGAGAACTTCAGTGTTGCTACTGAAGAAAGTGC
AGAGCCTCTGAGTGAGGATGACTTTGAGATGTTCTATGAGGTTTGGGAGAAGTTTGATCCCGATGCAACTCAGTTCAT
GGAATTTGAAAAATTATCTCAGTTTGCAGCTGCGCTTGAACCGCCTCTCAATCTGCCACAACCAAACAAACTCCAGCT
CATTGCCATGGATTTGCCCATGGTGAGTGGTGACCGGATCCACTGTCTTGATATCTTATTTGCTTTTACAAAGCGGGT
TCTAGGAGAGAGTGGAGAGATGGATGCTCTACGAATACAGATGGAAGAGCGATTCATGGCTTCCAATCCTTCCAAGGT
CTCCTATCAGCCAATCACTACTACTTTAAAACGAAAACAAGAGGAAGTATCTGCTGTCATTATTCAGCGTGCTTACAG
ACGCCACCTTTTAAAGCGAACTGTAAAACAAGCTTCCTTTACGTACAATAAAAACAAAATCAAAGGTGGGGCTAATCT
TCTTATAAAAGAAGACATGATAATTGACAGAATAAATGAAAACTCTATTACAGAAAAAACTGATCTGACCATGTCCAC
TGCAGCTTGTCCACCTTCCTATGACCGGGTGACAAAGCCAATTGTGGAAAAACATGAGCAAGAAGGCAAAGATGAAAA
AGCCAAAGGGAAATAAATGAAAATAAATAAAAATAATTGGGTGACAAATTGTTTACAGCCTGTGAAGGTGATGTATTT
TTATCAACAGGACTCCTTTAGGAGGTCAATGCCAAACTGACTGTTTTTACACAAATCTCCTTAAGGTCAGTGCCTACA
ATAAGACAGTGACCCCTTGTCAGCAAACTGTGACTCTGTGTAAAGGGGAGATGACCTTGACAGGAGGTTACTGTTCTC
ACTACCAGCTGACACTGCTGAAGATAAGATGCACAATGGCTAGTCAGACTGTAGGGACCAGTTTCAAGGGGTGCAAAC
CTGTGATTTTGGGGTTGTTTAACATGAAACACTTTAGTGTAGTAATTGTATCCACTGTTTGCATTTCAACTGCCACAT
TTGTCACATTTTTATGGAATCTGTTAGTGGATTCATCTTTTTGTTAATCCATGTGTTTATTATATGTGACTATTTTTG
TAAACGAAGTTTCTGTTGAGAAATAGGCTAAGGACCTCTATAACAGGTATGCCACCTGGGGGGTATGGCAACCACATG
GCCCTCCCAGCTACACAAAGTCGTGGTTTGCATGAGGGCATGCTGCACTTAGAGATCATGCATGAGAAAAAGTCACAA
GAAAAACAAATTCTTAAATTTCACCATATTTCTGGGAGGGGTAATTGGGTGATAAGTGGAGGTGCTTTGTTGATCTTG
TTTTGCGAAATCCAGCCCCTAGACCAAGTAGATTATTTGTGGGTAGGCCAGTAAATCTTAGCAGGTGCAAACTTCATT
CAAATGTTTGGAGTCATAAATGTTATGTTTCTTTTTGTTGTATTAAAAAAAAAACCTGAATAGTGAATATTGCCCCTC
ACCCTCCACCGCCAGAAGACTGAATTGACCAAAATTACTCTTTATAAATTTCTGCTTTTTCCTGCACTTTGTTTAGCC
ATCTTCGGCTCTCAGCAAGGTTGACACTGTATATGTTAATGAAATGCTATTTATTATGTAAATAGTCATTTTACCCTG
TGGTGCACGTTTGAGCAAACAAATAATGACCTAAGCACAGTATTTATTGCATCAAATATGTACCACAAGAAATGTAGA
GTGCAAGCTTTACACAGGTAATAAAATGTATTCTGTACCATTTATAGATAGTTTGGATGCTATCAATGCATGTTTATA
TTACCATGCTGCTGTATCTGGTTTCTCTCACTGCTCAGAATCTCATTTATGAGAAACCATATGTCAGTGGTAAAGTCA
AGGAAATTGTTCAACAGATCTCATTTATTTAAGTCATTAAGCAATAGTTTGCAGCACTTTAACAGCTTTTTGGTTATT
TTTACATTTTAAGTGGATAACATATGGTATATAGCCAGACTGTACAGACATGTTTAAAAAAACACACTGCTTAACCTA
TTAAATATGTGTTTAGAATTTTATAAGCAAATATAAATACTGTAAAAAGTCACTTTATTTTATTTTTCAGCATTATGT
ACATAAATATGAAGAGGAAATTATCTTCAGGTTGATATCACAATCACTTTTCTTACTTTCTGTCCATAGTACTTTTTC
ATGAAAGAAATTTGCTAAATAAGACATGAAAACAAGACTGGGTAGTTGTAGATTTCTGCTTTTTAAATTACATTTGCT
AATTTTAGATTATTTCACAATTTTAAGGAGCAAAATAGGTTCACGATTCATATCCAAATTATGCTTTGCAATTGGAAA
AGGGTTTAAAATTTTATTTATATTTCTGGTAGTACCTGCACTAACTGAATTGAAGGTAGTGCTTATGTTATTTTTGTT
CTTTTTTTCTGACTTCGGTTTATGTTTTCATTTCTTTGGAGTAATGCTGCTCTAGATTGTTCTAAATAGAATGTGGGC
TTCATAATTTTTTTTTCCACAAAAACAGAGTAGTCAACTTATATAGTCAATTACATCAGGACATTTTGTGTTTCTTAC
AGAAGCAAACCATAGGCTCCTCTTTTCCTTAAAACTACTTAGATAAACTGTATTCGTGAACTGCATGCTGGAAAATGC
TACTATTATGCTAAATAATGCTAACCAACATTTAAAATGTGCAAAACTAATAAAGATTACATTTTTTATTTTATTGTT
TGCCCAGTCACTTTTTGTTAACAGAATATTCTAATGATATGGAGATTTTTTACATTACAAATTGGGGGAGAAGGGGAG
CGCGCGCGCACACACACACACACACACACACACACACACACACACACAGAGGCATACCCACGTTGACAACAAAACCTA
GGGTAGATATGTCACTGGAGGTAGGGGGTAATGACCTCCCAGAATTACAAGCAGCAGGTGTGTTCTCTGTTAGGAGGA
AGAACTGGTGTCAGAGGATAGCTAGTGATTCTAGGAGGAAGAGAAGTATGGAAGCCAGAGTGATGGTGGATGACCCCT
TGAGCTATGAAAAGAAACCCTTAAATCATCATTTAAAAATTTAGAATTGCCATGTGTGTAGGATACTGTGTTTGCTCC
TCCAGAGCCACTCTCTCTGCTTCTGCATCATTCTGTGTGTCCCAGAAGGGTGACTTCTACACATTGCAAAAATGGGCT
CTCCTACCTTTGAGCTCCCAATTGGTTTGGCCAATGAGAAGCACCAGTGGGAAAGCACCAGAGAGAGAAGATTGACAT
AGGAATATTTCTTCTCCAATTCCTTCTTTGCTGGGTTGGCACTGGACTCATTCCTCCCCGAAAAGTCATACTCCAATC
AGACTGCCCCTCATACAACTGAAGCTACTTTCTCTGGGGTCAGGTAATCACTCCTCCCCTTGCTCCTTCAGGTCTGCT
GCTGCATTGAGAGTGCTTTTGTATTCCTTGTAGCTTTCTCCTAACATTGCTGACACTTTTGTAAATGTCCCCTTCATG
AAATTCTTCTATATGCCTCATTTCAGCATGCCATCTGTCTCCTGCCTGGCTGACACAAGGTGATTCAACAGCTCATGA
AAGTCAGCAGGAAGCAAAGATGTGCCTTGCTTCAGCTTGGGGTCTTAATCTTGCTAACTTTTGCAGATAAAGAAAAAC
AGTAACTGGGGGAACCACAGTGAAGTCCAGTGCAGAATTCACAGATATCATGGAAAGGTTACTCGGGTGGTCCAGATA
GTAAAATTAACAGTCTAAATTAATCTATCTAAATTTCTGAGGAACGAGAAGCCTTCCCTTGTCATCAGGTGAAGCCAG
AAGAGGGAATATAGCCTCAACCAGAAAAGGGACAGTAATTAAAAGGCTTTTCCCATCCTTGTACAATGGACTGACTTT
GCCTCTTCATAACATCACAATCCTAAAGCAACACAACAATTAATTCTGATATATTAGTAGCTGAAAAAAATTCCCATT
TCCAACTAAGGTAGGTCAGAATTATAGGATAAACCCTGCAGACTTTTTATACTACCCATCCACGCCATTACTCACTGT
TACCTTTCCAAATACAAAGAGAAGAACTGGTAAAACATAATCATATAAATCTCCATATTCATTTTGAAATATTTGGCA
TGATATTTTCTGTGCTAAAAAGTAATTATTCTTCAAAGAATGATGAGGTCATGTCAGTAAGACACAGGAACCAACTAG
AAGGGGCTTCCCACTGGCCAAATCTGGGGCAAGTTGAGCATCAAAATAAATGATAGTAAAAGATTATAATTCATTGAA
TAAGAATCAGCAAATACATACTGATGTAAGTAAATAAGGAAAAGTACAAATCTGTTTCTTGCAGTTGAATGTTAATTA
ACAATTGTAGAAGAAATAACGGAGTTAGAAAAATCACTATTTGGCAATCACCCTAATGACAATTGATTCATACAAGAA
TCATCAATGAGTATTAAAACTCATGGGTGAAAGTTTGATGAGGAATAGGGTATTTATAGCATCTTAAAGTATCTCTTC
TCTATTAAGTAGAAAATTTAAACAGAAGAAAGTATACTTTGGAGAAATACAGCAGACAATACCTTCAAAGATATCATC
AATTATGAGACCAACTGATACTATGTGCCTCCTGATAAGATATACTGAAAGGGCCACATTACTTCTTGGTACACAGTC
AAAATTTTAAAACCAGAATCTAACTACAAGGAAAATCAAATTGAGGACACTCTATAAAATAAGTGGACTGAACTCCTT
AAAAATGTCAATGTCATGAAAGACAAAGAAAGGCTAAAGAATTCCATGAGGTCAAAGAACTATGACAACTAAACACAA
TTCTGGATGGAATATCAAATTAAAAAATAACAGATAAATAATATTATTGGGAAAGTTGAATAAATTTGAATATGGACT
GTTTATTAGTTATTAGTATTATAATAGTGTTAATTTTCCTAATTTTGTTAAGACTAGTGTGCCTGTTCCATGAAAATA
GAAAATGTTCTTATTCTCTGAAAATGCATGCTAAAGTATTTAGGGGTGAATGCAACAATGTCTGCAGCTCATTCTTGA
ATCAGTTCAAAGAAAAATGAGTTACATTTATATATATATGTATGTATGTAAACAGACATAGATAAAAGTATAGATGTG
TGTGTGTCTTTAGAAAGGGGAGGATTTTTTTTTTTTTTTTGCTGTGTGTTACTGAAGTGCCTATGTCTGCGTGTTCAC
ACTATCATATTTTGTATGCCCTGGACTTTATAATTTCTACCTTCAAAATTAGATCTACTGTTGGTAATTAATTCAATA
TATACTGGTTTTTTAACTACTATTCTCATTTCCTAGCAGTAATCTTCCTGAAAAGTCACAGAAATGATTACATTCCTT
GTTCTTCATAATAATCACTGTTTAATTAAAATAAGAATATTTTAGAAAAGATCTGCGGCATAGTGGTTAAGACCCCAG
TATTTGATGCTAAACAGATCTGATTTGGATAACAGAAGGTGGCACTTTGCTGTTTAAGCTGGGGACCAGACACTGTGG
GTATAAATAGTAATTCCAAACACAGCTCCACAGAGCAGCACCCTTATGACAAGGTTTTCATATGTCTATAGTTAAGCC
AGAAAATTAAGAATAATGCCATAAATATTTATAAAGCTGAACATATCCAAGTTAAAGACCTTTATCCTGAAATTGTAT
CTTTTAGATTATTTTCTAAAGACTAATACCATTTAATGTTTAAATGTTCTTTGGAAATGATGGTGAGAATACGTGATA
ATGGGTCATTGGTTTTAATATTTTATTTAGCCAAGTGGAAAATTGGCAACCTGGTGTCGGTCCTCCCATTTGTATTTT
ACTGGTGCATGAAATCCAAAAGTCTAGTAACCATTGGGACAGACAACTCTACTGCATAAGTTTGTATGTTTGTATATC
TGTATCACAAAGCCCAGACACTCGAACTATATAAACTTGTCGCACTAAAGACAGCAAATATGTCTGGTAATTGCATAT
TCTTCATGTGTGCACTGGAATTTCTTATTATATAAGAAAATAAATGTGTTTCTAAACCACCATGAATTGGGTACTGCT
GATAGTACTATTCTTCCTGGCACATGGAAATATCCCACTGAGGTTGTCAATCAATATTGATTTAATCAGTGTGGCAGC
AAAGGCACTAGAGGAGGAAAATACTCTAGACAATCAAGTATAGATGGGAAAATTGCATCACCAAGAAATTCTTAAAAT
CACAAGTGGATAATTTGCAATATCAATTGATTTTACTCAACTGCTTGAGTCCTTTATCATTCCGTTAGAACACTCCTC
ATATTTGCAGATAACTCAGCCCCTCACCTCAGCAACAAGCAAGAGGACATCATTATAAACTTCCTCAATTTCTTTCCT
ATCCAGTGAAAAATATCTAAGTCCCTTTTCCCTTCCCTTTTCCTTCCCTTCCCTTCCTTTTTCTTTCCTTCCATTCTC
TTCCTTTTTCCTTCCTTTTTCTTTCCTTCCATTCTCTTCCTTTTTCCTTCCTTTTTCTTACTTTACCTCCCCCTCCTA
CCTTTCCCTTTCTCCCTTCCTCCCTCTCTCCCTTCCTTCCTCTCTCCCTCTCCTTCCTTCCTTCCTTCAATCCATACT
TTTATATCTTTTCAGAGATCAATTTTTCCCATTCATTTCTACATTCTCAGATACCTTGCTCCATGACTTGTTCTACTT
CTCTTCTATCTTTATTCTTTCCCTCTTGACTGATCCTTTTTGTCTGCCTTTGCATTTTCAAAATCAGTTTTTCTAATC
TTAAAAAATAACTTTGCTCAACCCTGATGCATCTTTATTCACTGTGTCATTATCTCATTATTTTTAAAGGTTAGTTTC
TAAAGCCAGGGCTCTATACTCACTACCTTCACTCTTGGTTAAAATTAAAAGAAATATAGGTCCCAGTGATATTTATGG
GAAAATTTGACTGTGAATAATCTTGTTGATATAAACACTTTGGGTTTTCAAAATGTTATGCTAGCTGTTAACTTTCCC
AGTAACATTATGAGGGAAGGGCTGGAAAATACAAAAGGTAGACATAGGAACCGAGAACACCACCCCTATGGAGCCAAG
CTATACATACTCTGTCTCTGGTTATCATAAATACATCTCCTATATTCAATGCTTGCTTAGAAAGGCATGATGTCCACG
GAGCAATTCAATTGTTCACTGTGATGGGTTTTTTTGTTTTCTCCTCATGGAATGCAACTTTTATTTAT
SEQ ID NO: 13 >NM_001353954.2 SCN1A [organism = Homo sapiens] [GeneID = 6323]
[transcript = 10]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGGCAGCAACGGCAACTGCCTCAGAACA
TTCCAGAGAGCCCAGTGCAGCAGGCAGGCTCTCAGACAGCTCATCTGAAGCCTCTAAGTTGAGTTCCAAGAGTGCTAA
GGAAAGAAGAAATCGGAGGAAGAAAAGAAAACAGAAAGAGCAGTCTGGTGGGGAAGAGAAAGATGAGGATGAATTCCA
AAAATCTGAATCTGAGGACAGCATCAGGAGGAAAGGTTTTCGCTTCTCCATTGAAGGGAACCGATTGACATATGAAAA
GAGGTACTCCTCCCCACACCAGTCTTTGTTGAGCATCCGTGGCTCCCTATTTTCACCAAGGCGAAATAGCAGAACAAG
CCTTTTCAGCTTTAGAGGGCGAGCAAAGGATGTGGGATCTGAGAACGACTTCGCAGATGATGAGCACAGCACCTTTGA
GGATAACGAGAGCCGTAGAGATTCCTTGTTTGTGCCCCGACGACACGGAGAGAGACGCAACAGCAACCTGAGTCAGAC
CAGTAGGTCATCCCGGATGCTGGCAGTGTTTCCAGCGAATGGGAAGATGCACAGCACTGTGGATTGCAATGGTGTGGT
TTCCTTGGTTGGTGGACCTTCAGTTCCTACATCGCCTGTTGGACAGCTTCTGCCAGAGGGAACAACCACTGAAACTGA
AATGAGAAAGAGAAGGTCAAGTTCTTTCCACGTTTCCATGGACTTTCTAGAAGATCCTTCCCAAAGGCAACGAGCAAT
GAGTATAGCCAGCATTCTAACAAATACAGTAGAAGAACTTGAAGAATCCAGGCAGAAATGCCCACCCTGTTGGTATAA
ATTTTCCAACATATTCTTAATCTGGGACTGTTCTCCATATTGGTTAAAAGTGAAACATGTTGTCAACCTGGTTGTGAT
GGACCCATTTGTTGACCTGGCCATCACCATCTGTATTGTCTTAAATACTCTTTTCATGGCCATGGAGCACTATCCAAT
GACGGACCATTTCAATAATGTGCTTACAGTAGGAAACTTGGTTTTCACTGGGATCTTTACAGCAGAAATGTTTCTGAA
AATTATTGCCATGGATCCTTACTATTATTTCCAAGAAGGCTGGAATATCTTTGACGGTTTTATTGTGACGCTTAGCCT
GGTAGAACTTGGACTCGCCAATGTGGAAGGATTATCTGTTCTCCGTTCATTTCGATTGCTGCGAGTTTTCAAGTTGGC
AAAATCTTGGCCAACGTTAAATATGCTAATAAAGATCATCGGCAATTCCGTGGGGGCTCTGGGAAATTTAACCCTCGT
CTTGGCCATCATCGTCTTCATTTTTGCCGTGGTCGGCATGCAGCTCTTTGGTAAAAGCTACAAAGATTGTGTCTGCAA
GATCGCCAGTGATTGTCAACTCCCACGCTGGCACATGAATGACTTCTTCCACTCCTTCCTGATTGTGTTCCGCGTGCT
GTGTGGGGAGTGGATAGAGACCATGTGGGACTGTATGGAGGTTGCTGGTCAAGCCATGTGCCTTACTGTCTTCATGAT
GGTCATGGTGATTGGAAACCTAGTGGTCCTGAATCTCTTTCTGGCCTTGCTTCTGAGCTCATTTAGTGCAGACAACCT
TGCAGCCACTGATGATGATAATGAAATGAATAATCTCCAAATTGCTGTGGATAGGATGCACAAAGGAGTAGCTTATGT
GAAAAGAAAAATATATGAATTTATTCAACAGTCCTTCATTAGGAAACAAAAGATTTTAGATGAAATTAAACCACTTGA
TGATCTAAACAACAAGAAAGACAGTTGTATGTCCAATCATACAGCAGAAATTGGGAAAGATCTTGACTATCTTAAAGA
TGTAAATGGAACTACAAGTGGTATAGGAACTGGCAGCAGTGTTGAAAAATACATTATTGATGAAAGTGATTACATGTC
ATTCATAAACAACCCCAGTCTTACTGTGACTGTACCAATTGCTGTAGGAGAATCTGACTTTGAAAATTTAAACACGGA
AGACTTTAGTAGTGAATCGGATCTGGAAGAAAGCAAAGAGAAACTGAATGAAAGCAGTAGCTCATCAGAAGGTAGCAC
TGTGGACATCGGCGCACCTGTAGAAGAACAGCCCGTAGTGGAACCTGAAGAAACTCTTGAACCAGAAGCTTGTTTCAC
TGAAGGCTGTGTACAAAGATTCAAGTGTTGTCAAATCAATGTGGAAGAAGGCAGAGGAAAACAATGGTGGAACCTGAG
AAGGACGTGTTTCCGAATAGTTGAACATAACTGGTTTGAGACCTTCATTGTTTTCATGATTCTCCTTAGTAGTGGTGC
TCTGGCATTTGAAGATATATATATTGATCAGCGAAAGACGATTAAGACGATGTTGGAATATGCTGACAAGGTTTTCAC
TTACATTTTCATTCTGGAAATGCTTCTAAAATGGGTGGCATATGGCTATCAAACATATTTCACCAATGCCTGGTGTTG
GCTGGACTTCTTAATTGTTGATGTTTCATTGGTCAGTTTAACAGCAAATGCCTTGGGTTACTCAGAACTTGGAGCCAT
CAAATCTCTCAGGACACTAAGAGCTCTGAGACCTCTAAGAGCCTTATCTCGATTTGAAGGGATGAGGGTGGTTGTGAA
TGCCCTTTTAGGAGCAATTCCATCCATCATGAATGTGCTTCTGGTTTGTCTTATATTCTGGCTAATTTTCAGCATCAT
GGGCGTAAATTTGTTTGCTGGCAAATTCTACCACTGTATTAACACCACAACTGGTGACAGGTTTGACATCGAAGACGT
GAATAATCATACTGATTGCCTAAAACTAATAGAAAGAAATGAGACTGCTCGATGGAAAAATGTGAAAGTAAACTTTGA
TAATGTAGGATTTGGGTATCTCTCTTTGCTTCAAGTTGCCACATTCAAAGGATGGATGGATATAATGTATGCAGCAGT
TGATTCCAGAAATGTGGAACTCCAGCCTAAGTATGAAGAAAGTCTGTACATGTATCTTTACTTTGTTATTTTCATCAT
CTTTGGGTCCTTCTTCACCTTGAACCTGTTTATTGGTGTCATCATAGATAATTTCAACCAGCAGAAAAAGAAGTTTGG
AGGTCAAGACATCTTTATGACAGAAGAACAGAAGAAATACTATAATGCAATGAAAAAATTAGGATCGAAAAAACCGCA
AAAGCCTATACCTCGACCAGGAAACAAATTTCAAGGAATGGTCTTTGACTTCGTAACCAGACAAGTTTTTGACATAAG
CATCATGATTCTCATCTGTCTTAACATGGTCACAATGATGGTGGAAACAGATGACCAGAGTGAATATGTGACTACCAT
TTTGTCACGCATCAATCTGGTGTTCATTGTGCTATTTACTGGAGAGTGTGTACTGAAACTCATCTCTCTACGCCATTA
TTATTTTACCATTGGATGGAATATTTTTGATTTTGTGGTTGTCATTCTCTCCATTGTAGGTATGTTTCTTGCCGAGCT
GATAGAAAAGTATTTCGTGTCCCCTACCCTGTTCCGAGTGATCCGTCTTGCTAGGATTGGCCGAATCCTACGTCTGAT
CAAAGGAGCAAAGGGGATCCGCACGCTGCTCTTTGCTTTGATGATGTCCCTTCCTGCGTTGTTTAACATCGGCCTCCT
ACTCTTCCTAGTCATGTTCATCTACGCCATCTTTGGGATGTCCAACTTTGCCTATGTTAAGAGGGAAGTTGGGATCGA
TGACATGTTCAACTTTGAGACCTTTGGCAACAGCATGATCTGCCTATTCCAAATTACAACCTCTGCTGGCTGGGATGG
ATTGCTAGCACCCATTCTCAACAGTAAGCCACCCGACTGTGACCCTAATAAAGTTAACCCTGGAAGCTCAGTTAAGGG
AGACTGTGGGAACCCATCTGTTGGAATTTTCTTTTTTGTCAGTTACATCATCATATCCTTCCTGGTTGTGGTGAACAT
GTACATCGCGGTCATCCTGGAGAACTTCAGTGTTGCTACTGAAGAAAGTGCAGAGCCTCTGAGTGAGGATGACTTTGA
GATGTTCTATGAGGTTTGGGAGAAGTTTGATCCCGATGCAACTCAGTTCATGGAATTTGAAAAATTATCTCAGTTTGC
AGCTGCGCTTGAACCGCCTCTCAATCTGCCACAACCAAACAAACTCCAGCTCATTGCCATGGATTTGCCCATGGTGAG
TGGTGACCGGATCCACTGTCTTGATATCTTATTTGCTTTTACAAAGCGGGTTCTAGGAGAGAGTGGAGAGATGGATGC
TCTACGAATACAGATGGAAGAGCGATTCATGGCTTCCAATCCTTCCAAGGTCTCCTATCAGCCAATCACTACTACTTT
AAAACGAAAACAAGAGGAAGTATCTGCTGTCATTATTCAGCGTGCTTACAGACGCCACCTTTTAAAGCGAACTGTAAA
ACAAGCTTCCTTTACGTACAATAAAAACAAAATCAAAGGTGGGGCTAATCTTCTTATAAAAGAAGACATGATAATTGA
CAGAATAAATGAAAACTCTATTACAGAAAAAACTGATCTGACCATGTCCACTGCAGCTTGTCCACCTTCCTATGACCG
GGTGACAAAGCCAATTGTGGAAAAACATGAGCAAGAAGGCAAAGATGAAAAAGCCAAAGGGAAATAAATGAAAATAAA
TAAAAATAATTGGGTGACAAATTGTTTACAGCCTGTGAAGGTGATGTATTTTTATCAACAGGACTCCTTTAGGAGGTC
AATGCCAAACTGACTGTTTTTACACAAATCTCCTTAAGGTCAGTGCCTACAATAAGACAGTGACCCCTTGTCAGCAAA
CTGTGACTCTGTGTAAAGGGGAGATGACCTTGACAGGAGGTTACTGTTCTCACTACCAGCTGACACTGCTGAAGATAA
GATGCACAATGGCTAGTCAGACTGTAGGGACCAGTTTCAAGGGGTGCAAACCTGTGATTTTGGGGTTGTTTAACATGA
AACACTTTAGTGTAGTAATTGTATCCACTGTTTGCATTTCAACTGCCACATTTGTCACATTTTTATGGAATCTGTTAG
TGGATTCATCTTTTTGTTAATCCATGTGTTTATTATATGTGACTATTTTTGTAAACGAAGTTTCTGTTGAGAAATAGG
CTAAGGACCTCTATAACAGGTATGCCACCTGGGGGGTATGGCAACCACATGGCCCTCCCAGCTACACAAAGTCGTGGT
TTGCATGAGGGCATGCTGCACTTAGAGATCATGCATGAGAAAAAGTCACAAGAAAAACAAATTCTTAAATTTCACCAT
ATTTCTGGGAGGGGTAATTGGGTGATAAGTGGAGGTGCTTTGTTGATCTTGTTTTGCGAAATCCAGCCCCTAGACCAA
GTAGATTATTTGTGGGTAGGCCAGTAAATCTTAGCAGGTGCAAACTTCATTCAAATGTTTGGAGTCATAAATGTTATG
TTTCTTTTTGTTGTATTAAAAAAAAAACCTGAATAGTGAATATTGCCCCTCACCCTCCACCGCCAGAAGACTGAATTG
ACCAAAATTACTCTTTATAAATTTCTGCTTTTTCCTGCACTTTGTTTAGCCATCTTCGGCTCTCAGCAAGGTTGACAC
TGTATATGTTAATGAAATGCTATTTATTATGTAAATAGTCATTTTACCCTGTGGTGCACGTTTGAGCAAACAAATAAT
GACCTAAGCACAGTATTTATTGCATCAAATATGTACCACAAGAAATGTAGAGTGCAAGCTTTACACAGGTAATAAAAT
GTATTCTGTACCATTTATAGATAGTTTGGATGCTATCAATGCATGTTTATATTACCATGCTGCTGTATCTGGTTTCTC
TCACTGCTCAGAATCTCATTTATGAGAAACCATATGTCAGTGGTAAAGTCAAGGAAATTGTTCAACAGATCTCATTTA
TTTAAGTCATTAAGCAATAGTTTGCAGCACTTTAACAGCTTTTTGGTTATTTTTACATTTTAAGTGGATAACATATGG
TATATAGCCAGACTGTACAGACATGTTTAAAAAAACACACTGCTTAACCTATTAAATATGTGTTTAGAATTTTATAAG
CAAATATAAATACTGTAAAAAGTCACTTTATTTTATTTTTCAGCATTATGTACATAAATATGAAGAGGAAATTATCTT
CAGGTTGATATCACAATCACTTTTCTTACTTTCTGTCCATAGTACTTTTTCATGAAAGAAATTTGCTAAATAAGACAT
GAAAACAAGACTGGGTAGTTGTAGATTTCTGCTTTTTAAATTACATTTGCTAATTTTAGATTATTTCACAATTTTAAG
GAGCAAAATAGGTTCACGATTCATATCCAAATTATGCTTTGCAATTGGAAAAGGGTTTAAAATTTTATTTATATTTCT
GGTAGTACCTGCACTAACTGAATTGAAGGTAGTGCTTATGTTATTTTTGTTCTTTTTTTCTGACTTCGGTTTATGTTT
TCATTTCTTTGGAGTAATGCTGCTCTAGATTGTTCTAAATAGAATGTGGGCTTCATAATTTTTTTTTCCACAAAAACA
GAGTAGTCAACTTATATAGTCAATTACATCAGGACATTTTGTGTTTCTTACAGAAGCAAACCATAGGCTCCTCTTTTC
CTTAAAACTACTTAGATAAACTGTATTCGTGAACTGCATGCTGGAAAATGCTACTATTATGCTAAATAATGCTAACCA
ACATTTAAAATGTGCAAAACTAATAAAGATTACATTTTTTATTTTATTGTTTGCCCAGTCACTTTTTGTTAACAGAAT
ATTCTAATGATATGGAGATTTTTTACATTACAAATTGGGGGAGAAGGGGAGCGCGCGCGCACACACACACACACACAC
ACACACACACACACACACACAGAGGCATACCCACGTTGACAACAAAACCTAGGGTAGATATGTCACTGGAGGTAGGGG
GTAATGACCTCCCAGAATTACAAGCAGCAGGTGTGTTCTCTGTTAGGAGGAAGAACTGGTGTCAGAGGATAGCTAGTG
ATTCTAGGAGGAAGAGAAGTATGGAAGCCAGAGTGATGGTGGATGACCCCTTGAGCTATGAAAAGAAACCCTTAAATC
ATCATTTAAAAATTTAGAATTGCCATGTGTGTAGGATACTGTGTTTGCTCCTCCAGAGCCACTCTCTCTGCTTCTGCA
TCATTCTGTGTGTCCCAGAAGGGTGACTTCTACACATTGCAAAAATGGGCTCTCCTACCTTTGAGCTCCCAATTGGTT
TGGCCAATGAGAAGCACCAGTGGGAAAGCACCAGAGAGAGAAGATTGACATAGGAATATTTCTTCTCCAATTCCTTCT
TTGCTGGGTTGGCACTGGACTCATTCCTCCCCGAAAAGTCATACTCCAATCAGACTGCCCCTCATACAACTGAAGCTA
CTTTCTCTGGGGTCAGGTAATCACTCCTCCCCTTGCTCCTTCAGGTCTGCTGCTGCATTGAGAGTGCTTTTGTATTCC
TTGTAGCTTTCTCCTAACATTGCTGACACTTTTGTAAATGTCCCCTTCATGAAATTCTTCTATATGCCTCATTTCAGC
ATGCCATCTGTCTCCTGCCTGGCTGACACAAGGTGATTCAACAGCTCATGAAAGTCAGCAGGAAGCAAAGATGTGCCT
TGCTTCAGCTTGGGGTCTTAATCTTGCTAACTTTTGCAGATAAAGAAAAACAGTAACTGGGGGAACCACAGTGAAGTC
CAGTGCAGAATTCACAGATATCATGGAAAGGTTACTCGGGTGGTCCAGATAGTAAAATTAACAGTCTAAATTAATCTA
TCTAAATTTCTGAGGAACGAGAAGCCTTCCCTTGTCATCAGGTGAAGCCAGAAGAGGGAATATAGCCTCAACCAGAAA
AGGGACAGTAATTAAAAGGCTTTTCCCATCCTTGTACAATGGACTGACTTTGCCTCTTCATAACATCACAATCCTAAA
GCAACACAACAATTAATTCTGATATATTAGTAGCTGAAAAAAATTCCCATTTCCAACTAAGGTAGGTCAGAATTATAG
GATAAACCCTGCAGACTTTTTATACTACCCATCCACGCCATTACTCACTGTTACCTTTCCAAATACAAAGAGAAGAAC
TGGTAAAACATAATCATATAAATCTCCATATTCATTTTGAAATATTTGGCATGATATTTTCTGTGCTAAAAAGTAATT
ATTCTTCAAAGAATGATGAGGTCATGTCAGTAAGACACAGGAACCAACTAGAAGGGGCTTCCCACTGGCCAAATCTGG
GGCAAGTTGAGCATCAAAATAAATGATAGTAAAAGATTATAATTCATTGAATAAGAATCAGCAAATACATACTGATGT
AAGTAAATAAGGAAAAGTACAAATCTGTTTCTTGCAGTTGAATGTTAATTAACAATTGTAGAAGAAATAACGGAGTTA
GAAAAATCACTATTTGGCAATCACCCTAATGACAATTGATTCATACAAGAATCATCAATGAGTATTAAAACTCATGGG
TGAAAGTTTGATGAGGAATAGGGTATTTATAGCATCTTAAAGTATCTCTTCTCTATTAAGTAGAAAATTTAAACAGAA
GAAAGTATACTTTGGAGAAATACAGCAGACAATACCTTCAAAGATATCATCAATTATGAGACCAACTGATACTATGTG
CCTCCTGATAAGATATACTGAAAGGGCCACATTACTTCTTGGTACACAGTCAAAATTTTAAAACCAGAATCTAACTAC
AAGGAAAATCAAATTGAGGACACTCTATAAAATAAGTGGACTGAACTCCTTAAAAATGTCAATGTCATGAAAGACAAA
GAAAGGCTAAAGAATTCCATGAGGTCAAAGAACTATGACAACTAAACACAATTCTGGATGGAATATCAAATTAAAAAA
TAACAGATAAATAATATTATTGGGAAAGTTGAATAAATTTGAATATGGACTGTTTATTAGTTATTAGTATTATAATAG
TGTTAATTTTCCTAATTTTGTTAAGACTAGTGTGCCTGTTCCATGAAAATAGAAAATGTTCTTATTCTCTGAAAATGC
ATGCTAAAGTATTTAGGGGTGAATGCAACAATGTCTGCAGCTCATTCTTGAATCAGTTCAAAGAAAAATGAGTTACAT
TTATATATATATGTATGTATGTAAACAGACATAGATAAAAGTATAGATGTGTGTGTGTCTTTAGAAAGGGGAGGATTT
TTTTTTTTTTTTTGCTGTGTGTTACTGAAGTGCCTATGTCTGCGTGTTCACACTATCATATTTTGTATGCCCTGGACT
TTATAATTTCTACCTTCAAAATTAGATCTACTGTTGGTAATTAATTCAATATATACTGGTTTTTTAACTACTATTCTC
ATTTCCTAGCAGTAATCTTCCTGAAAAGTCACAGAAATGATTACATTCCTTGTTCTTCATAATAATCACTGTTTAATT
AAAATAAGAATATTTTAGAAAAGATCTGCGGCATAGTGGTTAAGACCCCAGTATTTGATGCTAAACAGATCTGATTTG
GATAACAGAAGGTGGCACTTTGCTGTTTAAGCTGGGGACCAGACACTGTGGGTATAAATAGTAATTCCAAACACAGCT
CCACAGAGCAGCACCCTTATGACAAGGTTTTCATATGTCTATAGTTAAGCCAGAAAATTAAGAATAATGCCATAAATA
TTTATAAAGCTGAACATATCCAAGTTAAAGACCTTTATCCTGAAATTGTATCTTTTAGATTATTTTCTAAAGACTAAT
ACCATTTAATGTTTAAATGTTCTTTGGAAATGATGGTGAGAATACGTGATAATGGGTCATTGGTTTTAATATTTTATT
TAGCCAAGTGGAAAATTGGCAACCTGGTGTCGGTCCTCCCATTTGTATTTTACTGGTGCATGAAATCCAAAAGTCTAG
TAACCATTGGGACAGACAACTCTACTGCATAAGTTTGTATGTTTGTATATCTGTATCACAAAGCCCAGACACTCGAAC
TATATAAACTTGTCGCACTAAAGACAGCAAATATGTCTGGTAATTGCATATTCTTCATGTGTGCACTGGAATTTCTTA
TTATATAAGAAAATAAATGTGTTTCTAAACCACCATGAATTGGGTACTGCTGATAGTACTATTCTTCCTGGCACATGG
AAATATCCCACTGAGGTTGTCAATCAATATTGATTTAATCAGTGTGGCAGCAAAGGCACTAGAGGAGGAAAATACTCT
AGACAATCAAGTATAGATGGGAAAATTGCATCACCAAGAAATTCTTAAAATCACAAGTGGATAATTTGCAATATCAAT
TGATTTTACTCAACTGCTTGAGTCCTTTATCATTCCGTTAGAACACTCCTCATATTTGCAGATAACTCAGCCCCTCAC
CTCAGCAACAAGCAAGAGGACATCATTATAAACTTCCTCAATTTCTTTCCTATCCAGTGAAAAATATCTAAGTCCCTT
TTCCCTTCCCTTTTCCTTCCCTTCCCTTCCTTTTTCTTTCCTTCCATTCTCTTCCTTTTTCCTTCCTTTTTCTTTCCT
TCCATTCTCTTCCTTTTTCCTTCCTTTTTCTTACTTTACCTCCCCCTCCTACCTTTCCCTTTCTCCCTTCCTCCCTCT
CTCCCTTCCTTCCTCTCTCCCTCTCCTTCCTTCCTTCCTTCAATCCATACTTTTATATCTTTTCAGAGATCAATTTTT
CCCATTCATTTCTACATTCTCAGATACCTTGCTCCATGACTTGTTCTACTTCTCTTCTATCTTTATTCTTTCCCTCTT
GACTGATCCTTTTTGTCTGCCTTTGCATTTTCAAAATCAGTTTTTCTAATCTTAAAAAATAACTTTGCTCAACCCTGA
TGCATCTTTATTCACTGTGTCATTATCTCATTATTTTTAAAGGTTAGTTTCTAAAGCCAGGGCTCTATACTCACTACC
TTCACTCTTGGTTAAAATTAAAAGAAATATAGGTCCCAGTGATATTTATGGGAAAATTTGACTGTGAATAATCTTGTT
GATATAAACACTTTGGGTTTTCAAAATGTTATGCTAGCTGTTAACTTTCCCAGTAACATTATGAGGGAAGGGCTGGAA
AATACAAAAGGTAGACATAGGAACCGAGAACACCACCCCTATGGAGCCAAGCTATACATACTCTGTCTCTGGTTATCA
TAAATACATCTCCTATATTCAATGCTTGCTTAGAAAGGCATGATGTCCACGGAGCAATTCAATTGTTCACTGTGATGG
GTTTTTTTGTTTTCTCCTCATGGAATGCAACTTTTATTTAT
SEQ ID NO: 14 >NM_001165964.3 SCN1A [organism = Homo sapiens] [GeneID = 6323]
[transcript = 3]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGCAGGCAGCAACGGCAACTGCCTCAGA
ACATTCCAGAGAGCCCAGTGCAGCAGGCAGGCTCTCAGACAGCTCATCTGAAGCCTCTAAGTTGAGTTCCAAGAGTGC
TAAGGAAAGAAGAAATCGGAGGAAGAAAAGAAAACAGAAAGAGCAGTCTGGTGGGGAAGAGAAAGATGAGGATGAATT
CCAAAAATCTGAATCTGAGGACAGCATCAGGAGGAAAGGTTTTCGCTTCTCCATTGAAGGGAACCGATTGACATATGA
AAAGAGGTACTCCTCCCCACACCAGTCTTTGTTGAGCATCCGTGGCTCCCTATTTTCACCAAGGCGAAATAGCAGAAC
AAGCCTTTTCAGCTTTAGAGGGCGAGCAAAGGATGTGGGATCTGAGAACGACTTCGCAGATGATGAGCACAGCACCTT
TGAGGATAACGAGAGCCGTAGAGATTCCTTGTTTGTGCCCCGACGACACGGAGAGAGACGCAACAGCAACCTGAGTCA
GACCAGTAGGTCATCCCGGATGCTGGCAGTGTTTCCAGCGAATGGGAAGATGCACAGCACTGTGGATTGCAATGGTGT
GGTTTCCTTGGGAACAACCACTGAAACTGAAATGAGAAAGAGAAGGTCAAGTTCTTTCCACGTTTCCATGGACTTTCT
AGAAGATCCTTCCCAAAGGCAACGAGCAATGAGTATAGCCAGCATTCTAACAAATACAGTAGAAGAACTTGAAGAATC
CAGGCAGAAATGCCCACCCTGTTGGTATAAATTTTCCAACATATTCTTAATCTGGGACTGTTCTCCATATTGGTTAAA
AGTGAAACATGTTGTCAACCTGGTTGTGATGGACCCATTTGTTGACCTGGCCATCACCATCTGTATTGTCTTAAATAC
TCTTTTCATGGCCATGGAGCACTATCCAATGACGGACCATTTCAATAATGTGCTTACAGTAGGAAACTTGGTTTTCAC
TGGGATCTTTACAGCAGAAATGTTTCTGAAAATTATTGCCATGGATCCTTACTATTATTTCCAAGAAGGCTGGAATAT
CTTTGACGGTTTTATTGTGACGCTTAGCCTGGTAGAACTTGGACTCGCCAATGTGGAAGGATTATCTGTTCTCCGTTC
ATTTCGATTGCTGCGAGTTTTCAAGTTGGCAAAATCTTGGCCAACGTTAAATATGCTAATAAAGATCATCGGCAATTC
CGTGGGGGCTCTGGGAAATTTAACCCTCGTCTTGGCCATCATCGTCTTCATTTTTGCCGTGGTCGGCATGCAGCTCTT
TGGTAAAAGCTACAAAGATTGTGTCTGCAAGATCGCCAGTGATTGTCAACTCCCACGCTGGCACATGAATGACTTCTT
CCACTCCTTCCTGATTGTGTTCCGCGTGCTGTGTGGGGAGTGGATAGAGACCATGTGGGACTGTATGGAGGTTGCTGG
TCAAGCCATGTGCCTTACTGTCTTCATGATGGTCATGGTGATTGGAAACCTAGTGGTCCTGAATCTCTTTCTGGCCTT
GCTTCTGAGCTCATTTAGTGCAGACAACCTTGCAGCCACTGATGATGATAATGAAATGAATAATCTCCAAATTGCTGT
GGATAGGATGCACAAAGGAGTAGCTTATGTGAAAAGAAAAATATATGAATTTATTCAACAGTCCTTCATTAGGAAACA
AAAGATTTTAGATGAAATTAAACCACTTGATGATCTAAACAACAAGAAAGACAGTTGTATGTCCAATCATACAGCAGA
AATTGGGAAAGATCTTGACTATCTTAAAGATGTAAATGGAACTACAAGTGGTATAGGAACTGGCAGCAGTGTTGAAAA
ATACATTATTGATGAAAGTGATTACATGTCATTCATAAACAACCCCAGTCTTACTGTGACTGTACCAATTGCTGTAGG
AGAATCTGACTTTGAAAATTTAAACACGGAAGACTTTAGTAGTGAATCGGATCTGGAAGAAAGCAAAGAGAAACTGAA
TGAAAGCAGTAGCTCATCAGAAGGTAGCACTGTGGACATCGGCGCACCTGTAGAAGAACAGCCCGTAGTGGAACCTGA
AGAAACTCTTGAACCAGAAGCTTGTTTCACTGAAGGCTGTGTACAAAGATTCAAGTGTTGTCAAATCAATGTGGAAGA
AGGCAGAGGAAAACAATGGTGGAACCTGAGAAGGACGTGTTTCCGAATAGTTGAACATAACTGGTTTGAGACCTTCAT
TGTTTTCATGATTCTCCTTAGTAGTGGTGCTCTGGCATTTGAAGATATATATATTGATCAGCGAAAGACGATTAAGAC
GATGTTGGAATATGCTGACAAGGTTTTCACTTACATTTTCATTCTGGAAATGCTTCTAAAATGGGTGGCATATGGCTA
TCAAACATATTTCACCAATGCCTGGTGTTGGCTGGACTTCTTAATTGTTGATGTTTCATTGGTCAGTTTAACAGCAAA
TGCCTTGGGTTACTCAGAACTTGGAGCCATCAAATCTCTCAGGACACTAAGAGCTCTGAGACCTCTAAGAGCCTTATC
TCGATTTGAAGGGATGAGGGTGGTTGTGAATGCCCTTTTAGGAGCAATTCCATCCATCATGAATGTGCTTCTGGTTTG
TCTTATATTCTGGCTAATTTTCAGCATCATGGGCGTAAATTTGTTTGCTGGCAAATTCTACCACTGTATTAACACCAC
AACTGGTGACAGGTTTGACATCGAAGACGTGAATAATCATACTGATTGCCTAAAACTAATAGAAAGAAATGAGACTGC
TCGATGGAAAAATGTGAAAGTAAACTTTGATAATGTAGGATTTGGGTATCTCTCTTTGCTTCAAGTTGCCACATTCAA
AGGATGGATGGATATAATGTATGCAGCAGTTGATTCCAGAAATGTGGAACTCCAGCCTAAGTATGAAGAAAGTCTGTA
CATGTATCTTTACTTTGTTATTTTCATCATCTTTGGGTCCTTCTTCACCTTGAACCTGTTTATTGGTGTCATCATAGA
TAATTTCAACCAGCAGAAAAAGAAGTTTGGAGGTCAAGACATCTTTATGACAGAAGAACAGAAGAAATACTATAATGC
AATGAAAAAATTAGGATCGAAAAAACCGCAAAAGCCTATACCTCGACCAGGAAACAAATTTCAAGGAATGGTCTTTGA
CTTCGTAACCAGACAAGTTTTTGACATAAGCATCATGATTCTCATCTGTCTTAACATGGTCACAATGATGGTGGAAAC
AGATGACCAGAGTGAATATGTGACTACCATTTTGTCACGCATCAATCTGGTGTTCATTGTGCTATTTACTGGAGAGTG
TGTACTGAAACTCATCTCTCTACGCCATTATTATTTTACCATTGGATGGAATATTTTTGATTTTGTGGTTGTCATTCT
CTCCATTGTAGGTATGTTTCTTGCCGAGCTGATAGAAAAGTATTTCGTGTCCCCTACCCTGTTCCGAGTGATCCGTCT
TGCTAGGATTGGCCGAATCCTACGTCTGATCAAAGGAGCAAAGGGGATCCGCACGCTGCTCTTTGCTTTGATGATGTC
CCTTCCTGCGTTGTTTAACATCGGCCTCCTACTCTTCCTAGTCATGTTCATCTACGCCATCTTTGGGATGTCCAACTT
TGCCTATGTTAAGAGGGAAGTTGGGATCGATGACATGTTCAACTTTGAGACCTTTGGCAACAGCATGATCTGCCTATT
CCAAATTACAACCTCTGCTGGCTGGGATGGATTGCTAGCACCCATTCTCAACAGTAAGCCACCCGACTGTGACCCTAA
TAAAGTTAACCCTGGAAGCTCAGTTAAGGGAGACTGTGGGAACCCATCTGTTGGAATTTTCTTTTTTGTCAGTTACAT
CATCATATCCTTCCTGGTTGTGGTGAACATGTACATCGCGGTCATCCTGGAGAACTTCAGTGTTGCTACTGAAGAAAG
TGCAGAGCCTCTGAGTGAGGATGACTTTGAGATGTTCTATGAGGTTTGGGAGAAGTTTGATCCCGATGCAACTCAGTT
CATGGAATTTGAAAAATTATCTCAGTTTGCAGCTGCGCTTGAACCGCCTCTCAATCTGCCACAACCAAACAAACTCCA
GCTCATTGCCATGGATTTGCCCATGGTGAGTGGTGACCGGATCCACTGTCTTGATATCTTATTTGCTTTTACAAAGCG
GGTTCTAGGAGAGAGTGGAGAGATGGATGCTCTACGAATACAGATGGAAGAGCGATTCATGGCTTCCAATCCTTCCAA
GGTCTCCTATCAGCCAATCACTACTACTTTAAAACGAAAACAAGAGGAAGTATCTGCTGTCATTATTCAGCGTGCTTA
CAGACGCCACCTTTTAAAGCGAACTGTAAAACAAGCTTCCTTTACGTACAATAAAAACAAAATCAAAGGTGGGGCTAA
TCTTCTTATAAAAGAAGACATGATAATTGACAGAATAAATGAAAACTCTATTACAGAAAAAACTGATCTGACCATGTC
CACTGCAGCTTGTCCACCTTCCTATGACCGGGTGACAAAGCCAATTGTGGAAAAACATGAGCAAGAAGGCAAAGATGA
AAAAGCCAAAGGGAAATAAATGAAAATAAATAAAAATAATTGGGTGACAAATTGTTTACAGCCTGTGAAGGTGATGTA
TTTTTATCAACAGGACTCCTTTAGGAGGTCAATGCCAAACTGACTGTTTTTACACAAATCTCCTTAAGGTCAGTGCCT
ACAATAAGACAGTGACCCCTTGTCAGCAAACTGTGACTCTGTGTAAAGGGGAGATGACCTTGACAGGAGGTTACTGTT
CTCACTACCAGCTGACACTGCTGAAGATAAGATGCACAATGGCTAGTCAGACTGTAGGGACCAGTTTCAAGGGGTGCA
AACCTGTGATTTTGGGGTTGTTTAACATGAAACACTTTAGTGTAGTAATTGTATCCACTGTTTGCATTTCAACTGCCA
CATTTGTCACATTTTTATGGAATCTGTTAGTGGATTCATCTTTTTGTTAATCCATGTGTTTATTATATGTGACTATTT
TTGTAAACGAAGTTTCTGTTGAGAAATAGGCTAAGGACCTCTATAACAGGTATGCCACCTGGGGGGTATGGCAACCAC
ATGGCCCTCCCAGCTACACAAAGTCGTGGTTTGCATGAGGGCATGCTGCACTTAGAGATCATGCATGAGAAAAAGTCA
CAAGAAAAACAAATTCTTAAATTTCACCATATTTCTGGGAGGGGTAATTGGGTGATAAGTGGAGGTGCTTTGTTGATC
TTGTTTTGCGAAATCCAGCCCCTAGACCAAGTAGATTATTTGTGGGTAGGCCAGTAAATCTTAGCAGGTGCAAACTTC
ATTCAAATGTTTGGAGTCATAAATGTTATGTTTCTTTTTGTTGTATTAAAAAAAAAACCTGAATAGTGAATATTGCCC
CTCACCCTCCACCGCCAGAAGACTGAATTGACCAAAATTACTCTTTATAAATTTCTGCTTTTTCCTGCACTTTGTTTA
GCCATCTTCGGCTCTCAGCAAGGTTGACACTGTATATGTTAATGAAATGCTATTTATTATGTAAATAGTCATTTTACC
CTGTGGTGCACGTTTGAGCAAACAAATAATGACCTAAGCACAGTATTTATTGCATCAAATATGTACCACAAGAAATGT
AGAGTGCAAGCTTTACACAGGTAATAAAATGTATTCTGTACCATTTATAGATAGTTTGGATGCTATCAATGCATGTTT
ATATTACCATGCTGCTGTATCTGGTTTCTCTCACTGCTCAGAATCTCATTTATGAGAAACCATATGTCAGTGGTAAAG
TCAAGGAAATTGTTCAACAGATCTCATTTATTTAAGTCATTAAGCAATAGTTTGCAGCACTTTAACAGCTTTTTGGTT
ATTTTTACATTTTAAGTGGATAACATATGGTATATAGCCAGACTGTACAGACATGTTTAAAAAAACACACTGCTTAAC
CTATTAAATATGTGTTTAGAATTTTATAAGCAAATATAAATACTGTAAAAAGTCACTTTATTTTATTTTTCAGCATTA
TGTACATAAATATGAAGAGGAAATTATCTTCAGGTTGATATCACAATCACTTTTCTTACTTTCTGTCCATAGTACTTT
TTCATGAAAGAAATTTGCTAAATAAGACATGAAAACAAGACTGGGTAGTTGTAGATTTCTGCTTTTTAAATTACATTT
GCTAATTTTAGATTATTTCACAATTTTAAGGAGCAAAATAGGTTCACGATTCATATCCAAATTATGCTTTGCAATTGG
AAAAGGGTTTAAAATTTTATTTATATTTCTGGTAGTACCTGCACTAACTGAATTGAAGGTAGTGCTTATGTTATTTTT
GTTCTTTTTTTCTGACTTCGGTTTATGTTTTCATTTCTTTGGAGTAATGCTGCTCTAGATTGTTCTAAATAGAATGTG
GGCTTCATAATTTTTTTTTCCACAAAAACAGAGTAGTCAACTTATATAGTCAATTACATCAGGACATTTTGTGTTTCT
TACAGAAGCAAACCATAGGCTCCTCTTTTCCTTAAAACTACTTAGATAAACTGTATTCGTGAACTGCATGCTGGAAAA
TGCTACTATTATGCTAAATAATGCTAACCAACATTTAAAATGTGCAAAACTAATAAAGATTACATTTTTTATTTTATT
GTTTGCCCAGTCACTTTTTGTTAACAGAATATTCTAATGATATGGAGATTTTTTACATTACAAATTGGGGGAGAAGGG
GAGCGCGCGCGCACACACACACACACACACACACACACACACACACACACAGAGGCATACCCACGTTGACAACAAAAC
CTAGGGTAGATATGTCACTGGAGGTAGGGGGTAATGACCTCCCAGAATTACAAGCAGCAGGTGTGTTCTCTGTTAGGA
GGAAGAACTGGTGTCAGAGGATAGCTAGTGATTCTAGGAGGAAGAGAAGTATGGAAGCCAGAGTGATGGTGGATGACC
CCTTGAGCTATGAAAAGAAACCCTTAAATCATCATTTAAAAATTTAGAATTGCCATGTGTGTAGGATACTGTGTTTGC
TCCTCCAGAGCCACTCTCTCTGCTTCTGCATCATTCTGTGTGTCCCAGAAGGGTGACTTCTACACATTGCAAAAATGG
GCTCTCCTACCTTTGAGCTCCCAATTGGTTTGGCCAATGAGAAGCACCAGTGGGAAAGCACCAGAGAGAGAAGATTGA
CATAGGAATATTTCTTCTCCAATTCCTTCTTTGCTGGGTTGGCACTGGACTCATTCCTCCCCGAAAAGTCATACTCCA
ATCAGACTGCCCCTCATACAACTGAAGCTACTTTCTCTGGGGTCAGGTAATCACTCCTCCCCTTGCTCCTTCAGGTCT
GCTGCTGCATTGAGAGTGCTTTTGTATTCCTTGTAGCTTTCTCCTAACATTGCTGACACTTTTGTAAATGTCCCCTTC
ATGAAATTCTTCTATATGCCTCATTTCAGCATGCCATCTGTCTCCTGCCTGGCTGACACAAGGTGATTCAACAGCTCA
TGAAAGTCAGCAGGAAGCAAAGATGTGCCTTGCTTCAGCTTGGGGTCTTAATCTTGCTAACTTTTGCAGATAAAGAAA
AACAGTAACTGGGGGAACCACAGTGAAGTCCAGTGCAGAATTCACAGATATCATGGAAAGGTTACTCGGGTGGTCCAG
ATAGTAAAATTAACAGTCTAAATTAATCTATCTAAATTTCTGAGGAACGAGAAGCCTTCCCTTGTCATCAGGTGAAGC
CAGAAGAGGGAATATAGCCTCAACCAGAAAAGGGACAGTAATTAAAAGGCTTTTCCCATCCTTGTACAATGGACTGAC
TTTGCCTCTTCATAACATCACAATCCTAAAGCAACACAACAATTAATTCTGATATATTAGTAGCTGAAAAAAATTCCC
ATTTCCAACTAAGGTAGGTCAGAATTATAGGATAAACCCTGCAGACTTTTTATACTACCCATCCACGCCATTACTCAC
TGTTACCTTTCCAAATACAAAGAGAAGAACTGGTAAAACATAATCATATAAATCTCCATATTCATTTTGAAATATTTG
GCATGATATTTTCTGTGCTAAAAAGTAATTATTCTTCAAAGAATGATGAGGTCATGTCAGTAAGACACAGGAACCAAC
TAGAAGGGGCTTCCCACTGGCCAAATCTGGGGCAAGTTGAGCATCAAAATAAATGATAGTAAAAGATTATAATTCATT
GAATAAGAATCAGCAAATACATACTGATGTAAGTAAATAAGGAAAAGTACAAATCTGTTTCTTGCAGTTGAATGTTAA
TTAACAATTGTAGAAGAAATAACGGAGTTAGAAAAATCACTATTTGGCAATCACCCTAATGACAATTGATTCATACAA
GAATCATCAATGAGTATTAAAACTCATGGGTGAAAGTTTGATGAGGAATAGGGTATTTATAGCATCTTAAAGTATCTC
TTCTCTATTAAGTAGAAAATTTAAACAGAAGAAAGTATACTTTGGAGAAATACAGCAGACAATACCTTCAAAGATATC
ATCAATTATGAGACCAACTGATACTATGTGCCTCCTGATAAGATATACTGAAAGGGCCACATTACTTCTTGGTACACA
GTCAAAATTTTAAAACCAGAATCTAACTACAAGGAAAATCAAATTGAGGACACTCTATAAAATAAGTGGACTGAACTC
CTTAAAAATGTCAATGTCATGAAAGACAAAGAAAGGCTAAAGAATTCCATGAGGTCAAAGAACTATGACAACTAAACA
CAATTCTGGATGGAATATCAAATTAAAAAATAACAGATAAATAATATTATTGGGAAAGTTGAATAAATTTGAATATGG
ACTGTTTATTAGTTATTAGTATTATAATAGTGTTAATTTTCCTAATTTTGTTAAGACTAGTGTGCCTGTTCCATGAAA
ATAGAAAATGTTCTTATTCTCTGAAAATGCATGCTAAAGTATTTAGGGGTGAATGCAACAATGTCTGCAGCTCATTCT
TGAATCAGTTCAAAGAAAAATGAGTTACATTTATATATATATGTATGTATGTAAACAGACATAGATAAAAGTATAGAT
GTGTGTGTGTCTTTAGAAAGGGGAGGATTTTTTTTTTTTTTTTGCTGTGTGTTACTGAAGTGCCTATGTCTGCGTGTT
CACACTATCATATTTTGTATGCCCTGGACTTTATAATTTCTACCTTCAAAATTAGATCTACTGTTGGTAATTAATTCA
ATATATACTGGTTTTTTAACTACTATTCTCATTTCCTAGCAGTAATCTTCCTGAAAAGTCACAGAAATGATTACATTC
CTTGTTCTTCATAATAATCACTGTTTAATTAAAATAAGAATATTTTAGAAAAGATCTGCGGCATAGTGGTTAAGACCC
CAGTATTTGATGCTAAACAGATCTGATTTGGATAACAGAAGGTGGCACTTTGCTGTTTAAGCTGGGGACCAGACACTG
TGGGTATAAATAGTAATTCCAAACACAGCTCCACAGAGCAGCACCCTTATGACAAGGTTTTCATATGTCTATAGTTAA
GCCAGAAAATTAAGAATAATGCCATAAATATTTATAAAGCTGAACATATCCAAGTTAAAGACCTTTATCCTGAAATTG
TATCTTTTAGATTATTTTCTAAAGACTAATACCATTTAATGTTTAAATGTTCTTTGGAAATGATGGTGAGAATACGTG
ATAATGGGTCATTGGTTTTAATATTTTATTTAGCCAAGTGGAAAATTGGCAACCTGGTGTCGGTCCTCCCATTTGTAT
TTTACTGGTGCATGAAATCCAAAAGTCTAGTAACCATTGGGACAGACAACTCTACTGCATAAGTTTGTATGTTTGTAT
ATCTGTATCACAAAGCCCAGACACTCGAACTATATAAACTTGTCGCACTAAAGACAGCAAATATGTCTGGTAATTGCA
TATTCTTCATGTGTGCACTGGAATTTCTTATTATATAAGAAAATAAATGTGTTTCTAAACCACCATGAATTGGGTACT
GCTGATAGTACTATTCTTCCTGGCACATGGAAATATCCCACTGAGGTTGTCAATCAATATTGATTTAATCAGTGTGGC
AGCAAAGGCACTAGAGGAGGAAAATACTCTAGACAATCAAGTATAGATGGGAAAATTGCATCACCAAGAAATTCTTAA
AATCACAAGTGGATAATTTGCAATATCAATTGATTTTACTCAACTGCTTGAGTCCTTTATCATTCCGTTAGAACACTC
CTCATATTTGCAGATAACTCAGCCCCTCACCTCAGCAACAAGCAAGAGGACATCATTATAAACTTCCTCAATTTCTTT
CCTATCCAGTGAAAAATATCTAAGTCCCTTTTCCCTTCCCTTTTCCTTCCCTTCCCTTCCTTTTTCTTTCCTTCCATT
CTCTTCCTTTTTCCTTCCTTTTTCTTTCCTTCCATTCTCTTCCTTTTTCCTTCCTTTTTCTTACTTTACCTCCCCCTC
CTACCTTTCCCTTTCTCCCTTCCTCCCTCTCTCCCTTCCTTCCTCTCTCCCTCTCCTTCCTTCCTTCCTTCAATCCAT
ACTTTTATATCTTTTCAGAGATCAATTTTTCCCATTCATTTCTACATTCTCAGATACCTTGCTCCATGACTTGTTCTA
CTTCTCTTCTATCTTTATTCTTTCCCTCTTGACTGATCCTTTTTGTCTGCCTTTGCATTTTCAAAATCAGTTTTTCTA
ATCTTAAAAAATAACTTTGCTCAACCCTGATGCATCTTTATTCACTGTGTCATTATCTCATTATTTTTAAAGGTTAGT
TTCTAAAGCCAGGGCTCTATACTCACTACCTTCACTCTTGGTTAAAATTAAAAGAAATATAGGTCCCAGTGATATTTA
TGGGAAAATTTGACTGTGAATAATCTTGTTGATATAAACACTTTGGGTTTTCAAAATGTTATGCTAGCTGTTAACTTT
CCCAGTAACATTATGAGGGAAGGGCTGGAAAATACAAAAGGTAGACATAGGAACCGAGAACACCACCCCTATGGAGCC
AAGCTATACATACTCTGTCTCTGGTTATCATAAATACATCTCCTATATTCAATGCTTGCTTAGAAAGGCATGATGTCC
ACGGAGCAATTCAATTGTTCACTGTGATGGGTTTTTTTGTTTTCTCCTCATGGAATGCAACTTTTATTTAT
SEQ ID NO: 15 >NM_001353952.2 SCN1A [organism = Homo sapiens] [GeneID = 6323]
[transcript = 9]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGCAGGCAGCAACGGCAACTGCCTCAGA
ACATTCCAGAGAGCCCAGTGCAGCAGGCAGGCTCTCAGACAGCTCATCTGAAGCCTCTAAGTTGAGTTCCAAGAGTGC
TAAGGAAAGAAGAAATCGGAGGAAGAAAAGAAAACAGAAAGAGCAGTCTGGTGGGGAAGAGAAAGATGAGGATGAATT
CCAAAAATCTGAATCTGAGGACAGCATCAGGAGGAAAGGTTTTCGCTTCTCCATTGAAGGGAACCGATTGACATATGA
AAAGAGGTACTCCTCCCCACACCAGTCTTTGTTGAGCATCCGTGGCTCCCTATTTTCACCAAGGCGAAATAGCAGAAC
AAGCCTTTTCAGCTTTAGAGGGCGAGCAAAGGATGTGGGATCTGAGAACGACTTCGCAGATGATGAGCACAGCACCTT
TGAGGATAACGAGAGCCGTAGAGATTCCTTGTTTGTGCCCCGACGACACGGAGAGAGACGCAACAGCAACCTGAGTCA
GACCAGTAGGTCATCCCGGATGCTGGCAGTGTTTCCAGCGAATGGGAAGATGCACAGCACTGTGGATTGCAATGGTGT
GGTTTCCTTGGTTGGTGGACCTTCAGTTCCTACATCGCCTGTTGGACAGCTTCTGCCAGAGGGAACAACCACTGAAAC
TGAAATGAGAAAGAGAAGGTCAAGTTCTTTCCACGTTTCCATGGACTTTCTAGAAGATCCTTCCCAAAGGCAACGAGC
AATGAGTATAGCCAGCATTCTAACAAATACAGTAGAAGAACTTGAAGAATCCAGGCAGAAATGCCCACCCTGTTGGTA
TAAATTTTCCAACATATTCTTAATCTGGGACTGTTCTCCATATTGGTTAAAAGTGAAACATGTTGTCAACCTGGTTGT
GATGGACCCATTTGTTGACCTGGCCATCACCATCTGTATTGTCTTAAATACTCTTTTCATGGCCATGGAGCACTATCC
AATGACGGACCATTTCAATAATGTGCTTACAGTAGGAAACTTGGTTTTCACTGGGATCTTTACAGCAGAAATGTTTCT
GAAAATTATTGCCATGGATCCTTACTATTATTTCCAAGAAGGCTGGAATATCTTTGACGGTTTTATTGTGACGCTTAG
CCTGGTAGAACTTGGACTCGCCAATGTGGAAGGATTATCTGTTCTCCGTTCATTTCGATTGCTGCGAGTTTTCAAGTT
GGCAAAATCTTGGCCAACGTTAAATATGCTAATAAAGATCATCGGCAATTCCGTGGGGGCTCTGGGAAATTTAACCCT
CGTCTTGGCCATCATCGTCTTCATTTTTGCCGTGGTCGGCATGCAGCTCTTTGGTAAAAGCTACAAAGATTGTGTCTG
CAAGATCGCCAGTGATTGTCAACTCCCACGCTGGCACATGAATGACTTCTTCCACTCCTTCCTGATTGTGTTCCGCGT
GCTGTGTGGGGAGTGGATAGAGACCATGTGGGACTGTATGGAGGTTGCTGGTCAAGCCATGTGCCTTACTGTCTTCAT
GATGGTCATGGTGATTGGAAACCTAGTGGTCCTGAATCTCTTTCTGGCCTTGCTTCTGAGCTCATTTAGTGCAGACAA
CCTTGCAGCCACTGATGATGATAATGAAATGAATAATCTCCAAATTGCTGTGGATAGGATGCACAAAGGAGTAGCTTA
TGTGAAAAGAAAAATATATGAATTTATTCAACAGTCCTTCATTAGGAAACAAAAGATTTTAGATGAAATTAAACCACT
TGATGATCTAAACAACAAGAAAGACAGTTGTATGTCCAATCATACAGCAGAAATTGGGAAAGATCTTGACTATCTTAA
AGATGTAAATGGAACTACAAGTGGTATAGGAACTGGCAGCAGTGTTGAAAAATACATTATTGATGAAAGTGATTACAT
GTCATTCATAAACAACCCCAGTCTTACTGTGACTGTACCAATTGCTGTAGGAGAATCTGACTTTGAAAATTTAAACAC
GGAAGACTTTAGTAGTGAATCGGATCTGGAAGAAAGCAAAGAGAAACTGAATGAAAGCAGTAGCTCATCAGAAGGTAG
CACTGTGGACATCGGCGCACCTGTAGAAGAACAGCCCGTAGTGGAACCTGAAGAAACTCTTGAACCAGAAGCTTGTTT
CACTGAAGGCTGTGTACAAAGATTCAAGTGTTGTCAAATCAATGTGGAAGAAGGCAGAGGAAAACAATGGTGGAACCT
GAGAAGGACGTGTTTCCGAATAGTTGAACATAACTGGTTTGAGACCTTCATTGTTTTCATGATTCTCCTTAGTAGTGG
TGCTCTGGCATTTGAAGATATATATATTGATCAGCGAAAGACGATTAAGACGATGTTGGAATATGCTGACAAGGTTTT
CACTTACATTTTCATTCTGGAAATGCTTCTAAAATGGGTGGCATATGGCTATCAAACATATTTCACCAATGCCTGGTG
TTGGCTGGACTTCTTAATTGTTGATGTTTCATTGGTCAGTTTAACAGCAAATGCCTTGGGTTACTCAGAACTTGGAGC
CATCAAATCTCTCAGGACACTAAGAGCTCTGAGACCTCTAAGAGCCTTATCTCGATTTGAAGGGATGAGGGTGGTTGT
GAATGCCCTTTTAGGAGCAATTCCATCCATCATGAATGTGCTTCTGGTTTGTCTTATATTCTGGCTAATTTTCAGCAT
CATGGGCGTAAATTTGTTTGCTGGCAAATTCTACCACTGTATTAACACCACAACTGGTGACAGGTTTGACATCGAAGA
CGTGAATAATCATACTGATTGCCTAAAACTAATAGAAAGAAATGAGACTGCTCGATGGAAAAATGTGAAAGTAAACTT
TGATAATGTAGGATTTGGGTATCTCTCTTTGCTTCAAGTTGCCACATTCAAAGGATGGATGGATATAATGTATGCAGC
AGTTGATTCCAGAAATGTGGAACTCCAGCCTAAGTATGAAGAAAGTCTGTACATGTATCTTTACTTTGTTATTTTCAT
CATCTTTGGGTCCTTCTTCACCTTGAACCTGTTTATTGGTGTCATCATAGATAATTTCAACCAGCAGAAAAAGAAGTT
TGGAGGTCAAGACATCTTTATGACAGAAGAACAGAAGAAATACTATAATGCAATGAAAAAATTAGGATCGAAAAAACC
GCAAAAGCCTATACCTCGACCAGGAAACAAATTTCAAGGAATGGTCTTTGACTTCGTAACCAGACAAGTTTTTGACAT
AAGCATCATGATTCTCATCTGTCTTAACATGGTCACAATGATGGTGGAAACAGATGACCAGAGTGAATATGTGACTAC
CATTTTGTCACGCATCAATCTGGTGTTCATTGTGCTATTTACTGGAGAGTGTGTACTGAAACTCATCTCTCTACGCCA
TTATTATTTTACCATTGGATGGAATATTTTTGATTTTGTGGTTGTCATTCTCTCCATTGTAGGTATGTTTCTTGCCGA
GCTGATAGAAAAGTATTTCGTGTCCCCTACCCTGTTCCGAGTGATCCGTCTTGCTAGGATTGGCCGAATCCTACGTCT
GATCAAAGGAGCAAAGGGGATCCGCACGCTGCTCTTTGCTTTGATGATGTCCCTTCCTGCGTTGTTTAACATCGGCCT
CCTACTCTTCCTAGTCATGTTCATCTACGCCATCTTTGGGATGTCCAACTTTGCCTATGTTAAGAGGGAAGTTGGGAT
CGATGACATGTTCAACTTTGAGACCTTTGGCAACAGCATGATCTGCCTATTCCAAATTACAACCTCTGCTGGCTGGGA
TGGATTGCTAGCACCCATTCTCAACAGTAAGCCACCCGACTGTGACCCTAATAAAGTTAACCCTGGAAGCTCAGTTAA
GGGAGACTGTGGGAACCCATCTGTTGGAATTTTCTTTTTTGTCAGTTACATCATCATATCCTTCCTGGTTGTGGTGAA
CATGTACATCGCGGTCATCCTGGAGAACTTCAGTGTTGCTACTGAAGAAAGTGCAGAGCCTCTGAGTGAGGATGACTT
TGAGATGTTCTATGAGGTTTGGGAGAAGTTTGATCCCGATGCAACTCAGTTCATGGAATTTGAAAAATTATCTCAGTT
TGCAGCTGCGCTTGAACCGCCTCTCAATCTGCCACAACCAAACAAACTCCAGCTCATTGCCATGGATTTGCCCATGGT
GAGTGGTGACCGGATCCACTGTCTTGATATCTTATTTGCTTTTACAAAGCGGGTTCTAGGAGAGAGTGGAGAGATGGA
TGCTCTACGAATACAGATGGAAGAGCGATTCATGGCTTCCAATCCTTCCAAGGTCTCCTATCAGCCAATCACTACTAC
TTTAAAACGAAAACAAGAGGAAGTATCTGCTGTCATTATTCAGCGTGCTTACAGACGCCACCTTTTAAAGCGAACTGT
AAAACAAGCTTCCTTTACGTACAATAAAAACAAAATCAAAGGTGGGGCTAATCTTCTTATAAAAGAAGACATGATAAT
TGACAGAATAAATGAAAACTCTATTACAGAAAAAACTGATCTGACCATGTCCACTGCAGCTTGTCCACCTTCCTATGA
CCGGGTGACAAAGCCAATTGTGGAAAAACATGAGCAAGAAGGCAAAGATGAAAAAGCCAAAGGGAAATAAATGAAAAT
AAATAAAAATAATTGGGTGACAAATTGTTTACAGCCTGTGAAGGTGATGTATTTTTATCAACAGGACTCCTTTAGGAG
GTCAATGCCAAACTGACTGTTTTTACACAAATCTCCTTAAGGTCAGTGCCTACAATAAGACAGTGACCCCTTGTCAGC
AAACTGTGACTCTGTGTAAAGGGGAGATGACCTTGACAGGAGGTTACTGTTCTCACTACCAGCTGACACTGCTGAAGA
TAAGATGCACAATGGCTAGTCAGACTGTAGGGACCAGTTTCAAGGGGTGCAAACCTGTGATTTTGGGGTTGTTTAACA
TGAAACACTTTAGTGTAGTAATTGTATCCACTGTTTGCATTTCAACTGCCACATTTGTCACATTTTTATGGAATCTGT
TAGTGGATTCATCTTTTTGTTAATCCATGTGTTTATTATATGTGACTATTTTTGTAAACGAAGTTTCTGTTGAGAAAT
AGGCTAAGGACCTCTATAACAGGTATGCCACCTGGGGGGTATGGCAACCACATGGCCCTCCCAGCTACACAAAGTCGT
GGTTTGCATGAGGGCATGCTGCACTTAGAGATCATGCATGAGAAAAAGTCACAAGAAAAACAAATTCTTAAATTTCAC
CATATTTCTGGGAGGGGTAATTGGGTGATAAGTGGAGGTGCTTTGTTGATCTTGTTTTGCGAAATCCAGCCCCTAGAC
CAAGTAGATTATTTGTGGGTAGGCCAGTAAATCTTAGCAGGTGCAAACTTCATTCAAATGTTTGGAGTCATAAATGTT
ATGTTTCTTTTTGTTGTATTAAAAAAAAAACCTGAATAGTGAATATTGCCCCTCACCCTCCACCGCCAGAAGACTGAA
TTGACCAAAATTACTCTTTATAAATTTCTGCTTTTTCCTGCACTTTGTTTAGCCATCTTCGGCTCTCAGCAAGGTTGA
CACTGTATATGTTAATGAAATGCTATTTATTATGTAAATAGTCATTTTACCCTGTGGTGCACGTTTGAGCAAACAAAT
AATGACCTAAGCACAGTATTTATTGCATCAAATATGTACCACAAGAAATGTAGAGTGCAAGCTTTACACAGGTAATAA
AATGTATTCTGTACCATTTATAGATAGTTTGGATGCTATCAATGCATGTTTATATTACCATGCTGCTGTATCTGGTTT
CTCTCACTGCTCAGAATCTCATTTATGAGAAACCATATGTCAGTGGTAAAGTCAAGGAAATTGTTCAACAGATCTCAT
TTATTTAAGTCATTAAGCAATAGTTTGCAGCACTTTAACAGCTTTTTGGTTATTTTTACATTTTAAGTGGATAACATA
TGGTATATAGCCAGACTGTACAGACATGTTTAAAAAAACACACTGCTTAACCTATTAAATATGTGTTTAGAATTTTAT
AAGCAAATATAAATACTGTAAAAAGTCACTTTATTTTATTTTTCAGCATTATGTACATAAATATGAAGAGGAAATTAT
CTTCAGGTTGATATCACAATCACTTTTCTTACTTTCTGTCCATAGTACTTTTTCATGAAAGAAATTTGCTAAATAAGA
CATGAAAACAAGACTGGGTAGTTGTAGATTTCTGCTTTTTAAATTACATTTGCTAATTTTAGATTATTTCACAATTTT
AAGGAGCAAAATAGGTTCACGATTCATATCCAAATTATGCTTTGCAATTGGAAAAGGGTTTAAAATTTTATTTATATT
TCTGGTAGTACCTGCACTAACTGAATTGAAGGTAGTGCTTATGTTATTTTTGTTCTTTTTTTCTGACTTCGGTTTATG
TTTTCATTTCTTTGGAGTAATGCTGCTCTAGATTGTTCTAAATAGAATGTGGGCTTCATAATTTTTTTTTCCACAAAA
ACAGAGTAGTCAACTTATATAGTCAATTACATCAGGACATTTTGTGTTTCTTACAGAAGCAAACCATAGGCTCCTCTT
TTCCTTAAAACTACTTAGATAAACTGTATTCGTGAACTGCATGCTGGAAAATGCTACTATTATGCTAAATAATGCTAA
CCAACATTTAAAATGTGCAAAACTAATAAAGATTACATTTTTTATTTTATTGTTTGCCCAGTCACTTTTTGTTAACAG
AATATTCTAATGATATGGAGATTTTTTACATTACAAATTGGGGGAGAAGGGGAGCGCGCGCGCACACACACACACACA
CACACACACACACACACACACACAGAGGCATACCCACGTTGACAACAAAACCTAGGGTAGATATGTCACTGGAGGTAG
GGGGTAATGACCTCCCAGAATTACAAGCAGCAGGTGTGTTCTCTGTTAGGAGGAAGAACTGGTGTCAGAGGATAGCTA
GTGATTCTAGGAGGAAGAGAAGTATGGAAGCCAGAGTGATGGTGGATGACCCCTTGAGCTATGAAAAGAAACCCTTAA
ATCATCATTTAAAAATTTAGAATTGCCATGTGTGTAGGATACTGTGTTTGCTCCTCCAGAGCCACTCTCTCTGCTTCT
GCATCATTCTGTGTGTCCCAGAAGGGTGACTTCTACACATTGCAAAAATGGGCTCTCCTACCTTTGAGCTCCCAATTG
GTTTGGCCAATGAGAAGCACCAGTGGGAAAGCACCAGAGAGAGAAGATTGACATAGGAATATTTCTTCTCCAATTCCT
TCTTTGCTGGGTTGGCACTGGACTCATTCCTCCCCGAAAAGTCATACTCCAATCAGACTGCCCCTCATACAACTGAAG
CTACTTTCTCTGGGGTCAGGTAATCACTCCTCCCCTTGCTCCTTCAGGTCTGCTGCTGCATTGAGAGTGCTTTTGTAT
TCCTTGTAGCTTTCTCCTAACATTGCTGACACTTTTGTAAATGTCCCCTTCATGAAATTCTTCTATATGCCTCATTTC
AGCATGCCATCTGTCTCCTGCCTGGCTGACACAAGGTGATTCAACAGCTCATGAAAGTCAGCAGGAAGCAAAGATGTG
CCTTGCTTCAGCTTGGGGTCTTAATCTTGCTAACTTTTGCAGATAAAGAAAAACAGTAACTGGGGGAACCACAGTGAA
GTCCAGTGCAGAATTCACAGATATCATGGAAAGGTTACTCGGGTGGTCCAGATAGTAAAATTAACAGTCTAAATTAAT
CTATCTAAATTTCTGAGGAACGAGAAGCCTTCCCTTGTCATCAGGTGAAGCCAGAAGAGGGAATATAGCCTCAACCAG
AAAAGGGACAGTAATTAAAAGGCTTTTCCCATCCTTGTACAATGGACTGACTTTGCCTCTTCATAACATCACAATCCT
AAAGCAACACAACAATTAATTCTGATATATTAGTAGCTGAAAAAAATTCCCATTTCCAACTAAGGTAGGTCAGAATTA
TAGGATAAACCCTGCAGACTTTTTATACTACCCATCCACGCCATTACTCACTGTTACCTTTCCAAATACAAAGAGAAG
AACTGGTAAAACATAATCATATAAATCTCCATATTCATTTTGAAATATTTGGCATGATATTTTCTGTGCTAAAAAGTA
ATTATTCTTCAAAGAATGATGAGGTCATGTCAGTAAGACACAGGAACCAACTAGAAGGGGCTTCCCACTGGCCAAATC
TGGGGCAAGTTGAGCATCAAAATAAATGATAGTAAAAGATTATAATTCATTGAATAAGAATCAGCAAATACATACTGA
TGTAAGTAAATAAGGAAAAGTACAAATCTGTTTCTTGCAGTTGAATGTTAATTAACAATTGTAGAAGAAATAACGGAG
TTAGAAAAATCACTATTTGGCAATCACCCTAATGACAATTGATTCATACAAGAATCATCAATGAGTATTAAAACTCAT
GGGTGAAAGTTTGATGAGGAATAGGGTATTTATAGCATCTTAAAGTATCTCTTCTCTATTAAGTAGAAAATTTAAACA
GAAGAAAGTATACTTTGGAGAAATACAGCAGACAATACCTTCAAAGATATCATCAATTATGAGACCAACTGATACTAT
GTGCCTCCTGATAAGATATACTGAAAGGGCCACATTACTTCTTGGTACACAGTCAAAATTTTAAAACCAGAATCTAAC
TACAAGGAAAATCAAATTGAGGACACTCTATAAAATAAGTGGACTGAACTCCTTAAAAATGTCAATGTCATGAAAGAC
AAAGAAAGGCTAAAGAATTCCATGAGGTCAAAGAACTATGACAACTAAACACAATTCTGGATGGAATATCAAATTAAA
AAATAACAGATAAATAATATTATTGGGAAAGTTGAATAAATTTGAATATGGACTGTTTATTAGTTATTAGTATTATAA
TAGTGTTAATTTTCCTAATTTTGTTAAGACTAGTGTGCCTGTTCCATGAAAATAGAAAATGTTCTTATTCTCTGAAAA
TGCATGCTAAAGTATTTAGGGGTGAATGCAACAATGTCTGCAGCTCATTCTTGAATCAGTTCAAAGAAAAATGAGTTA
CATTTATATATATATGTATGTATGTAAACAGACATAGATAAAAGTATAGATGTGTGTGTGTCTTTAGAAAGGGGAGGA
TTTTTTTTTTTTTTTTGCTGTGTGTTACTGAAGTGCCTATGTCTGCGTGTTCACACTATCATATTTTGTATGCCCTGG
ACTTTATAATTTCTACCTTCAAAATTAGATCTACTGTTGGTAATTAATTCAATATATACTGGTTTTTTAACTACTATT
CTCATTTCCTAGCAGTAATCTTCCTGAAAAGTCACAGAAATGATTACATTCCTTGTTCTTCATAATAATCACTGTTTA
ATTAAAATAAGAATATTTTAGAAAAGATCTGCGGCATAGTGGTTAAGACCCCAGTATTTGATGCTAAACAGATCTGAT
TTGGATAACAGAAGGTGGCACTTTGCTGTTTAAGCTGGGGACCAGACACTGTGGGTATAAATAGTAATTCCAAACACA
GCTCCACAGAGCAGCACCCTTATGACAAGGTTTTCATATGTCTATAGTTAAGCCAGAAAATTAAGAATAATGCCATAA
ATATTTATAAAGCTGAACATATCCAAGTTAAAGACCTTTATCCTGAAATTGTATCTTTTAGATTATTTTCTAAAGACT
AATACCATTTAATGTTTAAATGTTCTTTGGAAATGATGGTGAGAATACGTGATAATGGGTCATTGGTTTTAATATTTT
ATTTAGCCAAGTGGAAAATTGGCAACCTGGTGTCGGTCCTCCCATTTGTATTTTACTGGTGCATGAAATCCAAAAGTC
TAGTAACCATTGGGACAGACAACTCTACTGCATAAGTTTGTATGTTTGTATATCTGTATCACAAAGCCCAGACACTCG
AACTATATAAACTTGTCGCACTAAAGACAGCAAATATGTCTGGTAATTGCATATTCTTCATGTGTGCACTGGAATTTC
TTATTATATAAGAAAATAAATGTGTTTCTAAACCACCATGAATTGGGTACTGCTGATAGTACTATTCTTCCTGGCACA
TGGAAATATCCCACTGAGGTTGTCAATCAATATTGATTTAATCAGTGTGGCAGCAAAGGCACTAGAGGAGGAAAATAC
TCTAGACAATCAAGTATAGATGGGAAAATTGCATCACCAAGAAATTCTTAAAATCACAAGTGGATAATTTGCAATATC
AATTGATTTTACTCAACTGCTTGAGTCCTTTATCATTCCGTTAGAACACTCCTCATATTTGCAGATAACTCAGCCCCT
CACCTCAGCAACAAGCAAGAGGACATCATTATAAACTTCCTCAATTTCTTTCCTATCCAGTGAAAAATATCTAAGTCC
CTTTTCCCTTCCCTTTTCCTTCCCTTCCCTTCCTTTTTCTTTCCTTCCATTCTCTTCCTTTTTCCTTCCTTTTTCTTT
CCTTCCATTCTCTTCCTTTTTCCTTCCTTTTTCTTACTTTACCTCCCCCTCCTACCTTTCCCTTTCTCCCTTCCTCCC
TCTCTCCCTTCCTTCCTCTCTCCCTCTCCTTCCTTCCTTCCTTCAATCCATACTTTTATATCTTTTCAGAGATCAATT
TTTCCCATTCATTTCTACATTCTCAGATACCTTGCTCCATGACTTGTTCTACTTCTCTTCTATCTTTATTCTTTCCCT
CTTGACTGATCCTTTTTGTCTGCCTTTGCATTTTCAAAATCAGTTTTTCTAATCTTAAAAAATAACTTTGCTCAACCC
TGATGCATCTTTATTCACTGTGTCATTATCTCATTATTTTTAAAGGTTAGTTTCTAAAGCCAGGGCTCTATACTCACT
ACCTTCACTCTTGGTTAAAATTAAAAGAAATATAGGTCCCAGTGATATTTATGGGAAAATTTGACTGTGAATAATCTT
GTTGATATAAACACTTTGGGTTTTCAAAATGTTATGCTAGCTGTTAACTTTCCCAGTAACATTATGAGGGAAGGGCTG
GAAAATACAAAAGGTAGACATAGGAACCGAGAACACCACCCCTATGGAGCCAAGCTATACATACTCTGTCTCTGGTTA
TCATAAATACATCTCCTATATTCAATGCTTGCTTAGAAAGGCATGATGTCCACGGAGCAATTCAATTGTTCACTGTGA
TGGGTTTTTTTGTTTTCTCCTCATGGAATGCAACTTTTATTTAT
SEQ ID NO: 16 >NM_001353961.2 SCN1A [organism = Homo sapiens] [GeneID = 6323]
[transcript = 15]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGGTTTGGGCAATTATGAATAAGGCTGCTGTATACATCCGTGTGCAGGATTTTGTGTGGACATA
AGTTTTCAACTCCTTTGGTTAAATCCTAAGGAATTTCATATGCAGAATAAATGGTAATTAAAATGTGCAGGATGACAA
GATGGAGCAAACAGTGCTTGTACCACCAGGACCTGACAGCTTCAACTTCTTCACCAGAGAATCTCTTGCGGCTATTGA
AAGACGCATTGCAGAAGAAAAGGCAAAGAATCCCAAACCAGACAAAAAAGATGACGACGAAAATGGCCCAAAGCCAAA
TAGTGACTTGGAAGCTGGAAAGAACCTTCCATTTATTTATGGAGACATTCCTCCAGAGATGGTGTCAGAGCCCCTGGA
GGACCTGGACCCCTACTATATCAATAAGAAAACTTTTATAGTATTGAATAAAGGGAAGGCCATCTTCCGGTTCAGTGC
CACCTCTGCCCTGTACATTTTAACTCCCTTCAATCCTCTTAGGAAAATAGCTATTAAGATTTTGGTACATTCATTATT
CAGCATGCTAATTATGTGCACTATTTTGACAAACTGTGTGTTTATGACAATGAGTAACCCTCCTGATTGGACAAAGAA
TGTAGAATACACCTTCACAGGAATATATACTTTTGAATCACTTATAAAAATTATTGCAAGGGGATTCTGTTTAGAAGA
TTTTACTTTCCTTCGGGATCCATGGAACTGGCTCGATTTCACTGTCATTACATTTGCGTACGTCACAGAGTTTGTGGA
CCTGGGCAATGTCTCGGCATTGAGAACATTCAGAGTTCTCCGAGCATTGAAGACGATTTCAGTCATTCCAGGCCTGAA
AACCATTGTGGGAGCCCTGATCCAGTCTGTGAAGAAGCTCTCAGATGTAATGATCCTGACTGTGTTCTGTCTGAGCGT
ATTTGCTCTAATTGGGCTGCAGCTGTTCATGGGCAACCTGAGGAATAAATGTATACAATGGCCTCCCACCAATGCTTC
CTTGGAGGAACATAGTATAGAAAAGAATATAACTGTGAATTATAATGGTACACTTATAAATGAAACTGTCTTTGAGTT
TGACTGGAAGTCATATATTCAAGATTCAAGATATCATTATTTCCTGGAGGGTTTTTTAGATGCACTACTATGTGGAAA
TAGCTCTGATGCAGGCCAATGTCCAGAGGGATATATGTGTGTGAAAGCTGGTAGAAATCCCAATTATGGCTACACAAG
CTTTGATACCTTCAGTTGGGCTTTTTTGTCCTTGTTTCGACTAATGACTCAGGACTTCTGGGAAAATCTTTATCAACT
GACATTACGTGCTGCTGGGAAAACGTACATGATATTTTTTGTATTGGTCATTTTCTTGGGCTCATTCTACCTAATAAA
TTTGATCCTGGCTGTGGTGGCCATGGCCTACGAGGAACAGAATCAGGCCACCTTGGAAGAAGCAGAACAGAAAGAGGC
CGAATTTCAGCAGATGATTGAACAGCTTAAAAAGCAACAGGAGGCAGCTCAGCAGGCAGCAACGGCAACTGCCTCAGA
ACATTCCAGAGAGCCCAGTGCAGCAGGCAGGCTCTCAGACAGCTCATCTGAAGCCTCTAAGTTGAGTTCCAAGAGTGC
TAAGGAAAGAAGAAATCGGAGGAAGAAAAGAAAACAGAAAGAGCAGTCTGGTGGGGAAGAGAAAGATGAGGATGAATT
CCAAAAATCTGAATCTGAGGACAGCATCAGGAGGAAAGGTTTTCGCTTCTCCATTGAAGGGAACCGATTGACATATGA
AAAGAGGTACTCCTCCCCACACCAGTCTTTGTTGAGCATCCGTGGCTCCCTATTTTCACCAAGGCGAAATAGCAGAAC
AAGCCTTTTCAGCTTTAGAGGGCGAGCAAAGGATGTGGGATCTGAGAACGACTTCGCAGATGATGAGCACAGCACCTT
TGAGGATAACGAGAGCCGTAGAGATTCCTTGTTTGTGCCCCGACGACACGGAGAGAGACGCAACAGCAACCTGAGTCA
GACCAGTAGGTCATCCCGGATGCTGGCAGTGTTTCCAGCGAATGGGAAGATGCACAGCACTGTGGATTGCAATGGTGT
GGTTTCCTTGGTTGGTGGACCTTCAGTTCCTACATCGCCTGTTGGACAGCTTCTGCCAGAGGGAACAACCACTGAAAC
TGAAATGAGAAAGAGAAGGTCAAGTTCTTTCCACGTTTCCATGGACTTTCTAGAAGATCCTTCCCAAAGGCAACGAGC
AATGAGTATAGCCAGCATTCTAACAAATACAGTAGAAGAACTTGAAGAATCCAGGCAGAAATGCCCACCCTGTTGGTA
TAAATTTTCCAACATATTCTTAATCTGGGACTGTTCTCCATATTGGTTAAAAGTGAAACATGTTGTCAACCTGGTTGT
GATGGACCCATTTGTTGACCTGGCCATCACCATCTGTATTGTCTTAAATACTCTTTTCATGGCCATGGAGCACTATCC
AATGACGGACCATTTCAATAATGTGCTTACAGTAGGAAACTTGGTAAGCATATTGGAAGGTTTTCACTGGGATCTTTA
CAGCAGAAATGTTTCTGAAAATTATTGCCATGGATCCTTACTATTATTTCCAAGAAGGCTGGAATATCTTTGACGGTT
TTATTGTGACGCTTAGCCTGGTAGAACTTGGACTCGCCAATGTGGAAGGATTATCTGTTCTCCGTTCATTTCGATTGC
TGCGAGTTTTCAAGTTGGCAAAATCTTGGCCAACGTTAAATATGCTAATAAAGATCATCGGCAATTCCGTGGGGGCTC
TGGGAAATTTAACCCTCGTCTTGGCCATCATCGTCTTCATTTTTGCCGTGGTCGGCATGCAGCTCTTTGGTAAAAGCT
ACAAAGATTGTGTCTGCAAGATCGCCAGTGATTGTCAACTCCCACGCTGGCACATGAATGACTTCTTCCACTCCTTCC
TGATTGTGTTCCGCGTGCTGTGTGGGGAGTGGATAGAGACCATGTGGGACTGTATGGAGGTTGCTGGTCAAGCCATGT
GCCTTACTGTCTTCATGATGGTCATGGTGATTGGAAACCTAGTGGTCCTGAATCTCTTTCTGGCCTTGCTTCTGAGCT
CATTTAGTGCAGACAACCTTGCAGCCACTGATGATGATAATGAAATGAATAATCTCCAAATTGCTGTGGATAGGATGC
ACAAAGGAGTAGCTTATGTGAAAAGAAAAATATATGAATTTATTCAACAGTCCTTCATTAGGAAACAAAAGATTTTAG
ATGAAATTAAACCACTTGATGATCTAAACAACAAGAAAGACAGTTGTATGTCCAATCATACAGCAGAAATTGGGAAAG
ATCTTGACTATCTTAAAGATGTAAATGGAACTACAAGTGGTATAGGAACTGGCAGCAGTGTTGAAAAATACATTATTG
ATGAAAGTGATTACATGTCATTCATAAACAACCCCAGTCTTACTGTGACTGTACCAATTGCTGTAGGAGAATCTGACT
TTGAAAATTTAAACACGGAAGACTTTAGTAGTGAATCGGATCTGGAAGAAAGCAAAGAGAAACTGAATGAAAGCAGTA
GCTCATCAGAAGGTAGCACTGTGGACATCGGCGCACCTGTAGAAGAACAGCCCGTAGTGGAACCTGAAGAAACTCTTG
AACCAGAAGCTTGTTTCACTGAAGGCTGTGTACAAAGATTCAAGTGTTGTCAAATCAATGTGGAAGAAGGCAGAGGAA
AACAATGGTGGAACCTGAGAAGGACGTGTTTCCGAATAGTTGAACATAACTGGTTTGAGACCTTCATTGTTTTCATGA
TTCTCCTTAGTAGTGGTGCTCTGGCATTTGAAGATATATATATTGATCAGCGAAAGACGATTAAGACGATGTTGGAAT
ATGCTGACAAGGTTTTCACTTACATTTTCATTCTGGAAATGCTTCTAAAATGGGTGGCATATGGCTATCAAACATATT
TCACCAATGCCTGGTGTTGGCTGGACTTCTTAATTGTTGATGTTTCATTGGTCAGTTTAACAGCAAATGCCTTGGGTT
ACTCAGAACTTGGAGCCATCAAATCTCTCAGGACACTAAGAGCTCTGAGACCTCTAAGAGCCTTATCTCGATTTGAAG
GGATGAGGGTGGTTGTGAATGCCCTTTTAGGAGCAATTCCATCCATCATGAATGTGCTTCTGGTTTGTCTTATATTCT
GGCTAATTTTCAGCATCATGGGCGTAAATTTGTTTGCTGGCAAATTCTACCACTGTATTAACACCACAACTGGTGACA
GGTTTGACATCGAAGACGTGAATAATCATACTGATTGCCTAAAACTAATAGAAAGAAATGAGACTGCTCGATGGAAAA
ATGTGAAAGTAAACTTTGATAATGTAGGATTTGGGTATCTCTCTTTGCTTCAAGTTGCCACATTCAAAGGATGGATGG
ATATAATGTATGCAGCAGTTGATTCCAGAAATGTGGAACTCCAGCCTAAGTATGAAGAAAGTCTGTACATGTATCTTT
ACTTTGTTATTTTCATCATCTTTGGGTCCTTCTTCACCTTGAACCTGTTTATTGGTGTCATCATAGATAATTTCAACC
AGCAGAAAAAGAAGTTTGGAGGTCAAGACATCTTTATGACAGAAGAACAGAAGAAATACTATAATGCAATGAAAAAAT
TAGGATCGAAAAAACCGCAAAAGCCTATACCTCGACCAGGAAACAAATTTCAAGGAATGGTCTTTGACTTCGTAACCA
GACAAGTTTTTGACATAAGCATCATGATTCTCATCTGTCTTAACATGGTCACAATGATGGTGGAAACAGATGACCAGA
GTGAATATGTGACTACCATTTTGTCACGCATCAATCTGGTGTTCATTGTGCTATTTACTGGAGAGTGTGTACTGAAAC
TCATCTCTCTACGCCATTATTATTTTACCATTGGATGGAATATTTTTGATTTTGTGGTTGTCATTCTCTCCATTGTAG
GTATGTTTCTTGCCGAGCTGATAGAAAAGTATTTCGTGTCCCCTACCCTGTTCCGAGTGATCCGTCTTGCTAGGATTG
GCCGAATCCTACGTCTGATCAAAGGAGCAAAGGGGATCCGCACGCTGCTCTTTGCTTTGATGATGTCCCTTCCTGCGT
TGTTTAACATCGGCCTCCTACTCTTCCTAGTCATGTTCATCTACGCCATCTTTGGGATGTCCAACTTTGCCTATGTTA
AGAGGGAAGTTGGGATCGATGACATGTTCAACTTTGAGACCTTTGGCAACAGCATGATCTGCCTATTCCAAATTACAA
CCTCTGCTGGCTGGGATGGATTGCTAGCACCCATTCTCAACAGTAAGCCACCCGACTGTGACCCTAATAAAGTTAACC
CTGGAAGCTCAGTTAAGGGAGACTGTGGGAACCCATCTGTTGGAATTTTCTTTTTTGTCAGTTACATCATCATATCCT
TCCTGGTTGTGGTGAACATGTACATCGCGGTCATCCTGGAGAACTTCAGTGTTGCTACTGAAGAAAGTGCAGAGCCTC
TGAGTGAGGATGACTTTGAGATGTTCTATGAGGTTTGGGAGAAGTTTGATCCCGATGCAACTCAGTTCATGGAATTTG
AAAAATTATCTCAGTTTGCAGCTGCGCTTGAACCGCCTCTCAATCTGCCACAACCAAACAAACTCCAGCTCATTGCCA
TGGATTTGCCCATGGTGAGTGGTGACCGGATCCACTGTCTTGATATCTTATTTGCTTTTACAAAGCGGGTTCTAGGAG
AGAGTGGAGAGATGGATGCTCTACGAATACAGATGGAAGAGCGATTCATGGCTTCCAATCCTTCCAAGGTCTCCTATC
AGCCAATCACTACTACTTTAAAACGAAAACAAGAGGAAGTATCTGCTGTCATTATTCAGCGTGCTTACAGACGCCACC
TTTTAAAGCGAACTGTAAAACAAGCTTCCTTTACGTACAATAAAAACAAAATCAAAGGTGGGGCTAATCTTCTTATAA
AAGAAGACATGATAATTGACAGAATAAATGAAAACTCTATTACAGAAAAAACTGATCTGACCATGTCCACTGCAGCTT
GTCCACCTTCCTATGACCGGGTGACAAAGCCAATTGTGGAAAAACATGAGCAAGAAGGCAAAGATGAAAAAGCCAAAG
GGAAATAAATGAAAATAAATAAAAATAATTGGGTGACAAATTGTTTACAGCCTGTGAAGGTGATGTATTTTTATCAAC
AGGACTCCTTTAGGAGGTCAATGCCAAACTGACTGTTTTTACACAAATCTCCTTAAGGTCAGTGCCTACAATAAGACA
GTGACCCCTTGTCAGCAAACTGTGACTCTGTGTAAAGGGGAGATGACCTTGACAGGAGGTTACTGTTCTCACTACCAG
CTGACACTGCTGAAGATAAGATGCACAATGGCTAGTCAGACTGTAGGGACCAGTTTCAAGGGGTGCAAACCTGTGATT
TTGGGGTTGTTTAACATGAAACACTTTAGTGTAGTAATTGTATCCACTGTTTGCATTTCAACTGCCACATTTGTCACA
TTTTTATGGAATCTGTTAGTGGATTCATCTTTTTGTTAATCCATGTGTTTATTATATGTGACTATTTTTGTAAACGAA
GTTTCTGTTGAGAAATAGGCTAAGGACCTCTATAACAGGTATGCCACCTGGGGGGTATGGCAACCACATGGCCCTCCC
AGCTACACAAAGTCGTGGTTTGCATGAGGGCATGCTGCACTTAGAGATCATGCATGAGAAAAAGTCACAAGAAAAACA
AATTCTTAAATTTCACCATATTTCTGGGAGGGGTAATTGGGTGATAAGTGGAGGTGCTTTGTTGATCTTGTTTTGCGA
AATCCAGCCCCTAGACCAAGTAGATTATTTGTGGGTAGGCCAGTAAATCTTAGCAGGTGCAAACTTCATTCAAATGTT
TGGAGTCATAAATGTTATGTTTCTTTTTGTTGTATTAAAAAAAAAACCTGAATAGTGAATATTGCCCCTCACCCTCCA
CCGCCAGAAGACTGAATTGACCAAAATTACTCTTTATAAATTTCTGCTTTTTCCTGCACTTTGTTTAGCCATCTTCGG
CTCTCAGCAAGGTTGACACTGTATATGTTAATGAAATGCTATTTATTATGTAAATAGTCATTTTACCCTGTGGTGCAC
GTTTGAGCAAACAAATAATGACCTAAGCACAGTATTTATTGCATCAAATATGTACCACAAGAAATGTAGAGTGCAAGC
TTTACACAGGTAATAAAATGTATTCTGTACCATTTATAGATAGTTTGGATGCTATCAATGCATGTTTATATTACCATG
CTGCTGTATCTGGTTTCTCTCACTGCTCAGAATCTCATTTATGAGAAACCATATGTCAGTGGTAAAGTCAAGGAAATT
GTTCAACAGATCTCATTTATTTAAGTCATTAAGCAATAGTTTGCAGCACTTTAACAGCTTTTTGGTTATTTTTACATT
TTAAGTGGATAACATATGGTATATAGCCAGACTGTACAGACATGTTTAAAAAAACACACTGCTTAACCTATTAAATAT
GTGTTTAGAATTTTATAAGCAAATATAAATACTGTAAAAAGTCACTTTATTTTATTTTTCAGCATTATGTACATAAAT
ATGAAGAGGAAATTATCTTCAGGTTGATATCACAATCACTTTTCTTACTTTCTGTCCATAGTACTTTTTCATGAAAGA
AATTTGCTAAATAAGACATGAAAACAAGACTGGGTAGTTGTAGATTTCTGCTTTTTAAATTACATTTGCTAATTTTAG
ATTATTTCACAATTTTAAGGAGCAAAATAGGTTCACGATTCATATCCAAATTATGCTTTGCAATTGGAAAAGGGTTTA
AAATTTTATTTATATTTCTGGTAGTACCTGCACTAACTGAATTGAAGGTAGTGCTTATGTTATTTTTGTTCTTTTTTT
CTGACTTCGGTTTATGTTTTCATTTCTTTGGAGTAATGCTGCTCTAGATTGTTCTAAATAGAATGTGGGCTTCATAAT
TTTTTTTTCCACAAAAACAGAGTAGTCAACTTATATAGTCAATTACATCAGGACATTTTGTGTTTCTTACAGAAGCAA
ACCATAGGCTCCTCTTTTCCTTAAAACTACTTAGATAAACTGTATTCGTGAACTGCATGCTGGAAAATGCTACTATTA
TGCTAAATAATGCTAACCAACATTTAAAATGTGCAAAACTAATAAAGATTACATTTTTTATTTTATTGTTTGCCCAGT
CACTTTTTGTTAACAGAATATTCTAATGATATGGAGATTTTTTACATTACAAATTGGGGGAGAAGGGGAGCGCGCGCG
CACACACACACACACACACACACACACACACACACACACAGAGGCATACCCACGTTGACAACAAAACCTAGGGTAGAT
ATGTCACTGGAGGTAGGGGGTAATGACCTCCCAGAATTACAAGCAGCAGGTGTGTTCTCTGTTAGGAGGAAGAACTGG
TGTCAGAGGATAGCTAGTGATTCTAGGAGGAAGAGAAGTATGGAAGCCAGAGTGATGGTGGATGACCCCTTGAGCTAT
GAAAAGAAACCCTTAAATCATCATTTAAAAATTTAGAATTGCCATGTGTGTAGGATACTGTGTTTGCTCCTCCAGAGC
CACTCTCTCTGCTTCTGCATCATTCTGTGTGTCCCAGAAGGGTGACTTCTACACATTGCAAAAATGGGCTCTCCTACC
TTTGAGCTCCCAATTGGTTTGGCCAATGAGAAGCACCAGTGGGAAAGCACCAGAGAGAGAAGATTGACATAGGAATAT
TTCTTCTCCAATTCCTTCTTTGCTGGGTTGGCACTGGACTCATTCCTCCCCGAAAAGTCATACTCCAATCAGACTGCC
CCTCATACAACTGAAGCTACTTTCTCTGGGGTCAGGTAATCACTCCTCCCCTTGCTCCTTCAGGTCTGCTGCTGCATT
GAGAGTGCTTTTGTATTCCTTGTAGCTTTCTCCTAACATTGCTGACACTTTTGTAAATGTCCCCTTCATGAAATTCTT
CTATATGCCTCATTTCAGCATGCCATCTGTCTCCTGCCTGGCTGACACAAGGTGATTCAACAGCTCATGAAAGTCAGC
AGGAAGCAAAGATGTGCCTTGCTTCAGCTTGGGGTCTTAATCTTGCTAACTTTTGCAGATAAAGAAAAACAGTAACTG
GGGGAACCACAGTGAAGTCCAGTGCAGAATTCACAGATATCATGGAAAGGTTACTCGGGTGGTCCAGATAGTAAAATT
AACAGTCTAAATTAATCTATCTAAATTTCTGAGGAACGAGAAGCCTTCCCTTGTCATCAGGTGAAGCCAGAAGAGGGA
ATATAGCCTCAACCAGAAAAGGGACAGTAATTAAAAGGCTTTTCCCATCCTTGTACAATGGACTGACTTTGCCTCTTC
ATAACATCACAATCCTAAAGCAACACAACAATTAATTCTGATATATTAGTAGCTGAAAAAAATTCCCATTTCCAACTA
AGGTAGGTCAGAATTATAGGATAAACCCTGCAGACTTTTTATACTACCCATCCACGCCATTACTCACTGTTACCTTTC
CAAATACAAAGAGAAGAACTGGTAAAACATAATCATATAAATCTCCATATTCATTTTGAAATATTTGGCATGATATTT
TCTGTGCTAAAAAGTAATTATTCTTCAAAGAATGATGAGGTCATGTCAGTAAGACACAGGAACCAACTAGAAGGGGCT
TCCCACTGGCCAAATCTGGGGCAAGTTGAGCATCAAAATAAATGATAGTAAAAGATTATAATTCATTGAATAAGAATC
AGCAAATACATACTGATGTAAGTAAATAAGGAAAAGTACAAATCTGTTTCTTGCAGTTGAATGTTAATTAACAATTGT
AGAAGAAATAACGGAGTTAGAAAAATCACTATTTGGCAATCACCCTAATGACAATTGATTCATACAAGAATCATCAAT
GAGTATTAAAACTCATGGGTGAAAGTTTGATGAGGAATAGGGTATTTATAGCATCTTAAAGTATCTCTTCTCTATTAA
GTAGAAAATTTAAACAGAAGAAAGTATACTTTGGAGAAATACAGCAGACAATACCTTCAAAGATATCATCAATTATGA
GACCAACTGATACTATGTGCCTCCTGATAAGATATACTGAAAGGGCCACATTACTTCTTGGTACACAGTCAAAATTTT
AAAACCAGAATCTAACTACAAGGAAAATCAAATTGAGGACACTCTATAAAATAAGTGGACTGAACTCCTTAAAAATGT
CAATGTCATGAAAGACAAAGAAAGGCTAAAGAATTCCATGAGGTCAAAGAACTATGACAACTAAACACAATTCTGGAT
GGAATATCAAATTAAAAAATAACAGATAAATAATATTATTGGGAAAGTTGAATAAATTTGAATATGGACTGTTTATTA
GTTATTAGTATTATAATAGTGTTAATTTTCCTAATTTTGTTAAGACTAGTGTGCCTGTTCCATGAAAATAGAAAATGT
TCTTATTCTCTGAAAATGCATGCTAAAGTATTTAGGGGTGAATGCAACAATGTCTGCAGCTCATTCTTGAATCAGTTC
AAAGAAAAATGAGTTACATTTATATATATATGTATGTATGTAAACAGACATAGATAAAAGTATAGATGTGTGTGTGTC
TTTAGAAAGGGGAGGATTTTTTTTTTTTTTTTGCTGTGTGTTACTGAAGTGCCTATGTCTGCGTGTTCACACTATCAT
ATTTTGTATGCCCTGGACTTTATAATTTCTACCTTCAAAATTAGATCTACTGTTGGTAATTAATTCAATATATACTGG
TTTTTTAACTACTATTCTCATTTCCTAGCAGTAATCTTCCTGAAAAGTCACAGAAATGATTACATTCCTTGTTCTTCA
TAATAATCACTGTTTAATTAAAATAAGAATATTTTAGAAAAGATCTGCGGCATAGTGGTTAAGACCCCAGTATTTGAT
GCTAAACAGATCTGATTTGGATAACAGAAGGTGGCACTTTGCTGTTTAAGCTGGGGACCAGACACTGTGGGTATAAAT
AGTAATTCCAAACACAGCTCCACAGAGCAGCACCCTTATGACAAGGTTTTCATATGTCTATAGTTAAGCCAGAAAATT
AAGAATAATGCCATAAATATTTATAAAGCTGAACATATCCAAGTTAAAGACCTTTATCCTGAAATTGTATCTTTTAGA
TTATTTTCTAAAGACTAATACCATTTAATGTTTAAATGTTCTTTGGAAATGATGGTGAGAATACGTGATAATGGGTCA
TTGGTTTTAATATTTTATTTAGCCAAGTGGAAAATTGGCAACCTGGTGTCGGTCCTCCCATTTGTATTTTACTGGTGC
ATGAAATCCAAAAGTCTAGTAACCATTGGGACAGACAACTCTACTGCATAAGTTTGTATGTTTGTATATCTGTATCAC
AAAGCCCAGACACTCGAACTATATAAACTTGTCGCACTAAAGACAGCAAATATGTCTGGTAATTGCATATTCTTCATG
TGTGCACTGGAATTTCTTATTATATAAGAAAATAAATGTGTTTCTAAACCACCATGAATTGGGTACTGCTGATAGTAC
TATTCTTCCTGGCACATGGAAATATCCCACTGAGGTTGTCAATCAATATTGATTTAATCAGTGTGGCAGCAAAGGCAC
TAGAGGAGGAAAATACTCTAGACAATCAAGTATAGATGGGAAAATTGCATCACCAAGAAATTCTTAAAATCACAAGTG
GATAATTTGCAATATCAATTGATTTTACTCAACTGCTTGAGTCCTTTATCATTCCGTTAGAACACTCCTCATATTTGC
AGATAACTCAGCCCCTCACCTCAGCAACAAGCAAGAGGACATCATTATAAACTTCCTCAATTTCTTTCCTATCCAGTG
AAAAATATCTAAGTCCCTTTTCCCTTCCCTTTTCCTTCCCTTCCCTTCCTTTTTCTTTCCTTCCATTCTCTTCCTTTT
TCCTTCCTTTTTCTTTCCTTCCATTCTCTTCCTTTTTCCTTCCTTTTTCTTACTTTACCTCCCCCTCCTACCTTTCCC
TTTCTCCCTTCCTCCCTCTCTCCCTTCCTTCCTCTCTCCCTCTCCTTCCTTCCTTCCTTCAATCCATACTTTTATATC
TTTTCAGAGATCAATTTTTCCCATTCATTTCTACATTCTCAGATACCTTGCTCCATGACTTGTTCTACTTCTCTTCTA
TCTTTATTCTTTCCCTCTTGACTGATCCTTTTTGTCTGCCTTTGCATTTTCAAAATCAGTTTTTCTAATCTTAAAAAA
TAACTTTGCTCAACCCTGATGCATCTTTATTCACTGTGTCATTATCTCATTATTTTTAAAGGTTAGTTTCTAAAGCCA
GGGCTCTATACTCACTACCTTCACTCTTGGTTAAAATTAAAAGAAATATAGGTCCCAGTGATATTTATGGGAAAATTT
GACTGTGAATAATCTTGTTGATATAAACACTTTGGGTTTTCAAAATGTTATGCTAGCTGTTAACTTTCCCAGTAACAT
TATGAGGGAAGGGCTGGAAAATACAAAAGGTAGACATAGGAACCGAGAACACCACCCCTATGGAGCCAAGCTATACAT
ACTCTGTCTCTGGTTATCATAAATACATCTCCTATATTCAATGCTTGCTTAGAAAGGCATGATGTCCACGGAGCAATT
CAATTGTTCACTGTGATGGGTTTTTTTGTTTTCTCCTCATGGAATGCAACTTTTATTTAT
SEQ ID NO: 17 >NM_006920.6 SCN1A [organism-Homo sapiens] [GeneID = 6323]
[transcript = 2]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGAACAGCATAAGAATTATTTCTGAGTGGAGGTGAGGCTTGTCCAAATGTCTTTGCTATCATGG
ATTTCCTGACTCCTACCTGTTTGAGGTTTGGGCAATTATGAATAAGGCTGCTGTATACATCCGTGTGCAGGATTTTGT
GTGGACATAAGTTTTCAACTCCTTTGGTTAAATCCTAAGGAATTTCATATGCAGAATAAATGGTAATTAAAATGTGCA
GGATGACAAGATGGAGCAAACAGTGCTTGTACCACCAGGACCTGACAGCTTCAACTTCTTCACCAGAGAATCTCTTGC
GGCTATTGAAAGACGCATTGCAGAAGAAAAGGCAAAGAATCCCAAACCAGACAAAAAAGATGACGACGAAAATGGCCC
AAAGCCAAATAGTGACTTGGAAGCTGGAAAGAACCTTCCATTTATTTATGGAGACATTCCTCCAGAGATGGTGTCAGA
GCCCCTGGAGGACCTGGACCCCTACTATATCAATAAGAAAACTTTTATAGTATTGAATAAAGGGAAGGCCATCTTCCG
GTTCAGTGCCACCTCTGCCCTGTACATTTTAACTCCCTTCAATCCTCTTAGGAAAATAGCTATTAAGATTTTGGTACA
TTCATTATTCAGCATGCTAATTATGTGCACTATTTTGACAAACTGTGTGTTTATGACAATGAGTAACCCTCCTGATTG
GACAAAGAATGTAGAATACACCTTCACAGGAATATATACTTTTGAATCACTTATAAAAATTATTGCAAGGGGATTCTG
TTTAGAAGATTTTACTTTCCTTCGGGATCCATGGAACTGGCTCGATTTCACTGTCATTACATTTGCGTACGTCACAGA
GTTTGTGGACCTGGGCAATGTCTCGGCATTGAGAACATTCAGAGTTCTCCGAGCATTGAAGACGATTTCAGTCATTCC
AGGCCTGAAAACCATTGTGGGAGCCCTGATCCAGTCTGTGAAGAAGCTCTCAGATGTAATGATCCTGACTGTGTTCTG
TCTGAGCGTATTTGCTCTAATTGGGCTGCAGCTGTTCATGGGCAACCTGAGGAATAAATGTATACAATGGCCTCCCAC
CAATGCTTCCTTGGAGGAACATAGTATAGAAAAGAATATAACTGTGAATTATAATGGTACACTTATAAATGAAACTGT
CTTTGAGTTTGACTGGAAGTCATATATTCAAGATTCAAGATATCATTATTTCCTGGAGGGTTTTTTAGATGCACTACT
ATGTGGAAATAGCTCTGATGCAGGCCAATGTCCAGAGGGATATATGTGTGTGAAAGCTGGTAGAAATCCCAATTATGG
CTACACAAGCTTTGATACCTTCAGTTGGGCTTTTTTGTCCTTGTTTCGACTAATGACTCAGGACTTCTGGGAAAATCT
TTATCAACTGACATTACGTGCTGCTGGGAAAACGTACATGATATTTTTTGTATTGGTCATTTTCTTGGGCTCATTCTA
CCTAATAAATTTGATCCTGGCTGTGGTGGCCATGGCCTACGAGGAACAGAATCAGGCCACCTTGGAAGAAGCAGAACA
GAAAGAGGCCGAATTTCAGCAGATGATTGAACAGCTTAAAAAGCAACAGGAGGCAGCTCAGCAGGCAGCAACGGCAAC
TGCCTCAGAACATTCCAGAGAGCCCAGTGCAGCAGGCAGGCTCTCAGACAGCTCATCTGAAGCCTCTAAGTTGAGTTC
CAAGAGTGCTAAGGAAAGAAGAAATCGGAGGAAGAAAAGAAAACAGAAAGAGCAGTCTGGTGGGGAAGAGAAAGATGA
GGATGAATTCCAAAAATCTGAATCTGAGGACAGCATCAGGAGGAAAGGTTTTCGCTTCTCCATTGAAGGGAACCGATT
GACATATGAAAAGAGGTACTCCTCCCCACACCAGTCTTTGTTGAGCATCCGTGGCTCCCTATTTTCACCAAGGCGAAA
TAGCAGAACAAGCCTTTTCAGCTTTAGAGGGCGAGCAAAGGATGTGGGATCTGAGAACGACTTCGCAGATGATGAGCA
CAGCACCTTTGAGGATAACGAGAGCCGTAGAGATTCCTTGTTTGTGCCCCGACGACACGGAGAGAGACGCAACAGCAA
CCTGAGTCAGACCAGTAGGTCATCCCGGATGCTGGCAGTGTTTCCAGCGAATGGGAAGATGCACAGCACTGTGGATTG
CAATGGTGTGGTTTCCTTGGTTGGTGGACCTTCAGTTCCTACATCGCCTGTTGGACAGCTTCTGCCAGAGGGAACAAC
CACTGAAACTGAAATGAGAAAGAGAAGGTCAAGTTCTTTCCACGTTTCCATGGACTTTCTAGAAGATCCTTCCCAAAG
GCAACGAGCAATGAGTATAGCCAGCATTCTAACAAATACAGTAGAAGAACTTGAAGAATCCAGGCAGAAATGCCCACC
CTGTTGGTATAAATTTTCCAACATATTCTTAATCTGGGACTGTTCTCCATATTGGTTAAAAGTGAAACATGTTGTCAA
CCTGGTTGTGATGGACCCATTTGTTGACCTGGCCATCACCATCTGTATTGTCTTAAATACTCTTTTCATGGCCATGGA
GCACTATCCAATGACGGACCATTTCAATAATGTGCTTACAGTAGGAAACTTGGTTTTCACTGGGATCTTTACAGCAGA
AATGTTTCTGAAAATTATTGCCATGGATCCTTACTATTATTTCCAAGAAGGCTGGAATATCTTTGACGGTTTTATTGT
GACGCTTAGCCTGGTAGAACTTGGACTCGCCAATGTGGAAGGATTATCTGTTCTCCGTTCATTTCGATTGCTGCGAGT
TTTCAAGTTGGCAAAATCTTGGCCAACGTTAAATATGCTAATAAAGATCATCGGCAATTCCGTGGGGGCTCTGGGAAA
TTTAACCCTCGTCTTGGCCATCATCGTCTTCATTTTTGCCGTGGTCGGCATGCAGCTCTTTGGTAAAAGCTACAAAGA
TTGTGTCTGCAAGATCGCCAGTGATTGTCAACTCCCACGCTGGCACATGAATGACTTCTTCCACTCCTTCCTGATTGT
GTTCCGCGTGCTGTGTGGGGAGTGGATAGAGACCATGTGGGACTGTATGGAGGTTGCTGGTCAAGCCATGTGCCTTAC
TGTCTTCATGATGGTCATGGTGATTGGAAACCTAGTGGTCCTGAATCTCTTTCTGGCCTTGCTTCTGAGCTCATTTAG
TGCAGACAACCTTGCAGCCACTGATGATGATAATGAAATGAATAATCTCCAAATTGCTGTGGATAGGATGCACAAAGG
AGTAGCTTATGTGAAAAGAAAAATATATGAATTTATTCAACAGTCCTTCATTAGGAAACAAAAGATTTTAGATGAAAT
TAAACCACTTGATGATCTAAACAACAAGAAAGACAGTTGTATGTCCAATCATACAGCAGAAATTGGGAAAGATCTTGA
CTATCTTAAAGATGTAAATGGAACTACAAGTGGTATAGGAACTGGCAGCAGTGTTGAAAAATACATTATTGATGAAAG
TGATTACATGTCATTCATAAACAACCCCAGTCTTACTGTGACTGTACCAATTGCTGTAGGAGAATCTGACTTTGAAAA
TTTAAACACGGAAGACTTTAGTAGTGAATCGGATCTGGAAGAAAGCAAAGAGAAACTGAATGAAAGCAGTAGCTCATC
AGAAGGTAGCACTGTGGACATCGGCGCACCTGTAGAAGAACAGCCCGTAGTGGAACCTGAAGAAACTCTTGAACCAGA
AGCTTGTTTCACTGAAGGCTGTGTACAAAGATTCAAGTGTTGTCAAATCAATGTGGAAGAAGGCAGAGGAAAACAATG
GTGGAACCTGAGAAGGACGTGTTTCCGAATAGTTGAACATAACTGGTTTGAGACCTTCATTGTTTTCATGATTCTCCT
TAGTAGTGGTGCTCTGGCATTTGAAGATATATATATTGATCAGCGAAAGACGATTAAGACGATGTTGGAATATGCTGA
CAAGGTTTTCACTTACATTTTCATTCTGGAAATGCTTCTAAAATGGGTGGCATATGGCTATCAAACATATTTCACCAA
TGCCTGGTGTTGGCTGGACTTCTTAATTGTTGATGTTTCATTGGTCAGTTTAACAGCAAATGCCTTGGGTTACTCAGA
ACTTGGAGCCATCAAATCTCTCAGGACACTAAGAGCTCTGAGACCTCTAAGAGCCTTATCTCGATTTGAAGGGATGAG
GGTGGTTGTGAATGCCCTTTTAGGAGCAATTCCATCCATCATGAATGTGCTTCTGGTTTGTCTTATATTCTGGCTAAT
TTTCAGCATCATGGGCGTAAATTTGTTTGCTGGCAAATTCTACCACTGTATTAACACCACAACTGGTGACAGGTTTGA
CATCGAAGACGTGAATAATCATACTGATTGCCTAAAACTAATAGAAAGAAATGAGACTGCTCGATGGAAAAATGTGAA
AGTAAACTTTGATAATGTAGGATTTGGGTATCTCTCTTTGCTTCAAGTTGCCACATTCAAAGGATGGATGGATATAAT
GTATGCAGCAGTTGATTCCAGAAATGTGGAACTCCAGCCTAAGTATGAAGAAAGTCTGTACATGTATCTTTACTTTGT
TATTTTCATCATCTTTGGGTCCTTCTTCACCTTGAACCTGTTTATTGGTGTCATCATAGATAATTTCAACCAGCAGAA
AAAGAAGTTTGGAGGTCAAGACATCTTTATGACAGAAGAACAGAAGAAATACTATAATGCAATGAAAAAATTAGGATC
GAAAAAACCGCAAAAGCCTATACCTCGACCAGGAAACAAATTTCAAGGAATGGTCTTTGACTTCGTAACCAGACAAGT
TTTTGACATAAGCATCATGATTCTCATCTGTCTTAACATGGTCACAATGATGGTGGAAACAGATGACCAGAGTGAATA
TGTGACTACCATTTTGTCACGCATCAATCTGGTGTTCATTGTGCTATTTACTGGAGAGTGTGTACTGAAACTCATCTC
TCTACGCCATTATTATTTTACCATTGGATGGAATATTTTTGATTTTGTGGTTGTCATTCTCTCCATTGTAGGTATGTT
TCTTGCCGAGCTGATAGAAAAGTATTTCGTGTCCCCTACCCTGTTCCGAGTGATCCGTCTTGCTAGGATTGGCCGAAT
CCTACGTCTGATCAAAGGAGCAAAGGGGATCCGCACGCTGCTCTTTGCTTTGATGATGTCCCTTCCTGCGTTGTTTAA
CATCGGCCTCCTACTCTTCCTAGTCATGTTCATCTACGCCATCTTTGGGATGTCCAACTTTGCCTATGTTAAGAGGGA
AGTTGGGATCGATGACATGTTCAACTTTGAGACCTTTGGCAACAGCATGATCTGCCTATTCCAAATTACAACCTCTGC
TGGCTGGGATGGATTGCTAGCACCCATTCTCAACAGTAAGCCACCCGACTGTGACCCTAATAAAGTTAACCCTGGAAG
CTCAGTTAAGGGAGACTGTGGGAACCCATCTGTTGGAATTTTCTTTTTTGTCAGTTACATCATCATATCCTTCCTGGT
TGTGGTGAACATGTACATCGCGGTCATCCTGGAGAACTTCAGTGTTGCTACTGAAGAAAGTGCAGAGCCTCTGAGTGA
GGATGACTTTGAGATGTTCTATGAGGTTTGGGAGAAGTTTGATCCCGATGCAACTCAGTTCATGGAATTTGAAAAATT
ATCTCAGTTTGCAGCTGCGCTTGAACCGCCTCTCAATCTGCCACAACCAAACAAACTCCAGCTCATTGCCATGGATTT
GCCCATGGTGAGTGGTGACCGGATCCACTGTCTTGATATCTTATTTGCTTTTACAAAGCGGGTTCTAGGAGAGAGTGG
AGAGATGGATGCTCTACGAATACAGATGGAAGAGCGATTCATGGCTTCCAATCCTTCCAAGGTCTCCTATCAGCCAAT
CACTACTACTTTAAAACGAAAACAAGAGGAAGTATCTGCTGTCATTATTCAGCGTGCTTACAGACGCCACCTTTTAAA
GCGAACTGTAAAACAAGCTTCCTTTACGTACAATAAAAACAAAATCAAAGGTGGGGCTAATCTTCTTATAAAAGAAGA
CATGATAATTGACAGAATAAATGAAAACTCTATTACAGAAAAAACTGATCTGACCATGTCCACTGCAGCTTGTCCACC
TTCCTATGACCGGGTGACAAAGCCAATTGTGGAAAAACATGAGCAAGAAGGCAAAGATGAAAAAGCCAAAGGGAAATA
AATGAAAATAAATAAAAATAATTGGGTGACAAATTGTTTACAGCCTGTGAAGGTGATGTATTTTTATCAACAGGACTC
CTTTAGGAGGTCAATGCCAAACTGACTGTTTTTACACAAATCTCCTTAAGGTCAGTGCCTACAATAAGACAGTGACCC
CTTGTCAGCAAACTGTGACTCTGTGTAAAGGGGAGATGACCTTGACAGGAGGTTACTGTTCTCACTACCAGCTGACAC
TGCTGAAGATAAGATGCACAATGGCTAGTCAGACTGTAGGGACCAGTTTCAAGGGGTGCAAACCTGTGATTTTGGGGT
TGTTTAACATGAAACACTTTAGTGTAGTAATTGTATCCACTGTTTGCATTTCAACTGCCACATTTGTCACATTTTTAT
GGAATCTGTTAGTGGATTCATCTTTTTGTTAATCCATGTGTTTATTATATGTGACTATTTTTGTAAACGAAGTTTCTG
TTGAGAAATAGGCTAAGGACCTCTATAACAGGTATGCCACCTGGGGGGTATGGCAACCACATGGCCCTCCCAGCTACA
CAAAGTCGTGGTTTGCATGAGGGCATGCTGCACTTAGAGATCATGCATGAGAAAAAGTCACAAGAAAAACAAATTCTT
AAATTTCACCATATTTCTGGGAGGGGTAATTGGGTGATAAGTGGAGGTGCTTTGTTGATCTTGTTTTGCGAAATCCAG
CCCCTAGACCAAGTAGATTATTTGTGGGTAGGCCAGTAAATCTTAGCAGGTGCAAACTTCATTCAAATGTTTGGAGTC
ATAAATGTTATGTTTCTTTTTGTTGTATTAAAAAAAAAACCTGAATAGTGAATATTGCCCCTCACCCTCCACCGCCAG
AAGACTGAATTGACCAAAATTACTCTTTATAAATTTCTGCTTTTTCCTGCACTTTGTTTAGCCATCTTCGGCTCTCAG
CAAGGTTGACACTGTATATGTTAATGAAATGCTATTTATTATGTAAATAGTCATTTTACCCTGTGGTGCACGTTTGAG
CAAACAAATAATGACCTAAGCACAGTATTTATTGCATCAAATATGTACCACAAGAAATGTAGAGTGCAAGCTTTACAC
AGGTAATAAAATGTATTCTGTACCATTTATAGATAGTTTGGATGCTATCAATGCATGTTTATATTACCATGCTGCTGT
ATCTGGTTTCTCTCACTGCTCAGAATCTCATTTATGAGAAACCATATGTCAGTGGTAAAGTCAAGGAAATTGTTCAAC
AGATCTCATTTATTTAAGTCATTAAGCAATAGTTTGCAGCACTTTAACAGCTTTTTGGTTATTTTTACATTTTAAGTG
GATAACATATGGTATATAGCCAGACTGTACAGACATGTTTAAAAAAACACACTGCTTAACCTATTAAATATGTGTTTA
GAATTTTATAAGCAAATATAAATACTGTAAAAAGTCACTTTATTTTATTTTTCAGCATTATGTACATAAATATGAAGA
GGAAATTATCTTCAGGTTGATATCACAATCACTTTTCTTACTTTCTGTCCATAGTACTTTTTCATGAAAGAAATTTGC
TAAATAAGACATGAAAACAAGACTGGGTAGTTGTAGATTTCTGCTTTTTAAATTACATTTGCTAATTTTAGATTATTT
CACAATTTTAAGGAGCAAAATAGGTTCACGATTCATATCCAAATTATGCTTTGCAATTGGAAAAGGGTTTAAAATTTT
ATTTATATTTCTGGTAGTACCTGCACTAACTGAATTGAAGGTAGTGCTTATGTTATTTTTGTTCTTTTTTTCTGACTT
CGGTTTATGTTTTCATTTCTTTGGAGTAATGCTGCTCTAGATTGTTCTAAATAGAATGTGGGCTTCATAATTTTTTTT
TCCACAAAAACAGAGTAGTCAACTTATATAGTCAATTACATCAGGACATTTTGTGTTTCTTACAGAAGCAAACCATAG
GCTCCTCTTTTCCTTAAAACTACTTAGATAAACTGTATTCGTGAACTGCATGCTGGAAAATGCTACTATTATGCTAAA
TAATGCTAACCAACATTTAAAATGTGCAAAACTAATAAAGATTACATTTTTTATTTTATTGTTTGCCCAGTCACTTTT
TGTTAACAGAATATTCTAATGATATGGAGATTTTTTACATTACAAATTGGGGGAGAAGGGGAGCGCGCGCGCACACAC
ACACACACACACACACACACACACACACACACAGAGGCATACCCACGTTGACAACAAAACCTAGGGTAGATATGTCAC
TGGAGGTAGGGGGTAATGACCTCCCAGAATTACAAGCAGCAGGTGTGTTCTCTGTTAGGAGGAAGAACTGGTGTCAGA
GGATAGCTAGTGATTCTAGGAGGAAGAGAAGTATGGAAGCCAGAGTGATGGTGGATGACCCCTTGAGCTATGAAAAGA
AACCCTTAAATCATCATTTAAAAATTTAGAATTGCCATGTGTGTAGGATACTGTGTTTGCTCCTCCAGAGCCACTCTC
TCTGCTTCTGCATCATTCTGTGTGTCCCAGAAGGGTGACTTCTACACATTGCAAAAATGGGCTCTCCTACCTTTGAGC
TCCCAATTGGTTTGGCCAATGAGAAGCACCAGTGGGAAAGCACCAGAGAGAGAAGATTGACATAGGAATATTTCTTCT
CCAATTCCTTCTTTGCTGGGTTGGCACTGGACTCATTCCTCCCCGAAAAGTCATACTCCAATCAGACTGCCCCTCATA
CAACTGAAGCTACTTTCTCTGGGGTCAGGTAATCACTCCTCCCCTTGCTCCTTCAGGTCTGCTGCTGCATTGAGAGTG
CTTTTGTATTCCTTGTAGCTTTCTCCTAACATTGCTGACACTTTTGTAAATGTCCCCTTCATGAAATTCTTCTATATG
CCTCATTTCAGCATGCCATCTGTCTCCTGCCTGGCTGACACAAGGTGATTCAACAGCTCATGAAAGTCAGCAGGAAGC
AAAGATGTGCCTTGCTTCAGCTTGGGGTCTTAATCTTGCTAACTTTTGCAGATAAAGAAAAACAGTAACTGGGGGAAC
CACAGTGAAGTCCAGTGCAGAATTCACAGATATCATGGAAAGGTTACTCGGGTGGTCCAGATAGTAAAATTAACAGTC
TAAATTAATCTATCTAAATTTCTGAGGAACGAGAAGCCTTCCCTTGTCATCAGGTGAAGCCAGAAGAGGGAATATAGC
CTCAACCAGAAAAGGGACAGTAATTAAAAGGCTTTTCCCATCCTTGTACAATGGACTGACTTTGCCTCTTCATAACAT
CACAATCCTAAAGCAACACAACAATTAATTCTGATATATTAGTAGCTGAAAAAAATTCCCATTTCCAACTAAGGTAGG
TCAGAATTATAGGATAAACCCTGCAGACTTTTTATACTACCCATCCACGCCATTACTCACTGTTACCTTTCCAAATAC
AAAGAGAAGAACTGGTAAAACATAATCATATAAATCTCCATATTCATTTTGAAATATTTGGCATGATATTTTCTGTGC
TAAAAAGTAATTATTCTTCAAAGAATGATGAGGTCATGTCAGTAAGACACAGGAACCAACTAGAAGGGGCTTCCCACT
GGCCAAATCTGGGGCAAGTTGAGCATCAAAATAAATGATAGTAAAAGATTATAATTCATTGAATAAGAATCAGCAAAT
ACATACTGATGTAAGTAAATAAGGAAAAGTACAAATCTGTTTCTTGCAGTTGAATGTTAATTAACAATTGTAGAAGAA
ATAACGGAGTTAGAAAAATCACTATTTGGCAATCACCCTAATGACAATTGATTCATACAAGAATCATCAATGAGTATT
AAAACTCATGGGTGAAAGTTTGATGAGGAATAGGGTATTTATAGCATCTTAAAGTATCTCTTCTCTATTAAGTAGAAA
ATTTAAACAGAAGAAAGTATACTTTGGAGAAATACAGCAGACAATACCTTCAAAGATATCATCAATTATGAGACCAAC
TGATACTATGTGCCTCCTGATAAGATATACTGAAAGGGCCACATTACTTCTTGGTACACAGTCAAAATTTTAAAACCA
GAATCTAACTACAAGGAAAATCAAATTGAGGACACTCTATAAAATAAGTGGACTGAACTCCTTAAAAATGTCAATGTC
ATGAAAGACAAAGAAAGGCTAAAGAATTCCATGAGGTCAAAGAACTATGACAACTAAACACAATTCTGGATGGAATAT
CAAATTAAAAAATAACAGATAAATAATATTATTGGGAAAGTTGAATAAATTTGAATATGGACTGTTTATTAGTTATTA
GTATTATAATAGTGTTAATTTTCCTAATTTTGTTAAGACTAGTGTGCCTGTTCCATGAAAATAGAAAATGTTCTTATT
CTCTGAAAATGCATGCTAAAGTATTTAGGGGTGAATGCAACAATGTCTGCAGCTCATTCTTGAATCAGTTCAAAGAAA
AATGAGTTACATTTATATATATATGTATGTATGTAAACAGACATAGATAAAAGTATAGATGTGTGTGTGTCTTTAGAA
AGGGGAGGATTTTTTTTTTTTTTTTGCTGTGTGTTACTGAAGTGCCTATGTCTGCGTGTTCACACTATCATATTTTGT
ATGCCCTGGACTTTATAATTTCTACCTTCAAAATTAGATCTACTGTTGGTAATTAATTCAATATATACTGGTTTTTTA
ACTACTATTCTCATTTCCTAGCAGTAATCTTCCTGAAAAGTCACAGAAATGATTACATTCCTTGTTCTTCATAATAAT
CACTGTTTAATTAAAATAAGAATATTTTAGAAAAGATCTGCGGCATAGTGGTTAAGACCCCAGTATTTGATGCTAAAC
AGATCTGATTTGGATAACAGAAGGTGGCACTTTGCTGTTTAAGCTGGGGACCAGACACTGTGGGTATAAATAGTAATT
CCAAACACAGCTCCACAGAGCAGCACCCTTATGACAAGGTTTTCATATGTCTATAGTTAAGCCAGAAAATTAAGAATA
ATGCCATAAATATTTATAAAGCTGAACATATCCAAGTTAAAGACCTTTATCCTGAAATTGTATCTTTTAGATTATTTT
CTAAAGACTAATACCATTTAATGTTTAAATGTTCTTTGGAAATGATGGTGAGAATACGTGATAATGGGTCATTGGTTT
TAATATTTTATTTAGCCAAGTGGAAAATTGGCAACCTGGTGTCGGTCCTCCCATTTGTATTTTACTGGTGCATGAAAT
CCAAAAGTCTAGTAACCATTGGGACAGACAACTCTACTGCATAAGTTTGTATGTTTGTATATCTGTATCACAAAGCCC
AGACACTCGAACTATATAAACTTGTCGCACTAAAGACAGCAAATATGTCTGGTAATTGCATATTCTTCATGTGTGCAC
TGGAATTTCTTATTATATAAGAAAATAAATGTGTTTCTAAACCACCATGAATTGGGTACTGCTGATAGTACTATTCTT
CCTGGCACATGGAAATATCCCACTGAGGTTGTCAATCAATATTGATTTAATCAGTGTGGCAGCAAAGGCACTAGAGGA
GGAAAATACTCTAGACAATCAAGTATAGATGGGAAAATTGCATCACCAAGAAATTCTTAAAATCACAAGTGGATAATT
TGCAATATCAATTGATTTTACTCAACTGCTTGAGTCCTTTATCATTCCGTTAGAACACTCCTCATATTTGCAGATAAC
TCAGCCCCTCACCTCAGCAACAAGCAAGAGGACATCATTATAAACTTCCTCAATTTCTTTCCTATCCAGTGAAAAATA
TCTAAGTCCCTTTTCCCTTCCCTTTTCCTTCCCTTCCCTTCCTTTTTCTTTCCTTCCATTCTCTTCCTTTTTCCTTCC
TTTTTCTTTCCTTCCATTCTCTTCCTTTTTCCTTCCTTTTTCTTACTTTACCTCCCCCTCCTACCTTTCCCTTTCTCC
CTTCCTCCCTCTCTCCCTTCCTTCCTCTCTCCCTCTCCTTCCTTCCTTCCTTCAATCCATACTTTTATATCTTTTCAG
AGATCAATTTTTCCCATTCATTTCTACATTCTCAGATACCTTGCTCCATGACTTGTTCTACTTCTCTTCTATCTTTAT
TCTTTCCCTCTTGACTGATCCTTTTTGTCTGCCTTTGCATTTTCAAAATCAGTTTTTCTAATCTTAAAAAATAACTTT
GCTCAACCCTGATGCATCTTTATTCACTGTGTCATTATCTCATTATTTTTAAAGGTTAGTTTCTAAAGCCAGGGCTCT
ATACTCACTACCTTCACTCTTGGTTAAAATTAAAAGAAATATAGGTCCCAGTGATATTTATGGGAAAATTTGACTGTG
AATAATCTTGTTGATATAAACACTTTGGGTTTTCAAAATGTTATGCTAGCTGTTAACTTTCCCAGTAACATTATGAGG
GAAGGGCTGGAAAATACAAAAGGTAGACATAGGAACCGAGAACACCACCCCTATGGAGCCAAGCTATACATACTCTGT
CTCTGGTTATCATAAATACATCTCCTATATTCAATGCTTGCTTAGAAAGGCATGATGTCCACGGAGCAATTCAATTGT
TCACTGTGATGGGTTTTTTTGTTTTCTCCTCATGGAATGCAACTTTTATTTAT
SEQ ID NO: 18 >NM_001165963.4 SCN1A [organism = Homo sapiens] [GeneID = 6323]
[transcript = 1]
ACCATAGAGTGAGGCGAGGATGAAGCCGAGAGGATACTGCAGAGGTCTCTGGTGCATGTGTGTATGTGTGCGTTTGTG
TGTGTTTGTGTGTCTGTGTGTTCTGCCCCAGTGAGACTGCAGCCCTTGTAAATACTTTGACACCTTTTGCAAGAAGGA
ATCTGAACAATTGCAACTGAAGGCACATTGTTATCATCTCGTCTTTGGGTGATGCTGTTCCTCACTGCAGATGGATAA
TTTTCCTTTTAATCAGAACAGCATAAGAATTATTTCTGAGTGGAGGTGAGGCTTGTCCAAATGTCTTTGCTATCATGG
ATTTCCTGACTCCTACCTGTTTGAGGTTTGGGCAATTATGAATAAGGCTGCTGTATACATCCGTGTGCAGGATTTTGT
GTGGACATAAGTTTTCAACTCCTTTGGTTAAATCCTAAGGAATTTCATATGCAGAATAAATGGTAATTAAAATGTGCA
GGATGACAAGATGGAGCAAACAGTGCTTGTACCACCAGGACCTGACAGCTTCAACTTCTTCACCAGAGAATCTCTTGC
GGCTATTGAAAGACGCATTGCAGAAGAAAAGGCAAAGAATCCCAAACCAGACAAAAAAGATGACGACGAAAATGGCCC
AAAGCCAAATAGTGACTTGGAAGCTGGAAAGAACCTTCCATTTATTTATGGAGACATTCCTCCAGAGATGGTGTCAGA
GCCCCTGGAGGACCTGGACCCCTACTATATCAATAAGAAAACTTTTATAGTATTGAATAAAGGGAAGGCCATCTTCCG
GTTCAGTGCCACCTCTGCCCTGTACATTTTAACTCCCTTCAATCCTCTTAGGAAAATAGCTATTAAGATTTTGGTACA
TTCATTATTCAGCATGCTAATTATGTGCACTATTTTGACAAACTGTGTGTTTATGACAATGAGTAACCCTCCTGATTG
GACAAAGAATGTAGAATACACCTTCACAGGAATATATACTTTTGAATCACTTATAAAAATTATTGCAAGGGGATTCTG
TTTAGAAGATTTTACTTTCCTTCGGGATCCATGGAACTGGCTCGATTTCACTGTCATTACATTTGCGTACGTCACAGA
GTTTGTGGACCTGGGCAATGTCTCGGCATTGAGAACATTCAGAGTTCTCCGAGCATTGAAGACGATTTCAGTCATTCC
AGGCCTGAAAACCATTGTGGGAGCCCTGATCCAGTCTGTGAAGAAGCTCTCAGATGTAATGATCCTGACTGTGTTCTG
TCTGAGCGTATTTGCTCTAATTGGGCTGCAGCTGTTCATGGGCAACCTGAGGAATAAATGTATACAATGGCCTCCCAC
CAATGCTTCCTTGGAGGAACATAGTATAGAAAAGAATATAACTGTGAATTATAATGGTACACTTATAAATGAAACTGT
CTTTGAGTTTGACTGGAAGTCATATATTCAAGATTCAAGATATCATTATTTCCTGGAGGGTTTTTTAGATGCACTACT
ATGTGGAAATAGCTCTGATGCAGGCCAATGTCCAGAGGGATATATGTGTGTGAAAGCTGGTAGAAATCCCAATTATGG
CTACACAAGCTTTGATACCTTCAGTTGGGCTTTTTTGTCCTTGTTTCGACTAATGACTCAGGACTTCTGGGAAAATCT
TTATCAACTGACATTACGTGCTGCTGGGAAAACGTACATGATATTTTTTGTATTGGTCATTTTCTTGGGCTCATTCTA
CCTAATAAATTTGATCCTGGCTGTGGTGGCCATGGCCTACGAGGAACAGAATCAGGCCACCTTGGAAGAAGCAGAACA
GAAAGAGGCCGAATTTCAGCAGATGATTGAACAGCTTAAAAAGCAACAGGAGGCAGCTCAGCAGGCAGCAACGGCAAC
TGCCTCAGAACATTCCAGAGAGCCCAGTGCAGCAGGCAGGCTCTCAGACAGCTCATCTGAAGCCTCTAAGTTGAGTTC
CAAGAGTGCTAAGGAAAGAAGAAATCGGAGGAAGAAAAGAAAACAGAAAGAGCAGTCTGGTGGGGAAGAGAAAGATGA
GGATGAATTCCAAAAATCTGAATCTGAGGACAGCATCAGGAGGAAAGGTTTTCGCTTCTCCATTGAAGGGAACCGATT
GACATATGAAAAGAGGTACTCCTCCCCACACCAGTCTTTGTTGAGCATCCGTGGCTCCCTATTTTCACCAAGGCGAAA
TAGCAGAACAAGCCTTTTCAGCTTTAGAGGGCGAGCAAAGGATGTGGGATCTGAGAACGACTTCGCAGATGATGAGCA
CAGCACCTTTGAGGATAACGAGAGCCGTAGAGATTCCTTGTTTGTGCCCCGACGACACGGAGAGAGACGCAACAGCAA
CCTGAGTCAGACCAGTAGGTCATCCCGGATGCTGGCAGTGTTTCCAGCGAATGGGAAGATGCACAGCACTGTGGATTG
CAATGGTGTGGTTTCCTTGGTTGGTGGACCTTCAGTTCCTACATCGCCTGTTGGACAGCTTCTGCCAGAGGTGATAAT
AGATAAGCCAGCTACTGATGACAATGGAACAACCACTGAAACTGAAATGAGAAAGAGAAGGTCAAGTTCTTTCCACGT
TTCCATGGACTTTCTAGAAGATCCTTCCCAAAGGCAACGAGCAATGAGTATAGCCAGCATTCTAACAAATACAGTAGA
AGAACTTGAAGAATCCAGGCAGAAATGCCCACCCTGTTGGTATAAATTTTCCAACATATTCTTAATCTGGGACTGTTC
TCCATATTGGTTAAAAGTGAAACATGTTGTCAACCTGGTTGTGATGGACCCATTTGTTGACCTGGCCATCACCATCTG
TATTGTCTTAAATACTCTTTTCATGGCCATGGAGCACTATCCAATGACGGACCATTTCAATAATGTGCTTACAGTAGG
AAACTTGGTTTTCACTGGGATCTTTACAGCAGAAATGTTTCTGAAAATTATTGCCATGGATCCTTACTATTATTTCCA
AGAAGGCTGGAATATCTTTGACGGTTTTATTGTGACGCTTAGCCTGGTAGAACTTGGACTCGCCAATGTGGAAGGATT
ATCTGTTCTCCGTTCATTTCGATTGCTGCGAGTTTTCAAGTTGGCAAAATCTTGGCCAACGTTAAATATGCTAATAAA
GATCATCGGCAATTCCGTGGGGGCTCTGGGAAATTTAACCCTCGTCTTGGCCATCATCGTCTTCATTTTTGCCGTGGT
CGGCATGCAGCTCTTTGGTAAAAGCTACAAAGATTGTGTCTGCAAGATCGCCAGTGATTGTCAACTCCCACGCTGGCA
CATGAATGACTTCTTCCACTCCTTCCTGATTGTGTTCCGCGTGCTGTGTGGGGAGTGGATAGAGACCATGTGGGACTG
TATGGAGGTTGCTGGTCAAGCCATGTGCCTTACTGTCTTCATGATGGTCATGGTGATTGGAAACCTAGTGGTCCTGAA
TCTCTTTCTGGCCTTGCTTCTGAGCTCATTTAGTGCAGACAACCTTGCAGCCACTGATGATGATAATGAAATGAATAA
TCTCCAAATTGCTGTGGATAGGATGCACAAAGGAGTAGCTTATGTGAAAAGAAAAATATATGAATTTATTCAACAGTC
CTTCATTAGGAAACAAAAGATTTTAGATGAAATTAAACCACTTGATGATCTAAACAACAAGAAAGACAGTTGTATGTC
CAATCATACAGCAGAAATTGGGAAAGATCTTGACTATCTTAAAGATGTAAATGGAACTACAAGTGGTATAGGAACTGG
CAGCAGTGTTGAAAAATACATTATTGATGAAAGTGATTACATGTCATTCATAAACAACCCCAGTCTTACTGTGACTGT
ACCAATTGCTGTAGGAGAATCTGACTTTGAAAATTTAAACACGGAAGACTTTAGTAGTGAATCGGATCTGGAAGAAAG
CAAAGAGAAACTGAATGAAAGCAGTAGCTCATCAGAAGGTAGCACTGTGGACATCGGCGCACCTGTAGAAGAACAGCC
CGTAGTGGAACCTGAAGAAACTCTTGAACCAGAAGCTTGTTTCACTGAAGGCTGTGTACAAAGATTCAAGTGTTGTCA
AATCAATGTGGAAGAAGGCAGAGGAAAACAATGGTGGAACCTGAGAAGGACGTGTTTCCGAATAGTTGAACATAACTG
GTTTGAGACCTTCATTGTTTTCATGATTCTCCTTAGTAGTGGTGCTCTGGCATTTGAAGATATATATATTGATCAGCG
AAAGACGATTAAGACGATGTTGGAATATGCTGACAAGGTTTTCACTTACATTTTCATTCTGGAAATGCTTCTAAAATG
GGTGGCATATGGCTATCAAACATATTTCACCAATGCCTGGTGTTGGCTGGACTTCTTAATTGTTGATGTTTCATTGGT
CAGTTTAACAGCAAATGCCTTGGGTTACTCAGAACTTGGAGCCATCAAATCTCTCAGGACACTAAGAGCTCTGAGACC
TCTAAGAGCCTTATCTCGATTTGAAGGGATGAGGGTGGTTGTGAATGCCCTTTTAGGAGCAATTCCATCCATCATGAA
TGTGCTTCTGGTTTGTCTTATATTCTGGCTAATTTTCAGCATCATGGGCGTAAATTTGTTTGCTGGCAAATTCTACCA
CTGTATTAACACCACAACTGGTGACAGGTTTGACATCGAAGACGTGAATAATCATACTGATTGCCTAAAACTAATAGA
AAGAAATGAGACTGCTCGATGGAAAAATGTGAAAGTAAACTTTGATAATGTAGGATTTGGGTATCTCTCTTTGCTTCA
AGTTGCCACATTCAAAGGATGGATGGATATAATGTATGCAGCAGTTGATTCCAGAAATGTGGAACTCCAGCCTAAGTA
TGAAGAAAGTCTGTACATGTATCTTTACTTTGTTATTTTCATCATCTTTGGGTCCTTCTTCACCTTGAACCTGTTTAT
TGGTGTCATCATAGATAATTTCAACCAGCAGAAAAAGAAGTTTGGAGGTCAAGACATCTTTATGACAGAAGAACAGAA
GAAATACTATAATGCAATGAAAAAATTAGGATCGAAAAAACCGCAAAAGCCTATACCTCGACCAGGAAACAAATTTCA
AGGAATGGTCTTTGACTTCGTAACCAGACAAGTTTTTGACATAAGCATCATGATTCTCATCTGTCTTAACATGGTCAC
AATGATGGTGGAAACAGATGACCAGAGTGAATATGTGACTACCATTTTGTCACGCATCAATCTGGTGTTCATTGTGCT
ATTTACTGGAGAGTGTGTACTGAAACTCATCTCTCTACGCCATTATTATTTTACCATTGGATGGAATATTTTTGATTT
TGTGGTTGTCATTCTCTCCATTGTAGGTATGTTTCTTGCCGAGCTGATAGAAAAGTATTTCGTGTCCCCTACCCTGTT
CCGAGTGATCCGTCTTGCTAGGATTGGCCGAATCCTACGTCTGATCAAAGGAGCAAAGGGGATCCGCACGCTGCTCTT
TGCTTTGATGATGTCCCTTCCTGCGTTGTTTAACATCGGCCTCCTACTCTTCCTAGTCATGTTCATCTACGCCATCTT
TGGGATGTCCAACTTTGCCTATGTTAAGAGGGAAGTTGGGATCGATGACATGTTCAACTTTGAGACCTTTGGCAACAG
CATGATCTGCCTATTCCAAATTACAACCTCTGCTGGCTGGGATGGATTGCTAGCACCCATTCTCAACAGTAAGCCACC
CGACTGTGACCCTAATAAAGTTAACCCTGGAAGCTCAGTTAAGGGAGACTGTGGGAACCCATCTGTTGGAATTTTCTT
TTTTGTCAGTTACATCATCATATCCTTCCTGGTTGTGGTGAACATGTACATCGCGGTCATCCTGGAGAACTTCAGTGT
TGCTACTGAAGAAAGTGCAGAGCCTCTGAGTGAGGATGACTTTGAGATGTTCTATGAGGTTTGGGAGAAGTTTGATCC
CGATGCAACTCAGTTCATGGAATTTGAAAAATTATCTCAGTTTGCAGCTGCGCTTGAACCGCCTCTCAATCTGCCACA
ACCAAACAAACTCCAGCTCATTGCCATGGATTTGCCCATGGTGAGTGGTGACCGGATCCACTGTCTTGATATCTTATT
TGCTTTTACAAAGCGGGTTCTAGGAGAGAGTGGAGAGATGGATGCTCTACGAATACAGATGGAAGAGCGATTCATGGC
TTCCAATCCTTCCAAGGTCTCCTATCAGCCAATCACTACTACTTTAAAACGAAAACAAGAGGAAGTATCTGCTGTCAT
TATTCAGCGTGCTTACAGACGCCACCTTTTAAAGCGAACTGTAAAACAAGCTTCCTTTACGTACAATAAAAACAAAAT
CAAAGGTGGGGCTAATCTTCTTATAAAAGAAGACATGATAATTGACAGAATAAATGAAAACTCTATTACAGAAAAAAC
TGATCTGACCATGTCCACTGCAGCTTGTCCACCTTCCTATGACCGGGTGACAAAGCCAATTGTGGAAAAACATGAGCA
AGAAGGCAAAGATGAAAAAGCCAAAGGGAAATAAATGAAAATAAATAAAAATAATTGGGTGACAAATTGTTTACAGCC
TGTGAAGGTGATGTATTTTTATCAACAGGACTCCTTTAGGAGGTCAATGCCAAACTGACTGTTTTTACACAAATCTCC
TTAAGGTCAGTGCCTACAATAAGACAGTGACCCCTTGTCAGCAAACTGTGACTCTGTGTAAAGGGGAGATGACCTTGA
CAGGAGGTTACTGTTCTCACTACCAGCTGACACTGCTGAAGATAAGATGCACAATGGCTAGTCAGACTGTAGGGACCA
GTTTCAAGGGGTGCAAACCTGTGATTTTGGGGTTGTTTAACATGAAACACTTTAGTGTAGTAATTGTATCCACTGTTT
GCATTTCAACTGCCACATTTGTCACATTTTTATGGAATCTGTTAGTGGATTCATCTTTTTGTTAATCCATGTGTTTAT
TATATGTGACTATTTTTGTAAACGAAGTTTCTGTTGAGAAATAGGCTAAGGACCTCTATAACAGGTATGCCACCTGGG
GGGTATGGCAACCACATGGCCCTCCCAGCTACACAAAGTCGTGGTTTGCATGAGGGCATGCTGCACTTAGAGATCATG
CATGAGAAAAAGTCACAAGAAAAACAAATTCTTAAATTTCACCATATTTCTGGGAGGGGTAATTGGGTGATAAGTGGA
GGTGCTTTGTTGATCTTGTTTTGCGAAATCCAGCCCCTAGACCAAGTAGATTATTTGTGGGTAGGCCAGTAAATCTTA
GCAGGTGCAAACTTCATTCAAATGTTTGGAGTCATAAATGTTATGTTTCTTTTTGTTGTATTAAAAAAAAAACCTGAA
TAGTGAATATTGCCCCTCACCCTCCACCGCCAGAAGACTGAATTGACCAAAATTACTCTTTATAAATTTCTGCTTTTT
CCTGCACTTTGTTTAGCCATCTTCGGCTCTCAGCAAGGTTGACACTGTATATGTTAATGAAATGCTATTTATTATGTA
AATAGTCATTTTACCCTGTGGTGCACGTTTGAGCAAACAAATAATGACCTAAGCACAGTATTTATTGCATCAAATATG
TACCACAAGAAATGTAGAGTGCAAGCTTTACACAGGTAATAAAATGTATTCTGTACCATTTATAGATAGTTTGGATGC
TATCAATGCATGTTTATATTACCATGCTGCTGTATCTGGTTTCTCTCACTGCTCAGAATCTCATTTATGAGAAACCAT
ATGTCAGTGGTAAAGTCAAGGAAATTGTTCAACAGATCTCATTTATTTAAGTCATTAAGCAATAGTTTGCAGCACTTT
AACAGCTTTTTGGTTATTTTTACATTTTAAGTGGATAACATATGGTATATAGCCAGACTGTACAGACATGTTTAAAAA
AACACACTGCTTAACCTATTAAATATGTGTTTAGAATTTTATAAGCAAATATAAATACTGTAAAAAGTCACTTTATTT
TATTTTTCAGCATTATGTACATAAATATGAAGAGGAAATTATCTTCAGGTTGATATCACAATCACTTTTCTTACTTTC
TGTCCATAGTACTTTTTCATGAAAGAAATTTGCTAAATAAGACATGAAAACAAGACTGGGTAGTTGTAGATTTCTGCT
TTTTAAATTACATTTGCTAATTTTAGATTATTTCACAATTTTAAGGAGCAAAATAGGTTCACGATTCATATCCAAATT
ATGCTTTGCAATTGGAAAAGGGTTTAAAATTTTATTTATATTTCTGGTAGTACCTGCACTAACTGAATTGAAGGTAGT
GCTTATGTTATTTTTGTTCTTTTTTTCTGACTTCGGTTTATGTTTTCATTTCTTTGGAGTAATGCTGCTCTAGATTGT
TCTAAATAGAATGTGGGCTTCATAATTTTTTTTTCCACAAAAACAGAGTAGTCAACTTATATAGTCAATTACATCAGG
ACATTTTGTGTTTCTTACAGAAGCAAACCATAGGCTCCTCTTTTCCTTAAAACTACTTAGATAAACTGTATTCGTGAA
CTGCATGCTGGAAAATGCTACTATTATGCTAAATAATGCTAACCAACATTTAAAATGTGCAAAACTAATAAAGATTAC
ATTTTTTATTTTATTGTTTGCCCAGTCACTTTTTGTTAACAGAATATTCTAATGATATGGAGATTTTTTACATTACAA
ATTGGGGGAGAAGGGGAGCGCGCGCGCACACACACACACACACACACACACACACACACACACACAGAGGCATACCCA
CGTTGACAACAAAACCTAGGGTAGATATGTCACTGGAGGTAGGGGGTAATGACCTCCCAGAATTACAAGCAGCAGGTG
TGTTCTCTGTTAGGAGGAAGAACTGGTGTCAGAGGATAGCTAGTGATTCTAGGAGGAAGAGAAGTATGGAAGCCAGAG
TGATGGTGGATGACCCCTTGAGCTATGAAAAGAAACCCTTAAATCATCATTTAAAAATTTAGAATTGCCATGTGTGTA
GGATACTGTGTTTGCTCCTCCAGAGCCACTCTCTCTGCTTCTGCATCATTCTGTGTGTCCCAGAAGGGTGACTTCTAC
ACATTGCAAAAATGGGCTCTCCTACCTTTGAGCTCCCAATTGGTTTGGCCAATGAGAAGCACCAGTGGGAAAGCACCA
GAGAGAGAAGATTGACATAGGAATATTTCTTCTCCAATTCCTTCTTTGCTGGGTTGGCACTGGACTCATTCCTCCCCG
AAAAGTCATACTCCAATCAGACTGCCCCTCATACAACTGAAGCTACTTTCTCTGGGGTCAGGTAATCACTCCTCCCCT
TGCTCCTTCAGGTCTGCTGCTGCATTGAGAGTGCTTTTGTATTCCTTGTAGCTTTCTCCTAACATTGCTGACACTTTT
GTAAATGTCCCCTTCATGAAATTCTTCTATATGCCTCATTTCAGCATGCCATCTGTCTCCTGCCTGGCTGACACAAGG
TGATTCAACAGCTCATGAAAGTCAGCAGGAAGCAAAGATGTGCCTTGCTTCAGCTTGGGGTCTTAATCTTGCTAACTT
TTGCAGATAAAGAAAAACAGTAACTGGGGGAACCACAGTGAAGTCCAGTGCAGAATTCACAGATATCATGGAAAGGTT
ACTCGGGTGGTCCAGATAGTAAAATTAACAGTCTAAATTAATCTATCTAAATTTCTGAGGAACGAGAAGCCTTCCCTT
GTCATCAGGTGAAGCCAGAAGAGGGAATATAGCCTCAACCAGAAAAGGGACAGTAATTAAAAGGCTTTTCCCATCCTT
GTACAATGGACTGACTTTGCCTCTTCATAACATCACAATCCTAAAGCAACACAACAATTAATTCTGATATATTAGTAG
CTGAAAAAAATTCCCATTTCCAACTAAGGTAGGTCAGAATTATAGGATAAACCCTGCAGACTTTTTATACTACCCATC
CACGCCATTACTCACTGTTACCTTTCCAAATACAAAGAGAAGAACTGGTAAAACATAATCATATAAATCTCCATATTC
ATTTTGAAATATTTGGCATGATATTTTCTGTGCTAAAAAGTAATTATTCTTCAAAGAATGATGAGGTCATGTCAGTAA
GACACAGGAACCAACTAGAAGGGGCTTCCCACTGGCCAAATCTGGGGCAAGTTGAGCATCAAAATAAATGATAGTAAA
AGATTATAATTCATTGAATAAGAATCAGCAAATACATACTGATGTAAGTAAATAAGGAAAAGTACAAATCTGTTTCTT
GCAGTTGAATGTTAATTAACAATTGTAGAAGAAATAACGGAGTTAGAAAAATCACTATTTGGCAATCACCCTAATGAC
AATTGATTCATACAAGAATCATCAATGAGTATTAAAACTCATGGGTGAAAGTTTGATGAGGAATAGGGTATTTATAGC
ATCTTAAAGTATCTCTTCTCTATTAAGTAGAAAATTTAAACAGAAGAAAGTATACTTTGGAGAAATACAGCAGACAAT
ACCTTCAAAGATATCATCAATTATGAGACCAACTGATACTATGTGCCTCCTGATAAGATATACTGAAAGGGCCACATT
ACTTCTTGGTACACAGTCAAAATTTTAAAACCAGAATCTAACTACAAGGAAAATCAAATTGAGGACACTCTATAAAAT
AAGTGGACTGAACTCCTTAAAAATGTCAATGTCATGAAAGACAAAGAAAGGCTAAAGAATTCCATGAGGTCAAAGAAC
TATGACAACTAAACACAATTCTGGATGGAATATCAAATTAAAAAATAACAGATAAATAATATTATTGGGAAAGTTGAA
TAAATTTGAATATGGACTGTTTATTAGTTATTAGTATTATAATAGTGTTAATTTTCCTAATTTTGTTAAGACTAGTGT
GCCTGTTCCATGAAAATAGAAAATGTTCTTATTCTCTGAAAATGCATGCTAAAGTATTTAGGGGTGAATGCAACAATG
TCTGCAGCTCATTCTTGAATCAGTTCAAAGAAAAATGAGTTACATTTATATATATATGTATGTATGTAAACAGACATA
GATAAAAGTATAGATGTGTGTGTGTCTTTAGAAAGGGGAGGATTTTTTTTTTTTTTTTGCTGTGTGTTACTGAAGTGC
CTATGTCTGCGTGTTCACACTATCATATTTTGTATGCCCTGGACTTTATAATTTCTACCTTCAAAATTAGATCTACTG
TTGGTAATTAATTCAATATATACTGGTTTTTTAACTACTATTCTCATTTCCTAGCAGTAATCTTCCTGAAAAGTCACA
GAAATGATTACATTCCTTGTTCTTCATAATAATCACTGTTTAATTAAAATAAGAATATTTTAGAAAAGATCTGCGGCA
TAGTGGTTAAGACCCCAGTATTTGATGCTAAACAGATCTGATTTGGATAACAGAAGGTGGCACTTTGCTGTTTAAGCT
GGGGACCAGACACTGTGGGTATAAATAGTAATTCCAAACACAGCTCCACAGAGCAGCACCCTTATGACAAGGTTTTCA
TATGTCTATAGTTAAGCCAGAAAATTAAGAATAATGCCATAAATATTTATAAAGCTGAACATATCCAAGTTAAAGACC
TTTATCCTGAAATTGTATCTTTTAGATTATTTTCTAAAGACTAATACCATTTAATGTTTAAATGTTCTTTGGAAATGA
TGGTGAGAATACGTGATAATGGGTCATTGGTTTTAATATTTTATTTAGCCAAGTGGAAAATTGGCAACCTGGTGTCGG
TCCTCCCATTTGTATTTTACTGGTGCATGAAATCCAAAAGTCTAGTAACCATTGGGACAGACAACTCTACTGCATAAG
TTTGTATGTTTGTATATCTGTATCACAAAGCCCAGACACTCGAACTATATAAACTTGTCGCACTAAAGACAGCAAATA
TGTCTGGTAATTGCATATTCTTCATGTGTGCACTGGAATTTCTTATTATATAAGAAAATAAATGTGTTTCTAAACCAC
CATGAA
SEQ ID NO: 19 >NP_001159435.1 SCN1A [organism = Homo sapiens] [GeneID = 6323]
[isoform = 1]
MEQTVLVPPGPDSFNFFTRESLAAIERRIAEEKAKNPKPDKKDDDENGPKPNSDLEAGKNLPFIYGDIPPEMVSEPLE
DLDPYYINKKTFIVLNKGKAIFRFSATSALYILTPFNPLRKIAIKILVHSLFSMLIMCTILTNCVFMTMSNPPDWTKN
VEYTFTGIYTFESLIKIIARGFCLEDFTFLRDPWNWLDFTVITFAYVTEFVDLGNVSALRTFRVLRALKTISVIPGLK
TIVGALIQSVKKLSDVMILTVFCLSVFALIGLQLFMGNLRNKCIQWPPTNASLEEHSIEKNITVNYNGTLINETVFEF
DWKSYIQDSRYHYFLEGELDALLCGNSSDAGQCPEGYMCVKAGRNPNYGYTSFDTFSWAFLSLFRLMTQDFWENLYQL
TLRAAGKTYMIFFVLVIFLGSFYLINLILAVVAMAYEEQNQATLEEAEQKEAEFQQMIEQLKKQQEAAQQAATATASE
HSREPSAAGRLSDSSSEASKLSSKSAKERRNRRKKRKQKEQSGGEEKDEDEFQKSESEDSIRRKGFRFSIEGNRLTYE
KRYSSPHQSLLSIRGSLESPRRNSRTSLFSFRGRAKDVGSENDFADDEHSTFEDNESRRDSLFVPRRHGERRNSNLSQ
TSRSSRMLAVFPANGKMHSTVDCNGVVSLVGGPSVPTSPVGQLLPEVIIDKPATDDNGTTTETEMRKRRSSSFHVSMD
FLEDPSQRQRAMSIASILTNTVEELEESRQKCPPCWYKFSNIFLIWDCSPYWLKVKHVVNLVVMDPFVDLAITICIVL
NTLFMAMEHYPMTDHFNNVLTVGNLVFTGIFTAEMELKIIAMDPYYYFQEGWNIFDGFIVTLSLVELGLANVEGLSVL
RSFRLLRVFKLAKSWPTLNMLIKIIGNSVGALGNLTLVLAIIVFIFAVVGMQLFGKSYKDCVCKIASDCQLPRWHMND
FFHSFLIVFRVLCGEWIETMWDCMEVAGQAMCLTVEMMVMVIGNLVVLNLFLALLLSSFSADNLAATDDDNEMNNLQI
AVDRMHKGVAYVKRKIYEFIQQSFIRKQKILDEIKPLDDLNNKKDSCMSNHTAEIGKDLDYLKDVNGTTSGIGTGSSV
EKYIIDESDYMSFINNPSLTVTVPIAVGESDFENLNTEDESSESDLEESKEKLNESSSSSEGSTVDIGAPVEEQPVVE
PEETLEPEACFTEGCVQRFKCCQINVEEGRGKQWWNLRRTCFRIVEHNWFETFIVEMILLSSGALAFEDIYIDQRKTI
KTMLEYADKVFTYIFILEMLLKWVAYGYQTYFTNAWCWLDFLIVDVSLVSLTANALGYSELGAIKSLRTLRALRPLRA
LSRFEGMRVVVNALLGAIPSIMNVLLVCLIFWLIFSIMGVNLFAGKFYHCINTTTGDRFDIEDVNNHTDCLKLIERNE
TARWKNVKVNFDNVGFGYLSLLQVATFKGWMDIMYAAVDSRNVELQPKYEESLYMYLYFVIFIIFGSFFTLNLFIGVI
IDNFNQQKKKFGGQDIFMTEEQKKYYNAMKKLGSKKPQKPIPRPGNKFQGMVFDFVTRQVEDISIMILICLNMVTMMV
ETDDQSEYVTTILSRINLVFIVLFTGECVLKLISLRHYYFTIGWNIFDFVVVILSIVGMFLAELIEKYFVSPTLFRVI
RLARIGRILRLIKGAKGIRTLLFALMMSLPALFNIGLLLFLVMFIYAIFGMSNFAYVKREVGIDDMENFETEGNSMIC
LFQITTSAGWDGLLAPILNSKPPDCDPNKVNPGSSVKGDCGNPSVGIFFFVSYIIISELVVVNMYIAVILENESVATE
ESAEPLSEDDFEMFYEVWEKFDPDATQFMEFEKLSQFAAALEPPLNLPQPNKLQLIAMDLPMVSGDRIHCLDILFAFT
KRVLGESGEMDALRIQMEERFMASNPSKVSYQPITTTLKRKQEEVSAVIIQRAYRRHLLKRTVKQASFTYNKNKIKGG
ANLLIKEDMIIDRINENSITEKTDLTMSTAACPPSYDRVTKPIVEKHEQEGKDEKAKGK
SEQ ID NO: 20 >NP_001159436.1 SCN1A [organism = Homo sapiens] [GeneID = 6323]
[isoform = 3]
MEQTVLVPPGPDSFNFFTRESLAAIERRIAEEKAKNPKPDKKDDDENGPKPNSDLEAGKNLPFIYGDIPPEMVSEPLE
DLDPYYINKKTFIVLNKGKAIFRFSATSALYILTPFNPLRKIAIKILVHSLFSMLIMCTILTNCVFMTMSNPPDWTKN
VEYTFTGIYTFESLIKIIARGFCLEDFTFLRDPWNWLDFTVITFAYVTEFVDLGNVSALRTFRVLRALKTISVIPGLK
TIVGALIQSVKKLSDVMILTVFCLSVFALIGLQLFMGNLRNKCIQWPPTNASLEEHSIEKNITVNYNGTLINETVFEF
DWKSYIQDSRYHYFLEGELDALLCGNSSDAGQCPEGYMCVKAGRNPNYGYTSFDTFSWAFLSLFRLMTQDFWENLYQL
TLRAAGKTYMIFFVLVIFLGSFYLINLILAVVAMAYEEQNQATLEEAEQKEAEFQQMIEQLKKQQEAAQQAATATASE
HSREPSAAGRLSDSSSEASKLSSKSAKERRNRRKKRKQKEQSGGEEKDEDEFQKSESEDSIRRKGFRFSIEGNRLTYE
KRYSSPHQSLLSIRGSLFSPRRNSRTSLESFRGRAKDVGSENDFADDEHSTFEDNESRRDSLFVPRRHGERRNSNLSQ
TSRSSRMLAVFPANGKMHSTVDCNGVVSLGTTTETEMRKRRSSSFHVSMDFLEDPSQRQRAMSIASILTNTVEELEES
RQKCPPCWYKFSNIFLIWDCSPYWLKVKHVVNLVVMDPFVDLAITICIVLNTLFMAMEHYPMTDHFNNVLTVGNLVFT
GIFTAEMFLKIIAMDPYYYFQEGWNIFDGFIVTLSLVELGLANVEGLSVLRSFRLLRVFKLAKSWPTLNMLIKIIGNS
VGALGNLTLVLAIIVFIFAVVGMQLFGKSYKDCVCKIASDCQLPRWHMNDFFHSFLIVFRVLCGEWIETMWDCMEVAG
QAMCLTVFMMVMVIGNLVVLNLFLALLLSSFSADNLAATDDDNEMNNLQIAVDRMHKGVAYVKRKIYEFIQQSFIRKQ
KILDEIKPLDDLNNKKDSCMSNHTAEIGKDLDYLKDVNGTTSGIGTGSSVEKYIIDESDYMSFINNPSLTVTVPIAVG
ESDFENLNTEDESSESDLEESKEKLNESSSSSEGSTVDIGAPVEEQPVVEPEETLEPEACFTEGCVQRFKCCQINVEE
GRGKQWWNLRRTCFRIVEHNWFETFIVEMILLSSGALAFEDIYIDQRKTIKTMLEYADKVFTYIFILEMLLKWVAYGY
QTYFTNAWCWLDFLIVDVSLVSLTANALGYSELGAIKSLRTLRALRPLRALSRFEGMRVVVNALLGAIPSIMNVLLVC
LIFWLIFSIMGVNLFAGKFYHCINTTTGDRFDIEDVNNHTDCLKLIERNETARWKNVKVNFDNVGFGYLSLLQVATFK
GWMDIMYAAVDSRNVELQPKYEESLYMYLYFVIFIIFGSFFTLNLFIGVIIDNFNQQKKKFGGQDIFMTEEQKKYYNA
MKKLGSKKPQKPIPRPGNKFQGMVFDFVTRQVFDISIMILICLNMVTMMVETDDQSEYVTTILSRINLVFIVLFTGEC
VLKLISLRHYYFTIGWNIFDFVVVILSIVGMFLAELIEKYFVSPTLFRVIRLARIGRILRLIKGAKGIRTLLFALMMS
LPALFNIGLLLFLVMFIYAIFGMSNFAYVKREVGIDDMENFETFGNSMICLFQITTSAGWDGLLAPILNSKPPDCDPN
KVNPGSSVKGDCGNPSVGIFFFVSYIIISFLVVVNMYIAVILENFSVATEESAEPLSEDDFEMFYEVWEKFDPDATQF
MEFEKLSQFAAALEPPLNLPQPNKLQLIAMDLPMVSGDRIHCLDILFAFTKRVLGESGEMDALRIQMEERFMASNPSK
VSYQPITTTLKRKQEEVSAVIIQRAYRRHLLKRTVKQASFTYNKNKIKGGANLLIKEDMIIDRINENSITEKTDLTMS
TAACPPSYDRVTKPIVEKHEQEGKDEKAKGK
SEQ ID NO: 21 >NP_001340878.1 SCN1A [organism = Homo sapiens] [GeneID = 6323]
[isoform = 2]
MEQTVLVPPGPDSFNFFTRESLAAIERRIAEEKAKNPKPDKKDDDENGPKPNSDLEAGKNLPFIYGDIPPEMVSEPLE
DLDPYYINKKTFIVLNKGKAIFRFSATSALYILTPFNPLRKIAIKILVHSLFSMLIMCTILTNCVFMTMSNPPDWTKN
VEYTFTGIYTFESLIKIIARGFCLEDFTFLRDPWNWLDFTVITFAYVTEFVDLGNVSALRTFRVLRALKTISVIPGLK
TIVGALIQSVKKLSDVMILTVFCLSVFALIGLQLFMGNLRNKCIQWPPTNASLEEHSIEKNITVNYNGTLINETVFEF
DWKSYIQDSRYHYFLEGELDALLCGNSSDAGQCPEGYMCVKAGRNPNYGYTSEDTESWAFLSLFRLMTQDEWENLYQL
TLRAAGKTYMIFFVLVIFLGSFYLINLILAVVAMAYEEQNQATLEEAEQKEAEFQQMIEQLKKQQEAAQQAATATASE
HSREPSAAGRLSDSSSEASKLSSKSAKERRNRRKKRKQKEQSGGEEKDEDEFQKSESEDSIRRKGFRFSIEGNRLTYE
KRYSSPHQSLLSIRGSLFSPRRNSRTSLESFRGRAKDVGSENDFADDEHSTFEDNESRRDSLFVPRRHGERRNSNLSQ
TSRSSRMLAVFPANGKMHSTVDCNGVVSLVGGPSVPTSPVGQLLPEGTTTETEMRKRRSSSFHVSMDFLEDPSQRQRA
MSIASILTNTVEELEESRQKCPPCWYKFSNIFLIWDCSPYWLKVKHVVNLVVMDPFVDLAITICIVLNTLFMAMEHYP
MTDHENNVLTVGNLVFTGIFTAEMFLKIIAMDPYYYFQEGWNIFDGFIVTLSLVELGLANVEGLSVLRSFRLLRVFKL
AKSWPTLNMLIKIIGNSVGALGNLTLVLAIIVFIFAVVGMQLFGKSYKDCVCKIASDCQLPRWHMNDFFHSFLIVERV
LCGEWIETMWDCMEVAGQAMCLTVFMMVMVIGNLVVLNLFLALLLSSFSADNLAATDDDNEMNNLQIAVDRMHKGVAY
VKRKIYEFIQQSFIRKQKILDEIKPLDDLNNKKDSCMSNHTAEIGKDLDYLKDVNGTTSGIGTGSSVEKYIIDESDYM
SFINNPSLTVTVPIAVGESDFENLNTEDESSESDLEESKEKLNESSSSSEGSTVDIGAPVEEQPVVEPEETLEPEACE
TEGCVQRFKCCQINVEEGRGKQWWNLRRTCFRIVEHNWFETFIVEMILLSSGALAFEDIYIDQRKTIKTMLEYADKVF
TYIFILEMLLKWVAYGYQTYFTNAWCWLDFLIVDVSLVSLTANALGYSELGAIKSLRTLRALRPLRALSRFEGMRVVV
NALLGAIPSIMNVLLVCLIFWLIFSIMGVNLFAGKFYHCINTTTGDREDIEDVNNHTDCLKLIERNETARWKNVKVNF
DNVGFGYLSLLQVATFKGWMDIMYAAVDSRNVELQPKYEESLYMYLYFVIFIIFGSFFTLNLFIGVIIDNFNQQKKKF
GGQDIFMTEEQKKYYNAMKKLGSKKPQKPIPRPGNKFQGMVFDFVTRQVEDISIMILICLNMVTMMVETDDQSEYVTT
ILSRINLVFIVLFTGECVLKLISLRHYYFTIGWNIFDFVVVILSIVGMFLAELIEKYFVSPTLERVIRLARIGRILRL
IKGAKGIRTLLFALMMSLPALFNIGLLLFLVMFIYAIFGMSNFAYVKREVGIDDMENFETFGNSMICLFQITTSAGWD
GLLAPILNSKPPDCDPNKVNPGSSVKGDCGNPSVGIFFFVSYIIISFLVVVNMYIAVILENFSVATEESAEPLSEDDE
EMFYEVWEKFDPDATQFMEFEKLSQFAAALEPPLNLPQPNKLQLIAMDLPMVSGDRIHCLDILFAFTKRVLGESGEMD
ALRIQMEERFMASNPSKVSYQPITTTLKRKQEEVSAVIIQRAYRRHLLKRTVKQASFTYNKNKIKGGANLLIKEDMII
DRINENSITEKTDLTMSTAACPPSYDRVTKPIVEKHEQEGKDEKAKGK
SEQ ID NO: 22 >NP_001340883.1 SCN1A [organism = Homo sapiens] [GeneID = 6323]
[isoform = 4]
MEQTVLVPPGPDSFNFFTRESLAAIERRIAEEKAKNPKPDKKDDDENGPKPNSDLEAGKNLPFIYGDIPPEMVSEPLE
DLDPYYINKKTFIVLNKGKAIFRFSATSALYILTPFNPLRKIAIKILVHSLESMLIMCTILTNCVFMTMSNPPDWTKN
VEYTFTGIYTFESLIKIIARGFCLEDFTFLRDPWNWLDFTVITFAYVTEFVDLGNVSALRTFRVLRALKTISVIPGLK
TIVGALIQSVKKLSDVMILTVFCLSVFALIGLQLFMGNLRNKCIQWPPTNASLEEHSIEKNITVNYNGTLINETVFEF
DWKSYIQDSRYHYFLEGFLDALLCGNSSDAGQCPEGYMCVKAGRNPNYGYTSFDTFSWAFLSLFRLMTQDFWENLYQL
TLRAAGKTYMIFFVLVIFLGSFYLINLILAVVAMAYEEQNQATLEEAEQKEAEFQQMIEQLKKQQEAAQAATATASEH
SREPSAAGRLSDSSSEASKLSSKSAKERRNRRKKRKQKEQSGGEEKDEDEFQKSESEDSIRRKGFRFSIEGNRLTYEK
RYSSPHQSLLSIRGSLFSPRRNSRTSLFSFRGRAKDVGSENDFADDEHSTFEDNESRRDSLFVPRRHGERRNSNLSQT
SRSSRMLAVFPANGKMHSTVDCNGVVSLVGGPSVPTSPVGQLLPEGTTTETEMRKRRSSSFHVSMDFLEDPSQRQRAM
SIASILTNTVEELEESRQKCPPCWYKFSNIFLIWDCSPYWLKVKHVVNLVVMDPFVDLAITICIVLNTLFMAMEHYPM
TDHFNNVLTVGNLVFTGIFTAEMFLKIIAMDPYYYFQEGWNIFDGFIVTLSLVELGLANVEGLSVLRSFRLLRVFKLA
KSWPTLNMLIKIIGNSVGALGNLTLVLAIIVFIFAVVGMQLFGKSYKDCVCKIASDCQLPRWHMNDFFHSFLIVERVL
CGEWIETMWDCMEVAGQAMCLTVFMMVMVIGNLVVLNLFLALLLSSFSADNLAATDDDNEMNNLQIAVDRMHKGVAYV
KRKIYEFIQQSFIRKQKILDEIKPLDDLNNKKDSCMSNHTAEIGKDLDYLKDVNGTTSGIGTGSSVEKYIIDESDYMS
FINNPSLTVTVPIAVGESDFENLNTEDESSESDLEESKEKLNESSSSSEGSTVDIGAPVEEQPVVEPEETLEPEACFT
EGCVQRFKCCQINVEEGRGKQWWNLRRTCFRIVEHNWFETFIVEMILLSSGALAFEDIYIDQRKTIKTMLEYADKVFT
YIFILEMLLKWVAYGYQTYFTNAWCWLDFLIVDVSLVSLTANALGYSELGAIKSLRTLRALRPLRALSRFEGMRVVVN
ALLGAIPSIMNVLLVCLIFWLIFSIMGVNLFAGKFYHCINTTTGDRFDIEDVNNHTDCLKLIERNETARWKNVKVNED
NVGFGYLSLLQVATFKGWMDIMYAAVDSRNVELQPKYEESLYMYLYFVIFIIFGSFFTLNLFIGVIIDNFNQQKKKFG
GQDIFMTEEQKKYYNAMKKLGSKKPQKPIPRPGNKFQGMVFDFVTRQVFDISIMILICLNMVTMMVETDDQSEYVTTI
LSRINLVFIVLFTGECVLKLISLRHYYFTIGWNIFDFVVVILSIVGMFLAELIEKYFVSPTLERVIRLARIGRILRLI
KGAKGIRTLLFALMMSLPALFNIGLLLFLVMFIYAIFGMSNFAYVKREVGIDDMENFETFGNSMICLFQITTSAGWDG
LLAPILNSKPPDCDPNKVNPGSSVKGDCGNPSVGIFFFVSYIIISFLVVVNMYIAVILENFSVATEESAEPLSEDDFE
MFYEVWEKFDPDATQFMEFEKLSQFAAALEPPLNLPQPNKLQLIAMDLPMVSGDRIHCLDILFAFTKRVLGESGEMDA
LRIQMEERFMASNPSKVSYQPITTTLKRKQEEVSAVIIQRAYRRHLLKRTVKQASFTYNKNKIKGGANLLIKEDMIID
RINENSITEKTDLTMSTAACPPSYDRVTKPIVEKHEQEGKDEKAKGK
SEQ ID NO: 23 >NP_001340889.1 SCN1A [organism = Homo sapiens] [GeneID = 6323]
[isoform = 5]
MEQTVLVPPGPDSFNFFTRESLAAIERRIAEEKAKNPKPDKKDDDENGPKPNSDLEAGKNLPFIYGDIPPEMVSEPLE
DLDPYYINKKTFIVLNKGKAIFRFSATSALYILTPFNPLRKIAIKILVHSLESMLIMCTILTNCVFMTMSNPPDWTKN
VEYTFTGIYTFESLIKIIARGFCLEDFTFLRDPWNWLDFTVITFAYVTEFVDLGNVSALRTFRVLRALKTISVIPGLK
TIVGALIQSVKKLSDVMILTVFCLSVFALIGLQLFMGNLRNKCIQWPPTNASLEEHSIEKNITVNYNGTLINETVFEF
DWKSYIQDSRYHYFLEGFLDALLCGNSSDAGQCPEGYMCVKAGRNPNYGYTSFDTFSWAFLSLFRLMTQDFWENLYQL
TLRAAGKTYMIFFVLVIFLGSFYLINLILAVVAMAYEEQNQATLEEAEQKEAEFQQMIEQLKKQQEAAQAATATASEH
SREPSAAGRLSDSSSEASKLSSKSAKERRNRRKKRKQKEQSGGEEKDEDEFQKSESEDSIRRKGFRESIEGNRLTYEK
RYSSPHQSLLSIRGSLESPRRNSRTSLFSFRGRAKDVGSENDFADDEHSTFEDNESRRDSLFVPRRHGERRNSNLSQT
SRSSRMLAVFPANGKMHSTVDCNGVVSLGTTTETEMRKRRSSSFHVSMDFLEDPSQRQRAMSIASILTNTVEELEESR
QKCPPCWYKFSNIFLIWDCSPYWLKVKHVVNLVVMDPFVDLAITICIVLNTLFMAMEHYPMTDHENNVLTVGNLVETG
IFTAEMFLKIIAMDPYYYFQEGWNIFDGFIVTLSLVELGLANVEGLSVLRSFRLLRVFKLAKSWPTLNMLIKIIGNSV
GALGNLTLVLAIIVFIFAVVGMQLFGKSYKDCVCKIASDCQLPRWHMNDFFHSFLIVFRVLCGEWIETMWDCMEVAGQ
AMCLTVFMMVMVIGNLVVLNLFLALLLSSFSADNLAATDDDNEMNNLQIAVDRMHKGVAYVKRKIYEFIQQSFIRKQK
ILDEIKPLDDLNNKKDSCMSNHTAEIGKDLDYLKDVNGTTSGIGTGSSVEKYIIDESDYMSFINNPSLTVTVPIAVGE
SDFENLNTEDESSESDLEESKEKLNESSSSSEGSTVDIGAPVEEQPVVEPEETLEPEACFTEGCVQRFKCCQINVEEG
RGKQWWNLRRTCFRIVEHNWFETFIVEMILLSSGALAFEDIYIDQRKTIKTMLEYADKVETYIFILEMLLKWVAYGYQ
TYFTNAWCWLDFLIVDVSLVSLTANALGYSELGAIKSLRTLRALRPLRALSRFEGMRVVVNALLGAIPSIMNVLLVCL
IFWLIFSIMGVNLFAGKFYHCINTTTGDRFDIEDVNNHTDCLKLIERNETARWKNVKVNFDNVGFGYLSLLQVATEKG
WMDIMYAAVDSRNVELQPKYEESLYMYLYFVIFIIFGSFFTLNLFIGVIIDNENQQKKKFGGQDIFMTEEQKKYYNAM
KKLGSKKPQKPIPRPGNKFQGMVFDFVTRQVEDISIMILICLNMVTMMVETDDQSEYVTTILSRINLVFIVLFTGECV
LKLISLRHYYFTIGWNIFDFVVVILSIVGMFLAELIEKYFVSPTLFRVIRLARIGRILRLIKGAKGIRTLLFALMMSL
PALFNIGLLLFLVMFIYAIFGMSNFAYVKREVGIDDMENFETFGNSMICLFQITTSAGWDGLLAPILNSKPPDCDPNK
VNPGSSVKGDCGNPSVGIFFFVSYIIISFLVVVNMYIAVILENFSVATEESAEPLSEDDFEMFYEVWEKFDPDATQFM
EFEKLSQFAAALEPPLNLPQPNKLQLIAMDLPMVSGDRIHCLDILFAFTKRVLGESGEMDALRIQMEERFMASNPSKV
SYQPITTTLKRKQEEVSAVIIQRAYRRHLLKRTVKQASFTYNKNKIKGGANLLIKEDMIIDRINENSITEKTDLTMST
AACPPSYDRVTKPIVEKHEQEGKDEKAKGK
SEQ ID NO: 24 >NP_001340890.1 SCN1A [organism = Homo sapiens] [GeneID = 6323]
[isoform = 6]
MFLKIIAMDPYYYFQEGWNIFDGFIVTLSLVELGLANVEGLSVLRSFRLLRVFKLAKSWPTLNMLIKIIGNSVGALGN
LTLVLAIIVFIFAVVGMQLFGKSYKDCVCKIASDCQLPRWHMNDFFHSFLIVFRVLCGEWIETMWDCMEVAGQAMCLT
VFMMVMVIGNLVVLNLFLALLLSSFSADNLAATDDDNEMNNLQIAVDRMHKGVAYVKRKIYEFIQQSFIRKQKILDEI
KPLDDLNNKKDSCMSNHTAEIGKDLDYLKDVNGTTSGIGTGSSVEKYIIDESDYMSFINNPSLTVTVPIAVGESDFEN
LNTEDESSESDLEESKEKLNESSSSSEGSTVDIGAPVEEQPVVEPEETLEPEACFTEGCVQRFKCCQINVEEGRGKQW
WNLRRTCFRIVEHNWFETFIVEMILLSSGALAFEDIYIDQRKTIKTMLEYADKVFTYIFILEMLLKWVAYGYQTYFTN
AWCWLDFLIVDVSLVSLTANALGYSELGAIKSLRTLRALRPLRALSRFEGMRVVVNALLGAIPSIMNVLLVCLIFWLI
FSIMGVNLFAGKFYHCINTTTGDRFDIEDVNNHTDCLKLIERNETARWKNVKVNFDNVGFGYLSLLQVATFKGWMDIM
YAAVDSRNVELQPKYEESLYMYLYFVIFIIFGSFFTLNLFIGVIIDNFNQQKKKFGGQDIFMTEEQKKYYNAMKKLGS
KKPQKPIPRPGNKFQGMVFDFVTRQVFDISIMILICLNMVTMMVETDDQSEYVTTILSRINLVFIVLFTGECVLKLIS
LRHYYFTIGWNIFDFVVVILSIVGMFLAELIEKYFVSPTLFRVIRLARIGRILRLIKGAKGIRTLLFALMMSLPALEN
IGLLLFLVMFIYAIFGMSNFAYVKREVGIDDMENFETFGNSMICLFQITTSAGWDGLLAPILNSKPPDCDPNKVNPGS
SVKGDCGNPSVGIFFFVSYIIISFLVVVNMYIAVILENFSVATEESAEPLSEDDFEMFYEVWEKFDPDATQFMEFEKL
SQFAAALEPPLNLPQPNKLQLIAMDLPMVSGDRIHCLDILFAFTKRVLGESGEMDALRIQMEERFMASNPSKVSYQPI
TTTLKRKQEEVSAVIIQRAYRRHLLKRTVKQASFTYNKNKIKGGANLLIKEDMIIDRINENSITEKTDLTMSTAACPP
SYDRVTKPIVEKHEQEGKDEKAKGK
SEQ ID NO: 25 >NM_001386139.1 MECP2 [organism = Homo sapiens] [GeneID = 4204]
[transcript = 10]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGGAAGAAAAGTCAGAAGACCAGGACCTCCAGGGCCTCAAGGA
CAAACCCCTCAAGTTTAAAAAGGTGAAGAAAGATAAGAAAGAAGAGAAAGAGGGCAAGCATGAGCCCGTGCAGCCATC
AGCCCACCACTCTGCTGAGCCCGCAGAGGCAGGCAAAGCAGAGACATCAGAAGGGTCAGGCTCCGCCCCGGCTGTGCC
GGAAGCTTCTGCCTCCCCCAAACAGCGGCGCTCCATCATCCGTGACCGGGGACCCATGTATGATGACCCCACCCTGCC
TGAAGGCTGGACACGGAAGCTTAAGCAAAGGAAATCTGGCCGCTCTGCTGGGAAGTATGATGTGTATTTGATCAATCC
CCAGGGAAAAGCCTTTCGCTCTAAAGTGGAGTTGATTGCGTACTTCGAAAAGCTCCAGGAACTGGCAGAGGCCGGGGA
CGCCCCAAAGGGAGCGGCACCACGAGACCCAAGGCGGCCACGTCAGAGGGTGTGCAGGTGAAAAGGGTCCTGGAGAAA
AGTCCTGGGAAGCTCCTTGTCAAGATGCCTTTTCAAACTTCGCCAGGGGGCAAGGCTGAGGGGGGTGGGGCCACCACA
TCCACCCAGGTCATGGTGATCAAACGCCCCGGCAGGAAGCGAAAAGCTGAGGCCGACCCTCAGGCCATTCCCAAGAAA
CGGGGCCGAAAGCCGGGGAGTGTGGTGGCAGCCGCTGCCGCCGAGGCCAAAAAGAAAGCCGTGAAGGAGTCTTCTATC
CGATCTGTGCAGGAGACCGTACTCCCCATCAAGAAGCGCAAGACCCGGGAGACGGTCAGCATCGAGGTCAAGGAAGTG
GTGAAGCCCCTGCTGGTGTCCACCCTCGGTGAGAAGAGCGGGAAAGGACTGAAGACCTGTAAGAGCCCTGGGCGGAAA
AGCAAGGAGAGCAGCCCCAAGGGGCGCAGCAGCAGCGCCTCCTCACCCCCCAAGAAGGAGCACCACCACCATCACCAC
CACTCAGAGTCCCCAAAGGCCCCCGTGCCACTGCTCCCACCCCTGCCCCCACCTCCACCTGAGCCCGAGAGCTCCGAG
GACCCCACCAGCCCCCCTGAGCCCCAGGACTTGAGCAGCAGCGTCTGCAAAGAGGAGAAGATGCCCAGAGGAGGCTCA
CTGGAGAGCGACGGCTGCCCCAAGGAGCCAGCTAAGACTCAGCCCGCGGTTGCCACCGCCGCCACGGCCGCAGAAAAG
TACAAACACCGAGGGGAGGGAGAGCGCAAAGACATTGTTTCATCCTCCATGCCAAGGCCAAACAGAGAGGAGCCTGTG
GACAGCCGGACGCCCGTGACCGAGAGAGTTAGCTGACTTTACACGGAGCGGATTGCAAAGCAAACCAACAAGAATAAA
GGCAGCTGTTGTCTCTTCTCCTTATGGGTAGGGCTCTGACAAAGCTTCCCGATTAACTGAAATAAAAAATATTTTTTT
TTCTTTCAGTAAACTTAGAGTTTCGTGGCTTCAGGGTGGGAGTAGTTGGAGCATTGGGGATGTTTTTCTTACCGACAA
GCACAGTCAGGTTGAAGACCTAACCAGGGCCAGAAGTAGCTTTGCACTTTTCTAAACTAGGCTCCTTCAACAAGGCTT
GCTGCAGATACTACTGACCAGACAAGCTGTTGACCAGGCACCTCCCCTCCCGCCCAAACCTTTCCCCCATGTGGTCGT
TAGAGACAGAGCGACAGAGCAGTTGAGAGGACACTCCCGTTTTCGGTGCCATCAGTGCCCCGTCTACAGCTCCCCCAG
CTCCCCCCACCTCCCCCACTCCCAACCACGTTGGGACAGGGAGGTGTGAGGCAGGAGAGACAGTTGGATTCTTTAGAG
AAGATGGATATGACCAGTGGCTATGGCCTGTGCGATCCCACCCGTGGTGGCTCAAGTCTGGCCCCACACCAGCCCCAA
TCCAAAACTGGCAAGGACGCTTCACAGGACAGGAAAGTGGCACCTGTCTGCTCCAGCTCTGGCATGGCTAGGAGGGGG
GAGTCCCTTGAACTACTGGGTGTAGACTGGCCTGAACCACAGGAGAGGATGGCCCAGGGTGAGGTGGCATGGTCCATT
CTCAAGGGACGTCCTCCAACGGGTGGCGCTAGAGGCCATGGAGGCAGTAGGACAAGGTGCAGGCAGGCTGGCCTGGGG
TCAGGCCGGGCAGAGCACAGCGGGGTGAGAGGGATTCCTAATCACTCAGAGCAGTCTGTGACTTAGTGGACAGGGGAG
GGGGCAAAGGGGGAGGAGAAGAAAATGTTCTTCCAGTTACTTTCCAATTCTCCTTTAGGGACAGCTTAGAATTATTTG
CACTATTGAGTCTTCATGTTCCCACTTCAAAACAAACAGATGCTCTGAGAGCAAACTGGCTTGAATTGGTGACATTTA
GTCCCTCAAGCCACCAGATGTGACAGTGTTGAGAACTACCTGGATTTGTATATATACCTGCGCTTGTTTTAAAGTGGG
CTCAGCACATAGGGTTCCCACGAAGCTCCGAAACTCTAAGTGTTTGCTGCAATTTTATAAGGACTTCCTGATTGGTTT
CTCTTCTCCCCTTCCATTTCTGCCTTTTGTTCATTTCATCCTTTCACTTCTTTCCCTTCCTCCATCCTCCTCCTTCCT
AGTTCATCCCTTCTCTTCCAGGCAGCCGCGGTGCCCAACCACACTTGTCGGCTCCAGTCCCCAGAACTCTGCCTGCCC
TTTGTCCTCCTGCTGCCAGTACCAGCCCCACCCTGTTTTGAGCCCTGAGGAGGCCTTGGGCTCTGCTGAGTCCGACCT
GGCCTGTCTGTGAAGAGCAAGAGAGCAGCAAGGTCTTGCTCTCCTAGGTAGCCCCCTCTTCCCTGGTAAGAAAAAGCA
AAAGGCATTTCCCACCCTGAACAACGAGCCTTTTCACCCTTCTACTCTAGAGAAGTGGACTGGAGGAGCTGGGCCCGA
TTTGGTAGTTGAGGAAAGCACAGAGGCCTCCTGTGGCCTGCCAGTCATCGAGTGGCCCAACAGGGGCTCCATGCCAGC
CGACCTTGACCTCACTCAGAAGTCCAGAGTCTAGCGTAGTGCAGCAGGGCAGTAGCGGTACCAATGCAGAACTCCCAA
GACCCGAGCTGGGACCAGTACCTGGGTCCCCAGCCCTTCCTCTGCTCCCCCTTTTCCCTCGGAGTTCTTCTTGAATGG
CAATGTTTTGCTTTTGCTCGATGCAGACAGGGGGCCAGAACACCACACATTTCACTGTCTGTCTGGTCCATAGCTGTG
GTGTAGGGGCTTAGAGGCATGGGCTTGCTGTGGGTTTTTAATTGATCAGTTTTCATGTGGGATCCCATCTTTTTAACC
TCTGTTCAGGAAGTCCTTATCTAGCTGCATATCTTCATCATATTGGTATATCCTTTTCTGTGTTTACAGAGATGTCTC
TTATATCTAAATCTGTCCAACTGAGAAGTACCTTATCAAAGTAGCAAATGAGACAGCAGTCTTATGCTTCCAGAAACA
CCCACAGGCATGTCCCATGTGAGCTGCTGCCATGAACTGTCAAGTGTGTGTTGTCTTGTGTATTTCAGTTATTGTCCC
TGGCTTCCTTACTATGGTGTAATCATGAAGGAGTGAAACATCATAGAAACTGTCTAGCACTTCCTTGCCAGTCTTTAG
TGATCAGGAACCATAGTTGACAGTTCCAATCAGTAGCTTAAGAAAAAACCGTGTTTGTCTCTTCTGGAATGGTTAGAA
GTGAGGGAGTTTGCCCCGTTCTGTTTGTAGAGTCTCATAGTTGGACTTTCTAGCATATATGTGTCCATTTCCTTATGC
TGTAAAAGCAAGTCCTGCAACCAAACTCCCATCAGCCCAATCCCTGATCCCTGATCCCTTCCACCTGCTCTGCTGATG
ACCCCCCCAGCTTCACTTCTGACTCTTCCCCAGGAAGGGAAGGGGGGTCAGAAGAGAGGGTGAGTCCTCCAGAACTCT
TCCTCCAAGGACAGAAGGCTCCTGCCCCCATAGTGGCCTCGAACTCCTGGCACTACCAAAGGACACTTATCCACGAGA
GCGCAGCATCCGACCAGGTTGTCACTGAGAAGATGTTTATTTTGGTCAGTTGGGTTTTTATGTATTATACTTAGTCAA
ATGTAATGTGGCTTCTGGAATCATTGTCCAGAGCTGCTTCCCCGTCACCTGGGCGTCATCTGGTCCTGGTAAGAGGAG
TGCGTGGCCCACCAGGCCCCCCTGTCACCCATGACAGTTCATTCAGGGCCGATGGGGCAGTCGTGGTTGGGAACACAG
CATTTCAAGCGTCACTTTATTTCATTCGGGCCCCACCTGCAGCTCCCTCAAAGAGGCAGTTGCCCAGCCTCTTTCCCT
TCCAGTTTATTCCAGAGCTGCCAGTGGGGCCTGAGGCTCCTTAGGGTTTTCTCTCTATTTCCCCCTTTCTTCCTCATT
CCCTCGTCTTTCCCAAAGGCATCACGAGTCAGTCGCCTTTCAGCAGGCAGCCTTGGCGGTTTATCGCCCTGGCAGGCA
GGGGCCCTGCAGCTCTCATGCTGCCCCTGCCTTGGGGTCAGGTTGACAGGAGGTTGGAGGGAAAGCCTTAAGCTGCAG
GATTCTCACCAGCTGTGTCCGGCCCAGTTTTGGGGTGTGACCTCAATTTCAATTTTGTCTGTACTTGAACATTATGAA
GATGGGGGCCTCTTTCAGTGAATTTGTGAACAGCAGAATTGACCGACAGCTTTCCAGTACCCATGGGGCTAGGTCATT
AAGGCCACATCCACAGTCTCCCCCACCCTTGTTCCAGTTGTTAGTTACTACCTCCTCTCCTGACAATACTGTATGTCG
TCGAGCTCCCCCCAGGTCTACCCCTCCCGGCCCTGCCTGCTGGTGGGCTTGTCATAGCCAGTGGGATTGCCGGTCTTG
ACAGCTCAGTGAGCTGGAGATACTTGGTCACAGCCAGGCGCTAGCACAGCTCCCTTCTGTTGATGCTGTATTCCCATA
TCAAAAGACACAGGGGACACCCAGAAACGCCACATCCCCCAATCCATCAGTGCCAAACTAGCCAACGGCCCCAGCTTC
TCAGCTCGCTGGATGGCGGAAGCTGCTACTCGTGAGCGCCAGTGCGGGTGCAGACAATCTTCTGTTGGGTGGCATCAT
TCCAGGCCCGAAGCATGAACAGTGCACCTGGGACAGGGAGCAGCCCCAAATTGTCACCTGCTTCTCTGCCCAGCTTTT
CATTGCTGTGACAGTGATGGCGAAAGAGGGTAATAACCAGACACAAACTGCCAAGTTGGGTGGAGAAAGGAGTTTCTT
TAGCTGACAGAATCTCTGAATTTTAAATCACTTAGTAAGCGGCTCAAGCCCAGGAGGGAGCAGAGGGATACGAGCGGA
GTCCCCTGCGCGGGACCATCTGGAATTGGTTTAGCCCAAGTGGAGCCTGACAGCCAGAACTCTGTGTCCCCCGTCTAA
CCACAGCTCCTTTTCCAGAGCATTCCAGTCAGGCTCTCTGGGCTGACTGGGCCAGGGGAGGTTACAGGTACCAGTTCT
TTAAGAAGATCTTTGGGCATATACATTTTTAGCCTGTGTCATTGCCCCAAATGGATTCCTGTTTCAAGTTCACACCTG
CAGATTCTAGGACCTGTGTCCTAGACTTCAGGGAGTCAGCTGTTTCTAGAGTTCCTACCATGGAGTGGGTCTGGAGGA
CCTGCCCGGTGGGGGGGCAGAGCCCTGCTCCCTCCGGGTCTTCCTACTCTTCTCTCTGCTCTGACGGGATTTGTTGAT
TCTCTCCATTTTGGTGTCTTTCTCTTTTAGATATTGTATCAATCTTTAGAAAAGGCATAGTCTACTTGTTATAAATCG
TTAGGATACTGCCTCCCCCAGGGTCTAAAATTACATATTAGAGGGGAAAAGCTGAACACTGAAGTCAGTTCTCAACAA
TTTAGAAGGAAAACCTAGAAAACATTTGGCAGAAAATTACATTTCGATGTTTTTGAATGAATACGAGCAAGCTTTTAC
AACAGTGCTGATCTAAAAATACTTAGCACTTGGCCTGAGATGCCTGGTGAGCATTACAGGCAAGGGGAATCTGGAGGT
AGCCGACCTGAGGACATGGCTTCTGAACCTGTCTTTTGGGAGTGGTATGGAAGGTGGAGCGTTCACCAGTGACCTGGA
AGGCCCAGCACCACCCTCCTTCCCACTCTTCTCATCTTGACAGAGCCTGCCCCAGCGCTGACGTGTCAGGAAAACACC
CAGGGAACTAGGAAGGCACTTCTGCCTGAGGGGCAGCCTGCCTTGCCCACTCCTGCTCTGCTCGCCTCGGATCAGCTG
AGCCTTCTGAGCTGGCCTCTCACTGCCTCCCCAAGGCCCCCTGCCTGCCCTGTCAGGAGGCAGAAGGAAGCAGGTGTG
AGGGCAGTGCAAGGAGGGAGCACAACCCCCAGCTCCCGCTCCGGGCTCCGACTTGTGCACAGGCAGAGCCCAGACCCT
GGAGGAAATCCTACCTTTGAATTCAAGAACATTTGGGGAATTTGGAAATCTCTTTGCCCCCAAACCCCCATTCTGTCC
TACCTTTAATCAGGTCCTGCTCAGCAGTGAGAGCAGATGAGGTGAAAAGGCCAAGAGGTTTGGCTCCTGCCCACTGAT
AGCCCCTCTCCCCGCAGTGTTTGTGTGTCAAGTGGCAAAGCTGTTCTTCCTGGTGACCCTGATTATATCCAGTAACAC
ATAGACTGTGCGCATAGGCCTGCTTTGTCTCCTCTATCCTGGGCTTTTGTTTTGCTTTTTAGTTTTGCTTTTAGTTTT
TCTGTCCCTTTTATTTAACGCACCGACTAGACACACAAAGCAGTTGAATTTTTATATATATATCTGTATATTGCACAA
TTATAAACTCATTTTGCTTGTGGCTCCACACACACAAAAAAAGACCTGTTAAAATTATACCTGTTGCTTAATTACAAT
ATTTCTGATAACCATAGCATAGGACAAGGGAAAATAAAAAAAGAAAAAAAAGAAAAAAAAACGACAAATCTGTCTGCT
GGTCACTTCTTCTGTCCAAGCAGATTCGTGGTCTTTTCCTCGCTTCTTTCAAGGGCTTTCCTGTGCCAGGTGAAGGAG
GCTCCAGGCAGCACCCAGGTTTTGCACTCTTGTTTCTCCCGTGCTTGTGAAAGAGGTCCCAAGGTTCTGGGTGCAGGA
GCGCTCCCTTGACCTGCTGAAGTCCGGAACGTAGTCGGCACAGCCTGGTCGCCTTCCACCTCTGGGAGCTGGAGTCCA
CTGGGGTGGCCTGACTCCCCCAGTCCCCTTCCCGTGACCTGGTCAGGGTGAGCCCATGTGGAGTCAGCCTCGCAGGCC
TCCCTGCCAGTAGGGTCCGAGTGTGTTTCATCCTTCCCACTCTGTCGAGCCTGGGGGCTGGAGCGGAGACGGGAGGCC
TGGCCTGTCTCGGAACCTGTGAGCTGCACCAGGTAGAACGCCAGGGACCCCAGAATCATGTGCGTCAGTCCAAGGGGT
CCCCTCCAGGAGTAGTGAAGACTCCAGAAATGTCCCTTTCTTCTCCCCCATCCTACGAGTAATTGCATTTGCTTTTGT
AATTCTTAATGAGCAATATCTGCTAGAGAGTTTAGCTGTAACAGTTCTTTTTGATCATCTTTTTTTAATAATTAGAAA
CACCAAAAAAATCCAGAAACTTGTTCTTCCAAAGCAGAGAGCATTATAATCACCAGGGCCAAAAGCTTCCCTCCCTGC
TGTCATTGCTTCTTCTGAGGCCTGAATCCAAAAGAAAAACAGCCATAGGCCCTTTCAGTGGCCGGGCTACCCGTGAGC
CCTTCGGAGGACCAGGGCTGGGGCAGCCTCTGGGCCCACATCCGGGGCCAGCTCCGGCGTGTGTTCAGTGTTAGCAGT
GGGTCATGATGCTCTTTCCCACCCAGCCTGGGATAGGGGCAGAGGAGGCGAGGAGGCCGTTGCCGCTGATGTTTGGCC
GTGAACAGGTGGGTGTCTGCGTGCGTCCACGTGCGTGTTTTCTGACTGACATGAAATCGACGCCCGAGTTAGCCTCAC
CCGGTGACCTCTAGCCCTGCCCGGATGGAGCGGGGCCCACCCGGTTCAGTGTTTCTGGGGAGCTGGACAGTGGAGTGC
AAAAGGCTTGCAGAACTTGAAGCCTGCTCCTTCCCTTGCTACCACGGCCTCCTTTCCGTTTGATTTGTCACTGCTTCA
ATCAATAACAGCCGCTCCAGAGTCAGTAGTCAATGAATATATGACCAAATATCACCAGGACTGTTACTCAATGTGTGC
CGAGCCCTTGCCCATGCTGGGCTCCCGTGTATCTGGACACTGTAACGTGTGCTGTGTTTGCTCCCCTTCCCCTTCCTT
CTTTGCCCTTTACTTGTCTTTCTGGGGTTTTTCTGTTTGGGTTTGGTTTGGTTTTTATTTCTCCTTTTGTGTTCCAAA
CATGAGGTTCTCTCTACTGGTCCTCTTAACTGTGGTGTTGAGGCTTATATTTGTGTAATTTTTGGTGGGTGAAAGGAA
TTTTGCTAAGTAAATCTCTTCTGTGTTTGAACTGAAGTCTGTATTGTAACTATGTTTAAAGTAATTGTTCCAGAGACA
AATATTTCTAGACACTTTTTCTTTACAAACAAAAGCATTCGGAGGGGGGGGATGGTGACTGAGATGAGAGGGGAGAG
CTGAACAGATGACCCCTGCCCAGATCAGCCAGAAGCCACCCAAAGCAGTGGAGCCCAGGAGTCCCACTCCAAGCCAGC
AAGCCGAATAGCTGATGTGTTGCCACTTTCCAAGTCACTGCAAAACCAGGTTTTGTTCCGCCCAGTGGATTCTTGTTT
TGCTTCCCCTCCCCCCGAGATTATTACCACCATCCCGTGCTTTTAAGGAAAGGCAAGATTGATGTTTCCTTGAGGGGA
GCCAGGAGGGGATGTGTGTGTGCAGAGCTGAAGAGCTGGGGAGAATGGGGCTGGGCCCACCCAAGCAGGAGGCTGGGA
CGCTCTGCTGTGGGCACAGGTCAGGCTAATGTTGGCAGATGCAGCTCTTCCTGGACAGGCCAGGTGGTGGGCATTCTC
TCTCCAAGGTGTGCCCCGTGGGCATTACTGTTTAAGACACTTCCGTCACATCCCACCCCATCCTCCAGGGCTCAACAC
TGTGACATCTCTATTCCCCACCCTCCCCTTCCCAGGGCAATAAAATGACCATGGAGGGGGCTTGCACTCTCTTGGCTG
TCACCCGATCGCCAGCAAAACTTAGATGTGAGAAAACCCCTTCCCATTCCATGGCGAAAACATCTCCTTAGAAAAGCC
ATTACCCTCATTAGGCATGGTTTTGGGCTCCCAAAACACCTGACAGCCCCTCCCTCCTCTGAGAGGCGGAGAGTGCTG
ACTGTAGTGACCATTGCATGCCGGGTGCAGCATCTGGAAGAGCTAGGCAGGGTGTCTGCCCCCTCCTGAGTTGAAGTC
ATGCTCCCCTGTGCCAGCCCAGAGGCCGAGAGCTATGGACAGCATTGCCAGTAACACAGGCCACCCTGTGCAGAAGGG
AGCTGGCTCCAGCCTGGAAACCTGTCTGAGGTTGGGAGAGGTGCACTTGGGGCACAGGGAGAGGCCGGGACACACTTA
GCTGGAGATGTCTCTAAAAGCCCTGTATCGTATTCACCTTCAGTTTTTGTGTTTTGGGACAATTACTTTAGAAAATAA
GTAGGTCGTTTTAAAAACAAAAATTATTGATTGCTTTTTTGTAGTGTTCAGAAAAAAGGTTCTTTGTGTATAGCCAAA
TGACTGAAAGCACTGATATATTTAAAAACAAAAGGCAATTTATTAAGGAAATTTGTACCATTTCAGTAAACCTGTCTG
AATGTACCTGTATACGTTTCAAAAACACCCCCCCCCCACTGAATCCCTGTAACCTATTTATTATATAAAGAGTTTGCC
TTATAAATTTACATAAAAATGTCCGTTTGTGTCTTTTGTTGTAAAAATCAAGTGATTTTTTCATAAGGTTCTTTTACT
ATTGGAAAAGATGGGCAGCACGCAGTTTTATTTTATTTTTGTAAGTTTTTTAATACATGTGAAAGCAAAGAATACTCA
GCATGCCTTTCTAAGTGACGCGTTTGCACCTTTTGTTGGGAAGTACTGTATCCTGTGCTGTTAGCATTCTCGATAAAT
CTCTCTGTGAAAGTGA
SEQ ID NO: 26 >NM_001110792.2 MECP2 [organism = Homo sapiens] [GeneID = 4204]
[transcript = 2]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGGAAGAAAAGTCAGAAGACCAGGACCTCCAGGGCCTCAAGGA
CAAACCCCTCAAGTTTAAAAAGGTGAAGAAAGATAAGAAAGAAGAGAAAGAGGGCAAGCATGAGCCCGTGCAGCCATC
AGCCCACCACTCTGCTGAGCCCGCAGAGGCAGGCAAAGCAGAGACATCAGAAGGGTCAGGCTCCGCCCCGGCTGTGCC
GGAAGCTTCTGCCTCCCCCAAACAGCGGCGCTCCATCATCCGTGACCGGGGACCCATGTATGATGACCCCACCCTGCC
TGAAGGCTGGACACGGAAGCTTAAGCAAAGGAAATCTGGCCGCTCTGCTGGGAAGTATGATGTGTATTTGATCAATCC
CCAGGGAAAAGCCTTTCGCTCTAAAGTGGAGTTGATTGCGTACTTCGAAAAGGTAGGCGACACATCCCTGGACCCTAA
TGATTTTGACTTCACGGTAACTGGGAGAGGGAGCCCCTCCCGGCGAGAGCAGAAACCACCTAAGAAGCCCAAATCTCC
CAAAGCTCCAGGAACTGGCAGAGGCCGGGGACGCCCCAAAGGGAGCGGCACCACGAGACCCAAGGCGGCCACGTCAGA
GGGTGTGCAGGTGAAAAGGGTCCTGGAGAAAAGTCCTGGGAAGCTCCTTGTCAAGATGCCTTTTCAAACTTCGCCAGG
GGGCAAGGCTGAGGGGGGTGGGGCCACCACATCCACCCAGGTCATGGTGATCAAACGCCCCGGCAGGAAGCGAAAAGC
TGAGGCCGACCCTCAGGCCATTCCCAAGAAACGGGGCCGAAAGCCGGGGAGTGTGGTGGCAGCCGCTGCCGCCGAGGC
CAAAAAGAAAGCCGTGAAGGAGTCTTCTATCCGATCTGTGCAGGAGACCGTACTCCCCATCAAGAAGCGCAAGACCCG
GGAGACGGTCAGCATCGAGGTCAAGGAAGTGGTGAAGCCCCTGCTGGTGTCCACCCTCGGTGAGAAGAGCGGGAAAGG
ACTGAAGACCTGTAAGAGCCCTGGGCGGAAAAGCAAGGAGAGCAGCCCCAAGGGGCGCAGCAGCAGCGCCTCCTCACC
CCCCAAGAAGGAGCACCACCACCATCACCACCACTCAGAGTCCCCAAAGGCCCCCGTGCCACTGCTCCCACCCCTGCC
CCCACCTCCACCTGAGCCCGAGAGCTCCGAGGACCCCACCAGCCCCCCTGAGCCCCAGGACTTGAGCAGCAGCGTCTG
CAAAGAGGAGAAGATGCCCAGAGGAGGCTCACTGGAGAGCGACGGCTGCCCCAAGGAGCCAGCTAAGACTCAGCCCGC
GGTTGCCACCGCCGCCACGGCCGCAGAAAAGTACAAACACCGAGGGGAGGGAGAGCGCAAAGACATTGTTTCATCCTC
CATGCCAAGGCCAAACAGAGAGGAGCCTGTGGACAGCCGGACGCCCGTGACCGAGAGAGTTAGCTGACTTTACACGGA
GCGGATTGCAAAGCAAACCAACAAGAATAAAGGCAGCTGTTGTCTCTTCTCCTTATGGGTAGGGCTCTGACAAAGCTT
CCCGATTAACTGAAATAAAAAATATTTTTTTTTCTTTCAGTAAACTTAGAGTTTCGTGGCTTCAGGGTGGGAGTAGTT
GGAGCATTGGGGATGTTTTTCTTACCGACAAGCACAGTCAGGTTGAAGACCTAACCAGGGCCAGAAGTAGCTTTGCAC
TTTTCTAAACTAGGCTCCTTCAACAAGGCTTGCTGCAGATACTACTGACCAGACAAGCTGTTGACCAGGCACCTCCCC
TCCCGCCCAAACCTTTCCCCCATGTGGTCGTTAGAGACAGAGCGACAGAGCAGTTGAGAGGACACTCCCGTTTTCGGT
GCCATCAGTGCCCCGTCTACAGCTCCCCCAGCTCCCCCCACCTCCCCCACTCCCAACCACGTTGGGACAGGGAGGTGT
GAGGCAGGAGAGACAGTTGGATTCTTTAGAGAAGATGGATATGACCAGTGGCTATGGCCTGTGCGATCCCACCCGTGG
TGGCTCAAGTCTGGCCCCACACCAGCCCCAATCCAAAACTGGCAAGGACGCTTCACAGGACAGGAAAGTGGCACCTGT
CTGCTCCAGCTCTGGCATGGCTAGGAGGGGGGAGTCCCTTGAACTACTGGGTGTAGACTGGCCTGAACCACAGGAGAG
GATGGCCCAGGGTGAGGTGGCATGGTCCATTCTCAAGGGACGTCCTCCAACGGGTGGCGCTAGAGGCCATGGAGGCAG
TAGGACAAGGTGCAGGCAGGCTGGCCTGGGGTCAGGCCGGGCAGAGCACAGCGGGGTGAGAGGGATTCCTAATCACTC
AGAGCAGTCTGTGACTTAGTGGACAGGGGAGGGGGCAAAGGGGGAGGAGAAGAAAATGTTCTTCCAGTTACTTTCCAA
TTCTCCTTTAGGGACAGCTTAGAATTATTTGCACTATTGAGTCTTCATGTTCCCACTTCAAAACAAACAGATGCTCTG
AGAGCAAACTGGCTTGAATTGGTGACATTTAGTCCCTCAAGCCACCAGATGTGACAGTGTTGAGAACTACCTGGATTT
GTATATATACCTGCGCTTGTTTTAAAGTGGGCTCAGCACATAGGGTTCCCACGAAGCTCCGAAACTCTAAGTGTTTGC
TGCAATTTTATAAGGACTTCCTGATTGGTTTCTCTTCTCCCCTTCCATTTCTGCCTTTTGTTCATTTCATCCTTTCAC
TTCTTTCCCTTCCTCCATCCTCCTCCTTCCTAGTTCATCCCTTCTCTTCCAGGCAGCCGCGGTGCCCAACCACACTTG
TCGGCTCCAGTCCCCAGAACTCTGCCTGCCCTTTGTCCTCCTGCTGCCAGTACCAGCCCCACCCTGTTTTGAGCCCTG
AGGAGGCCTTGGGCTCTGCTGAGTCCGACCTGGCCTGTCTGTGAAGAGCAAGAGAGCAGCAAGGTCTTGCTCTCCTAG
GTAGCCCCCTCTTCCCTGGTAAGAAAAAGCAAAAGGCATTTCCCACCCTGAACAACGAGCCTTTTCACCCTTCTACTC
TAGAGAAGTGGACTGGAGGAGCTGGGCCCGATTTGGTAGTTGAGGAAAGCACAGAGGCCTCCTGTGGCCTGCCAGTCA
TCGAGTGGCCCAACAGGGGCTCCATGCCAGCCGACCTTGACCTCACTCAGAAGTCCAGAGTCTAGCGTAGTGCAGCAG
GGCAGTAGCGGTACCAATGCAGAACTCCCAAGACCCGAGCTGGGACCAGTACCTGGGTCCCCAGCCCTTCCTCTGCTC
CCCCTTTTCCCTCGGAGTTCTTCTTGAATGGCAATGTTTTGCTTTTGCTCGATGCAGACAGGGGGCCAGAACACCACA
CATTTCACTGTCTGTCTGGTCCATAGCTGTGGTGTAGGGGCTTAGAGGCATGGGCTTGCTGTGGGTTTTTAATTGATC
AGTTTTCATGTGGGATCCCATCTTTTTAACCTCTGTTCAGGAAGTCCTTATCTAGCTGCATATCTTCATCATATTGGT
ATATCCTTTTCTGTGTTTACAGAGATGTCTCTTATATCTAAATCTGTCCAACTGAGAAGTACCTTATCAAAGTAGCAA
ATGAGACAGCAGTCTTATGCTTCCAGAAACACCCACAGGCATGTCCCATGTGAGCTGCTGCCATGAACTGTCAAGTGT
GTGTTGTCTTGTGTATTTCAGTTATTGTCCCTGGCTTCCTTACTATGGTGTAATCATGAAGGAGTGAAACATCATAGA
AACTGTCTAGCACTTCCTTGCCAGTCTTTAGTGATCAGGAACCATAGTTGACAGTTCCAATCAGTAGCTTAAGAAAAA
ACCGTGTTTGTCTCTTCTGGAATGGTTAGAAGTGAGGGAGTTTGCCCCGTTCTGTTTGTAGAGTCTCATAGTTGGACT
TTCTAGCATATATGTGTCCATTTCCTTATGCTGTAAAAGCAAGTCCTGCAACCAAACTCCCATCAGCCCAATCCCTGA
TCCCTGATCCCTTCCACCTGCTCTGCTGATGACCCCCCCAGCTTCACTTCTGACTCTTCCCCAGGAAGGGAAGGGGGG
TCAGAAGAGAGGGTGAGTCCTCCAGAACTCTTCCTCCAAGGACAGAAGGCTCCTGCCCCCATAGTGGCCTCGAACTCC
TGGCACTACCAAAGGACACTTATCCACGAGAGCGCAGCATCCGACCAGGTTGTCACTGAGAAGATGTTTATTTTGGTC
AGTTGGGTTTTTATGTATTATACTTAGTCAAATGTAATGTGGCTTCTGGAATCATTGTCCAGAGCTGCTTCCCCGTCA
CCTGGGCGTCATCTGGTCCTGGTAAGAGGAGTGCGTGGCCCACCAGGCCCCCCTGTCACCCATGACAGTTCATTCAGG
GCCGATGGGGCAGTCGTGGTTGGGAACACAGCATTTCAAGCGTCACTTTATTTCATTCGGGCCCCACCTGCAGCTCCC
TCAAAGAGGCAGTTGCCCAGCCTCTTTCCCTTCCAGTTTATTCCAGAGCTGCCAGTGGGGCCTGAGGCTCCTTAGGGT
TTTCTCTCTATTTCCCCCTTTCTTCCTCATTCCCTCGTCTTTCCCAAAGGCATCACGAGTCAGTCGCCTTTCAGCAGG
CAGCCTTGGCGGTTTATCGCCCTGGCAGGCAGGGGCCCTGCAGCTCTCATGCTGCCCCTGCCTTGGGGTCAGGTTGAC
AGGAGGTTGGAGGGAAAGCCTTAAGCTGCAGGATTCTCACCAGCTGTGTCCGGCCCAGTTTTGGGGTGTGACCTCAAT
TTCAATTTTGTCTGTACTTGAACATTATGAAGATGGGGGCCTCTTTCAGTGAATTTGTGAACAGCAGAATTGACCGAC
AGCTTTCCAGTACCCATGGGGCTAGGTCATTAAGGCCACATCCACAGTCTCCCCCACCCTTGTTCCAGTTGTTAGTTA
CTACCTCCTCTCCTGACAATACTGTATGTCGTCGAGCTCCCCCCAGGTCTACCCCTCCCGGCCCTGCCTGCTGGTGGG
CTTGTCATAGCCAGTGGGATTGCCGGTCTTGACAGCTCAGTGAGCTGGAGATACTTGGTCACAGCCAGGCGCTAGCAC
AGCTCCCTTCTGTTGATGCTGTATTCCCATATCAAAAGACACAGGGGACACCCAGAAACGCCACATCCCCCAATCCAT
CAGTGCCAAACTAGCCAACGGCCCCAGCTTCTCAGCTCGCTGGATGGCGGAAGCTGCTACTCGTGAGCGCCAGTGCGG
GTGCAGACAATCTTCTGTTGGGTGGCATCATTCCAGGCCCGAAGCATGAACAGTGCACCTGGGACAGGGAGCAGCCCC
AAATTGTCACCTGCTTCTCTGCCCAGCTTTTCATTGCTGTGACAGTGATGGCGAAAGAGGGTAATAACCAGACACAAA
CTGCCAAGTTGGGTGGAGAAAGGAGTTTCTTTAGCTGACAGAATCTCTGAATTTTAAATCACTTAGTAAGCGGCTCAA
GCCCAGGAGGGAGCAGAGGGATACGAGCGGAGTCCCCTGCGCGGGACCATCTGGAATTGGTTTAGCCCAAGTGGAGCC
TGACAGCCAGAACTCTGTGTCCCCCGTCTAACCACAGCTCCTTTTCCAGAGCATTCCAGTCAGGCTCTCTGGGCTGAC
TGGGCCAGGGGAGGTTACAGGTACCAGTTCTTTAAGAAGATCTTTGGGCATATACATTTTTAGCCTGTGTCATTGCCC
CAAATGGATTCCTGTTTCAAGTTCACACCTGCAGATTCTAGGACCTGTGTCCTAGACTTCAGGGAGTCAGCTGTTTCT
AGAGTTCCTACCATGGAGTGGGTCTGGAGGACCTGCCCGGTGGGGGGGCAGAGCCCTGCTCCCTCCGGGTCTTCCTAC
TCTTCTCTCTGCTCTGACGGGATTTGTTGATTCTCTCCATTTTGGTGTCTTTCTCTTTTAGATATTGTATCAATCTTT
AGAAAAGGCATAGTCTACTTGTTATAAATCGTTAGGATACTGCCTCCCCCAGGGTCTAAAATTACATATTAGAGGGGA
AAAGCTGAACACTGAAGTCAGTTCTCAACAATTTAGAAGGAAAACCTAGAAAACATTTGGCAGAAAATTACATTTCGA
TGTTTTTGAATGAATACGAGCAAGCTTTTACAACAGTGCTGATCTAAAAATACTTAGCACTTGGCCTGAGATGCCTGG
TGAGCATTACAGGCAAGGGGAATCTGGAGGTAGCCGACCTGAGGACATGGCTTCTGAACCTGTCTTTTGGGAGTGGTA
TGGAAGGTGGAGCGTTCACCAGTGACCTGGAAGGCCCAGCACCACCCTCCTTCCCACTCTTCTCATCTTGACAGAGCC
TGCCCCAGCGCTGACGTGTCAGGAAAACACCCAGGGAACTAGGAAGGCACTTCTGCCTGAGGGGCAGCCTGCCTTGCC
CACTCCTGCTCTGCTCGCCTCGGATCAGCTGAGCCTTCTGAGCTGGCCTCTCACTGCCTCCCCAAGGCCCCCTGCCTG
CCCTGTCAGGAGGCAGAAGGAAGCAGGTGTGAGGGCAGTGCAAGGAGGGAGCACAACCCCCAGCTCCCGCTCCGGGCT
CCGACTTGTGCACAGGCAGAGCCCAGACCCTGGAGGAAATCCTACCTTTGAATTCAAGAACATTTGGGGAATTTGGAA
ATCTCTTTGCCCCCAAACCCCCATTCTGTCCTACCTTTAATCAGGTCCTGCTCAGCAGTGAGAGCAGATGAGGTGAAA
AGGCCAAGAGGTTTGGCTCCTGCCCACTGATAGCCCCTCTCCCCGCAGTGTTTGTGTGTCAAGTGGCAAAGCTGTTCT
TCCTGGTGACCCTGATTATATCCAGTAACACATAGACTGTGCGCATAGGCCTGCTTTGTCTCCTCTATCCTGGGCTTT
TGTTTTGCTTTTTAGTTTTGCTTTTAGTTTTTCTGTCCCTTTTATTTAACGCACCGACTAGACACACAAAGCAGTTGA
ATTTTTATATATATATCTGTATATTGCACAATTATAAACTCATTTTGCTTGTGGCTCCACACACACAAAAAAAGACCT
GTTAAAATTATACCTGTTGCTTAATTACAATATTTCTGATAACCATAGCATAGGACAAGGGAAAATAAAAAAAGAAAA
AAAAGAAAAAAAAACGACAAATCTGTCTGCTGGTCACTTCTTCTGTCCAAGCAGATTCGTGGTCTTTTCCTCGCTTCT
TTCAAGGGCTTTCCTGTGCCAGGTGAAGGAGGCTCCAGGCAGCACCCAGGTTTTGCACTCTTGTTTCTCCCGTGCTTG
TGAAAGAGGTCCCAAGGTTCTGGGTGCAGGAGCGCTCCCTTGACCTGCTGAAGTCCGGAACGTAGTCGGCACAGCCTG
GTCGCCTTCCACCTCTGGGAGCTGGAGTCCACTGGGGTGGCCTGACTCCCCCAGTCCCCTTCCCGTGACCTGGTCAGG
GTGAGCCCATGTGGAGTCAGCCTCGCAGGCCTCCCTGCCAGTAGGGTCCGAGTGTGTTTCATCCTTCCCACTCTGTCG
AGCCTGGGGGCTGGAGCGGAGACGGGAGGCCTGGCCTGTCTCGGAACCTGTGAGCTGCACCAGGTAGAACGCCAGGGA
CCCCAGAATCATGTGCGTCAGTCCAAGGGGTCCCCTCCAGGAGTAGTGAAGACTCCAGAAATGTCCCTTTCTTCTCCC
CCATCCTACGAGTAATTGCATTTGCTTTTGTAATTCTTAATGAGCAATATCTGCTAGAGAGTTTAGCTGTAACAGTTC
TTTTTGATCATCTTTTTTTAATAATTAGAAACACCAAAAAAATCCAGAAACTTGTTCTTCCAAAGCAGAGAGCATTAT
AATCACCAGGGCCAAAAGCTTCCCTCCCTGCTGTCATTGCTTCTTCTGAGGCCTGAATCCAAAAGAAAAACAGCCATA
GGCCCTTTCAGTGGCCGGGCTACCCGTGAGCCCTTCGGAGGACCAGGGCTGGGGCAGCCTCTGGGCCCACATCCGGGG
CCAGCTCCGGCGTGTGTTCAGTGTTAGCAGTGGGTCATGATGCTCTTTCCCACCCAGCCTGGGATAGGGGCAGAGGAG
GCGAGGAGGCCGTTGCCGCTGATGTTTGGCCGTGAACAGGTGGGTGTCTGCGTGCGTCCACGTGCGTGTTTTCTGACT
GACATGAAATCGACGCCCGAGTTAGCCTCACCCGGTGACCTCTAGCCCTGCCCGGATGGAGCGGGGCCCACCCGGTTC
AGTGTTTCTGGGGAGCTGGACAGTGGAGTGCAAAAGGCTTGCAGAACTTGAAGCCTGCTCCTTCCCTTGCTACCACGG
CCTCCTTTCCGTTTGATTTGTCACTGCTTCAATCAATAACAGCCGCTCCAGAGTCAGTAGTCAATGAATATATGACCA
AATATCACCAGGACTGTTACTCAATGTGTGCCGAGCCCTTGCCCATGCTGGGCTCCCGTGTATCTGGACACTGTAACG
TGTGCTGTGTTTGCTCCCCTTCCCCTTCCTTCTTTGCCCTTTACTTGTCTTTCTGGGGTTTTTCTGTTTGGGTTTGGT
TTGGTTTTTATTTCTCCTTTTGTGTTCCAAACATGAGGTTCTCTCTACTGGTCCTCTTAACTGTGGTGTTGAGGCTTA
TATTTGTGTAATTTTTGGTGGGTGAAAGGAATTTTGCTAAGTAAATCTCTTCTGTGTTTGAACTGAAGTCTGTATTGT
AACTATGTTTAAAGTAATTGTTCCAGAGACAAATATTTCTAGACACTTTTTCTTTACAAACAAAAGCATTCGGAGGGA
GGGGGATGGTGACTGAGATGAGAGGGGAGAGCTGAACAGATGACCCCTGCCCAGATCAGCCAGAAGCCACCCAAAGCA
GTGGAGCCCAGGAGTCCCACTCCAAGCCAGCAAGCCGAATAGCTGATGTGTTGCCACTTTCCAAGTCACTGCAAAACC
AGGTTTTGTTCCGCCCAGTGGATTCTTGTTTTGCTTCCCCTCCCCCCGAGATTATTACCACCATCCCGTGCTTTTAAG
GAAAGGCAAGATTGATGTTTCCTTGAGGGGAGCCAGGAGGGGATGTGTGTGTGCAGAGCTGAAGAGCTGGGGAGAATG
GGGCTGGGCCCACCCAAGCAGGAGGCTGGGACGCTCTGCTGTGGGCACAGGTCAGGCTAATGTTGGCAGATGCAGCTC
TTCCTGGACAGGCCAGGTGGTGGGCATTCTCTCTCCAAGGTGTGCCCCGTGGGCATTACTGTTTAAGACACTTCCGTC
ACATCCCACCCCATCCTCCAGGGCTCAACACTGTGACATCTCTATTCCCCACCCTCCCCTTCCCAGGGCAATAAAATG
ACCATGGAGGGGGCTTGCACTCTCTTGGCTGTCACCCGATCGCCAGCAAAACTTAGATGTGAGAAAACCCCTTCCCAT
TCCATGGCGAAAACATCTCCTTAGAAAAGCCATTACCCTCATTAGGCATGGTTTTGGGCTCCCAAAACACCTGACAGC
CCCTCCCTCCTCTGAGAGGCGGAGAGTGCTGACTGTAGTGACCATTGCATGCCGGGTGCAGCATCTGGAAGAGCTAGG
CAGGGTGTCTGCCCCCTCCTGAGTTGAAGTCATGCTCCCCTGTGCCAGCCCAGAGGCCGAGAGCTATGGACAGCATTG
CCAGTAACACAGGCCACCCTGTGCAGAAGGGAGCTGGCTCCAGCCTGGAAACCTGTCTGAGGTTGGGAGAGGTGCACT
TGGGGCACAGGGAGAGGCCGGGACACACTTAGCTGGAGATGTCTCTAAAAGCCCTGTATCGTATTCACCTTCAGTTTT
TGTGTTTTGGGACAATTACTTTAGAAAATAAGTAGGTCGTTTTAAAAACAAAAATTATTGATTGCTTTTTTGTAGTGT
TCAGAAAAAAGGTTCTTTGTGTATAGCCAAATGACTGAAAGCACTGATATATTTAAAAACAAAAGGCAATTTATTAAG
GAAATTTGTACCATTTCAGTAAACCTGTCTGAATGTACCTGTATACGTTTCAAAAACACCCCCCCCCCACTGAATCCC
TGTAACCTATTTATTATATAAAGAGTTTGCCTTATAAATTTACATAAAAATGTCCGTTTGTGTCTTTTGTTGTAAAAA
TCAAGTGATTTTTTCATAAGGTTCTTTTACTATTGGAAAAGATGGGCAGCACGCAGTTTTATTTTATTTTTGTAAGTT
TTTTAATACATGTGAAAGCAAAGAATACTCAGCATGCCTTTCTAAGTGACGCGTTTGCACCTTTTGTTGGGAAGTACT
GTATCCTGTGCTGTTAGCATTCTCGATAAATCTCTCTGTGAAAGTGA
SEQ ID NO: 27 >NM_001369393.2 MECP2 [organism = Homo sapiens] [GeneID = 4204]
[transcript = 6]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTCACTGAGCTAGCCTCTCCTTGCTAGAAGCTTACGGTCGAAGG
TCTCCAGCCATCAGAAGAAGCACGTGGAGCGCTGCGTCCGTGTTGTGGTTATTCATCCAGCATGTGTTGAGGAAGAAA
AGTCAGAAGACCAGGACCTCCAGGGCCTCAAGGACAAACCCCTCAAGTTTAAAAAGGTGAAGAAAGATAAGAAAGAAG
AGAAAGAGGGCAAGCATGAGCCCGTGCAGCCATCAGCCCACCACTCTGCTGAGCCCGCAGAGGCAGGCAAAGCAGAGA
CATCAGAAGGGTCAGGCTCCGCCCCGGCTGTGCCGGAAGCTTCTGCCTCCCCCAAACAGCGGCGCTCCATCATCCGTG
ACCGGGGACCCATGTATGATGACCCCACCCTGCCTGAAGGCTGGACACGGAAGCTTAAGCAAAGGAAATCTGGCCGCT
CTGCTGGGAAGTATGATGTGTATTTGATCAATCCCCAGGGAAAAGCCTTTCGCTCTAAAGTGGAGTTGATTGCGTACT
TCGAAAAGGTAGGCGACACATCCCTGGACCCTAATGATTTTGACTTCACGGTAACTGGGAGAGGGAGCCCCTCCCGGC
GAGAGCAGAAACCACCTAAGAAGCCCAAATCTCCCAAAGCTCCAGGAACTGGCAGAGGCCGGGGACGCCCCAAAGGGA
GCGGCACCACGAGACCCAAGGCGGCCACGTCAGAGGGTGTGCAGGTGAAAAGGGTCCTGGAGAAAAGTCCTGGGAAGC
TCCTTGTCAAGATGCCTTTTCAAACTTCGCCAGGGGGCAAGGCTGAGGGGGGTGGGGCCACCACATCCACCCAGGTCA
TGGTGATCAAACGCCCCGGCAGGAAGCGAAAAGCTGAGGCCGACCCTCAGGCCATTCCCAAGAAACGGGGCCGAAAGC
CGGGGAGTGTGGTGGCAGCCGCTGCCGCCGAGGCCAAAAAGAAAGCCGTGAAGGAGTCTTCTATCCGATCTGTGCAGG
AGACCGTACTCCCCATCAAGAAGCGCAAGACCCGGGAGACGGTCAGCATCGAGGTCAAGGAAGTGGTGAAGCCCCTGC
TGGTGTCCACCCTCGGTGAGAAGAGCGGGAAAGGACTGAAGACCTGTAAGAGCCCTGGGCGGAAAAGCAAGGAGAGCA
GCCCCAAGGGGCGCAGCAGCAGCGCCTCCTCACCCCCCAAGAAGGAGCACCACCACCATCACCACCACTCAGAGTCCC
CAAAGGCCCCCGTGCCACTGCTCCCACCCCTGCCCCCACCTCCACCTGAGCCCGAGAGCTCCGAGGACCCCACCAGCC
CCCCTGAGCCCCAGGACTTGAGCAGCAGCGTCTGCAAAGAGGAGAAGATGCCCAGAGGAGGCTCACTGGAGAGCGACG
GCTGCCCCAAGGAGCCAGCTAAGACTCAGCCCGCGGTTGCCACCGCCGCCACGGCCGCAGAAAAGTACAAACACCGAG
GGGAGGGAGAGCGCAAAGACATTGTTTCATCCTCCATGCCAAGGCCAAACAGAGAGGAGCCTGTGGACAGCCGGACGC
CCGTGACCGAGAGAGTTAGCTGACTTTACACGGAGCGGATTGCAAAGCAAACCAACAAGAATAAAGGCAGCTGTTGTC
TCTTCTCCTTATGGGTAGGGCTCTGACAAAGCTTCCCGATTAACTGAAATAAAAAATATTTTTTTTTCTTTCAGTAAA
CTTAGAGTTTCGTGGCTTCAGGGTGGGAGTAGTTGGAGCATTGGGGATGTTTTTCTTACCGACAAGCACAGTCAGGTT
GAAGACCTAACCAGGGCCAGAAGTAGCTTTGCACTTTTCTAAACTAGGCTCCTTCAACAAGGCTTGCTGCAGATACTA
CTGACCAGACAAGCTGTTGACCAGGCACCTCCCCTCCCGCCCAAACCTTTCCCCCATGTGGTCGTTAGAGACAGAGCG
ACAGAGCAGTTGAGAGGACACTCCCGTTTTCGGTGCCATCAGTGCCCCGTCTACAGCTCCCCCAGCTCCCCCCACCTC
CCCCACTCCCAACCACGTTGGGACAGGGAGGTGTGAGGCAGGAGAGACAGTTGGATTCTTTAGAGAAGATGGATATGA
CCAGTGGCTATGGCCTGTGCGATCCCACCCGTGGTGGCTCAAGTCTGGCCCCACACCAGCCCCAATCCAAAACTGGCA
AGGACGCTTCACAGGACAGGAAAGTGGCACCTGTCTGCTCCAGCTCTGGCATGGCTAGGAGGGGGGAGTCCCTTGAAC
TACTGGGTGTAGACTGGCCTGAACCACAGGAGAGGATGGCCCAGGGTGAGGTGGCATGGTCCATTCTCAAGGGACGTC
CTCCAACGGGTGGCGCTAGAGGCCATGGAGGCAGTAGGACAAGGTGCAGGCAGGCTGGCCTGGGGTCAGGCCGGGCAG
AGCACAGCGGGGTGAGAGGGATTCCTAATCACTCAGAGCAGTCTGTGACTTAGTGGACAGGGGAGGGGGCAAAGGGGG
AGGAGAAGAAAATGTTCTTCCAGTTACTTTCCAATTCTCCTTTAGGGACAGCTTAGAATTATTTGCACTATTGAGTCT
TCATGTTCCCACTTCAAAACAAACAGATGCTCTGAGAGCAAACTGGCTTGAATTGGTGACATTTAGTCCCTCAAGCCA
CCAGATGTGACAGTGTTGAGAACTACCTGGATTTGTATATATACCTGCGCTTGTTTTAAAGTGGGCTCAGCACATAGG
GTTCCCACGAAGCTCCGAAACTCTAAGTGTTTGCTGCAATTTTATAAGGACTTCCTGATTGGTTTCTCTTCTCCCCTT
CCATTTCTGCCTTTTGTTCATTTCATCCTTTCACTTCTTTCCCTTCCTCCATCCTCCTCCTTCCTAGTTCATCCCTTC
TCTTCCAGGCAGCCGCGGTGCCCAACCACACTTGTCGGCTCCAGTCCCCAGAACTCTGCCTGCCCTTTGTCCTCCTGC
TGCCAGTACCAGCCCCACCCTGTTTTGAGCCCTGAGGAGGCCTTGGGCTCTGCTGAGTCCGACCTGGCCTGTCTGTGA
AGAGCAAGAGAGCAGCAAGGTCTTGCTCTCCTAGGTAGCCCCCTCTTCCCTGGTAAGAAAAAGCAAAAGGCATTTCCC
ACCCTGAACAACGAGCCTTTTCACCCTTCTACTCTAGAGAAGTGGACTGGAGGAGCTGGGCCCGATTTGGTAGTTGAG
GAAAGCACAGAGGCCTCCTGTGGCCTGCCAGTCATCGAGTGGCCCAACAGGGGCTCCATGCCAGCCGACCTTGACCTC
ACTCAGAAGTCCAGAGTCTAGCGTAGTGCAGCAGGGCAGTAGCGGTACCAATGCAGAACTCCCAAGACCCGAGCTGGG
ACCAGTACCTGGGTCCCCAGCCCTTCCTCTGCTCCCCCTTTTCCCTCGGAGTTCTTCTTGAATGGCAATGTTTTGCTT
TTGCTCGATGCAGACAGGGGGCCAGAACACCACACATTTCACTGTCTGTCTGGTCCATAGCTGTGGTGTAGGGGCTTA
GAGGCATGGGCTTGCTGTGGGTTTTTAATTGATCAGTTTTCATGTGGGATCCCATCTTTTTAACCTCTGTTCAGGAAG
TCCTTATCTAGCTGCATATCTTCATCATATTGGTATATCCTTTTCTGTGTTTACAGAGATGTCTCTTATATCTAAATC
TGTCCAACTGAGAAGTACCTTATCAAAGTAGCAAATGAGACAGCAGTCTTATGCTTCCAGAAACACCCACAGGCATGT
CCCATGTGAGCTGCTGCCATGAACTGTCAAGTGTGTGTTGTCTTGTGTATTTCAGTTATTGTCCCTGGCTTCCTTACT
ATGGTGTAATCATGAAGGAGTGAAACATCATAGAAACTGTCTAGCACTTCCTTGCCAGTCTTTAGTGATCAGGAACCA
TAGTTGACAGTTCCAATCAGTAGCTTAAGAAAAAACCGTGTTTGTCTCTTCTGGAATGGTTAGAAGTGAGGGAGTTTG
CCCCGTTCTGTTTGTAGAGTCTCATAGTTGGACTTTCTAGCATATATGTGTCCATTTCCTTATGCTGTAAAAGCAAGT
CCTGCAACCAAACTCCCATCAGCCCAATCCCTGATCCCTGATCCCTTCCACCTGCTCTGCTGATGACCCCCCCAGCTT
CACTTCTGACTCTTCCCCAGGAAGGGAAGGGGGGTCAGAAGAGAGGGTGAGTCCTCCAGAACTCTTCCTCCAAGGACA
GAAGGCTCCTGCCCCCATAGTGGCCTCGAACTCCTGGCACTACCAAAGGACACTTATCCACGAGAGCGCAGCATCCGA
CCAGGTTGTCACTGAGAAGATGTTTATTTTGGTCAGTTGGGTTTTTATGTATTATACTTAGTCAAATGTAATGTGGCT
TCTGGAATCATTGTCCAGAGCTGCTTCCCCGTCACCTGGGCGTCATCTGGTCCTGGTAAGAGGAGTGCGTGGCCCACC
AGGCCCCCCTGTCACCCATGACAGTTCATTCAGGGCCGATGGGGCAGTCGTGGTTGGGAACACAGCATTTCAAGCGTC
ACTTTATTTCATTCGGGCCCCACCTGCAGCTCCCTCAAAGAGGCAGTTGCCCAGCCTCTTTCCCTTCCAGTTTATTCC
AGAGCTGCCAGTGGGGCCTGAGGCTCCTTAGGGTTTTCTCTCTATTTCCCCCTTTCTTCCTCATTCCCTCGTCTTTCC
CAAAGGCATCACGAGTCAGTCGCCTTTCAGCAGGCAGCCTTGGCGGTTTATCGCCCTGGCAGGCAGGGGCCCTGCAGC
TCTCATGCTGCCCCTGCCTTGGGGTCAGGTTGACAGGAGGTTGGAGGGAAAGCCTTAAGCTGCAGGATTCTCACCAGC
TGTGTCCGGCCCAGTTTTGGGGTGTGACCTCAATTTCAATTTTGTCTGTACTTGAACATTATGAAGATGGGGGCCTCT
TTCAGTGAATTTGTGAACAGCAGAATTGACCGACAGCTTTCCAGTACCCATGGGGCTAGGTCATTAAGGCCACATCCA
CAGTCTCCCCCACCCTTGTTCCAGTTGTTAGTTACTACCTCCTCTCCTGACAATACTGTATGTCGTCGAGCTCCCCCC
AGGTCTACCCCTCCCGGCCCTGCCTGCTGGTGGGCTTGTCATAGCCAGTGGGATTGCCGGTCTTGACAGCTCAGTGAG
CTGGAGATACTTGGTCACAGCCAGGCGCTAGCACAGCTCCCTTCTGTTGATGCTGTATTCCCATATCAAAAGACACAG
GGGACACCCAGAAACGCCACATCCCCCAATCCATCAGTGCCAAACTAGCCAACGGCCCCAGCTTCTCAGCTCGCTGGA
TGGCGGAAGCTGCTACTCGTGAGCGCCAGTGCGGGTGCAGACAATCTTCTGTTGGGTGGCATCATTCCAGGCCCGAAG
CATGAACAGTGCACCTGGGACAGGGAGCAGCCCCAAATTGTCACCTGCTTCTCTGCCCAGCTTTTCATTGCTGTGACA
GTGATGGCGAAAGAGGGTAATAACCAGACACAAACTGCCAAGTTGGGTGGAGAAAGGAGTTTCTTTAGCTGACAGAAT
CTCTGAATTTTAAATCACTTAGTAAGCGGCTCAAGCCCAGGAGGGAGCAGAGGGATACGAGCGGAGTCCCCTGCGCGG
GACCATCTGGAATTGGTTTAGCCCAAGTGGAGCCTGACAGCCAGAACTCTGTGTCCCCCGTCTAACCACAGCTCCTTT
TCCAGAGCATTCCAGTCAGGCTCTCTGGGCTGACTGGGCCAGGGGAGGTTACAGGTACCAGTTCTTTAAGAAGATCTT
TGGGCATATACATTTTTAGCCTGTGTCATTGCCCCAAATGGATTCCTGTTTCAAGTTCACACCTGCAGATTCTAGGAC
CTGTGTCCTAGACTTCAGGGAGTCAGCTGTTTCTAGAGTTCCTACCATGGAGTGGGTCTGGAGGACCTGCCCGGTGGG
GGGGCAGAGCCCTGCTCCCTCCGGGTCTTCCTACTCTTCTCTCTGCTCTGACGGGATTTGTTGATTCTCTCCATTTTG
GTGTCTTTCTCTTTTAGATATTGTATCAATCTTTAGAAAAGGCATAGTCTACTTGTTATAAATCGTTAGGATACTGCC
TCCCCCAGGGTCTAAAATTACATATTAGAGGGGAAAAGCTGAACACTGAAGTCAGTTCTCAACAATTTAGAAGGAAAA
CCTAGAAAACATTTGGCAGAAAATTACATTTCGATGTTTTTGAATGAATACGAGCAAGCTTTTACAACAGTGCTGATC
TAAAAATACTTAGCACTTGGCCTGAGATGCCTGGTGAGCATTACAGGCAAGGGGAATCTGGAGGTAGCCGACCTGAGG
ACATGGCTTCTGAACCTGTCTTTTGGGAGTGGTATGGAAGGTGGAGCGTTCACCAGTGACCTGGAAGGCCCAGCACCA
CCCTCCTTCCCACTCTTCTCATCTTGACAGAGCCTGCCCCAGCGCTGACGTGTCAGGAAAACACCCAGGGAACTAGGA
AGGCACTTCTGCCTGAGGGGCAGCCTGCCTTGCCCACTCCTGCTCTGCTCGCCTCGGATCAGCTGAGCCTTCTGAGCT
GGCCTCTCACTGCCTCCCCAAGGCCCCCTGCCTGCCCTGTCAGGAGGCAGAAGGAAGCAGGTGTGAGGGCAGTGCAAG
GAGGGAGCACAACCCCCAGCTCCCGCTCCGGGCTCCGACTTGTGCACAGGCAGAGCCCAGACCCTGGAGGAAATCCTA
CCTTTGAATTCAAGAACATTTGGGGAATTTGGAAATCTCTTTGCCCCCAAACCCCCATTCTGTCCTACCTTTAATCAG
GTCCTGCTCAGCAGTGAGAGCAGATGAGGTGAAAAGGCCAAGAGGTTTGGCTCCTGCCCACTGATAGCCCCTCTCCCC
GCAGTGTTTGTGTGTCAAGTGGCAAAGCTGTTCTTCCTGGTGACCCTGATTATATCCAGTAACACATAGACTGTGCGC
ATAGGCCTGCTTTGTCTCCTCTATCCTGGGCTTTTGTTTTGCTTTTTAGTTTTGCTTTTAGTTTTTCTGTCCCTTTTA
TTTAACGCACCGACTAGACACACAAAGCAGTTGAATTTTTATATATATATCTGTATATTGCACAATTATAAACTCATT
TTGCTTGTGGCTCCACACACACAAAAAAAGACCTGTTAAAATTATACCTGTTGCTTAATTACAATATTTCTGATAACC
ATAGCATAGGACAAGGGAAAATAAAAAAAGAAAAAAAAGAAAAAAAAACGACAAATCTGTCTGCTGGTCACTTCTTCT
GTCCAAGCAGATTCGTGGTCTTTTCCTCGCTTCTTTCAAGGGCTTTCCTGTGCCAGGTGAAGGAGGCTCCAGGCAGCA
CCCAGGTTTTGCACTCTTGTTTCTCCCGTGCTTGTGAAAGAGGTCCCAAGGTTCTGGGTGCAGGAGCGCTCCCTTGAC
CTGCTGAAGTCCGGAACGTAGTCGGCACAGCCTGGTCGCCTTCCACCTCTGGGAGCTGGAGTCCACTGGGGTGGCCTG
ACTCCCCCAGTCCCCTTCCCGTGACCTGGTCAGGGTGAGCCCATGTGGAGTCAGCCTCGCAGGCCTCCCTGCCAGTAG
GGTCCGAGTGTGTTTCATCCTTCCCACTCTGTCGAGCCTGGGGGCTGGAGCGGAGACGGGAGGCCTGGCCTGTCTCGG
AACCTGTGAGCTGCACCAGGTAGAACGCCAGGGACCCCAGAATCATGTGCGTCAGTCCAAGGGGTCCCCTCCAGGAGT
AGTGAAGACTCCAGAAATGTCCCTTTCTTCTCCCCCATCCTACGAGTAATTGCATTTGCTTTTGTAATTCTTAATGAG
CAATATCTGCTAGAGAGTTTAGCTGTAACAGTTCTTTTTGATCATCTTTTTTTAATAATTAGAAACACCAAAAAAATC
CAGAAACTTGTTCTTCCAAAGCAGAGAGCATTATAATCACCAGGGCCAAAAGCTTCCCTCCCTGCTGTCATTGCTTCT
TCTGAGGCCTGAATCCAAAAGAAAAACAGCCATAGGCCCTTTCAGTGGCCGGGCTACCCGTGAGCCCTTCGGAGGACC
AGGGCTGGGGCAGCCTCTGGGCCCACATCCGGGGCCAGCTCCGGCGTGTGTTCAGTGTTAGCAGTGGGTCATGATGCT
CTTTCCCACCCAGCCTGGGATAGGGGCAGAGGAGGCGAGGAGGCCGTTGCCGCTGATGTTTGGCCGTGAACAGGTGGG
TGTCTGCGTGCGTCCACGTGCGTGTTTTCTGACTGACATGAAATCGACGCCCGAGTTAGCCTCACCCGGTGACCTCTA
GCCCTGCCCGGATGGAGCGGGGCCCACCCGGTTCAGTGTTTCTGGGGAGCTGGACAGTGGAGTGCAAAAGGCTTGCAG
AACTTGAAGCCTGCTCCTTCCCTTGCTACCACGGCCTCCTTTCCGTTTGATTTGTCACTGCTTCAATCAATAACAGCC
GCTCCAGAGTCAGTAGTCAATGAATATATGACCAAATATCACCAGGACTGTTACTCAATGTGTGCCGAGCCCTTGCCC
ATGCTGGGCTCCCGTGTATCTGGACACTGTAACGTGTGCTGTGTTTGCTCCCCTTCCCCTTCCTTCTTTGCCCTTTAC
TTGTCTTTCTGGGGTTTTTCTGTTTGGGTTTGGTTTGGTTTTTATTTCTCCTTTTGTGTTCCAAACATGAGGTTCTCT
CTACTGGTCCTCTTAACTGTGGTGTTGAGGCTTATATTTGTGTAATTTTTGGTGGGTGAAAGGAATTTTGCTAAGTAA
ATCTCTTCTGTGTTTGAACTGAAGTCTGTATTGTAACTATGTTTAAAGTAATTGTTCCAGAGACAAATATTTCTAGAC
ACTTTTTCTTTACAAACAAAAGCATTCGGAGGGAGGGGGATGGTGACTGAGATGAGAGGGGAGAGCTGAACAGATGAC
CCCTGCCCAGATCAGCCAGAAGCCACCCAAAGCAGTGGAGCCCAGGAGTCCCACTCCAAGCCAGCAAGCCGAATAGCT
GATGTGTTGCCACTTTCCAAGTCACTGCAAAACCAGGTTTTGTTCCGCCCAGTGGATTCTTGTTTTGCTTCCCCTCCC
CCCGAGATTATTACCACCATCCCGTGCTTTTAAGGAAAGGCAAGATTGATGTTTCCTTGAGGGGAGCCAGGAGGGGAT
GTGTGTGTGCAGAGCTGAAGAGCTGGGGAGAATGGGGCTGGGCCCACCCAAGCAGGAGGCTGGGACGCTCTGCTGTGG
GCACAGGTCAGGCTAATGTTGGCAGATGCAGCTCTTCCTGGACAGGCCAGGTGGTGGGCATTCTCTCTCCAAGGTGTG
CCCCGTGGGCATTACTGTTTAAGACACTTCCGTCACATCCCACCCCATCCTCCAGGGCTCAACACTGTGACATCTCTA
TTCCCCACCCTCCCCTTCCCAGGGCAATAAAATGACCATGGAGGGGGCTTGCACTCTCTTGGCTGTCACCCGATCGCC
AGCAAAACTTAGATGTGAGAAAACCCCTTCCCATTCCATGGCGAAAACATCTCCTTAGAAAAGCCATTACCCTCATTA
GGCATGGTTTTGGGCTCCCAAAACACCTGACAGCCCCTCCCTCCTCTGAGAGGCGGAGAGTGCTGACTGTAGTGACCA
TTGCATGCCGGGTGCAGCATCTGGAAGAGCTAGGCAGGGTGTCTGCCCCCTCCTGAGTTGAAGTCATGCTCCCCTGTG
CCAGCCCAGAGGCCGAGAGCTATGGACAGCATTGCCAGTAACACAGGCCACCCTGTGCAGAAGGGAGCTGGCTCCAGC
CTGGAAACCTGTCTGAGGTTGGGAGAGGTGCACTTGGGGCACAGGGAGAGGCCGGGACACACTTAGCTGGAGATGTCT
CTAAAAGCCCTGTATCGTATTCACCTTCAGTTTTTGTGTTTTGGGACAATTACTTTAGAAAATAAGTAGGTCGTTTTA
AAAACAAAAATTATTGATTGCTTTTTTGTAGTGTTCAGAAAAAAGGTTCTTTGTGTATAGCCAAATGACTGAAAGCAC
TGATATATTTAAAAACAAAAGGCAATTTATTAAGGAAATTTGTACCATTTCAGTAAACCTGTCTGAATGTACCTGTAT
ACGTTTCAAAAACACCCCCCCCCCACTGAATCCCTGTAACCTATTTATTATATAAAGAGTTTGCCTTATAAATTTACA
TAAAAATGTCCGTTTGTGTCTTTTGTTGTAAAAATCAAGTGATTTTTTCATAAGGTTCTTTTACTATTGGAAAAGATG
GGCAGCACGCAGTTTTATTTTATTTTTGTAAGTTTTTTAATACATGTGAAAGCAAAGAATACTCAGCATGCCTTTCTA
AGTGACGCGTTTGCACCTTTTGTTGGGAAGTACTGTATCCTGTGCTGTTAGCATTCTCGATAAATCTCTCTGTGAAAG
TGA
SEQ ID NO: 28 >NM_001386138.1 MECP2 [organism = Homo sapiens] [GeneID = 4204]
[transcript = 9]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGGGAAGAAAAGTCAGAAGACCAGGACCTCCAGGGCCTCAAGGACAAACCCCTCAAGTTTAAAAAGGTGAAGAAAG
ATAAGAAAGAAGAGAAAGAGGGCAAGCATGAGCCCGTGCAGCCATCAGCCCACCACTCTGCTGAGCCCGCAGAGGCAG
GCAAAGCAGAGACATCAGAAGGGTCAGGCTCCGCCCCGGCTGTGCCGGAAGCTTCTGCCTCCCCCAAACAGCGGCGCT
CCATCATCCGTGACCGGGGACCCATGTATGATGACCCCACCCTGCCTGAAGGCTGGACACGGAAGCTTAAGCAAAGGA
AATCTGGCCGCTCTGCTGGGAAGTATGATGTGTATTTGATCAATCCCCAGGGAAAAGCCTTTCGCTCTAAAGTGGAGT
TGATTGCGTACTTCGAAAAGCTCCAGGAACTGGCAGAGGCCGGGGACGCCCCAAAGGGAGCGGCACCACGAGACCCAA
GGCGGCCACGTCAGAGGGTGTGCAGGTGAAAAGGGTCCTGGAGAAAAGTCCTGGGAAGCTCCTTGTCAAGATGCCTTT
TCAAACTTCGCCAGGGGGCAAGGCTGAGGGGGGTGGGGCCACCACATCCACCCAGGTCATGGTGATCAAACGCCCCGG
CAGGAAGCGAAAAGCTGAGGCCGACCCTCAGGCCATTCCCAAGAAACGGGGCCGAAAGCCGGGGAGTGTGGTGGCAGC
CGCTGCCGCCGAGGCCAAAAAGAAAGCCGTGAAGGAGTCTTCTATCCGATCTGTGCAGGAGACCGTACTCCCCATCAA
GAAGCGCAAGACCCGGGAGACGGTCAGCATCGAGGTCAAGGAAGTGGTGAAGCCCCTGCTGGTGTCCACCCTCGGTGA
GAAGAGCGGGAAAGGACTGAAGACCTGTAAGAGCCCTGGGCGGAAAAGCAAGGAGAGCAGCCCCAAGGGGCGCAGCAG
CAGCGCCTCCTCACCCCCCAAGAAGGAGCACCACCACCATCACCACCACTCAGAGTCCCCAAAGGCCCCCGTGCCACT
GCTCCCACCCCTGCCCCCACCTCCACCTGAGCCCGAGAGCTCCGAGGACCCCACCAGCCCCCCTGAGCCCCAGGACTT
GAGCAGCAGCGTCTGCAAAGAGGAGAAGATGCCCAGAGGAGGCTCACTGGAGAGCGACGGCTGCCCCAAGGAGCCAGC
TAAGACTCAGCCCGCGGTTGCCACCGCCGCCACGGCCGCAGAAAAGTACAAACACCGAGGGGAGGGAGAGCGCAAAGA
CATTGTTTCATCCTCCATGCCAAGGCCAAACAGAGAGGAGCCTGTGGACAGCCGGACGCCCGTGACCGAGAGAGTTAG
CTGACTTTACACGGAGCGGATTGCAAAGCAAACCAACAAGAATAAAGGCAGCTGTTGTCTCTTCTCCTTATGGGTAGG
GCTCTGACAAAGCTTCCCGATTAACTGAAATAAAAAATATTTTTTTTTCTTTCAGTAAACTTAGAGTTTCGTGGCTTC
AGGGTGGGAGTAGTTGGAGCATTGGGGATGTTTTTCTTACCGACAAGCACAGTCAGGTTGAAGACCTAACCAGGGCCA
GAAGTAGCTTTGCACTTTTCTAAACTAGGCTCCTTCAACAAGGCTTGCTGCAGATACTACTGACCAGACAAGCTGTTG
ACCAGGCACCTCCCCTCCCGCCCAAACCTTTCCCCCATGTGGTCGTTAGAGACAGAGCGACAGAGCAGTTGAGAGGAC
ACTCCCGTTTTCGGTGCCATCAGTGCCCCGTCTACAGCTCCCCCAGCTCCCCCCACCTCCCCCACTCCCAACCACGTT
GGGACAGGGAGGTGTGAGGCAGGAGAGACAGTTGGATTCTTTAGAGAAGATGGATATGACCAGTGGCTATGGCCTGTG
CGATCCCACCCGTGGTGGCTCAAGTCTGGCCCCACACCAGCCCCAATCCAAAACTGGCAAGGACGCTTCACAGGACAG
GAAAGTGGCACCTGTCTGCTCCAGCTCTGGCATGGCTAGGAGGGGGGAGTCCCTTGAACTACTGGGTGTAGACTGGCC
TGAACCACAGGAGAGGATGGCCCAGGGTGAGGTGGCATGGTCCATTCTCAAGGGACGTCCTCCAACGGGTGGCGCTAG
AGGCCATGGAGGCAGTAGGACAAGGTGCAGGCAGGCTGGCCTGGGGTCAGGCCGGGCAGAGCACAGCGGGGTGAGAGG
GATTCCTAATCACTCAGAGCAGTCTGTGACTTAGTGGACAGGGGAGGGGGCAAAGGGGGAGGAGAAGAAAATGTTCTT
CCAGTTACTTTCCAATTCTCCTTTAGGGACAGCTTAGAATTATTTGCACTATTGAGTCTTCATGTTCCCACTTCAAAA
CAAACAGATGCTCTGAGAGCAAACTGGCTTGAATTGGTGACATTTAGTCCCTCAAGCCACCAGATGTGACAGTGTTGA
GAACTACCTGGATTTGTATATATACCTGCGCTTGTTTTAAAGTGGGCTCAGCACATAGGGTTCCCACGAAGCTCCGAA
ACTCTAAGTGTTTGCTGCAATTTTATAAGGACTTCCTGATTGGTTTCTCTTCTCCCCTTCCATTTCTGCCTTTTGTTC
ATTTCATCCTTTCACTTCTTTCCCTTCCTCCATCCTCCTCCTTCCTAGTTCATCCCTTCTCTTCCAGGCAGCCGCGGT
GCCCAACCACACTTGTCGGCTCCAGTCCCCAGAACTCTGCCTGCCCTTTGTCCTCCTGCTGCCAGTACCAGCCCCACC
CTGTTTTGAGCCCTGAGGAGGCCTTGGGCTCTGCTGAGTCCGACCTGGCCTGTCTGTGAAGAGCAAGAGAGCAGCAAG
GTCTTGCTCTCCTAGGTAGCCCCCTCTTCCCTGGTAAGAAAAAGCAAAAGGCATTTCCCACCCTGAACAACGAGCCTT
TTCACCCTTCTACTCTAGAGAAGTGGACTGGAGGAGCTGGGCCCGATTTGGTAGTTGAGGAAAGCACAGAGGCCTCCT
GTGGCCTGCCAGTCATCGAGTGGCCCAACAGGGGCTCCATGCCAGCCGACCTTGACCTCACTCAGAAGTCCAGAGTCT
AGCGTAGTGCAGCAGGGCAGTAGCGGTACCAATGCAGAACTCCCAAGACCCGAGCTGGGACCAGTACCTGGGTCCCCA
GCCCTTCCTCTGCTCCCCCTTTTCCCTCGGAGTTCTTCTTGAATGGCAATGTTTTGCTTTTGCTCGATGCAGACAGGG
GGCCAGAACACCACACATTTCACTGTCTGTCTGGTCCATAGCTGTGGTGTAGGGGCTTAGAGGCATGGGCTTGCTGTG
GGTTTTTAATTGATCAGTTTTCATGTGGGATCCCATCTTTTTAACCTCTGTTCAGGAAGTCCTTATCTAGCTGCATAT
CTTCATCATATTGGTATATCCTTTTCTGTGTTTACAGAGATGTCTCTTATATCTAAATCTGTCCAACTGAGAAGTACC
TTATCAAAGTAGCAAATGAGACAGCAGTCTTATGCTTCCAGAAACACCCACAGGCATGTCCCATGTGAGCTGCTGCCA
TGAACTGTCAAGTGTGTGTTGTCTTGTGTATTTCAGTTATTGTCCCTGGCTTCCTTACTATGGTGTAATCATGAAGGA
GTGAAACATCATAGAAACTGTCTAGCACTTCCTTGCCAGTCTTTAGTGATCAGGAACCATAGTTGACAGTTCCAATCA
GTAGCTTAAGAAAAAACCGTGTTTGTCTCTTCTGGAATGGTTAGAAGTGAGGGAGTTTGCCCCGTTCTGTTTGTAGAG
TCTCATAGTTGGACTTTCTAGCATATATGTGTCCATTTCCTTATGCTGTAAAAGCAAGTCCTGCAACCAAACTCCCAT
CAGCCCAATCCCTGATCCCTGATCCCTTCCACCTGCTCTGCTGATGACCCCCCCAGCTTCACTTCTGACTCTTCCCCA
GGAAGGGAAGGGGGGTCAGAAGAGAGGGTGAGTCCTCCAGAACTCTTCCTCCAAGGACAGAAGGCTCCTGCCCCCATA
GTGGCCTCGAACTCCTGGCACTACCAAAGGACACTTATCCACGAGAGCGCAGCATCCGACCAGGTTGTCACTGAGAAG
ATGTTTATTTTGGTCAGTTGGGTTTTTATGTATTATACTTAGTCAAATGTAATGTGGCTTCTGGAATCATTGTCCAGA
GCTGCTTCCCCGTCACCTGGGCGTCATCTGGTCCTGGTAAGAGGAGTGCGTGGCCCACCAGGCCCCCCTGTCACCCAT
GACAGTTCATTCAGGGCCGATGGGGCAGTCGTGGTTGGGAACACAGCATTTCAAGCGTCACTTTATTTCATTCGGGCC
CCACCTGCAGCTCCCTCAAAGAGGCAGTTGCCCAGCCTCTTTCCCTTCCAGTTTATTCCAGAGCTGCCAGTGGGGCCT
GAGGCTCCTTAGGGTTTTCTCTCTATTTCCCCCTTTCTTCCTCATTCCCTCGTCTTTCCCAAAGGCATCACGAGTCAG
TCGCCTTTCAGCAGGCAGCCTTGGCGGTTTATCGCCCTGGCAGGCAGGGGCCCTGCAGCTCTCATGCTGCCCCTGCCT
TGGGGTCAGGTTGACAGGAGGTTGGAGGGAAAGCCTTAAGCTGCAGGATTCTCACCAGCTGTGTCCGGCCCAGTTTTG
GGGTGTGACCTCAATTTCAATTTTGTCTGTACTTGAACATTATGAAGATGGGGGCCTCTTTCAGTGAATTTGTGAACA
GCAGAATTGACCGACAGCTTTCCAGTACCCATGGGGCTAGGTCATTAAGGCCACATCCACAGTCTCCCCCACCCTTGT
TCCAGTTGTTAGTTACTACCTCCTCTCCTGACAATACTGTATGTCGTCGAGCTCCCCCCAGGTCTACCCCTCCCGGCC
CTGCCTGCTGGTGGGCTTGTCATAGCCAGTGGGATTGCCGGTCTTGACAGCTCAGTGAGCTGGAGATACTTGGTCACA
GCCAGGCGCTAGCACAGCTCCCTTCTGTTGATGCTGTATTCCCATATCAAAAGACACAGGGGACACCCAGAAACGCCA
CATCCCCCAATCCATCAGTGCCAAACTAGCCAACGGCCCCAGCTTCTCAGCTCGCTGGATGGCGGAAGCTGCTACTCG
TGAGCGCCAGTGCGGGTGCAGACAATCTTCTGTTGGGTGGCATCATTCCAGGCCCGAAGCATGAACAGTGCACCTGGG
ACAGGGAGCAGCCCCAAATTGTCACCTGCTTCTCTGCCCAGCTTTTCATTGCTGTGACAGTGATGGCGAAAGAGGGTA
ATAACCAGACACAAACTGCCAAGTTGGGTGGAGAAAGGAGTTTCTTTAGCTGACAGAATCTCTGAATTTTAAATCACT
TAGTAAGCGGCTCAAGCCCAGGAGGGAGCAGAGGGATACGAGCGGAGTCCCCTGCGCGGGACCATCTGGAATTGGTTT
AGCCCAAGTGGAGCCTGACAGCCAGAACTCTGTGTCCCCCGTCTAACCACAGCTCCTTTTCCAGAGCATTCCAGTCAG
GCTCTCTGGGCTGACTGGGCCAGGGGAGGTTACAGGTACCAGTTCTTTAAGAAGATCTTTGGGCATATACATTTTTAG
CCTGTGTCATTGCCCCAAATGGATTCCTGTTTCAAGTTCACACCTGCAGATTCTAGGACCTGTGTCCTAGACTTCAGG
GAGTCAGCTGTTTCTAGAGTTCCTACCATGGAGTGGGTCTGGAGGACCTGCCCGGTGGGGGGGCAGAGCCCTGCTCCC
TCCGGGTCTTCCTACTCTTCTCTCTGCTCTGACGGGATTTGTTGATTCTCTCCATTTTGGTGTCTTTCTCTTTTAGAT
ATTGTATCAATCTTTAGAAAAGGCATAGTCTACTTGTTATAAATCGTTAGGATACTGCCTCCCCCAGGGTCTAAAATT
ACATATTAGAGGGGAAAAGCTGAACACTGAAGTCAGTTCTCAACAATTTAGAAGGAAAACCTAGAAAACATTTGGCAG
AAAATTACATTTCGATGTTTTTGAATGAATACGAGCAAGCTTTTACAACAGTGCTGATCTAAAAATACTTAGCACTTG
GCCTGAGATGCCTGGTGAGCATTACAGGCAAGGGGAATCTGGAGGTAGCCGACCTGAGGACATGGCTTCTGAACCTGT
CTTTTGGGAGTGGTATGGAAGGTGGAGCGTTCACCAGTGACCTGGAAGGCCCAGCACCACCCTCCTTCCCACTCTTCT
CATCTTGACAGAGCCTGCCCCAGCGCTGACGTGTCAGGAAAACACCCAGGGAACTAGGAAGGCACTTCTGCCTGAGGG
GCAGCCTGCCTTGCCCACTCCTGCTCTGCTCGCCTCGGATCAGCTGAGCCTTCTGAGCTGGCCTCTCACTGCCTCCCC
AAGGCCCCCTGCCTGCCCTGTCAGGAGGCAGAAGGAAGCAGGTGTGAGGGCAGTGCAAGGAGGGAGCACAACCCCCAG
CTCCCGCTCCGGGCTCCGACTTGTGCACAGGCAGAGCCCAGACCCTGGAGGAAATCCTACCTTTGAATTCAAGAACAT
TTGGGGAATTTGGAAATCTCTTTGCCCCCAAACCCCCATTCTGTCCTACCTTTAATCAGGTCCTGCTCAGCAGTGAGA
GCAGATGAGGTGAAAAGGCCAAGAGGTTTGGCTCCTGCCCACTGATAGCCCCTCTCCCCGCAGTGTTTGTGTGTCAAG
TGGCAAAGCTGTTCTTCCTGGTGACCCTGATTATATCCAGTAACACATAGACTGTGCGCATAGGCCTGCTTTGTCTCC
TCTATCCTGGGCTTTTGTTTTGCTTTTTAGTTTTGCTTTTAGTTTTTCTGTCCCTTTTATTTAACGCACCGACTAGAC
ACACAAAGCAGTTGAATTTTTATATATATATCTGTATATTGCACAATTATAAACTCATTTTGCTTGTGGCTCCACACA
CACAAAAAAAGACCTGTTAAAATTATACCTGTTGCTTAATTACAATATTTCTGATAACCATAGCATAGGACAAGGGAA
AATAAAAAAAGAAAAAAAAGAAAAAAAAACGACAAATCTGTCTGCTGGTCACTTCTTCTGTCCAAGCAGATTCGTGGT
CTTTTCCTCGCTTCTTTCAAGGGCTTTCCTGTGCCAGGTGAAGGAGGCTCCAGGCAGCACCCAGGTTTTGCACTCTTG
TTTCTCCCGTGCTTGTGAAAGAGGTCCCAAGGTTCTGGGTGCAGGAGCGCTCCCTTGACCTGCTGAAGTCCGGAACGT
AGTCGGCACAGCCTGGTCGCCTTCCACCTCTGGGAGCTGGAGTCCACTGGGGTGGCCTGACTCCCCCAGTCCCCTTCC
CGTGACCTGGTCAGGGTGAGCCCATGTGGAGTCAGCCTCGCAGGCCTCCCTGCCAGTAGGGTCCGAGTGTGTTTCATC
CTTCCCACTCTGTCGAGCCTGGGGGCTGGAGCGGAGACGGGAGGCCTGGCCTGTCTCGGAACCTGTGAGCTGCACCAG
GTAGAACGCCAGGGACCCCAGAATCATGTGCGTCAGTCCAAGGGGTCCCCTCCAGGAGTAGTGAAGACTCCAGAAATG
TCCCTTTCTTCTCCCCCATCCTACGAGTAATTGCATTTGCTTTTGTAATTCTTAATGAGCAATATCTGCTAGAGAGTT
TAGCTGTAACAGTTCTTTTTGATCATCTTTTTTTAATAATTAGAAACACCAAAAAAATCCAGAAACTTGTTCTTCCAA
AGCAGAGAGCATTATAATCACCAGGGCCAAAAGCTTCCCTCCCTGCTGTCATTGCTTCTTCTGAGGCCTGAATCCAAA
AGAAAAACAGCCATAGGCCCTTTCAGTGGCCGGGCTACCCGTGAGCCCTTCGGAGGACCAGGGCTGGGGCAGCCTCTG
GGCCCACATCCGGGGCCAGCTCCGGCGTGTGTTCAGTGTTAGCAGTGGGTCATGATGCTCTTTCCCACCCAGCCTGGG
ATAGGGGCAGAGGAGGCGAGGAGGCCGTTGCCGCTGATGTTTGGCCGTGAACAGGTGGGTGTCTGCGTGCGTCCACGT
GCGTGTTTTCTGACTGACATGAAATCGACGCCCGAGTTAGCCTCACCCGGTGACCTCTAGCCCTGCCCGGATGGAGCG
GGGCCCACCCGGTTCAGTGTTTCTGGGGAGCTGGACAGTGGAGTGCAAAAGGCTTGCAGAACTTGAAGCCTGCTCCTT
CCCTTGCTACCACGGCCTCCTTTCCGTTTGATTTGTCACTGCTTCAATCAATAACAGCCGCTCCAGAGTCAGTAGTCA
ATGAATATATGACCAAATATCACCAGGACTGTTACTCAATGTGTGCCGAGCCCTTGCCCATGCTGGGCTCCCGTGTAT
CTGGACACTGTAACGTGTGCTGTGTTTGCTCCCCTTCCCCTTCCTTCTTTGCCCTTTACTTGTCTTTCTGGGGTTTTT
CTGTTTGGGTTTGGTTTGGTTTTTATTTCTCCTTTTGTGTTCCAAACATGAGGTTCTCTCTACTGGTCCTCTTAACTG
TGGTGTTGAGGCTTATATTTGTGTAATTTTTGGTGGGTGAAAGGAATTTTGCTAAGTAAATCTCTTCTGTGTTTGAAC
TGAAGTCTGTATTGTAACTATGTTTAAAGTAATTGTTCCAGAGACAAATATTTCTAGACACTTTTTCTTTACAAACAA
AAGCATTCGGAGGGAGGGGGATGGTGACTGAGATGAGAGGGGAGAGCTGAACAGATGACCCCTGCCCAGATCAGCCAG
AAGCCACCCAAAGCAGTGGAGCCCAGGAGTCCCACTCCAAGCCAGCAAGCCGAATAGCTGATGTGTTGCCACTTTCCA
AGTCACTGCAAAACCAGGTTTTGTTCCGCCCAGTGGATTCTTGTTTTGCTTCCCCTCCCCCCGAGATTATTACCACCA
TCCCGTGCTTTTAAGGAAAGGCAAGATTGATGTTTCCTTGAGGGGAGCCAGGAGGGGATGTGTGTGTGCAGAGCTGAA
GAGCTGGGGAGAATGGGGCTGGGCCCACCCAAGCAGGAGGCTGGGACGCTCTGCTGTGGGCACAGGTCAGGCTAATGT
TGGCAGATGCAGCTCTTCCTGGACAGGCCAGGTGGTGGGCATTCTCTCTCCAAGGTGTGCCCCGTGGGCATTACTGTT
TAAGACACTTCCGTCACATCCCACCCCATCCTCCAGGGCTCAACACTGTGACATCTCTATTCCCCACCCTCCCCTTCC
CAGGGCAATAAAATGACCATGGAGGGGGCTTGCACTCTCTTGGCTGTCACCCGATCGCCAGCAAAACTTAGATGTGAG
AAAACCCCTTCCCATTCCATGGCGAAAACATCTCCTTAGAAAAGCCATTACCCTCATTAGGCATGGTTTTGGGCTCCC
AAAACACCTGACAGCCCCTCCCTCCTCTGAGAGGCGGAGAGTGCTGACTGTAGTGACCATTGCATGCCGGGTGCAGCA
TCTGGAAGAGCTAGGCAGGGTGTCTGCCCCCTCCTGAGTTGAAGTCATGCTCCCCTGTGCCAGCCCAGAGGCCGAGAG
CTATGGACAGCATTGCCAGTAACACAGGCCACCCTGTGCAGAAGGGAGCTGGCTCCAGCCTGGAAACCTGTCTGAGGT
TGGGAGAGGTGCACTTGGGGCACAGGGAGAGGCCGGGACACACTTAGCTGGAGATGTCTCTAAAAGCCCTGTATCGTA
TTCACCTTCAGTTTTTGTGTTTTGGGACAATTACTTTAGAAAATAAGTAGGTCGTTTTAAAAACAAAAATTATTGATT
GCTTTTTTGTAGTGTTCAGAAAAAAGGTTCTTTGTGTATAGCCAAATGACTGAAAGCACTGATATATTTAAAAACAAA
AGGCAATTTATTAAGGAAATTTGTACCATTTCAGTAAACCTGTCTGAATGTACCTGTATACGTTTCAAAAACACCCCC
CCCCCACTGAATCCCTGTAACCTATTTATTATATAAAGAGTTTGCCTTATAAATTTACATAAAAATGTCCGTTTGTGT
CTTTTGTTGTAAAAATCAAGTGATTTTTTCATAAGGTTCTTTTACTATTGGAAAAGATGGGCAGCACGCAGTTTTATT
TTATTTTTGTAAGTTTTTTAATACATGTGAAAGCAAAGAATACTCAGCATGCCTTTCTAAGTGACGCGTTTGCACCTT
TTGTTGGGAAGTACTGTATCCTGTGCTGTTAGCATTCTCGATAAATCTCTCTGTGAAAGTGA
SEQ ID NO: 29 >NM_004992.4 MECP2 [organism = Homo sapiens] [GeneID = 4204]
[transcript = 1]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGGGAAGAAAAGTCAGAAGACCAGGACCTCCAGGGCCTCAAGGACAAACCCCTCAAGTTTAAAAAGGTGAAGAAAG
ATAAGAAAGAAGAGAAAGAGGGCAAGCATGAGCCCGTGCAGCCATCAGCCCACCACTCTGCTGAGCCCGCAGAGGCAG
GCAAAGCAGAGACATCAGAAGGGTCAGGCTCCGCCCCGGCTGTGCCGGAAGCTTCTGCCTCCCCCAAACAGCGGCGCT
CCATCATCCGTGACCGGGGACCCATGTATGATGACCCCACCCTGCCTGAAGGCTGGACACGGAAGCTTAAGCAAAGGA
AATCTGGCCGCTCTGCTGGGAAGTATGATGTGTATTTGATCAATCCCCAGGGAAAAGCCTTTCGCTCTAAAGTGGAGT
TGATTGCGTACTTCGAAAAGGTAGGCGACACATCCCTGGACCCTAATGATTTTGACTTCACGGTAACTGGGAGAGGGA
GCCCCTCCCGGCGAGAGCAGAAACCACCTAAGAAGCCCAAATCTCCCAAAGCTCCAGGAACTGGCAGAGGCCGGGGAC
GCCCCAAAGGGAGCGGCACCACGAGACCCAAGGCGGCCACGTCAGAGGGTGTGCAGGTGAAAAGGGTCCTGGAGAAAA
GTCCTGGGAAGCTCCTTGTCAAGATGCCTTTTCAAACTTCGCCAGGGGGCAAGGCTGAGGGGGGTGGGGCCACCACAT
CCACCCAGGTCATGGTGATCAAACGCCCCGGCAGGAAGCGAAAAGCTGAGGCCGACCCTCAGGCCATTCCCAAGAAAC
GGGGCCGAAAGCCGGGGAGTGTGGTGGCAGCCGCTGCCGCCGAGGCCAAAAAGAAAGCCGTGAAGGAGTCTTCTATCC
GATCTGTGCAGGAGACCGTACTCCCCATCAAGAAGCGCAAGACCCGGGAGACGGTCAGCATCGAGGTCAAGGAAGTGG
TGAAGCCCCTGCTGGTGTCCACCCTCGGTGAGAAGAGCGGGAAAGGACTGAAGACCTGTAAGAGCCCTGGGCGGAAAA
GCAAGGAGAGCAGCCCCAAGGGGCGCAGCAGCAGCGCCTCCTCACCCCCCAAGAAGGAGCACCACCACCATCACCACC
ACTCAGAGTCCCCAAAGGCCCCCGTGCCACTGCTCCCACCCCTGCCCCCACCTCCACCTGAGCCCGAGAGCTCCGAGG
ACCCCACCAGCCCCCCTGAGCCCCAGGACTTGAGCAGCAGCGTCTGCAAAGAGGAGAAGATGCCCAGAGGAGGCTCAC
TGGAGAGCGACGGCTGCCCCAAGGAGCCAGCTAAGACTCAGCCCGCGGTTGCCACCGCCGCCACGGCCGCAGAAAAGT
ACAAACACCGAGGGGAGGGAGAGCGCAAAGACATTGTTTCATCCTCCATGCCAAGGCCAAACAGAGAGGAGCCTGTGG
ACAGCCGGACGCCCGTGACCGAGAGAGTTAGCTGACTTTACACGGAGCGGATTGCAAAGCAAACCAACAAGAATAAAG
GCAGCTGTTGTCTCTTCTCCTTATGGGTAGGGCTCTGACAAAGCTTCCCGATTAACTGAAATAAAAAATATTTTTTTT
TCTTTCAGTAAACTTAGAGTTTCGTGGCTTCAGGGTGGGAGTAGTTGGAGCATTGGGGATGTTTTTCTTACCGACAAG
CACAGTCAGGTTGAAGACCTAACCAGGGCCAGAAGTAGCTTTGCACTTTTCTAAACTAGGCTCCTTCAACAAGGCTTG
CTGCAGATACTACTGACCAGACAAGCTGTTGACCAGGCACCTCCCCTCCCGCCCAAACCTTTCCCCCATGTGGTCGTT
AGAGACAGAGCGACAGAGCAGTTGAGAGGACACTCCCGTTTTCGGTGCCATCAGTGCCCCGTCTACAGCTCCCCCAGC
TCCCCCCACCTCCCCCACTCCCAACCACGTTGGGACAGGGAGGTGTGAGGCAGGAGAGACAGTTGGATTCTTTAGAGA
AGATGGATATGACCAGTGGCTATGGCCTGTGCGATCCCACCCGTGGTGGCTCAAGTCTGGCCCCACACCAGCCCCAAT
CCAAAACTGGCAAGGACGCTTCACAGGACAGGAAAGTGGCACCTGTCTGCTCCAGCTCTGGCATGGCTAGGAGGGGGG
AGTCCCTTGAACTACTGGGTGTAGACTGGCCTGAACCACAGGAGAGGATGGCCCAGGGTGAGGTGGCATGGTCCATTC
TCAAGGGACGTCCTCCAACGGGTGGCGCTAGAGGCCATGGAGGCAGTAGGACAAGGTGCAGGCAGGCTGGCCTGGGGT
CAGGCCGGGCAGAGCACAGCGGGGTGAGAGGGATTCCTAATCACTCAGAGCAGTCTGTGACTTAGTGGACAGGGGAGG
GGGCAAAGGGGGAGGAGAAGAAAATGTTCTTCCAGTTACTTTCCAATTCTCCTTTAGGGACAGCTTAGAATTATTTGC
ACTATTGAGTCTTCATGTTCCCACTTCAAAACAAACAGATGCTCTGAGAGCAAACTGGCTTGAATTGGTGACATTTAG
TCCCTCAAGCCACCAGATGTGACAGTGTTGAGAACTACCTGGATTTGTATATATACCTGCGCTTGTTTTAAAGTGGGC
TCAGCACATAGGGTTCCCACGAAGCTCCGAAACTCTAAGTGTTTGCTGCAATTTTATAAGGACTTCCTGATTGGTTTC
TCTTCTCCCCTTCCATTTCTGCCTTTTGTTCATTTCATCCTTTCACTTCTTTCCCTTCCTCCATCCTCCTCCTTCCTA
GTTCATCCCTTCTCTTCCAGGCAGCCGCGGTGCCCAACCACACTTGTCGGCTCCAGTCCCCAGAACTCTGCCTGCCCT
TTGTCCTCCTGCTGCCAGTACCAGCCCCACCCTGTTTTGAGCCCTGAGGAGGCCTTGGGCTCTGCTGAGTCCGACCTG
GCCTGTCTGTGAAGAGCAAGAGAGCAGCAAGGTCTTGCTCTCCTAGGTAGCCCCCTCTTCCCTGGTAAGAAAAAGCAA
AAGGCATTTCCCACCCTGAACAACGAGCCTTTTCACCCTTCTACTCTAGAGAAGTGGACTGGAGGAGCTGGGCCCGAT
TTGGTAGTTGAGGAAAGCACAGAGGCCTCCTGTGGCCTGCCAGTCATCGAGTGGCCCAACAGGGGCTCCATGCCAGCC
GACCTTGACCTCACTCAGAAGTCCAGAGTCTAGCGTAGTGCAGCAGGGCAGTAGCGGTACCAATGCAGAACTCCCAAG
ACCCGAGCTGGGACCAGTACCTGGGTCCCCAGCCCTTCCTCTGCTCCCCCTTTTCCCTCGGAGTTCTTCTTGAATGGC
AATGTTTTGCTTTTGCTCGATGCAGACAGGGGGCCAGAACACCACACATTTCACTGTCTGTCTGGTCCATAGCTGTGG
TGTAGGGGCTTAGAGGCATGGGCTTGCTGTGGGTTTTTAATTGATCAGTTTTCATGTGGGATCCCATCTTTTTAACCT
CTGTTCAGGAAGTCCTTATCTAGCTGCATATCTTCATCATATTGGTATATCCTTTTCTGTGTTTACAGAGATGTCTCT
TATATCTAAATCTGTCCAACTGAGAAGTACCTTATCAAAGTAGCAAATGAGACAGCAGTCTTATGCTTCCAGAAACAC
CCACAGGCATGTCCCATGTGAGCTGCTGCCATGAACTGTCAAGTGTGTGTTGTCTTGTGTATTTCAGTTATTGTCCCT
GGCTTCCTTACTATGGTGTAATCATGAAGGAGTGAAACATCATAGAAACTGTCTAGCACTTCCTTGCCAGTCTTTAGT
GATCAGGAACCATAGTTGACAGTTCCAATCAGTAGCTTAAGAAAAAACCGTGTTTGTCTCTTCTGGAATGGTTAGAAG
TGAGGGAGTTTGCCCCGTTCTGTTTGTAGAGTCTCATAGTTGGACTTTCTAGCATATATGTGTCCATTTCCTTATGCT
GTAAAAGCAAGTCCTGCAACCAAACTCCCATCAGCCCAATCCCTGATCCCTGATCCCTTCCACCTGCTCTGCTGATGA
CCCCCCCAGCTTCACTTCTGACTCTTCCCCAGGAAGGGAAGGGGGGTCAGAAGAGAGGGTGAGTCCTCCAGAACTCTT
CCTCCAAGGACAGAAGGCTCCTGCCCCCATAGTGGCCTCGAACTCCTGGCACTACCAAAGGACACTTATCCACGAGAG
CGCAGCATCCGACCAGGTTGTCACTGAGAAGATGTTTATTTTGGTCAGTTGGGTTTTTATGTATTATACTTAGTCAAA
TGTAATGTGGCTTCTGGAATCATTGTCCAGAGCTGCTTCCCCGTCACCTGGGCGTCATCTGGTCCTGGTAAGAGGAGT
GCGTGGCCCACCAGGCCCCCCTGTCACCCATGACAGTTCATTCAGGGCCGATGGGGCAGTCGTGGTTGGGAACACAGC
ATTTCAAGCGTCACTTTATTTCATTCGGGCCCCACCTGCAGCTCCCTCAAAGAGGCAGTTGCCCAGCCTCTTTCCCTT
CCAGTTTATTCCAGAGCTGCCAGTGGGGCCTGAGGCTCCTTAGGGTTTTCTCTCTATTTCCCCCTTTCTTCCTCATTC
CCTCGTCTTTCCCAAAGGCATCACGAGTCAGTCGCCTTTCAGCAGGCAGCCTTGGCGGTTTATCGCCCTGGCAGGCAG
GGGCCCTGCAGCTCTCATGCTGCCCCTGCCTTGGGGTCAGGTTGACAGGAGGTTGGAGGGAAAGCCTTAAGCTGCAGG
ATTCTCACCAGCTGTGTCCGGCCCAGTTTTGGGGTGTGACCTCAATTTCAATTTTGTCTGTACTTGAACATTATGAAG
ATGGGGGCCTCTTTCAGTGAATTTGTGAACAGCAGAATTGACCGACAGCTTTCCAGTACCCATGGGGCTAGGTCATTA
AGGCCACATCCACAGTCTCCCCCACCCTTGTTCCAGTTGTTAGTTACTACCTCCTCTCCTGACAATACTGTATGTCGT
CGAGCTCCCCCCAGGTCTACCCCTCCCGGCCCTGCCTGCTGGTGGGCTTGTCATAGCCAGTGGGATTGCCGGTCTTGA
CAGCTCAGTGAGCTGGAGATACTTGGTCACAGCCAGGCGCTAGCACAGCTCCCTTCTGTTGATGCTGTATTCCCATAT
CAAAAGACACAGGGGACACCCAGAAACGCCACATCCCCCAATCCATCAGTGCCAAACTAGCCAACGGCCCCAGCTTCT
CAGCTCGCTGGATGGCGGAAGCTGCTACTCGTGAGCGCCAGTGCGGGTGCAGACAATCTTCTGTTGGGTGGCATCATT
CCAGGCCCGAAGCATGAACAGTGCACCTGGGACAGGGAGCAGCCCCAAATTGTCACCTGCTTCTCTGCCCAGCTTTTC
ATTGCTGTGACAGTGATGGCGAAAGAGGGTAATAACCAGACACAAACTGCCAAGTTGGGTGGAGAAAGGAGTTTCTTT
AGCTGACAGAATCTCTGAATTTTAAATCACTTAGTAAGCGGCTCAAGCCCAGGAGGGAGCAGAGGGATACGAGCGGAG
TCCCCTGCGCGGGACCATCTGGAATTGGTTTAGCCCAAGTGGAGCCTGACAGCCAGAACTCTGTGTCCCCCGTCTAAC
CACAGCTCCTTTTCCAGAGCATTCCAGTCAGGCTCTCTGGGCTGACTGGGCCAGGGGAGGTTACAGGTACCAGTTCTT
TAAGAAGATCTTTGGGCATATACATTTTTAGCCTGTGTCATTGCCCCAAATGGATTCCTGTTTCAAGTTCACACCTGC
AGATTCTAGGACCTGTGTCCTAGACTTCAGGGAGTCAGCTGTTTCTAGAGTTCCTACCATGGAGTGGGTCTGGAGGAC
CTGCCCGGTGGGGGGGCAGAGCCCTGCTCCCTCCGGGTCTTCCTACTCTTCTCTCTGCTCTGACGGGATTTGTTGATT
CTCTCCATTTTGGTGTCTTTCTCTTTTAGATATTGTATCAATCTTTAGAAAAGGCATAGTCTACTTGTTATAAATCGT
TAGGATACTGCCTCCCCCAGGGTCTAAAATTACATATTAGAGGGGAAAAGCTGAACACTGAAGTCAGTTCTCAACAAT
TTAGAAGGAAAACCTAGAAAACATTTGGCAGAAAATTACATTTCGATGTTTTTGAATGAATACGAGCAAGCTTTTACA
ACAGTGCTGATCTAAAAATACTTAGCACTTGGCCTGAGATGCCTGGTGAGCATTACAGGCAAGGGGAATCTGGAGGTA
GCCGACCTGAGGACATGGCTTCTGAACCTGTCTTTTGGGAGTGGTATGGAAGGTGGAGCGTTCACCAGTGACCTGGAA
GGCCCAGCACCACCCTCCTTCCCACTCTTCTCATCTTGACAGAGCCTGCCCCAGCGCTGACGTGTCAGGAAAACACCC
AGGGAACTAGGAAGGCACTTCTGCCTGAGGGGCAGCCTGCCTTGCCCACTCCTGCTCTGCTCGCCTCGGATCAGCTGA
GCCTTCTGAGCTGGCCTCTCACTGCCTCCCCAAGGCCCCCTGCCTGCCCTGTCAGGAGGCAGAAGGAAGCAGGTGTGA
GGGCAGTGCAAGGAGGGAGCACAACCCCCAGCTCCCGCTCCGGGCTCCGACTTGTGCACAGGCAGAGCCCAGACCCTG
GAGGAAATCCTACCTTTGAATTCAAGAACATTTGGGGAATTTGGAAATCTCTTTGCCCCCAAACCCCCATTCTGTCCT
ACCTTTAATCAGGTCCTGCTCAGCAGTGAGAGCAGATGAGGTGAAAAGGCCAAGAGGTTTGGCTCCTGCCCACTGATA
GCCCCTCTCCCCGCAGTGTTTGTGTGTCAAGTGGCAAAGCTGTTCTTCCTGGTGACCCTGATTATATCCAGTAACACA
TAGACTGTGCGCATAGGCCTGCTTTGTCTCCTCTATCCTGGGCTTTTGTTTTGCTTTTTAGTTTTGCTTTTAGTTTTT
CTGTCCCTTTTATTTAACGCACCGACTAGACACACAAAGCAGTTGAATTTTTATATATATATCTGTATATTGCACAAT
TATAAACTCATTTTGCTTGTGGCTCCACACACACAAAAAAAGACCTGTTAAAATTATACCTGTTGCTTAATTACAATA
TTTCTGATAACCATAGCATAGGACAAGGGAAAATAAAAAAAGAAAAAAAAGAAAAAAAAACGACAAATCTGTCTGCTG
GTCACTTCTTCTGTCCAAGCAGATTCGTGGTCTTTTCCTCGCTTCTTTCAAGGGCTTTCCTGTGCCAGGTGAAGGAGG
CTCCAGGCAGCACCCAGGTTTTGCACTCTTGTTTCTCCCGTGCTTGTGAAAGAGGTCCCAAGGTTCTGGGTGCAGGAG
CGCTCCCTTGACCTGCTGAAGTCCGGAACGTAGTCGGCACAGCCTGGTCGCCTTCCACCTCTGGGAGCTGGAGTCCAC
TGGGGTGGCCTGACTCCCCCAGTCCCCTTCCCGTGACCTGGTCAGGGTGAGCCCATGTGGAGTCAGCCTCGCAGGCCT
CCCTGCCAGTAGGGTCCGAGTGTGTTTCATCCTTCCCACTCTGTCGAGCCTGGGGGCTGGAGCGGAGACGGGAGGCCT
GGCCTGTCTCGGAACCTGTGAGCTGCACCAGGTAGAACGCCAGGGACCCCAGAATCATGTGCGTCAGTCCAAGGGGTC
CCCTCCAGGAGTAGTGAAGACTCCAGAAATGTCCCTTTCTTCTCCCCCATCCTACGAGTAATTGCATTTGCTTTTGTA
ATTCTTAATGAGCAATATCTGCTAGAGAGTTTAGCTGTAACAGTTCTTTTTGATCATCTTTTTTTAATAATTAGAAAC
ACCAAAAAAATCCAGAAACTTGTTCTTCCAAAGCAGAGAGCATTATAATCACCAGGGCCAAAAGCTTCCCTCCCTGCT
GTCATTGCTTCTTCTGAGGCCTGAATCCAAAAGAAAAACAGCCATAGGCCCTTTCAGTGGCCGGGCTACCCGTGAGCC
CTTCGGAGGACCAGGGCTGGGGCAGCCTCTGGGCCCACATCCGGGGCCAGCTCCGGCGTGTGTTCAGTGTTAGCAGTG
GGTCATGATGCTCTTTCCCACCCAGCCTGGGATAGGGGCAGAGGAGGCGAGGAGGCCGTTGCCGCTGATGTTTGGCCG
TGAACAGGTGGGTGTCTGCGTGCGTCCACGTGCGTGTTTTCTGACTGACATGAAATCGACGCCCGAGTTAGCCTCACC
CGGTGACCTCTAGCCCTGCCCGGATGGAGCGGGGCCCACCCGGTTCAGTGTTTCTGGGGAGCTGGACAGTGGAGTGCA
AAAGGCTTGCAGAACTTGAAGCCTGCTCCTTCCCTTGCTACCACGGCCTCCTTTCCGTTTGATTTGTCACTGCTTCAA
TCAATAACAGCCGCTCCAGAGTCAGTAGTCAATGAATATATGACCAAATATCACCAGGACTGTTACTCAATGTGTGCC
GAGCCCTTGCCCATGCTGGGCTCCCGTGTATCTGGACACTGTAACGTGTGCTGTGTTTGCTCCCCTTCCCCTTCCTTC
TTTGCCCTTTACTTGTCTTTCTGGGGTTTTTCTGTTTGGGTTTGGTTTGGTTTTTATTTCTCCTTTTGTGTTCCAAAC
ATGAGGTTCTCTCTACTGGTCCTCTTAACTGTGGTGTTGAGGCTTATATTTGTGTAATTTTTGGTGGGTGAAAGGAAT
TTTGCTAAGTAAATCTCTTCTGTGTTTGAACTGAAGTCTGTATTGTAACTATGTTTAAAGTAATTGTTCCAGAGACAA
ATATTTCTAGACACTTTTTCTTTACAAACAAAAGCATTCGGAGGGAGGGGGATGGTGACTGAGATGAGAGGGGAGAGC
TGAACAGATGACCCCTGCCCAGATCAGCCAGAAGCCACCCAAAGCAGTGGAGCCCAGGAGTCCCACTCCAAGCCAGCA
AGCCGAATAGCTGATGTGTTGCCACTTTCCAAGTCACTGCAAAACCAGGTTTTGTTCCGCCCAGTGGATTCTTGTTTT
GCTTCCCCTCCCCCCGAGATTATTACCACCATCCCGTGCTTTTAAGGAAAGGCAAGATTGATGTTTCCTTGAGGGGAG
CCAGGAGGGGATGTGTGTGTGCAGAGCTGAAGAGCTGGGGAGAATGGGGCTGGGCCCACCCAAGCAGGAGGCTGGGAC
GCTCTGCTGTGGGCACAGGTCAGGCTAATGTTGGCAGATGCAGCTCTTCCTGGACAGGCCAGGTGGTGGGCATTCTCT
CTCCAAGGTGTGCCCCGTGGGCATTACTGTTTAAGACACTTCCGTCACATCCCACCCCATCCTCCAGGGCTCAACACT
GTGACATCTCTATTCCCCACCCTCCCCTTCCCAGGGCAATAAAATGACCATGGAGGGGGCTTGCACTCTCTTGGCTGT
CACCCGATCGCCAGCAAAACTTAGATGTGAGAAAACCCCTTCCCATTCCATGGCGAAAACATCTCCTTAGAAAAGCCA
TTACCCTCATTAGGCATGGTTTTGGGCTCCCAAAACACCTGACAGCCCCTCCCTCCTCTGAGAGGCGGAGAGTGCTGA
CTGTAGTGACCATTGCATGCCGGGTGCAGCATCTGGAAGAGCTAGGCAGGGTGTCTGCCCCCTCCTGAGTTGAAGTCA
TGCTCCCCTGTGCCAGCCCAGAGGCCGAGAGCTATGGACAGCATTGCCAGTAACACAGGCCACCCTGTGCAGAAGGGA
GCTGGCTCCAGCCTGGAAACCTGTCTGAGGTTGGGAGAGGTGCACTTGGGGCACAGGGAGAGGCCGGGACACACTTAG
CTGGAGATGTCTCTAAAAGCCCTGTATCGTATTCACCTTCAGTTTTTGTGTTTTGGGACAATTACTTTAGAAAATAAG
TAGGTCGTTTTAAAAACAAAAATTATTGATTGCTTTTTTGTAGTGTTCAGAAAAAAGGTTCTTTGTGTATAGCCAAAT
GACTGAAAGCACTGATATATTTAAAAACAAAAGGCAATTTATTAAGGAAATTTGTACCATTTCAGTAAACCTGTCTGA
ATGTACCTGTATACGTTTCAAAAACACCCCCCCCCCACTGAATCCCTGTAACCTATTTATTATATAAAGAGTTTGCCT
TATAAATTTACATAAAAATGTCCGTTTGTGTCTTTTGTTGTAAAAATCAAGTGATTTTTTCATAAGGTTCTTTTACTA
TTGGAAAAGATGGGCAGCACGCAGTTTTATTTTATTTTTGTAAGTTTTTTAATACATGTGAAAGCAAAGAATACTCAG
CATGCCTTTCTAAGTGACGCGTTTGCACCTTTTGTTGGGAAGTACTGTATCCTGTGCTGTTAGCATTCTCGATAAATC
TCTCTGTGAAAGTGA
SEQ ID NO: 30 >NM_001386137.1 MECP2 [organism = Homo sapiens] [GeneID = 4204]
[transcript = 8]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGCACTGAGCTAGCCTCTCCTTGCTAGAAGCTTACGGTCGAAGGTCTCCAGCCATCAGAAGAAGCACGTGGAGCGC
TGCGTCCGTGTTGTGGTTATTCATCCAGCATGTGTTGAGGAAGAAAAGTCAGAAGACCAGGACCTCCAGGGCCTCAAG
GACAAACCCCTCAAGTTTAAAAAGGTGAAGAAAGATAAGAAAGAAGAGAAAGAGGGCAAGCATGAGCCCGTGCAGCCA
TCAGCCCACCACTCTGCTGAGCCCGCAGAGGCAGGCAAAGCAGAGACATCAGAAGGGTCAGGCTCCGCCCCGGCTGTG
CCGGAAGCTTCTGCCTCCCCCAAACAGCGGCGCTCCATCATCCGTGACCGGGGACCCATGTATGATGACCCCACCCTG
CCTGAAGGCTGGACACGGAAGCTTAAGCAAAGGAAATCTGGCCGCTCTGCTGGGAAGTATGATGTGTATTTGATCAAT
CCCCAGGGAAAAGCCTTTCGCTCTAAAGTGGAGTTGATTGCGTACTTCGAAAAGCTCCAGGAACTGGCAGAGGCCGGG
GACGCCCCAAAGGGAGCGGCACCACGAGACCCAAGGCGGCCACGTCAGAGGGTGTGCAGGTGAAAAGGGTCCTGGAGA
AAAGTCCTGGGAAGCTCCTTGTCAAGATGCCTTTTCAAACTTCGCCAGGGGGCAAGGCTGAGGGGGGTGGGGCCACCA
CATCCACCCAGGTCATGGTGATCAAACGCCCCGGCAGGAAGCGAAAAGCTGAGGCCGACCCTCAGGCCATTCCCAAGA
AACGGGGCCGAAAGCCGGGGAGTGTGGTGGCAGCCGCTGCCGCCGAGGCCAAAAAGAAAGCCGTGAAGGAGTCTTCTA
TCCGATCTGTGCAGGAGACCGTACTCCCCATCAAGAAGCGCAAGACCCGGGAGACGGTCAGCATCGAGGTCAAGGAAG
TGGTGAAGCCCCTGCTGGTGTCCACCCTCGGTGAGAAGAGCGGGAAAGGACTGAAGACCTGTAAGAGCCCTGGGCGGA
AAAGCAAGGAGAGCAGCCCCAAGGGGCGCAGCAGCAGCGCCTCCTCACCCCCCAAGAAGGAGCACCACCACCATCACC
ACCACTCAGAGTCCCCAAAGGCCCCCGTGCCACTGCTCCCACCCCTGCCCCCACCTCCACCTGAGCCCGAGAGCTCCG
AGGACCCCACCAGCCCCCCTGAGCCCCAGGACTTGAGCAGCAGCGTCTGCAAAGAGGAGAAGATGCCCAGAGGAGGCT
CACTGGAGAGCGACGGCTGCCCCAAGGAGCCAGCTAAGACTCAGCCCGCGGTTGCCACCGCCGCCACGGCCGCAGAAA
AGTACAAACACCGAGGGGAGGGAGAGCGCAAAGACATTGTTTCATCCTCCATGCCAAGGCCAAACAGAGAGGAGCCTG
TGGACAGCCGGACGCCCGTGACCGAGAGAGTTAGCTGACTTTACACGGAGCGGATTGCAAAGCAAACCAACAAGAATA
AAGGCAGCTGTTGTCTCTTCTCCTTATGGGTAGGGCTCTGACAAAGCTTCCCGATTAACTGAAATAAAAAATATTTTT
TTTTCTTTCAGTAAACTTAGAGTTTCGTGGCTTCAGGGTGGGAGTAGTTGGAGCATTGGGGATGTTTTTCTTACCGAC
AAGCACAGTCAGGTTGAAGACCTAACCAGGGCCAGAAGTAGCTTTGCACTTTTCTAAACTAGGCTCCTTCAACAAGGC
TTGCTGCAGATACTACTGACCAGACAAGCTGTTGACCAGGCACCTCCCCTCCCGCCCAAACCTTTCCCCCATGTGGTC
GTTAGAGACAGAGCGACAGAGCAGTTGAGAGGACACTCCCGTTTTCGGTGCCATCAGTGCCCCGTCTACAGCTCCCCC
AGCTCCCCCCACCTCCCCCACTCCCAACCACGTTGGGACAGGGAGGTGTGAGGCAGGAGAGACAGTTGGATTCTTTAG
AGAAGATGGATATGACCAGTGGCTATGGCCTGTGCGATCCCACCCGTGGTGGCTCAAGTCTGGCCCCACACCAGCCCC
AATCCAAAACTGGCAAGGACGCTTCACAGGACAGGAAAGTGGCACCTGTCTGCTCCAGCTCTGGCATGGCTAGGAGGG
GGGAGTCCCTTGAACTACTGGGTGTAGACTGGCCTGAACCACAGGAGAGGATGGCCCAGGGTGAGGTGGCATGGTCCA
TTCTCAAGGGACGTCCTCCAACGGGTGGCGCTAGAGGCCATGGAGGCAGTAGGACAAGGTGCAGGCAGGCTGGCCTGG
GGTCAGGCCGGGCAGAGCACAGCGGGGTGAGAGGGATTCCTAATCACTCAGAGCAGTCTGTGACTTAGTGGACAGGGG
AGGGGGCAAAGGGGGAGGAGAAGAAAATGTTCTTCCAGTTACTTTCCAATTCTCCTTTAGGGACAGCTTAGAATTATT
TGCACTATTGAGTCTTCATGTTCCCACTTCAAAACAAACAGATGCTCTGAGAGCAAACTGGCTTGAATTGGTGACATT
TAGTCCCTCAAGCCACCAGATGTGACAGTGTTGAGAACTACCTGGATTTGTATATATACCTGCGCTTGTTTTAAAGTG
GGCTCAGCACATAGGGTTCCCACGAAGCTCCGAAACTCTAAGTGTTTGCTGCAATTTTATAAGGACTTCCTGATTGGT
TTCTCTTCTCCCCTTCCATTTCTGCCTTTTGTTCATTTCATCCTTTCACTTCTTTCCCTTCCTCCATCCTCCTCCTTC
CTAGTTCATCCCTTCTCTTCCAGGCAGCCGCGGTGCCCAACCACACTTGTCGGCTCCAGTCCCCAGAACTCTGCCTGC
CCTTTGTCCTCCTGCTGCCAGTACCAGCCCCACCCTGTTTTGAGCCCTGAGGAGGCCTTGGGCTCTGCTGAGTCCGAC
CTGGCCTGTCTGTGAAGAGCAAGAGAGCAGCAAGGTCTTGCTCTCCTAGGTAGCCCCCTCTTCCCTGGTAAGAAAAAG
CAAAAGGCATTTCCCACCCTGAACAACGAGCCTTTTCACCCTTCTACTCTAGAGAAGTGGACTGGAGGAGCTGGGCCC
GATTTGGTAGTTGAGGAAAGCACAGAGGCCTCCTGTGGCCTGCCAGTCATCGAGTGGCCCAACAGGGGCTCCATGCCA
GCCGACCTTGACCTCACTCAGAAGTCCAGAGTCTAGCGTAGTGCAGCAGGGCAGTAGCGGTACCAATGCAGAACTCCC
AAGACCCGAGCTGGGACCAGTACCTGGGTCCCCAGCCCTTCCTCTGCTCCCCCTTTTCCCTCGGAGTTCTTCTTGAAT
GGCAATGTTTTGCTTTTGCTCGATGCAGACAGGGGGCCAGAACACCACACATTTCACTGTCTGTCTGGTCCATAGCTG
TGGTGTAGGGGCTTAGAGGCATGGGCTTGCTGTGGGTTTTTAATTGATCAGTTTTCATGTGGGATCCCATCTTTTTAA
CCTCTGTTCAGGAAGTCCTTATCTAGCTGCATATCTTCATCATATTGGTATATCCTTTTCTGTGTTTACAGAGATGTC
TCTTATATCTAAATCTGTCCAACTGAGAAGTACCTTATCAAAGTAGCAAATGAGACAGCAGTCTTATGCTTCCAGAAA
CACCCACAGGCATGTCCCATGTGAGCTGCTGCCATGAACTGTCAAGTGTGTGTTGTCTTGTGTATTTCAGTTATTGTC
CCTGGCTTCCTTACTATGGTGTAATCATGAAGGAGTGAAACATCATAGAAACTGTCTAGCACTTCCTTGCCAGTCTTT
AGTGATCAGGAACCATAGTTGACAGTTCCAATCAGTAGCTTAAGAAAAAACCGTGTTTGTCTCTTCTGGAATGGTTAG
AAGTGAGGGAGTTTGCCCCGTTCTGTTTGTAGAGTCTCATAGTTGGACTTTCTAGCATATATGTGTCCATTTCCTTAT
GCTGTAAAAGCAAGTCCTGCAACCAAACTCCCATCAGCCCAATCCCTGATCCCTGATCCCTTCCACCTGCTCTGCTGA
TGACCCCCCCAGCTTCACTTCTGACTCTTCCCCAGGAAGGGAAGGGGGGTCAGAAGAGAGGGTGAGTCCTCCAGAACT
CTTCCTCCAAGGACAGAAGGCTCCTGCCCCCATAGTGGCCTCGAACTCCTGGCACTACCAAAGGACACTTATCCACGA
GAGCGCAGCATCCGACCAGGTTGTCACTGAGAAGATGTTTATTTTGGTCAGTTGGGTTTTTATGTATTATACTTAGTC
AAATGTAATGTGGCTTCTGGAATCATTGTCCAGAGCTGCTTCCCCGTCACCTGGGCGTCATCTGGTCCTGGTAAGAGG
AGTGCGTGGCCCACCAGGCCCCCCTGTCACCCATGACAGTTCATTCAGGGCCGATGGGGCAGTCGTGGTTGGGAACAC
AGCATTTCAAGCGTCACTTTATTTCATTCGGGCCCCACCTGCAGCTCCCTCAAAGAGGCAGTTGCCCAGCCTCTTTCC
CTTCCAGTTTATTCCAGAGCTGCCAGTGGGGCCTGAGGCTCCTTAGGGTTTTCTCTCTATTTCCCCCTTTCTTCCTCA
TTCCCTCGTCTTTCCCAAAGGCATCACGAGTCAGTCGCCTTTCAGCAGGCAGCCTTGGCGGTTTATCGCCCTGGCAGG
CAGGGGCCCTGCAGCTCTCATGCTGCCCCTGCCTTGGGGTCAGGTTGACAGGAGGTTGGAGGGAAAGCCTTAAGCTGC
AGGATTCTCACCAGCTGTGTCCGGCCCAGTTTTGGGGTGTGACCTCAATTTCAATTTTGTCTGTACTTGAACATTATG
AAGATGGGGGCCTCTTTCAGTGAATTTGTGAACAGCAGAATTGACCGACAGCTTTCCAGTACCCATGGGGCTAGGTCA
TTAAGGCCACATCCACAGTCTCCCCCACCCTTGTTCCAGTTGTTAGTTACTACCTCCTCTCCTGACAATACTGTATGT
CGTCGAGCTCCCCCCAGGTCTACCCCTCCCGGCCCTGCCTGCTGGTGGGCTTGTCATAGCCAGTGGGATTGCCGGTCT
TGACAGCTCAGTGAGCTGGAGATACTTGGTCACAGCCAGGCGCTAGCACAGCTCCCTTCTGTTGATGCTGTATTCCCA
TATCAAAAGACACAGGGGACACCCAGAAACGCCACATCCCCCAATCCATCAGTGCCAAACTAGCCAACGGCCCCAGCT
TCTCAGCTCGCTGGATGGCGGAAGCTGCTACTCGTGAGCGCCAGTGCGGGTGCAGACAATCTTCTGTTGGGTGGCATC
ATTCCAGGCCCGAAGCATGAACAGTGCACCTGGGACAGGGAGCAGCCCCAAATTGTCACCTGCTTCTCTGCCCAGCTT
TTCATTGCTGTGACAGTGATGGCGAAAGAGGGTAATAACCAGACACAAACTGCCAAGTTGGGTGGAGAAAGGAGTTTC
TTTAGCTGACAGAATCTCTGAATTTTAAATCACTTAGTAAGCGGCTCAAGCCCAGGAGGGAGCAGAGGGATACGAGCG
GAGTCCCCTGCGCGGGACCATCTGGAATTGGTTTAGCCCAAGTGGAGCCTGACAGCCAGAACTCTGTGTCCCCCGTCT
AACCACAGCTCCTTTTCCAGAGCATTCCAGTCAGGCTCTCTGGGCTGACTGGGCCAGGGGAGGTTACAGGTACCAGTT
CTTTAAGAAGATCTTTGGGCATATACATTTTTAGCCTGTGTCATTGCCCCAAATGGATTCCTGTTTCAAGTTCACACC
TGCAGATTCTAGGACCTGTGTCCTAGACTTCAGGGAGTCAGCTGTTTCTAGAGTTCCTACCATGGAGTGGGTCTGGAG
GACCTGCCCGGTGGGGGGGCAGAGCCCTGCTCCCTCCGGGTCTTCCTACTCTTCTCTCTGCTCTGACGGGATTTGTTG
ATTCTCTCCATTTTGGTGTCTTTCTCTTTTAGATATTGTATCAATCTTTAGAAAAGGCATAGTCTACTTGTTATAAAT
CGTTAGGATACTGCCTCCCCCAGGGTCTAAAATTACATATTAGAGGGGAAAAGCTGAACACTGAAGTCAGTTCTCAAC
AATTTAGAAGGAAAACCTAGAAAACATTTGGCAGAAAATTACATTTCGATGTTTTTGAATGAATACGAGCAAGCTTTT
ACAACAGTGCTGATCTAAAAATACTTAGCACTTGGCCTGAGATGCCTGGTGAGCATTACAGGCAAGGGGAATCTGGAG
GTAGCCGACCTGAGGACATGGCTTCTGAACCTGTCTTTTGGGAGTGGTATGGAAGGTGGAGCGTTCACCAGTGACCTG
GAAGGCCCAGCACCACCCTCCTTCCCACTCTTCTCATCTTGACAGAGCCTGCCCCAGCGCTGACGTGTCAGGAAAACA
CCCAGGGAACTAGGAAGGCACTTCTGCCTGAGGGGCAGCCTGCCTTGCCCACTCCTGCTCTGCTCGCCTCGGATCAGC
TGAGCCTTCTGAGCTGGCCTCTCACTGCCTCCCCAAGGCCCCCTGCCTGCCCTGTCAGGAGGCAGAAGGAAGCAGGTG
TGAGGGCAGTGCAAGGAGGGAGCACAACCCCCAGCTCCCGCTCCGGGCTCCGACTTGTGCACAGGCAGAGCCCAGACC
CTGGAGGAAATCCTACCTTTGAATTCAAGAACATTTGGGGAATTTGGAAATCTCTTTGCCCCCAAACCCCCATTCTGT
CCTACCTTTAATCAGGTCCTGCTCAGCAGTGAGAGCAGATGAGGTGAAAAGGCCAAGAGGTTTGGCTCCTGCCCACTG
ATAGCCCCTCTCCCCGCAGTGTTTGTGTGTCAAGTGGCAAAGCTGTTCTTCCTGGTGACCCTGATTATATCCAGTAAC
ACATAGACTGTGCGCATAGGCCTGCTTTGTCTCCTCTATCCTGGGCTTTTGTTTTGCTTTTTAGTTTTGCTTTTAGTT
TTTCTGTCCCTTTTATTTAACGCACCGACTAGACACACAAAGCAGTTGAATTTTTATATATATATCTGTATATTGCAC
AATTATAAACTCATTTTGCTTGTGGCTCCACACACACAAAAAAAGACCTGTTAAAATTATACCTGTTGCTTAATTACA
ATATTTCTGATAACCATAGCATAGGACAAGGGAAAATAAAAAAAGAAAAAAAAGAAAAAAAAACGACAAATCTGTCTG
CTGGTCACTTCTTCTGTCCAAGCAGATTCGTGGTCTTTTCCTCGCTTCTTTCAAGGGCTTTCCTGTGCCAGGTGAAGG
AGGCTCCAGGCAGCACCCAGGTTTTGCACTCTTGTTTCTCCCGTGCTTGTGAAAGAGGTCCCAAGGTTCTGGGTGCAG
GAGCGCTCCCTTGACCTGCTGAAGTCCGGAACGTAGTCGGCACAGCCTGGTCGCCTTCCACCTCTGGGAGCTGGAGTC
CACTGGGGTGGCCTGACTCCCCCAGTCCCCTTCCCGTGACCTGGTCAGGGTGAGCCCATGTGGAGTCAGCCTCGCAGG
CCTCCCTGCCAGTAGGGTCCGAGTGTGTTTCATCCTTCCCACTCTGTCGAGCCTGGGGGCTGGAGCGGAGACGGGAGG
CCTGGCCTGTCTCGGAACCTGTGAGCTGCACCAGGTAGAACGCCAGGGACCCCAGAATCATGTGCGTCAGTCCAAGGG
GTCCCCTCCAGGAGTAGTGAAGACTCCAGAAATGTCCCTTTCTTCTCCCCCATCCTACGAGTAATTGCATTTGCTTTT
GTAATTCTTAATGAGCAATATCTGCTAGAGAGTTTAGCTGTAACAGTTCTTTTTGATCATCTTTTTTTAATAATTAGA
AACACCAAAAAAATCCAGAAACTTGTTCTTCCAAAGCAGAGAGCATTATAATCACCAGGGCCAAAAGCTTCCCTCCCT
GCTGTCATTGCTTCTTCTGAGGCCTGAATCCAAAAGAAAAACAGCCATAGGCCCTTTCAGTGGCCGGGCTACCCGTGA
GCCCTTCGGAGGACCAGGGCTGGGGCAGCCTCTGGGCCCACATCCGGGGCCAGCTCCGGCGTGTGTTCAGTGTTAGCA
GTGGGTCATGATGCTCTTTCCCACCCAGCCTGGGATAGGGGCAGAGGAGGCGAGGAGGCCGTTGCCGCTGATGTTTGG
CCGTGAACAGGTGGGTGTCTGCGTGCGTCCACGTGCGTGTTTTCTGACTGACATGAAATCGACGCCCGAGTTAGCCTC
ACCCGGTGACCTCTAGCCCTGCCCGGATGGAGCGGGGCCCACCCGGTTCAGTGTTTCTGGGGAGCTGGACAGTGGAGT
GCAAAAGGCTTGCAGAACTTGAAGCCTGCTCCTTCCCTTGCTACCACGGCCTCCTTTCCGTTTGATTTGTCACTGCTT
CAATCAATAACAGCCGCTCCAGAGTCAGTAGTCAATGAATATATGACCAAATATCACCAGGACTGTTACTCAATGTGT
GCCGAGCCCTTGCCCATGCTGGGCTCCCGTGTATCTGGACACTGTAACGTGTGCTGTGTTTGCTCCCCTTCCCCTTCC
TTCTTTGCCCTTTACTTGTCTTTCTGGGGTTTTTCTGTTTGGGTTTGGTTTGGTTTTTATTTCTCCTTTTGTGTTCCA
AACATGAGGTTCTCTCTACTGGTCCTCTTAACTGTGGTGTTGAGGCTTATATTTGTGTAATTTTTGGTGGGTGAAAGG
AATTTTGCTAAGTAAATCTCTTCTGTGTTTGAACTGAAGTCTGTATTGTAACTATGTTTAAAGTAATTGTTCCAGAGA
CAAATATTTCTAGACACTTTTTCTTTACAAACAAAAGCATTCGGAGGGAGGGGGATGGTGACTGAGATGAGAGGGGAG
AGCTGAACAGATGACCCCTGCCCAGATCAGCCAGAAGCCACCCAAAGCAGTGGAGCCCAGGAGTCCCACTCCAAGCCA
GCAAGCCGAATAGCTGATGTGTTGCCACTTTCCAAGTCACTGCAAAACCAGGTTTTGTTCCGCCCAGTGGATTCTTGT
TTTGCTTCCCCTCCCCCCGAGATTATTACCACCATCCCGTGCTTTTAAGGAAAGGCAAGATTGATGTTTCCTTGAGGG
GAGCCAGGAGGGGATGTGTGTGTGCAGAGCTGAAGAGCTGGGGAGAATGGGGCTGGGCCCACCCAAGCAGGAGGCTGG
GACGCTCTGCTGTGGGCACAGGTCAGGCTAATGTTGGCAGATGCAGCTCTTCCTGGACAGGCCAGGTGGTGGGCATTC
TCTCTCCAAGGTGTGCCCCGTGGGCATTACTGTTTAAGACACTTCCGTCACATCCCACCCCATCCTCCAGGGCTCAAC
ACTGTGACATCTCTATTCCCCACCCTCCCCTTCCCAGGGCAATAAAATGACCATGGAGGGGGCTTGCACTCTCTTGGC
TGTCACCCGATCGCCAGCAAAACTTAGATGTGAGAAAACCCCTTCCCATTCCATGGCGAAAACATCTCCTTAGAAAAG
CCATTACCCTCATTAGGCATGGTTTTGGGCTCCCAAAACACCTGACAGCCCCTCCCTCCTCTGAGAGGCGGAGAGTGC
TGACTGTAGTGACCATTGCATGCCGGGTGCAGCATCTGGAAGAGCTAGGCAGGGTGTCTGCCCCCTCCTGAGTTGAAG
TCATGCTCCCCTGTGCCAGCCCAGAGGCCGAGAGCTATGGACAGCATTGCCAGTAACACAGGCCACCCTGTGCAGAAG
GGAGCTGGCTCCAGCCTGGAAACCTGTCTGAGGTTGGGAGAGGTGCACTTGGGGCACAGGGAGAGGCCGGGACACACT
TAGCTGGAGATGTCTCTAAAAGCCCTGTATCGTATTCACCTTCAGTTTTTGTGTTTTGGGACAATTACTTTAGAAAAT
AAGTAGGTCGTTTTAAAAACAAAAATTATTGATTGCTTTTTTGTAGTGTTCAGAAAAAAGGTTCTTTGTGTATAGCCA
AATGACTGAAAGCACTGATATATTTAAAAACAAAAGGCAATTTATTAAGGAAATTTGTACCATTTCAGTAAACCTGTC
TGAATGTACCTGTATACGTTTCAAAAACACCCCCCCCCCACTGAATCCCTGTAACCTATTTATTATATAAAGAGTTTG
CCTTATAAATTTACATAAAAATGTCCGTTTGTGTCTTTTGTTGTAAAAATCAAGTGATTTTTTCATAAGGTTCTTTTA
CTATTGGAAAAGATGGGCAGCACGCAGTTTTATTTTATTTTTGTAAGTTTTTTAATACATGTGAAAGCAAAGAATACT
CAGCATGCCTTTCTAAGTGACGCGTTTGCACCTTTTGTTGGGAAGTACTGTATCCTGTGCTGTTAGCATTCTCGATAA
ATCTCTCTGTGAAAGTGA
SEQ ID NO: 31 >NM_001369392.2 MECP2 [organism = Homo sapiens] [GeneID = 4204]
[transcript = 5]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGCACTGAGCTAGCCTCTCCTTGCTAGAAGCTTACGGTCGAAGGTCTCCAGCCATCAGAAGAAGCACGTGGAGCGC
TGCGTCCGTGTTGTGGTTATTCATCCAGCATGTGTTGAGGAAGAAAAGTCAGAAGACCAGGACCTCCAGGGCCTCAAG
GACAAACCCCTCAAGTTTAAAAAGGTGAAGAAAGATAAGAAAGAAGAGAAAGAGGGCAAGCATGAGCCCGTGCAGCCA
TCAGCCCACCACTCTGCTGAGCCCGCAGAGGCAGGCAAAGCAGAGACATCAGAAGGGTCAGGCTCCGCCCCGGCTGTG
CCGGAAGCTTCTGCCTCCCCCAAACAGCGGCGCTCCATCATCCGTGACCGGGGACCCATGTATGATGACCCCACCCTG
CCTGAAGGCTGGACACGGAAGCTTAAGCAAAGGAAATCTGGCCGCTCTGCTGGGAAGTATGATGTGTATTTGATCAAT
CCCCAGGGAAAAGCCTTTCGCTCTAAAGTGGAGTTGATTGCGTACTTCGAAAAGGTAGGCGACACATCCCTGGACCCT
AATGATTTTGACTTCACGGTAACTGGGAGAGGGAGCCCCTCCCGGCGAGAGCAGAAACCACCTAAGAAGCCCAAATCT
CCCAAAGCTCCAGGAACTGGCAGAGGCCGGGGACGCCCCAAAGGGAGCGGCACCACGAGACCCAAGGCGGCCACGTCA
GAGGGTGTGCAGGTGAAAAGGGTCCTGGAGAAAAGTCCTGGGAAGCTCCTTGTCAAGATGCCTTTTCAAACTTCGCCA
GGGGGCAAGGCTGAGGGGGGTGGGGCCACCACATCCACCCAGGTCATGGTGATCAAACGCCCCGGCAGGAAGCGAAAA
GCTGAGGCCGACCCTCAGGCCATTCCCAAGAAACGGGGCCGAAAGCCGGGGAGTGTGGTGGCAGCCGCTGCCGCCGAG
GCCAAAAAGAAAGCCGTGAAGGAGTCTTCTATCCGATCTGTGCAGGAGACCGTACTCCCCATCAAGAAGCGCAAGACC
CGGGAGACGGTCAGCATCGAGGTCAAGGAAGTGGTGAAGCCCCTGCTGGTGTCCACCCTCGGTGAGAAGAGCGGGAAA
GGACTGAAGACCTGTAAGAGCCCTGGGCGGAAAAGCAAGGAGAGCAGCCCCAAGGGGCGCAGCAGCAGCGCCTCCTCA
CCCCCCAAGAAGGAGCACCACCACCATCACCACCACTCAGAGTCCCCAAAGGCCCCCGTGCCACTGCTCCCACCCCTG
CCCCCACCTCCACCTGAGCCCGAGAGCTCCGAGGACCCCACCAGCCCCCCTGAGCCCCAGGACTTGAGCAGCAGCGTC
TGCAAAGAGGAGAAGATGCCCAGAGGAGGCTCACTGGAGAGCGACGGCTGCCCCAAGGAGCCAGCTAAGACTCAGCCC
GCGGTTGCCACCGCCGCCACGGCCGCAGAAAAGTACAAACACCGAGGGGAGGGAGAGCGCAAAGACATTGTTTCATCC
TCCATGCCAAGGCCAAACAGAGAGGAGCCTGTGGACAGCCGGACGCCCGTGACCGAGAGAGTTAGCTGACTTTACACG
GAGCGGATTGCAAAGCAAACCAACAAGAATAAAGGCAGCTGTTGTCTCTTCTCCTTATGGGTAGGGCTCTGACAAAGC
TTCCCGATTAACTGAAATAAAAAATATTTTTTTTTCTTTCAGTAAACTTAGAGTTTCGTGGCTTCAGGGTGGGAGTAG
TTGGAGCATTGGGGATGTTTTTCTTACCGACAAGCACAGTCAGGTTGAAGACCTAACCAGGGCCAGAAGTAGCTTTGC
ACTTTTCTAAACTAGGCTCCTTCAACAAGGCTTGCTGCAGATACTACTGACCAGACAAGCTGTTGACCAGGCACCTCC
CCTCCCGCCCAAACCTTTCCCCCATGTGGTCGTTAGAGACAGAGCGACAGAGCAGTTGAGAGGACACTCCCGTTTTCG
GTGCCATCAGTGCCCCGTCTACAGCTCCCCCAGCTCCCCCCACCTCCCCCACTCCCAACCACGTTGGGACAGGGAGGT
GTGAGGCAGGAGAGACAGTTGGATTCTTTAGAGAAGATGGATATGACCAGTGGCTATGGCCTGTGCGATCCCACCCGT
GGTGGCTCAAGTCTGGCCCCACACCAGCCCCAATCCAAAACTGGCAAGGACGCTTCACAGGACAGGAAAGTGGCACCT
GTCTGCTCCAGCTCTGGCATGGCTAGGAGGGGGGAGTCCCTTGAACTACTGGGTGTAGACTGGCCTGAACCACAGGAG
AGGATGGCCCAGGGTGAGGTGGCATGGTCCATTCTCAAGGGACGTCCTCCAACGGGTGGCGCTAGAGGCCATGGAGGC
AGTAGGACAAGGTGCAGGCAGGCTGGCCTGGGGTCAGGCCGGGCAGAGCACAGCGGGGTGAGAGGGATTCCTAATCAC
TCAGAGCAGTCTGTGACTTAGTGGACAGGGGAGGGGGCAAAGGGGGAGGAGAAGAAAATGTTCTTCCAGTTACTTTCC
AATTCTCCTTTAGGGACAGCTTAGAATTATTTGCACTATTGAGTCTTCATGTTCCCACTTCAAAACAAACAGATGCTC
TGAGAGCAAACTGGCTTGAATTGGTGACATTTAGTCCCTCAAGCCACCAGATGTGACAGTGTTGAGAACTACCTGGAT
TTGTATATATACCTGCGCTTGTTTTAAAGTGGGCTCAGCACATAGGGTTCCCACGAAGCTCCGAAACTCTAAGTGTTT
GCTGCAATTTTATAAGGACTTCCTGATTGGTTTCTCTTCTCCCCTTCCATTTCTGCCTTTTGTTCATTTCATCCTTTC
ACTTCTTTCCCTTCCTCCATCCTCCTCCTTCCTAGTTCATCCCTTCTCTTCCAGGCAGCCGCGGTGCCCAACCACACT
TGTCGGCTCCAGTCCCCAGAACTCTGCCTGCCCTTTGTCCTCCTGCTGCCAGTACCAGCCCCACCCTGTTTTGAGCCC
TGAGGAGGCCTTGGGCTCTGCTGAGTCCGACCTGGCCTGTCTGTGAAGAGCAAGAGAGCAGCAAGGTCTTGCTCTCCT
AGGTAGCCCCCTCTTCCCTGGTAAGAAAAAGCAAAAGGCATTTCCCACCCTGAACAACGAGCCTTTTCACCCTTCTAC
TCTAGAGAAGTGGACTGGAGGAGCTGGGCCCGATTTGGTAGTTGAGGAAAGCACAGAGGCCTCCTGTGGCCTGCCAGT
CATCGAGTGGCCCAACAGGGGCTCCATGCCAGCCGACCTTGACCTCACTCAGAAGTCCAGAGTCTAGCGTAGTGCAGC
AGGGCAGTAGCGGTACCAATGCAGAACTCCCAAGACCCGAGCTGGGACCAGTACCTGGGTCCCCAGCCCTTCCTCTGC
TCCCCCTTTTCCCTCGGAGTTCTTCTTGAATGGCAATGTTTTGCTTTTGCTCGATGCAGACAGGGGGCCAGAACACCA
CACATTTCACTGTCTGTCTGGTCCATAGCTGTGGTGTAGGGGCTTAGAGGCATGGGCTTGCTGTGGGTTTTTAATTGA
TCAGTTTTCATGTGGGATCCCATCTTTTTAACCTCTGTTCAGGAAGTCCTTATCTAGCTGCATATCTTCATCATATTG
GTATATCCTTTTCTGTGTTTACAGAGATGTCTCTTATATCTAAATCTGTCCAACTGAGAAGTACCTTATCAAAGTAGC
AAATGAGACAGCAGTCTTATGCTTCCAGAAACACCCACAGGCATGTCCCATGTGAGCTGCTGCCATGAACTGTCAAGT
GTGTGTTGTCTTGTGTATTTCAGTTATTGTCCCTGGCTTCCTTACTATGGTGTAATCATGAAGGAGTGAAACATCATA
GAAACTGTCTAGCACTTCCTTGCCAGTCTTTAGTGATCAGGAACCATAGTTGACAGTTCCAATCAGTAGCTTAAGAAA
AAACCGTGTTTGTCTCTTCTGGAATGGTTAGAAGTGAGGGAGTTTGCCCCGTTCTGTTTGTAGAGTCTCATAGTTGGA
CTTTCTAGCATATATGTGTCCATTTCCTTATGCTGTAAAAGCAAGTCCTGCAACCAAACTCCCATCAGCCCAATCCCT
GATCCCTGATCCCTTCCACCTGCTCTGCTGATGACCCCCCCAGCTTCACTTCTGACTCTTCCCCAGGAAGGGAAGGGG
GGTCAGAAGAGAGGGTGAGTCCTCCAGAACTCTTCCTCCAAGGACAGAAGGCTCCTGCCCCCATAGTGGCCTCGAACT
CCTGGCACTACCAAAGGACACTTATCCACGAGAGCGCAGCATCCGACCAGGTTGTCACTGAGAAGATGTTTATTTTGG
TCAGTTGGGTTTTTATGTATTATACTTAGTCAAATGTAATGTGGCTTCTGGAATCATTGTCCAGAGCTGCTTCCCCGT
CACCTGGGCGTCATCTGGTCCTGGTAAGAGGAGTGCGTGGCCCACCAGGCCCCCCTGTCACCCATGACAGTTCATTCA
GGGCCGATGGGGCAGTCGTGGTTGGGAACACAGCATTTCAAGCGTCACTTTATTTCATTCGGGCCCCACCTGCAGCTC
CCTCAAAGAGGCAGTTGCCCAGCCTCTTTCCCTTCCAGTTTATTCCAGAGCTGCCAGTGGGGCCTGAGGCTCCTTAGG
GTTTTCTCTCTATTTCCCCCTTTCTTCCTCATTCCCTCGTCTTTCCCAAAGGCATCACGAGTCAGTCGCCTTTCAGCA
GGCAGCCTTGGCGGTTTATCGCCCTGGCAGGCAGGGGCCCTGCAGCTCTCATGCTGCCCCTGCCTTGGGGTCAGGTTG
ACAGGAGGTTGGAGGGAAAGCCTTAAGCTGCAGGATTCTCACCAGCTGTGTCCGGCCCAGTTTTGGGGTGTGACCTCA
ATTTCAATTTTGTCTGTACTTGAACATTATGAAGATGGGGGCCTCTTTCAGTGAATTTGTGAACAGCAGAATTGACCG
ACAGCTTTCCAGTACCCATGGGGCTAGGTCATTAAGGCCACATCCACAGTCTCCCCCACCCTTGTTCCAGTTGTTAGT
TACTACCTCCTCTCCTGACAATACTGTATGTCGTCGAGCTCCCCCCAGGTCTACCCCTCCCGGCCCTGCCTGCTGGTG
GGCTTGTCATAGCCAGTGGGATTGCCGGTCTTGACAGCTCAGTGAGCTGGAGATACTTGGTCACAGCCAGGCGCTAGC
ACAGCTCCCTTCTGTTGATGCTGTATTCCCATATCAAAAGACACAGGGGACACCCAGAAACGCCACATCCCCCAATCC
ATCAGTGCCAAACTAGCCAACGGCCCCAGCTTCTCAGCTCGCTGGATGGCGGAAGCTGCTACTCGTGAGCGCCAGTGC
GGGTGCAGACAATCTTCTGTTGGGTGGCATCATTCCAGGCCCGAAGCATGAACAGTGCACCTGGGACAGGGAGCAGCC
CCAAATTGTCACCTGCTTCTCTGCCCAGCTTTTCATTGCTGTGACAGTGATGGCGAAAGAGGGTAATAACCAGACACA
AACTGCCAAGTTGGGTGGAGAAAGGAGTTTCTTTAGCTGACAGAATCTCTGAATTTTAAATCACTTAGTAAGCGGCTC
AAGCCCAGGAGGGAGCAGAGGGATACGAGCGGAGTCCCCTGCGCGGGACCATCTGGAATTGGTTTAGCCCAAGTGGAG
CCTGACAGCCAGAACTCTGTGTCCCCCGTCTAACCACAGCTCCTTTTCCAGAGCATTCCAGTCAGGCTCTCTGGGCTG
ACTGGGCCAGGGGAGGTTACAGGTACCAGTTCTTTAAGAAGATCTTTGGGCATATACATTTTTAGCCTGTGTCATTGC
CCCAAATGGATTCCTGTTTCAAGTTCACACCTGCAGATTCTAGGACCTGTGTCCTAGACTTCAGGGAGTCAGCTGTTT
CTAGAGTTCCTACCATGGAGTGGGTCTGGAGGACCTGCCCGGTGGGGGGGCAGAGCCCTGCTCCCTCCGGGTCTTCCT
ACTCTTCTCTCTGCTCTGACGGGATTTGTTGATTCTCTCCATTTTGGTGTCTTTCTCTTTTAGATATTGTATCAATCT
TTAGAAAAGGCATAGTCTACTTGTTATAAATCGTTAGGATACTGCCTCCCCCAGGGTCTAAAATTACATATTAGAGGG
GAAAAGCTGAACACTGAAGTCAGTTCTCAACAATTTAGAAGGAAAACCTAGAAAACATTTGGCAGAAAATTACATTTC
GATGTTTTTGAATGAATACGAGCAAGCTTTTACAACAGTGCTGATCTAAAAATACTTAGCACTTGGCCTGAGATGCCT
GGTGAGCATTACAGGCAAGGGGAATCTGGAGGTAGCCGACCTGAGGACATGGCTTCTGAACCTGTCTTTTGGGAGTGG
TATGGAAGGTGGAGCGTTCACCAGTGACCTGGAAGGCCCAGCACCACCCTCCTTCCCACTCTTCTCATCTTGACAGAG
CCTGCCCCAGCGCTGACGTGTCAGGAAAACACCCAGGGAACTAGGAAGGCACTTCTGCCTGAGGGGCAGCCTGCCTTG
CCCACTCCTGCTCTGCTCGCCTCGGATCAGCTGAGCCTTCTGAGCTGGCCTCTCACTGCCTCCCCAAGGCCCCCTGCC
TGCCCTGTCAGGAGGCAGAAGGAAGCAGGTGTGAGGGCAGTGCAAGGAGGGAGCACAACCCCCAGCTCCCGCTCCGGG
CTCCGACTTGTGCACAGGCAGAGCCCAGACCCTGGAGGAAATCCTACCTTTGAATTCAAGAACATTTGGGGAATTTGG
AAATCTCTTTGCCCCCAAACCCCCATTCTGTCCTACCTTTAATCAGGTCCTGCTCAGCAGTGAGAGCAGATGAGGTGA
AAAGGCCAAGAGGTTTGGCTCCTGCCCACTGATAGCCCCTCTCCCCGCAGTGTTTGTGTGTCAAGTGGCAAAGCTGTT
CTTCCTGGTGACCCTGATTATATCCAGTAACACATAGACTGTGCGCATAGGCCTGCTTTGTCTCCTCTATCCTGGGCT
TTTGTTTTGCTTTTTAGTTTTGCTTTTAGTTTTTCTGTCCCTTTTATTTAACGCACCGACTAGACACACAAAGCAGTT
GAATTTTTATATATATATCTGTATATTGCACAATTATAAACTCATTTTGCTTGTGGCTCCACACACACAAAAAAAGAC
CTGTTAAAATTATACCTGTTGCTTAATTACAATATTTCTGATAACCATAGCATAGGACAAGGGAAAATAAAAAAAGAA
AAAAAAGAAAAAAAAACGACAAATCTGTCTGCTGGTCACTTCTTCTGTCCAAGCAGATTCGTGGTCTTTTCCTCGCTT
CTTTCAAGGGCTTTCCTGTGCCAGGTGAAGGAGGCTCCAGGCAGCACCCAGGTTTTGCACTCTTGTTTCTCCCGTGCT
TGTGAAAGAGGTCCCAAGGTTCTGGGTGCAGGAGCGCTCCCTTGACCTGCTGAAGTCCGGAACGTAGTCGGCACAGCC
TGGTCGCCTTCCACCTCTGGGAGCTGGAGTCCACTGGGGTGGCCTGACTCCCCCAGTCCCCTTCCCGTGACCTGGTCA
GGGTGAGCCCATGTGGAGTCAGCCTCGCAGGCCTCCCTGCCAGTAGGGTCCGAGTGTGTTTCATCCTTCCCACTCTGT
CGAGCCTGGGGGCTGGAGCGGAGACGGGAGGCCTGGCCTGTCTCGGAACCTGTGAGCTGCACCAGGTAGAACGCCAGG
GACCCCAGAATCATGTGCGTCAGTCCAAGGGGTCCCCTCCAGGAGTAGTGAAGACTCCAGAAATGTCCCTTTCTTCTC
CCCCATCCTACGAGTAATTGCATTTGCTTTTGTAATTCTTAATGAGCAATATCTGCTAGAGAGTTTAGCTGTAACAGT
TCTTTTTGATCATCTTTTTTTAATAATTAGAAACACCAAAAAAATCCAGAAACTTGTTCTTCCAAAGCAGAGAGCATT
ATAATCACCAGGGCCAAAAGCTTCCCTCCCTGCTGTCATTGCTTCTTCTGAGGCCTGAATCCAAAAGAAAAACAGCCA
TAGGCCCTTTCAGTGGCCGGGCTACCCGTGAGCCCTTCGGAGGACCAGGGCTGGGGCAGCCTCTGGGCCCACATCCGG
GGCCAGCTCCGGCGTGTGTTCAGTGTTAGCAGTGGGTCATGATGCTCTTTCCCACCCAGCCTGGGATAGGGGCAGAGG
AGGCGAGGAGGCCGTTGCCGCTGATGTTTGGCCGTGAACAGGTGGGTGTCTGCGTGCGTCCACGTGCGTGTTTTCTGA
CTGACATGAAATCGACGCCCGAGTTAGCCTCACCCGGTGACCTCTAGCCCTGCCCGGATGGAGCGGGGCCCACCCGGT
TCAGTGTTTCTGGGGAGCTGGACAGTGGAGTGCAAAAGGCTTGCAGAACTTGAAGCCTGCTCCTTCCCTTGCTACCAC
GGCCTCCTTTCCGTTTGATTTGTCACTGCTTCAATCAATAACAGCCGCTCCAGAGTCAGTAGTCAATGAATATATGAC
CAAATATCACCAGGACTGTTACTCAATGTGTGCCGAGCCCTTGCCCATGCTGGGCTCCCGTGTATCTGGACACTGTAA
CGTGTGCTGTGTTTGCTCCCCTTCCCCTTCCTTCTTTGCCCTTTACTTGTCTTTCTGGGGTTTTTCTGTTTGGGTTTG
GTTTGGTTTTTATTTCTCCTTTTGTGTTCCAAACATGAGGTTCTCTCTACTGGTCCTCTTAACTGTGGTGTTGAGGCT
TATATTTGTGTAATTTTTGGTGGGTGAAAGGAATTTTGCTAAGTAAATCTCTTCTGTGTTTGAACTGAAGTCTGTATT
GTAACTATGTTTAAAGTAATTGTTCCAGAGACAAATATTTCTAGACACTTTTTCTTTACAAACAAAAGCATTCGGAGG
GAGGGGGATGGTGACTGAGATGAGAGGGGAGAGCTGAACAGATGACCCCTGCCCAGATCAGCCAGAAGCCACCCAAAG
CAGTGGAGCCCAGGAGTCCCACTCCAAGCCAGCAAGCCGAATAGCTGATGTGTTGCCACTTTCCAAGTCACTGCAAAA
CCAGGTTTTGTTCCGCCCAGTGGATTCTTGTTTTGCTTCCCCTCCCCCCGAGATTATTACCACCATCCCGTGCTTTTA
AGGAAAGGCAAGATTGATGTTTCCTTGAGGGGAGCCAGGAGGGGATGTGTGTGTGCAGAGCTGAAGAGCTGGGGAGAA
TGGGGCTGGGCCCACCCAAGCAGGAGGCTGGGACGCTCTGCTGTGGGCACAGGTCAGGCTAATGTTGGCAGATGCAGC
TCTTCCTGGACAGGCCAGGTGGTGGGCATTCTCTCTCCAAGGTGTGCCCCGTGGGCATTACTGTTTAAGACACTTCCG
TCACATCCCACCCCATCCTCCAGGGCTCAACACTGTGACATCTCTATTCCCCACCCTCCCCTTCCCAGGGCAATAAAA
TGACCATGGAGGGGGCTTGCACTCTCTTGGCTGTCACCCGATCGCCAGCAAAACTTAGATGTGAGAAAACCCCTTCCC
ATTCCATGGCGAAAACATCTCCTTAGAAAAGCCATTACCCTCATTAGGCATGGTTTTGGGCTCCCAAAACACCTGACA
GCCCCTCCCTCCTCTGAGAGGCGGAGAGTGCTGACTGTAGTGACCATTGCATGCCGGGTGCAGCATCTGGAAGAGCTA
GGCAGGGTGTCTGCCCCCTCCTGAGTTGAAGTCATGCTCCCCTGTGCCAGCCCAGAGGCCGAGAGCTATGGACAGCAT
TGCCAGTAACACAGGCCACCCTGTGCAGAAGGGAGCTGGCTCCAGCCTGGAAACCTGTCTGAGGTTGGGAGAGGTGCA
CTTGGGGCACAGGGAGAGGCCGGGACACACTTAGCTGGAGATGTCTCTAAAAGCCCTGTATCGTATTCACCTTCAGTT
TTTGTGTTTTGGGACAATTACTTTAGAAAATAAGTAGGTCGTTTTAAAAACAAAAATTATTGATTGCTTTTTTGTAGT
GTTCAGAAAAAAGGTTCTTTGTGTATAGCCAAATGACTGAAAGCACTGATATATTTAAAAACAAAAGGCAATTTATTA
AGGAAATTTGTACCATTTCAGTAAACCTGTCTGAATGTACCTGTATACGTTTCAAAAACACCCCCCCCCCACTGAATC
CCTGTAACCTATTTATTATATAAAGAGTTTGCCTTATAAATTTACATAAAAATGTCCGTTTGTGTCTTTTGTTGTAAA
AATCAAGTGATTTTTTCATAAGGTTCTTTTACTATTGGAAAAGATGGGCAGCACGCAGTTTTATTTTATTTTTGTAAG
TTTTTTAATACATGTGAAAGCAAAGAATACTCAGCATGCCTTTCTAAGTGACGCGTTTGCACCTTTTGTTGGGAAGTA
CTGTATCCTGTGCTGTTAGCATTCTCGATAAATCTCTCTGTGAAAGTGA
SEQ ID NO: 32 >NM_001369391.2 MECP2 [organism = Homo sapiens] [GeneID = 4204]
[transcript = 4]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGGACCCAGGAGGGAGAAGCAGCTGCCAGGGGAAGTCTCTTCGTAGGCGGAGGTCAGGAGTCCAAGAGGAGTGAGC
AGAGTCACAGAAGCCTCTTAAAGCCTCTTCTTCCCCCATCCCATCAACACATCTGCTGCCCAAGAACTCCTTTGTGAT
TGAATTTAACCAGAGTTGATTTCTGTTGCTTGAAACTCAGGACCTTGATGCAGTGTGCAGGGTCTGGGCAAGGAAATT
CATAAAAGAAGAGATACAGATGGCCAATGAAGATAGGAAAAGAGTTCTGCCTGCTGGTAATCAAAGAGATGCAAACGA
GAACAAAAATGATGCCTTTTCACCTACCAAATTTGTCAAGATTAAAAAGAAAGCAAAGAGCCAGCGTCAGCTGATGTT
CATACCTGCACCTGCTCGGTAGCTTGCTAATGTTCTGCCTGCTCCACACGCCAGGCCAGCCTCCACGGCGCAGCCAGG
GAAGAAAAGTCAGAAGACCAGGACCTCCAGGGCCTCAAGGACAAACCCCTCAAGTTTAAAAAGGTGAAGAAAGATAAG
AAAGAAGAGAAAGAGGGCAAGCATGAGCCCGTGCAGCCATCAGCCCACCACTCTGCTGAGCCCGCAGAGGCAGGCAAA
GCAGAGACATCAGAAGGGTCAGGCTCCGCCCCGGCTGTGCCGGAAGCTTCTGCCTCCCCCAAACAGCGGCGCTCCATC
ATCCGTGACCGGGGACCCATGTATGATGACCCCACCCTGCCTGAAGGCTGGACACGGAAGCTTAAGCAAAGGAAATCT
GGCCGCTCTGCTGGGAAGTATGATGTGTATTTGATCAATCCCCAGGGAAAAGCCTTTCGCTCTAAAGTGGAGTTGATT
GCGTACTTCGAAAAGGTAGGCGACACATCCCTGGACCCTAATGATTTTGACTTCACGGTAACTGGGAGAGGGAGCCCC
TCCCGGCGAGAGCAGAAACCACCTAAGAAGCCCAAATCTCCCAAAGCTCCAGGAACTGGCAGAGGCCGGGGACGCCCC
AAAGGGAGCGGCACCACGAGACCCAAGGCGGCCACGTCAGAGGGTGTGCAGGTGAAAAGGGTCCTGGAGAAAAGTCCT
GGGAAGCTCCTTGTCAAGATGCCTTTTCAAACTTCGCCAGGGGGCAAGGCTGAGGGGGGTGGGGCCACCACATCCACC
CAGGTCATGGTGATCAAACGCCCCGGCAGGAAGCGAAAAGCTGAGGCCGACCCTCAGGCCATTCCCAAGAAACGGGGC
CGAAAGCCGGGGAGTGTGGTGGCAGCCGCTGCCGCCGAGGCCAAAAAGAAAGCCGTGAAGGAGTCTTCTATCCGATCT
GTGCAGGAGACCGTACTCCCCATCAAGAAGCGCAAGACCCGGGAGACGGTCAGCATCGAGGTCAAGGAAGTGGTGAAG
CCCCTGCTGGTGTCCACCCTCGGTGAGAAGAGCGGGAAAGGACTGAAGACCTGTAAGAGCCCTGGGCGGAAAAGCAAG
GAGAGCAGCCCCAAGGGGCGCAGCAGCAGCGCCTCCTCACCCCCCAAGAAGGAGCACCACCACCATCACCACCACTCA
GAGTCCCCAAAGGCCCCCGTGCCACTGCTCCCACCCCTGCCCCCACCTCCACCTGAGCCCGAGAGCTCCGAGGACCCC
ACCAGCCCCCCTGAGCCCCAGGACTTGAGCAGCAGCGTCTGCAAAGAGGAGAAGATGCCCAGAGGAGGCTCACTGGAG
AGCGACGGCTGCCCCAAGGAGCCAGCTAAGACTCAGCCCGCGGTTGCCACCGCCGCCACGGCCGCAGAAAAGTACAAA
CACCGAGGGGAGGGAGAGCGCAAAGACATTGTTTCATCCTCCATGCCAAGGCCAAACAGAGAGGAGCCTGTGGACAGC
CGGACGCCCGTGACCGAGAGAGTTAGCTGACTTTACACGGAGCGGATTGCAAAGCAAACCAACAAGAATAAAGGCAGC
TGTTGTCTCTTCTCCTTATGGGTAGGGCTCTGACAAAGCTTCCCGATTAACTGAAATAAAAAATATTTTTTTTTCTTT
CAGTAAACTTAGAGTTTCGTGGCTTCAGGGTGGGAGTAGTTGGAGCATTGGGGATGTTTTTCTTACCGACAAGCACAG
TCAGGTTGAAGACCTAACCAGGGCCAGAAGTAGCTTTGCACTTTTCTAAACTAGGCTCCTTCAACAAGGCTTGCTGCA
GATACTACTGACCAGACAAGCTGTTGACCAGGCACCTCCCCTCCCGCCCAAACCTTTCCCCCATGTGGTCGTTAGAGA
CAGAGCGACAGAGCAGTTGAGAGGACACTCCCGTTTTCGGTGCCATCAGTGCCCCGTCTACAGCTCCCCCAGCTCCCC
CCACCTCCCCCACTCCCAACCACGTTGGGACAGGGAGGTGTGAGGCAGGAGAGACAGTTGGATTCTTTAGAGAAGATG
GATATGACCAGTGGCTATGGCCTGTGCGATCCCACCCGTGGTGGCTCAAGTCTGGCCCCACACCAGCCCCAATCCAAA
ACTGGCAAGGACGCTTCACAGGACAGGAAAGTGGCACCTGTCTGCTCCAGCTCTGGCATGGCTAGGAGGGGGGAGTCC
CTTGAACTACTGGGTGTAGACTGGCCTGAACCACAGGAGAGGATGGCCCAGGGTGAGGTGGCATGGTCCATTCTCAAG
GGACGTCCTCCAACGGGTGGCGCTAGAGGCCATGGAGGCAGTAGGACAAGGTGCAGGCAGGCTGGCCTGGGGTCAGGC
CGGGCAGAGCACAGCGGGGTGAGAGGGATTCCTAATCACTCAGAGCAGTCTGTGACTTAGTGGACAGGGGAGGGGGCA
AAGGGGGAGGAGAAGAAAATGTTCTTCCAGTTACTTTCCAATTCTCCTTTAGGGACAGCTTAGAATTATTTGCACTAT
TGAGTCTTCATGTTCCCACTTCAAAACAAACAGATGCTCTGAGAGCAAACTGGCTTGAATTGGTGACATTTAGTCCCT
CAAGCCACCAGATGTGACAGTGTTGAGAACTACCTGGATTTGTATATATACCTGCGCTTGTTTTAAAGTGGGCTCAGC
ACATAGGGTTCCCACGAAGCTCCGAAACTCTAAGTGTTTGCTGCAATTTTATAAGGACTTCCTGATTGGTTTCTCTTC
TCCCCTTCCATTTCTGCCTTTTGTTCATTTCATCCTTTCACTTCTTTCCCTTCCTCCATCCTCCTCCTTCCTAGTTCA
TCCCTTCTCTTCCAGGCAGCCGCGGTGCCCAACCACACTTGTCGGCTCCAGTCCCCAGAACTCTGCCTGCCCTTTGTC
CTCCTGCTGCCAGTACCAGCCCCACCCTGTTTTGAGCCCTGAGGAGGCCTTGGGCTCTGCTGAGTCCGACCTGGCCTG
TCTGTGAAGAGCAAGAGAGCAGCAAGGTCTTGCTCTCCTAGGTAGCCCCCTCTTCCCTGGTAAGAAAAAGCAAAAGGC
ATTTCCCACCCTGAACAACGAGCCTTTTCACCCTTCTACTCTAGAGAAGTGGACTGGAGGAGCTGGGCCCGATTTGGT
AGTTGAGGAAAGCACAGAGGCCTCCTGTGGCCTGCCAGTCATCGAGTGGCCCAACAGGGGCTCCATGCCAGCCGACCT
TGACCTCACTCAGAAGTCCAGAGTCTAGCGTAGTGCAGCAGGGCAGTAGCGGTACCAATGCAGAACTCCCAAGACCCG
AGCTGGGACCAGTACCTGGGTCCCCAGCCCTTCCTCTGCTCCCCCTTTTCCCTCGGAGTTCTTCTTGAATGGCAATGT
TTTGCTTTTGCTCGATGCAGACAGGGGGCCAGAACACCACACATTTCACTGTCTGTCTGGTCCATAGCTGTGGTGTAG
GGGCTTAGAGGCATGGGCTTGCTGTGGGTTTTTAATTGATCAGTTTTCATGTGGGATCCCATCTTTTTAACCTCTGTT
CAGGAAGTCCTTATCTAGCTGCATATCTTCATCATATTGGTATATCCTTTTCTGTGTTTACAGAGATGTCTCTTATAT
CTAAATCTGTCCAACTGAGAAGTACCTTATCAAAGTAGCAAATGAGACAGCAGTCTTATGCTTCCAGAAACACCCACA
GGCATGTCCCATGTGAGCTGCTGCCATGAACTGTCAAGTGTGTGTTGTCTTGTGTATTTCAGTTATTGTCCCTGGCTT
CCTTACTATGGTGTAATCATGAAGGAGTGAAACATCATAGAAACTGTCTAGCACTTCCTTGCCAGTCTTTAGTGATCA
GGAACCATAGTTGACAGTTCCAATCAGTAGCTTAAGAAAAAACCGTGTTTGTCTCTTCTGGAATGGTTAGAAGTGAGG
GAGTTTGCCCCGTTCTGTTTGTAGAGTCTCATAGTTGGACTTTCTAGCATATATGTGTCCATTTCCTTATGCTGTAAA
AGCAAGTCCTGCAACCAAACTCCCATCAGCCCAATCCCTGATCCCTGATCCCTTCCACCTGCTCTGCTGATGACCCCC
CCAGCTTCACTTCTGACTCTTCCCCAGGAAGGGAAGGGGGGTCAGAAGAGAGGGTGAGTCCTCCAGAACTCTTCCTCC
AAGGACAGAAGGCTCCTGCCCCCATAGTGGCCTCGAACTCCTGGCACTACCAAAGGACACTTATCCACGAGAGCGCAG
CATCCGACCAGGTTGTCACTGAGAAGATGTTTATTTTGGTCAGTTGGGTTTTTATGTATTATACTTAGTCAAATGTAA
TGTGGCTTCTGGAATCATTGTCCAGAGCTGCTTCCCCGTCACCTGGGCGTCATCTGGTCCTGGTAAGAGGAGTGCGTG
GCCCACCAGGCCCCCCTGTCACCCATGACAGTTCATTCAGGGCCGATGGGGCAGTCGTGGTTGGGAACACAGCATTTC
AAGCGTCACTTTATTTCATTCGGGCCCCACCTGCAGCTCCCTCAAAGAGGCAGTTGCCCAGCCTCTTTCCCTTCCAGT
TTATTCCAGAGCTGCCAGTGGGGCCTGAGGCTCCTTAGGGTTTTCTCTCTATTTCCCCCTTTCTTCCTCATTCCCTCG
TCTTTCCCAAAGGCATCACGAGTCAGTCGCCTTTCAGCAGGCAGCCTTGGCGGTTTATCGCCCTGGCAGGCAGGGGCC
CTGCAGCTCTCATGCTGCCCCTGCCTTGGGGTCAGGTTGACAGGAGGTTGGAGGGAAAGCCTTAAGCTGCAGGATTCT
CACCAGCTGTGTCCGGCCCAGTTTTGGGGTGTGACCTCAATTTCAATTTTGTCTGTACTTGAACATTATGAAGATGGG
GGCCTCTTTCAGTGAATTTGTGAACAGCAGAATTGACCGACAGCTTTCCAGTACCCATGGGGCTAGGTCATTAAGGCC
ACATCCACAGTCTCCCCCACCCTTGTTCCAGTTGTTAGTTACTACCTCCTCTCCTGACAATACTGTATGTCGTCGAGC
TCCCCCCAGGTCTACCCCTCCCGGCCCTGCCTGCTGGTGGGCTTGTCATAGCCAGTGGGATTGCCGGTCTTGACAGCT
CAGTGAGCTGGAGATACTTGGTCACAGCCAGGCGCTAGCACAGCTCCCTTCTGTTGATGCTGTATTCCCATATCAAAA
GACACAGGGGACACCCAGAAACGCCACATCCCCCAATCCATCAGTGCCAAACTAGCCAACGGCCCCAGCTTCTCAGCT
CGCTGGATGGCGGAAGCTGCTACTCGTGAGCGCCAGTGCGGGTGCAGACAATCTTCTGTTGGGTGGCATCATTCCAGG
CCCGAAGCATGAACAGTGCACCTGGGACAGGGAGCAGCCCCAAATTGTCACCTGCTTCTCTGCCCAGCTTTTCATTGC
TGTGACAGTGATGGCGAAAGAGGGTAATAACCAGACACAAACTGCCAAGTTGGGTGGAGAAAGGAGTTTCTTTAGCTG
ACAGAATCTCTGAATTTTAAATCACTTAGTAAGCGGCTCAAGCCCAGGAGGGAGCAGAGGGATACGAGCGGAGTCCCC
TGCGCGGGACCATCTGGAATTGGTTTAGCCCAAGTGGAGCCTGACAGCCAGAACTCTGTGTCCCCCGTCTAACCACAG
CTCCTTTTCCAGAGCATTCCAGTCAGGCTCTCTGGGCTGACTGGGCCAGGGGAGGTTACAGGTACCAGTTCTTTAAGA
AGATCTTTGGGCATATACATTTTTAGCCTGTGTCATTGCCCCAAATGGATTCCTGTTTCAAGTTCACACCTGCAGATT
CTAGGACCTGTGTCCTAGACTTCAGGGAGTCAGCTGTTTCTAGAGTTCCTACCATGGAGTGGGTCTGGAGGACCTGCC
CGGTGGGGGGGCAGAGCCCTGCTCCCTCCGGGTCTTCCTACTCTTCTCTCTGCTCTGACGGGATTTGTTGATTCTCTC
CATTTTGGTGTCTTTCTCTTTTAGATATTGTATCAATCTTTAGAAAAGGCATAGTCTACTTGTTATAAATCGTTAGGA
TACTGCCTCCCCCAGGGTCTAAAATTACATATTAGAGGGGAAAAGCTGAACACTGAAGTCAGTTCTCAACAATTTAGA
AGGAAAACCTAGAAAACATTTGGCAGAAAATTACATTTCGATGTTTTTGAATGAATACGAGCAAGCTTTTACAACAGT
GCTGATCTAAAAATACTTAGCACTTGGCCTGAGATGCCTGGTGAGCATTACAGGCAAGGGGAATCTGGAGGTAGCCGA
CCTGAGGACATGGCTTCTGAACCTGTCTTTTGGGAGTGGTATGGAAGGTGGAGCGTTCACCAGTGACCTGGAAGGCCC
AGCACCACCCTCCTTCCCACTCTTCTCATCTTGACAGAGCCTGCCCCAGCGCTGACGTGTCAGGAAAACACCCAGGGA
ACTAGGAAGGCACTTCTGCCTGAGGGGCAGCCTGCCTTGCCCACTCCTGCTCTGCTCGCCTCGGATCAGCTGAGCCTT
CTGAGCTGGCCTCTCACTGCCTCCCCAAGGCCCCCTGCCTGCCCTGTCAGGAGGCAGAAGGAAGCAGGTGTGAGGGCA
GTGCAAGGAGGGAGCACAACCCCCAGCTCCCGCTCCGGGCTCCGACTTGTGCACAGGCAGAGCCCAGACCCTGGAGGA
AATCCTACCTTTGAATTCAAGAACATTTGGGGAATTTGGAAATCTCTTTGCCCCCAAACCCCCATTCTGTCCTACCTT
TAATCAGGTCCTGCTCAGCAGTGAGAGCAGATGAGGTGAAAAGGCCAAGAGGTTTGGCTCCTGCCCACTGATAGCCCC
TCTCCCCGCAGTGTTTGTGTGTCAAGTGGCAAAGCTGTTCTTCCTGGTGACCCTGATTATATCCAGTAACACATAGAC
TGTGCGCATAGGCCTGCTTTGTCTCCTCTATCCTGGGCTTTTGTTTTGCTTTTTAGTTTTGCTTTTAGTTTTTCTGTC
CCTTTTATTTAACGCACCGACTAGACACACAAAGCAGTTGAATTTTTATATATATATCTGTATATTGCACAATTATAA
ACTCATTTTGCTTGTGGCTCCACACACACAAAAAAAGACCTGTTAAAATTATACCTGTTGCTTAATTACAATATTTCT
GATAACCATAGCATAGGACAAGGGAAAATAAAAAAAGAAAAAAAAGAAAAAAAAACGACAAATCTGTCTGCTGGTCAC
TTCTTCTGTCCAAGCAGATTCGTGGTCTTTTCCTCGCTTCTTTCAAGGGCTTTCCTGTGCCAGGTGAAGGAGGCTCCA
GGCAGCACCCAGGTTTTGCACTCTTGTTTCTCCCGTGCTTGTGAAAGAGGTCCCAAGGTTCTGGGTGCAGGAGCGCTC
CCTTGACCTGCTGAAGTCCGGAACGTAGTCGGCACAGCCTGGTCGCCTTCCACCTCTGGGAGCTGGAGTCCACTGGGG
TGGCCTGACTCCCCCAGTCCCCTTCCCGTGACCTGGTCAGGGTGAGCCCATGTGGAGTCAGCCTCGCAGGCCTCCCTG
CCAGTAGGGTCCGAGTGTGTTTCATCCTTCCCACTCTGTCGAGCCTGGGGGCTGGAGCGGAGACGGGAGGCCTGGCCT
GTCTCGGAACCTGTGAGCTGCACCAGGTAGAACGCCAGGGACCCCAGAATCATGTGCGTCAGTCCAAGGGGTCCCCTC
CAGGAGTAGTGAAGACTCCAGAAATGTCCCTTTCTTCTCCCCCATCCTACGAGTAATTGCATTTGCTTTTGTAATTCT
TAATGAGCAATATCTGCTAGAGAGTTTAGCTGTAACAGTTCTTTTTGATCATCTTTTTTTAATAATTAGAAACACCAA
AAAAATCCAGAAACTTGTTCTTCCAAAGCAGAGAGCATTATAATCACCAGGGCCAAAAGCTTCCCTCCCTGCTGTCAT
TGCTTCTTCTGAGGCCTGAATCCAAAAGAAAAACAGCCATAGGCCCTTTCAGTGGCCGGGCTACCCGTGAGCCCTTCG
GAGGACCAGGGCTGGGGCAGCCTCTGGGCCCACATCCGGGGCCAGCTCCGGCGTGTGTTCAGTGTTAGCAGTGGGTCA
TGATGCTCTTTCCCACCCAGCCTGGGATAGGGGCAGAGGAGGCGAGGAGGCCGTTGCCGCTGATGTTTGGCCGTGAAC
AGGTGGGTGTCTGCGTGCGTCCACGTGCGTGTTTTCTGACTGACATGAAATCGACGCCCGAGTTAGCCTCACCCGGTG
ACCTCTAGCCCTGCCCGGATGGAGCGGGGCCCACCCGGTTCAGTGTTTCTGGGGAGCTGGACAGTGGAGTGCAAAAGG
CTTGCAGAACTTGAAGCCTGCTCCTTCCCTTGCTACCACGGCCTCCTTTCCGTTTGATTTGTCACTGCTTCAATCAAT
AACAGCCGCTCCAGAGTCAGTAGTCAATGAATATATGACCAAATATCACCAGGACTGTTACTCAATGTGTGCCGAGCC
CTTGCCCATGCTGGGCTCCCGTGTATCTGGACACTGTAACGTGTGCTGTGTTTGCTCCCCTTCCCCTTCCTTCTTTGC
CCTTTACTTGTCTTTCTGGGGTTTTTCTGTTTGGGTTTGGTTTGGTTTTTATTTCTCCTTTTGTGTTCCAAACATGAG
GTTCTCTCTACTGGTCCTCTTAACTGTGGTGTTGAGGCTTATATTTGTGTAATTTTTGGTGGGTGAAAGGAATTTTGC
TAAGTAAATCTCTTCTGTGTTTGAACTGAAGTCTGTATTGTAACTATGTTTAAAGTAATTGTTCCAGAGACAAATATT
TCTAGACACTTTTTCTTTACAAACAAAAGCATTCGGAGGGAGGGGGATGGTGACTGAGATGAGAGGGGAGAGCTGAAC
AGATGACCCCTGCCCAGATCAGCCAGAAGCCACCCAAAGCAGTGGAGCCCAGGAGTCCCACTCCAAGCCAGCAAGCCG
AATAGCTGATGTGTTGCCACTTTCCAAGTCACTGCAAAACCAGGTTTTGTTCCGCCCAGTGGATTCTTGTTTTGCTTC
CCCTCCCCCCGAGATTATTACCACCATCCCGTGCTTTTAAGGAAAGGCAAGATTGATGTTTCCTTGAGGGGAGCCAGG
AGGGGATGTGTGTGTGCAGAGCTGAAGAGCTGGGGAGAATGGGGCTGGGCCCACCCAAGCAGGAGGCTGGGACGCTCT
GCTGTGGGCACAGGTCAGGCTAATGTTGGCAGATGCAGCTCTTCCTGGACAGGCCAGGTGGTGGGCATTCTCTCTCCA
AGGTGTGCCCCGTGGGCATTACTGTTTAAGACACTTCCGTCACATCCCACCCCATCCTCCAGGGCTCAACACTGTGAC
ATCTCTATTCCCCACCCTCCCCTTCCCAGGGCAATAAAATGACCATGGAGGGGGCTTGCACTCTCTTGGCTGTCACCC
GATCGCCAGCAAAACTTAGATGTGAGAAAACCCCTTCCCATTCCATGGCGAAAACATCTCCTTAGAAAAGCCATTACC
CTCATTAGGCATGGTTTTGGGCTCCCAAAACACCTGACAGCCCCTCCCTCCTCTGAGAGGCGGAGAGTGCTGACTGTA
GTGACCATTGCATGCCGGGTGCAGCATCTGGAAGAGCTAGGCAGGGTGTCTGCCCCCTCCTGAGTTGAAGTCATGCTC
CCCTGTGCCAGCCCAGAGGCCGAGAGCTATGGACAGCATTGCCAGTAACACAGGCCACCCTGTGCAGAAGGGAGCTGG
CTCCAGCCTGGAAACCTGTCTGAGGTTGGGAGAGGTGCACTTGGGGCACAGGGAGAGGCCGGGACACACTTAGCTGGA
GATGTCTCTAAAAGCCCTGTATCGTATTCACCTTCAGTTTTTGTGTTTTGGGACAATTACTTTAGAAAATAAGTAGGT
CGTTTTAAAAACAAAAATTATTGATTGCTTTTTTGTAGTGTTCAGAAAAAAGGTTCTTTGTGTATAGCCAAATGACTG
AAAGCACTGATATATTTAAAAACAAAAGGCAATTTATTAAGGAAATTTGTACCATTTCAGTAAACCTGTCTGAATGTA
CCTGTATACGTTTCAAAAACACCCCCCCCCCACTGAATCCCTGTAACCTATTTATTATATAAAGAGTTTGCCTTATAA
ATTTACATAAAAATGTCCGTTTGTGTCTTTTGTTGTAAAAATCAAGTGATTTTTTCATAAGGTTCTTTTACTATTGGA
AAAGATGGGCAGCACGCAGTTTTATTTTATTTTTGTAAGTTTTTTAATACATGTGAAAGCAAAGAATACTCAGCATGC
CTTTCTAAGTGACGCGTTTGCACCTTTTGTTGGGAAGTACTGTATCCTGTGCTGTTAGCATTCTCGATAAATCTCTCT
GTGAAAGTGA
SEQ ID NO: 33 >NM_001316337.2 MECP2 [organism = Homo sapiens] [GeneID = 4204]
[transcript = 3]
GCGCGCGCTCCCTCCTCTCGGAGAGAGGGCTGTGGTAAAAGCCGTCCGGAAAATGGCCGCCGCCGCCGCCGCCGCGCC
GAGCGGAGGAGGAGGAGGAGGCGAGGAGGAGAGACTGCTCCATAAAAATACAGACTCACCAGTTCCTGCTTTGATGTG
ACATGTGACTCCCCAGAATACACCTTGCTTCTGTAGACCAGCTCCAACAGGATTCCATGGTAGCTGGGATGTTAGGGC
TCAGCTGCAAGATGGGATTCAGATCTGTTCTCAAGCCTGTCGTTCCAGGACCCAGGAGGGAGAAGCAGCTGCCAGGGG
AAGTCTCTTCGTAGGCGGAGGTCAGGAGTCCAAGAGGAGTGAGCAGAGTCACAGAAGCCTCTTAAAGCCTCTTCTTCC
CCCATCCCATCAACACGGAAGAAAAGTCAGAAGACCAGGACCTCCAGGGCCTCAAGGACAAACCCCTCAAGTTTAAAA
AGGTGAAGAAAGATAAGAAAGAAGAGAAAGAGGGCAAGCATGAGCCCGTGCAGCCATCAGCCCACCACTCTGCTGAGC
CCGCAGAGGCAGGCAAAGCAGAGACATCAGAAGGGTCAGGCTCCGCCCCGGCTGTGCCGGAAGCTTCTGCCTCCCCCA
AACAGCGGCGCTCCATCATCCGTGACCGGGGACCCATGTATGATGACCCCACCCTGCCTGAAGGCTGGACACGGAAGC
TTAAGCAAAGGAAATCTGGCCGCTCTGCTGGGAAGTATGATGTGTATTTGATCAATCCCCAGGGAAAAGCCTTTCGCT
CTAAAGTGGAGTTGATTGCGTACTTCGAAAAGGTAGGCGACACATCCCTGGACCCTAATGATTTTGACTTCACGGTAA
CTGGGAGAGGGAGCCCCTCCCGGCGAGAGCAGAAACCACCTAAGAAGCCCAAATCTCCCAAAGCTCCAGGAACTGGCA
GAGGCCGGGGACGCCCCAAAGGGAGCGGCACCACGAGACCCAAGGCGGCCACGTCAGAGGGTGTGCAGGTGAAAAGGG
TCCTGGAGAAAAGTCCTGGGAAGCTCCTTGTCAAGATGCCTTTTCAAACTTCGCCAGGGGGCAAGGCTGAGGGGGGTG
GGGCCACCACATCCACCCAGGTCATGGTGATCAAACGCCCCGGCAGGAAGCGAAAAGCTGAGGCCGACCCTCAGGCCA
TTCCCAAGAAACGGGGCCGAAAGCCGGGGAGTGTGGTGGCAGCCGCTGCCGCCGAGGCCAAAAAGAAAGCCGTGAAGG
AGTCTTCTATCCGATCTGTGCAGGAGACCGTACTCCCCATCAAGAAGCGCAAGACCCGGGAGACGGTCAGCATCGAGG
TCAAGGAAGTGGTGAAGCCCCTGCTGGTGTCCACCCTCGGTGAGAAGAGCGGGAAAGGACTGAAGACCTGTAAGAGCC
CTGGGCGGAAAAGCAAGGAGAGCAGCCCCAAGGGGCGCAGCAGCAGCGCCTCCTCACCCCCCAAGAAGGAGCACCACC
ACCATCACCACCACTCAGAGTCCCCAAAGGCCCCCGTGCCACTGCTCCCACCCCTGCCCCCACCTCCACCTGAGCCCG
AGAGCTCCGAGGACCCCACCAGCCCCCCTGAGCCCCAGGACTTGAGCAGCAGCGTCTGCAAAGAGGAGAAGATGCCCA
GAGGAGGCTCACTGGAGAGCGACGGCTGCCCCAAGGAGCCAGCTAAGACTCAGCCCGCGGTTGCCACCGCCGCCACGG
CCGCAGAAAAGTACAAACACCGAGGGGAGGGAGAGCGCAAAGACATTGTTTCATCCTCCATGCCAAGGCCAAACAGAG
AGGAGCCTGTGGACAGCCGGACGCCCGTGACCGAGAGAGTTAGCTGACTTTACACGGAGCGGATTGCAAAGCAAACCA
ACAAGAATAAAGGCAGCTGTTGTCTCTTCTCCTTATGGGTAGGGCTCTGACAAAGCTTCCCGATTAACTGAAATAAAA
AATATTTTTTTTTCTTTCAGTAAACTTAGAGTTTCGTGGCTTCAGGGTGGGAGTAGTTGGAGCATTGGGGATGTTTTT
CTTACCGACAAGCACAGTCAGGTTGAAGACCTAACCAGGGCCAGAAGTAGCTTTGCACTTTTCTAAACTAGGCTCCTT
CAACAAGGCTTGCTGCAGATACTACTGACCAGACAAGCTGTTGACCAGGCACCTCCCCTCCCGCCCAAACCTTTCCCC
CATGTGGTCGTTAGAGACAGAGCGACAGAGCAGTTGAGAGGACACTCCCGTTTTCGGTGCCATCAGTGCCCCGTCTAC
AGCTCCCCCAGCTCCCCCCACCTCCCCCACTCCCAACCACGTTGGGACAGGGAGGTGTGAGGCAGGAGAGACAGTTGG
ATTCTTTAGAGAAGATGGATATGACCAGTGGCTATGGCCTGTGCGATCCCACCCGTGGTGGCTCAAGTCTGGCCCCAC
ACCAGCCCCAATCCAAAACTGGCAAGGACGCTTCACAGGACAGGAAAGTGGCACCTGTCTGCTCCAGCTCTGGCATGG
CTAGGAGGGGGGAGTCCCTTGAACTACTGGGTGTAGACTGGCCTGAACCACAGGAGAGGATGGCCCAGGGTGAGGTGG
CATGGTCCATTCTCAAGGGACGTCCTCCAACGGGTGGCGCTAGAGGCCATGGAGGCAGTAGGACAAGGTGCAGGCAGG
CTGGCCTGGGGTCAGGCCGGGCAGAGCACAGCGGGGTGAGAGGGATTCCTAATCACTCAGAGCAGTCTGTGACTTAGT
GGACAGGGGAGGGGGCAAAGGGGGAGGAGAAGAAAATGTTCTTCCAGTTACTTTCCAATTCTCCTTTAGGGACAGCTT
AGAATTATTTGCACTATTGAGTCTTCATGTTCCCACTTCAAAACAAACAGATGCTCTGAGAGCAAACTGGCTTGAATT
GGTGACATTTAGTCCCTCAAGCCACCAGATGTGACAGTGTTGAGAACTACCTGGATTTGTATATATACCTGCGCTTGT
TTTAAAGTGGGCTCAGCACATAGGGTTCCCACGAAGCTCCGAAACTCTAAGTGTTTGCTGCAATTTTATAAGGACTTC
CTGATTGGTTTCTCTTCTCCCCTTCCATTTCTGCCTTTTGTTCATTTCATCCTTTCACTTCTTTCCCTTCCTCCATCC
TCCTCCTTCCTAGTTCATCCCTTCTCTTCCAGGCAGCCGCGGTGCCCAACCACACTTGTCGGCTCCAGTCCCCAGAAC
TCTGCCTGCCCTTTGTCCTCCTGCTGCCAGTACCAGCCCCACCCTGTTTTGAGCCCTGAGGAGGCCTTGGGCTCTGCT
GAGTCCGACCTGGCCTGTCTGTGAAGAGCAAGAGAGCAGCAAGGTCTTGCTCTCCTAGGTAGCCCCCTCTTCCCTGGT
AAGAAAAAGCAAAAGGCATTTCCCACCCTGAACAACGAGCCTTTTCACCCTTCTACTCTAGAGAAGTGGACTGGAGGA
GCTGGGCCCGATTTGGTAGTTGAGGAAAGCACAGAGGCCTCCTGTGGCCTGCCAGTCATCGAGTGGCCCAACAGGGGC
TCCATGCCAGCCGACCTTGACCTCACTCAGAAGTCCAGAGTCTAGCGTAGTGCAGCAGGGCAGTAGCGGTACCAATGC
AGAACTCCCAAGACCCGAGCTGGGACCAGTACCTGGGTCCCCAGCCCTTCCTCTGCTCCCCCTTTTCCCTCGGAGTTC
TTCTTGAATGGCAATGTTTTGCTTTTGCTCGATGCAGACAGGGGGCCAGAACACCACACATTTCACTGTCTGTCTGGT
CCATAGCTGTGGTGTAGGGGCTTAGAGGCATGGGCTTGCTGTGGGTTTTTAATTGATCAGTTTTCATGTGGGATCCCA
TCTTTTTAACCTCTGTTCAGGAAGTCCTTATCTAGCTGCATATCTTCATCATATTGGTATATCCTTTTCTGTGTTTAC
AGAGATGTCTCTTATATCTAAATCTGTCCAACTGAGAAGTACCTTATCAAAGTAGCAAATGAGACAGCAGTCTTATGC
TTCCAGAAACACCCACAGGCATGTCCCATGTGAGCTGCTGCCATGAACTGTCAAGTGTGTGTTGTCTTGTGTATTTCA
GTTATTGTCCCTGGCTTCCTTACTATGGTGTAATCATGAAGGAGTGAAACATCATAGAAACTGTCTAGCACTTCCTTG
CCAGTCTTTAGTGATCAGGAACCATAGTTGACAGTTCCAATCAGTAGCTTAAGAAAAAACCGTGTTTGTCTCTTCTGG
AATGGTTAGAAGTGAGGGAGTTTGCCCCGTTCTGTTTGTAGAGTCTCATAGTTGGACTTTCTAGCATATATGTGTCCA
TTTCCTTATGCTGTAAAAGCAAGTCCTGCAACCAAACTCCCATCAGCCCAATCCCTGATCCCTGATCCCTTCCACCTG
CTCTGCTGATGACCCCCCCAGCTTCACTTCTGACTCTTCCCCAGGAAGGGAAGGGGGGTCAGAAGAGAGGGTGAGTCC
TCCAGAACTCTTCCTCCAAGGACAGAAGGCTCCTGCCCCCATAGTGGCCTCGAACTCCTGGCACTACCAAAGGACACT
TATCCACGAGAGCGCAGCATCCGACCAGGTTGTCACTGAGAAGATGTTTATTTTGGTCAGTTGGGTTTTTATGTATTA
TACTTAGTCAAATGTAATGTGGCTTCTGGAATCATTGTCCAGAGCTGCTTCCCCGTCACCTGGGCGTCATCTGGTCCT
GGTAAGAGGAGTGCGTGGCCCACCAGGCCCCCCTGTCACCCATGACAGTTCATTCAGGGCCGATGGGGCAGTCGTGGT
TGGGAACACAGCATTTCAAGCGTCACTTTATTTCATTCGGGCCCCACCTGCAGCTCCCTCAAAGAGGCAGTTGCCCAG
CCTCTTTCCCTTCCAGTTTATTCCAGAGCTGCCAGTGGGGCCTGAGGCTCCTTAGGGTTTTCTCTCTATTTCCCCCTT
TCTTCCTCATTCCCTCGTCTTTCCCAAAGGCATCACGAGTCAGTCGCCTTTCAGCAGGCAGCCTTGGCGGTTTATCGC
CCTGGCAGGCAGGGGCCCTGCAGCTCTCATGCTGCCCCTGCCTTGGGGTCAGGTTGACAGGAGGTTGGAGGGAAAGCC
TTAAGCTGCAGGATTCTCACCAGCTGTGTCCGGCCCAGTTTTGGGGTGTGACCTCAATTTCAATTTTGTCTGTACTTG
AACATTATGAAGATGGGGGCCTCTTTCAGTGAATTTGTGAACAGCAGAATTGACCGACAGCTTTCCAGTACCCATGGG
GCTAGGTCATTAAGGCCACATCCACAGTCTCCCCCACCCTTGTTCCAGTTGTTAGTTACTACCTCCTCTCCTGACAAT
ACTGTATGTCGTCGAGCTCCCCCCAGGTCTACCCCTCCCGGCCCTGCCTGCTGGTGGGCTTGTCATAGCCAGTGGGAT
TGCCGGTCTTGACAGCTCAGTGAGCTGGAGATACTTGGTCACAGCCAGGCGCTAGCACAGCTCCCTTCTGTTGATGCT
GTATTCCCATATCAAAAGACACAGGGGACACCCAGAAACGCCACATCCCCCAATCCATCAGTGCCAAACTAGCCAACG
GCCCCAGCTTCTCAGCTCGCTGGATGGCGGAAGCTGCTACTCGTGAGCGCCAGTGCGGGTGCAGACAATCTTCTGTTG
GGTGGCATCATTCCAGGCCCGAAGCATGAACAGTGCACCTGGGACAGGGAGCAGCCCCAAATTGTCACCTGCTTCTCT
GCCCAGCTTTTCATTGCTGTGACAGTGATGGCGAAAGAGGGTAATAACCAGACACAAACTGCCAAGTTGGGTGGAGAA
AGGAGTTTCTTTAGCTGACAGAATCTCTGAATTTTAAATCACTTAGTAAGCGGCTCAAGCCCAGGAGGGAGCAGAGGG
ATACGAGCGGAGTCCCCTGCGCGGGACCATCTGGAATTGGTTTAGCCCAAGTGGAGCCTGACAGCCAGAACTCTGTGT
CCCCCGTCTAACCACAGCTCCTTTTCCAGAGCATTCCAGTCAGGCTCTCTGGGCTGACTGGGCCAGGGGAGGTTACAG
GTACCAGTTCTTTAAGAAGATCTTTGGGCATATACATTTTTAGCCTGTGTCATTGCCCCAAATGGATTCCTGTTTCAA
GTTCACACCTGCAGATTCTAGGACCTGTGTCCTAGACTTCAGGGAGTCAGCTGTTTCTAGAGTTCCTACCATGGAGTG
GGTCTGGAGGACCTGCCCGGTGGGGGGGCAGAGCCCTGCTCCCTCCGGGTCTTCCTACTCTTCTCTCTGCTCTGACGG
GATTTGTTGATTCTCTCCATTTTGGTGTCTTTCTCTTTTAGATATTGTATCAATCTTTAGAAAAGGCATAGTCTACTT
GTTATAAATCGTTAGGATACTGCCTCCCCCAGGGTCTAAAATTACATATTAGAGGGGAAAAGCTGAACACTGAAGTCA
GTTCTCAACAATTTAGAAGGAAAACCTAGAAAACATTTGGCAGAAAATTACATTTCGATGTTTTTGAATGAATACGAG
CAAGCTTTTACAACAGTGCTGATCTAAAAATACTTAGCACTTGGCCTGAGATGCCTGGTGAGCATTACAGGCAAGGGG
AATCTGGAGGTAGCCGACCTGAGGACATGGCTTCTGAACCTGTCTTTTGGGAGTGGTATGGAAGGTGGAGCGTTCACC
AGTGACCTGGAAGGCCCAGCACCACCCTCCTTCCCACTCTTCTCATCTTGACAGAGCCTGCCCCAGCGCTGACGTGTC
AGGAAAACACCCAGGGAACTAGGAAGGCACTTCTGCCTGAGGGGCAGCCTGCCTTGCCCACTCCTGCTCTGCTCGCCT
CGGATCAGCTGAGCCTTCTGAGCTGGCCTCTCACTGCCTCCCCAAGGCCCCCTGCCTGCCCTGTCAGGAGGCAGAAGG
AAGCAGGTGTGAGGGCAGTGCAAGGAGGGAGCACAACCCCCAGCTCCCGCTCCGGGCTCCGACTTGTGCACAGGCAGA
GCCCAGACCCTGGAGGAAATCCTACCTTTGAATTCAAGAACATTTGGGGAATTTGGAAATCTCTTTGCCCCCAAACCC
CCATTCTGTCCTACCTTTAATCAGGTCCTGCTCAGCAGTGAGAGCAGATGAGGTGAAAAGGCCAAGAGGTTTGGCTCC
TGCCCACTGATAGCCCCTCTCCCCGCAGTGTTTGTGTGTCAAGTGGCAAAGCTGTTCTTCCTGGTGACCCTGATTATA
TCCAGTAACACATAGACTGTGCGCATAGGCCTGCTTTGTCTCCTCTATCCTGGGCTTTTGTTTTGCTTTTTAGTTTTG
CTTTTAGTTTTTCTGTCCCTTTTATTTAACGCACCGACTAGACACACAAAGCAGTTGAATTTTTATATATATATCTGT
ATATTGCACAATTATAAACTCATTTTGCTTGTGGCTCCACACACACAAAAAAAGACCTGTTAAAATTATACCTGTTGC
TTAATTACAATATTTCTGATAACCATAGCATAGGACAAGGGAAAATAAAAAAAGAAAAAAAAGAAAAAAAAACGACAA
ATCTGTCTGCTGGTCACTTCTTCTGTCCAAGCAGATTCGTGGTCTTTTCCTCGCTTCTTTCAAGGGCTTTCCTGTGCC
AGGTGAAGGAGGCTCCAGGCAGCACCCAGGTTTTGCACTCTTGTTTCTCCCGTGCTTGTGAAAGAGGTCCCAAGGTTC
TGGGTGCAGGAGCGCTCCCTTGACCTGCTGAAGTCCGGAACGTAGTCGGCACAGCCTGGTCGCCTTCCACCTCTGGGA
GCTGGAGTCCACTGGGGTGGCCTGACTCCCCCAGTCCCCTTCCCGTGACCTGGTCAGGGTGAGCCCATGTGGAGTCAG
CCTCGCAGGCCTCCCTGCCAGTAGGGTCCGAGTGTGTTTCATCCTTCCCACTCTGTCGAGCCTGGGGGCTGGAGCGGA
GACGGGAGGCCTGGCCTGTCTCGGAACCTGTGAGCTGCACCAGGTAGAACGCCAGGGACCCCAGAATCATGTGCGTCA
GTCCAAGGGGTCCCCTCCAGGAGTAGTGAAGACTCCAGAAATGTCCCTTTCTTCTCCCCCATCCTACGAGTAATTGCA
TTTGCTTTTGTAATTCTTAATGAGCAATATCTGCTAGAGAGTTTAGCTGTAACAGTTCTTTTTGATCATCTTTTTTTA
ATAATTAGAAACACCAAAAAAATCCAGAAACTTGTTCTTCCAAAGCAGAGAGCATTATAATCACCAGGGCCAAAAGCT
TCCCTCCCTGCTGTCATTGCTTCTTCTGAGGCCTGAATCCAAAAGAAAAACAGCCATAGGCCCTTTCAGTGGCCGGGC
TACCCGTGAGCCCTTCGGAGGACCAGGGCTGGGGCAGCCTCTGGGCCCACATCCGGGGCCAGCTCCGGCGTGTGTTCA
GTGTTAGCAGTGGGTCATGATGCTCTTTCCCACCCAGCCTGGGATAGGGGCAGAGGAGGCGAGGAGGCCGTTGCCGCT
GATGTTTGGCCGTGAACAGGTGGGTGTCTGCGTGCGTCCACGTGCGTGTTTTCTGACTGACATGAAATCGACGCCCGA
GTTAGCCTCACCCGGTGACCTCTAGCCCTGCCCGGATGGAGCGGGGCCCACCCGGTTCAGTGTTTCTGGGGAGCTGGA
CAGTGGAGTGCAAAAGGCTTGCAGAACTTGAAGCCTGCTCCTTCCCTTGCTACCACGGCCTCCTTTCCGTTTGATTTG
TCACTGCTTCAATCAATAACAGCCGCTCCAGAGTCAGTAGTCAATGAATATATGACCAAATATCACCAGGACTGTTAC
TCAATGTGTGCCGAGCCCTTGCCCATGCTGGGCTCCCGTGTATCTGGACACTGTAACGTGTGCTGTGTTTGCTCCCCT
TCCCCTTCCTTCTTTGCCCTTTACTTGTCTTTCTGGGGTTTTTCTGTTTGGGTTTGGTTTGGTTTTTATTTCTCCTTT
TGTGTTCCAAACATGAGGTTCTCTCTACTGGTCCTCTTAACTGTGGTGTTGAGGCTTATATTTGTGTAATTTTTGGTG
GGTGAAAGGAATTTTGCTAAGTAAATCTCTTCTGTGTTTGAACTGAAGTCTGTATTGTAACTATGTTTAAAGTAATTG
TTCCAGAGACAAATATTTCTAGACACTTTTTCTTTACAAACAAAAGCATTCGGAGGGAGGGGGATGGTGACTGAGATG
AGAGGGGAGAGCTGAACAGATGACCCCTGCCCAGATCAGCCAGAAGCCACCCAAAGCAGTGGAGCCCAGGAGTCCCAC
TCCAAGCCAGCAAGCCGAATAGCTGATGTGTTGCCACTTTCCAAGTCACTGCAAAACCAGGTTTTGTTCCGCCCAGTG
GATTCTTGTTTTGCTTCCCCTCCCCCCGAGATTATTACCACCATCCCGTGCTTTTAAGGAAAGGCAAGATTGATGTTT
CCTTGAGGGGAGCCAGGAGGGGATGTGTGTGTGCAGAGCTGAAGAGCTGGGGAGAATGGGGCTGGGCCCACCCAAGCA
GGAGGCTGGGACGCTCTGCTGTGGGCACAGGTCAGGCTAATGTTGGCAGATGCAGCTCTTCCTGGACAGGCCAGGTGG
TGGGCATTCTCTCTCCAAGGTGTGCCCCGTGGGCATTACTGTTTAAGACACTTCCGTCACATCCCACCCCATCCTCCA
GGGCTCAACACTGTGACATCTCTATTCCCCACCCTCCCCTTCCCAGGGCAATAAAATGACCATGGAGGGGGCTTGCAC
TCTCTTGGCTGTCACCCGATCGCCAGCAAAACTTAGATGTGAGAAAACCCCTTCCCATTCCATGGCGAAAACATCTCC
TTAGAAAAGCCATTACCCTCATTAGGCATGGTTTTGGGCTCCCAAAACACCTGACAGCCCCTCCCTCCTCTGAGAGGC
GGAGAGTGCTGACTGTAGTGACCATTGCATGCCGGGTGCAGCATCTGGAAGAGCTAGGCAGGGTGTCTGCCCCCTCCT
GAGTTGAAGTCATGCTCCCCTGTGCCAGCCCAGAGGCCGAGAGCTATGGACAGCATTGCCAGTAACACAGGCCACCCT
GTGCAGAAGGGAGCTGGCTCCAGCCTGGAAACCTGTCTGAGGTTGGGAGAGGTGCACTTGGGGCACAGGGAGAGGCCG
GGACACACTTAGCTGGAGATGTCTCTAAAAGCCCTGTATCGTATTCACCTTCAGTTTTTGTGTTTTGGGACAATTACT
TTAGAAAATAAGTAGGTCGTTTTAAAAACAAAAATTATTGATTGCTTTTTTGTAGTGTTCAGAAAAAAGGTTCTTTGT
GTATAGCCAAATGACTGAAAGCACTGATATATTTAAAAACAAAAGGCAATTTATTAAGGAAATTTGTACCATTTCAGT
AAACCTGTCTGAATGTACCTGTATACGTTTCAAAAACACCCCCCCCCCACTGAATCCCTGTAACCTATTTATTATATA
AAGAGTTTGCCTTATAAATTTACATAAAAATGTCCGTTTGTGTCTTTTGTTGTAAAAATCAAGTGATTTTTTCATAAG
GTTCTTTTACTATTGGAAAAGATGGGCAGCACGCAGTTTTATTTTATTTTTGTAAGTTTTTTAATACATGTGAAAGCA
AAGAATACTCAGCATGCCTTTCTAAGTGACGCGTTTGCACCTTTTGTTGGGAAGTACTGTATCCTGTGCTGTTAGCAT
TCTCGATAAATCTCTCTGTGAAAGTGA
SEQ ID NO: 34 >NM_001369394.2 MECP2 [organism = Homo sapiens] [GeneID = 4204]
[transcript = 7]
AGTAGGTGGGGATTATTATCCACAAAAGGGACTAGACATTGTGTTCTGGGTCCCACAACTCATCATAAAGAGGTGGTT
ATAGTTCCCATCAGGAGCCGTGGGTAGGGGACTGTGCGTCCAGCAGCACCCGAGGCTCTTCGGCGCCAGAGGCTCTAA
GGGAAGAAAAGTCAGAAGACCAGGACCTCCAGGGCCTCAAGGACAAACCCCTCAAGTTTAAAAAGGTGAAGAAAGATA
AGAAAGAAGAGAAAGAGGGCAAGCATGAGCCCGTGCAGCCATCAGCCCACCACTCTGCTGAGCCCGCAGAGGCAGGCA
AAGCAGAGACATCAGAAGGGTCAGGCTCCGCCCCGGCTGTGCCGGAAGCTTCTGCCTCCCCCAAACAGCGGCGCTCCA
TCATCCGTGACCGGGGACCCATGTATGATGACCCCACCCTGCCTGAAGGCTGGACACGGAAGCTTAAGCAAAGGAAAT
CTGGCCGCTCTGCTGGGAAGTATGATGTGTATTTGATCAATCCCCAGGGAAAAGCCTTTCGCTCTAAAGTGGAGTTGA
TTGCGTACTTCGAAAAGGTAGGCGACACATCCCTGGACCCTAATGATTTTGACTTCACGGTAACTGGGAGAGGGAGCC
CCTCCCGGCGAGAGCAGAAACCACCTAAGAAGCCCAAATCTCCCAAAGCTCCAGGAACTGGCAGAGGCCGGGGACGCC
CCAAAGGGAGCGGCACCACGAGACCCAAGGCGGCCACGTCAGAGGGTGTGCAGGTGAAAAGGGTCCTGGAGAAAAGTC
CTGGGAAGCTCCTTGTCAAGATGCCTTTTCAAACTTCGCCAGGGGGCAAGGCTGAGGGGGGTGGGGCCACCACATCCA
CCCAGGTCATGGTGATCAAACGCCCCGGCAGGAAGCGAAAAGCTGAGGCCGACCCTCAGGCCATTCCCAAGAAACGGG
GCCGAAAGCCGGGGAGTGTGGTGGCAGCCGCTGCCGCCGAGGCCAAAAAGAAAGCCGTGAAGGAGTCTTCTATCCGAT
CTGTGCAGGAGACCGTACTCCCCATCAAGAAGCGCAAGACCCGGGAGACGGTCAGCATCGAGGTCAAGGAAGTGGTGA
AGCCCCTGCTGGTGTCCACCCTCGGTGAGAAGAGCGGGAAAGGACTGAAGACCTGTAAGAGCCCTGGGCGGAAAAGCA
AGGAGAGCAGCCCCAAGGGGCGCAGCAGCAGCGCCTCCTCACCCCCCAAGAAGGAGCACCACCACCATCACCACCACT
CAGAGTCCCCAAAGGCCCCCGTGCCACTGCTCCCACCCCTGCCCCCACCTCCACCTGAGCCCGAGAGCTCCGAGGACC
CCACCAGCCCCCCTGAGCCCCAGGACTTGAGCAGCAGCGTCTGCAAAGAGGAGAAGATGCCCAGAGGAGGCTCACTGG
AGAGCGACGGCTGCCCCAAGGAGCCAGCTAAGACTCAGCCCGCGGTTGCCACCGCCGCCACGGCCGCAGAAAAGTACA
AACACCGAGGGGAGGGAGAGCGCAAAGACATTGTTTCATCCTCCATGCCAAGGCCAAACAGAGAGGAGCCTGTGGACA
GCCGGACGCCCGTGACCGAGAGAGTTAGCTGACTTTACACGGAGCGGATTGCAAAGCAAACCAACAAGAATAAAGGCA
GCTGTTGTCTCTTCTCCTTATGGGTAGGGCTCTGACAAAGCTTCCCGATTAACTGAAATAAAAAATATTTTTTTTTCT
TTCAGTAAACTTAGAGTTTCGTGGCTTCAGGGTGGGAGTAGTTGGAGCATTGGGGATGTTTTTCTTACCGACAAGCAC
AGTCAGGTTGAAGACCTAACCAGGGCCAGAAGTAGCTTTGCACTTTTCTAAACTAGGCTCCTTCAACAAGGCTTGCTG
CAGATACTACTGACCAGACAAGCTGTTGACCAGGCACCTCCCCTCCCGCCCAAACCTTTCCCCCATGTGGTCGTTAGA
GACAGAGCGACAGAGCAGTTGAGAGGACACTCCCGTTTTCGGTGCCATCAGTGCCCCGTCTACAGCTCCCCCAGCTCC
CCCCACCTCCCCCACTCCCAACCACGTTGGGACAGGGAGGTGTGAGGCAGGAGAGACAGTTGGATTCTTTAGAGAAGA
TGGATATGACCAGTGGCTATGGCCTGTGCGATCCCACCCGTGGTGGCTCAAGTCTGGCCCCACACCAGCCCCAATCCA
AAACTGGCAAGGACGCTTCACAGGACAGGAAAGTGGCACCTGTCTGCTCCAGCTCTGGCATGGCTAGGAGGGGGGAGT
CCCTTGAACTACTGGGTGTAGACTGGCCTGAACCACAGGAGAGGATGGCCCAGGGTGAGGTGGCATGGTCCATTCTCA
AGGGACGTCCTCCAACGGGTGGCGCTAGAGGCCATGGAGGCAGTAGGACAAGGTGCAGGCAGGCTGGCCTGGGGTCAG
GCCGGGCAGAGCACAGCGGGGTGAGAGGGATTCCTAATCACTCAGAGCAGTCTGTGACTTAGTGGACAGGGGAGGGGG
CAAAGGGGGAGGAGAAGAAAATGTTCTTCCAGTTACTTTCCAATTCTCCTTTAGGGACAGCTTAGAATTATTTGCACT
ATTGAGTCTTCATGTTCCCACTTCAAAACAAACAGATGCTCTGAGAGCAAACTGGCTTGAATTGGTGACATTTAGTCC
CTCAAGCCACCAGATGTGACAGTGTTGAGAACTACCTGGATTTGTATATATACCTGCGCTTGTTTTAAAGTGGGCTCA
GCACATAGGGTTCCCACGAAGCTCCGAAACTCTAAGTGTTTGCTGCAATTTTATAAGGACTTCCTGATTGGTTTCTCT
TCTCCCCTTCCATTTCTGCCTTTTGTTCATTTCATCCTTTCACTTCTTTCCCTTCCTCCATCCTCCTCCTTCCTAGTT
CATCCCTTCTCTTCCAGGCAGCCGCGGTGCCCAACCACACTTGTCGGCTCCAGTCCCCAGAACTCTGCCTGCCCTTTG
TCCTCCTGCTGCCAGTACCAGCCCCACCCTGTTTTGAGCCCTGAGGAGGCCTTGGGCTCTGCTGAGTCCGACCTGGCC
TGTCTGTGAAGAGCAAGAGAGCAGCAAGGTCTTGCTCTCCTAGGTAGCCCCCTCTTCCCTGGTAAGAAAAAGCAAAAG
GCATTTCCCACCCTGAACAACGAGCCTTTTCACCCTTCTACTCTAGAGAAGTGGACTGGAGGAGCTGGGCCCGATTTG
GTAGTTGAGGAAAGCACAGAGGCCTCCTGTGGCCTGCCAGTCATCGAGTGGCCCAACAGGGGCTCCATGCCAGCCGAC
CTTGACCTCACTCAGAAGTCCAGAGTCTAGCGTAGTGCAGCAGGGCAGTAGCGGTACCAATGCAGAACTCCCAAGACC
CGAGCTGGGACCAGTACCTGGGTCCCCAGCCCTTCCTCTGCTCCCCCTTTTCCCTCGGAGTTCTTCTTGAATGGCAAT
GTTTTGCTTTTGCTCGATGCAGACAGGGGGCCAGAACACCACACATTTCACTGTCTGTCTGGTCCATAGCTGTGGTGT
AGGGGCTTAGAGGCATGGGCTTGCTGTGGGTTTTTAATTGATCAGTTTTCATGTGGGATCCCATCTTTTTAACCTCTG
TTCAGGAAGTCCTTATCTAGCTGCATATCTTCATCATATTGGTATATCCTTTTCTGTGTTTACAGAGATGTCTCTTAT
ATCTAAATCTGTCCAACTGAGAAGTACCTTATCAAAGTAGCAAATGAGACAGCAGTCTTATGCTTCCAGAAACACCCA
CAGGCATGTCCCATGTGAGCTGCTGCCATGAACTGTCAAGTGTGTGTTGTCTTGTGTATTTCAGTTATTGTCCCTGGC
TTCCTTACTATGGTGTAATCATGAAGGAGTGAAACATCATAGAAACTGTCTAGCACTTCCTTGCCAGTCTTTAGTGAT
CAGGAACCATAGTTGACAGTTCCAATCAGTAGCTTAAGAAAAAACCGTGTTTGTCTCTTCTGGAATGGTTAGAAGTGA
GGGAGTTTGCCCCGTTCTGTTTGTAGAGTCTCATAGTTGGACTTTCTAGCATATATGTGTCCATTTCCTTATGCTGTA
AAAGCAAGTCCTGCAACCAAACTCCCATCAGCCCAATCCCTGATCCCTGATCCCTTCCACCTGCTCTGCTGATGACCC
CCCCAGCTTCACTTCTGACTCTTCCCCAGGAAGGGAAGGGGGGTCAGAAGAGAGGGTGAGTCCTCCAGAACTCTTCCT
CCAAGGACAGAAGGCTCCTGCCCCCATAGTGGCCTCGAACTCCTGGCACTACCAAAGGACACTTATCCACGAGAGCGC
AGCATCCGACCAGGTTGTCACTGAGAAGATGTTTATTTTGGTCAGTTGGGTTTTTATGTATTATACTTAGTCAAATGT
AATGTGGCTTCTGGAATCATTGTCCAGAGCTGCTTCCCCGTCACCTGGGCGTCATCTGGTCCTGGTAAGAGGAGTGCG
TGGCCCACCAGGCCCCCCTGTCACCCATGACAGTTCATTCAGGGCCGATGGGGCAGTCGTGGTTGGGAACACAGCATT
TCAAGCGTCACTTTATTTCATTCGGGCCCCACCTGCAGCTCCCTCAAAGAGGCAGTTGCCCAGCCTCTTTCCCTTCCA
GTTTATTCCAGAGCTGCCAGTGGGGCCTGAGGCTCCTTAGGGTTTTCTCTCTATTTCCCCCTTTCTTCCTCATTCCCT
CGTCTTTCCCAAAGGCATCACGAGTCAGTCGCCTTTCAGCAGGCAGCCTTGGCGGTTTATCGCCCTGGCAGGCAGGGG
CCCTGCAGCTCTCATGCTGCCCCTGCCTTGGGGTCAGGTTGACAGGAGGTTGGAGGGAAAGCCTTAAGCTGCAGGATT
CTCACCAGCTGTGTCCGGCCCAGTTTTGGGGTGTGACCTCAATTTCAATTTTGTCTGTACTTGAACATTATGAAGATG
GGGGCCTCTTTCAGTGAATTTGTGAACAGCAGAATTGACCGACAGCTTTCCAGTACCCATGGGGCTAGGTCATTAAGG
CCACATCCACAGTCTCCCCCACCCTTGTTCCAGTTGTTAGTTACTACCTCCTCTCCTGACAATACTGTATGTCGTCGA
GCTCCCCCCAGGTCTACCCCTCCCGGCCCTGCCTGCTGGTGGGCTTGTCATAGCCAGTGGGATTGCCGGTCTTGACAG
CTCAGTGAGCTGGAGATACTTGGTCACAGCCAGGCGCTAGCACAGCTCCCTTCTGTTGATGCTGTATTCCCATATCAA
AAGACACAGGGGACACCCAGAAACGCCACATCCCCCAATCCATCAGTGCCAAACTAGCCAACGGCCCCAGCTTCTCAG
CTCGCTGGATGGCGGAAGCTGCTACTCGTGAGCGCCAGTGCGGGTGCAGACAATCTTCTGTTGGGTGGCATCATTCCA
GGCCCGAAGCATGAACAGTGCACCTGGGACAGGGAGCAGCCCCAAATTGTCACCTGCTTCTCTGCCCAGCTTTTCATT
GCTGTGACAGTGATGGCGAAAGAGGGTAATAACCAGACACAAACTGCCAAGTTGGGTGGAGAAAGGAGTTTCTTTAGC
TGACAGAATCTCTGAATTTTAAATCACTTAGTAAGCGGCTCAAGCCCAGGAGGGAGCAGAGGGATACGAGCGGAGTCC
CCTGCGCGGGACCATCTGGAATTGGTTTAGCCCAAGTGGAGCCTGACAGCCAGAACTCTGTGTCCCCCGTCTAACCAC
AGCTCCTTTTCCAGAGCATTCCAGTCAGGCTCTCTGGGCTGACTGGGCCAGGGGAGGTTACAGGTACCAGTTCTTTAA
GAAGATCTTTGGGCATATACATTTTTAGCCTGTGTCATTGCCCCAAATGGATTCCTGTTTCAAGTTCACACCTGCAGA
TTCTAGGACCTGTGTCCTAGACTTCAGGGAGTCAGCTGTTTCTAGAGTTCCTACCATGGAGTGGGTCTGGAGGACCTG
CCCGGTGGGGGGGCAGAGCCCTGCTCCCTCCGGGTCTTCCTACTCTTCTCTCTGCTCTGACGGGATTTGTTGATTCTC
TCCATTTTGGTGTCTTTCTCTTTTAGATATTGTATCAATCTTTAGAAAAGGCATAGTCTACTTGTTATAAATCGTTAG
GATACTGCCTCCCCCAGGGTCTAAAATTACATATTAGAGGGGAAAAGCTGAACACTGAAGTCAGTTCTCAACAATTTA
GAAGGAAAACCTAGAAAACATTTGGCAGAAAATTACATTTCGATGTTTTTGAATGAATACGAGCAAGCTTTTACAACA
GTGCTGATCTAAAAATACTTAGCACTTGGCCTGAGATGCCTGGTGAGCATTACAGGCAAGGGGAATCTGGAGGTAGCC
GACCTGAGGACATGGCTTCTGAACCTGTCTTTTGGGAGTGGTATGGAAGGTGGAGCGTTCACCAGTGACCTGGAAGGC
CCAGCACCACCCTCCTTCCCACTCTTCTCATCTTGACAGAGCCTGCCCCAGCGCTGACGTGTCAGGAAAACACCCAGG
GAACTAGGAAGGCACTTCTGCCTGAGGGGCAGCCTGCCTTGCCCACTCCTGCTCTGCTCGCCTCGGATCAGCTGAGCC
TTCTGAGCTGGCCTCTCACTGCCTCCCCAAGGCCCCCTGCCTGCCCTGTCAGGAGGCAGAAGGAAGCAGGTGTGAGGG
CAGTGCAAGGAGGGAGCACAACCCCCAGCTCCCGCTCCGGGCTCCGACTTGTGCACAGGCAGAGCCCAGACCCTGGAG
GAAATCCTACCTTTGAATTCAAGAACATTTGGGGAATTTGGAAATCTCTTTGCCCCCAAACCCCCATTCTGTCCTACC
TTTAATCAGGTCCTGCTCAGCAGTGAGAGCAGATGAGGTGAAAAGGCCAAGAGGTTTGGCTCCTGCCCACTGATAGCC
CCTCTCCCCGCAGTGTTTGTGTGTCAAGTGGCAAAGCTGTTCTTCCTGGTGACCCTGATTATATCCAGTAACACATAG
ACTGTGCGCATAGGCCTGCTTTGTCTCCTCTATCCTGGGCTTTTGTTTTGCTTTTTAGTTTTGCTTTTAGTTTTTCTG
TCCCTTTTATTTAACGCACCGACTAGACACACAAAGCAGTTGAATTTTTATATATATATCTGTATATTGCACAATTAT
AAACTCATTTTGCTTGTGGCTCCACACACACAAAAAAAGACCTGTTAAAATTATACCTGTTGCTTAATTACAATATTT
CTGATAACCATAGCATAGGACAAGGGAAAATAAAAAAAGAAAAAAAAGAAAAAAAAACGACAAATCTGTCTGCTGGTC
ACTTCTTCTGTCCAAGCAGATTCGTGGTCTTTTCCTCGCTTCTTTCAAGGGCTTTCCTGTGCCAGGTGAAGGAGGCTC
CAGGCAGCACCCAGGTTTTGCACTCTTGTTTCTCCCGTGCTTGTGAAAGAGGTCCCAAGGTTCTGGGTGCAGGAGCGC
TCCCTTGACCTGCTGAAGTCCGGAACGTAGTCGGCACAGCCTGGTCGCCTTCCACCTCTGGGAGCTGGAGTCCACTGG
GGTGGCCTGACTCCCCCAGTCCCCTTCCCGTGACCTGGTCAGGGTGAGCCCATGTGGAGTCAGCCTCGCAGGCCTCCC
TGCCAGTAGGGTCCGAGTGTGTTTCATCCTTCCCACTCTGTCGAGCCTGGGGGCTGGAGCGGAGACGGGAGGCCTGGC
CTGTCTCGGAACCTGTGAGCTGCACCAGGTAGAACGCCAGGGACCCCAGAATCATGTGCGTCAGTCCAAGGGGTCCCC
TCCAGGAGTAGTGAAGACTCCAGAAATGTCCCTTTCTTCTCCCCCATCCTACGAGTAATTGCATTTGCTTTTGTAATT
CTTAATGAGCAATATCTGCTAGAGAGTTTAGCTGTAACAGTTCTTTTTGATCATCTTTTTTTAATAATTAGAAACACC
AAAAAAATCCAGAAACTTGTTCTTCCAAAGCAGAGAGCATTATAATCACCAGGGCCAAAAGCTTCCCTCCCTGCTGTC
ATTGCTTCTTCTGAGGCCTGAATCCAAAAGAAAAACAGCCATAGGCCCTTTCAGTGGCCGGGCTACCCGTGAGCCCTT
CGGAGGACCAGGGCTGGGGCAGCCTCTGGGCCCACATCCGGGGCCAGCTCCGGCGTGTGTTCAGTGTTAGCAGTGGGT
CATGATGCTCTTTCCCACCCAGCCTGGGATAGGGGCAGAGGAGGCGAGGAGGCCGTTGCCGCTGATGTTTGGCCGTGA
ACAGGTGGGTGTCTGCGTGCGTCCACGTGCGTGTTTTCTGACTGACATGAAATCGACGCCCGAGTTAGCCTCACCCGG
TGACCTCTAGCCCTGCCCGGATGGAGCGGGGCCCACCCGGTTCAGTGTTTCTGGGGAGCTGGACAGTGGAGTGCAAAA
GGCTTGCAGAACTTGAAGCCTGCTCCTTCCCTTGCTACCACGGCCTCCTTTCCGTTTGATTTGTCACTGCTTCAATCA
ATAACAGCCGCTCCAGAGTCAGTAGTCAATGAATATATGACCAAATATCACCAGGACTGTTACTCAATGTGTGCCGAG
CCCTTGCCCATGCTGGGCTCCCGTGTATCTGGACACTGTAACGTGTGCTGTGTTTGCTCCCCTTCCCCTTCCTTCTTT
GCCCTTTACTTGTCTTTCTGGGGTTTTTCTGTTTGGGTTTGGTTTGGTTTTTATTTCTCCTTTTGTGTTCCAAACATG
AGGTTCTCTCTACTGGTCCTCTTAACTGTGGTGTTGAGGCTTATATTTGTGTAATTTTTGGTGGGTGAAAGGAATTTT
GCTAAGTAAATCTCTTCTGTGTTTGAACTGAAGTCTGTATTGTAACTATGTTTAAAGTAATTGTTCCAGAGACAAATA
TTTCTAGACACTTTTTCTTTACAAACAAAAGCATTCGGAGGGAGGGGGATGGTGACTGAGATGAGAGGGGAGAGCTGA
ACAGATGACCCCTGCCCAGATCAGCCAGAAGCCACCCAAAGCAGTGGAGCCCAGGAGTCCCACTCCAAGCCAGCAAGC
CGAATAGCTGATGTGTTGCCACTTTCCAAGTCACTGCAAAACCAGGTTTTGTTCCGCCCAGTGGATTCTTGTTTTGCT
TCCCCTCCCCCCGAGATTATTACCACCATCCCGTGCTTTTAAGGAAAGGCAAGATTGATGTTTCCTTGAGGGGAGCCA
GGAGGGGATGTGTGTGTGCAGAGCTGAAGAGCTGGGGAGAATGGGGCTGGGCCCACCCAAGCAGGAGGCTGGGACGCT
CTGCTGTGGGCACAGGTCAGGCTAATGTTGGCAGATGCAGCTCTTCCTGGACAGGCCAGGTGGTGGGCATTCTCTCTC
CAAGGTGTGCCCCGTGGGCATTACTGTTTAAGACACTTCCGTCACATCCCACCCCATCCTCCAGGGCTCAACACTGTG
ACATCTCTATTCCCCACCCTCCCCTTCCCAGGGCAATAAAATGACCATGGAGGGGGCTTGCACTCTCTTGGCTGTCAC
CCGATCGCCAGCAAAACTTAGATGTGAGAAAACCCCTTCCCATTCCATGGCGAAAACATCTCCTTAGAAAAGCCATTA
CCCTCATTAGGCATGGTTTTGGGCTCCCAAAACACCTGACAGCCCCTCCCTCCTCTGAGAGGCGGAGAGTGCTGACTG
TAGTGACCATTGCATGCCGGGTGCAGCATCTGGAAGAGCTAGGCAGGGTGTCTGCCCCCTCCTGAGTTGAAGTCATGC
TCCCCTGTGCCAGCCCAGAGGCCGAGAGCTATGGACAGCATTGCCAGTAACACAGGCCACCCTGTGCAGAAGGGAGCT
GGCTCCAGCCTGGAAACCTGTCTGAGGTTGGGAGAGGTGCACTTGGGGCACAGGGAGAGGCCGGGACACACTTAGCTG
GAGATGTCTCTAAAAGCCCTGTATCGTATTCACCTTCAGTTTTTGTGTTTTGGGACAATTACTTTAGAAAATAAGTAG
GTCGTTTTAAAAACAAAAATTATTGATTGCTTTTTTGTAGTGTTCAGAAAAAAGGTTCTTTGTGTATAGCCAAATGAC
TGAAAGCACTGATATATTTAAAAACAAAAGGCAATTTATTAAGGAAATTTGTACCATTTCAGTAAACCTGTCTGAATG
TACCTGTATACGTTTCAAAAACACCCCCCCCCCACTGAATCCCTGTAACCTATTTATTATATAAAGAGTTTGCCTTAT
AAATTTACATAAAAATGTCCGTTTGTGTCTTTTGTTGTAAAAATCAAGTGATTTTTTCATAAGGTTCTTTTACTATTG
GAAAAGATGGGCAGCACGCAGTTTTATTTTATTTTTGTAAGTTTTTTAATACATGTGAAAGCAAAGAATACTCAGCAT
GCCTTTCTAAGTGACGCGTTTGCACCTTTTGTTGGGAAGTACTGTATCCTGTGCTGTTAGCATTCTCGATAAATCTCT
CTGTGAAAGTGA
SEQ ID NO: 35 >XM_024452383.1 MECP2 [organism = Homo sapiens] [GeneID = 4204]
[transcript = X1]
TAAAAATACAGACTCACCAGTTCCTGCTTTGATGTGACATGTGACTCCCCAGAATACACCTTGCTTCTGTAGACCAGC
TCCAACAGGATTCCATGGTAGCTGGGATGTTAGGGCTCAGGACCCAGGAGGGAGAAGCAGCTGCCAGGGGAAGTCTCT
TCGTAGGCGGAGGTCAGGAGTCCAAGAGGAGTGAGCAGAGTCACAGAAGCCTCTTAAAGCCTCTTCTTCCCCCATCCC
ATCAACACATCTGCTGCCCAAGAACTCCTTTGTGATTGAATTTAACCAGAGTTGATTTCTGTTGCTTGAAACTCAGGA
CCTTGATGCAGTGTGCAGGGTCTGGGCAAGTCTAGACTTGAATGCAAACAGTTCTTATTGCTAACTTACCACTAGTCA
CCACTAATGAACAAAGACTATGAACAGGAAATTCATAAAAGAAGAGATACAGATGGCCAATGAAGATAGGAAAAGAGT
TCTGCCTGCTGGTAATCAAAGAGATGCAAACGAGAACAAAAATGATGCCTTTTCACCTACCAAATTTGTCAAGATTAA
AAAGAAAGCAAAGAGCCAGCGTCAGCTGATGTTCATACCTGCACCTGCTCGGTAGCTTGCTAATGTTCTGCCTGCTCC
ACACGCCAGGCCAGCCTCCACGGCGCAGCCAGGGAAGAAAAGTCAGAAGACCAGGACCTCCAGGGCCTCAAGGACAAA
CCCCTCAAGTTTAAAAAGGTGAAGAAAGATAAGAAAGAAGAGAAAGAGGGCAAGCATGAGCCCGTGCAGCCATCAGCC
CACCACTCTGCTGAGCCCGCAGAGGCAGGCAAAGCAGAGACATCAGAAGGGTCAGGCTCCGCCCCGGCTGTGCCGGAA
GCTTCTGCCTCCCCCAAACAGCGGCGCTCCATCATCCGTGACCGGGGACCCATGTATGATGACCCCACCCTGCCTGAA
GGCTGGACACGGAAGCTTAAGCAAAGGAAATCTGGCCGCTCTGCTGGGAAGTATGATGTGTATTTGATCAATCCCCAG
GGAAAAGCCTTTCGCTCTAAAGTGGAGTTGATTGCGTACTTCGAAAAGGTAGGCGACACATCCCTGGACCCTAATGAT
TTTGACTTCACGGTAACTGGGAGAGGGAGCCCCTCCCGGCGAGAGCAGAAACCACCTAAGAAGCCCAAATCTCCCAAA
GCTCCAGGAACTGGCAGAGGCCGGGGACGCCCCAAAGGGAGCGGCACCACGAGACCCAAGGCGGCCACGTCAGAGGGT
GTGCAGGTGAAAAGGGTCCTGGAGAAAAGTCCTGGGAAGCTCCTTGTCAAGATGCCTTTTCAAACTTCGCCAGGGGGC
AAGGCTGAGGGGGGTGGGGCCACCACATCCACCCAGGTCATGGTGATCAAACGCCCCGGCAGGAAGCGAAAAGCTGAG
GCCGACCCTCAGGCCATTCCCAAGAAACGGGGCCGAAAGCCGGGGAGTGTGGTGGCAGCCGCTGCCGCCGAGGCCAAA
AAGAAAGCCGTGAAGGAGTCTTCTATCCGATCTGTGCAGGAGACCGTACTCCCCATCAAGAAGCGCAAGACCCGGGAG
ACGGTCAGCATCGAGGTCAAGGAAGTGGTGAAGCCCCTGCTGGTGTCCACCCTCGGTGAGAAGAGCGGGAAAGGACTG
AAGACCTGTAAGAGCCCTGGGCGGAAAAGCAAGGAGAGCAGCCCCAAGGGGCGCAGCAGCAGCGCCTCCTCACCCCCC
AAGAAGGAGCACCACCACCATCACCACCACTCAGAGTCCCCAAAGGCCCCCGTGCCACTGCTCCCACCCCTGCCCCCA
CCTCCACCTGAGCCCGAGAGCTCCGAGGACCCCACCAGCCCCCCTGAGCCCCAGGACTTGAGCAGCAGCGTCTGCAAA
GAGGAGAAGATGCCCAGAGGAGGCTCACTGGAGAGCGACGGCTGCCCCAAGGAGCCAGCTAAGACTCAGCCCGCGGTT
GCCACCGCCGCCACGGCCGCAGAAAAGTACAAACACCGAGGGGAGGGAGAGCGCAAAGACATTGTTTCATCCTCCATG
CCAAGGCCAAACAGAGAGGAGCCTGTGGACAGCCGGACGCCCGTGACCGAGAGAGTTAGCTGACTTTACACGGAGCGG
ATTGCAAAGCAAACCAACAAGAATAAAGGCAGCTGTTGTCTCTTCTCCTTATGGGTAGGGCTCTGACAAAGCTTCCCG
ATTAACTGAAATAAAAAATATTTTTTTTTCTTTCAGTAAACTTAGAGTTTCGTGGCTTCAGGGTGGGAGTAGTTGGAG
CATTGGGGATGTTTTTCTTACCGACAAGCACAGTCAGGTTGAAGACCTAACCAGGGCCAGAAGTAGCTTTGCACTTTT
CTAAACTAGGCTCCTTCAACAAGGCTTGCTGCAGATACTACTGACCAGACAAGCTGTTGACCAGGCACCTCCCCTCCC
GCCCAAACCTTTCCCCCATGTGGTCGTTAGAGACAGAGCGACAGAGCAGTTGAGAGGACACTCCCGTTTTCGGTGCCA
TCAGTGCCCCGTCTACAGCTCCCCCAGCTCCCCCCACCTCCCCCACTCCCAACCACGTTGGGACAGGGAGGTGTGAGG
CAGGAGAGACAGTTGGATTCTTTAGAGAAGATGGATATGACCAGTGGCTATGGCCTGTGCGATCCCACCCGTGGTGGC
TCAAGTCTGGCCCCACACCAGCCCCAATCCAAAACTGGCAAGGACGCTTCACAGGACAGGAAAGTGGCACCTGTCTGC
TCCAGCTCTGGCATGGCTAGGAGGGGGGAGTCCCTTGAACTACTGGGTGTAGACTGGCCTGAACCACAGGAGAGGATG
GCCCAGGGTGAGGTGGCATGGTCCATTCTCAAGGGACGTCCTCCAACGGGTGGCGCTAGAGGCCATGGAGGCAGTAGG
ACAAGGTGCAGGCAGGCTGGCCTGGGGTCAGGCCGGGCAGAGCACAGCGGGGTGAGAGGGATTCCTAATCACTCAGAG
CAGTCTGTGACTTAGTGGACAGGGGAGGGGGCAAAGGGGGAGGAGAAGAAAATGTTCTTCCAGTTACTTTCCAATTCT
CCTTTAGGGACAGCTTAGAATTATTTGCACTATTGAGTCTTCATGTTCCCACTTCAAAACAAACAGATGCTCTGAGAG
CAAACTGGCTTGAATTGGTGACATTTAGTCCCTCAAGCCACCAGATGTGACAGTGTTGAGAACTACCTGGATTTGTAT
ATATACCTGCGCTTGTTTTAAAGTGGGCTCAGCACATAGGGTTCCCACGAAGCTCCGAAACTCTAAGTGTTTGCTGCA
ATTTTATAAGGACTTCCTGATTGGTTTCTCTTCTCCCCTTCCATTTCTGCCTTTTGTTCATTTCATCCTTTCACTTCT
TTCCCTTCCTCCATCCTCCTCCTTCCTAGTTCATCCCTTCTCTTCCAGGCAGCCGCGGTGCCCAACCACACTTGTCGG
CTCCAGTCCCCAGAACTCTGCCTGCCCTTTGTCCTCCTGCTGCCAGTACCAGCCCCACCCTGTTTTGAGCCCTGAGGA
GGCCTTGGGCTCTGCTGAGTCCGACCTGGCCTGTCTGTGAAGAGCAAGAGAGCAGCAAGGTCTTGCTCTCCTAGGTAG
CCCCCTCTTCCCTGGTAAGAAAAAGCAAAAGGCATTTCCCACCCTGAACAACGAGCCTTTTCACCCTTCTACTCTAGA
GAAGTGGACTGGAGGAGCTGGGCCCGATTTGGTAGTTGAGGAAAGCACAGAGGCCTCCTGTGGCCTGCCAGTCATCGA
GTGGCCCAACAGGGGCTCCATGCCAGCCGACCTTGACCTCACTCAGAAGTCCAGAGTCTAGCGTAGTGCAGCAGGGCA
GTAGCGGTACCAATGCAGAACTCCCAAGACCCGAGCTGGGACCAGTACCTGGGTCCCCAGCCCTTCCTCTGCTCCCCC
TTTTCCCTCGGAGTTCTTCTTGAATGGCAATGTTTTGCTTTTGCTCGATGCAGACAGGGGGCCAGAACACCACACATT
TCACTGTCTGTCTGGTCCATAGCTGTGGTGTAGGGGCTTAGAGGCATGGGCTTGCTGTGGGTTTTTAATTGATCAGTT
TTCATGTGGGATCCCATCTTTTTAACCTCTGTTCAGGAAGTCCTTATCTAGCTGCATATCTTCATCATATTGGTATAT
CCTTTTCTGTGTTTACAGAGATGTCTCTTATATCTAAATCTGTCCAACTGAGAAGTACCTTATCAAAGTAGCAAATGA
GACAGCAGTCTTATGCTTCCAGAAACACCCACAGGCATGTCCCATGTGAGCTGCTGCCATGAACTGTCAAGTGTGTGT
TGTCTTGTGTATTTCAGTTATTGTCCCTGGCTTCCTTACTATGGTGTAATCATGAAGGAGTGAAACATCATAGAAACT
GTCTAGCACTTCCTTGCCAGTCTTTAGTGATCAGGAACCATAGTTGACAGTTCCAATCAGTAGCTTAAGAAAAAACCG
TGTTTGTCTCTTCTGGAATGGTTAGAAGTGAGGGAGTTTGCCCCGTTCTGTTTGTAGAGTCTCATAGTTGGACTTTCT
AGCATATATGTGTCCATTTCCTTATGCTGTAAAAGCAAGTCCTGCAACCAAACTCCCATCAGCCCAATCCCTGATCCC
TGATCCCTTCCACCTGCTCTGCTGATGACCCCCCCAGCTTCACTTCTGACTCTTCCCCAGGAAGGGAAGGGGGGTCAG
AAGAGAGGGTGAGTCCTCCAGAACTCTTCCTCCAAGGACAGAAGGCTCCTGCCCCCATAGTGGCCTCGAACTCCTGGC
ACTACCAAAGGACACTTATCCACGAGAGCGCAGCATCCGACCAGGTTGTCACTGAGAAGATGTTTATTTTGGTCAGTT
GGGTTTTTATGTATTATACTTAGTCAAATGTAATGTGGCTTCTGGAATCATTGTCCAGAGCTGCTTCCCCGTCACCTG
GGCGTCATCTGGTCCTGGTAAGAGGAGTGCGTGGCCCACCAGGCCCCCCTGTCACCCATGACAGTTCATTCAGGGCCG
ATGGGGCAGTCGTGGTTGGGAACACAGCATTTCAAGCGTCACTTTATTTCATTCGGGCCCCACCTGCAGCTCCCTCAA
AGAGGCAGTTGCCCAGCCTCTTTCCCTTCCAGTTTATTCCAGAGCTGCCAGTGGGGCCTGAGGCTCCTTAGGGTTTTC
TCTCTATTTCCCCCTTTCTTCCTCATTCCCTCGTCTTTCCCAAAGGCATCACGAGTCAGTCGCCTTTCAGCAGGCAGC
CTTGGCGGTTTATCGCCCTGGCAGGCAGGGGCCCTGCAGCTCTCATGCTGCCCCTGCCTTGGGGTCAGGTTGACAGGA
GGTTGGAGGGAAAGCCTTAAGCTGCAGGATTCTCACCAGCTGTGTCCGGCCCAGTTTTGGGGTGTGACCTCAATTTCA
ATTTTGTCTGTACTTGAACATTATGAAGATGGGGGCCTCTTTCAGTGAATTTGTGAACAGCAGAATTGACCGACAGCT
TTCCAGTACCCATGGGGCTAGGTCATTAAGGCCACATCCACAGTCTCCCCCACCCTTGTTCCAGTTGTTAGTTACTAC
CTCCTCTCCTGACAATACTGTATGTCGTCGAGCTCCCCCCAGGTCTACCCCTCCCGGCCCTGCCTGCTGGTGGGCTTG
TCATAGCCAGTGGGATTGCCGGTCTTGACAGCTCAGTGAGCTGGAGATACTTGGTCACAGCCAGGCGCTAGCACAGCT
CCCTTCTGTTGATGCTGTATTCCCATATCAAAAGACACAGGGGACACCCAGAAACGCCACATCCCCCAATCCATCAGT
GCCAAACTAGCCAACGGCCCCAGCTTCTCAGCTCGCTGGATGGCGGAAGCTGCTACTCGTGAGCGCCAGTGCGGGTGC
AGACAATCTTCTGTTGGGTGGCATCATTCCAGGCCCGAAGCATGAACAGTGCACCTGGGACAGGGAGCAGCCCCAAAT
TGTCACCTGCTTCTCTGCCCAGCTTTTCATTGCTGTGACAGTGATGGCGAAAGAGGGTAATAACCAGACACAAACTGC
CAAGTTGGGTGGAGAAAGGAGTTTCTTTAGCTGACAGAATCTCTGAATTTTAAATCACTTAGTAAGCGGCTCAAGCCC
AGGAGGGAGCAGAGGGATACGAGCGGAGTCCCCTGCGCGGGACCATCTGGAATTGGTTTAGCCCAAGTGGAGCCTGAC
AGCCAGAACTCTGTGTCCCCCGTCTAACCACAGCTCCTTTTCCAGAGCATTCCAGTCAGGCTCTCTGGGCTGACTGGG
CCAGGGGAGGTTACAGGTACCAGTTCTTTAAGAAGATCTTTGGGCATATACATTTTTAGCCTGTGTCATTGCCCCAAA
TGGATTCCTGTTTCAAGTTCACACCTGCAGATTCTAGGACCTGTGTCCTAGACTTCAGGGAGTCAGCTGTTTCTAGAG
TTCCTACCATGGAGTGGGTCTGGAGGACCTGCCCGGTGGGGGGGCAGAGCCCTGCTCCCTCCGGGTCTTCCTACTCTT
CTCTCTGCTCTGACGGGATTTGTTGATTCTCTCCATTTTGGTGTCTTTCTCTTTTAGATATTGTATCAATCTTTAGAA
AAGGCATAGTCTACTTGTTATAAATCGTTAGGATACTGCCTCCCCCAGGGTCTAAAATTACATATTAGAGGGGAAAAG
CTGAACACTGAAGTCAGTTCTCAACAATTTAGAAGGAAAACCTAGAAAACATTTGGCAGAAAATTACATTTCGATGTT
TTTGAATGAATACGAGCAAGCTTTTACAACAGTGCTGATCTAAAAATACTTAGCACTTGGCCTGAGATGCCTGGTGAG
CATTACAGGCAAGGGGAATCTGGAGGTAGCCGACCTGAGGACATGGCTTCTGAACCTGTCTTTTGGGAGTGGTATGGA
AGGTGGAGCGTTCACCAGTGACCTGGAAGGCCCAGCACCACCCTCCTTCCCACTCTTCTCATCTTGACAGAGCCTGCC
CCAGCGCTGACGTGTCAGGAAAACACCCAGGGAACTAGGAAGGCACTTCTGCCTGAGGGGCAGCCTGCCTTGCCCACT
CCTGCTCTGCTCGCCTCGGATCAGCTGAGCCTTCTGAGCTGGCCTCTCACTGCCTCCCCAAGGCCCCCTGCCTGCCCT
GTCAGGAGGCAGAAGGAAGCAGGTGTGAGGGCAGTGCAAGGAGGGAGCACAACCCCCAGCTCCCGCTCCGGGCTCCGA
CTTGTGCACAGGCAGAGCCCAGACCCTGGAGGAAATCCTACCTTTGAATTCAAGAACATTTGGGGAATTTGGAAATCT
CTTTGCCCCCAAACCCCCATTCTGTCCTACCTTTAATCAGGTCCTGCTCAGCAGTGAGAGCAGATGAGGTGAAAAGGC
CAAGAGGTTTGGCTCCTGCCCACTGATAGCCCCTCTCCCCGCAGTGTTTGTGTGTCAAGTGGCAAAGCTGTTCTTCCT
GGTGACCCTGATTATATCCAGTAACACATAGACTGTGCGCATAGGCCTGCTTTGTCTCCTCTATCCTGGGCTTTTGTT
TTGCTTTTTAGTTTTGCTTTTAGTTTTTCTGTCCCTTTTATTTAACGCACCGACTAGACACACAAAGCAGTTGAATTT
TTATATATATATCTGTATATTGCACAATTATAAACTCATTTTGCTTGTGGCTCCACACACACAAAAAAAGACCTGTTA
AAATTATACCTGTTGCTTAATTACAATATTTCTGATAACCATAGCATAGGACAAGGGAAAATAAAAAAAGAAAAAAAA
GAAAAAAAAACGACAAATCTGTCTGCTGGTCACTTCTTCTGTCCAAGCAGATTCGTGGTCTTTTCCTCGCTTCTTTCA
AGGGCTTTCCTGTGCCAGGTGAAGGAGGCTCCAGGCAGCACCCAGGTTTTGCACTCTTGTTTCTCCCGTGCTTGTGAA
AGAGGTCCCAAGGTTCTGGGTGCAGGAGCGCTCCCTTGACCTGCTGAAGTCCGGAACGTAGTCGGCACAGCCTGGTCG
CCTTCCACCTCTGGGAGCTGGAGTCCACTGGGGTGGCCTGACTCCCCCAGTCCCCTTCCCGTGACCTGGTCAGGGTGA
GCCCATGTGGAGTCAGCCTCGCAGGCCTCCCTGCCAGTAGGGTCCGAGTGTGTTTCATCCTTCCCACTCTGTCGAGCC
TGGGGGCTGGAGCGGAGACGGGAGGCCTGGCCTGTCTCGGAACCTGTGAGCTGCACCAGGTAGAACGCCAGGGACCCC
AGAATCATGTGCGTCAGTCCAAGGGGTCCCCTCCAGGAGTAGTGAAGACTCCAGAAATGTCCCTTTCTTCTCCCCCAT
CCTACGAGTAATTGCATTTGCTTTTGTAATTCTTAATGAGCAATATCTGCTAGAGAGTTTAGCTGTAACAGTTCTTTT
TGATCATCTTTTTTTAATAATTAGAAACACCAAAAAAATCCAGAAACTTGTTCTTCCAAAGCAGAGAGCATTATAATC
ACCAGGGCCAAAAGCTTCCCTCCCTGCTGTCATTGCTTCTTCTGAGGCCTGAATCCAAAAGAAAAACAGCCATAGGCC
CTTTCAGTGGCCGGGCTACCCGTGAGCCCTTCGGAGGACCAGGGCTGGGGCAGCCTCTGGGCCCACATCCGGGGCCAG
CTCCGGCGTGTGTTCAGTGTTAGCAGTGGGTCATGATGCTCTTTCCCACCCAGCCTGGGATAGGGGCAGAGGAGGCGA
GGAGGCCGTTGCCGCTGATGTTTGGCCGTGAACAGGTGGGTGTCTGCGTGCGTCCACGTGCGTGTTTTCTGACTGACA
TGAAATCGACGCCCGAGTTAGCCTCACCCGGTGACCTCTAGCCCTGCCCGGATGGAGCGGGGCCCACCCGGTTCAGTG
TTTCTGGGGAGCTGGACAGTGGAGTGCAAAAGGCTTGCAGAACTTGAAGCCTGCTCCTTCCCTTGCTACCACGGCCTC
CTTTCCGTTTGATTTGTCACTGCTTCAATCAATAACAGCCGCTCCAGAGTCAGTAGTCAATGAATATATGACCAAATA
TCACCAGGACTGTTACTCAATGTGTGCCGAGCCCTTGCCCATGCTGGGCTCCCGTGTATCTGGACACTGTAACGTGTG
CTGTGTTTGCTCCCCTTCCCCTTCCTTCTTTGCCCTTTACTTGTCTTTCTGGGGTTTTTCTGTTTGGGTTTGGTTTGG
TTTTTATTTCTCCTTTTGTGTTCCAAACATGAGGTTCTCTCTACTGGTCCTCTTAACTGTGGTGTTGAGGCTTATATT
TGTGTAATTTTTGGTGGGTGAAAGGAATTTTGCTAAGTAAATCTCTTCTGTGTTTGAACTGAAGTCTGTATTGTAACT
ATGTTTAAAGTAATTGTTCCAGAGACAAATATTTCTAGACACTTTTTCTTTACAAACAAAAGCATTCGGAGGGAGGGG
GATGGTGACTGAGATGAGAGGGGAGAGCTGAACAGATGACCCCTGCCCAGATCAGCCAGAAGCCACCCAAAGCAGTGG
AGCCCAGGAGTCCCACTCCAAGCCAGCAAGCCGAATAGCTGATGTGTTGCCACTTTCCAAGTCACTGCAAAACCAGGT
TTTGTTCCGCCCAGTGGATTCTTGTTTTGCTTCCCCTCCCCCCGAGATTATTACCACCATCCCGTGCTTTTAAGGAAA
GGCAAGATTGATGTTTCCTTGAGGGGAGCCAGGAGGGGATGTGTGTGTGCAGAGCTGAAGAGCTGGGGAGAATGGGGC
TGGGCCCACCCAAGCAGGAGGCTGGGACGCTCTGCTGTGGGCACAGGTCAGGCTAATGTTGGCAGATGCAGCTCTTCC
TGGACAGGCCAGGTGGTGGGCATTCTCTCTCCAAGGTGTGCCCCGTGGGCATTACTGTTTAAGACACTTCCGTCACAT
CCCACCCCATCCTCCAGGGCTCAACACTGTGACATCTCTATTCCCCACCCTCCCCTTCCCAGGGCAATAAAATGACCA
TGGAGGGGGCTTGCACTCTCTTGGCTGTCACCCGATCGCCAGCAAAACTTAGATGTGAGAAAACCCCTTCCCATTCCA
TGGCGAAAACATCTCCTTAGAAAAGCCATTACCCTCATTAGGCATGGTTTTGGGCTCCCAAAACACCTGACAGCCCCT
CCCTCCTCTGAGAGGCGGAGAGTGCTGACTGTAGTGACCATTGCATGCCGGGTGCAGCATCTGGAAGAGCTAGGCAGG
GTGTCTGCCCCCTCCTGAGTTGAAGTCATGCTCCCCTGTGCCAGCCCAGAGGCCGAGAGCTATGGACAGCATTGCCAG
TAACACAGGCCACCCTGTGCAGAAGGGAGCTGGCTCCAGCCTGGAAACCTGTCTGAGGTTGGGAGAGGTGCACTTGGG
GCACAGGGAGAGGCCGGGACACACTTAGCTGGAGATGTCTCTAAAAGCCCTGTATCGTATTCACCTTCAGTTTTTGTG
TTTTGGGACAATTACTTTAGAAAATAAGTAGGTCGTTTTAAAAACAAAAATTATTGATTGCTTTTTTGTAGTGTTCAG
AAAAAAGGTTCTTTGTGTATAGCCAAATGACTGAAAGCACTGATATATTTAAAAACAAAAGGCAATTTATTAAGGAAA
TTTGTACCATTTCAGTAAACCTGTCTGAATGTACCTGTATACGTTTCAAAAACACCCCCCCCCCACTGAATCCCTGTA
ACCTATTTATTATATAAAGAGTTTGCCTTATAAATTTACATAAAAATGTC
SEQ ID NO: 36 >XM_011531166.2 MECP2 [organism = Homo sapiens] [GeneID = 4204]
[transcript = X3]
GCAGTAGTCTCACGTGGCAGACCCGGAGGTCACTCTCCTCTCTGGATGCCCTGCGGACACTCCGGTTAGCCTCAGCTG
CAAGAGCCTCCTCACCCAAGGTCACGCCATTTCCAGGACAGCACCCTGGTGACTGAGCGAGGGAAGAAAAGTCAGAAG
ACCAGGACCTCCAGGGCCTCAAGGACAAACCCCTCAAGTTTAAAAAGGTGAAGAAAGATAAGAAAGAAGAGAAAGAGG
GCAAGCATGAGCCCGTGCAGCCATCAGCCCACCACTCTGCTGAGCCCGCAGAGGCAGGCAAAGCAGAGACATCAGAAG
GGTCAGGCTCCGCCCCGGCTGTGCCGGAAGCTTCTGCCTCCCCCAAACAGCGGCGCTCCATCATCCGTGACCGGGGAC
CCATGTATGATGACCCCACCCTGCCTGAAGGCTGGACACGGAAGCTTAAGCAAAGGAAATCTGGCCGCTCTGCTGGGA
AGTATGATGTGTATTTGATCAATCCCCAGGGAAAAGCCTTTCGCTCTAAAGTGGAGTTGATTGCGTACTTCGAAAAGG
TAGGCGACACATCCCTGGACCCTAATGATTTTGACTTCACGGTAACTGGGAGAGGGAGCCCCTCCCGGCGAGAGCAGA
AACCACCTAAGAAGCCCAAATCTCCCAAAGCTCCAGGAACTGGCAGAGGCCGGGGACGCCCCAAAGGGAGCGGCACCA
CGAGACCCAAGGCGGCCACGTCAGAGGGTGTGCAGGTGAAAAGGGTCCTGGAGAAAAGTCCTGGGAAGCTCCTTGTCA
AGATGCCTTTTCAAACTTCGCCAGGGGGCAAGGCTGAGGGGGGTGGGGCCACCACATCCACCCAGGTCATGGTGATCA
AACGCCCCGGCAGGAAGCGAAAAGCTGAGGCCGACCCTCAGGCCATTCCCAAGAAACGGGGCCGAAAGCCGGGGAGTG
TGGTGGCAGCCGCTGCCGCCGAGGCCAAAAAGAAAGCCGTGAAGGAGTCTTCTATCCGATCTGTGCAGGAGACCGTAC
TCCCCATCAAGAAGCGCAAGACCCGGGAGACGGTCAGCATCGAGGTCAAGGAAGTGGTGAAGCCCCTGCTGGTGTCCA
CCCTCGGTGAGAAGAGCGGGAAAGGACTGAAGACCTGTAAGAGCCCTGGGCGGAAAAGCAAGGAGAGCAGCCCCAAGG
GGCGCAGCAGCAGCGCCTCCTCACCCCCCAAGAAGGAGCACCACCACCATCACCACCACTCAGAGTCCCCAAAGGCCC
CCGTGCCACTGCTCCCACCCCTGCCCCCACCTCCACCTGAGCCCGAGAGCTCCGAGGACCCCACCAGCCCCCCTGAGC
CCCAGGACTTGAGCAGCAGCGTCTGCAAAGAGGAGAAGATGCCCAGAGGAGGCTCACTGGAGAGCGACGGCTGCCCCA
AGGAGCCAGCTAAGACTCAGCCCGCGGTTGCCACCGCCGCCACGGCCGCAGAAAAGTACAAACACCGAGGGGAGGGAG
AGCGCAAAGACATTGTTTCATCCTCCATGCCAAGGCCAAACAGAGAGGAGCCTGTGGACAGCCGGACGCCCGTGACCG
AGAGAGTTAGCTGACTTTACACGGAGCGGATTGCAAAGCAAACCAACAAGAATAAAGGCAGCTGTTGTCTCTTCTCCT
TATGGGTAGGGCTCTGACAAAGCTTCCCGATTAACTGAAATAAAAAATATTTTTTTTTCTTTCAGTAAACTTAGAGTT
TCGTGGCTTCAGGGTGGGAGTAGTTGGAGCATTGGGGATGTTTTTCTTACCGACAAGCACAGTCAGGTTGAAGACCTA
ACCAGGGCCAGAAGTAGCTTTGCACTTTTCTAAACTAGGCTCCTTCAACAAGGCTTGCTGCAGATACTACTGACCAGA
CAAGCTGTTGACCAGGCACCTCCCCTCCCGCCCAAACCTTTCCCCCATGTGGTCGTTAGAGACAGAGCGACAGAGCAG
TTGAGAGGACACTCCCGTTTTCGGTGCCATCAGTGCCCCGTCTACAGCTCCCCCAGCTCCCCCCACCTCCCCCACTCC
CAACCACGTTGGGACAGGGAGGTGTGAGGCAGGAGAGACAGTTGGATTCTTTAGAGAAGATGGATATGACCAGTGGCT
ATGGCCTGTGCGATCCCACCCGTGGTGGCTCAAGTCTGGCCCCACACCAGCCCCAATCCAAAACTGGCAAGGACGCTT
CACAGGACAGGAAAGTGGCACCTGTCTGCTCCAGCTCTGGCATGGCTAGGAGGGGGGAGTCCCTTGAACTACTGGGTG
TAGACTGGCCTGAACCACAGGAGAGGATGGCCCAGGGTGAGGTGGCATGGTCCATTCTCAAGGGACGTCCTCCAACGG
GTGGCGCTAGAGGCCATGGAGGCAGTAGGACAAGGTGCAGGCAGGCTGGCCTGGGGTCAGGCCGGGCAGAGCACAGCG
GGGTGAGAGGGATTCCTAATCACTCAGAGCAGTCTGTGACTTAGTGGACAGGGGAGGGGGCAAAGGGGGAGGAGAAGA
AAATGTTCTTCCAGTTACTTTCCAATTCTCCTTTAGGGACAGCTTAGAATTATTTGCACTATTGAGTCTTCATGTTCC
CACTTCAAAACAAACAGATGCTCTGAGAGCAAACTGGCTTGAATTGGTGACATTTAGTCCCTCAAGCCACCAGATGTG
ACAGTGTTGAGAACTACCTGGATTTGTATATATACCTGCGCTTGTTTTAAAGTGGGCTCAGCACATAGGGTTCCCACG
AAGCTCCGAAACTCTAAGTGTTTGCTGCAATTTTATAAGGACTTCCTGATTGGTTTCTCTTCTCCCCTTCCATTTCTG
CCTTTTGTTCATTTCATCCTTTCACTTCTTTCCCTTCCTCCATCCTCCTCCTTCCTAGTTCATCCCTTCTCTTCCAGG
CAGCCGCGGTGCCCAACCACACTTGTCGGCTCCAGTCCCCAGAACTCTGCCTGCCCTTTGTCCTCCTGCTGCCAGTAC
CAGCCCCACCCTGTTTTGAGCCCTGAGGAGGCCTTGGGCTCTGCTGAGTCCGACCTGGCCTGTCTGTGAAGAGCAAGA
GAGCAGCAAGGTCTTGCTCTCCTAGGTAGCCCCCTCTTCCCTGGTAAGAAAAAGCAAAAGGCATTTCCCACCCTGAAC
AACGAGCCTTTTCACCCTTCTACTCTAGAGAAGTGGACTGGAGGAGCTGGGCCCGATTTGGTAGTTGAGGAAAGCACA
GAGGCCTCCTGTGGCCTGCCAGTCATCGAGTGGCCCAACAGGGGCTCCATGCCAGCCGACCTTGACCTCACTCAGAAG
TCCAGAGTCTAGCGTAGTGCAGCAGGGCAGTAGCGGTACCAATGCAGAACTCCCAAGACCCGAGCTGGGACCAGTACC
TGGGTCCCCAGCCCTTCCTCTGCTCCCCCTTTTCCCTCGGAGTTCTTCTTGAATGGCAATGTTTTGCTTTTGCTCGAT
GCAGACAGGGGGCCAGAACACCACACATTTCACTGTCTGTCTGGTCCATAGCTGTGGTGTAGGGGCTTAGAGGCATGG
GCTTGCTGTGGGTTTTTAATTGATCAGTTTTCATGTGGGATCCCATCTTTTTAACCTCTGTTCAGGAAGTCCTTATCT
AGCTGCATATCTTCATCATATTGGTATATCCTTTTCTGTGTTTACAGAGATGTCTCTTATATCTAAATCTGTCCAACT
GAGAAGTACCTTATCAAAGTAGCAAATGAGACAGCAGTCTTATGCTTCCAGAAACACCCACAGGCATGTCCCATGTGA
GCTGCTGCCATGAACTGTCAAGTGTGTGTTGTCTTGTGTATTTCAGTTATTGTCCCTGGCTTCCTTACTATGGTGTAA
TCATGAAGGAGTGAAACATCATAGAAACTGTCTAGCACTTCCTTGCCAGTCTTTAGTGATCAGGAACCATAGTTGACA
GTTCCAATCAGTAGCTTAAGAAAAAACCGTGTTTGTCTCTTCTGGAATGGTTAGAAGTGAGGGAGTTTGCCCCGTTCT
GTTTGTAGAGTCTCATAGTTGGACTTTCTAGCATATATGTGTCCATTTCCTTATGCTGTAAAAGCAAGTCCTGCAACC
AAACTCCCATCAGCCCAATCCCTGATCCCTGATCCCTTCCACCTGCTCTGCTGATGACCCCCCCAGCTTCACTTCTGA
CTCTTCCCCAGGAAGGGAAGGGGGGTCAGAAGAGAGGGTGAGTCCTCCAGAACTCTTCCTCCAAGGACAGAAGGCTCC
TGCCCCCATAGTGGCCTCGAACTCCTGGCACTACCAAAGGACACTTATCCACGAGAGCGCAGCATCCGACCAGGTTGT
CACTGAGAAGATGTTTATTTTGGTCAGTTGGGTTTTTATGTATTATACTTAGTCAAATGTAATGTGGCTTCTGGAATC
ATTGTCCAGAGCTGCTTCCCCGTCACCTGGGCGTCATCTGGTCCTGGTAAGAGGAGTGCGTGGCCCACCAGGCCCCCC
TGTCACCCATGACAGTTCATTCAGGGCCGATGGGGCAGTCGTGGTTGGGAACACAGCATTTCAAGCGTCACTTTATTT
CATTCGGGCCCCACCTGCAGCTCCCTCAAAGAGGCAGTTGCCCAGCCTCTTTCCCTTCCAGTTTATTCCAGAGCTGCC
AGTGGGGCCTGAGGCTCCTTAGGGTTTTCTCTCTATTTCCCCCTTTCTTCCTCATTCCCTCGTCTTTCCCAAAGGCAT
CACGAGTCAGTCGCCTTTCAGCAGGCAGCCTTGGCGGTTTATCGCCCTGGCAGGCAGGGGCCCTGCAGCTCTCATGCT
GCCCCTGCCTTGGGGTCAGGTTGACAGGAGGTTGGAGGGAAAGCCTTAAGCTGCAGGATTCTCACCAGCTGTGTCCGG
CCCAGTTTTGGGGTGTGACCTCAATTTCAATTTTGTCTGTACTTGAACATTATGAAGATGGGGGCCTCTTTCAGTGAA
TTTGTGAACAGCAGAATTGACCGACAGCTTTCCAGTACCCATGGGGCTAGGTCATTAAGGCCACATCCACAGTCTCCC
CCACCCTTGTTCCAGTTGTTAGTTACTACCTCCTCTCCTGACAATACTGTATGTCGTCGAGCTCCCCCCAGGTCTACC
CCTCCCGGCCCTGCCTGCTGGTGGGCTTGTCATAGCCAGTGGGATTGCCGGTCTTGACAGCTCAGTGAGCTGGAGATA
CTTGGTCACAGCCAGGCGCTAGCACAGCTCCCTTCTGTTGATGCTGTATTCCCATATCAAAAGACACAGGGGACACCC
AGAAACGCCACATCCCCCAATCCATCAGTGCCAAACTAGCCAACGGCCCCAGCTTCTCAGCTCGCTGGATGGCGGAAG
CTGCTACTCGTGAGCGCCAGTGCGGGTGCAGACAATCTTCTGTTGGGTGGCATCATTCCAGGCCCGAAGCATGAACAG
TGCACCTGGGACAGGGAGCAGCCCCAAATTGTCACCTGCTTCTCTGCCCAGCTTTTCATTGCTGTGACAGTGATGGCG
AAAGAGGGTAATAACCAGACACAAACTGCCAAGTTGGGTGGAGAAAGGAGTTTCTTTAGCTGACAGAATCTCTGAATT
TTAAATCACTTAGTAAGCGGCTCAAGCCCAGGAGGGAGCAGAGGGATACGAGCGGAGTCCCCTGCGCGGGACCATCTG
GAATTGGTTTAGCCCAAGTGGAGCCTGACAGCCAGAACTCTGTGTCCCCCGTCTAACCACAGCTCCTTTTCCAGAGCA
TTCCAGTCAGGCTCTCTGGGCTGACTGGGCCAGGGGAGGTTACAGGTACCAGTTCTTTAAGAAGATCTTTGGGCATAT
ACATTTTTAGCCTGTGTCATTGCCCCAAATGGATTCCTGTTTCAAGTTCACACCTGCAGATTCTAGGACCTGTGTCCT
AGACTTCAGGGAGTCAGCTGTTTCTAGAGTTCCTACCATGGAGTGGGTCTGGAGGACCTGCCCGGTGGGGGGGCAGAG
CCCTGCTCCCTCCGGGTCTTCCTACTCTTCTCTCTGCTCTGACGGGATTTGTTGATTCTCTCCATTTTGGTGTCTTTC
TCTTTTAGATATTGTATCAATCTTTAGAAAAGGCATAGTCTACTTGTTATAAATCGTTAGGATACTGCCTCCCCCAGG
GTCTAAAATTACATATTAGAGGGGAAAAGCTGAACACTGAAGTCAGTTCTCAACAATTTAGAAGGAAAACCTAGAAAA
CATTTGGCAGAAAATTACATTTCGATGTTTTTGAATGAATACGAGCAAGCTTTTACAACAGTGCTGATCTAAAAATAC
TTAGCACTTGGCCTGAGATGCCTGGTGAGCATTACAGGCAAGGGGAATCTGGAGGTAGCCGACCTGAGGACATGGCTT
CTGAACCTGTCTTTTGGGAGTGGTATGGAAGGTGGAGCGTTCACCAGTGACCTGGAAGGCCCAGCACCACCCTCCTTC
CCACTCTTCTCATCTTGACAGAGCCTGCCCCAGCGCTGACGTGTCAGGAAAACACCCAGGGAACTAGGAAGGCACTTC
TGCCTGAGGGGCAGCCTGCCTTGCCCACTCCTGCTCTGCTCGCCTCGGATCAGCTGAGCCTTCTGAGCTGGCCTCTCA
CTGCCTCCCCAAGGCCCCCTGCCTGCCCTGTCAGGAGGCAGAAGGAAGCAGGTGTGAGGGCAGTGCAAGGAGGGAGCA
CAACCCCCAGCTCCCGCTCCGGGCTCCGACTTGTGCACAGGCAGAGCCCAGACCCTGGAGGAAATCCTACCTTTGAAT
TCAAGAACATTTGGGGAATTTGGAAATCTCTTTGCCCCCAAACCCCCATTCTGTCCTACCTTTAATCAGGTCCTGCTC
AGCAGTGAGAGCAGATGAGGTGAAAAGGCCAAGAGGTTTGGCTCCTGCCCACTGATAGCCCCTCTCCCCGCAGTGTTT
GTGTGTCAAGTGGCAAAGCTGTTCTTCCTGGTGACCCTGATTATATCCAGTAACACATAGACTGTGCGCATAGGCCTG
CTTTGTCTCCTCTATCCTGGGCTTTTGTTTTGCTTTTTAGTTTTGCTTTTAGTTTTTCTGTCCCTTTTATTTAACGCA
CCGACTAGACACACAAAGCAGTTGAATTTTTATATATATATCTGTATATTGCACAATTATAAACTCATTTTGCTTGTG
GCTCCACACACACAAAAAAAGACCTGTTAAAATTATACCTGTTGCTTAATTACAATATTTCTGATAACCATAGCATAG
GACAAGGGAAAATAAAAAAAGAAAAAAAAGAAAAAAAAACGACAAATCTGTCTGCTGGTCACTTCTTCTGTCCAAGCA
GATTCGTGGTCTTTTCCTCGCTTCTTTCAAGGGCTTTCCTGTGCCAGGTGAAGGAGGCTCCAGGCAGCACCCAGGTTT
TGCACTCTTGTTTCTCCCGTGCTTGTGAAAGAGGTCCCAAGGTTCTGGGTGCAGGAGCGCTCCCTTGACCTGCTGAAG
TCCGGAACGTAGTCGGCACAGCCTGGTCGCCTTCCACCTCTGGGAGCTGGAGTCCACTGGGGTGGCCTGACTCCCCCA
GTCCCCTTCCCGTGACCTGGTCAGGGTGAGCCCATGTGGAGTCAGCCTCGCAGGCCTCCCTGCCAGTAGGGTCCGAGT
GTGTTTCATCCTTCCCACTCTGTCGAGCCTGGGGGCTGGAGCGGAGACGGGAGGCCTGGCCTGTCTCGGAACCTGTGA
GCTGCACCAGGTAGAACGCCAGGGACCCCAGAATCATGTGCGTCAGTCCAAGGGGTCCCCTCCAGGAGTAGTGAAGAC
TCCAGAAATGTCCCTTTCTTCTCCCCCATCCTACGAGTAATTGCATTTGCTTTTGTAATTCTTAATGAGCAATATCTG
CTAGAGAGTTTAGCTGTAACAGTTCTTTTTGATCATCTTTTTTTAATAATTAGAAACACCAAAAAAATCCAGAAACTT
GTTCTTCCAAAGCAGAGAGCATTATAATCACCAGGGCCAAAAGCTTCCCTCCCTGCTGTCATTGCTTCTTCTGAGGCC
TGAATCCAAAAGAAAAACAGCCATAGGCCCTTTCAGTGGCCGGGCTACCCGTGAGCCCTTCGGAGGACCAGGGCTGGG
GCAGCCTCTGGGCCCACATCCGGGGCCAGCTCCGGCGTGTGTTCAGTGTTAGCAGTGGGTCATGATGCTCTTTCCCAC
CCAGCCTGGGATAGGGGCAGAGGAGGCGAGGAGGCCGTTGCCGCTGATGTTTGGCCGTGAACAGGTGGGTGTCTGCGT
GCGTCCACGTGCGTGTTTTCTGACTGACATGAAATCGACGCCCGAGTTAGCCTCACCCGGTGACCTCTAGCCCTGCCC
GGATGGAGCGGGGCCCACCCGGTTCAGTGTTTCTGGGGAGCTGGACAGTGGAGTGCAAAAGGCTTGCAGAACTTGAAG
CCTGCTCCTTCCCTTGCTACCACGGCCTCCTTTCCGTTTGATTTGTCACTGCTTCAATCAATAACAGCCGCTCCAGAG
TCAGTAGTCAATGAATATATGACCAAATATCACCAGGACTGTTACTCAATGTGTGCCGAGCCCTTGCCCATGCTGGGC
TCCCGTGTATCTGGACACTGTAACGTGTGCTGTGTTTGCTCCCCTTCCCCTTCCTTCTTTGCCCTTTACTTGTCTTTC
TGGGGTTTTTCTGTTTGGGTTTGGTTTGGTTTTTATTTCTCCTTTTGTGTTCCAAACATGAGGTTCTCTCTACTGGTC
CTCTTAACTGTGGTGTTGAGGCTTATATTTGTGTAATTTTTGGTGGGTGAAAGGAATTTTGCTAAGTAAATCTCTTCT
GTGTTTGAACTGAAGTCTGTATTGTAACTATGTTTAAAGTAATTGTTCCAGAGACAAATATTTCTAGACACTTTTTCT
TTACAAACAAAAGCATTCGGAGGGAGGGGGATGGTGACTGAGATGAGAGGGGAGAGCTGAACAGATGACCCCTGCCCA
GATCAGCCAGAAGCCACCCAAAGCAGTGGAGCCCAGGAGTCCCACTCCAAGCCAGCAAGCCGAATAGCTGATGTGTTG
CCACTTTCCAAGTCACTGCAAAACCAGGTTTTGTTCCGCCCAGTGGATTCTTGTTTTGCTTCCCCTCCCCCCGAGATT
ATTACCACCATCCCGTGCTTTTAAGGAAAGGCAAGATTGATGTTTCCTTGAGGGGAGCCAGGAGGGGATGTGTGTGTG
CAGAGCTGAAGAGCTGGGGAGAATGGGGCTGGGCCCACCCAAGCAGGAGGCTGGGACGCTCTGCTGTGGGCACAGGTC
AGGCTAATGTTGGCAGATGCAGCTCTTCCTGGACAGGCCAGGTGGTGGGCATTCTCTCTCCAAGGTGTGCCCCGTGGG
CATTACTGTTTAAGACACTTCCGTCACATCCCACCCCATCCTCCAGGGCTCAACACTGTGACATCTCTATTCCCCACC
CTCCCCTTCCCAGGGCAATAAAATGACCATGGAGGGGGCTTGCACTCTCTTGGCTGTCACCCGATCGCCAGCAAAACT
TAGATGTGAGAAAACCCCTTCCCATTCCATGGCGAAAACATCTCCTTAGAAAAGCCATTACCCTCATTAGGCATGGTT
TTGGGCTCCCAAAACACCTGACAGCCCCTCCCTCCTCTGAGAGGCGGAGAGTGCTGACTGTAGTGACCATTGCATGCC
GGGTGCAGCATCTGGAAGAGCTAGGCAGGGTGTCTGCCCCCTCCTGAGTTGAAGTCATGCTCCCCTGTGCCAGCCCAG
AGGCCGAGAGCTATGGACAGCATTGCCAGTAACACAGGCCACCCTGTGCAGAAGGGAGCTGGCTCCAGCCTGGAAACC
TGTCTGAGGTTGGGAGAGGTGCACTTGGGGCACAGGGAGAGGCCGGGACACACTTAGCTGGAGATGTCTCTAAAAGCC
CTGTATCGTATTCACCTTCAGTTTTTGTGTTTTGGGACAATTACTTTAGAAAATAAGTAGGTCGTTTTAAAAACAAAA
ATTATTGATTGCTTTTTTGTAGTGTTCAGAAAAAAGGTTCTTTGTGTATAGCCAAATGACTGAAAGCACTGATATATT
TAAAAACAAAAGGCAATTTATTAAGGAAATTTGTACCATTTCAGTAAACCTGTCTGAATGTACCTGTATACGTTTCAA
AAACACCCCCCCCCCACTGAATCCCTGTAACCTATTTATTATATAAAGAGTTTGCCTTATAAATTTACATAAAAATGT
C
SEQ ID NO: 37 >NP_001104262.1 MECP2 [organism = Homo sapiens] [GeneID = 4204]
[isoform = 2]
MAAAAAAAPSGGGGGGEEERLEEKSEDQDLQGLKDKPLKFKKVKKDKKEEKEGKHEPVOPSAHHSAEPAEAGKAETSE
GSGSAPAVPEASASPKQRRSIIRDRGPMYDDPTLPEGWTRKLKQRKSGRSAGKYDVYLINPQGKAFRSKVELIAYFEK
VGDTSLDPNDFDFTVTGRGSPSRREQKPPKKPKSPKAPGTGRGRGRPKGSGTTRPKAATSEGVQVKRVLEKSPGKLLV
KMPFQTSPGGKAEGGGATTSTQVMVIKRPGRKRKAEADPQAIPKKRGRKPGSVVAAAAAEAKKKAVKESSIRSVQETV
LPIKKRKTRETVSIEVKEVVKPLLVSTLGEKSGKGLKTCKSPGRKSKESSPKGRSSSASSPPKKEHHHHHHHSESPKA
PVPLLPPLPPPPPEPESSEDPTSPPEPQDLSSSVCKEEKMPRGGSLESDGCPKEPAKTQPAVATAATAAEKYKHRGEG
ERKDIVSSSMPRPNREEPVDSRTPVTERVS
SEQ ID NO: 38 >NP_001303266.1 MECP2 [organism = Homo sapiens] [GeneID = 4204]
[isoform = 3]
MYDDPTLPEGWTRKLKQRKSGRSAGKYDVYLINPQGKAFRSKVELIAYFEKVGDTSLDPNDFDFTVTGRGSPSRREQK
PPKKPKSPKAPGTGRGRGRPKGSGTTRPKAATSEGVQVKRVLEKSPGKLLVKMPFQTSPGGKAEGGGATTSTQVMVIK
RPGRKRKAEADPQAIPKKRGRKPGSVVAAAAAEAKKKAVKESSIRSVQETVLPIKKRKTRETVSIEVKEVVKPLLVST
LGEKSGKGLKTCKSPGRKSKESSPKGRSSSASSPPKKEHHHHHHHSESPKAPVPLLPPLPPPPPEPESSEDPTSPPEP
QDLSSSVCKEEKMPRGGSLESDGCPKEPAKTQPAVATAATAAEKYKHRGEGERKDIVSSSMPRPNREEPVDSRTPVTE
SEQ ID NO: 39 >NP_001373066.1 MECP2 [organism-Homo sapiens] [GeneID = 4204]
[isoform = 4]
MPFQTSPGGKAEGGGATTSTQVMVIKRPGRKRKAEADPQAIPKKRGRKPGSVVAAAAAEAKKKAVKESSIRSVQETVL
PIKKRKTRETVSIEVKEVVKPLLVSTLGEKSGKGLKTCKSPGRKSKESSPKGRSSSASSPPKKEHHHHHHHSESPKAP
VPLLPPLPPPPPEPESSEDPTSPPEPQDLSSSVCKEEKMPRGGSLESDGCPKEPAKTQPAVATAATAAEKYKHRGEGE
RKDIVSSSMPRPNREEPVDSRTPVTERVS
SEQ ID NO: 40 >NP_004983.1 MECP2 [organism = Homo sapiens] [GeneID = 4204]
[isoform = 1]
MVAGMLGLREEKSEDQDLQGLKDKPLKFKKVKKDKKEEKEGKHEPVQPSAHHSAEPAEAGKAETSEGSGSAPAVPEAS
ASPKQRRSIIRDRGPMYDDPTLPEGWTRKLKQRKSGRSAGKYDVYLINPQGKAFRSKVELIAYFEKVGDTSLDPNDED
FTVTGRGSPSRREQKPPKKPKSPKAPGTGRGRGRPKGSGTTRPKAATSEGVQVKRVLEKSPGKLLVKMPFQTSPGGKA
EGGGATTSTQVMVIKRPGRKRKAEADPQAIPKKRGRKPGSVVAAAAAEAKKKAVKESSIRSVQETVLPIKKRKTRETV
SIEVKEVVKPLLVSTLGEKSGKGLKTCKSPGRKSKESSPKGRSSSASSPPKKEHHHHHHHSESPKAPVPLLPPLPPPP
PEPESSEDPTSPPEPQDLSSSVCKEEKMPRGGSLESDGCPKEPAKTQPAVATAATAAEKYKHRGEGERKDIVSSSMPR
PNREEPVDSRTPVTERVS
SEQ ID NO: 41 >XP_011529468.1 MECP2 [organism = Homo sapiens] [GeneID = 4204]
[isoform = X1]
MYDDPTLPEGWTRKLKQRKSGRSAGKYDVYLINPQGKAFRSKVELIAYFEKVGDTSLDPNDFDFTVTGRGSPSRREQK
PPKKPKSPKAPGTGRGRGRPKGSGTTRPKAATSEGVQVKRVLEKSPGKLLVKMPFQTSPGGKAEGGGATTSTQVMVIK
RPGRKRKAEADPQAIPKKRGRKPGSVVAAAAAEAKKKAVKESSIRSVQETVLPIKKRKTRETVSIEVKEVVKPLLVST
LGEKSGKGLKTCKSPGRKSKESSPKGRSSSASSPPKKEHHHHHHHSESPKAPVPLLPPLPPPPPEPESSEDPTSPPEP
QDLSSSVCKEEKMPRGGSLESDGCPKEPAKTQPAVATAATAAEKYKHRGEGERKDIVSSSMPRPNREEPVDSRTPVTE
RVS
SEQ ID NO: 42 >sp|P11940|PABP1_HUMAN Polyadenylate-binding protein 1 OS = Homo
sapiens OX = 9606 GN = PABPCI PE = 1 SV = 2
MNPSAPSYPMASLYVGDLHPDVTEAMLYEKFSPAGPILSIRVCRDMITRRSLGYAYVNFQQPADAERALDTMNEDVIK
GKPVRIMWSQRDPSLRKSGVGNIFIKNLDKSIDNKALYDTFSAFGNILSCKVVCDENGSKGYGFVHFETQEAAERAIE
KMNGMLLNDRKVFVGRFKSRKEREAELGARAKEFTNVYIKNFGEDMDDERLKDLFGKFGPALSVKVMTDESGKSKGFG
FVSFERHEDAQKAVDEMNGKELNGKQIYVGRAQKKVERQTELKRKFEQMKQDRITRYQGVNLYVKNLDDGIDDERLRK
EFSPFGTITSAKVMMEGGRSKGFGFVCFSSPEEATKAVTEMNGRIVATKPLYVALAQRKEERQAHLTNQYMQRMASVR
AVPNPVINPYQPAPPSGYFMAAIPQTQNRAAYYPPSQIAQLRPSPRWTAQGARPHPFQNMPGAIRPAAPRPPFSTMRP
ASSQVPRVMSTQRVANTSTQTMGPRPAAAAAAATPAVRTVPQYKYAAGVRNPQQHLNAQPQVTMQQPAVHVQGQEPLT
ASMLASAPPQEQKQMLGERLFPLIQAMHPTLAGKITGMLLEIDNSELLHMLESPESLRSKVDEAVAVLQAHQAKEAAQ
KAVNSATGVPTV
SEQ ID NO: 43 >sp|P11940-2|PABP1_HUMAN Isoform 2 of Polyadenylate-binding protein 1
OS = Homo sapiens OX = 9606 GN = PABPC1
MNPSAPSYPMASLYVGDLHPDVTEAMLYEKFSPAGPILSIRVCRDMITRRSLGYAYVNFQQPADAERALDTMNEDVIK
GKPVRIMWSQRDPSLRKSGVGNIFIKNLDKSIDNKALYDTFSAFGNILSCKVVCDENGSKGYGFVHFETQEAAERAIE
KMNGMLLNDRKVFVGRFKSRKEREAELGARAKEFTNVYIKNFGEDMDDERLKDLFGKFGPALSVKVMTDESGKSKGFG
FVSFERHEDAQKAVDEMNGKELNGKQIYVGRAQKKVERQTELKRKFEQMKQDRITRYQGVNLYVKNLDDGIDDERLRK
EFSPFGTITSAKVMMEGGRSKGFGFVCFSSPEEATKAVTEMNGRIVATKPLYVALAQRKEERQAHLTNQYMQRMASVR
AVPNPVINPYQPAPPSGYFMAAIPQTQNRAAYYPPSQIAQLRPSPRWTAQGARPHPAVHVQGQEPLTASMLASAPPQE
QKQMLGERLFPLIQAMHPTLAGKITGMLLEIDNSELLHMLESPESLRSKVDEAVAVLQAHQAKEAAQKAVNSATGVPT
V
SEQ ID NO: 44 >sp|Q9H0A0|NAT10_HUMAN RNA cytidine acetyltransferase OS = Homo
sapiens OX = 9606 GN = NAT10 PE-1 SV = 2
MHRKKVDNRIRILIENGVAERQRSLFVVVGDRGKDQVVILHHMLSKATVKARPSVLWCYKKELGFSSHRKKRMRQLQK
KIKNGTLNIKQDDPFELFIAATNIRYCYYNETHKILGNTFGMCVLQDFEALTPNLLARTVETVEGGGLVVILLRTMNS
LKQLYTVTMDVHSRYRTEAHQDVVGRENERFILSLASCKKCLVIDDQLNILPISSHVATMEALPPQTPDESLGPSDLE
LRELKESLQDTQPVGVLVDCCKTLDQAKAVLKFIEGISEKTLRSTVALTAARGRGKSAALGLAIAGAVAFGYSNIFVT
SPSPDNLHTLFEFVFKGFDALQYQEHLDYEIIQSLNPEFNKAVIRVNVFREHRQTIQYIHPADAVKLGQAELVVIDEA
AAIPLPLVKSLLGPYLVFMASTINGYEGTGRSLSLKLIQQLRQQSAQSQVSTTAENKTTTTARLASARTLYEVSLQES
IRYAPGDAVEKWLNDLLCLDCLNITRIVSGCPLPEACELYYVNRDTLFCYHKASEVFLQRLMALYVASHYKNSPNDLQ
MLSDAPAHHLFCLLPPVPPTQNALPEVLAVIQVCLEGEISRQSILNSLSRGKKASGDLIPWTVSEQFQDPDFGGLSGG
RVVRIAVHPDYQGMGYGSRALQLLQMYYEGRFPCLEEKVLETPQEIHTVSSEAVSLLEEVITPRKDLPPLLLKLNERP
AERLDYLGVSYGLTPRLLKFWKRAGFVPVYLRQTPNDLTGEHSCIMLKTLTDEDEADQGGWLAAFWKDFRRRFLALLS
YQFSTFSPSLALNIIQNRNMGKPAQPALSREELEALFLPYDLKRLEMYSRNMVDYHLIMDMIPAISRIYFLNQLGDLA
LSAAQSALLLGIGLQHKSVDQLEKEIELPSGQLMGLENRIIRKVVKLFNEVQEKAIEEQMVAAKDVVMEPTMKTLSDD
LDEAAKEFQEKHKKEVGKLKSMDLSEYIIRGDDEEWNEVLNKAGPNASIISLKSDKKRKLEAKQEPKQSKKLKNRETK
NKKDMKLKRKK
SEQ ID NO: 45 >sp|Q9H0A0-2|NAT10_HUMAN Isoform 2 of RNA cytidine acetyltransferase
OS = Homo sapiens OX = 9606 GN = NAT10
MRQLQKKIKNGTLNIKQDDPFELFIAATNIRYCYYNETHKILGNTFGMCVLQDFEALTPNLLARTVETVEGGGLVVIL
LRTMNSLKQLYTVTMDVHSRYRTEAHQDVVGRENERFILSLASCKKCLVIDDQLNILPISSHVATMEALPPQTPDESL
GPSDLELRELKESLQDTQPVGVLVDCCKTLDQAKAVLKFIEGISEKTLRSTVALTAARGRGKSAALGLAIAGAVAFGY
SNIFVTSPSPDNLHTLFEFVFKGEDALQYQEHLDYEIIQSLNPEFNKAVIRVNVFREHRQTIQYIHPADAVKLGQAEL
VVIDEAAAIPLPLVKSLLGPYLVEMASTINGYEGTGRSLSLKLIQQLRQQSAQSQVSTTAENKTTTTARLASARTLYE
VSLQESIRYAPGDAVEKWINDLLCLDCLNITRIVSGCPLPEACELYYVNRDTLFCYHKASEVFLQRLMALYVASHYKN
SPNDLQMLSDAPAHHLFCLLPPVPPTQNALPEVLAVIQVCLEGEISRQSILNSLSRGKKASGDLIPWTVSEQFQDPDF
GGLSGGRVVRIAVHPDYQGMGYGSRALQLLQMYYEGRFPCLEEKVLETPQEIHTVSSEAVSLLEEVITPRKDLPPLLL
KLNERPAERLDYLGVSYGLTPRLLKFWKRAGFVPVYLRQTPNDLTGEHSCIMLKTLTDEDEADQGGWLAAFWKDERRR
FLALLSYQFSTESPSLALNIIQNRNMGKPAQPALSREELEALFLPYDLKRLEMYSRNMVDYHLIMDMIPAISRIYFLN
QLGDLALSAAQSALLLGIGLQHKSVDQLEKEIELPSGQLMGLENRIIRKVVKLFNEVQEKAIEEQMVAAKDVVMEPTM
KTLSDDLDEAAKEFQEKHKKEVGKLKSMDLSEYIIRGDDEEWNEVLNKAGPNASIISLKSDKKRKLEAKQEPKQSKKL
KNRETKNKKDMKLKRKK
SEQ ID NO: 46 spCas9
MDKKYSIGLDIGTNSVGWAVITDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFRIPYYVGPLARGNSRFAWMTRK
SEETITPWNFEEVVDKGASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRENASLGTYHDLLKIIKDKDELDNEENEDILEDIVLTLT
LFEDREMIEERLKTYAHLEDDKVMKQLKRRRYTGWGRLSRKLINGIRDKQSGKTILDELKSDGFANRNFMQLIHDDSL
TFKEDIQKAQVSGQGDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDHIVPQSFLKDDSIDNKVLT
RSDKNRGKSDNVPSEEVVKKMKNYWRQLLNAKLITQRKEDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGD
SEQ ID NO: 47 dCas13
MNIPALVENQKKYFGTYSVMAMLNAQTVLDHIQKVADIEGEQNENNENLWFHPVMSHLYNAKNGYDKQPEKTMFIIER
LQSYFPFLKIMAENQREYSNGKYKQNRVEVNSNDIFEVLKRAFGVLKMYRDLTNAYKTYEEKLNDGCEFLTSTEQPLS
GMINNYYTVALRNMNERYGYKTEDLAFIQDKRFKFVKDAYGKKKSQVNTGFFLSLQDYNGDTQKKLHLSGVGIALLIC
LFLDKQYINIFLSRLPIFSSYNAQSEERRIIIRSFGINSIKLPKDRIHSEKSNKSVAMDMLNEVKRCPDELFTTLSAE
KQSRFRIISDDHNEVLMKRSSDRFVPLLLQYIDYGKLFDHIRFHVNMGKLRYLLKADKTCIDGQTRVRVIEQPLNGFG
RLEEAETMRKQENGTFGNSGIRIRDFENMKRDDANPANYPYIVDTYTHYILENNKVEMFINDKEDSAPLLPVIEDDRY
VVKTIPSCRMSTLEIPAMAFHMFLFGSKKTEKLIVDVHNRYKRLFQAMQKEEVTAENIASFGIAESDLPQKILDLISG
NAHGKDVDAFIRLTVDDMLTDTERRIKRFKDDRKSIRSADNKMGKRGFKQISTGKLADFLAKDIVLFQPSVNDGENKI
TGLNYRIMQSAIAVYDSGDDYEAKQQFKLMFEKARLIGKGTTEPHPFLYKVFARSIPANAVEFYERYLIERKFYLTGL
SNEIKKGNRVDVPFIRRDQNKWKTPAMKTLGRIYSEDLPVELPRQMEDNEIKSHLKSLPQMEGIDENNANVTYLIAEY
MKRVLDDDFQTFYQWNRNYRYMDMLKGEYDRKGSLQHCFTSVEEREGLWKERASRTERYRKQASNKIRSNRQMRNASS
EEIETILDKRLSNSRNEYQKSEKVIRRYRVQDALLFLLAKKTLTELADEDGEREKLKEIMPDAEKGILSEIMPMSFTF
EKGGKKYTITSEGMKLKNYGDFFVLASDKRIGNLLELVGSDIVSKEDIMEEFNKYDQCRPEISSIVENLEKWAFDTYP
ELSARVDREEKVDEKSILKILLNNKNINKEQSDILRKIRNAFDANNYPDKGVVEIKALPEIAMSIKKAFGEYAIMKGS
LQ
SEQ ID NO: 48 PABC1-dCas13 Fusion Protein
MNIPALVENQKKYFGTYSVMAMLNAQTVLDHIQKVADIEGEQNENNENLWFHPVMSHLYNAKNGYDKQPEKTMFIIER
LQSYFPFLKIMAENQREYSNGKYKQNRVEVNSNDIFEVLKRAFGVLKMYRDLTNAYKTYEEKLNDGCEFLTSTEQPLS
GMINNYYTVALRNMNERYGYKTEDLAFIQDKRFKFVKDAYGKKKSQVNTGFFLSLQDYNGDTQKKLHLSGVGIALLIC
LFLDKQYINIFLSRLPIFSSYNAQSEERRIIIRSFGINSIKLPKDRIHSEKSNKSVAMDMLNEVKRCPDELFTTLSAE
KQSRFRIISDDHNEVLMKRSSDRFVPLLLQYIDYGKLFDHIRFHVNMGKLRYLLKADKTCIDGQTRVRVIEQPLNGFG
RLEEAETMRKQENGTFGNSGIRIRDFENMKRDDANPANYPYIVDTYTHYILENNKVEMFINDKEDSAPLLPVIEDDRY
VVKTIPSCRMSTLEIPAMAFHMFLFGSKKTEKLIVDVHNRYKRLFQAMQKEEVTAENIASFGIAESDLPQKILDLISG
NAHGKDVDAFIRLTVDDMLTDTERRIKRFKDDRKSIRSADNKMGKRGFKQISTGKLADFLAKDIVLFQPSVNDGENKI
TGLNYRIMQSAIAVYDSGDDYEAKQQFKLMFEKARLIGKGTTEPHPFLYKVFARSIPANAVEFYERYLIERKFYLTGL
SNEIKKGNRVDVPFIRRDQNKWKTPAMKTLGRIYSEDLPVELPRQMEDNEIKSHLKSLPQMEGIDENNANVTYLIAEY
MKRVLDDDFQTFYQWNRNYRYMDMLKGEYDRKGSLQHCFTSVEEREGLWKERASRTERYRKQASNKIRSNRQMRNASS
EEIETILDKRLSNSRNEYQKSEKVIRRYRVQDALLFLLAKKTLTELADEDGEREKLKEIMPDAEKGILSEIMPMSFTF
EKGGKKYTITSEGMKLKNYGDFFVLASDKRIGNLLELVGSDIVSKEDIMEEFNKYDQCRPEISSIVENLEKWAFDTYP
ELSARVDREEKVDFKSILKILLNNKNINKEQSDILRKIRNAFDANNYPDKGVVEIKALPEIAMSIKKAFGEYAIMKGS
LQMNPSAPSYPMASLYVGDLHPDVTEAMLYEKFSPAGPILSIRVCRDMITRRSLGYAYVNFQQPADAERALDTMNFDV
IKGKPVRIMWSQRDPSLRKSGVGNIFIKNLDKSIDNKALYDTFSAFGNILSCKVVCDENGSKGYGFVHFETQEAAERA
IEKMNGMLLNDRKVFVGRFKSRKEREAELGARAKEFTNVYIKNFGEDMDDERLKDLFGKFGPALSVKVMTDESGKSKG
FGFVSFERHEDAQKAVDEMNGKELNGKQIYVGRAQKKVERQTELKRKFEQMKQDRITRYQGVNLYVKNLDDGIDDERL
RKEFSPFGTITSAKVMMEGGRSKGFGFVCFSSPEEATKAVTEMNGRIVATKPLYVALAQRKEERQAHLTNQYMQRMAS
VRAVPNPVINPYQPAPPSGYEMAAIPQTQNRAAYYPPSQIAQLRPSPRWTAQGARPHPFQNMPGAIRPAAPRPPFSTM
RPASSQVPRVMSTQRVANTSTQTMGPRPAAAAAAATPAVRTVPQYKYAAGVRNPQQHLNAQPQVTMQQPAVHVQGQEP
LTASMLASAPPQEQKQMLGERLFPLIQAMHPTLAGKITGMLLEIDNSELLHMLESPESLRSKVDEAVAVLQAHQAKEA
AQKAVNSATGVPTV
SEQ ID NO: 49 PABC1-dCas13 Fusion Protein
MNIPALVENQKKYFGTYSVMAMLNAQTVLDHIQKVADIEGEQNENNENLWFHPVMSHLYNAKNGYDKQPEKTMFIIER
LQSYFPFLKIMAENQREYSNGKYKQNRVEVNSNDIFEVLKRAFGVLKMYRDLTNAYKTYEEKLNDGCEFLTSTEQPLS
GMINNYYTVALRNMNERYGYKTEDLAFIQDKRFKFVKDAYGKKKSQVNTGFFLSLQDYNGDTQKKLHLSGVGIALLIC
LFLDKQYINIFLSRLPIESSYNAQSEERRIIIRSFGINSIKLPKDRIHSEKSNKSVAMDMLNEVKRCPDELFTTLSAE
KQSRFRIISDDHNEVLMKRSSDRFVPLLLQYIDYGKLFDHIRFHVNMGKLRYLLKADKTCIDGQTRVRVIEQPLNGFG
RLEEAETMRKQENGTFGNSGIRIRDFENMKRDDANPANYPYIVDTYTHYILENNKVEMFINDKEDSAPLLPVIEDDRY
VVKTIPSCRMSTLEIPAMAFHMFLFGSKKTEKLIVDVHNRYKRLFQAMQKEEVTAENIASFGIAESDLPQKILDLISG
NAHGKDVDAFIRLTVDDMLTDTERRIKRFKDDRKSIRSADNKMGKRGFKQISTGKLADFLAKDIVLFQPSVNDGENKI
TGLNYRIMQSAIAVYDSGDDYEAKQQFKLMFEKARLIGKGTTEPHPFLYKVFARSIPANAVEFYERYLIERKFYLTGL
SNEIKKGNRVDVPFIRRDQNKWKTPAMKTLGRIYSEDLPVELPRQMEDNEIKSHLKSLPQMEGIDENNANVTYLIAEY
MKRVLDDDFQTFYQWNRNYRYMDMLKGEYDRKGSLQHCFTSVEEREGLWKERASRTERYRKQASNKIRSNRQMRNASS
EEIETILDKRLSNSRNEYQKSEKVIRRYRVQDALLFLLAKKTLTELADFDGEREKLKEIMPDAEKGILSEIMPMSFTF
EKGGKKYTITSEGMKLKNYGDFFVLASDKRIGNLLELVGSDIVSKEDIMEEFNKYDQCRPEISSIVENLEKWAFDTYP
ELSARVDREEKVDFKSILKILLNNKNINKEQSDILRKIRNAFDANNYPDKGVVEIKALPEIAMSIKKAFGEYAIMKGS
LQLENPSAPSYPMASLYVGDLHPDVTEAMLYEKFSPAGPILSIRVCRDMITRRSLGYAYVNFQQPADAERALDTMNED
VIKGKPVRIMWSQRDPSLRKSGVGNIFIKNLDKSIDNKALYDTFSAFGNILSCKVVCDENGSKGYGFVHFETQEAAER
AIEKMNGMLLNDRKVFVGRFKSRKEREAELGARAKEFTNVYIKNFGEDMDDERLKDLFGKFGPALSVKVMTDESGKSK
GFGFVSFERHEDAQKAVDEMNGKELNGKQIYVGRAQKKVERQTELKRKFEQMKQDRITRYQGVNLYVKNLDDGIDDER
LRKEFSPFGTITSAKVMMEGGRSKGFGFVCESSPEEATKAVTEMNGRIVATKPLYVALAQRKEERQAHLTNQYMQRMA
SVRAVPNPVINPYQPAPPSGYFMAAIPQTQNRAAYYPPSQIAQLRPSPRWTAQGARPHPFQNMPGAIRPAAPRPPEST
MRPASSQVPRVMSTQRVANTSTQTMGPRPAAAAAAATPAVRTVPQYKYAAGVRNPQQHLNAQPQVTMQQPAVHVQGQE
PLTASMLASAPPQEQKQMLGERLFPLIQAMHPTLAGKITGMLLEIDNSELLHMLESPESLRSKVDEAVAVLQAHQAKE
AAQKAVNSATGVPTV*
SEQ ID NO: 50 >pJC1211 (dCAS13-PABP) (10,647 bp)
CTTTAATAGTGGACTCTTGTTCCAAACTGGAACAACACTCAACCCTATCTCGGTCTATTCTTTTGATTTATAAGGGAT
TTTGCCGATTTCGGCCTATTGGTTAAAAAATGAGCTGATTTAACAAAAATTTAACGCGAATTAATTCTGTGGAATGTG
TGTCAGTTAGGGTGTGGAAAGTCCCCAGGCTCCCCAGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCTCCGCCCCATGGCTGACTAATT
TTTTTTATTTATGCAGAGGCCGAGGCCGCCTCTGCCTCTGAGCTATTCCAGAAGTAGTGAGGAGGCTTTTTTGGAGGC
CTAGGCTTTTGCAAAAAGCTCCCGGGAGCTTGTATATCCATTTTCGGATCTGATCAAGAGACAGGATGAGGATCGTTT
CGCATGATTGAACAAGATGGATTGCACGCAGGTTCTCCGGCCGCTTGGGTGGAGAGGCTATTCGGCTATGACTGGGCA
CAACAGACAATCGGCTGCTCTGATGCCGCCGTGTTCCGGCTGTCAGCGCAGGGGCGCCCGGTTCTTTTTGTCAAGACC
GACCTGTCCGGTGCCCTGAATGAACTGCAGGACGAGGCAGCGCGGCTATCGTGGCTGGCCACGACGGGCGTTCCTTGC
GCAGCTGTGCTCGACGTTGTCACTGAAGCGGGAAGGGACTGGCTGCTATTGGGCGAAGTGCCGGGGCAGGATCTCCTG
TCATCTCACCTTGCTCCTGCCGAGAAAGTATCCATCATGGCTGATGCAATGCGGCGGCTGCATACGCTTGATCCGGCT
ACCTGCCCATTCGACCACCAAGCGAAACATCGCATCGAGCGAGCACGTACTCGGATGGAAGCCGGTCTTGTCGATCAG
GATGATCTGGACGAAGAGCATCAGGGGCTCGCGCCAGCCGAACTGTTCGCCAGGCTCAAGGCGCGCATGCCCGACGGC
GAGGATCTCGTCGTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCTGAAGAGCTTGGC
GGCGAATGGGCTGACCGCTTCCTCGTGCTTTACGGTATCGCCGCTCCCGATTCGCAGCGCATCGCCTTCTATCGCCTT
CTTGACGAGTTCTTCTGAGCGGGACTCTGGGGTTCGAAATGACCGACCAAGCGACGCCCAACCTGCCATCACGAGATT
TCGATTCCACCGCCGCCTTCTATGAAAGGTTGGGCTTCGGAATCGTTTTCCGGGACGCCGGCTGGATGATCCTCCAGC
GCGGGGATCTCATGCTGGAGTTCTTCGCCCACCCCAACTTGTTTATTGCAGCTTATAATGGTTACAAATAAAGCAATA
GCATCACAAATTTCACAAATAAAGCATTTTTTTCACTGCATTCTAGTTGTGGTTTGTCCAAACTCATCAATGTATCTT
ATCATGTCTGTATACCGTCGACCTCTAGCTAGAGCTTGGCGTAATCATGGTCATAGCTGTTTCCTGTGTGAAATTGTT
ATCCGCTCACAATTCCACACAACATACGAGCCGGAAGCATAAAGTGTAAAGCCTGGGGTGCCTAATGAGTGAGCTAAC
TCACATTAATTGCGTTGCGCTCACTGCCCGCTTTCCAGTCGGGAAACCTGTCGTGCCAGCTGCATTAATGAATCGGCC
AACGCGCGGGGAGAGGCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGGATAACGCAGGAAAGA
ACATGTGAGCAAAAGGCCAGCAAAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTTTTGTTTGCAAGCAGCAGA
TTACGCGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAGTTTTA
AATCAATCTAAAGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCATTCTGAGAATAGTGTA
TGCGGCGACCGAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCATACTCTTCCTTTTTCAATATTATTGAAGCATTTATC
AGGGTTATTGTCTCATGAGCGGATACATATTTGAATGTATTTAGAAAAATAAACAAATAGGGGTTCCGCGCACATTTC
CCCGAAAAGTGCCACCTGACGTCGACGGATCGGGAGATCTCCCGATCCCCTATGGTGCACTCTCAGTACAATCTGCTC
TGATGCCGCATAGTTAAGCCAGTATCTGCTCCCTGCTTGTGTGTTGGAGGTCGCTGAGTAGTGCGCGAGCAAAATTTA
AGCTACAACAAGGCAAGGCTTGACCGACAATTGCATGAAGAATCTGCTTAGGGTTAGGCGTTTTGCGCTGCTTCGCGA
TGTACGGGCCAGATATACGCGTTGACATTGATTATTGACTAGTTATTAATAGTAATCAATTACGGGGTCATTAGTTCA
TAGCCCATATATGGAGTTCCGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTATTGACGTCAATGACGGTAA
ATGGCCCGCCTGGCATTATGCCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTTGACTCACGGGGATTTCCAAG
TCTCCACCCCATTGACGTCAATGGGAGTTTGTTTTGGCACCAAAATCAACGGGACTTTCCAAAATGTCGTAACAACTC
CGCCCCATTGACGCAAATGGGCGGTAGGCGTGTACGGTGGGAGGTCTATATAAGCAGAGCTCTCTGGCTAACTAGAGA
ACCCACTGCTTACTGGCTTATCGAAATTAATACGACTCACTATAGGGAGACCCAAGCTGGCTAGCGTTTAAACTTAAG
CTTGCCACCATGAACATCCCCGCTCTGGTGGAAAACCAGAAGAAGTACTTTGGCACCTACAGCGTGATGGCCATGCTG
AACGCTCAGACCGTGCTGGACCACATCCAGAAGGTGGCCGATATTGAGGGCGAGCAGAACGAGAACAACGAGAATCTG
TGGTTTCACCCCGTGATGAGCCACCTGTACAACGCCAAGAACGGCTACGACAAGCAGCCCGAGAAAACCATGTTCATC
ATCGAGCGGCTGCAGAGCTACTTCCCATTCCTGAAGATCATGGCCGAGAACCAGAGAGAGTACAGCAACGGCAAGTAC
AAGCAGAACCGCGTGGAAGTGAACAGCAACGACATCTTCGAGGTGCTGAAGCGCGCCTTCGGCGTGCTGAAGATGTAC
AGGGACCTGACCAACGCATACAAGACCTACGAGGAAAAGCTGAACGACGGCTGCGAGTTCCTGACCAGCACAGAGCAA
CCTCTGAGCGGCATGATCAACAACTACTACACAGTGGCCCTGCGGAACATGAACGAGAGATACGGCTACAAGACAGAG
GACCTGGCCTTCATCCAGGACAAGCGGTTCAAGTTCGTGAAGGACGCCTACGGCAAGAAAAAGTCCCAAGTGAATACC
GGATTCTTCCTGAGCCTGCAGGACTACAACGGCGACACACAGAAGAAGCTGCACCTGAGCGGAGTGGGAATCGCCCTG
CTGATCTGCCTGTTCCTGGACAAGCAGTACATCAACATCTTTCTGAGCAGGCTGCCCATCTTCTCCAGCTACAATGCC
CAGAGCGAGGAACGGCGGATCATCATCAGATCCTTCGGCATCAACAGCATCAAGCTGCCCAAGGACCGGATCCACAGC
GAGAAGTCCAACAAGAGCGTGGCCATGGATATGCTCAACGAAGTGAAGCGGTGCCCCGACGAGCTGTTCACAACACTG
TCTGCCGAGAAGCAGTCCCGGTTCAGAATCATCAGCGACGACCACAATGAAGTGCTGATGAAGCGGAGCAGCGACAGA
TTCGTGCCTCTGCTGCTGCAGTATATCGATTACGGCAAGCTGTTCGACCACATCAGGTTCCACGTGAACATGGGCAAG
CTGAGATACCTGCTGAAGGCCGACAAGACCTGCATCGACGGCCAGACCAGAGTCAGAGTGATCGAGCAGCCCCTGAAC
GGCTTCGGCAGACTGGAAGAGGCCGAGACAATGCGGAAGCAAGAGAACGGCACCTTCGGCAACAGCGGCATCCGGATC
AGAGACTTCGAGAACATGAAGCGGGACGACGCCAATCCTGCCAACTATCCCTACATCGTGGACACCTACACACACTAC
ATCCTGGAAAACAACAAGGTCGAGATGTTTATCAACGACAAAGAGGACAGCGCCCCACTGCTGCCCGTGATCGAGGAT
GATAGATACGTGGTCAAGACAATCCCCAGCTGCCGGATGAGCACCCTGGAAATTCCAGCCATGGCCTTCCACATGTTT
CTGTTCGGCAGCAAGAAAACCGAGAAGCTGATCGTGGACGTGCACAACCGGTACAAGAGACTGTTCCAGGCCATGCAG
AAAGAAGAAGTGACCGCCGAGAATATCGCCAGCTTCGGAATCGCCGAGAGCGACCTGCCTCAGAAGATCCTGGATCTG
ATCAGCGGCAATGCCCACGGCAAGGATGTGGACGCCTTCATCAGACTGACCGTGGACGACATGCTGACCGACACCGAG
CGGAGAATCAAGAGATTCAAGGACGACCGGAAGTCCATTCGGAGCGCCGACAACAAGATGGGAAAGAGAGGCTTCAAG
CAGATCTCCACAGGCAAGCTGGCCGACTTCCTGGCCAAGGACATCGTGCTGTTTCAGCCCAGCGTGAACGATGGCGAG
AACAAGATCACCGGCCTGAACTACCGGATCATGCAGAGCGCCATTGCCGTGTACGATAGCGGCGACGATTACGAGGCC
AAGCAGCAGTTCAAGCTGATGTTCGAGAAGGCCCGGCTGATCGGCAAGGGCACAACAGAGCCTCATCCATTTCTGTAC
AAGGTGTTCGCCCGCAGCATCCCCGCCAATGCCGTCGAGTTCTACGAGCGCTACCTGATCGAGCGGAAGTTCTACCTG
ACCGGCCTGTCCAACGAGATCAAGAAAGGCAACAGAGTGGATGTGCCCTTCATCCGGCGGGACCAGAACAAGTGGAAA
ACACCCGCCATGAAGACCCTGGGCAGAATCTACAGCGAGGATCTGCCCGTGGAACTGCCCAGACAGATGTTCGACAAT
GAGATCAAGTCCCACCTGAAGTCCCTGCCACAGATGGAAGGCATCGACTTCAACAATGCCAACGTGACCTATCTGATC
GCCGAGTACATGAAGAGAGTGCTGGACGACGACTTCCAGACCTTCTACCAGTGGAACCGCAACTACCGGTACATGGAC
ATGCTTAAGGGCGAGTACGACAGAAAGGGCTCCCTGCAGCACTGCTTCACCAGCGTGGAAGAGAGAGAAGGCCTCTGG
AAAGAGCGGGCCTCCAGAACAGAGCGGTACAGAAAGCAGGCCAGCAACAAGATCCGCAGCAACCGGCAGATGAGAAAC
GCCAGCAGCGAAGAGATCGAGACAATCCTGGATAAGCGGCTGAGCAACAGCCGGAACGAGTACCAGAAAAGCGAGAAA
GTGATCCGGCGCTACAGAGTGCAGGATGCCCTGCTGTTTCTGCTGGCCAAAAAGACCCTGACCGAACTGGCCGATTTC
GACGGCGAGAGGTTCAAACTGAAAGAAATCATGCCCGACGCCGAGAAGGGAATCCTGAGCGAGATCATGCCCATGAGC
TTCACCTTCGAGAAAGGCGGCAAGAAGTACACCATCACCAGCGAGGGCATGAAGCTGAAGAACTACGGCGACTTCTTT
GTGCTGGCTAGCGACAAGAGGATCGGCAACCTGCTGGAACTCGTGGGCAGCGACATCGTGTCCAAAGAGGATATCATG
GAAGAGTTCAACAAATACGACCAGTGCAGGCCCGAGATCAGCTCCATCGTGTTCAACCTGGAAAAGTGGGCCTTCGAC
ACATACCCCGAGCTGTCTGCCAGAGTGGACCGGGAAGAGAAGGTGGACTTCAAGAGCATCCTGAAAATCCTGCTGAAC
AACAAGAACATCAACAAAGAGCAGAGCGACATCCTGCGGAAGATCCGGAACGCCTTCGATGCAAACAATTACCCCGAC
AAAGGCGTGGTGGAAATCAAGGCCCTGCCTGAGATCGCCATGAGCATCAAGAAGGCCTTTGGGGAGTACGCCATCATG
AAGGGAAGCCTGCAGCTGCCTCCACTTGAAAGACTGACACTGGGATCCGGCAAGCCCATCCCTAACCCACTGCTGGGC
CTGGACAGCACCCTCGAgAACCCCAGTGCCCCCAGCTACCCGATGGCCTCGCTCTACGTGGGGGACCTCCACCCCGAC
GTGACCGAGGCGATGCTCTACGAGAAGTTCAGCCCGGCCGGGCCCATCCTCTCCATCCGGGTCTGCAGGGACATGATC
ACCCGCCGCTCCTTGGGCTACGCGTATGTGAACTTCCAGCAGCCGGCGGACGCGGAGCGTGCTTTGGACACCATGAAT
TTTGATGTTATAAAGGGCAAGCCAGTACGCATCATGTGGTCTCAGCGTGATCCATCACTTCGCAAAAGTGGAGTAGGC
AACATATTCATTAAAAATCTGGACAAATCCATTGATAATAAAGCACTGTATGATACATTTTCTGCTTTTGGTAACATC
CTTTCATGTAAGGTGGTTTGTGATGAAAATGGTTCCAAGGGCTATGGATTTGTACACTTTGAGACGCAGGAAGCAGCT
GAAAGAGCTATTGAAAAAATGAATGGAATGCTCCTAAATGATCGCAAAGTATTTGTTGGACGATTTAAGTCTCGTAAA
GAACGAGAAGCTGAACTTGGAGCTAGGGCAAAAGAATTCACCAATGTTTACATCAAGAATTTTGGAGAAGACATGGAT
GATGAGCGCCTTAAGGATCTCTTTGGCAAGTTTGGGCCTGCCTTAAGTGTGAAAGTAATGACTGATGAAAGTGGAAAA
TCCAAAGGATTTGGATTTGTAAGCTTTGAAAGGCATGAAGATGCACAGAAAGCTGTGGATGAGATGAACGGAAAGGAG
CTCAATGGAAAACAAATTTATGTTGGTCGAGCTCAGAAAAAGGTGGAACGGCAGACGGAACTTAAGCGCAAATTTGAA
CAGATGAAACAAGATAGGATCACCAGATACCAGGGTGTTAATCTTTATGTGAAAAATCTTGATGATGGTATTGATGAT
GAACGTCTCCGGAAAGAGTTTTCTCCATTTGGTACAATCACTAGTGCAAAGGTTATGATGGAAGGTGGTCGCAGCAAA
GGGTTTGGTTTTGTATGTTTCTCCTCCCCAGAAGAAGCCACTAAAGCAGTTACAGAAATGAACGGTAGAATTGTGGCC
ACAAAGCCATTGTATGTAGCTTTAGCTCAGCGCAAAGAAGAGCGCCAGGCTCACCTCACTAACCAGTATATGCAGAGA
ATGGCAAGTGTACGAGCTGTTCCCAACCCTGTAATCAACCCCTACCAGCCAGCACCTCCTTCAGGTTACTTCATGGCA
GCTATCCCACAGACTCAGAACCGTGCTGCATACTATCCTCCTAGCCAAATTGCTCAACTAAGACCAAGTCCTCGCTGG
ACTGCTCAGGGTGCCAGACCTCATCCATTCCAAAATATGCCCGGTGCTATCCGCCCAGCTGCTCCTAGACCACCATTT
AGTACTATGAGACCAGCTTCTTCACAGGTTCCACGAGTCATGTCAACACAGCGTGTTGCTAACACATCAACACAGACA
ATGGGTCCACGTCCTGCAGCTGCAGCCGCTGCAGCTACTCCTGCTGTCCGCACCGTTCCACAGTATAAATATGCTGCA
GGAGTTCGCAATCCTCAGCAACATCTTAATGCACAGCCACAAGTTACAATGCAACAGCCTGCTGTTCATGTACAAGGT
CAGGAACCTTTGACTGCTTCCATGTTGGCATCTGCCCCTCCTCAAGAGCAAAAGCAAATGTTGGGTGAACGGCTGTTT
CCTCTTATTCAAGCCATGCACCCTACTCTTGCTGGTAAAATCACTGGCATGTTGTTGGAGATTGATAATTCAGAACTT
CTTCACATGCTGGAGTCTCCAGAGTCACTCCGTTCTAAGGTTGATGAAGCTGTAGCTGTACTACAAGCCCACCAAGCT
AAAGAGGCTGCCCAGAAAGCAGTTAACAGTGCCACCGGTGTTCCAACTGTTTAAGCGGCCGCTCGAGCCTAGAGGGCC
CGTTTAAACCCGCTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCTAGGGGGTATCCCCACGCGCCCTGTAGCGG
CGCATTAAGCGCGGCGGGTGTGGTGGTTACGCGCAGCGTGACCGCTACACTTGCCAGCGCCCTAGCGCCCGCTCCTTT
CGCTTTCTTCCCTTCCTTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTAGGGTGATGGTTCACGTAGTGGGCCATCGCCCTG
ATAGACGGTTTTTCGCCCTTTGACGTTGGAGTCCACGTT
SEQ ID NO: 51 Nuclear export signal
LPPLERLTL
SEQ ID NO: 52 >pJC1212 (2963 bp)
AGTGGCTGCTGCCAGTGGCGATAAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACACCGAACTGAGATACCTACAGCG
TGAGCTATGAGAAAGCGCCACGCTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGAGCGAGGAAGCGGAAGAGCGCCC
AATACGCAAACCGCCTCTCCCCGCGCGTTGGCCGATTCATTAATGCAGCTGGCACGACAGGTTTCCCGACTGGAAAGC
GGGCAGTGAGCGCAACGCAATTAATGTGAGTTAGCTCACTCATTAGGCACCCCAGGCTTTACACTTTATGCTTCCGGC
TCGTATGTTGTGTGGAATTGTGAGCGGATAACAATTTCACGCAGGAAACAGCTATGACCATGATTACGCCAGAGGGCC
TATTTCCCATGATTCCTTCATATTTGCATATACGATACAAGGCTGTTAGAGAGATAATTGGAATTAATTTGACTGTAA
ACACAAAGATATTAGTACAAAATACGTGACGTAGAAAGTAATAATTTCTTGGGTAGTTTGCAGTTTTAAAATTATGTT
TTAAAATGGACTATCATATGCTTACCGTAACTTGAAAGTATTTCGATTTCTTGGCTTTATATATCTTGTGGAAAGGAC
GAAACACCgcactatagaatagggccctctagGTTGTGGAAGGTCCAGTTTTGAGGGGCTATTACAACTTTTTTGGTA
CCGAGCTCGAATTCACTGGCCGTCGTTTTACAACGTCGTGACTGGGAAAACCCTGGCGTTACCCAACTTAATCGCCTT
GCAGCACATCCCCCTTTCGCCAGCTGGCGTAATAGCGAAGAGGCCCGCACCGATCGCCCTTCCCAACAGTTGCGCAGC
CTGAATGGCGAATGGCGCCTGATGCGGTATTTTCTCCTTACGCATCTGTGCGGTATTTCACACCGCATATGGTGCACT
CTCAGTACAATCTGCTCTGATGCCGCATAGTTAAGCCAGCCCCGACACCCGCCAACACCCGCTGACGCGCCCTGACGG
GCTTGTCTGCTCCCGGCATCCGCTTACAGACAAGCTGTGACCGTCTCCGGGAGCTGCATGTGTCAGAGGTTTTCACCG
TCATCACCGAAACGCGCGAGACGAAAGGGCCTCGTGATACGCCTATTTTTATAGGTTAATGTCATGATAATAATGGTT
TCTTAGACGTCAGGTGGCACTTTTCGGGGAAATGTGCGCGGAACCCCTATTTGTTTATTTTTCTAAATACATTCAAAT
ATGTATCCGCTCATGAGACAATAACCCTGATAAATGCTTCAATAATATTGAAAAAGGAAGAGTATGAGTATTCAACAT
TTCCGTGTCGCCCTTATTCCCTTTTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCAACAGCGGTAAGATCCTTGAGAGT
TTTCGCCCCGAAGAACGTTTTCCAATGATGAGCACTTTTAAAGTTCTGCTATGTGGCGCGGTATTATCCCGTATTGAC
GCCGGGCAAGAGCAACTCGGTCGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGTGATAACACTGCGGCCAACTTA
CTTCTGACAACGATCGGAGGACCGAAGGAGCTAACCGCTTTTTTGCACAACATGGGGGATCATGTAACTCGCCTTGAT
CGTTGGGAACCGGAGCTGAATGAAGCCATACCAAACGACGAGCGTGACACCACGATGCCTGTAGCAATGGCAACAACG
TTGCGCAAACTATTAACTGGCGAACTACTTACTCTAGCTTCCCGGCAACAATTAATAGACTGGATGGAGGCGGATAAA
GTTGCAGGACCACTTCTGCGCTCGGCCCTTCCGGCTGGCTGGTTTATTGCTGATAAATCTGGAGCCGGTGAGCGTGGG
TCTCGCGGTATCATTGCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGGTAACTGTCAGACCAA
GTTTACTCATATATACTTTAGATTGATTTAAAACTTCATTTTTAATTTAAAAGGATCTAGGTGAAGATCCTTTTTGAT
AATCTCATGACCAAAATCCCTTAACGTGAGTTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAAATACTGTTCTTCTA
GTGTAGCCGTAGTTAGGCCACCACTTCAAGAACTCTGTAGCACCGCCTACATACCTCGCTCTGCTAATCCTGTTACC
SEQ ID NO: 53 >pJC1213 (2962 bp)
AGTGGCTGCTGCCAGTGGCGATAAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACACCGAACTGAGATACCTACAGCG
TGAGCTATGAGAAAGCGCCACGCTTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAGCAACGCGGCCTTTTTACGGTTCCT
GGCCTTTTGCTGGCCTTTTGCTCACATGTTCTTTCCTGCGTTATCCCCTGATTCTGTGGATAACCGTATTACCGCCTT
TGAGTGAGCTGATACCGCTCGCCGCAGCCGAACGACCGAGCGCAGCGAGTCAGTGAGCGAGGAAGCGGAAGAGCGCCC
AATACGCAAACCGCCTCTCCCCGCGCGTTGGCCGATTCATTAATGCAGCTGGCACGACAGGTTTCCCGACTGGAAAGC
GGGCAGTGAGCGCAACGCAATTAATGTGAGTTAGCTCACTCATTAGGCACCCCAGGCTTTACACTTTATGCTTCCGGC
TCGTATGTTGTGTGGAATTGTGAGCGGATAACAATTTCACGCAGGAAACAGCTATGACCATGATTACGCCAGAGGGCC
TATTTCCCATGATTCCTTCATATTTGCATATACGATACAAGGCTGTTAGAGAGATAATTGGAATTAATTTGACTGTAA
ACACAAAGATATTAGTACAAAATACGTGACGTAGAAAGTAATAATTTCTTGGGTAGTTTGCAGTTTTAAAATTATGTT
TTAAAATGGACTATCATATGCTTACCGTAACTTGAAAGTATTTCGATTTCTTGGCTTTATATATCTTGTGGAAAGGAC
GAAACACCgagctctagcatttaggtgacacGTTGTGGAAGGTCCAGTTTTGAGGGGCTATTACAACTTTTTTGGTAC
CGAGCTCGAATTCACTGGCCGTCGTTTTACAACGTCGTGACTGGGAAAACCCTGGCGTTACCCAACTTAATCGCCTTG
CAGCACATCCCCCTTTCGCCAGCTGGCGTAATAGCGAAGAGGCCCGCACCGATCGCCCTTCCCAACAGTTGCGCAGCC
TGAATGGCGAATGGCGCCTGATGCGGTATTTTCTCCTTACGCATCTGTGCGGTATTTCACACCGCATATGGTGCACTC
TCAGTACAATCTGCTCTGATGCCGCATAGTTAAGCCAGCCCCGACACCCGCCAACACCCGCTGACGCGCCCTGACGGG
CTTGTCTGCTCCCGGCATCCGCTTACAGACAAGCTGTGACCGTCTCCGGGAGCTGCATGTGTCAGAGGTTTTCACCGT
CATCACCGAAACGCGCGAGACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCAGAAACGCTGGTGAAAGTAA
AAGATGCTGAAGATCAGTTGGGTGCACGAGTGGGTTACATCGAACTGGATCTCAACAGCGGTAAGATCCTTGAGAGTT
TTCGCCCCGAAGAACGTTTTCCAATGATGAGCACTTTTAAAGTTCTGCTATGTGGCGCGGTATTATCCCGTATTGACG
CCGGGCAAGAGCAACTCGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAACACTGCGGCCAACTTAC
TTCTGACAACGATCGGAGGACCGAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCACGATGCCTGTAGCAATGGCAACAACGT
TGCGCAAACTATTAACTGGCGAACTACTTACTCTAGCTTCCCGGCAACAATTAATAGACTGGATGGAGGCGGATAAAG
TTGCAGGACCACTTCTGCGCTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATCTACACGACGGGGAGTCAGG
CAACTATGGATGAACGAAATAGACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAGGTGAAGATCCTTTTTGATA
ATCTCATGACCAAAATCCCTTAACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTACCAGCGGTGGTTTGTTTGC
CGGATCAAGAGCTACCAACTCTTTTTCCGAAGGTAACTGGCTTCAGCAGAGCGCAGATACCAAATACTGTTCTTCTAG
TGTAGCCGTAGTTAGGCCACCACTTCAAGAACTCTGTAGCACCGCCTACATACCTCGCTCTGCTAATCCTGTTACC
SEQ ID NO: 54 >addgene-plasmid-103854-sequence-254736 (2962 bp)
agtggctgctgccagtggcgataagtcgtgtcttaccgggttggactcaagacgatagttaccggataaggcgcagcg
gtcgggctgaacggggggttcgtgcacacagcccagcttggagcgaacgacctacaccgaactgagatacctacagcg
tgagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttga
gcgtcgatttttgtgatgctcgtcaggggggcggagcctatggaaaaacgccagcaacgcggcctttttacggttcct
ggccttttgctggccttttgctcacatgttctttcctgcgttatcccctgattctgtggataaccgtattaccgcctt
tgagtgagctgataccgctcgccgcagccgaacgaccgagcgcagcgagtcagtgagcgaggaagcggaagagcgccc
aatacgcaaaccgcctctccccgcgcgttggccgattcattaatgcagctggcacgacaggtttcccgactggaaagc
gggcagtgagcgcaacgcaattaatgtgagttagctcactcattaggcaccccaggctttacactttatgcttccggc
tcgtatgttgtgtggaattgtgagcggataacaatttcacgcaggaaacagctatgaccatgattacgccagagggcc
tatttcccatgattccttcatatttgcatatacgatacaaggctgttagagagataattggaattaatttgactgtaa
acacaaagatattagtacaaaatacgtgacgtagaaagtaataatttcttgggtagtttgcagttttaaaattatgtt
ttaaaatggactatcatatgcttaccgtaacttgaaagtatttcgatttcttggctttatatatcttgtggaaaggac
gaaacaccatgtcttcctgggacgaagacaagttgtggaaggtccagttttgaggggctattacaacttttttggtac
cgagctcgaattcactggccgtcgttttacaacgtcgtgactgggaaaaccctggcgttacccaacttaatcgccttg
cagcacatccccctttcgccagctggcgtaatagcgaagaggcccgcaccgatcgcccttcccaacagttgcgcagcc
tgaatggcgaatggcgcctgatgcggtattttctccttacgcatctgtgcggtatttcacaccgcatatggtgcactc
tcagtacaatctgctctgatgccgcatagttaagccagccccgacacccgccaacacccgctgacgcgccctgacggg
cttgtctgctcccggcatccgcttacagacaagctgtgaccgtctccgggagctgcatgtgtcagaggttttcaccgt
catcaccgaaacgcgcgagacgaaagggcctcgtgatacgcctatttttataggttaatgtcatgataataatggttt
cttagacgtcaggtggcacttttcggggaaatgtgcgcggaacccctatttgtttatttttctaaatacattcaaata
tgtatccgctcatgagacaataaccctgataaatgcttcaataatattgaaaaaggaagagtatgagtattcaacatt
tccgtgtcgcccttattcccttttttgcggcattttgccttcctgtttttgctcacccagaaacgctggtgaaagtaa
aagatgctgaagatcagttgggtgcacgagtgggttacatcgaactggatctcaacagcggtaagatccttgagagtt
ttcgccccgaagaacgttttccaatgatgagcacttttaaagttctgctatgtggcgcggtattatcccgtattgacg
ccgggcaagagcaactcggtcgccgcatacactattctcagaatgacttggttgagtactcaccagtcacagaaaagc
atcttacggatggcatgacagtaagagaattatgcagtgctgccataaccatgagtgataacactgcggccaacttac
ttctgacaacgatcggaggaccgaaggagctaaccgcttttttgcacaacatgggggatcatgtaactcgccttgatc
gttgggaaccggagctgaatgaagccataccaaacgacgagcgtgacaccacgatgcctgtagcaatggcaacaacgt
tgcgcaaactattaactggcgaactacttactctagcttcccggcaacaattaatagactggatggaggcggataaag
ttgcaggaccacttctgcgctcggcccttccggctggctggtttattgctgataaatctggagccggtgagcgtgggt
ctcgcggtatcattgcagcactggggccagatggtaagccctcccgtatcgtagttatctacacgacggggagtcagg
caactatggatgaacgaaatagacagatcgctgagataggtgcctcactgattaagcattggtaactgtcagaccaag
tttactcatatatactttagattgatttaaaacttcatttttaatttaaaaggatctaggtgaagatcctttttgata
atctcatgaccaaaatcccttaacgtgagttttcgttccactgagcgtcagaccccgtagaaaagatcaaaggatctt
cttgagatcctttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatactgttcttctag
tgtagccgtagttaggccaccacttcaagaactctgtagcaccgcctacatacctcgctctgctaatcctgttacc

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A fusion protein comprising:
an RNA effector protein that targets mRNA(s) of an active allele of a gene associated with a haploinsufficiency disorder; and
a regulon moiety that stimulates and/or stabilizes the mRNA(s).

2. The fusion protein of claim 1, wherein the RNA effector protein is a Cas effector protein selected from the group consisting of Cas9, Cas12, Cas13, and Cas14.

3. The fusion protein of claim 2, wherein the RNA effector protein is Cas13b.

4. The fusion protein of claim 2 or claim 3, wherein the Cas effector protein is a catalytically inactive Cas protein.

5. The fusion protein of any one of the preceding claims, wherein the regulon moiety is PABPC1 or NAT10.

6. The fusion protein of any one of the preceding claims, further comprising a linker and/or a spacer.

7. The fusion protein of any one of the preceding claims, further comprising a nuclear export signal and/or an epitope tag.

8. The fusion protein of any one of the preceding claims, wherein the RNA effector protein is N terminal to the regulon moiety.

9. The fusion protein of any one of the preceding claims, wherein the RNA effector protein is C terminal to the regulon moiety.

10. The fusion protein of claim 1 comprising or consisting of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

11. A polynucleotide encoding the fusion protein of any one of the preceding claims.

12. A vector comprising the polynucleotide of claim 11.

13. A cell comprising the vector of claim 12.

14. A system comprising:
a fusion protein comprising:
an RNA effector protein that targets mRNA(s) of an active allele of a gene associated with a haploinsufficiency disorder; and
a regulon moiety that stimulates and/or stabilizes the mRNA(s); and
a gRNA that forms a complex with the RNA effector protein and comprises a complementarity region that hybridizes with the mRNA(s) of the active allele.

15. The system of claim 13, wherein the RNA effector protein is a Cas effector protein selected from the group consisting of Cas9, Cas12, Cas13, and Cas14.

16. The system of claim 14, wherein the Cas effector protein is Cas13b.

17. The system of claim 14 or claim 15, wherein the Cas effector protein is a catalytically inactive Cas effector protein.

18. The system of any one of the preceding claims, wherein the regulon moiety is PABPC1 or NAT10.

19. The system of any one of the preceding claims, wherein the fusion protein further comprises a linker and/or a spacer.

20. The system of any one of the preceding claims, wherein the fusion protein further comprises a nuclear export signal and/or an epitope tag.

21. The system of any one of the preceding claims, wherein the RNA effector protein is N terminal to the regulon moiety.

22. The system of any one of the preceding claims, wherein the RNA effector protein is C terminal to the regulon moiety.

23. The system of any one of the preceding claims, wherein the fusion protein comprises or consists of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

24. The system of any one of the preceding claims, wherein the gRNA targets an mRNA encoding MeCP2, SCN1A, SYNGAP1, SHANK3, CHD2, or PTEN.

25. The system of any one of the preceding claims, wherein the gRNA comprises a complementarity region designed to hybridize to an mRNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof.

26. The system of any one of the preceding claims, wherein the gRNA comprises a complementarity region designed to hybridize to an mRNA selected from the group consisting of SEQ ID NO: 4, SEQ ID NO: 6, SEQ ID NO: 10, SEQ ID NO: 15, SEQ ID NO: 17, and combinations thereof.

27. The system of any one of the preceding claims, wherein the gRNA comprises a complementarity region designed to hybridize to an mRNA encoding an amino acid selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

28. The system of any one of the preceding claims, wherein the gRNA comprises a complementarity region designed to hybridize to an mRNA selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

29. The system of any one of the preceding claims, wherein the gRNA comprises a complementarity region designed to hybridize to an mRNA encoding an amino acid selected from the group consisting of SEQ ID NO: 37, SEQ ID NO: 38, SEQ ID NO: 39, SEQ ID NO: 40, SEQ ID NO: 41, and combinations thereof.

30. The system of any one of the preceding claims, wherein the gRNA is selected from the group consisting of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and SEQ ID NO: 72.

31. One or more polynucleotide(s) encoding the system of any one of the preceding claims.

32. One or more vector(s) comprising the polynucleotide(s) of claim 31.

33. A cell comprising the vector(s) of claim 32.

34. A complex comprising:
a fusion protein comprising:
an RNA effector protein that targets mRNA(s) of an active allele of a gene associated with a haploinsufficiency disorder; and
a regulon moiety that stimulates and/or stabilizes the mRNA(s), bound to a gRNA comprising a complementarity region that hybridizes with the mRNA(s) of the active allele.

35. The complex of claim 34, wherein the RNA effector protein is dCas13b and the regulon moiety is PABP1 or NAT10.

36. The complex of claim 34 or 35, wherein the gRNA comprises a complementarity region designed to hybridize to an mRNA selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof.

37. The complex of claim 34 or 35, wherein the gRNA comprises a complementarity region designed to hybridize to an mRNA selected from the group consisting of SEQ ID NO: 4, SEQ ID NO: 6, SEQ ID NO: 10, SEQ ID NO: 15, SEQ ID NO: 17, and combinations thereof.

38. The complex of claim 34 or 35, wherein the gRNA comprises a complementarity region designed to hybridize to an mRNA encoding an amino acid selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

39. The complex of claim 34 or 35, wherein the gRNA comprises a complementarity region designed to hybridize to an mRNA selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

40. The complex of claim 34 or 35, wherein the gRNA comprises a complementarity region designed to hybridize to an mRNA encoding SEQ ID NO: 37, SEQ ID NO: 38, SEQ ID NO: 39, SEQ ID NO: 40, SEQ ID NO: 41, and combinations thereof.

41. A complex comprising:
a fusion protein comprising:
an RNA effector protein that targets mRNA(s) of an active allele of a gene associated with a haploinsufficiency disorder; and
a regulon moiety that stimulates and/or stabilizes the mRNA(s), bound to a gRNA and the mRNA.

42. The complex of claim 41, wherein the RNA effector protein is dCas13b and the regulon moiety is PABP1 or NAT10.

43. The complex of claim 41 or 42, wherein the mRNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and combinations thereof.

44. The complex of claim 41 or 42, wherein the mRNA encodes an amino acid selected from the group consisting of SEQ ID NO: 4, SEQ ID NO: 6, SEQ ID NO: 10, SEQ ID NO: 15, SEQ ID NO: 17, and combinations thereof.

45. The complex of claim 41 or 42, wherein the mRNA is selected from the group consisting of SEQ ID NO: 19, SEQ ID NO: 20, SEQ ID NO: 21, SEQ ID NO: 22, SEQ ID NO: 23, SEQ ID NO: 24, and combinations thereof.

46. The complex of claim 41 or 42, wherein the mRNA is selected from the group consisting of SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, and combinations thereof.

47. The complex of claim 41 or 42, wherein the mRNA encodes an amino acid selected from the group consisting of is selected from the group consisting of SEQ ID NO: 37, SEQ ID NO: 38, SEQ ID NO: 39, SEQ ID NO: 40, SEQ ID NO: 41, and combinations thereof.

48. A pharmaceutical composition comprising:
the fusion protein or system of any one of the preceding claims.

49. The pharmaceutical composition of claim 48, further comprising a pharmaceutically acceptable carrier.

50. A pharmaceutical composition comprising:
one or more nucleic acids encoding the fusion protein or system of any one of the preceding claims.

51. A viral vector comprising one or more nucleic acids encoding the fusion protein or system of any one of the preceding claims.

52. The viral vector of claim 51, wherein the viral vector is an adeno-associated viral vector.

53. A nanoparticle or liposome comprising the fusion protein or system of any one of the preceding claims or one or more nucleic acids encoding the fusion protein or system of any one of the preceding claims.

54. A method of stimulating or stabilizing mRNA(s), the method comprising:
contacting the mRNA(s) with the fusion protein or system of any one of the preceding claims.

55. The method of claim 54, wherein the method is carried out, in vitro, in vivo, or ex vivo.

56. A method of treating or preventing a haploinsufficiency disorder in a subject, the method comprising:
administering to the subject a fusion protein or a nucleic acid encoding a fusion protein comprising:
an RNA effector protein that targets mRNA(s) of an active allele of a gene associated with a haploinsufficiency disorder; and
a regulon moiety that stimulates and/or stabilizes the mRNA(s); and
a gRNA or a nucleic acid encoding a gRNA designed to form a complex with the RNA effector protein and comprising a complementarity region designed to hybridize with the mRNA of the active allele.

57. The method of claim 56, wherein the RNA effector protein is a Cas effector protein selected from the group consisting of a Cas9, Cas12, Cas13, and Cas14.

58. The method of claim 57, wherein the Cas effector protein is Cas13b.

59. The method of claim 57 or 58, wherein the Cas effector protein is a catalytically inactive Cas protein.

60. The method of any one of the preceding claims, wherein the regulon moiety is PABPC1 or NAT10.

61. The method of any one of the preceding claims, wherein the fusion protein further comprises a linker and/or a spacer.

62. The method of any one of the preceding claims, wherein the fusion protein further comprises a nuclear export signal and/or an epitope tag.

63. The method of any one of the preceding claims, wherein the RNA effector protein is N terminal to the regulon moiety.

64. The method of any one of the preceding claims, wherein the RNA effector protein is C terminal to the regulon moiety.

65. The method of any one of the preceding claims, wherein the fusion protein comprises or consists of SEQ ID NO: 48 or SEQ ID NO: 49 or a polypeptide having at least 80%, at least 85%, at least 90%, at least 95%, or at least 99% sequence identity to SEQ ID NO: 48 or SEQ ID NO: 49.

66. The method of any one of the preceding claims, wherein the haploinsufficiency disorder is selected from the group consisting from 5qsyndrome, Adams-Oliver syndrome 1, Adams-Oliver syndrome 3, Adams-Oliver syndrome 5, Adams-Oliver syndrome 6, Alagille syndrome 1, Autoimmune lymphoproliferative syndrome type IA, Autoimmune lymphoproliferative syndrome type V, Autosomal dominant deafness-2A,Brain malformations with or without urinary tract defects (BRMUTD), Carney complex type 1,CHARGE syndrome, Cleidocranial dysplasia, Currarino syndrome, Denys-Drash syndrome/Frasier syndrome, Developmental delay, intellectual disability, obesity, and dysmorphic features (DIDOD), DiGeorge syndrome (TBXI-associated), Dravet syndrome, Duane-radial raysyndrome, Ehlers-Danlos syndrome (classic-like), Ehlers-Danlos syndrome (vascular type), Feingold syndrome 1, Frontotemporal lobar degeneration with TDP43 inclusions (FTLD-TDP), GRN-related, GLUT I deficiency syndrome, Greig cephalopolysyndactyly syndrome, Hereditary hemorrhagic telangiectasia type 1, Holoprosencephaly 3, Holoprosencephaly 4, Holoprosencephaly 5, Holt-Oram syndrome, Hypoparathyroidism, sensorineural deafness, adrenal disease (HDR), Kleefstra syndrome 1, Klippel-Trenaunay syndrome (AAGF-related), Leri-Weill dyschondrosteosis, Marfan syndrome, Mental retardation and distinctive facial features with or without cardiac defects (MRFACD), Mental retardation, autosomal dominant 1, Mental retardation, autosomal dominant 19, Mental retardation, autosomal dominant 29, Nail-patella syndrome (NPS), Phelan-McDermid syndrome, Pitt-Hopkins syndrome, Primary pulmonary hypertension 1, Rett syndrome (congenital variant), Smith-Magenis syndrome (RAII associated), Sotos syndrome 1, Sotos syndrome 2, Stickler syndrome type I, Supravalvular aorticstenosis, SYNGAPI-related intellectual disability, Treacher Collins syndrome, Trichorhinophalangeal syndrome type I, Ulnar-mammary syndrome, van der Woude syndrome1, Waardenburg syndrome type 1, Waardenburg syndrome type 2A, and Waardenburg syndrometype 4C.

67. The method of any one of the preceding claims, wherein the haploinsufficiency disorder is a CNS haploinsufficiency disorder.

68. The method of claim 67, wherein the CNS haploinsufficiency disorder is selected from the group consisting of episodic ataxia, familial hemiplegia migraine, CDKL5 deficiency disorder, CHD2 myoclonic encephalopathy, familial focal epilepsy with variable loci, FOXG1 syndrome, benign familial neonatal seizures, Rett syndrome, Dravat syndrome, SCN2A-epileptic encephalopathy, SCN2A-developmental encephalopathy, SCN8A-epileptic encephalopathy, SC8A familial infantile epilepsy, early infantile epileptic encephalopathy, myoclonic-atonic epilepsy, early infantile epileptic encephalopathy, SYNGAP1-related intellectual disability, tuberous sclerosis, Lennox-Gastaut Syndrome, FoxG1 syndrome, KCNQ2-related epileptic encephalopathy, PCDH19-related epilepsy, SLC6A1-related myoclonic-astatic epilepsy, STXBP1-related epileptic encephalopathy, SYNGAP1 syndrome, and combinations thereof.

69. The method of any one of the preceding claims, wherein the haploinsufficiency disorder is Dravet Syndrome or Rett syndrome.

70. The method of any one of the preceding claims, wherein the subject has a haploinsufficiency in a gene selected from the group consisting of AGGFI, ARHGAP 31, BMPR2, CHD7, COL2Al, COL3Al, CTLA4, CTNNBI, DLL4, EHMTI, ELN, ENG, FAS, FBNI, FOXG1, GATA3, GLI3, GRN, IRF6, JAGI, KCNQ4, LMXIB, MBD5, MED13L, MITF, MNXI, MYCN, NFIA, NFIX, NOTCH!, NSDI, PAX3, PHIP, PRKARIA, RAil, RBPJ, RPS14, RUNX2, SALL4, SCN1A, SETBPI, SHANK3, SHH, SHOX, SLC2Al/GLUT1, SOXI0, SYNGAPI, TBXI, TBX3, TBX5, TCF4, TCOFI, TGIFI, TNXB, TRPSI, WTI, ZIC2, and combinations thereof.

71. The method of any one of the preceding claims, wherein the subject has a haploinsufficiency in a gene selected from the group consisting of SCN1A, SCN2A, SCN8A, SCN12A5, SPTAN1, CDKL5, CHD2, FOXG1, KCNQ2, PCDH19, SLC6A1, STXBP1, SYNGAP1, CACNA1A, DEPDC5, MECP2, TSC1, TSC2, and combinations thereof.

72. The method of any one of the preceding claims, wherein the subject has mutation selected from the list in Table 4 and combinations thereof.

73. The method of any one of the preceding claims, wherein the subject has a mutation selected from the list in Table 6 and combinations thereof.

74. The method of any one of the preceding claims, wherein the subject is a mammal.

75. The method of claim 74, wherein the subject is a human.

76. The method of any one of the preceding claims, wherein the fusion protein and gRNA are administered as part of a pharmaceutical composition.

77. The method of any one of the preceding claims, wherein administering comprises administering a viral vector comprising nucleic acid sequence(s) encoding the fusion protein and gRNA to the subject.

78. The method of any one of the preceding claims, wherein administering comprises administering a nanoparticle or liposome comprising the fusion protein and gRNA or nucleic acid sequence(s) encoding the fusion protein and gRNA to the subject.


SEQUENCE LISTING (89 sequences)

SEQ ID NO: 1 - DNA, 13051 bp, Homo sapiens
  source 1..13051: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggataatcttgctccaacttggatggggtggagcgctggttcctcccctgagccctttattatgggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 2 - DNA, 8537 bp, Homo sapiens
  source 1..8537: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggataatcttgctccaacttggatggggtggagcgctggttcctcccctgagccctttattatgggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 3 - DNA, 8565 bp, Homo sapiens
  source 1..8565: mol_type = genomic DNA
agagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggataatcttgctccaacttggatggggtggagcgctggttcctcccctgagccctttattatgggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 4 - DNA, 12765 bp, Homo sapiens
  source 1..12765: mol_type = genomic DNA
aagtgtaggagacacactgctggcctgtggaaactcatggaactgttcctccagattaacacttcaggggttatggaagctggaggaagctgagcttttactacatcttttgggggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 5 - DNA, 12806 bp, Homo sapiens
  source 1..12806: mol_type = genomic DNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 6 - DNA, 12857 bp, Homo sapiens
  source 1..12857: mol_type = genomic DNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 7 - DNA, 12890 bp, Homo sapiens
  source 1..12890: mol_type = genomic DNA
aagtgtaggagacacactgctggcctgtggaaactcatggaactgttcctccagattaacacttcaggggttatggaagctggaggaagctgagcttttactacatcttttgggggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 8 - DNA, 12897 bp, Homo sapiens
  source 1..12897: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 9 - DNA, 12849 bp, Homo sapiens
  source 1..12849: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 10 - DNA, 12900 bp, Homo sapiens
  source 1..12900: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 11 - DNA, 12933 bp, Homo sapiens
  source 1..12933: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 12 - DNA, 12938 bp, Homo sapiens
  source 1..12938: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 13 - DNA, 12989 bp, Homo sapiens
  source 1..12989: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 14 - DNA, 12941 bp, Homo sapiens
  source 1..12941: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttgggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 15 - DNA, 12992 bp, Homo sapiens
  source 1..12992: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 16 - DNA, 13008 bp, Homo sapiens
  source 1..13008: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggtaagcatattggaaggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 17 - DNA, 13079 bp, Homo sapiens
  source 1..13079: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaattgggtactgctgatagtactattcttcctggcacatggaaatatcccactgaggttgtcaatcaatattgatttaatcagtgtggcagcaaaggcactagaggaggaaaatactctagacaatcaagtatagatgggaaaattgcatcaccaagaaattcttaaaatcacaagtggataatttgcaatatcaattgattttactcaactgcttgagtcctttatcattccgttagaacactcctcatatttgcagataactcagcccctcacctcagcaacaagcaagaggacatcattataaacttcctcaatttctttcctatccagtgaaaaatatctaagtcccttttcccttcccttttccttcccttcccttcctttttctttccttccattctcttcctttttccttcctttttctttccttccattctcttcctttttccttcctttttcttactttacctccccctcctacctttccctttctcccttcctccctctctcccttccttcctctctccctctccttccttccttccttcaatccatacttttatatcttttcagagatcaatttttcccattcatttctacattctcagataccttgctccatgacttgttctacttctcttctatctttattctttccctcttgactgatcctttttgtctgcctttgcattttcaaaatcagtttttctaatcttaaaaaataactttgctcaaccctgatgcatctttattcactgtgtcattatctcattatttttaaaggttagtttctaaagccagggctctatactcactaccttcactcttggttaaaattaaaagaaatataggtcccagtgatatttatgggaaaatttgactgtgaataatcttgttgatataaacactttgggttttcaaaatgttatgctagctgttaactttcccagtaacattatgagggaagggctggaaaatacaaaaggtagacataggaaccgagaacaccacccctatggagccaagctatacatactctgtctctggttatcataaatacatctcctatattcaatgcttgcttagaaaggcatgatgtccacggagcaattcaattgttcactgtgatgggtttttttgttttctcctcatggaatgcaacttttatttat

SEQ ID NO: 18 - DNA, 11940 bp, Homo sapiens
  source 1..11940: mol_type = genomic DNA
accatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttattttattgtttgcccagtcactttttgttaacagaatattctaatgatatggagattttttacattacaaattgggggagaaggggagcgcgcgcgcacacacacacacacacacacacacacacacacacacacagaggcatacccacgttgacaacaaaacctagggtagatatgtcactggaggtagggggtaatgacctcccagaattacaagcagcaggtgtgttctctgttaggaggaagaactggtgtcagaggatagctagtgattctaggaggaagagaagtatggaagccagagtgatggtggatgaccccttgagctatgaaaagaaacccttaaatcatcatttaaaaatttagaattgccatgtgtgtaggatactgtgtttgctcctccagagccactctctctgcttctgcatcattctgtgtgtcccagaagggtgacttctacacattgcaaaaatgggctctcctacctttgagctcccaattggtttggccaatgagaagcaccagtgggaaagcaccagagagagaagattgacataggaatatttcttctccaattccttctttgctgggttggcactggactcattcctccccgaaaagtcatactccaatcagactgcccctcatacaactgaagctactttctctggggtcaggtaatcactcctccccttgctccttcaggtctgctgctgcattgagagtgcttttgtattccttgtagctttctcctaacattgctgacacttttgtaaatgtccccttcatgaaattcttctatatgcctcatttcagcatgccatctgtctcctgcctggctgacacaaggtgattcaacagctcatgaaagtcagcaggaagcaaagatgtgccttgcttcagcttggggtcttaatcttgctaacttttgcagataaagaaaaacagtaactgggggaaccacagtgaagtccagtgcagaattcacagatatcatggaaaggttactcgggtggtccagatagtaaaattaacagtctaaattaatctatctaaatttctgaggaacgagaagccttcccttgtcatcaggtgaagccagaagagggaatatagcctcaaccagaaaagggacagtaattaaaaggcttttcccatccttgtacaatggactgactttgcctcttcataacatcacaatcctaaagcaacacaacaattaattctgatatattagtagctgaaaaaaattcccatttccaactaaggtaggtcagaattataggataaaccctgcagactttttatactacccatccacgccattactcactgttacctttccaaatacaaagagaagaactggtaaaacataatcatataaatctccatattcattttgaaatatttggcatgatattttctgtgctaaaaagtaattattcttcaaagaatgatgaggtcatgtcagtaagacacaggaaccaactagaaggggcttcccactggccaaatctggggcaagttgagcatcaaaataaatgatagtaaaagattataattcattgaataagaatcagcaaatacatactgatgtaagtaaataaggaaaagtacaaatctgtttcttgcagttgaatgttaattaacaattgtagaagaaataacggagttagaaaaatcactatttggcaatcaccctaatgacaattgattcatacaagaatcatcaatgagtattaaaactcatgggtgaaagtttgatgaggaatagggtatttatagcatcttaaagtatctcttctctattaagtagaaaatttaaacagaagaaagtatactttggagaaatacagcagacaataccttcaaagatatcatcaattatgagaccaactgatactatgtgcctcctgataagatatactgaaagggccacattacttcttggtacacagtcaaaattttaaaaccagaatctaactacaaggaaaatcaaattgaggacactctataaaataagtggactgaactccttaaaaatgtcaatgtcatgaaagacaaagaaaggctaaagaattccatgaggtcaaagaactatgacaactaaacacaattctggatggaatatcaaattaaaaaataacagataaataatattattgggaaagttgaataaatttgaatatggactgtttattagttattagtattataatagtgttaattttcctaattttgttaagactagtgtgcctgttccatgaaaatagaaaatgttcttattctctgaaaatgcatgctaaagtatttaggggtgaatgcaacaatgtctgcagctcattcttgaatcagttcaaagaaaaatgagttacatttatatatatatgtatgtatgtaaacagacatagataaaagtatagatgtgtgtgtgtctttagaaaggggaggattttttttttttttttgctgtgtgttactgaagtgcctatgtctgcgtgttcacactatcatattttgtatgccctggactttataatttctaccttcaaaattagatctactgttggtaattaattcaatatatactggttttttaactactattctcatttcctagcagtaatcttcctgaaaagtcacagaaatgattacattccttgttcttcataataatcactgtttaattaaaataagaatattttagaaaagatctgcggcatagtggttaagaccccagtatttgatgctaaacagatctgatttggataacagaaggtggcactttgctgtttaagctggggaccagacactgtgggtataaatagtaattccaaacacagctccacagagcagcacccttatgacaaggttttcatatgtctatagttaagccagaaaattaagaataatgccataaatatttataaagctgaacatatccaagttaaagacctttatcctgaaattgtatcttttagattattttctaaagactaataccatttaatgtttaaatgttctttggaaatgatggtgagaatacgtgataatgggtcattggttttaatattttatttagccaagtggaaaattggcaacctggtgtcggtcctcccatttgtattttactggtgcatgaaatccaaaagtctagtaaccattgggacagacaactctactgcataagtttgtatgtttgtatatctgtatcacaaagcccagacactcgaactatataaacttgtcgcactaaagacagcaaatatgtctggtaattgcatattcttcatgtgtgcactggaatttcttattatataagaaaataaatgtgtttctaaaccaccatgaa

SEQ ID NO: 19 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 20 - protein, 1981 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 21 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 22 - protein, 1997 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 23 - protein, 1980 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 24 - protein, 1195 aa, Homo sapiens
MFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 25 - DNA, 10234 bp, Homo sapiens
  source 1..10234: mol_type = genomic DNA
gcgcgcgctccctcctctcggagagagggctgtggtaaaagccgtccggaaaatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 26 - DNA, 10343 bp, Homo sapiens
  source 1..10343: mol_type = genomic DNA
gcgcgcgctccctcctctcggagagagggctgtggtaaaagccgtccggaaaatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 27 - DNA, 10455 bp, Homo sapiens
  source 1..10455: mol_type = genomic DNA
gcgcgcgctccctcctctcggagagagggctgtggtaaaagccgtccggaaaatggccgccgccgccgccgccgcgccgagcggaggaggaggaggaggcgaggaggagagactcactgagctagcctctccttgctagaagcttacggtcgaaggtctccagccatcagaagaagcacgtggagcgctgcgtccgtgttgtggttattcatccagcatgtgttgaggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 28 - DNA, 10358 bp, Homo sapiens
  source 1..10358: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 29 - DNA, 10467 bp, Homo sapiens
  source 1..10467: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 30 - DNA, 10470 bp, Homo sapiens
  source 1..10470: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagcactgagctagcctctccttgctagaagcttacggtcgaaggtctccagccatcagaagaagcacgtggagcgctgcgtccgtgttgtggttattcatccagcatgtgttgaggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 31 - DNA, 10579 bp, Homo sapiens
  source 1..10579: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagcactgagctagcctctccttgctagaagcttacggtcgaaggtctccagccatcagaagaagcacgtggagcgctgcgtccgtgttgtggttattcatccagcatgtgttgaggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 32 - DNA, 10930 bp, Homo sapiens
  source 1..10930: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcaggacccaggagggagaagcagctgccaggggaagtctcttcgtaggcggaggtcaggagtccaagaggagtgagcagagtcacagaagcctcttaaagcctcttcttcccccatcccatcaacacatctgctgcccaagaactcctttgtgattgaatttaaccagagttgatttctgttgcttgaaactcaggaccttgatgcagtgtgcagggtctgggcaaggaaattcataaaagaagagatacagatggccaatgaagataggaaaagagttctgcctgctggtaatcaaagagatgcaaacgagaacaaaaatgatgccttttcacctaccaaatttgtcaagattaaaaagaaagcaaagagccagcgtcagctgatgttcatacctgcacctgctcggtagcttgctaatgttctgcctgctccacacgccaggccagcctccacggcgcagccagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 33 - DNA, 10635 bp, Homo sapiens
  source 1..10635: mol_type = genomic DNA
gcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagctgcaagatgggattcagatctgttctcaagcctgtcgttccaggacccaggagggagaagcagctgccaggggaagtctcttcgtaggcggaggtcaggagtccaagaggagtgagcagagtcacagaagcctcttaaagcctcttcttcccccatcccatcaacacggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 34 - DNA, 10386 bp, Homo sapiens
  source 1..10386: mol_type = genomic DNA
agtaggtggggattattatccacaaaagggactagacattgtgttctgggtcccacaactcatcataaagaggtggttatagttcccatcaggagccgtgggtaggggactgtgcgtccagcagcacccgaggctcttcggcgccagaggctctaagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtccgtttgtgtcttttgttgtaaaaatcaagtgattttttcataaggttcttttactattggaaaagatgggcagcacgcagttttattttatttttgtaagttttttaatacatgtgaaagcaaagaatactcagcatgcctttctaagtgacgcgtttgcaccttttgttgggaagtactgtatcctgtgctgttagcattctcgataaatctctctgtgaaagtga

SEQ ID NO: 35 - DNA, 10658 bp, Homo sapiens
  source 1..10658: mol_type = genomic DNA
taaaaatacagactcaccagttcctgctttgatgtgacatgtgactccccagaatacaccttgcttctgtagaccagctccaacaggattccatggtagctgggatgttagggctcaggacccaggagggagaagcagctgccaggggaagtctcttcgtaggcggaggtcaggagtccaagaggagtgagcagagtcacagaagcctcttaaagcctcttcttcccccatcccatcaacacatctgctgcccaagaactcctttgtgattgaatttaaccagagttgatttctgttgcttgaaactcaggaccttgatgcagtgtgcagggtctgggcaagtctagacttgaatgcaaacagttcttattgctaacttaccactagtcaccactaatgaacaaagactatgaacaggaaattcataaaagaagagatacagatggccaatgaagataggaaaagagttctgcctgctggtaatcaaagagatgcaaacgagaacaaaaatgatgccttttcacctaccaaatttgtcaagattaaaaagaaagcaaagagccagcgtcagctgatgttcatacctgcacctgctcggtagcttgctaatgttctgcctgctccacacgccaggccagcctccacggcgcagccagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtc

SEQ ID NO: 36 - DNA, 10141 bp, Homo sapiens
  source 1..10141: mol_type = genomic DNA
gcagtagtctcacgtggcagacccggaggtcactctcctctctggatgccctgcggacactccggttagcctcagctgcaagagcctcctcacccaaggtcacgccatttccaggacagcaccctggtgactgagcgagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccatcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaatttacataaaaatgtc

SEQ ID NO: 37 - protein, 498 aa, Homo sapiens [sequence omitted]

SEQ ID NO: 38 - protein, 390 aa, Homo sapiens
MYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTE

SEQ ID NO: 39 - protein, 263 aa, Homo sapiens
MPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 40 - protein, 486 aa, Homo sapiens
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 41 - protein, 393 aa, Homo sapiens
MYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 42 - protein, 636 aa, Homo sapiens
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 43 - protein, 547 aa, Homo sapiens
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 44 - protein, 1025 aa, Homo sapiens
MHRKKVDNRIRILIENGVAERQRSLFVVVGDRGKDQVVILHHMLSKATVKARPSVLWCYKKELGFSSHRKKRMRQLQKKIKNGTLNIKQDDPFELFIAATNIRYCYYNETHKILGNTFGMCVLQDFEALTPNLLARTVETVEGGGLVVILLRTMNSLKQLYTVTMDVHSRYRTEAHQDVVGRFNERFILSLASCKKCLVIDDQLNILPISSHVATMEALPPQTPDESLGPSDLELRELKESLQDTQPVGVLVDCCKTLDQAKAVLKFIEGISEKTLRSTVALTAARGRGKSAALGLAIAGAVAFGYSNIFVTSPSPDNLHTLFEFVFKGFDALQYQEHLDYEIIQSLNPEFNKAVIRVNVFREHRQTIQYIHPADAVKLGQAELVVIDEAAAIPLPLVKSLLGPYLVFMASTINGYEGTGRSLSLKLIQQLRQQSAQSQVSTTAENKTTTTARLASARTLYEVSLQESIRYAPGDAVEKWLNDLLCLDCLNITRIVSGCPLPEACELYYVNRDTLFCYHKASEVFLQRLMALYVASHYKNSPNDLQMLSDAPAHHLFCLLPPVPPTQNALPEVLAVIQVCLEGEISRQSILNSLSRGKKASGDLIPWTVSEQFQDPDFGGLSGGRVVRIAVHPDYQGMGYGSRALQLLQMYYEGRFPCLEEKVLETPQEIHTVSSEAVSLLEEVITPRKDLPPLLLKLNERPAERLDYLGVSYGLTPRLLKFWKRAGFVPVYLRQTPNDLTGEHSCIMLKTLTDEDEADQGGWLAAFWKDFRRRFLALLSYQFSTFSPSLALNIIQNRNMGKPAQPALSREELEALFLPYDLKRLEMYSRNMVDYHLIMDMIPAISRIYFLNQLGDLALSAAQSALLLGIGLQHKSVDQLEKEIELPSGQLMGLFNRIIRKVVKLFNEVQEKAIEEQMVAAKDVVMEPTMKTLSDDLDEAAKEFQEKHKKEVGKLKSMDLSEYIIRGDDEEWNEVLNKAGPNASIISLKSDKKRKLEAKQEPKQSKKLKNRETKNKKDMKLKRKK

SEQ ID NO: 45 - protein, 953 aa, Homo sapiens
MRQLQKKIKNGTLNIKQDDPFELFIAATNIRYCYYNETHKILGNTFGMCVLQDFEALTPNLLARTVETVEGGGLVVILLRTMNSLKQLYTVTMDVHSRYRTEAHQDVVGRFNERFILSLASCKKCLVIDDQLNILPISSHVATMEALPPQTPDESLGPSDLELRELKESLQDTQPVGVLVDCCKTLDQAKAVLKFIEGISEKTLRSTVALTAARGRGKSAALGLAIAGAVAFGYSNIFVTSPSPDNLHTLFEFVFKGFDALQYQEHLDYEIIQSLNPEFNKAVIRVNVFREHRQTIQYIHPADAVKLGQAELVVIDEAAAIPLPLVKSLLGPYLVFMASTINGYEGTGRSLSLKLIQQLRQQSAQSQVSTTAENKTTTTARLASARTLYEVSLQESIRYAPGDAVEKWLNDLLCLDCLNITRIVSGCPLPEACELYYVNRDTLFCYHKASEVFLQRLMALYVASHYKNSPNDLQMLSDAPAHHLFCLLPPVPPTQNALPEVLAVIQVCLEGEISRQSILNSLSRGKKASGDLIPWTVSEQFQDPDFGGLSGGRVVRIAVHPDYQGMGYGSRALQLLQMYYEGRFPCLEEKVLETPQEIHTVSSEAVSLLEEVITPRKDLPPLLLKLNERPAERLDYLGVSYGLTPRLLKFWKRAGFVPVYLRQTPNDLTGEHSCIMLKTLTDEDEADQGGWLAAFWKDFRRRFLALLSYQFSTFSPSLALNIIQNRNMGKPAQPALSREELEALFLPYDLKRLEMYSRNMVDYHLIMDMIPAISRIYFLNQLGDLALSAAQSALLLGIGLQHKSVDQLEKEIELPSGQLMGLFNRIIRKVVKLFNEVQEKAIEEQMVAAKDVVMEPTMKTLSDDLDEAAKEFQEKHKKEVGKLKSMDLSEYIIRGDDEEWNEVLNKAGPNASIISLKSDKKRKLEAKQEPKQSKKLKNRETKNKKDMKLKRKK

SEQ ID NO: 46 - protein, 1368 aa, synthetic construct [sequence omitted]

SEQ ID NO: 47 - protein, 1094 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQ

SEQ ID NO: 48 - protein, 1730 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQMNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 49 - protein, 1731 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDANNYPDKGVVEIKALPEIAMSIKKAFGEYAIMKGSLQLENPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 50 - DNA, 10647 bp, synthetic construct
  source 1..10647: mol_type = other DNA
ctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaacgcatacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatgcaaacaattaccccgacaaaggcgtggtggaaatcaaggccctgcctgagatcgccatgagcatcaagaaggcctttggggagtacgccatcatgaagggaagcctgcagctgcctccacttgaaagactgacactgggatccggcaagcccatccctaacccactgctgggcctggacagcaccctcgagaaccccagtgcccccagctacccgatggcctcgctctacgtgggggacctccaccccgacgtgaccgaggcgatgctctacgagaagttcagcccggccgggcccatcctctccatccgggtctgcagggacatgatcacccgccgctccttgggctacgcgtatgtgaacttccagcagccggcggacgcggagcgtgctttggacaccatgaattttgatgttataaagggcaagccagtacgcatcatgtggtctcagcgtgatccatcacttcgcaaaagtggagtaggcaacatattcattaaaaatctggacaaatccattgataataaagcactgtatgatacattttctgcttttggtaacatcctttcatgtaaggtggtttgtgatgaaaatggttccaagggctatggatttgtacactttgagacgcaggaagcagctgaaagagctattgaaaaaatgaatggaatgctcctaaatgatcgcaaagtatttgttggacgatttaagtctcgtaaagaacgagaagctgaacttggagctagggcaaaagaattcaccaatgtttacatcaagaattttggagaagacatggatgatgagcgccttaaggatctctttggcaagtttgggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagagttttctccatttggtacaatcactagtgcaaaggttatgatggaaggtggtcgcagcaaagggtttggttttgtatgtttctcctccccagaagaagccactaaagcagttacagaaatgaacggtagaattgtggccacaaagccattgtatgtagctttagctcagcgcaaagaagagcgccaggctcacctcactaaccagtatatgcagagaatggcaagtgtacgagctgttcccaaccctgtaatcaacccctaccagccagcacctccttcaggttacttcatggcagctatcccacagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcacatgctggagtctccagagtcactccgttctaaggttgatgaagctgtagctgtactacaagcccaccaagctaaagaggctgcccagaaagcagttaacagtgccaccggtgttccaactgtttaagcggccgctcgagcctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgtt

SEQ ID NO: 51 - protein, 9 aa, synthetic construct
LPPLERLTL

SEQ ID NO: 52 - DNA, 2963 bp, synthetic construct
  source 1..2963: mol_type = other DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcactatagaatagggccctctaggttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 53 - DNA, 2962 bp, synthetic construct
  source 1..2962: mol_type = other DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagctctagcatttaggtgacacgttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 54 - DNA, 2962 bp, synthetic construct
  source 1..2962: mol_type = other DNA
agtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgcaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccatgtcttcctgggacgaagacaagttgtggaaggtccagttttgaggggctattacaacttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttacc

SEQ ID NO: 55 - DNA, 28 bp, synthetic construct
  source 1..28: mol_type = other DNA
caccgcactatagaatagggccctctag

SEQ ID NO: 56 - DNA, 28 bp, synthetic construct
  source 1..28: mol_type = other DNA
caccggagctctagcatttaggtgacac

SEQ ID NO: 57 - DNA, 28 bp, synthetic construct
  source 1..28: mol_type = other DNA
caccgaaacacttagagtttcggagctt

SEQ ID NO: 58 - DNA, 28 bp, synthetic construct
  source 1..28: mol_type = other DNA
caccgtgccattcaagaagaactccgag

SEQ ID NO: 59 - DNA, 30 bp, synthetic construct
  source 1..30: mol_type = other DNA
caccgattacaccatagtaaggaagccagg

SEQ ID NO: 60 - DNA, 28 bp, synthetic construct
  source 1..28: mol_type = other DNA
caccgaaattcactgaaagaggccccca

SEQ ID NO: 61 - DNA, 28 bp, synthetic construct
  source 1..28: mol_type = other DNA
caccgacagaagattgtctgcacccgca

SEQ ID NO: 62 - DNA, 28 bp, synthetic construct
  source 1..28: mol_type = other DNA
caccggctaaaaatgtatatgcccaaag

SEQ ID NO: 63 - DNA, 28 bp, synthetic construct
  source 1..28: mol_type = other DNA
caccgccaaaagacaggttcagaagcca

SEQ ID NO: 64 - DNA, 33 bp, synthetic construct
  source 1..33: mol_type = other DNA
caccgcaccgaagttgaaagtttggaggtgcca

SEQ ID NO: 65 - DNA, 33 bp, synthetic construct
  source 1..33: mol_type = other DNA
caccgcaccgaccccagataattacaacagcca

SEQ ID NO: 66 - DNA, 28 bp, synthetic construct
  source 1..28: mol_type = other DNA
caccgcacacacataaagggcaggacag

SEQ ID NO: 67 - DNA, 28 bp, synthetic construct
  source 1..28: mol_type = other DNA
caccgaaacccaaattgaacggaaccaa

SEQ ID NO: 68 - DNA, 28 bp, synthetic construct
  source 1..28: mol_type = other DNA
caccgaagacaactgataagccaacagc

SEQ ID NO: 69 - DNA, 28 bp, synthetic construct
  source 1..28: mol_type = other DNA
caccgagccagtaaatgataggctacag

SEQ ID NO: 70 - DNA, 28 bp, synthetic construct
  source 1..28: mol_type = other DNA
caccggaataaaactgaaggccccagca

SEQ ID NO: 71 - DNA, 28 bp, synthetic construct
  source 1..28: mol_type = other DNA
caccgcagtagaaacttgttcaccacag

SEQ ID NO: 72 - DNA, 28 bp, synthetic construct
  source 1..28: mol_type = other DNA
caccgtacaagaatcaaaagtacagcag

SEQ ID NO: 73 - DNA, 41 bp, synthetic construct
  source 1..41: mol_type = other DNA
tcaagctgcccaaggaccgcatccacagcgagaagtccaac

SEQ ID NO: 74 - DNA, 41 bp, synthetic construct
  source 1..41: mol_type = other DNA
gttggacttctcgctgtggatgcggtccttgggcagcttga

SEQ ID NO: 75 - DNA, 23 bp, synthetic construct
  source 1..23: mol_type = other DNA
aatgggatcctggcaagcccatc

SEQ ID NO: 76 - DNA, 23 bp, synthetic construct
  source 1..23: mol_type = other DNA
ctaaagggaagcggccgcttaaa

SEQ ID NO: 77 - DNA, 24 bp, synthetic construct
  source 1..24: mol_type = other DNA
cagttcctggagctttgggagatt

SEQ ID NO: 78 - DNA, 25 bp, synthetic construct
  source 1..25: mol_type = other DNA
gtgtatttgatcaatccccagggaa

SEQ ID NO: 79 - DNA, 22 bp, synthetic construct
  source 1..22: mol_type = other DNA
caccattggcaatgagcggttc

SEQ ID NO: 80 - DNA, 22 bp, synthetic construct
  source 1..22: mol_type = other DNA
aggtctttgcggatgtccacgt

SEQ ID NO: 81 - DNA, 23 bp, synthetic construct
  source 1..23: mol_type = other DNA
gggatatcgttcgcaatcctcag

SEQ ID NO: 82 - DNA, 24 bp, synthetic construct
  source 1..24: mol_type = other DNA
tctaggcgctatagagcaaacagg

SEQ ID NO: 83 - DNA, 20 bp, synthetic construct
  source 1..20: mol_type = other DNA
cagagaccctcagcttccag

SEQ ID NO: 84 - DNA, 22 bp, synthetic construct
  source 1..22: mol_type = other DNA
aggatcacacccgccgagatta

SEQ ID NO: 85 - DNA, 23 bp, synthetic construct
  source 1..23: mol_type = other DNA
ctacagacttggtccgtggaatc

SEQ ID NO: 86 - DNA, 22 bp, synthetic construct
  source 1..22: mol_type = other DNA
tgagttccctcagccgttacct

SEQ ID NO: 87 - DNA, 22 bp, synthetic construct
  source 1..22: mol_type = other DNA
gaggtttcctctggtcctggta

SEQ ID NO: 88 - DNA, 22 bp, synthetic construct
  source 1..22: mol_type = other DNA
cgaaaacaggcactggaccact

SEQ ID NO: 89 - DNA, 22 bp, synthetic construct
  source 1..22: mol_type = other DNA
gatgacgactgtgtccgctgaa